(19) United States
(12) Patent Application Publication
(10) Pub. No.: US 2024/0352498 A1
(43) Pub. Date: Oct. 24, 2024

(54) Saponin production in yeast

(71) Applicants: The Regents of the University of California, Oakland, CA (US); Plant Bioscience Limited, Norwich (GB)

(72) Inventors: Yuzhong Liu, Berkeley, CA (US); Samantha Aiko Crowe, Berkeley, CA (US); Jay D. Keasling, Berkeley, CA (US); Xiaoyue Chen, Berkeley, CA (US); Graham Arthur Hudson, Berkeley, CA (US); Fei Gan, Berkeley, CA (US); Henrik V. Scheller, Berkeley, CA (US); James Reed, Norwich (GB); Laetitia Martin, Norwich (GB)

(73) Assignees: The Regents of the University of California, Oakland, CA (US); Plant Bioscience Limited, Norwich (GB)

(21) Appl. No.: 18/751,380

(22) Filed: Jun. 24, 2024

Related U.S. Application Data
(63) Continuation of application No. PCT/US22/82381, filed on Dec. 23, 2022.
(60) Provisional application No. 63/343,048, filed on May 17, 2022.
(60) Provisional application No. 63/293,748, filed on Dec. 24, 2021.
(60) Provisional application No. 63/293,747, filed on Dec. 24, 2021.

(51) Int. Cl. C12P 19/60; C12N 9/02; C12N 9/10; C12N 9/12; C12N 9/88; C12P 5/00

(52) U.S. Cl. CPC C12P 19/60; C12N 9/0069; C12N 9/0083; C12N 9/1077; C12N 9/1205; C12N 9/1241; C12N 9/88; C12P 5/007; C12Y 113/99001; C12Y 114/99; C12Y 207/01043; C12Y 207/07064; C12Y 402/01076

(57) ABSTRACT

The present invention relates inter alia to methods of biosynthetic production of QS-21, precursors and variants thereof, and to related aspects.

### CROSS-REFERENCES TO RELATED APPLICATIONS

This application is a continuation of PCT/US22/82381, filed Dec. 23, 2022, which claims priority to U.S. Provisional Application No. 63/293,747, filed Dec. 24, 2021, No. 63/293,748; filed Dec. 24, 2021, and No. 63/343,048; filed May 17, 2022, the disclosures of which are hereby incorporated by reference in its entirety for all purposes.

### REFERENCE TO A SEQUENCE LISTING

A Sequence Listing in XML format is incorporated by reference into the specification. The name of the XML file containing the Sequence Listing is B21-092-2WO.xml. The XML file is 496,599 bytes and was created on Jun. 14, 2024.

### TECHNICAL FIELD

The present invention relates to the biosynthetic production of QS-21, precursors and variants thereof, and non-native sugar in yeast, as well as to related aspects.

### BACKGROUND ART

QS-21 is a natural saponin extract from the bark of the Chilean ‘soapbark’ tree, Quillaja saponaria. QS-21 extract was originally identified as a fraction purified from a crude bark extract of Quillaja Saponaria Molina obtained by RP-HPLC purification (peak 21) (Kensil et al. 1991). Crude bark extracts have been reported to comprise a wide range of saponins. The QS-21 extract, or fraction, comprises several distinct saponin molecules. Two principal isomeric molecular constituents of the fraction were reported (Ragupathi et al. 2011) and are depicted in FIG. 1. Both incorporate a central triterpene core, or aglycon (quillaic acid), to which a branched trisaccharide is attached at the triterpene C3 oxygen functionality, and a linear tetrasaccharide is attached at the triterpene C28 carboxylate group. A fourth component within the saponin structure is a glycosylated C18 pseudo-dimeric acyl chain attached to the fucose residue of the linear tetrasaccharide terminated with an arabinofuranose residue via a hydrolytically labile ester linkage. The isomeric components differ in the constitution of the terminal sugar residue of the tetrasaccharide, in which the major and minor compounds incorporate either an apiose (65%) (‘QS-21-Api’) or a xylose (35%) (‘QS-21-Xyl’) carbohydrate, respectively (see R₂ in FIG. 1).

Saponins from Q. saponaria, including QS-21, have been known for many years to have potent immunostimulatory properties, capable of enhancing antibody production and specific T-cell responses. These properties have resulted in the development of Q. saponaria saponin-based adjuvants for vaccines. Of particular note, the AS01 adjuvant features a liposomal formulation including QS-21 and 3-O-desacyl-4′-monophosphoryl lipid A (3D-MPL) (Garcon, 2011; Didierlaurent, 2017) and is currently licenced in vaccines for diseases including shingles (Shingrix™) and malaria (Mosquirix™).

With more vaccines including QS-21 becoming available, the demand for its supply is expected to increase substantially over the years. Therefore, there remains a need for providing methods of production of QS-21 which do not rely upon natural resources, such as biosynthetic methods of production in yeast. Examples of advantages of such methods are as follows: (i) complex purification schemes designed to separate saponins from complex mixtures including multiple saponins (such as from a crude bark extract) are avoided; (ii) ability to produce individual saponins otherwise hard to separate when present in a crude bark extract (e.g. QS-21-Api and QS-21-Xyl); and (iii) ability to produce any saponin of interest (including precursors otherwise not purifiable from crude bark extracts.

The biosynthetic production of QS-21 precursors has been reported in Nicotiana benthamiana (e.g. WO 19/122259, WO 20/260475 and WO 22/136563). Quillaic acid production at trace levels has been reported in yeast (WO 20/263524). The present invention reports for the first time the successful production, in yeast, of QS-21 and glycosylated precursors and variants thereof.

### SUMMARY OF THE INVENTION

In a first aspect of the invention, there is provided a method of producing quillaic acid (QA) in yeast, wherein the method comprises the step of overexpressing, in a yeast engineered to produce β-amyrin, heterologous genes encoding the following enzymes:
a cytochrome P450 C16 oxidase, wherein the C16 oxidase oxidizes the C16 carbon of β-amyrin to a hydroxyl group,
a cytochrome P450 C23 oxidase, wherein the C23 oxidase oxidizes the C23 carbon of β-amyrin to an aldehyde group,
a cytochrome P450 C28 oxidase, wherein the C28 oxidase oxidizes the C28 carbon of β-amyrin to a carboxyl group, and
a cytochrome P450 reductase (CPR), acting as a redox partner,
wherein the C16 oxidase, the C23 oxidase, the C28 oxidase and the CPR are from a plant origin; and a yeast which is engineered to produce QA accordingly.

In a second aspect, there is provided a method of producing UDP-Glucuronic acid (UDP-GlcA) in yeast, wherein the method comprises the step of overexpressing a heterologous gene encoding a UDP-glucose dehydrogenase (UGD) converting UDP-Glucose (UDP-Glc) into UDP-GlcA; and a yeast which is engineered to produce UDP-GlcA accordingly.

In a third aspect, there is provided a method of producing UDP-Rhamnose (UDP-Rha) in yeast, wherein the method comprises the step of overexpressing a heterologous gene encoding a UDP-rhamnose synthase (RhaT) converting UDP-Glc into UDP-Rha; and a yeast which is engineered to produce UDP-Rha accordingly.

In a fourth aspect, there is provided a method of producing UDP-Xylose (UDP-Xyl) in yeast, wherein the method comprises the step of overexpressing heterologous genes encoding the following enzymes:
a UDP-glucose dehydrogenase (UGD) converting UDP-Glc into UDP-GlcA, and
a UDP-xylose synthase (UXS) converting UDP-GlcA into UDP-Xylose; and a yeast which is engineered to produce UDP-Xyl accordingly.

In a fifth aspect, there is provided a method of producing a C3-glycosylated QA derivative in yeast, wherein the method comprises the step of overexpressing, in a yeast engineered to produce QA and UDP-GlcA, a heterologous gene encoding the following enzyme:
a UDP-GlcA transferase (GlcAT) transferring UDP-GlcA and attaching a GlcA residue at the C3 position of QA to form the C3-glycosylated QA derivative;
and a yeast which is engineered to produce the C3-glycosylated QA derivative accordingly.

In a sixth aspect, there is provided a method of producing UDP-Fuc (UDP-Fuc) in yeast, wherein the method comprises the step of overexpressing heterologous genes encoding the following enzymes:
a UDP-glucose-4,6-dehydratase (UG46DH) converting UDP-Glc into UDP-4-keto-6-deoxy-glucose and
a 4-keto-reductase converting UDP-4-keto-6-deoxy-glucose into UDP-Fuc; and a yeast which is engineered to produce UDP-Fuc accordingly

In a seventh aspect, there is provided a method of producing a C28-glycosylated QA derivative in yeast, wherein the method comprises the step of overexpressing, in a yeast engineered to produce a C3-glycosylated QA derivative, a heterologous gene encoding the following enzyme:
a UDP-Fucose transferase (FucT) transferring UDP-Fuc and attaching a Fuc residue at the C28 position of the C3-glycosylated QA derivative to form the C28-glycosylated QA derivative; and a yeast which is engineered to produce the C28-glycosylated QA derivative accordingly.

In an eighth aspect, there is provided a method of producing (S)-2-methylbutyryl CoA (2 MB-CoA) in yeast, wherein the method comprises the step of overexpressing a heterologous gene encoding a carboxyl coenzyme A (CoA) ligase (CCL) converting 2-methylbutyric acid (2 MB) acid into 2 MB-CoA, and 2 MB acid is supplemented exogenously; and a yeast which is engineered to produce 2 MB-CoA accordingly.

In a ninth aspect, there is provided a method of producing UDP-Arabinofuranose (UDP-Araf) in yeast, wherein the method comprises the step of overexpressing, in a yeast engineered to produce UDP-Xyl, heterologous genes encoding the following enzymes:
a UDP-Xyl epimerase (UXE) converting UDP-Xyl into UDP-Arabinopyranose (UDP-Arap), and
a UDP-arabinose mutases (UAM) converting UDP-Arap into UDP-Arabinofuranose (UDP-Araf); and a yeast which is engineered to produce UDP-Araf accordingly.

In a tenth aspect, there is provided a method of producing an acylated and glycosylated QA derivative in yeast, wherein the method comprises the step of overexpressing, in a yeast engineered to produce a glycosylated QA derivative, heterologous genes encoding the following enzymes:
a carboxyl coenzyme A (CoA) ligase (CCL) converting 2 MB acid into 2 MB-CoA,
a chalcone-synthase-like type III polyketide synthase (PKS) condensing malonyl-CoA with 2 MB-CoA to form C9-Keto-CoA,
a keto-reductase (KR) converting C9-Keto-CoA into C9-CoA, and
an acyltransferase transferring and attaching a first C9-CoA unit to the glycosylated QA derivative to form an acylated and glycosylated QA derivative, and 2 MB acid is optionally, supplemented exogenously; and a yeast which is engineered to produce the acylated and glycosylated QA derivative accordingly.

In an eleventh aspect, there are provided QA derivatives obtained according to the method of the first to tenth aspects of the invention.

In a twelfth aspect, there is provided the use of QA derivatives according to the eleventh aspect of the invention as an adjuvant

In a thirteenth aspect, there are provided isolated enzymes or proteins used in the method of the first to tenth aspects of the invention.

### BRIEF DESCRIPTION OF THE FIGURES

FIG. 1 Shows the structure of the two principal isomeric constituents present within the QS-21 fraction traditionally purified from a crude bark extract originating from Q. saponaria Molina tree. The core backbone is formed from the triterpene quillaic acid (QA). The C3 position of QA features a branched trisaccharide consisting of a β-D-glucuronic acid (β-D-GlcA) residue, a β-D-galactose (β-D-Gal) residue and β-D-xylose (β-D-Xyl) residue at R1. The C28 position of QA features a linear tetrasaccharide consisting of a β-D-fucose (β-D-Fuc) residue, an α-L-rhamnose (α-L-Rha) residue, a β-D-xylose residue and either a terminal β-D-apiose (β-D-Api) residue or a β-D-xylose residue at R₂. The β-D-fucose residue also features an 18-carbon pseudo-dimeric acyl chain which terminates with an α-L-arabinofuranose (α-L-Ara) residue. Carbon numbering in QA (C3, C16, C23 and C28) is indicated. Substitution of R, with an α-L-rhamnose (α-L-Rha) residue represents the rhamnose-chemotype variant of QS-21, present at trace level within the QS-21 fraction traditionally purified from a crude bark extract originating from Q. saponaria Molina tree.

FIG. 2A-D Show the biosynthetic pathway for de novo production of QS-21 in yeast. FIG. 2A depicts the biosynthesis of nucleotide sugars required for the C3 branched trisaccharide and the C28 linear tetrasaccharide and the biosynthesis of the unit C9-CoA constitutive of the 18-carbon pseudo-dimeric acyl chain from the mevalonate pathway. ‘UGE’ is for UDP-glucose 4-epimerase, ‘UGD’ is for UDP-glucose dehydrogenase, ‘RHM’ is for rhamnose synthase, ‘UXS’ is for UDP-xylose synthase, ‘AXS’ is for UDP-apiose/UDP-xylose synthase, ‘UXE’ is for UDP-xyl epimerase, ‘UAM’ is for UDP-arabinose mutase. ‘LovF-TE’ is for a polyketide synthase (PKS) (or ‘megasynthase’). ‘ACP’ is for Acyl Carrier Protein. ‘TE’ is for thioesterase. ‘CCL’ is for carboxyl coenzyme A ligase, ‘PKS’ is for polyketide synthase, ‘KR’ is for keto-reductase. FIG. 2B depicts the biosynthesis of quillaic acid by successive oxidation of β-amyrin. ‘BAS’ is for β-amyrin synthase. FIG. 2C depicts the biosynthesis of the branched trisaccharide at the C3 position of QA. ‘GlcAT’ is for UDP-glucuronic acid transferase. ‘GalT’ is for UDP-galactose transferase. ‘XylT’ is for UDP-xylose transferase. ‘RhaT’ is for UDP-rhamnose transferase. FIG. 2C depicts the biosynthesis of the linear tetrasaccharide at the C28 position of QA. ‘FucT’ is for UDP-fucose transferase. FIG. 2D depicts the addition of the 18-carbon pseudo-dimeric acyl chain to the fucose residue of the linear tetrasaccaride at the C28 position of QA and the addition of arabinofuranose to the end of the acyl chain.

FIG. 3 Screening of β-amyrin synthases (BAS) from different plants. β-amyrin abundance has been measured by GC-MS in yeasts engineered with genes encoding Artemisia annua (Aa) BAS (‘AaBAS’), Arabidopsis thaliana (At) BAS (‘AtBAS’), Glycyrrhiza glabra (Gg) BAS (‘GgBAS’), and Gypsophila vaccaria (Gv) BAS (‘GvBAS’), 1 day, 2 days and 3 days after induction of gene expression.

FIG. 4 Shows the production of QA precursors (gypsogenin, oleanolic acid and hederagenin) and QA in different yeast strains (as indicated) engineered with different combinations of enzymes and proteins, as described in Table 3.

FIG. 5A-C Show a comparison of the subcellular localization of the Cytochrome P450 C28 oxidase from Quillaja saponaria (QsC28) (FIG. 5A), the Cytochrome P450 C16 oxidase from Quillaja saponaria (QsC28) (FIG. 5B), and the oxidase resulting from the fusion of QsC28 at the N-terminus of QsC16 (QSC28C16) (FIG. 5C), each tagged with a fluorescent protein at their C-terminus (GFP or mcherry, as indicated).

FIG. 6A-C A shows the relative expression level of β-amyrin synthase (SvBAS) mRNA treated by MeJa at 0, 50, 100 μM during 72 h in leaves. B shows the fold-change of β-amyrin synthase treated by MeJa at 50, 100 μM (compared to 0 μM) at 24 h and 72 h in flowers. C shows a neighbor-joining tree of cytochromes P450 (CYPs) acting on triterpenoid from other plants and CYP candidates identified from S. vaccaria transcriptome. Gene names labelled with an asterisk represent S. vaccaria genes. Gene names included in a box represent CYPs that are co-expressed with R-amyrin synthase.

FIG. 7A-C Show LC-MS extracted ion chromatograms (EIC) for QA precursors (‘oleanolic acid’, and ‘echinocystic acid’) detected in Nicotiana benthamiana plants transiently co-expressing a R-amyrin synthase from S. vaccaria (‘SvBAS’), a CYP C28 oxidase from S. vaccaria (‘SvC28’), a CYP C16 oxidase from S. vaccaria (‘SvC16’) in different combinations (as indicated) (FIG. 7A); LC-MS extracted ion chromatograms (EIC) for the QA precursor (Gypsogenin) detected in N. benthamiana plants transiently co-expressing a R-amyrin synthase from Q. saponaria (‘QsBAS’), a CYP C28 oxidase from Q. saponaria (‘QsC28’), a CYP C23 oxidase from S. vaccaria (‘SvC23-1’), a CYP C23 oxidase from S. vaccaria (‘SvC23-2’) in different combinations (as indicated) (FIG. 7B); and LC-MS extracted ion chromatograms for the QA precursor (Gypsogenic acid) detected in N. benthamiana plants transiently co-expressing the same combinations of enzymes as in Panel B (FIG. 7C).

FIG. 8 Shows LC-MS extracted ion chromatograms (EIC) for QA precursors (oleanolic acid—‘OA’, hederagenin/echinocystic acid—‘Hed/EA’, gypsogenin—‘Gyp’ and echinocystic acid ‘EA’) and QA detected in a yeast co-expressing a β-amyrin synthase (‘GvBAS’), a CYP C16 oxidase (‘SvC16’), a CYP C28 oxidase (‘QsC28’), a CYP reductase (‘AtATR1’) and a CYP oxidase C23 from S. vaccaria (‘Sv-C23-1’). The dashed line indicates that the peak obtained in the EIC for QA (in the ‘Yeast sample’) matches the peak obtained in the EIC for the QA standard (‘Commercial QA standard’). Numbers in brackets indicate m/z (mass-to-charge ratio) values.

FIG. 9 Shows LC-MS extracted ion chromatograms (EIC) for QA precursors (oleanolic acid—‘OA’, hederagenin/echinocystic acid—‘Hed/EA’, gypsogenin—‘Gyp’ and echinocystic acid ‘EA’) and QA detected in a yeast co-expressing a β-amyrin synthase (‘GvBAS’), a CYP C16 oxidase (‘SvC16’), a CYP C28 oxidase (‘QsC28’), a CYP reductase (‘AtATR1’) and a CYP oxidase C23 from S. vaccaria (‘Sv-C23-2’). The dotted line indicates that the peak obtained in the EIC for QA (in the ‘Yeast sample’) matches the peak obtained in the EIC for the QA standard (‘Commercial QA standard’). Numbers in brackets indicate m/z (mass-to-charge ratio) values.

FIG. 10 Shows the transcript expression profile of AtMSBP homologs in leaves and flowers of S. vaccaria (as indicated). Average expression levels of different homologs in leaves and flowers are represented by TMM (trimmed mean of M-values).

FIG. 11 Shows a comparison of the production of QA precursors (gypsogenin, oleanolic acid, hederagenin and erythrodiol) and QA in the absence (YL-4) or presence of MSBP proteins of different plant origins (as indicated), as measured by LC-MS.

FIG. 12 Shows the biosynthetic pathway for de novo production of nucleotide sugars in yeast via nucleotide sugar interconversion enzymes. Non-native sugars in yeast are circled. Heterologous enzymes required for synthesizing such non-native sugars are underlined. ‘Rham synthase’ is for rhamnose synthase, ‘UGE’ is for UDP-glucose 4-epimerase, ‘UGD’ is for UDP-glucose dehydrogenase, ‘UXS’ is for UDP-xylose synthase, ‘AXS’ is for UDP-apiose/UDP-xylose synthase, ‘UXE’ is for UDP-xyl epimerase, ‘UAM’ is for UDP-arabinose mutase, ‘UG46DH’ is for UDP-glucose-4,6-dehydratase, ‘UG46DGR’ is for UDP-4-keto-6-deoxy-glucose reductase.

FIG. 13A Shows a comparison of UDP-Glucose (UDP-Glc), UDP-Glucuronic acid (UDP-GlcA) and UDP-Xylose (UDP-Xyl) production between 2 yeast strains overexpressing AtUGD (a UDP-glucose dehydrogenase) (SC-1 and SC-4). FIG. 13B shows a comparison of UDP-Xyl production between 2 yeast strains overexpressing AtUGD (SC-4 and SC-16), together with a UDP-xylose synthase A. thaliana (AtUXS) and a UDP-apiose/UDP-xylose synthase from Q. saponaria (QsAXS), respectively, at 24 h and 48 h after gene expression was induced.

FIG. 14A-B Shows a comparison of UDP-Rhamnose (UDP-Rha), UDP-Xylose (UDP-Xyl) and UDP-Fucose (UDP-Fuc) production between 5 yeast strains (SC-17, SC-19, SC-20, SC-22 and SC-23) overexpressing different combinations of enzymes of different plant origins (as indicated). FIG. 14A provides results in a graph plotted against the yeast strains, while FIG. 14B provides results in a graph plotted against the UDP sugars.

FIG. 15 Shows the production of glucuronylated QA precursors (‘oleanolic acid-GlcA’, ‘gypsogenin-GlcA’ and ‘hederagenin-GlcA’) and glucuronylated QA (‘QA-C3-GlcA’) in a yeast engineered to produce QA, further overexpressing a UDP-glucuronic acid transferase from Q. saponaria (‘QsCslG1’), together with a UDP-glucose dehydrogenase from A. thaliana (‘AtUGD’) (YL-11), as measured by LC-MS. Left panel shows QA precursors and QA (unglycosylated). Right panel shows glucuronylated QA precursors and glucuronylated (‘QA-C3-GlcA’).

FIG. 16 Shows the production of glucuronylated QA precursors (‘oleanolic acid-GlcA’, ‘gypsogenin-GlcA’ and ‘hederagenin-GlcA’) and glucuronylated QA (‘QA-GlcA’) in a yeast engineered to produce QA, further overexpressing a UDP-glucuronic acid transferase (‘QsCslG2’), together with a UDP-glucose dehydrogenase (‘AtUGD’) (YL-12), as measured by LC-MS. Left panel shows QA precursors and QA (unglycosylated). Right panel shows the glucuronylated QA precursors and glucuronylated QA (‘QA-C3-GlcA’).

FIG. 17 Shows a comparison of the substrate specificity between QsCslG1 and QsCslG2. The data shown in FIG. 16 have been quantified and are presented as a graph, showing the production of QA precursors (‘OA’, ‘Her’ and ‘Gyp’), QA, glucuronylated QA precursors (‘GlcA-OA’, ‘GlcA-Her’, ‘GlcA-Gyp) and glucuronylated QA (Q‘A-C3-GlcA’) (as indicated) obtained from YL-11 (overexpressing QsCslG1) and YL-12 (overexpressing QsCslG2), respectively.

FIG. 18 Shows an LC-MS extracted ion chromatogram (EIC) for QA and QA-C3-GlcA detected in an in vitro enzymatic assay. QA and UDP-GlcA (both from a commercial source) have been directly added into a reaction buffer together with a microsome preparation of a yeast overexpressing a UDP-glucuronic transferase from S. vaccaria (‘SvCslG’) via plasmid expression.

FIG. 19 Shows LC-MS extracted ion chromatograms (EIC) for QA and C3-glycosylated QA derivatives (‘QA-C3-GlcA’ and ‘QA-C3-GlcA-Gal’) detected in a yeast engineered to produce QA-C3-GlcA, and further overexpressing a galactose transferase from Q. saponaria (‘QsGalT’) (YL-13). Peaks corresponding to QA and QA-C3-GlcA-Gal are labelled as such.

FIG. 20 Shows LC-MS extracted ion chromatograms (EIC) for C3-glycosylated QA derivatives (‘QA-C3-GlcA-Gal’ and ‘QA-C3-GlcA’) detected in N. benthamiana plants transiently co-expressing a UDP-glucuronic acid transferase from S. vaccaria (‘SvCslG’), together (or not) with a UDP-galactose transferase from S. vaccaria (‘SvGal’) (as indicated) and infiltrated with QA (from a commercial source). Peaks corresponding to QA-C3-Gal and QA-C3-GlcA-Gal are labelled as such.

FIG. 21 Shows LC-MS extracted ion chromatograms (EIC) for QA and C3-glycosylated QA derivatives (‘QA-C3-GlcA’, ‘QA-C3-GlcA-Gal’ and ‘QA-C3-GlcA-Gal-Rha’) detected in a yeast engineered to produce QA-C3-GlcA-Gal and further overexpressing a UDP-rhamnose synthase from A. thaliana (‘AtRHM2’) and a UDP-rhamnose transferase from Q. saponaria (‘QsC3RhaT’) (YL-14). Peaks corresponding to QA and QA-C3-GlcA-Gal-Rha are labelled as such.

FIG. 22 Shows LC-MS extracted ion chromatograms (EIC) for QA and C3-glycosylated QA derivatives (‘QA-C3-GlcA’, ‘QA-C3-GlcA-Gal’ and ‘QA-C3-GlcA-Gal-Xyl’) detected in a yeast engineered to produce QA-C3-GlcA-Gal and further overexpressing a UDP-xylose synthase from A. thaliana (‘AtUXS’) and a UDP-xylose transferase from Q. saponaria (‘QsC3XylT’) (YL-15). Peaks corresponding to QA and QA-C3-GlcA-Gal-Xyl are labelled as such.

FIG. 23 Shows a comparison of QA-C3-GlcA-Gal-Xyl production between 7 yeast strains (YL-15, YL-16, YL-17, YL-18, YL-19, YL-20 and YL-21) engineered to produce QA-GlcA-Gal, and further overexpressing, each, a different UDP-glucose dehydrogenase (‘UGD variants’—as indicated). ‘Syn’ is for Synechococcus sp, ‘Hs’ is for Homo sapiens, ‘Patl’ is for Paramoeba atlantica (Patl), ‘Bcyt’ is for Bacillus cytotoxicus, ‘Myxfulv’ is for Corallococcus macrosporus, ‘Pfu’ is for Pyrococcus furiosus.

FIG. 24 Shows a comparison of QA and QA-C3-GlcA-Gal-Xyl (‘QA-C3-GGX’) production between 2 yeast strains (YL-15 and YL-22) (as indicated). As compared with YL-15, YL-22 further overexpresses a glucuronkinase from A. thaliana and a UDP-glucuronic acid pyrophosphorylase from A. thaliana (‘AtUSP’).

FIG. 25 Shows a comparison of QA and QA-C3-GlcA-Gal-Xyl (‘QA-C3-GGX’) production in different yeast strains (YL-15 and YL-23) and different conditions (as indicated). As compared with YL-15, YL-23 further overexpresses a glucuronkinase from A. thaliana, a UDP-glucuronic acid pyrophosphorylase from A. thaliana (‘AtUSP’) and a myo-inositol oxygenase from Thermothelomyces thermophilus (Tt) (‘TtMIOX’). YL-23 was either left untreated (‘YL-23’) or supplemented externally with myo-inositol (‘MI’) and glucuronic acid (‘GlcA’) (as indicated).

FIG. 26 Shows a comparison of QA, QA-C3-GlcA-Gal (‘QA-C3-GG’) and QA-C3-GlcA-Gal-Xyl (‘QA-C3-GGX’) production analyzed by LC-MS. A UDP-xylose synthase from A. thaliana (‘AtUXS’) has been overexpressed in a yeast engineered to produce QA-C3-GGX under an inducible pTetOn promoter. The yeast culture was either left untreated (‘No inducer’) or treated with different concentrations of doxycycline (as indicated).

FIG. 27A shows a comparison of UDP-Xylose (‘UDP-Xy’l) and UDP-Fucose (‘UDP-Fuc’) production between different yeast strains (as indicated) overexpressing a UDP-glucose dehydrogenase from A. thaliana (‘AtUGD’), a UDP-xylose synthase from A. thaliana (‘AtUXS’), a UDP-glucose-4,6-dehydratase from S. vaccaria (‘SvUG46DH’), a UDP-4-keto-6-deoxy-glucose reductase from S. vaccaria (‘SvNMD’) and a UDP-4-keto-6-deoxy-glucose reductase from Q. saponaria in different combinations (as indicated). FIG. 27B shows a comparison of UDP-Xylose (‘UDP-Xy’l), UDP-Fucose (‘UDP-Fuc’) and UDP-Rhamnose (‘UDP-Rha’) production in different yeast strains (as indicated) overexpressing a UDP-rhamnose synthase from A. thaliana (‘AtRHM2’), a UDP-xylose synthase from A. thaliana (‘AtUXS’) a UDP-glucose dehydrogenase from A. thaliana (‘AtUGD’), a UDP-xylose synthase from A. thaliana (‘AtUXS’), a UDP-glucose-4,6-dehydratase from S. vaccaria (‘SvUG46DH’), a UDP-4-keto-6-deoxy-glucose reductase from S. vaccaria (‘SvNMD’) and a UDP-4-keto-6-deoxy-glucose reductase from Q. saponaria in different combinations (as indicated). ‘CP’ is for Cell Pellet.

FIG. 28 Shows LC-MS extracted ion chromatograms (EIC) for C3-glycosylated QA derivatives (‘QA-C3-GlcA’, ‘QA-C3-GlcA-Gal’ and ‘QA-C3-GlcA-Gal-Rha’) and a C28-glycosylated QA derivative (‘QA-C3-GlcA-Gal-Rha-C28-Fuc’) detected in a yeast engineered to produce QA-C3-GlcA-Gal-Rha and further overexpressing a UDP-glucose-4,6-dehydratase from S. vaccaria (‘SvUG46DH’), UDP-4-keto-6-deoxy-glucose reductase from S. vaccaria (‘SvNMD’) and a UDP-fucose transferase from saponaria Q. saponaria (‘QsFucT’) (YL-25). Peaks corresponding to QA-C3-GlcA-Gal-Rha and QA-C3-GlcA-Gal-Rha-C28-Fuc are labelled as such.

FIG. 29 Shows LC-MS extracted ion chromatograms (EIC) for C3-glycosylated QA derivatives (‘QA-C3-GlcA’, ‘QA-C3-GlcA-Gal’ and ‘QA-C3-GlcA-Gal-Xyl’) and C28-glycosylated QA derivatives (‘QA-C3-GlcA-Gal-Xyl-C28-Fuc′ and QA-C3-GlcA-Xyl-Rha-C28-Fuc’) detected in a yeast engineered to produce QA-C3-GlcA-Gal-Xyl-C28-Fuc and further overexpressing a UDP-rhamnose synthase from A. thaliana (‘AtRHM2’) and a UDP-rhamnose transferase from saponaria Q. saponaria (‘QsRhaT’) (YL-28). Peaks corresponding to QA-C3-GlcA-Gal, QA-C3-GlcA-Gal-Xyl and QA-C3-GlcA-Gal-Xyl-C28-Fuc-Rha are labelled as such.

FIG. 30 Shows LC-MS extracted ion chromatograms (EIC) for C3-glycosylated QA derivatives (‘QA-C3-GlcA’, ‘QA-C3-GlcA-Gal’ and ‘QA-C3-GlcA-Gal-Rha’) and C28-glycosylated QA derivatives (‘QA-C3-GlcA-Gal-Rha-C28-Fuc’ and ‘QA-C3-GlcA-Gal-Rha-C28-Fuc’) detected in a yeast engineered to produce QA-C3-GlcA-Gal-Rha-C28-Fuc and further overexpressing a UDP-rhamnose synthase from A. thaliana (‘AtRHM2’) and a UDP-rhamnose transferase from saponaria Q. saponaria (‘QsRhaT’) (YL-27). Peaks corresponding to QA-C3-GlcA-Gal-Rha and QA-C3-GlcA-Gal-Rha-C28-Fuc-Rha are labelled as such.

FIG. 31 Shows LC-MS extracted ion chromatograms (EIC) for C3-glycosylated QA derivatives (‘QA-C3-GlcA’, ‘QA-C3-GlcA-Gal’ and ‘QA-C3-GlcA-Gal-Xyl’) and C28-glycosylated QA derivatives (‘QA-C3-GlcA-Gal-Xyl-C28-Fuc’, ‘QA-C3-GlcA-Gal-Xyl-C28-Fuc-Rha’ and ‘QA-C3-GlcA-Gal-Xyl-C28-Fuc-Rha-Xyl’) detected in a yeast engineered to produce QA-C3-GlcA-Gal-Xyl-C28-Fuc-Rha and further overexpressing a UDP-xylose synthase from A. thaliana (‘AtUXS’) and a UDP-xylose transferase from Q. saponaria (‘QsC28XylT3’) (YL-30). Peaks corresponding to QA-C3-GlcA, QA-C3-GlcA-Gal, QA-C3-GlcA-Gal-Xyl, QA-C3-GlcA-Gal-Xyl-C28-Fuc-Rha and QA-C3-GlcA-Gal-Xyl-C28-Fuc-Rha-Xyl are labelled as such.

FIG. 32 Shows LC-MS extracted ion chromatograms (EIC) for C3-glycosylated QA derivatives (‘QA-C3-GlcA’, ‘QA-C3-GlcA-Gal’ and ‘QA-C3-GlcA-Gal-Rha’) and C28-glycosylated QA derivatives (‘QA-C3-GlcA-Gal-Rha-C28-Fuc’, ‘QA-C3-GlcA-Gal-Rha-C28-Fuc-Rha’ and ‘QA-C3-GlcA-Gal-Rha-C28-Fuc-Rha-Xyl’) detected in a yeast engineered to produce QA-C3-GlcA-Gal-Xyl-C28-Fuc-Rha and further overexpressing a UDP-rhamnose synthase from A. thaliana (‘AtRHM2’) and a UDP-rhamnose transferase from Q. saponaria (‘QsRhaT’) (YL-29). Peaks corresponding to QA-C3-GlcA-Gal, QA-C3-GlcA-Gal-Rha, QA-C3-GlcA-Gal-Rha-C28-Fuc-Rha and QA-C3-GlcA-Gal-Rha-C28-Fuc-Rha-Xyl are labelled as such.

FIG. 33 Shows LC-MS extracted ion chromatograms (EIC) for C3-glycosylated QA derivatives (‘QA-C3-G’, ‘QA-C3-GG’ and ‘QA-C3-GGX’) and C28-glycosylated QA derivatives (‘QA-C3-GGX-C28-FR’, ‘QA-C3-GGX-C28-FRX’ and ‘QA-C3-GGX-C28-FRXX’) detected in a yeast engineered to produce QA-C3-GGX-C28-FRX and further overexpressing a UDP-xylose synthase from A. thaliana (‘AtUXS’) and a UDP-xylose transferase from Q. saponaria (‘QsC28XylT4’) (YL-33). Peaks corresponding to QA-C3-GG, QA-C3-GGX, QA-C3-GGX-C28-FR, QA-C3-GGX-C28-FRX and QA-C3-GGX-C28-FRXX are labelled as such.

FIG. 34 Shows LC-MS extracted ion chromatograms (EIC) for C3-glycosylated QA derivatives (‘QA-C3-G’, ‘QA-C3-GG’ and ‘QA-C3-GGR’) and C28-glycosylated QA derivatives (‘QA-C3-GGR-C28-FR’, ‘QA-C3-GGR-C28-FRX’ and ‘QA-C3-GGR-C28-FRXX’) detected in a yeast engineered to produce QA-C3-GGR-C28-FRX and further overexpressing a UDP-rhamnose synthase from A. thaliana (‘AtRHM2’) and a UDP-rhamnose transferase from Q. saponaria (‘QsRhaT’) (YL-31). Peaks corresponding to QA-C3-GG, QA-C3-GGR, QA-C3-GGR-C28-FR, QA-C3-GGR-C28-FRX and QA-C3-GGR-C28-FRXX are labelled as such.

FIG. 35 Shows LC-MS extracted ion chromatograms (EIC) for C3-glycosylated QA derivatives (‘QA-C3-G’, ‘QA-C3-GG’ and ‘QA-C3-GGX’) and C28-glycosylated QA derivatives (‘QA-C3-GGX-C28-FR’, ‘QA-C3-GGX-C28-FRX’ and ‘QA-C3-GGX-C28-FRXA’) detected in a yeast engineered to produce QA-C3-GGX-C28-FRX and further overexpressing a UDP-apiose synthase from Q. saponaria (‘QsAXS’) and a UDP-apiose transferase from Q. saponaria (‘QsC28ApiT4’) (YL-34). Peaks corresponding to QA-C3-GG, QA-C3-GGX, QA-C3-GGX-C28-FR, QA-C3-GGX-C28-FRX and QA-C3-GGX-C28-FRXA are labelled as such.

FIG. 36 Shows LC-MS extracted ion chromatograms (EIC) for C3-glycosylated QA derivatives (‘QA-C3-G’, ‘QA-C3-GG’ and ‘QA-C3-GGR’) and C28-glycosylated QA derivatives (‘QA-C3-GGR-C28-FR’, ‘QA-C3-GGR-C28-FRX’ and ‘QA-C3-GGR-C28-FRXA’) detected in a yeast engineered to produce QA-C3-GGR-C28-FRX and further overexpressing UDP-apiose synthase from Q. saponaria (‘QsAXS’) and a UDP-apiose transferase from Q. saponaria (‘QsC28ApiT4’) (YL-32). Peaks corresponding to QA-C3-GG, QA-C3-GGR, QA-C3-GGR-C28-FR, QA-C3-GGR-C28-FRX and QA-C3-GGR-C28-FRXA are labelled as such.

FIG. 37 Shows a comparison of the subcellular localization of two xylose transferases from Quillaja saponaria (‘QsC28XylT3-GFP’ and (QsC28XylT4’) and an apiose transferase from Quillaja saponaria (‘QsC28ApiT-GFP’, each tagged with GFP at their C-terminus.

FIG. 38 Shows the level of protein expression (measured by fluorescence intensity after flow cytometry) of different variants of QsC28XylT4 (as indicated) which have been overexpressed in a yeast engineered to produce QA-C3-GGX-C28-FRX. ‘QsC28XylT4-3aa’, ‘QsC28XylT4-3aa’, ‘QsC28XylT4-6aa’, ‘QsC28XylT4-9aa’ and ‘QsC28XylT4-12aa’ designate variants of QsC28XylT4 having a deletion of 3, 6, 9 and 12 amino acids at the N-terminus, respectively. ‘QsC28XylT4-MBP’, ‘QsC28XylT4-SUMO’ and ‘QsC28XylT4-TrxA’ designate variants of QsC28XylT4 tagged at the N-terminus with the respective MBP, SUMO and TrxA solubility tag.

FIG. 39 Shows a comparison of QA-C3-GGX-C28-FRXX production between the yeasts overexpressing QsC28XylT4-3aa, QsC28XylT4-6aa, QsC28XylT4-9a’ and QsC28XylT4-12aa, QsC28XylT4-SUMO, QsC28XylT4-TrxA and QsC28XylT4-MBP (as indicated).

FIG. 40 Shows a comparison of the subcellular localization of unmodified QsC28XylT4 and a fusion variant (‘QsC28XylT3-3×GGGS-QsC28XylT4) having fused at the N-terminus QsC28XylT3, the two enzymes being separated by a linker (‘3×GGGS’).

FIG. 41 A-B Show LC-MS extracted ion chromatograms (EIC) for C28-glycosylated QA derivatives (‘QA-C3-GlcA-Gal-Xyl-C28-Fuc-Rha’ and ‘QA-C3-GlcA-Gal-Xyl-C28-Fuc-Rha-Xyl’) detected in a yeast engineered to produce QA-C3-GGR-C28-FRX and further overexpressing either QsC28XylT4 (FIG. 41 A) or the fusion QsC28XylT3-3×GGGS-QsC28XylT4 (YL-41) (FIG. 41B). Peaks corresponding to QA-C3-GlcA-Gal-Xyl-C28-Fuc-Rha and QA-C3-GlcA-Gal-Xyl-C28-Fuc-Rha-Xyl are labelled as such.

FIG. 42 Shows the level of protein expression of QsC28XylT4 overexpressed in yeast (measured by fluorescence intensity after flow cytometry) obtained in different culture conditions over a time period of 60 h. The yeast culture was either left untreated (‘Control’), or added with galactose, or glucose, in the same culture medium (‘old media’) or with fresh medium (‘fresh media’) (as indicated).

FIG. 43 Shows LC-MS extracted ion chromatograms (EIC) for S-2-methylbutyryl-CoA (‘2 MB-CoA’). Upper chromatogram was obtained from a ‘2M-CoA standard’. Middle chromatogram was obtained from a yeast (YL-QSCCL) overexpressing a CoA ligase from Q. saponaria (‘QsCCL’) and exogenously supplemented with 50 mg/L of 2 MB acid. Lower chromatogram was obtained from a yeast overexpressing a phosphopantetheinyl transferase from Aspergillus nidulans (‘AnNpgA’), a type I polyketide synthase (PKS) LovF from Aspergillus terreus (AstLovF-TE’) and QsCCL, in the absence of any 2 MB acid supplemented exogenously. Peaks corresponding to 2 MB-CO acid are labelled as such.

FIG. 44 Shows LC-MS extracted ion chromatograms (EIC) for acylated and/or glycosylated QA derivatives (‘QA-C3-GGX-C28-FRX-C9’, ‘QA-C3-GGX-C28-FRXX’, ‘QA-C3-GGX-C28-FRXX-C9’ and ‘QA-C3-GGX-C28-FRX) detected in a yeast (YL-42) engineered to produce QA-C3-GGX-C28-FRX, and further overexpressing chalcone-synthase-like type III polyketide synthases from Q. saponaria (‘ChsD’ and ‘ChSE’), keto-reductases from Q. saponaria (‘KR11’ and ‘KR23’), QsCCL and an acyl tranferase from Q. saponaria (‘QsDMOT9’) and exogenously supplemented with 50 mg/L of 2 MB acid. Peaks corresponding to QA-C3-GGX-C28-FRX-C9, QA-C3-GGX-C28-FRXX, QA-C3-GGX-C28-FRXX-C9 and QA-C3-GGX-C28-FRX are labelled as such.

FIG. 45 Shows a comparison of QA-C3-GGX-C28-FRX and QA-C3-GGX-C28-FRX-C9 production obtained from YL-42 in the presence of an increased concentration of 2 MB acid supplemented exogenously (as indicated).

FIG. 46 Shows LC-MS extracted ion chromatograms (EIC) for acylated and/or glycosylated QA derivatives (‘QA-C3-GGX-C28-FRX’, ‘QA-C3-GGX-C28-FR-C9‘ and’QA-C3-GGX-C28-FRXX-C18’) detected in a yeast (YL-43) engineered to produce QA-C3-GGX-C28-FRX-C9, and further overexpressing an acyl tranferase from Q. saponaria (‘QsDMOT4’) and exogenously supplemented with 500 mg/L of 2 MB acid. Peaks corresponding to QA-C3-GGX-C28-FRX, QA-C3-GGX-C28-FR-C9 and QA-C3-GGX-C28-FRXX-C18 are labelled as such.

FIG. 47 Shows LC-MS extracted ion chromatograms (EIC) for acylated and/or glycosylated QA derivatives (‘QA-C3-GGX-C28-FRX’, ‘QA-C3-GGX-C28-FRX-C9’, ‘QA-C3-GGX-C28-FRXX’, ‘QA-C3-GGX-C28-FRXX-C9′, QA-C3-GGX-C28-FRX-C18‘ and’QA-C3-GGX-C28-FRXX-C18’) detected in a yeast (YL-44) engineered to produce QA-C3-GGX-C28-FRXX-C9, and further overexpressing an acyl tranferase from Q. saponaria (‘QsDMOT4’) and exogenously supplemented with 500 mg/L of 2 MB acid. Peaks corresponding to QA-C3-GGX-C28-FRX, QA-C3-GGX-C28-FRX-C9, QA-C3-GGX-C28-FRXX-C9 and QA-C3-GGX-C28-FRXX-C18 are labelled as such.

FIG. 48A shows a comparison of UDP-Arabinopuranose (‘UDP-Arap’) and UDP-Arabinofuranose (‘UDP-Araf’) production between different yeast strains overexpressing an arabinokinase from A. thaliana (‘AtAraK’) and from Leptospira interrogans (Lei) (‘LeiAraK’), a UDP-sugar pyrophosphorylase from A. thaliana (‘AtUSP’) and from Leptospira interrogans (‘LeiUSP’), an arabinose transporter from Penicillium rubens Wisconsin (‘PrAraT’), and a UDP-arabinose mutase (‘AtUAM1’) in different combinations (as indicated). FIG. 48B shows a comparison of UDP-Arabinopuranose (‘UDP-Arap’) and UDP-Xylose (‘UDP-Xyl’) production between different yeast strains overexpressing a UDP-arabinose mutase from A. thaliana (‘AtUAM1’) and from H. vulgare (‘HvUAM’), 2 UDP-Xylose epimerase UXE from A. thaliana (‘AtUXE’ or ‘AtUXE2’) and a UDP-glucose 4-epimerase from A. thaliana (‘AtUGE3’) in different combinations (as indicated). ‘CP’ is for Cell Pellet.

FIG. 49 Shows LC-MS extracted ion chromatograms (EIC) for acylated and/or glycosylated QA derivatives (‘QA-C3-GGX-C28-FRX’, ‘QA-C3-GGX-C28-FRXX-C9′, ‘QA-C3-GGX-C28-FRX-C18′, ‘QA-C3-GGX-C28-FRX-C18-Araf, and ‘QS-21-Xyl’ corresponding to QA-C3-GGX-C28-FRX-C18-Araf) detected in a yeast (YL-45) engineered to produce QA-C3-GGX-C28-FRXX-C18, and further overexpressing a UDP-xylose epimerase from A. thaliana (‘AtUXE’) and a UDP-arabinose mutases from A. thaliana (‘AtUAM1’) and exogenously supplemented with 500 mg/L of 2 MB acid. Peaks corresponding to QA-C3-GGX-C28-FRX, QA-C3-GGX-C28-FRX-C9, QA-C3-GGX-C28-FRXX-C9 and QA-C3-GGX-C28-FRXX-C18 are labelled as such.

FIG. 50 Shows an LC-MS extracted ion chromatograms (EIC) for ‘QS-21-Xyl’ (corresponding to QA-C3-GGX-C28-FRXX-C18-Arat). Comparison is made with a QS-21 standard (QS-21 fraction purified from the bark of Q. saponaria Molina tree), with the two observed peaks matching. The inset

FIG. 51 Shows LC-MS extracted ion chromatograms (EIC) for acylated and/or glycosylated QA derivatives (‘QA-C3-GGX-C28-FRX’, ‘QA-C3-GGX-C28-FRXA’, ‘QA-C3-GGX-C28-FRX-C9′, ‘QA-C3-GGX-C28-FRXX-C9′, ‘QA-C3-GGX-C28-FRX-C18, ‘QA-C3-GGX-C28-FRXX-C18-Araf’ and ‘QS-21-Api’ corresponding to QA-C3-GGX-C28-FRX-C18-Araf) detected in a yeast (YL-46) engineered to produce QA-C3-GGX-C28-FRXA-C18, and further overexpressing a UDP-xylose epimerase from A. thaliana (‘AtUXE’), a UDP-arabinose mutase from A. thaliana (‘AtUAM1’) and an arabinofuranose transferase from Q. saponaria (‘QsArafT’) and exogenously supplemented with 500 mg/L of 2 MB acid. Peaks corresponding to QA-C3-GGX-C28-FRX, QA-C3-GGX-C28-FRX-C9 to QA-C3-GGX-C28-FRX-C18, QA-C3-GGX-C28-FRX-C18-Araf, and QS-21-Api are labelled as such.

FIG. 52 Shows LC-MS extracted ion chromatograms (EIC) for acylated and/or glycosylated QA derivatives (‘QA-C3-GGX-FRX’, ‘QA-C3-GGX-C28-FRX-C9′, ‘QA-C3-GGX-C28-FRX-C18′, and ‘QA-C3-GGX-C28-FRX-C18-Xyl’) detected in a yeast (YL-47) engineered to produce QA-C3-GGX-C28-FRX-C18, and further overexpressing an arabinofuranose transferase from Q. saponaria (‘QsArafT’) and exogenously supplemented with 500 mg/L of 2 MB acid. Peaks corresponding to QA-C3-GGX-C28-FRX-C9, QA-C3-GGX-C28-FRX-C18, and QA-C3-GGX-C28-FRX-C18-Xyl are labelled as such.

FIG. 53 Shows LC-MS extracted ion chromatograms (EIC) for acylated and glycosylated QA derivatives (‘QA-C3-GGX-FRX-C9′, ‘QA-C3-GGX-C28-FRX-C18′ and QA-C3-GGX-C28-FRX-C18-Xyl) detected in a yeast (YL-48) engineered to produce QA-C3-GGX-C28-FRX-C18, and further overexpressing an arabinofuranose transferase from Q. saponaria (‘QsArafT2’) and exogenously supplemented with 500 mg/L of 2 MB acid. Peaks corresponding to QA-C3-GGX-FRX-C9 and QA-C3-GGX-C28-FRX-C18 are labelled as such.

FIG. 54 Shows LC-MS extracted ion chromatograms (EIC) for acylated and glycosylated QA derivatives (‘QA-C3-GGX-FRX-C9′, ‘QA-C3-GGX-C28-FRX-C18′, ‘QA-C3-GGX-C28-FRX-C18-Araf and ‘QA-C3-GGX-C28-FRXX-C18-Araf) detected in a yeast (YL-49) engineered to produce QA-C3-GGX-C28-FRXX-C18, and further overexpressing an arabinofuranose transferase from Q. saponaria (‘QsArafT2’) and exogenously supplemented with 500 mg/L of 2 MB acid. Peaks corresponding to QA-C3-GGX-FRX-C9, QA-C3-GGX-C28-FRX-C18, QA-C3-GGX-C28-FRX-C18-Araf and QA-C3-GGX-C28-FRXX-C18-Araf are labelled as such.

FIG. 55 Shows LC-MS extracted ion chromatograms (EIC) for acylated and glycosylated QA derivatives (‘QS-21-Xyl’, ‘QA-C3-GGX-FRX-C9′, ‘QA-C3-GGX-C28-FRXX-C9′, ‘QA-C3-GGX-C28-FRX-C18’ and ‘QA-C3-GGX-C28-FRX-C18-Araf) detected in a yeast (YL-50) engineered to produce QA-C3-GGX-C28-FRXX-C18-Araf, and further overexpressing a phosphopantetheinyl transferase from Aspergillus nidulans (‘AnNpgA’) and a type I polyketide synthase (PKS) LovF from Aspergillus terreus (AstLovF-TE’), in the absence of any 2 MB acid supplemented exogenously. Peaks corresponding to QS-21-Xyl, QA-C3-GGX-FRX-C9, QA-C3-GGX-C28-FRX-C18 and QA-C3-GGX-C28-FRX-C18-Araf are labelled as such.

FIG. 56 Shows LC-MS extracted ion chromatograms (EIC) for acylated and glycosylated QA derivatives (‘QS-21-Api’ corresponding to QA-C3-GGX-C28-FRXA-C18-Araf, ‘QA-C3-GGX-FRXA’, ‘QA-C3-GGX-C28-FRX-C9′, ‘QA-C3-GGX-C28-FRXA-C9’ and ‘QA-C3-GGX-C28-FRX-C18) detected in a yeast (YL-51) engineered to produce QA-C3-GGX-C28-FRXX-C18-Araf, and further overexpressing a phosphopantetheinyl transferase from Aspergillus nidulans (‘AnNpgA’) and a type I polyketide synthase (PKS) LovF from Aspergillus terreus (AstLovF-TE’), in the absence of any 2 MB acid supplemented exogenously. Peaks corresponding to QS-21-Api, QA-C3-GGX-C28-FRX-C’ and QA-C3-GGX-C28-FRX-C18 are labelled as such.

FIG. 57 Shows LC-MS extracted ion chromatograms (EIC) for ‘QA-C3-GlcA-C28-Fuc′ detected in N. benthamiana plants transiently co-expressing a UDP-glucuronic acid transferase from S. vaccaria (‘SvCslG’), a UDP-glucose-4,6-dehydratase from S. vaccaria (‘SvUG46DH’), a UDP-4-keto-6-deoxy-glucose reductase from S. vaccaria (‘SvNMD’), a fucose transferase from Q. saponaria (‘QsFucT’), a fucose transferase from S. vaccaria (‘SvFucT’) and ‘GFP’ (used as negative control) in different combinations (as indicated) and infiltrated with QA (from a commercial source). Peaks corresponding to QA-C3-GlcA-C28-Fuc is labelled as such.

FIG. 58 Shows an LC-MS extracted ion chromatogram (EIC) for ‘QA-C3-GlcA-Gal-Xyl’ detected in N. benthamiana plants transiently co-expressing a UDP-glucuronic acid transferase from S. vaccaria (‘SvCslG’), a galactose transferase from S. vaccaria (‘SvGalT’), and a xylose transferase from S. vaccaria (‘SvC3XylT’), and infiltrated with QA (from a commercial source). Peaks corresponding to QA-C3-GlcA-Gal-Xyl is labelled as such.

### DETAILED DESCRIPTION OF THE INVENTION

Using more than 30 heterologous proteins from different plant and microbial origins spanning across six distinctively different protein types, including in particular a terpene synthase, cytochrome P450 monooxygenases (or ‘CYP oxidases’), nucleotide sugar synthases, sugar transferases, acyltransferases, and polyketide synthases (PKSs), the inventors have been able, for the first time, to reconstitute the metabolic pathway leading to the successful biosynthesis of QS-21 in Saccharomyces cerevisiae, starting from a simple sugar, galactose.

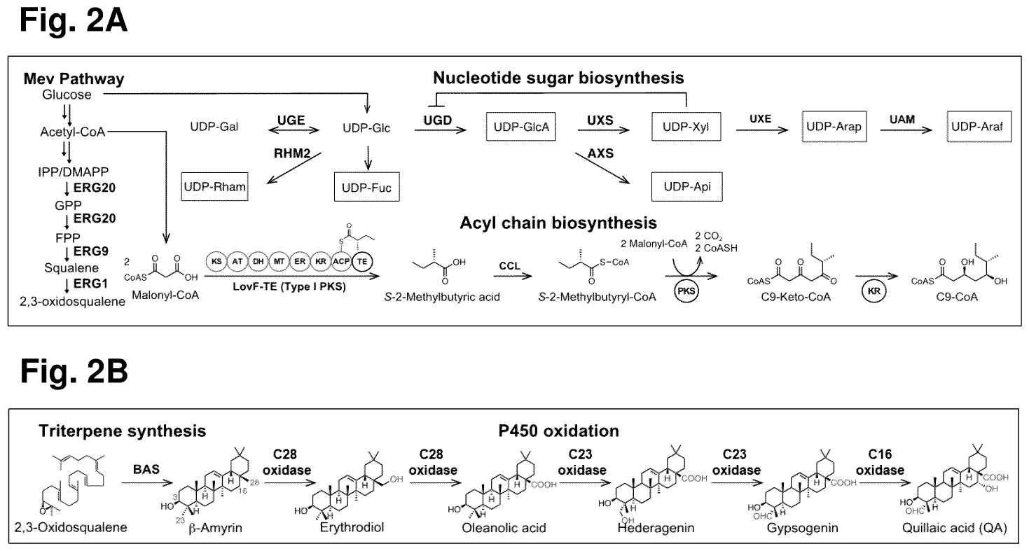

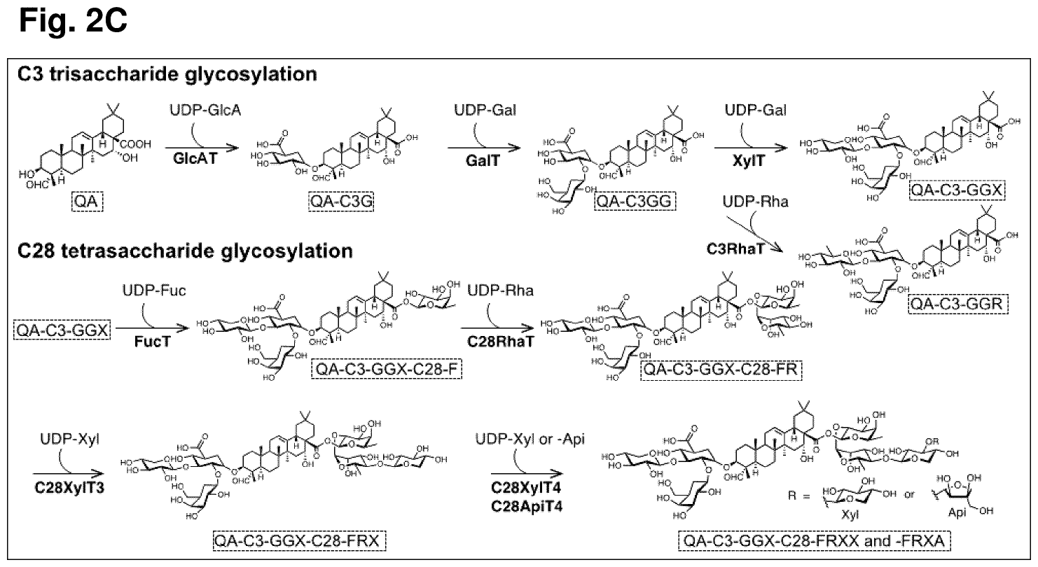

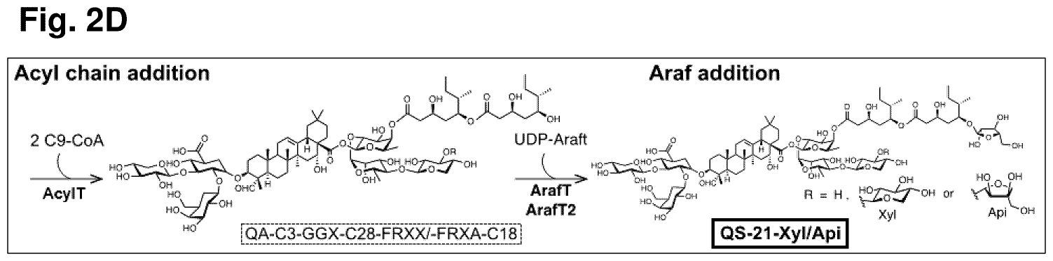

Quillaic acid (QA), the triterpene core of QS-21, derives from the simple triterpene β-amyrin, which is synthesised through cyclisation of the universal linear precursor 2,3-oxidosqualene (OS) (according to the mevalonate pathway which is native to yeast—Wong et al. 2018), by an oxidosqualene cyclase (OSC), also referred to as a β-amyrin synthase (‘BAS’) (see FIG. 2B). This β-amyrin scaffold is further oxidised with a carboxylic acid, alcohol and aldehyde at the C28, C16 and C23 positions, respectively, by a series of three CYP oxidases, resulting in the formation of quillaic acid (QA) (see FIG. 2B).

Next, UDP-Glucuronic acid (UDP-GlcA’), UDP-galactose (‘UDP-Gal’), and UDP-Xylose (‘UDP-Xyl’) or UDP-Rhamnose (‘UDP-Rha’), are incorporated at the C3 position of QA by respective glycosyltransferases resulting in the formation of C3-glycosylated QA derivatives (see FIG. 2C). Such C3-glycosylated QA derivatives are individually referred to herein as follows:
QA-C3-GlcA or QA-C3-G
QA-C3-GlcA-Gal or QA-C3-GG
QA-C3-GlcA-Gal-Rha or QA-C3-GGR
QA-C3-GlcA-Gal-Xyl or QA-C3-GGX

The formula of which being provided in Table 1.

Next, UDP-fucose (‘UDP-Fuc’), UDP-Rha, UDP-Xyl, and a second UDP-Xyl or a UDP-Api, are incorporated at the C28 position of QA by respective glycosyltransferases resulting in the formation of C28-glycosylated QA derivatives (see FIG. 2C). Such C28-glycosylated QA derivatives are individually referred to herein as follows:
QA-C3-GlcA-Gal-Xyl-C28-Fuc or QA-C3-GGX-C28-F
QA-C3-GlcA-Gal-Xyl-C28-Fuc-Rha or QA-C3-GGX-C28-FR
QA-C3-GlcA-Gal-Xyl-C28-Fuc-Rha-Xyl or QA-C3-GGX-C28-FRX
QA-C3-GlcA-Gal-Xyl-C28-Fuc-Rha-Xyl-Xyl or QA-C3-GGX-C28-FRXX
QA-C3-GlcA-Gal-Xyl-C28-Fuc-Rha-Xyl-Api or QA-C3-GGX-C28-FRXA
QA-C3-GlcA-Gal-Rha-C28-Fuc or QA-C3-GGR-C28-F
QA-C3-GlcA-Gal-Rha-C28-Fuc-Rha or QA-C3-GGR-C28-FR
QA-C3-GlcA-Gal-Rha-C28-Fuc-Rha-Xyl or QA-C3-GGR-C28-FRX
QA-C3-GlcA-Gal-Rha-C28-Fuc-Rha-Xyl-Xyl or QA-C3-GGR-C28-FRXX
QA-C3-GlcA-Gal-Rha-C28-Fuc-Rha-Xyl-Api or QA-C3-GGR-C28-FRXA

The formula of which being provided in Table 1.

Biosynthesis of the 18-carbon pseudo-dimeric acyl chain is achieved by condensing malonyl-CoA (which is native to yeast) with S-2-methylbutyryl-CoA (‘2 MB-CoA’) to make C9-CoA using a type I polyketide synthase (‘PKS’), a carboxyl coenzyme A ligase (‘CCL’), type III PKSs and keto-reductases (KRs) (see FIG. 2A).

Next, two repeating C9-CoA acyl units are successively transferred by 2 acyltransferases leading to the addition of 18-carbon pseudo-dimeric acyl chain to the fucose residue of the linear tetrasaccharide at the C28 position and resulting in the formation of acylated and glycosylated QA derivatives. Such acylated and glycosylated QA derivatives are individually referred to herein as follows:
QA-C3-GlcA-Gal-Xyl-C28-Fuc-Rha-Xyl-C9 or QA-C3-GGX-C28-FRX-C9
QA-C3-GlcA-Gal-Xyl-C28-Fuc-Rha-Xyl-Xyl-C9 or QA-C3-GGX-C28-FRXX-C9
QA-C3-GlcA-Gal-Xyl-C28-Fuc-Rha-Xyl-Api-C9 or QA-C3-GGX-C28-FRXA-C9
QA-C3-GlcA-Gal-Rha-C28-Fuc-Rha-Xyl-C9 or QA-C3-GGR-C28-FRX-C9
QA-C3-GlcA-Gal-Rha-C28-Fuc-Rha-Xyl-Xyl-C9 or QA-C3-GGR-C28-FRXX-C9
QA-C3-GlcA-Gal-Rha-C28-Fuc-Rha-Xyl-Api-C9 or QA-C3-GGR-C28-FRXA-C9
QA-C3-GlcA-Gal-Xyl-C28-Fuc-Rha-Xyl-C18 or QA-C3-GGX-C28-FRX-C18
QA-C3-GlcA-Gal-Xyl-C28-Fuc-Rha-Xyl-Xyl-C18 or QA-C3-GGX-C28-FRXX-C18
QA-C3-GlcA-Gal-Xyl-C28-Fuc-Rha-Xyl-Api-C18 or QA-C3-GGX-C28-FRXA-C18
QA-C3-GlcA-Gal-Rha-C28-Fuc-Rha-Xyl-C18 or QA-C3-GGR-C28-FRX-C18
QA-C3-GlcA-Gal-Rha-C28-Fuc-Rha-Xyl-Xyl-C18 or QA-C3-GGR-C28-FRXX-C18
QA-C3-GlcA-Gal-Rha-C28-Fuc-Rha-Xyl-Api-C18 or QA-C3-GGR-C28-FRXA-C18

The formula of which being provided in Table 1.

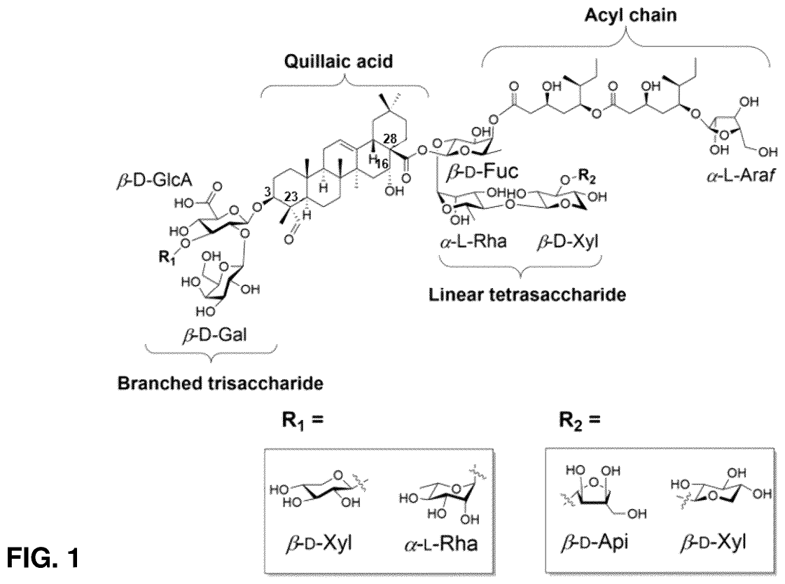

Next, UDP-arabinofuranose (‘UDP-Araf), or UDP-Xyl, is incorporated at the end of the 18-carbon pseudo-dimeric acyl chain (on the 5-hydroxy function group of the second C9-CoA acyl unit), resulting in the formation of further acylated and glycosylated QA derivatives (see FIG. 2D), including the two principal isomers of QS-21 found in the QS-21 fraction traditionally purified from the bark of the Q. saponaria Molina tree, and their rhamnose chemotype variants (see FIG. 1). Such acylated and glycosylated QA derivatives are individually referred to herein as follows:
QA-C3-GlcA-Gal-Xyl-C28-Fuc-Rha-Xyl-C18-Araf or QA-C3-GGX-C28-FRX-C18-Araf
QA-C3-GlcA-Gal-Xyl-C28-Fuc-Rha-Xyl-Xyl-C18-Araf or QA-C3-GGX-C28-FRXX-C18-Araf or QS-21-Xyl
QA-C3-GlcA-Gal-Xyl-C28-Fuc-Rha-Xyl-Api-C18-Araf or QA-C3-GGX-C28-FRXA-C18-Araf or QS-21-Api
QA-C3-GlcA-Gal-Rha-C28-Fuc-Rha-Xyl-C18-Araf or QA-C3-GGR-C28-FRX-C18-Araf
QA-C3-GlcA-Gal-Rha-C28-Fuc-Rha-Xyl-Xyl-C18-Araf or QA-C3-GGR-C28-FRXX-C18-Araf
QA-C3-GlcA-Gal-Rha-C28-Fuc-Rha-Xyl-Api-C18-Araf or QA-C3-GGR-C28-FRXA-C18-Araf
QA-C3-GlcA-Gal-Xyl-C28-Fuc-Rha-Xyl-C18-Xyl or QA-C3-GGX-C28-FRX-C18-Xyl
QA-C3-GlcA-Gal-Xyl-C28-Fuc-Rha-Xyl-Xyl-C18-Xyl or QA-C3-GGX-C28-FRXX-C18-Xyl
QA-C3-GlcA-Gal-Xyl-C28-Fuc-Rha-Xyl-Api-C18-Xyl or QA-C3-GGX-C28-FRXA-C18-Xyl
QA-C3-GlcA-Gal-Rha-C28-Fuc-Rha-Xyl-C18-Xyl or QA-C3-GGR-C28-FRX-C18-Xyl
QA-C3-GlcA-Gal-Rha-C28-Fuc-Rha-Xyl-Xyl-C18-Xyl or QA-C3-GGR-C28-FRXX-C18-Araf
QA-C3-GlcA-Gal-Rha-C28-Fuc-Rha-Xyl-Api-C18-Xyl or QA-C3-GGR-C28-FRXA-C18-Xyl

The formula of which being provided in Table 1.

‘C3-glycosylated QA derivative’ designates, in the sense of the invention, a QA derivative including at least a glucuronic acid residue at position C3 (as listed above). ‘C28-glycosylated QA derivative’ designates, in the sense of the invention, a QA derivative including all three sugars of the branched trisaccharide at position C3 and at least the fucose residue of the linear tetrasaccharide at position C28 (as listed above). ‘Acylated and glycosylated QA derivative’ designates, in the sense of the invention, a QA derivative including all three sugars of the branched trisaccharide at position C3, at least the first three sugars of the linear tetrasaccharide at position C28, at least one C9-CoA acyl unit (‘C9’) attached to the fucose residue and, optionally, an arabinofuranose residue, when two C9-CoA acyl units (‘C18’) attached (as listed above).

In the sense of the present invention, ‘heterologous genes’ is to be understood as genes not naturally expressed in yeast.

In the sense of the present invention, ‘a yeast engineered to produce e.g. a sugar or a QA derivative is to be understood as a yeast overexpressing the heterologous genes encoding the enzymes or proteins necessary to the biosynthesis or production of the respective QA derivative, e.g. as described in the respective methods of the first to tenth aspects of the invention.

### QA Production and Production Optimization

WO 19/122259 reports the identification of enzymes in the Q. saponaria genome involved in the biosynthesis of QA and the production of QA in Nicotiana benthamiana engineered with such enzymes. WO 20/263524 reports the production of traces of QA in yeast engineered with enzymes originating from different plant origins. The content of both WO 19/122259 and WO 20/263524 is incorporated herein by reference.

### β-Amyrin

The first step of the method of the first aspect of the invention is the cyclisation of 2,3-oxidosqualene to form β-amyrin. This step is carried out by an oxidosqualene cyclase or β-amyrin synthase (BAS). Any heterologous R-amyrin synthase capable of producing β-amyrin from any plant origin may suitably be used in the method of the invention. For example, β-amyrin synthases (BAS) from Artemisia annua (A. annua or ‘Aa’), Arabidopsis thaliana (A. thaliana or ‘At’), Glycyrrhiza glabra (G. glabra or ‘gG’), Gypsophila vaccaria (G. vaccaria or ‘Gv’), Medicago truncatula (M. truncatula or ‘Mt’), Quillaja saponaria (Q. saponaria or ‘Qs’), or Saponaria vaccaria (S. vaccaria or ‘Sv’) may be used. In some embodiments, the method of the first aspect of the invention uses a β-amyrin synthase selected from the foregoing plants. In particular, the β-amyrin synthase may be selected from AaBAS according to SEQ ID NO: 1, AtBAS according to SEQ ID NO: 4, GgBAS according to SEQ ID NO: 7, GvBAS according to SEQ ID NO: 10, QsBAS according to SEQ ID NO: 15 and SvBAS according to SEQ ID NO: 13. Advantageously, the β-amyrin synthase is from GvBAS according to SEQ ID NO: 10.

AaBAS, AtBAS, GgBAS, GvBAS, GvBAS, QsBAS or SvBAS may alternatively be according to sequences at least 70%, 80%, 90%, 95%, 98%, or 99% identical to SEQ ID NO: 1, SEQ ID NO: 4, SEQ ID NO: 7, SEQ ID NO: 10, SEQ ID NO: 15 or SEQ ID NO: 13.

### Quillaic Acid (QA)

As described earlier, β-amyrin is successively further oxidized with a carboxylic acid group, a hydroxyl group and aldehyde group at the C28, C16 and C23 position, respectively, by corresponding cytochrome P450 (CYP) oxidases, resulting in the formation of QA.

Any heterologous CYP oxidase from any plant origin previously identified and reported to be effectively capable of functionalizing the respective C28, C16 and C23 positions of β-amyrin may be used in the methods and engineered yeasts of the invention (e.g. as described and reported in WO 19/122259 or WO 2020/263524, or Gosh, 2017 for a review, the content of which being incorporated by reference). In some embodiments, the method of the first aspect of the invention uses a CYP C16 oxidase, a CYP C23 oxidase and a CYP C28 oxidase independently selected from A. annua, A. thaliana, G. glabra, M. truncatula, Q. saponaria, S. vaccaria, Centella asiatica, Bupleurum falcatum, Maesa lanceolate, Q. saponaria and S. vaccaria.

In further embodiments, the CYP C16 oxidase is selected from CYP87D16 and CYP716Y1; the CYP C23 oxidase is selected from CYP72A68 and CYP714E19; the CYP C28 oxidase is selected from CYP716A1, CYP716A12, CYP716A15, CYP716A17, CYP716A44, CYP716A46, CYP716A52v2, CYP716A75, CYP716A78, CYP716A79, CYP716A80, CYP716A81, CYP716A83, CYP716A86, CYP716A110, CYP716A140, CYP716A179, CYP716A252; CYP16A253 and CYP716AL1.

In further embodiments, the CYP C16 oxidase is selected from BfC16 according to SEQ ID NO: 17, QsC16 oxidase according to SEQ ID NO: 20, QsC28C16 according to SEQ ID NO: 23, and SvC16 according to SEQ ID NO: 26.

In further embodiments, the CYP C16 oxidase is selected from BfC16 according to SEQ ID NO: 17, SvC16 according to SEQ ID NO: 26, QsC16 according to SEQ ID NO: 20 and QsC28C16 according to SEQ ID NO: 23. BfC16, SvC16, QsC16 and QsC28C16 may alternatively be according to sequences at least 70%, 80%, 90%, 95%, 98%, or 99% identical to SEQ ID NO: 17, SEQ ID NO: 26, SEQ ID NO: 20 and SEQ ID NO: 23, respectively.

In further embodiments, the CYP C23 oxidase is selected from MtC23 oxidase according to SEQ ID NO: 38, QsC23 according to SEQ ID NO: 29, SvC23-1 according to SEQ ID NO: 32, and SvC23-2 according to SEQ ID NO: 35. MtC23, QsC23, SvC23-1 and SvC23-2 may alternatively be according to sequences at least 70%, 80%, 90%, 95%, 98%, or 99% identical to SEQ ID NO: 38, SEQ ID NO: 29, SEQ ID NO: 32 and SEQ ID NO: 35, respectively.

In further embodiments, the CYP C28 oxidase is selected from MtC28 according to SEQ ID NO: 46, QsC28 according to SEQ ID NO: 41, or SvC28 according to SEQ ID NO: 44. MtC28, QsC28 and SvC28 may alternatively be according to sequences at least 70%, 80%, 90%, 95%, 98%, or 99% identical to SEQ ID NO: 46, SEQ ID NO: 41 and SEQ ID NO: 44, respectively.

Heterologous redox partners, such as cytochrome P450 reductase (CPR) and/or cytochrome b5, may be further co-expressed in the method of the first aspect of the invention. For example, the CPR may be selected from A. thaliana and Lotus japonicus. In some embodiments, CPR is selected from AtATR1 according to SEQ ID NO: 49 and LjCPR according to SEQ ID NO: 52.

Heterologous cytochrome b5 may be selected from A. thaliana, Q. saponaria and S. vaccaria. In some embodiments, cytochrome b5 is selected from Atb5 according to SEQ ID NO: 58, Qsb5 according to SEQ ID NO: 55 and Svb5 according to SEQ ID NO: 61. Atb5, Qsb5 and Svb5 may alternatively be according to sequences at least 70%, 80%, 90%, 95%, 98%, or 99% identical to SEQ ID NO: 58, SEQ ID NO: 55 and SEQ ID NO: 61, respectively.

Heterologous scaffold proteins (allowing to physically organize the P450 enzymes) may be further co-expressed in the method of the first aspect of the invention. The scaffold protein may be a membrane steroid-binding protein (MSBP). For example, the MSBP may be selected from A thaliana, Q. saponaria, and S. vaccaria. In some embodiments, MSBP is selected from AtMSBP1 according to SEQ ID NO: 63, AtMSBP2 according to SEQ ID NO: 65, QsMSBP1 according to SEQ ID NO: 73, SvMSBP1 according to SEQ ID NO: 67 and SvMSBP2 according to SEQ ID NO: 70. AtMSBP2, QsMSBP1, SvMSBP1 and SvMSBP2 may alternatively be according to sequences at least 70%, 80%, 90%, 95%, 98%, or 99% identical to SEQ ID NO: 63, SEQ ID NO: 65, SEQ ID NO: 73, SEQ ID NO: 67 and SEQ ID NO: 70, respectively.

The first aspect of the invention also provides a yeast which is engineered to produce QA.

### C3 Glycosylated QA Derivatives Production

As described earlier, a branched trisaccharide consisting of GlcA, Gal and Xyl (or Rha) is attached at the C3 position of QA.

### Non-Native Sugar Production

The method according to the second aspect of the invention comprises the step of overexpressing of a heterologous gene encoding a UDP-glucose dehydrogenase (UGD) converting UDP-Glucose (UDP-Glc) into UDP-GlcA. UGD from different plant origins may be used. In some embodiments, the UGD is selected from A. thaliana, Synechococcus sp. (Syn), Homo sapiens (Hs), Paramoeba atlantica (Patl), Bacillus cytotoxicus (Bcyt), Corallococcus macrosporus (Myxfulv), and Pyrococcus furiosus (Pfu). In further embodiments, the UGD is selected from AtUGD according to SEQ ID NO: 84, AtUGD_{101L} according to SEQ ID NO: 108, SynUGD according to SEQ ID NO: 154, HsUGD_{A104L} according to SEQ ID NO: 157, PatIUGD according to SEQ ID NO: 110, BcytUGD according to SEQ ID NO: 160, MyxfulvUGD according to SEQ ID NO: 163 and PfuUGD according to SEQ ID NO: 166. AtUGD, AtUGD_{101L}, SynUGD, HsUGD_{A104L}, PatIUGD, BcytUGD, MyxfulvUGD, PfuUGD may alternatively be according to sequences at least 70%, 80%, 90%, 95%, 98%, or 99% identical to SEQ ID NO: 84, SEQ ID NO: 108, SEQ ID NO: 154, SEQ ID NO: 157, SEQ ID NO: 110, SEQ ID NO: 160, SEQ ID NO: 163 and SEQ ID NO: 166, respectively.

The second aspect of the invention also provides a yeast which is engineered to produce UDP-GlcA.

The first step of the method of the third aspect of the invention is the overexpression of a heterologous gene encoding a UDP-rhamnose synthase. A UDP-rhamnose synthase from different plant origins may be used. In some embodiments, the UDP-rhamnose synthase is AtRHM2 from A. thaliana according to SEQ ID NO: 102, or a sequence at least 70%, 80%, 90%, 95%, 98%, or 99% identical to SEQ ID NO: 102.

The third aspect of the invention also provides a yeast which is engineered to produce UDP-Rha.

The first step of the method of the fourth aspect of the invention is the overexpression of a heterologous gene encoding a UDP-glucose dehydrogenase (UGD) converting UDP-Glucose (UDP-Glc) into UDP-GlcA. The UGD may be any of the UGD described earlier in the method of the second aspect of the invention. The second step of the method of the fourth aspect of the invention is the overexpression of a heterologous gene encoding a UDP-xylose synthase (UXS). UDP-Xyl may be produced by decarboxylation of UDP-GlcA by a UDP-Xyl synthase (UXS) and/or by a dual UDP-Api/Xyl synthase (AXS). The UDP-Xylose synthase and dual UDP-Api/Xyl synthase may be from different plant origins, e.g. from A. thaliana and Q. saponaria. In some embodiments, the UXSis selected from AtUXS encoded by SEQ ID NO: 105 and QsAXS encoded by SEQ ID NO: 113, or a sequence at least 70%, 80%, 90%, 95%, 98%, or 99% identical to SEQ ID NO: 105 and SEQ ID NO: 113, respectively.

The fourth aspect of the invention also provides a yeast which is engineered to produce UDP-Xyl.

### QA-C3-GlcA Production

As shown in FIG. 2C, UDP-GlcA is transferred and a GlcA residue is attached at the C3 position of QA by a glucuronosyl transferase (GlcAT). The first step of the method of the fifth aspect of the invention is the overexpression of a heterologous gene encoding a glucuronosyl transferase (GlcAT), in a yeast engineered to produce QA and UDP-GlcA. The yeast engineered to produce QA may be a yeast according to the first aspect of the invention. The yeast engineered to produce UDP-GlcA may be a yeast according to the second aspect of the invention. The GlcAT may be from any plant origin, for example, may be selected from Q. saponaria and S. vaccaria. In some embodiments, the GlcAT is selected from OsCslG1 according to SEQ ID NO: 78, QsCslG2 according to SEQ ID NO: 81, and SvCslG according to SEQ ID NO: 76, or a sequence at least 70%, 80%, 90%, 95%, 98%, or 99% identical to SEQ ID NO: 78, SEQ ID NO: 81 and SEQ ID NO: 76, respectively.

The fifth aspect of the invention also provides a yeast which is engineered to produce QA-C3-GlcA (aspect 5a).

### QA-C3-GlcA-Gal Production

As shown in FIG. 2C, UDP-Gal is transferred and a Gal residue is attached at the C3 position of QA by a galactose transferase (GalT). The second step of the method of the fifth aspect of the invention is the overexpression of a heterologous gene encoding a galactose transferase (GalT), in a yeast engineered to produce QA-C3-GlcA. The yeast engineered to produce QA-C3-GlcA may be a yeast according to the fifth aspect of the invention. The GalT may be from any plant origin, for example, may be selected from Q. saponaria and S. vaccaria. In some embodiments, the GalT is selected from QsGalT according to SEQ ID NO: 116 and SvGalT according to SEQ ID NO: 98, or a sequence at least 70%, 80%, 90%, 95%, 98%, or 99% identical to SEQ ID NO: 116 and SEQ ID NO: 98, respectively.

The fifth aspect of the invention also provides a yeast which is engineered to produce QA-C3-GlcA-Gal (aspect 5b).

### QA-C3-GlcA-Gal-Rha Production

As shown in FIG. 2C, UDP-Rha is transferred and a Rha residue is attached at the C3 position of QA by a rhamnose transferase (RhaT). The third step of the method of the fifth aspect of the invention is the overexpression of a heterologous gene encoding a rhamnose transferase (RhaT), in a yeast engineered to produce QA-C3-GlcA-Gal and UDP-Rha. The yeast engineered to produce QA-C3-GlcA-Gal may be a yeast according to the fifth aspect of the invention. The yeast engineered to produce UDP-Rha may be a yeast according to the third aspect of the invention.

The RhaT may be from any plant origin, for example, may be from Q. saponaria. In some embodiments, the RhaT is QsRhaT according to SEQ ID NO: 119, or a sequence at least 70%, 80%, 90%, 95%, 98%, or 99% identical to SEQ ID NO: 119.

The fifth aspect of the invention also provides a yeast which is engineered to produce QA-C3-GlcA-Gal-Rha (aspect 5c).

### QA-C3-GlcA-Gal-Xyl Production

As shown in FIG. 2C, UDP-Xyl is transferred and a Xyl residue is attached at the C3 position of QA by a xylose transferase (XylT). An alternative third step of the method of the fifth aspect of the invention is the overexpression of a heterologous gene encoding a xylose transferase (XylT), in a yeast engineered to produce QA-C3-GlcA-Gal and UDP-Xyl. The yeast engineered to produce QA-C3-GlcA-Gal may be a yeast according to the aspect 5b of the invention. The yeast engineered to produce UDP-Xyl may be a yeast according to the fourth aspect of the invention. The XylT may be from any plant origin, for example, may be from Q. saponaria or S. vaccaria. In some embodiments, the XylT is selected from QsC3XylT according to SEQ ID NO: 122 and SvC3XylT according to SEQ ID NO: 100, or a sequence at least 70%, 80%, 90%, 95%, 98%, or 99% identical to SEQ ID NO: 122 or SEQ. ID NO: 100.

The fifth aspect of the invention also provides a yeast which is engineered to produce QA-C3-GlcA-Gal-Xyl (aspect 5d).

### C28-Qlycosylated QA Derivatives Production

As described earlier, a linear trisaccharide consisting of FRXX/A is attached at the C28 position of QA.

### UDP-Fuc Production

The first step of the method of the sixth aspect of the invention is the overexpression of heterologous genes encoding a UDP-glucose-4,6-dehydratase (UG46DH) converting UDP-Glc into UDP-4-keto-6-deoxy-glucose and a 4-keto-reductase converting UDP-4-keto-6-deoxy-glucose into UDP-D-Fuc. The UG46DH and 4-keto-reductase may be from any plant origin, for example, may be selected independently from Q. saponaria and S. vaccaria. In some embodiments, the UG46DH is SvUG46DH according to SEQ ID NO: 87, or a sequence at least 70%, 80%, 90%, 95%, 98%, or 99% identical to SEQ ID NO: 87. In some embodiments, the 4-keto-reductase is selected from svNMD according to SEQ ID NO: 90 and QsFucSyn according to SEQ ID NO: 175, or a sequence at least 70%, 80%, 90%, 95%, 98%, or 99% identical to SEQ ID NO: 90 or SEQ ID NO: 175.

The sixth aspect of the invention also provides a yeast which is engineered to produce UDP-Fuc.

### QA-C3-GGX-C28-F and QA-C3-GGR-C28-F Production

As shown in FIG. 2C, UDP-Fuc is transferred and a Fuc residue is attached at the C28 position of QA by a fucose transferase (FucT). The first step of the method of the seventh aspect of the invention is the overexpression of a heterologous gene encoding a fucose transferase (FucT), in a yeast engineered to produce QA-C3-GlcA-Gal-Rha, or QA-C3-GlcA-Gal-Xyl and UDP-Fucose. The yeast engineered to produce QA-C3-GlcA-Gal-Rha may be a yeast according to the aspect 5c of the invention. The yeast engineered to produce QA-C3-GlcA-Gal-Xyl may be a yeast according to the fifth aspect of the invention. The yeast engineered to produce UDP-Fuc may be a yeast according to the fifth aspect of the invention. The FucT may be selected from Q. Saponaria and S. vaccaria. In some embodiments, the FucT is selected from QsFucT according to SEQ ID NO: 93 and SvFucT according to SEQ ID NO: 96, or a sequence at least 70%, 80%, 90%, 95%, 98%, or 99% identical to SEQ ID NO: 93 and SEQ ID NO: 96, respectively.

The seventh aspect of the invention also provides a yeast which is engineered to produce QA-C3-GGR-C28-F, or QA-C3-GGX-C28-F (aspect 7a).

QA-C3-GGX-C28-FR and QA-C3-GGR-C28-FR production

As shown in FIG. 2C, UDP-Rha is transferred and a Rha residue is attached at the C28 position of QA by a Rha transferase (RhaT). The second step of the method of the seventh aspect of the invention is the overexpression of a heterologous gene encoding a rhamnose transferase (RhaT), in a yeast engineered to produce QA-C3-GGR-F, or QA-GGX-F. The yeast engineered to produce QA-C3-GGR-F or QA-C3-GGX-F may be a yeast according to the to the aspect 7a of the invention. The RhaT may be the same as described in the method of the fifth aspect of the invention.

The seventh aspect of the invention also provides a yeast which is engineered to produce QA-C3-GGR-C28-FR, or QA-C3-GGX-C28-FR (aspect 7b).

### QA-C3-GGX-C28-FRX and QA-C3-GGR-C28-FRX Production

As shown in FIG. 2C, UDP-Xyl is transferred and a Xyl residue is attached at the C28 position of QA by a xylose transferase (XylT). The third step of the method of the seventh aspect of the invention is the overexpression of a heterologous gene encoding a xylose transferase (XylT), in a yeast engineered to produce QA-C3-GGR-FR, or QA-GGX-FR. The yeast engineered to produce QA-C3-GGR-FR or QA-C3-GGX-FR may be a yeast according to the aspect 7b of the invention. The XylT may be selected from Q. saponaria and S. vaccaria. In some embodiments, XylT is QsC28XylT3 according to SEQ ID NO: 125, or a sequence at least 70%, 80%, 90%, 95%, 98%, or 99% identical to SEQ ID NO: 125.

The seventh aspect of the invention also provides a yeast which is engineered produce QA-C3-GGR-C28-FRX, or QA-C3-GGX-C28-FRX (aspect 7c).

QA-C3-GGX-C28-FRXX and QA-C3-GGR-C28-FRXX Production

As shown in FIG. 2C, UDP-Xyl is transferred and a Xyl residue is attached at the C28 position of QA by a xylose transferase (XylT). The fourth step of the method of the seventh aspect of the invention is the overexpression of a heterologous gene encoding a xylose transferase (XylT), in a yeast engineered to produce QA-C3-GGR-FRX, or QA-GGX-FRX. The yeast engineered to produce QA-C3-GGR-FRX or QA-C3-GGX-FRX may be a yeast according to the aspect 7c of the invention. XylT may be from Q. saponaria. In some embodiments, the XylT is QsC28XylT4 according to SEQ ID NO: 128, or a sequence at least 70%, 80%, 90%, 95%, 98%, or 99% identical to SEQ ID NO: 128. In further embodiments, the XylT is selected from QsC28XylT4-3aa according to SEQ ID NO: 131, QsC28XylT4-6aa according to SEQ ID NO: 134, QsC28XylT4-9aa according to SEQ ID NO: 137, and QsC28XylT4-12aa according to SEQ ID NO: 140, or a sequence at least 70%, 80%, 90%, 95%, 98%, or 99% identical to SEQ ID NO: 131, SEQ ID NO: 134, SEQ ID NO: 137 and SEQ ID NO: 140, respectively. In further embodiments, the XylT is selected from SUMO-QsC28XylT4 according to SEQ ID NO: 143, TrxA-QsC28-XylT4 according to SEQ ID NO: 145, and MBP-QsC28XylT4 according to SEQ ID NO: 147. In further embodiments, the XylT is QsC28XylT3-3×GGGS-QsC28XylT4 according to SEQ ID NO: 149.

The seventh aspect of the invention also provides a yeast which is engineered produce QA-C3-GGR-C28-FRXX, or QA-C3-GGX-C28-FRXX (aspect 7d).

### QA-C3-GGX-C28-FRXA and QA-C3-GGR-C28-FRXA Production

As shown in FIG. 2C, UDP-Api is transferred and an Api residue is attached at the C28 position of QA by an apiose transferase (XylT). An alternative fourth step of the method of the seventh aspect of the invention is the overexpression of heterologous genes encoding a UDP-apiose synthase (AXS) converting UDP-GlcA into UDP-Api and an apiose transferase (ApiT), in a yeast engineered to produce QA-C3-GGR-FRX, or QA-GGX-FRX. The yeast engineered to produce QA-C3-GGR-FRX or QA-C3-GGX-FRX may be a yeast according to the aspect 7c of the invention. The ApiT and AXS may be independently selected from Q. saponaria and S. vaccaria. In some embodiments, the AXS is QsAXS according to SEQ ID NO: 113, or a sequence at least 70%, 80%, 90%, 95%, 98%, or 99% identical to SEQ ID NO: 113. In some embodiments, the ApiT is QsC28ApiT4 according to SEQ ID NO: 151, or a sequence at least 70%, 80%, 90%, 95%, 98%, or 99% identical to SEQ ID NO: 151.

The seventh aspect of the invention also provides a yeast which is engineered to produce QA-C3-GGR-C28-FRXA, or QA-C3-GGX-C28-FRXA (aspect 7e).

Production and Attachment of the 18-Carbon Pseudo-Dimeric Acyl Chain Terminated with an Arabinofuranose (C18-Araf)

As shown in FIG. 2A, the biosynthesis of the 18-carbon pseudo-dimeric acyl chain is achieved by condensing malonyl-CoA with 2 MB-CoA to make C9-CoA.

### 2 MB-CoA Production

The method of the eighth aspect of the invention comprises the step of overexpressing a heterologous gene encoding a carboxyl coenzyme A (CoA) ligase (CCL) converting 2-methylbutyric acid (2 MB) acid into 2 MB-CoA, wherein 2 MB acid is supplemented exogenously. The CCL may be from any plant origin. In some embodiments, the CCL is QsCCL from Q. saponaria according to SEQ ID NO: 178, or a sequence at least 70%, 80%, 90%, 95%, 98%, or 99% identical to SEQ ID NO: 178. In an alternative embodiment (which does not require any exogenous supply of 2 MB acid), the method further comprises overexpressing heterologous genes encoding the following enzymes:
a phosphopantetheinyl (Ppant) transferase,
a megasynthase LovF-TE including an ACP domain, condensing two units of malonyl-CoA to 2 MB-ACP, cleaving 2 MB acid from the ACP domain which is converted into 2 MB-CoA by the CCL.

The Ppant may be from Aspergillus nidulans and the megasynthase LovF-TE may be from Aspergillus terreus. In some embodiments, the Ppant is AnNpgA according to SEQ ID NO: 237 and the megasynthase LovF-TE is AstLovF-TE according to SEQ ID NO: 235, or a sequence at least 70%, 80%, 90%, 95%, 98%, or 99% identical to SEQ ID NO: 237 and SEQ ID NO:235, respectively.

The eighth aspect of the invention also provides a yeast which is engineered to produce 2 MB-CoA.

### UDP-Arabinofuranose Production

The method according to the ninth aspect of the invention comprises the step of overexpressing, in a yeast engineered to produce UDP-Xyl, heterologous genes encoding the following enzymes:
a UDP-Xyl epimerase (UXE) converting UDP-Xyl into UDP-Arabinopyranose (UDP-Arap), and
a UDP-arabinose mutases (UAM) converting UDP-Arap into UDP-Arabinofuranose (UDP-Araf).

The yeast engineered to produce UDP-Xyl may be according to the fourth aspect of the invention.

The UXE and the UAM may be independently selected from A. thaliana and H. vulgare. In some embodiments, the UXE is selected from AtUXE according to SEQ ID NO: 199, AtUXE2 according to SEQ ID NO: 202, HvUXE-1 according to SEQ ID NO: 240, HvUXE-2 according to SEQ ID NO: 242 and AtUGE3 according to SEQ ID NO: 205, or a sequence at least 70%, 80%, 90%, 95%, 98%, or 99% identical to SEQ ID NO: 199, SEQ ID NO:202, SEQ ID NO: 240, SEQ ID NO: 242 and SEQ ID NO: 205, respectively.

In some embodiments, the UAM is selected from AtUAM1 according to SEQ ID NO: 208 and HvUAM according to SEQ ID NO: 211, or a sequence at least 70%, 80%, 90%, 95%, 98%, or 99% identical to SEQ ID NO: 208 and SEQ ID NO: 211, respectively.

The ninth aspect of the invention also provides a yeast which is engineered to produce UDP-Arabinofuranose.

### Acylated and Glycosylated QA Derivatives Production

As shown in FIG. 2A, two repeating C9-CoA acyl units are successively transferred by 2 acyltransferases leading to the addition of 18-carbon pseudo-dimeric acyl chain to the fucose residue of the linear tetrasaccharide at the C28 position and resulting in the formation of acylated and glycosylated QA derivatives.

The first step of the method of the tenth aspect of the invention is the overexpression of heterologous genes, in a yeast engineered to produce a glycosylated QA derivative, encoding the following enzymes:
a carboxyl coenzyme A ligase (CCL) converting 2 MB acid into 2 MB-CoA,
a chalcone-synthase-like type III PKS (polyketide synthase) condensing malonyl-CoA with 2 MB-CoA to form C9-Keto-CoA,
a keto-reductase (KR) converting C9-Keto-CoA into C9-CoA, and
an acyltransferase transferring and attaching a first C9-CoA unit to the glycosylated QA derivative to form an acylated and glycosylated QA derivative, and
2 MB acid is supplemented exogenously.

For example, 2 MB acid may be added directly into the yeast culture medium, at any appropriate time.

In the method according to the tenth aspect of the invention, the glycosylated QA derivative may be QA-C3-GGX-C28-FRX, QA-C3-GGR-C28-FRX, QA-C3-GGX-C28-FRXX, QA-C3-GGR-C28-FRXX, QA-C3-GGX-C28-FRXA, or QA-C3-GGR-C28-FRXA. The yeast engineered to produce QA-C3-GGX-C28-FRX and QA-C3-GGR-C28-FRX may be according to the aspect 7c. The yeast engineered to produce QA-C3-GGX-C28-FRXX and QA-C3-GGR-C28-FRXX may be according to the aspect 7d. The yeast engineered to produce QA-C3-GGX-C28-FRXA and QA-C3-GGR-C28-FRXA may be according to the aspect 7e of the invention.

In the first step of the method according to the tenth aspect of the invention, the CCL may be as described in the method of the eighth aspect of the invention.

The chalcone-synthase-like type III PKS may be form any plant origin. In some embodiments, the chalcone-synthase-like are QsChSD according to SEQ ID NO: 181, QsChSE according to SEQ ID NO: 184, or both QsChSD according to SEQ ID NO:181 and QsChSE according to SEQ ID NO: 184, or a sequence at least 70%, 80%, 90%, 95%, 98%, or 99% identical to SEQ ID NO: 181 and SEQ ID NO: 184, respectively.

The KR may be from any plant origin. In some embodiments, the KR is QsKR11 according to SEQ ID NO: 187, QsKR23 according to SEQ ID NO: 190, or both QsKR11 according to SEQ ID NO: 187 and QsKR23 according to SEQ ID NO: 190, or a sequence at least 70%, 80%, 90%, 95%, 98%, or 99% identical to SEQ ID NO: 187 and SEQ ID NO: 190, respectively.

The acyltransferase may be from any plant origin. In some embodiments, the acyltransferase is QsDMOT9 according to SEQ ID NO: 193, or a sequence at least 70%, 80%, 90%, 95%, 98%, or 99% identical to SEQ ID NO: 187 and SEQ ID NO: 193.

The tenth aspect of the invention also provides a yeast which is engineered to produce C3-GGX-C28-FRX-C9, QA-C3-GGR-C28-FRX-C9, C3-GGX-C28-FRXX-C9, QA-C3-GGR-C28-FRXX-C9, C3-GGX-C28-FRXA-C9, or QA-C3-GGR-C28-FRXA-C9 (aspect 10a).

In the second step of the method according to the tenth aspect of the invention, the acylated and glycosylated QA derivative may be C3-GGX-C28-FRX-C9, QA-C3-GGR-C28-FRX-C9, C3-GGX-C28-FRXX-C9, QA-C3-GGR-C28-FRXX-C9, C3-GGX-C28-FRXA-C9, or QA-C3-GGR-C28-FRXA-C9. The yeast engineered to produce C3-GGX-C28-FRX-C9, QA-C3-GGR-C28-FRX-C9, C3-GGX-C28-FRXX-C9, QA-C3-GGR-C28-FRXX-C9, C3-GGX-C28-FRXA-C9, or QA-C3-GGR-C28-FRXA-C9 may be according to the aspect 10a of the invention.

The third step of the method according to the tenth aspect of the invention further comprises overexpressing a gene encoding (v) a second acyltransferase attaching a second C9-CoA unit to an acylated and glycosylated QA derivative to form a further acylated and glycosylated QA derivative.

In the third step of the method according to the tenth aspect of the invention, the acylated and glycosylated QA derivative may be C3-GGX-C28-FRX-C9, QA-C3-GGR-C28-FRX-C9, C3-GGX-C28-FRXX-C9, QA-C3-GGR-C28-FRXX-C9, C3-GGX-C28-FRXA-C9, or QA-C3-GGR-C28-FRXA-C9. The yeast engineered to produce C3-GGX-C28-FRX-C9, QA-C3-GGR-C28-FRX-C9, C3-GGX-C28-FRXX-C9, QA-C3-GGR-C28-FRXX-C9, C3-GGX-C28-FRXA-C9 and QA-C3-GGR-C28-FRXA-C9 may be according to aspect 10a of the invention.

The acyltransferase may be from any plant origin. In some embodiments, the acyltransferase is QsDMOT4 according to SEQ ID NO: 196, or a sequence at least 70%, 80%, 90%, 95%, 98%, or 99% identical to SEQ ID NO: 196.

The tenth aspect of the invention also provides a yeast which is engineered to produce C3-GGX-C28-FRX-C18, QA-C3-GGR-C28-FRX-C18, C3-GGX-C28-FRXX-C18, QA-C3-GGR-C28-FRXX-C18, C3-GGX-C28-FRXA-C18, or QA-C3-GGR-C28-FRXA-C18 (aspect 10b).

The fourth step of the method according to the tenth aspect of the invention further comprises overexpressing, in a yeast engineered to produce UDP-Araf, a heterologous gene encoding (vi) an arabinotransferase (ArafT) transferring UDP-Araf and attaching an Araf residue to an acylated and glycosylated QA derivative to form an acetylated and further glycosylated QA derivative.

In the fourth step of the method according to the tenth aspect of the invention, the acylated and glycosylated QA derivative may be C3-GGX-C28-FRX-C18, QA-C3-GGR-C28-FRX-C18, C3-GGX-C28-FRXX-C18, QA-C3-GGR-C28-FRXX-C18, C3-GGX-C28-FRXA-C18, or QA-C3-GGR-C28-FRXA-C18. The yeast engineered to produce C3-GGX-C28-FRX-C18, QA-C3-GGR-C28-FRX-C18, C3-GGX-C28-FRXX-C18, QA-C3-GGR-C28-FRXX-C18, C3-GGX-C28-FRXA-C18 and QA-C3-GGR-C28-FRXA-C18 may be according to aspect 10b of the invention.

The ArafT may be from any plant origin, for example, is from Q. saponaria. In some embodiments, the ArafT is selected from QsArafT according to SEQ ID NO: 229 and QsArafT2 according to SEQ ID NO: 232, or a sequence at least 70%, 80%, 90%, 95%, 98%, or 99% identical to SEQ ID NO: 229 and SEQ ID NO: 232, respectively.

The tenth aspect of the invention also provides a yeast which is engineered to produce QA-C3-GGR-C28-FRX-C18-Araf, QA-C3-GGX-C28-FRX-C18-Araf, QA-C3-GGR-C28-FRXX-C18-Araf, QA-C3-GGX-C28-FRXX-C18-Araf, QA-C3-GGR-C28-FRXA-C18-Araf or QA-C3-GGX-C28-FRXA-C18-Araf (aspect 10c).

In embodiments, where QsArafT according to SEQ ID NO: 229 is used in the fourth step of the method according to the tenth aspect of the invention, QA-C3-GGR-C28-FRX-C18-Xyl, QA-C3-GGX-C28-FRX-C18-Xyl, QA-C3-GGR-C28-FRXX-C18-Xyl, QA-C3-GGX-C28-FRXX-C18-Xyl, QA-C3-GGR-C28-FRXA-C18-Xyl or QA-C3-GGX-C28-FRXA-C18-Xyl are also formed. The tenth aspect of the invention further provides a yeast which is engineered to produce QA-C3-GGR-C28-FRX-C18-Xyl, QA-C3-GGX-C28-FRX-C18-Xyl, QA-C3-GGR-C28-FRXX-C18-Xyl, QA-C3-GGX-C28-FRXX-C18-Xyl, QA-C3-GGR-C28-FRXA-C18-Xyl or QA-C3-GGX-C28-FRXA-C18-Xyl (aspect 10d of the invention).

In the fifth step of the method according to the tenth aspect of the invention, the method further comprises overexpressing heterologous genes encoding the following enzymes:
(vii) a phosphopantetheinyl (Ppant) transferase,
(viii) a megasynthase LovF-TE including an ACP domain, condensing two units of malonyl-CoA to 2 MB-ACP, cleaving 2 MB acid from the ACP domain which is converted into 2 MB-CoA by the CoA ligase (CCL),
and no 2 MB acid is supplemented exogenously.

The Ppant may be from Aspergillus nidulans and the megasynthase LovF-TE may be from Aspergillus terreus. In some embodiments, the Ppant is AnNpgA according to SEQ ID NO: 237 and the megasynthase LovF-TE is AstLovF-TE according to SEQ ID NO: 235, or a sequence at least 70%, 80%, 90%, 95%, 98%, or 99% identical to SEQ ID NO: 237 and SEQ ID NO: 235, respectively.

### Sequence Identity

“Percent identity” or “% identity” between a query nucleotide sequence and a subject nucleotide sequence is the “Identities” value, expressed as a percentage, that is calculated using a suitable algorithm (e.g. BLASTN, FASTA, Needleman-Wunsch, Smith-Waterman, LALIGN, or GenePAST/KERR) or software (e.g. DNASTAR Lasergene, GenomeQuest, EMBOSS needle or EMBOSS infoalign), over the entire length of the query sequence after a pair-wise global sequence alignment has been performed using a suitable algorithm (e.g. Needleman-Wunsch or GenePAST/KERR) or software (e.g. DNASTAR Lasergene or GenePAST/KERR). Importantly, a query nucleotide sequence may be described by a nucleotide sequence disclosed herein, in particular in one or more of the claims.

“Percent identity” or “% identity” between a query amino acid sequence and a subject amino acid sequence is the “Identities” value, expressed as a percentage, that is calculated using a suitable algorithm (e.g. BLASTP, FASTA, Needleman-Wunsch, Smith-Waterman, LALIGN, or GenePAST/KERR) or software (e.g. DNASTAR Lasergene, GenomeQuest, EMBOSS needle or EMBOSS infoalign), over the entire length of the query sequence after a pair-wise global sequence alignment has been performed using a suitable algorithm (e.g. Needleman-Wunsch or GenePAST/KERR) or software (e.g. DNASTAR Lasergene or GenePAST/KERR). Importantly, a query amino acid sequence may be described by an amino acid sequence disclosed herein, in particular in one or more of the claims.

The query sequence may be 100% identical to the subject sequence, or it may include up to a certain integer number of amino acid or nucleotide alterations as compared to the subject sequence such that the % identity is less than 100%. For example, the query sequence is at least 50%, 60%, 70%, 75%, 80%, 85%, 90%, 95%, 96%, 97%, 98%, or 99% identical to the subject sequence. In the case of nucleotide sequences, such alterations include at least one nucleotide residue deletion, substitution or insertion, wherein said alterations may occur at the 5′- or 3′-terminal positions of the query sequence or anywhere between those terminal positions, interspersed either individually among the nucleotide residues in the query sequence or in one or more contiguous groups within the query sequence. In the case of amino acid sequences, such alterations include at least one amino acid residue deletion, substitution (including conservative and non-conservative substitutions), or insertion, wherein said alterations may occur at the amino- or carboxy-terminal positions of the query sequence or anywhere between those terminal positions, interspersed either individually among the amino acid residues in the query sequence or in one or more contiguous groups within the query sequence.

With respect to the enzymes and/or proteins used in the methods of the invention and defined in terms of sequence identity, such enzymes and/or proteins typically retain their same respective function and activity, which function and activity may be assesses as described in the Example section.

### Yeast Engineering

Conventional methods used to engineer yeast may be used in the methods of the invention (see e.g. U.S. Pat. No. 8,828,684 B2, the content of which is incorporated by reference). Heterologous genes may be expressed under constitutive promoters or under inducible promoters, for example galactose-inducible promotors. Gene expression may be achieved either via integration into the genome of a given yeast strain (within the same locus or within different loci) or via plasmid expression. When using genome integration, one or more copies of the genes to be overexpressed may be integrated, for example, 1 to 10, 2 to 8, 3 to 7. In some embodiments, one or more of the genes involved in the biosynthesis of QS-21 are integrated into the genome of the yeast. General yeast culture conditions are known to the skilled person. Once engineered, yeast may be cultured for a few days, for example 1 to 7 days, 2 to 6 days, 4 to 5 days, or 3 days. It is within the ambit of the skilled person to determine the optimal time, depending on the metabolite to be produced. When using inducible promoters such as the gal promoters, determining the optimal induction time is also within the ambit of the skilled person. At any appropriate time after culture and/or induction, the desired metabolites, e.g. sugars or the QA derivatives of the invention may be recovered from the yeast culture, by any methods known in the art, such as extraction using a non-aqueous polar solvent, extraction using an acid medium or a basic medium, or recovery by resin absorption, or extraction by mechanically disrupting the plant cells, such as by ball milling or sonication. In some embodiments, the yeast is Saccharomyces cerevisiae.

### Adjuvants

The QA derivatives of the invention may be used as an adjuvant, individually, or in any combination. They may also be combined with further immuno-stimulants, in particular with a TLR4 agonist. In some embodiments, the QA derivatives are formulated within a liposome, in combination with a TLR4 agonist.

The TLR4 agonist may be 3D-MPL, in particular lipopolysaccharide TLR4 agonists, such as lipid A derivatives, especially a monophosphoryl lipid A, e.g. 3-de-O-acylated monophosphoryl lipid A (3D-MPL). 3D-MPL is sold under the name ‘MPL’ by GlaxoSmithKline Biologicals N.A. See, for example, U.S. Pat. Nos. 4,436,727; 4,877,611; 4,866,034 and 4,912,094. 3D-MPL can be produced according to the methods described in GB 2 220 211 A. Chemically, it is a mixture of 3-deacylated monophosphoryl lipid A with 4, 5 or 6 acylated chains.

Adjuvants of the invention may also be formulated into a suitable carrier, such as an emulsion (e.g. an oil-in-water emulsion) or liposomes, as described below.

### Liposomes

The term liposome is well known in the art and defines a general category of vesicles which comprise one or more lipid bilayers surrounding an aqueous space. Liposomes thus consist of one or more lipid and/or phospholipid bilayers and can contain other molecules, such as proteins or carbohydrates, in their structure. Because both lipid and aqueous phases are present, liposomes can encapsulate or entrap water-soluble material, lipid-soluble material, and/or amphiphilic compounds. A method for making such liposomes is described in WO 13/041572.

Liposome size may vary from 30 nm to several μm depending on the phospholipid composition and the method used for their preparation.

The liposome size will be in the range of 50 nm to 200 nm, especially 60 nm to 180 nm, such as 70-165 nm. Optimally, the liposomes should be stable and have a diameter of 100 nm to allow convenient sterilization by filtration.

Structural integrity of the liposomes may be assessed by methods such as dynamic light scattering (DLS) measuring the size (Z-average diameter, Zav) and polydispersity of the liposomes, or, by electron microscopy for analysis of the structure of the liposomes. The average particle size may be between 95 and 120 nm, and/or, the polydispersity (Pdl) index may not be more than 0.3 (such as not more than 0.2).

TABLE 1
QA and acylated and/or glycosylated QA derivatives

| | |
|---|---|
| QA | Quillaic acid |

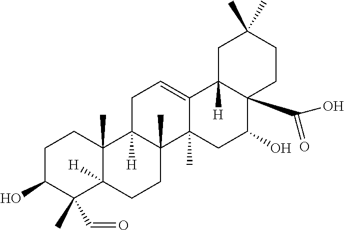

| | |
|---|---|
| QA-C3-GlcA (or QA-C3-G) | 3-O-{β-D-glucopyranosiduronic acid}-quillaic acid |

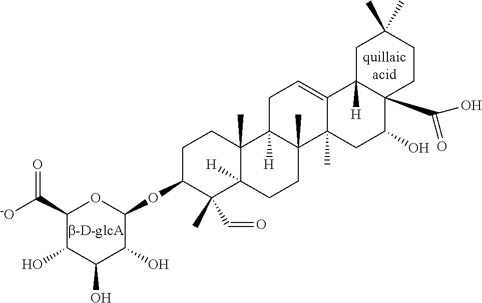

Chemical Formula: C₃₆H₅₃O₁₁⁻ Exact Mass: 661,36

| | |
|---|---|
| QA-C3-GlcA-Gal (or QA-C3-GG) | 3-O-{β-D-galactopyranosyl-(1->2)-β-D-glucopyranosiduronic |
| | acid}-quillaic acid |

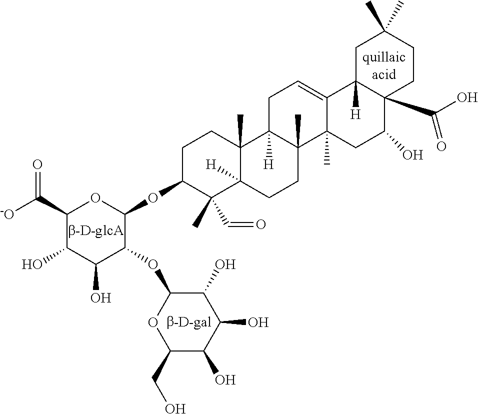

Chemical Formula: C₄₂H₆₃O₁₆⁻ Exact Mass: 823,41

| | |
|---|---|
| QA-C3-GlcA-Gal-Rha (or QA-C3- | 3-O-{α-L-rhamnopyranosyl-(1->3)-[β-D-galactopyranosyl- |
| GGR) | (1->2)]-β-D-glucopyranosiduronic acid}-quillaic acid |

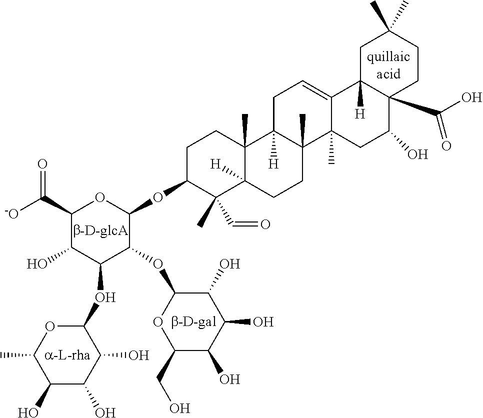

Chemical Formula: C₄₈H₇₃O₂₀⁻ Exact Mass: 969,47

| | |
|---|---|
| QA-C3-GlcA-Gal-Xyl (or QA-C3-GGX) | 3-O-{β-D-xylopyranosyl-(1->3)-[β-D-galactopyranosyl- |
| | (1->2)]-β-D-glucopyranosiduronic acid}-quillaic acid |

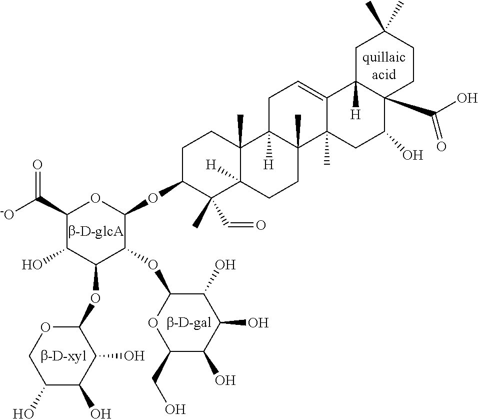

Chemical Formula: C₄₇H₇₁O₂₀⁻ Exact Mass: 955,45

| | |
|---|---|
| QA-C3-GlcA-Gal-Rha-C28-Fuc | 3-O-{α-L-rhamnopyranosyl-(1->3)-[β-D-galactopyranosyl- |
| (or QA-C3-GGR-C28-F) | (1->2)]-β-D-glucopyranosiduronic acid}-28-O-{β-D- |
| | fucopyranosyl ester}-quillaic acid |

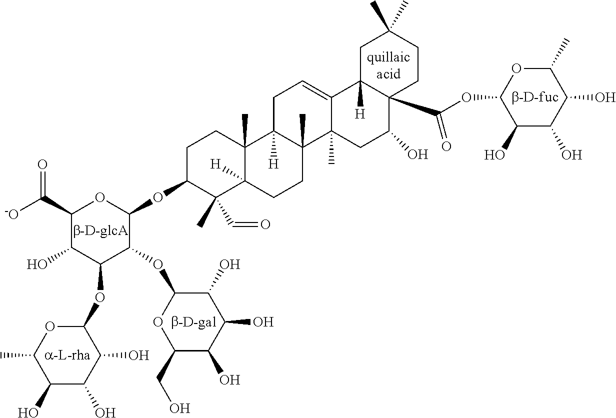

Chemical Formula: C₅₄H₈₃O₂₄⁻ Exact Mass: 1115,53

| | |
|---|---|
| QA-C3-GlcA-Gal-Xyl-C28-Fuc | 3-O-{β-D-xylopyranosyl-(1->3)-[β-D-galactopyranosyl- |
| (or QA-C3-GGX-C28-F) | (1->2)]-β-D-glucopyranosiduronic acid}-28-O-{β-D- |
| | fucopyranosyl ester}-quillaic acid |

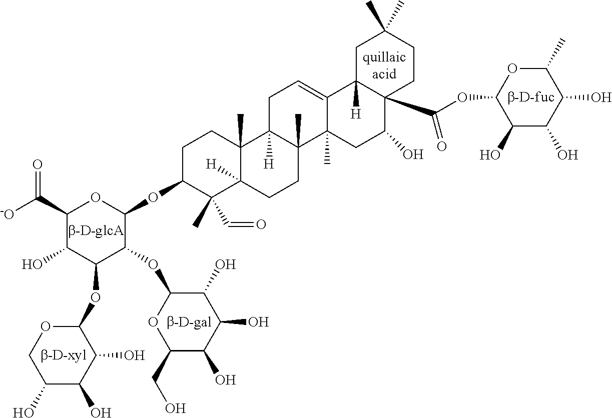

Chemical Formula: C₅₃H₈₁O₂₄⁻ Exact Mass: 1101,51

| | |
|---|---|
| QA-C3-GlcA-Gal-Rha-C28-Fuc-Rha | 3-O-{α-L-rhamnopyranosyl-(1->3)-[β-D-galactopyranosyl- |
| (or QA-C3-GGR-C28-FR) | (1->2)]-β-D-glucopyranosiduronic acid}-28-O-{α-L- |
| | rhamnopyranosyl-(1->2)-β-D-fucopyranosyl ester}-quillaic |
| | acid |

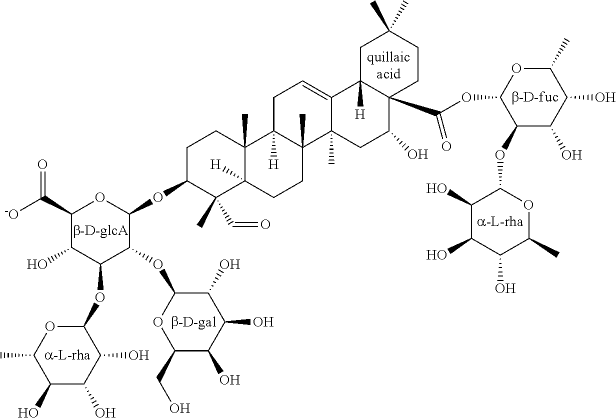

Chemical Formula: C₆₀H₉₃O₂₈⁻ Exact Mass: 1261,59

| | |
|---|---|
| QA-C3-GlcA-Gal-Xyl-C28-Fuc-Rha | 3-O-{β-D-xylopyranosyl-(1->3)-[β-D-galactopyranosyl- |
| (or QA-C3-GGX-C28-FR) | (1->2)]-β-D-glucopyranosiduronic acid}-28-O-(α-L- |
| | rhamnopyranosyl-(1->2)-β-D-fucopyranosyl ester}-quillaic |
| | acid |

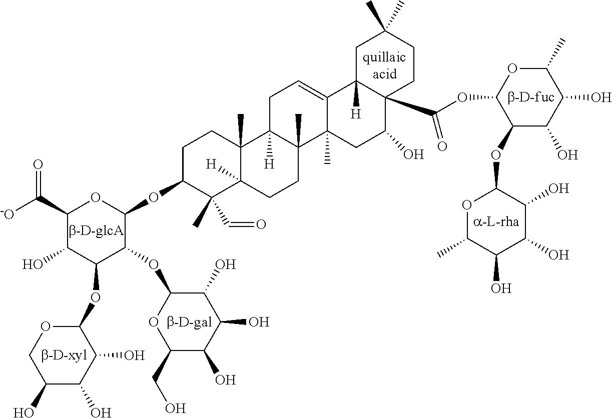

Chemical Formula: C₅₉H₉₁O₂₈⁻ Exact Mass: 1247,57

| | |
|---|---|
| QA-C3-GlcA-Gal-Rha-C28-Fuc-Rha- | 3-O-{α-L-rhamnopyranosyl-(1->3)-[β-D-galactopyranosyl- |
| Xyl | (1->2)]-[β-D-glucopyranosiduronic acid}-28-O-{β-D- |
| (or QA-C3-GGR-C28-FRX) | xylopyranosyl-(1->4)-α-L-rhamnopyranosyl-(1->2)-β-D- |
| | fucopyranosyl ester}-quillaic acid |

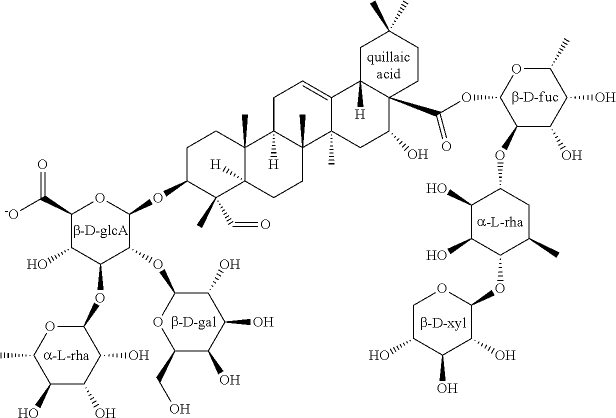

Chemical Formula: C₆₅H₁₀₁O₃₂⁻ Exact Mass: 1393,63

| | |
|---|---|
| QA-C3-GlcA-Gal-Xyl-C28-Fuc-Rha- | 3-O-{β-D-xylopyranosyl-(1->3)-[β-D-galactopyranosyl- |
| Xyl | (1->2)]-β-D-glucopyranosiduronic acid}-28-O-{β-D- |
| (or QA-C3-GGX-C28-FRX) | xylopyranosyl-(1->4)-α-L-rhamnopyranosyl-(1->2)-β-D- |
| | fucopyranosyl ester}-quillaic acid |

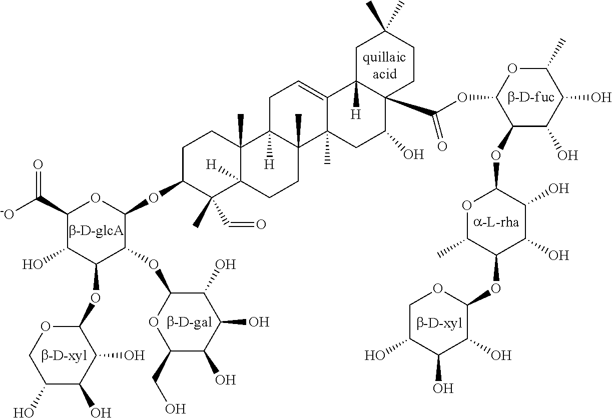

Chemical Formula: C₆₄H₉₉O₃₂⁻ Exact Mass: 1379,61

| | |
|---|---|
| QA-C3-GlcA-Gal-Rha-C28-Fuc-Rha- | 3-O-{α-L-rhamnopyranosyl-(1->3)-[β-D-galactopyranosyl- |
| Xyl-Xyl | (1->2)]-β-D-glucopyranosiduronic acid}-28-O-{β-D- |
| (or QA-C3-GGR-C28-FRXX) | xylopyranosyl-(1->3)-β-D-xylopyranosyl-(1->4)-α-L- |
| | rhamnopyranosyl-(1->2)-β-D-fucopyranosyl ester}-quillaic |
| | acid |

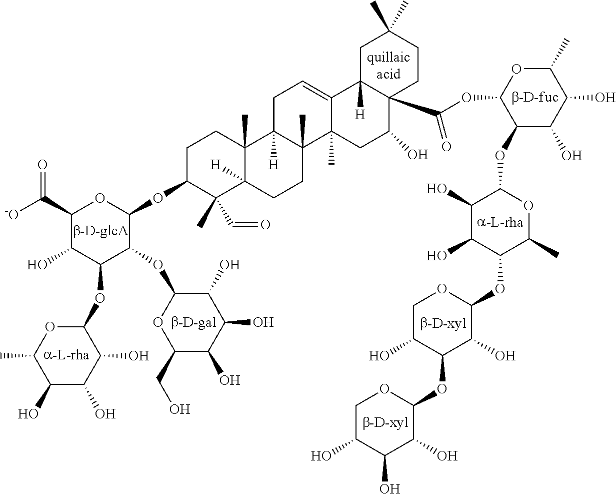

Chemical Formula: C₇₀H₁₀₉O₃₆⁻ Exact Mass: 1525,67

| | |
|---|---|
| QA-C3-GlcA-Gal-Xyl-C28-Fuc-Rha- | 3-O-{β-D-xylopyranosyl-(1->3)-[β-D-galactopyranosyl- |
| Xyl-Xyl | (1->2)]-β-D-glucopyranosiduronic acid}-28-O-{β-D- |
| (or QA-C3-GGX-C28-FRXX) | xylopyranosyl-(1->3)-β-D-xylopyranosyl-(1->4)-α-L- |
| | rhamnopyranosyl-(1->2)-β-D-fucopyranosyl ester)-quillaic |
| | acid |

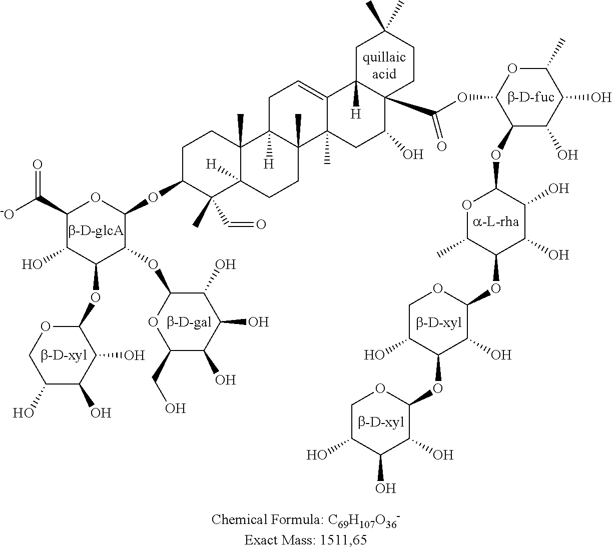

| | |
|---|---|
| QA-C3-GlcA-Gal-Rha-C28-Fuc-Rha- | 3-O-{α-L-rhamnopyranosyl-(1->3)-[β-D-galactopyranosyl- |
| Xyl-Api | (1->2)]-3-D-glucopyranosiduronic acid}-28-O-{β-D- |
| (or QA-C3-GGR-C28-FRXA) | apiofuranosyl-(1->3)-β-D-xylopyranosyl-(1->4)-α-L- |
| | rhamnopyranosyl-(1->2)-β-D-fucopyranosyl ester}-quillaic |
| | acid |

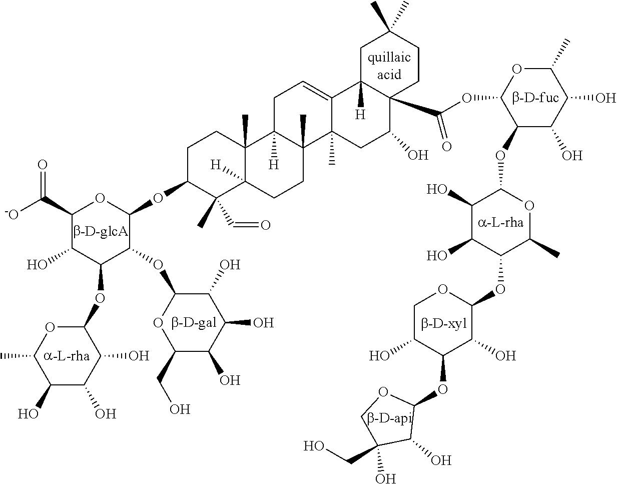

Chemical Formula: C70H109O36- Exact Mass: 1525,67

| | |
|---|---|
| QA-C3-GlcA-Gal-Xyl-C28-Fuc-Rha- | 3-O-{β-D-xylopyranosyl-(1->3)-[β-D-galactopyranosyl- |
| Xyl-Api | (1->2)]-β-D-glucopyranosiduronic acid}-28-O-{β-D- |
| (or QA-C3-GGX-C28-FRXA) | apiofuranosyl-(1->3)-β-D-xylopyranosyl-(1->4)-α-L- |
| | rhamnopyranosyl-(1->2)-β-D-fucopyranosyl ester)-quillaic |
| | acid |

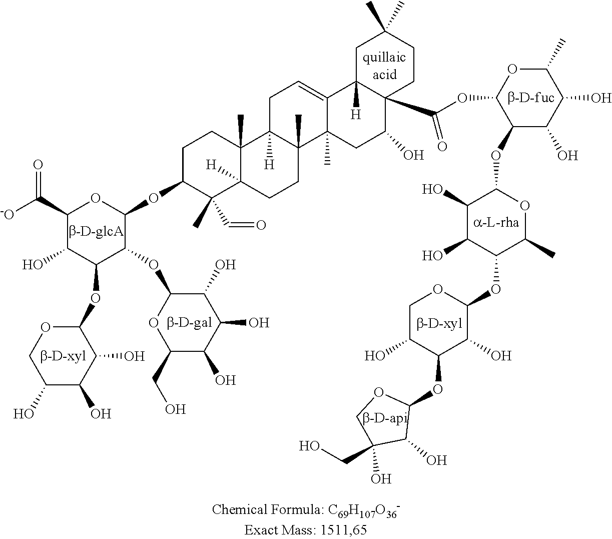

QA-C3-GlcA-Gal-Rha-C28-Fuc-Rha-
Xyl-Xyl-C9
(or QA-C3-GGR-C28-FRXX-C9)

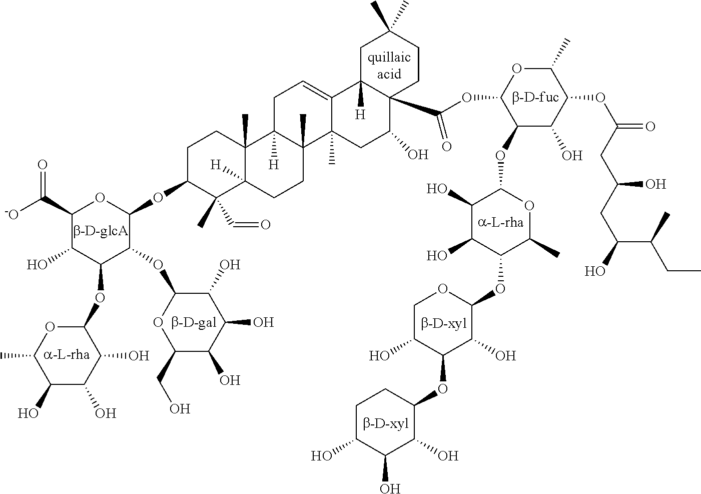

Chemical Formula: C₇₉H₁₂₅O₃₉⁻ Exact Mass: 1697,78

QA-C3-GlcA-Gal-Xyl-C28-Fuc-Rha-
Xyl-Xyl-C9
(or QA-C3-GGX-C28-FRXX-C9)

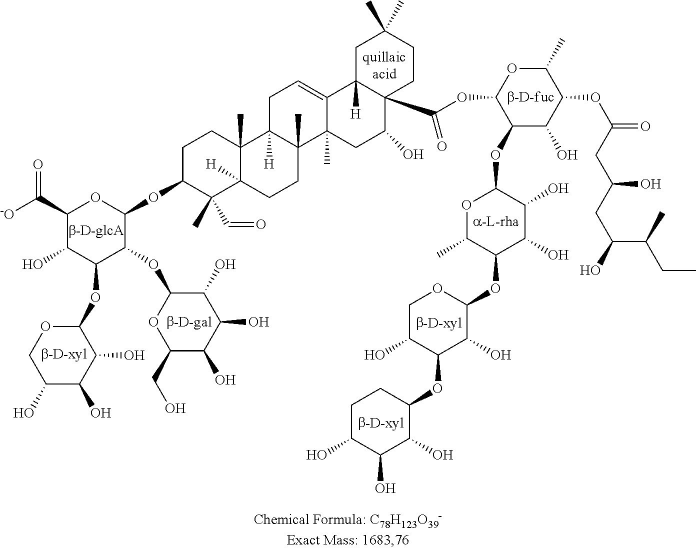

QA-C3-GlcA-Gal-Rha-C28-Fuc-Rha-
Xyl-Api-C9
(or QA-C3-GGR-C28-FRXA-C9)

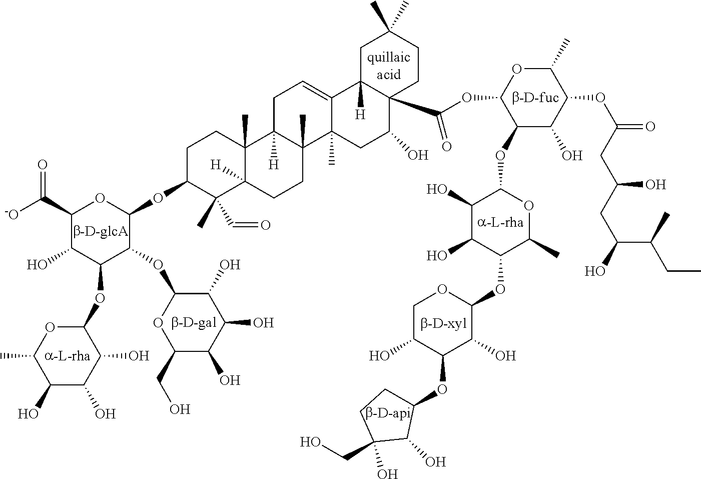

Chemical Formula: C₇₉H₁₂₅O₃₉⁻ Exact Mass: 1697,78

QA-C3-GlcA-Gal-Xyl-C28-Fuc-Rha-
Xyl-Api-C9
(or QA-C3-GGX-C28-FRXA-C9)

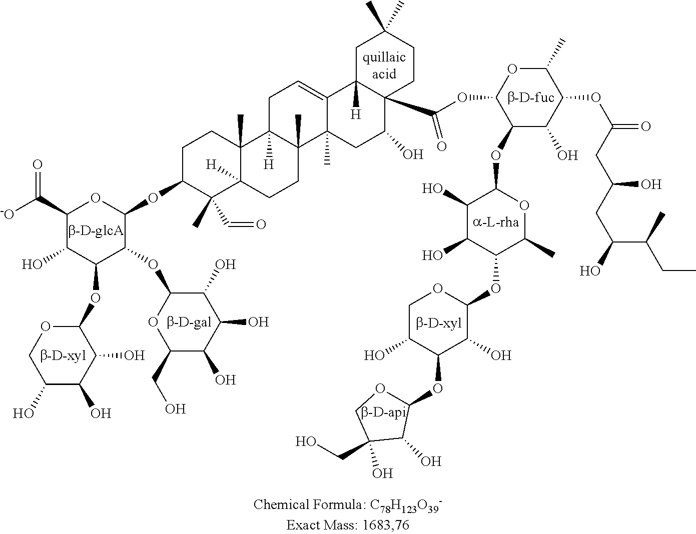

QA-C3-GlcA-Gal-Xyl-C28-Fuc-Rha-
Xyl-C9
(or QA-C3-GGX-C28-FRX-C9)

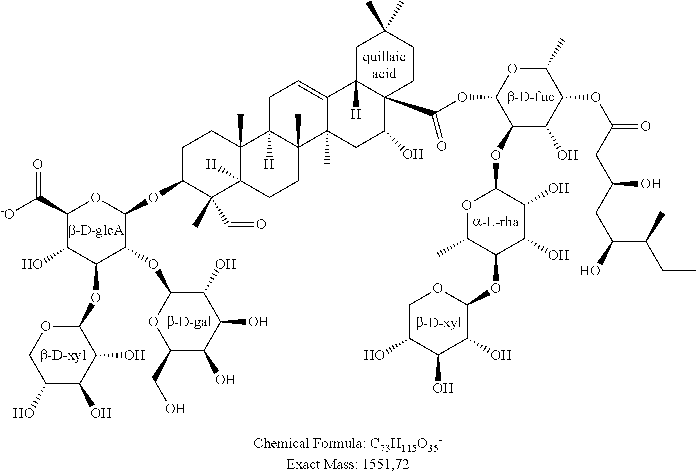

QA-C3-GlcA-Gal-Rha-C28-Fuc-Rha-
Xyl-Xyl-C18
(or QA-C3-GGR-C28-FRXX-C18)

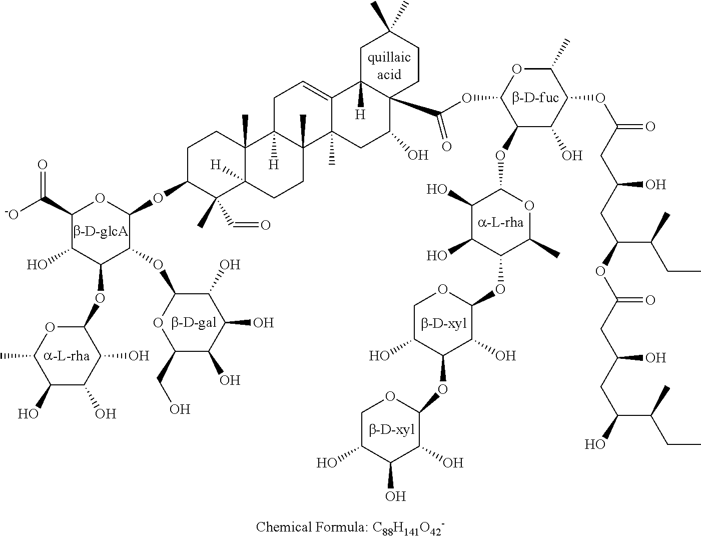

| | |
|---|---|
| QA-C3-GlcA-Gal-Xyl-C28-Fuc-Rha- | (2S,3S,4S,5R,6R)-6- |
| Xyl-Xyl-C18 | (((3S,4S,6aR,6bS,8R,8aR,12aS,14bR)-8a- |
| (or QA-C3-GGX-C28-FRXX-C18) | ((((2S,3R,4S,5R,6R)-3-(((2S,3R,5R,6S)-5-(((2S,4S,5R)-3,5- |
| | dihydroxy-4-(((2S,4S,5R)-3,4,5-trihydroxytetrahydro-2H- |
| | pyran-2-yl)oxy)tetrahydro-2H-pyran-2-yl)oxy)-3,4-dihydroxy- |
| | 6-methyltetrahydro-2H-pyran-2-yl)oxy)-5-(((3R,6R)-5- |
| | (((3R,6R)-5-(((2S,3S,4S,5R)-3,4-dihydroxy-5- |
| | (hydroxymethyl)tetrahydrofuran-2-yl)oxy)-3-hydroxy-6- |
| | methyloctanoyl)oxy)-3-hydroxy-6-methyloctanoyl)oxy)-4- |
| | hydroxy-6-methyltetrahydro-2H-pyran-2-yl)oxy)carbonyl)-4- |
| | formyl-8-hydroxy-4,6a,6b, 11,11,12a,14b-heptamethyl- |
| | 1,2,3,4,4a,5,6,6a,6b,7,8,8a,9,10,11,12,12a,14,14a,14b- |
| | icosahydropicen-3-yl)oxy)-3-hydroxy-5-(((2S,3R,4S,5R,6R)- |
| | 3,4,5-trihydroxy-6-(hydroxymethyl)tetrahydro-2H-pyran-2- |
| | yl)oxy)-4-(((2S,3R,4S,5R)-3,4,5-trihydroxytetrahydro-2H- |
| | pyran-2-yl)oxy)tetrahydro-2H-pyran-2-carboxylic acid |

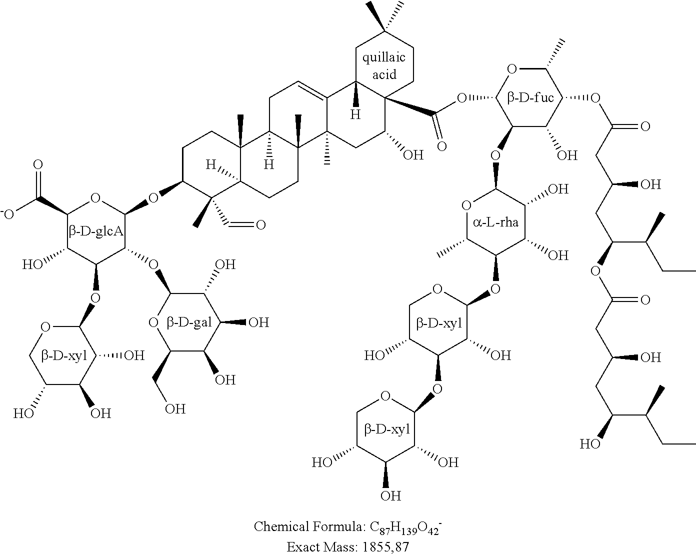

| | |
|---|---|
| QA-C3-GlcA-Gal-Rha-C28-Fuc-Rha- | (2S,3S,4S,5R,6R)-6- |
| Xyl-Api-C18 | (((3S,4S,6aR,6bS,8R,8aR,12aS,14bR)-8a- |
| (or QA-C3-GGR-C28-FRXA-C18) | ((((2S,3R,4S,5R,6R)-3-(((2S,3R,4S,5R,6S)-5- |
| | (((2S,3R,4S,5R)-4-(((2S,3R,4R)-3,4-dihydroxy-4- |
| | (hydroxymethyl)tetrahydrofuran-2-yl)oxy)-3,5- |
| | dihydroxytetrahydro-2H-pyran-2-yl)oxy)-3,4-dihydroxy-6- |
| | methyltetrahydro-2H-pyran-2-yl)oxy)-5-(((3S,6S)-5- |
| | (((3S,6S)-5-(((2R,3R,4R,5S)-3,4-dihydroxy-5- |
| | (hydroxymethyl)tetrahydrofuran-2-yl)oxy)-3-hydroxy-6- |
| | methyloctanoyl)oxy)-3-hydroxy-6-methyloctanoyl) oxy)-4- |
| | hydroxy-6-methyltetrahydro-2H-pyran-2-yl)oxy)carbonyl)-4- |
| | formyl-8-hydroxy-4,6a,6b, 11, 11, 14b-hexamethyl- |
| | 1,2,3,4,4a,5,6,6a,6b,7,8,8a,9,10,11,12,12a,14,14a,14b- |
| | icosahydropicen-3-yl)oxy)-3-hydroxy-5-(((2S,3R,4S,5R,6R)- |
| | 3,4,5-trihydroxy-6-(hydroxymethyl)tetrahydro-2H-pyran-2- |
| | yl)oxy)-4-(((2S,3R,4R,5R,6S)-3,4,5-trihydroxy-6- |
| | methyltetrahydro-2H-pyran-2-yl)oxy)tetrahydro-2H-pyran-2- |
| | carboxylic acid |

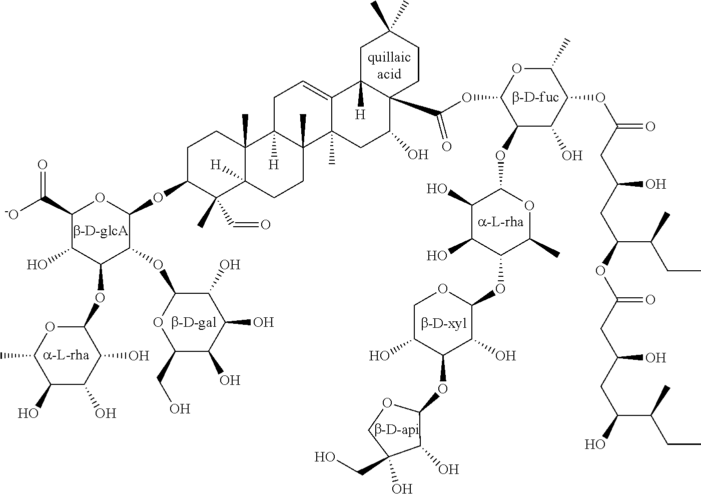

Chemical Formula: C₈₈H₁₄₁O₄₂⁻ Exact Mass: 1869,89

| | |
|---|---|
| QA-C3-GlcA-Gal-Xyl-C28-Fuc-Rha- | (2S,3S,4S,5R)-6-(((3S,4S,6aR,6bS,8R,8aR,12aS,14bR)-8a- |
| Xyl-Api-C18 | ((((2R,3S,4R,5S,6S)-3-(((2S,3R,4S,5R,6S)-5- |
| (or QA-C3-GGX-C28-FRXA-C18) | (((2S,3R,4S,5R)-3,5-dihydroxy-4-(((2S,3R,4R)-4-hydroxy-4- |
| | (hydroxymethyl)-3-methyltetrahydrofuran-2- |
| | yl)oxy)tetrahydro-2H-pyran-2-yl)oxy)-3,4-dihydroxy-6- |
| | methyltetrahydro-2H-pyran-2-yl)oxy)-5-(((6S)-5-(((6S)-5- |
| | (((2R,3R,4R,5S)-3,4-dihydroxy-5- |
| | (hydroxymethyl)tetrahydrofuran-2-yl)oxy)-3-hydroxy-6- |
| | methyloctanoyl)oxy)-3-hydroxy-6-methyloctanoyl) oxy)-4- |
| | hydroxy-6-methyltetrahydro-2H-pyran-2-yl)oxy)carbonyl)-4- |
| | formyl-8-hydroxy-4,6a,6b,11,11,14b-hexamethyl- |
| | 1,2,3,4,4a,5,6,6a,6b,7,8,8a,9,10,11,12,12a,14,14a,14b- |
| | icosahydropicen-3-yl)oxy)-3-hydroxy-5-(((2S,3R,4S,5R,6R)- |
| | 3,4,5-trihydroxy-6-(hydroxymethyl)tetrahydro-2H-pyran-2- |
| | yl)oxy)-4-(((2R,3R,4S,5R)-3,4,5-trihydroxytetrahydro-2H- |
| | pyran-2-yl)oxy)tetrahydro-2H-pyran-2-carboxylic acid |

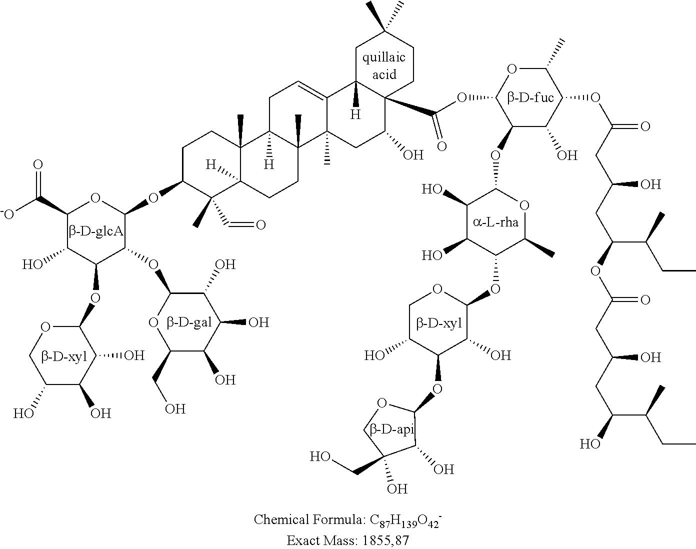

| | |
|---|---|
| QA-C3-GlcA-Gal-Xyl-C28-Fuc-Rha- | (2S,3S,4S,5R)-6-(((3S,4S,6aR,6bS,8R,8aR,12aS,14bR)-8a- |
| Xyl-C18 | ((((2R,3S,4R,5S,6S)-5-(((6S)-5-(((6S)-5-(((2R,3R,4R,5S)- |
| (or QA-C3-GGX-C28-FRX-C18) | 3,4-dihydroxy-5-(hydroxymethyl)tetrahydrofuran-2-yl)oxy)-3- |
| | hydroxy-6-methyloctanoyl)oxy)-3-hydroxy-6- |
| | methyloctanoyl)oxy)-3-(((2S,3R,4S,5R,6S)-3,4-dihydroxy-6- |
| | methyl-5-(((2S,3R,4S,5R)-3,4,5-trihydroxytetrahydro-2H- |
| | pyran-2-yl)oxy)tetrahydro-2H-pyran-2-yl)oxy)-4-hydroxy-6- |
| | methyltetrahydro-2H-pyran-2-yl)oxy)carbonyl)-4-formyl-8- |
| | hydroxy-4,6a,6b,11,11,14b-hexamethyl- |
| | 1,2,3,4,4a,5,6,6a,6b,7,8,8a,9,10,11,12,12a,14,14a,14b- |
| | icosahydropicen-3-yl)oxy)-3-hydroxy-5-(((2S,3R,4S,5R,6R)- |
| | 3,4,5-trihydroxy-6-(hydroxymethyl)tetrahydro-2H-pyran-2- |
| | yl)oxy)-4-(((2R,3R,4S,5R)-3,4,5-trihydroxytetrahydro-2H- |
| | pyran-2-yl)oxy)tetrahydro-2H-pyran-2-carboxylic acid |

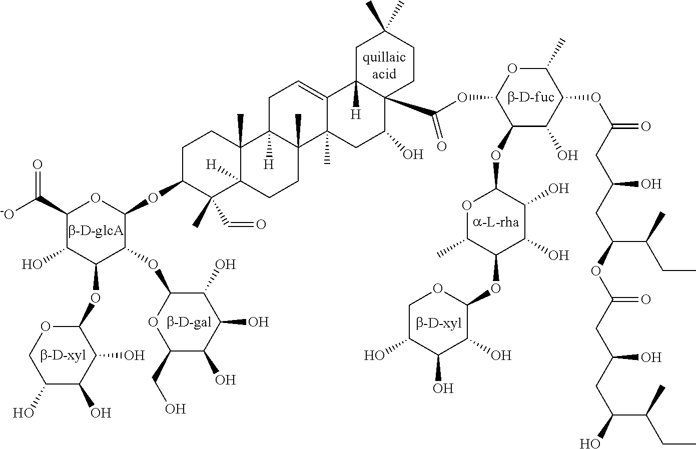

Chemical Formula: C₈₂H₁₃₁O₃₈⁻ Exact Mass: 1723,83

QA-C3-GlcA-Gal-Rham-C28-Fuc-
Rha-Xyl-Xyl-C18-Araf
(or QA-C3-GGR-C28-FRXX-C18-A)

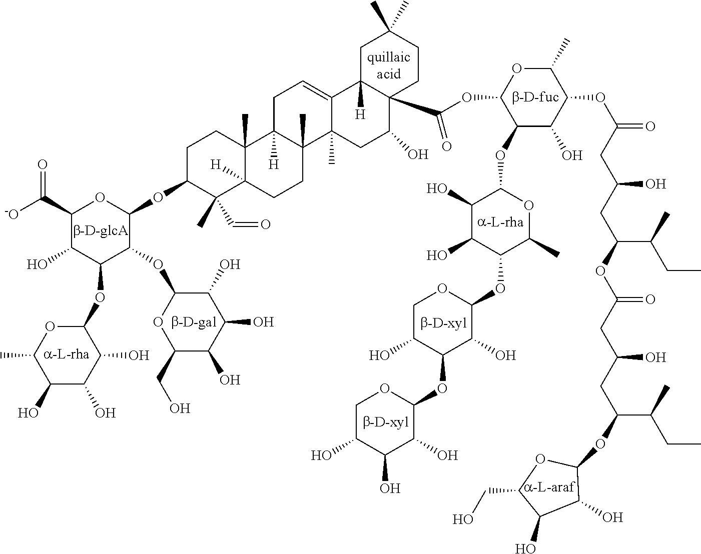

Chemical Formula: C₉₃H₁₄₉O₄₆⁻ Exact Mass: 1723,83

| | |
|---|---|
| QA-C3-GlcA-Gal-Xyl-C28-Fuc-Rha- | (2S,3S,4S,5R,6R)-6- |
| Xyl-Xyl-C18-Araf | (((3S,4S,6aR,6bS,8R,8aR,12aS,14bR)-8a- |
| (or QA-C3-GGX-C28-FRXX-C18-A) | ((((2S,3R,4S,5R,6R)-3-(((2S,3R,5R,6S)-5-(((2S,4S,5R)-3,5- |
| (or QS-21-Xyl) | dihydroxy-4-(((2S,4S,5R)-3,4,5-trihydroxytetrahydro-2H- |
| | pyran-2-yl)oxy)tetrahydro-2H-pyran-2-yl)oxy)-3,4-dihydroxy- |
| | 6-methyltetrahydro-2H-pyran-2-yl)oxy)-5-(((3R,6R)-5- |
| | (((3R,6R)-5-(((2S,3S,4S,5R)-3,4-dihydroxy-5- |
| | (hydroxymethyl)tetrahydrofuran-2-yl)oxy)-3-hydroxy-6- |
| | methyloctanoyl)oxy)-3-hydroxy-6-methyloctanoyl) oxy)-4- |
| | hydroxy-6-methyltetrahydro-2H-pyran-2-yl)oxy)carbonyl)-4- |
| | formyl-8-hydroxy-4,6a,6b,11,11,12a,14b-heptamethyl- |
| | 1,2,3,4,4a,5,6,6a,6b,7,8,8a,9,10,11,12,12a,14,14a,14b- |
| | icosahydropicen-3-yl)oxy)-3-hydroxy-5-(((2S,3R,4S,5R,6R)- |
| | 3,4,5-trihydroxy-6-(hydroxymethyl)tetrahydro-2H-pyran-2- |
| | yl)oxy)-4-(((2S,3R,4S,5R)-3,4,5-trihydroxytetrahydro-2H- |
| | pyran-2-yl)oxy)tetrahydro-2H-pyran-2-carboxylic acid |

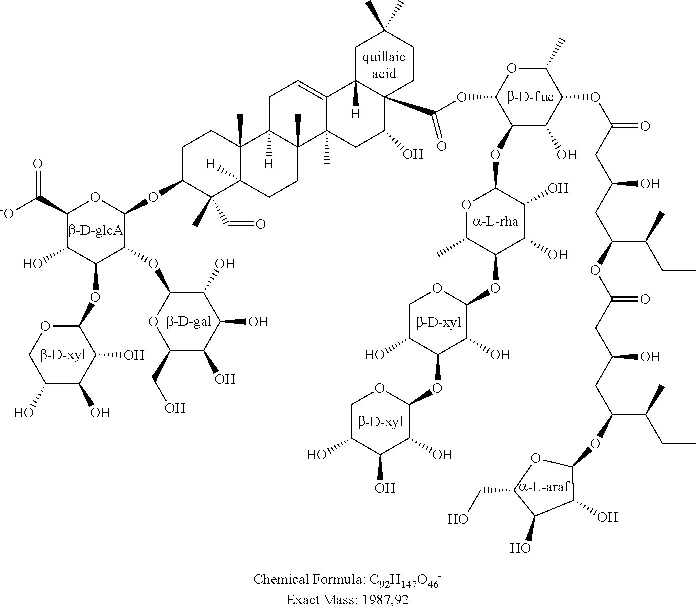

| | |
|---|---|
| QA-C3-GlcA-Gal-Rha-C28-Fuc-Rha- | (2S,3S,4S,5R,6R)-6- |
| Xyl-Api-C18-Araf | (((3S,4S,6aR,6bS,8R,8aR,12aS,14bR)-8a- |
| (or QA-C3-GGR-C28-FRXA-C18-A) | ((((2S,3R,4S,5R,6R)-3-(((2S,3R,4S,5R,6S)-5- |
| | (((2S,3R,4S,5R)-4-(((2S,3R,4R)-3,4-dihydroxy-4- |
| | (hydroxymethyl)tetrahydrofuran-2-yl)oxy)-3,5- |
| | dihydroxytetrahydro-2H-pyran-2-yl)oxy)-3,4-dihydroxy-6- |
| | methyltetrahydro-2H-pyran-2-yl)oxy)-5-(((3S,6S)-5- |
| | (((3S,6S)-5-(((2R,3R,4R,5S)-3,4-dihydroxy-5- |
| | (hydroxymethyl)tetrahydrofuran-2-yl)oxy)-3-hydroxy-6- |
| | methyloctanoyl)oxy)-3-hydroxy-6-methyloctanoyl)oxy)-4- |
| | hydroxy-6-methyltetrahydro-2H-pyran-2-yl)oxy)carbonyl)-4- |
| | formyl-8-hydroxy-4,6a,6b,11,11,14b-hexamethyl- |
| | 1,2,3,4,4a,5,6,6a,6b,7,8,8a,9,10,11,12,12a,14,14a,14b- |
| | icosahydropicen-3-yl)oxy)-3-hydroxy-5-(((2S,3R,4S,5R,6R)- |
| | 3,4,5-trihydroxy-6-(hydroxymethyl)tetrahydro-2H-pyran-2- |
| | yl)oxy)-4-(((2S,3R,4R,5R,6S)-3,4,5-trihydroxy-6- |
| | methyltetrahydro-2H-pyran-2-yl)oxy)tetrahydro-2H-pyran-2- |
| | carboxylic acid |

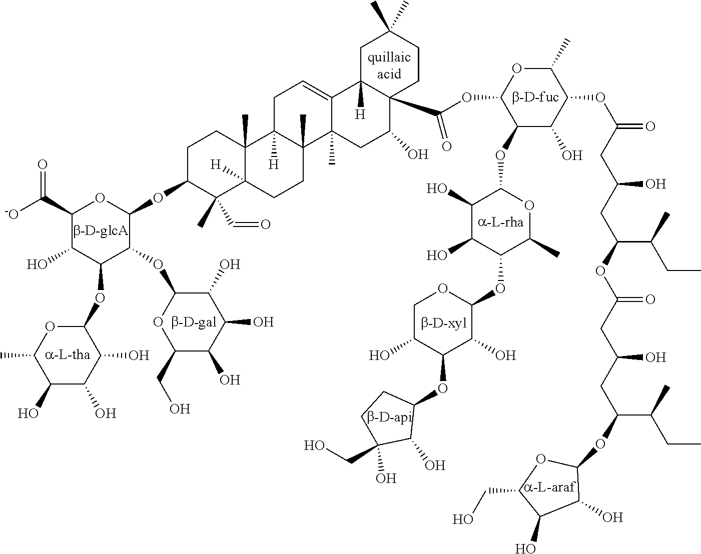

Chemical Formula: C₉₃H₁₄₉O₄₆⁻ Exact Mass: 2001,93

| | |
|---|---|
| QA-C3-GlcA-Gal-Xyl-C28-Fuc-Rha- | (2S,3S,4S,5R)-6-(((3S,4S,6aR,6bS,8R,8aR,12aS,14bR)-8a- |
| Xyl-Api-C18-Araf | ((((2R,3S,4R,5S,6S)-3-(((2S,3R,4S,5R,6S)-5- |
| (or QA-C3-GGX-C28-FRXA-C18-A) | (((2S,3R,4S,5R)-3,5-dihydroxy-4-(((2S,3R,4R)-4-hydroxy-4- |
| (or QS-21-Api) | (hydroxymethyl)-3-methyltetrahydrofuran-2- |
| | yl)oxy)tetrahydro-2H-pyran-2-yl)oxy)-3,4-dihydroxy-6- |
| | methyltetrahydro-2H-pyran-2-yl)oxy)-5-(((6S)-5-(((6S)-5- |
| | (((2R,3R,4R,5S)-3,4-dihydroxy-5- |
| | (hydroxymethyl)tetrahydrofuran-2-yl)oxy)-3-hydroxy-6- |
| | methyloctanoyl)oxy)-3-hydroxy-6-methyloctanoyl)oxy)-4- |
| | hydroxy-6-methyltetrahydro-2H-pyran-2-yl)oxy)carbonyl)-4- |
| | formyl-8-hydroxy-4,6a,6b,11,11,14b-hexamethyl- |
| | 1,2,3,4,4a,5,6,6a,6b,7,8,8a,9,10,11,12,12a,14,14a,14b- |
| | icosahydropicen-3-yl)oxy)-3-hydroxy-5-(((2S,3R,4S,5R,6R)- |
| | 3,4,5-trihydroxy-6-(hydroxymethyl)tetrahydro-2H-pyran-2- |
| | yl)oxy)-4-(((2R,3R,4S,5R)-3,4,5-trihydroxytetrahydro-2H- |
| | pyran-2-yl)oxy)tetrahydro-2H-pyran-2-carboxylic acid |

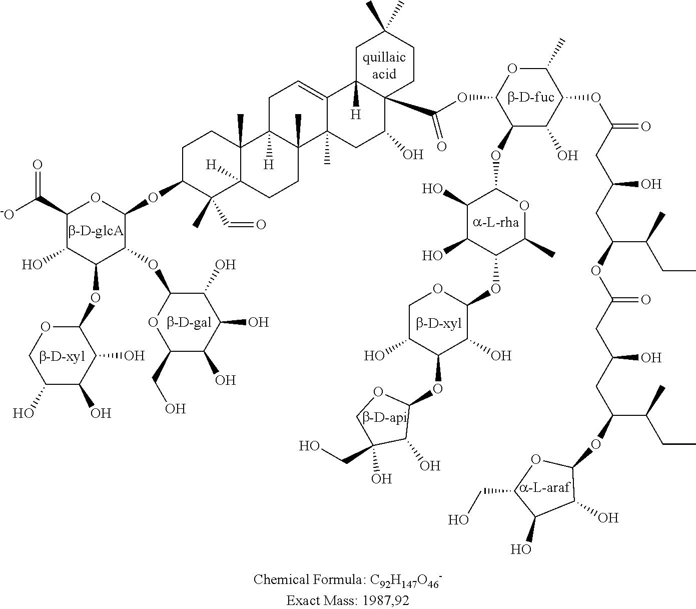

| | |
|---|---|
| QA-C3-GlcA-Gal-Xyl-C28-Fuc-Rha- | (2S,3S,4S,5R)-6-(((3S,4S,6aR,6bS,8R,8aR,12aS,14bR)-8a- |
| Xyl-C18-Araf | ((((2R,3S,4R,5S,6S)-5-(((6S)-5-(((6S)-5-(((2R,3R,4R,5S)- |
| (or QA-C3-GGX-C28-FRX-C18-Araf) | 3,4-dihydroxy-5-(hydroxymethyl)tetrahydrofuran-2-yl)oxy)-3- |
| | hydroxy-6-methyloctanoyl)oxy)-3-hydroxy-6- |
| | methyloctanoyl)oxy)-3-(((2S,3R,4S,5R,6S)-3,4-dihydroxy-6- |
| | methyl-5-(((2S,3R,4S,5R)-3,4,5-trihydroxytetrahydro-2H- |
| | pyran-2-yl)oxy)tetrahydro-2H-pyran-2-yl)oxy)-4-hydroxy-6- |
| | methyltetrahydro-2H-pyran-2-yl)oxy)carbonyl)-4-formyl-8- |
| | hydroxy-4,6a,6b,11,11,14b-hexamethyl- |
| | 1,2,3,4,4a,5,6,6a,6b,7,8,8a,9,10,11,12,12a,14,14a,14b- |
| | icosahydropicen-3-yl)oxy)-3-hydroxy-5-(((2S,3R,4S,5R,6R)- |
| | 3,4,5-trihydroxy-6-(hydroxymethyl)tetrahydro-2H-pyran-2- |
| | yl)oxy)-4-(((2R,3R,4S,5R)-3,4,5-trihydroxytetrahydro-2H- |
| | pyran-2-yl)oxy)tetrahydro-2H-pyran-2-carboxylic acid |

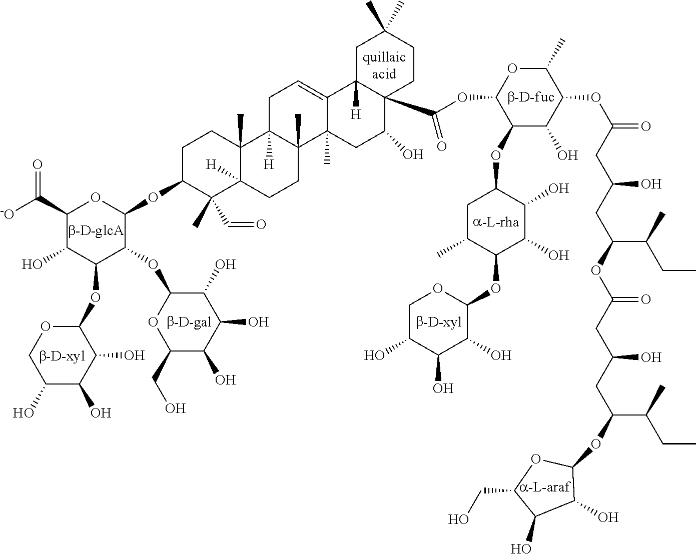

Chemical Formula: C87H139O42- Exact Mass: 1855,87

QA-C3-GlcA-Gal-Xyl-C28-Fuc-Rha-
Xyl-C18-Xyl
(or QA-C3-GGX-C28-FRX-C18-Xyl)

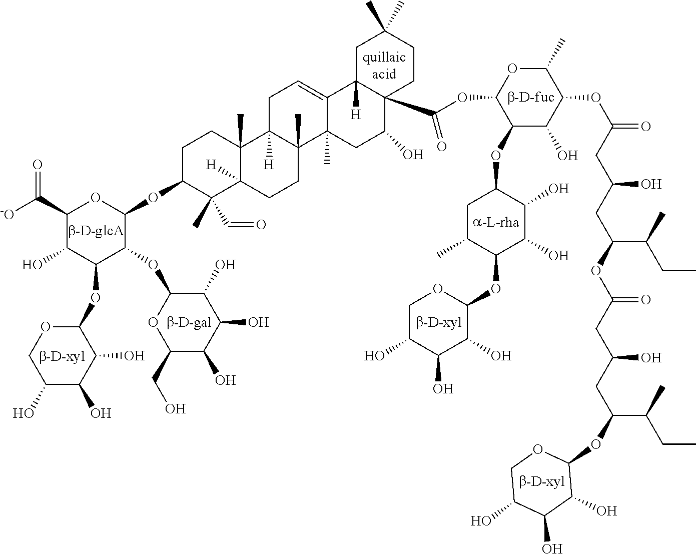

Chemical Formula: C₈₇H₁₃₉O₄₂⁻ Exact Mass: 1855,87

QA-C3-GlcA-Gal-Xyl-C28-Fuc-Rha-
Xyl-Xyl-C18-Xyl
(or QA-C3-GGX-C28-FRXX-C18-Xyl)

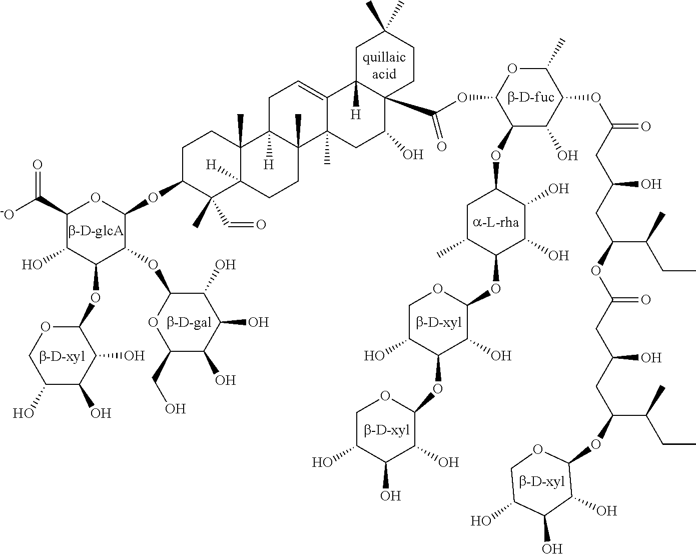

Chemical Formula: C₉₂H₁₄₇O₄₆⁻ Exact Mass: 1987,92

QA-C3-GlcA-Gal-Xyl-C28-Fuc-Rha-
Xyl-Api-C18-Xyl
(or QA-C3-GGX-C28-FRXA-C18-Xyl)

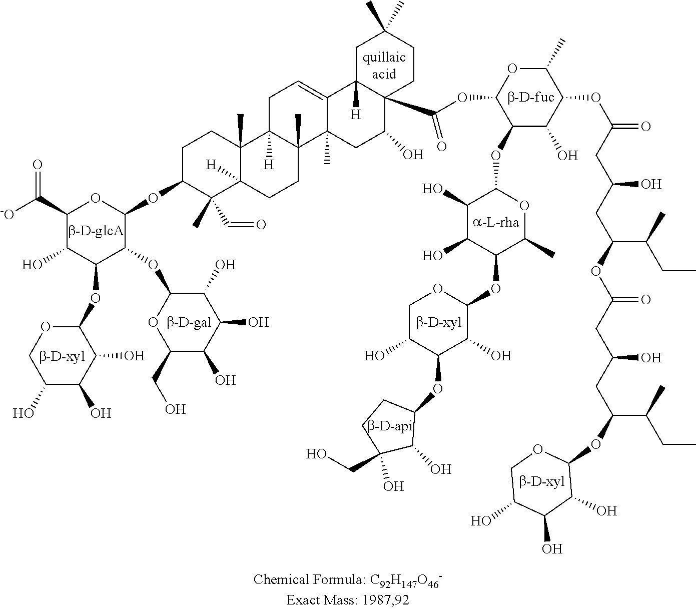

Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in or “approximately” in relation to a numerical value x is optional and means, for example, x±10% of the given figure, such as x+5% of the given figure, in particular the given figure.

As used herein, the singular forms “a,” “an” and “the” include plural references unless the content clearly dictates otherwise.

As used herein, ng refers to nanograms, ug or μg refers to micrograms, mg refers to milligrams, mL or ml refers to milliliter, and mM refers to millimolar. Similar terms, such as um, are to be construed accordingly.

Unless specifically stated, a process comprising a step of mixing two or more components does not require any specific order of mixing. Thus components can be mixed in any order. Where there are three components then two components can be combined with each other, and then the combination may be combined with the third component, etc.

The invention is illustrated further by reference to the following clauses.

A method of producing quillaic acid (QA) in yeast, wherein the method comprises the step of overexpressing, in a yeast engineered to produce β-amyrin, heterologous genes encoding the following enzymes:
a cytochrome P450 C16 oxidase, wherein the C16 oxidase oxidizes the C16 carbon of β-amyrin to a hydroxyl group,
a cytochrome P450 C23 oxidase, wherein the C23 oxidase oxidizes the C23 carbon of β-amyrin to an aldehyde group,
a cytochrome P450 C28 oxidase, wherein the C28 oxidase oxidizes the C28 carbon of β-amyrin to a carboxyl group, and
a cytochrome P450 reductase (CPR), acting as a redox partner
wherein the C16 oxidase, the C23 oxidase, the C28 oxidase and the CPR are from a plant origin.

The method of clause 1, wherein the C16 oxidase, the C23 oxidase and the C28 oxidase are independently selected from Artemisia annua (Aa), Arabidopsis thaliana (At), Glycyrrhiza glabra (Gg), Medicago truncatula (Mt), Quillaja saponaria (Qs), Saponaria vaccaria (Sv), Centella asiatica (Ca), Bupleurum falcatum (Bf) and Maesa lanceolate (MI).

The method of clause 1 or clause 2, wherein the C16 oxidase, the C23 oxidase and the C28 oxidase are independently selected from Medicago truncatula (Mt), Bupleurum falcatum (Bf), Quillaja saponaria (Qs), and Saponaria vaccaria (Sv).

The method of clause 3, wherein the C16 oxidase is selected from QsC16 according to SEQ ID NO: 20, QsC28C16 according to SEQ ID NO: 23, and SvC16 according to SEQ ID NO: 26.

The method of clause 4, wherein QsC16 is encoded by the nucleotide sequence SEQ ID NO: 21, QsC28C16 is encoded by the nucleotide sequence SEQ ID NO: 24 and SvC16 is encoded by the nucleotide sequence SEQ ID NO: 27.

The method of any one of clauses 1 to 5, wherein the C23 oxidase is selected from MtC23 oxidase according to SEQ ID NO: 38, QsC23 according to SEQ ID NO: 29, SvC23-1 according to SEQ ID NO: 32, and SvC23-2 according to SEQ ID NO: 35.

The method of clause 6, wherein MtC23 is encoded by the nucleotide sequence SEQ ID NO: 39, QsC23 is encoded by the nucleotide sequence SEQ ID NO: 30, SvC23-1 is encoded by the nucleotide sequence SEQ ID NO: 33, and SvC23-2 is encoded by the nucleotide sequence SEQ ID NO: 36.

The method of any one of clauses 1 to 7, wherein the C28 oxidase is selected from MtC28 according to SEQ ID NO: 46, QsC28 according to SEQ ID NO: 41 and SvC28 according to SEQ ID NO: 44.

The method of clause 8, wherein MtC28 is encoded by the nucleotide sequence SEQ ID NO: 47, QsC28 is encoded by the nucleotide sequence SEQ ID NO: 42, and SvC28 is encoded by the nucleotide sequence SEQ ID NO: 45.

The method of any one of clauses 4 to 9, wherein the C16 oxidase is SvC16 according to SEQ ID NO: 26, the C23 oxidase is SvC23-1 according to SEQ ID NO: 32 or SvC23-2 oxidase according to SEQ ID NO: 35, and the C28 oxidase is SvC28 according to SEQ ID NO: 44.

The method of clause 10, wherein SvC16 is encoded by the nucleotide sequence SEQ ID NO: 27, SvC23-1 is encoded by the nucleotide sequence SEQ ID NO: 33, SvC23-2 is encoded by the nucleotide sequence SEQ ID NO: 36, and SvC28 is encoded by the nucleotide sequence SEQ ID NO: 45.

The method of any one of clauses 4 to 9, wherein the C16 oxidase is selected from QsC16 according to SEQ ID NO: 20 and QsC28C16 according to SEQ ID NO: 23, the C23 oxidase is QsC23 according to SEQ ID NO: 29 and the C28 is QsC28 according to SEQ ID NO: 41.

The method of clause 12, wherein QsC16 is encoded by the nucleotide sequence SEQ ID NO: 21, QsC28C16 is encoded by the nucleotide sequence SEQ ID NO: 24, QsC23 is encoded by the nucleotide sequence SEQ ID NO: 30 and QsC28 is encoded by the nucleotide sequence SEQ ID NO: 42.

The method of any one of clauses 4 to 9, wherein the C16 oxidase is QsC28C16 according to SEQ ID NO: 23, the C23 oxidase is QsC23 according to SEQ ID NO: 29, and the C28 oxidase is QsC28 according to SEQ ID NO: 41.

The method of any one of clause 14, wherein the QsC28C16 is encoded by the nucleotide sequence SEQ ID NO: 24, QsC23 is encoded by the nucleotide sequence SEQ ID NO: 30, and QsC28 is encoded by the nucleotide sequence SEQ ID NO: 42.

The method of any one of clauses 1 to 15, wherein the CPR is selected from A. thaliana (At) and Lotus Japonicus (Lj).

The method of clause 16, wherein the CPR is selected from AtATR1 according to SEQ ID NO: 49 and LjCPR according to SEQ ID NO: 52.

The method of clause 17, wherein the CPR is AtATR1 according to SEQ ID NO: 49.

The method of clause 17 or clause 18, wherein AtATR1 is encoded by the nucleotide sequence SEQ ID NO: 50 and LjCPR is encoded by the nucleotide sequence SEQ ID NO: 53.

The method of any one of clauses 1 to 19, wherein the yeast further overexpresses a heterologous gene encoding (v) a cytochrome b5.

The method of clause 20, wherein the cytochrome b5 is selected from A. thaliana (At), Q. saponaria (Qs) and S. vaccaria (Sv).

The method of clause 21, wherein the cytochrome b5 is selected from Atb5 according to SEQ ID NO: 58, Qsb5 according to SEQ ID NO: 55 and Svb5 according to SEQ ID NO: 61.

The method of clause 21 or clause 22, wherein the cytochrome b5 is Qsb5 according to SEQ ID NO: 55.

The method of clause 21 or clause 22, wherein the cytochrome b5 is Svb5 according to SEQ ID NO: 61.

The method of any one of clauses 22 to 24, wherein Atb5 is encoded by the nucleotide sequence SEQ ID NO: 59, Qsb5 is encoded by the nucleotide sequence SEQ ID NO: 56, and Svb5 is encoded by the nucleotide sequence SEQ ID NO: 62.

The method of any one of clauses 1 to 25, wherein the yeast further overexpresses a heterologous gene encoding (vi) a scaffold protein, wherein the scaffold protein physically interacts with one or more of the C16 oxidase, the C23 oxidase, the C28 oxidase and the CPR.

The method of clause 26, wherein the scaffold protein is a membrane steroid-binding protein (MSBP).

The method of clause 27, wherein the MSBP is selected from A. thaliana (At), Q. Saponaria (Qs) and S. vaccaria (Sv).

The method of clause 27 or clause 28, wherein the MSBP is selected from AtMSBP1 according to SEQ ID NO: 63 and AtMSBP2 according to SEQ ID NO: 65.

The method of clause 27 or clause 28, wherein the MSBP is selected from QsMSBP1 according to SEQ ID NO: 73, SvMSBP1 according to SEQ ID NO: 67 and SvMSBP2 according to SEQ ID NO: 70.

The method of clause 27, clause 28 or clause 30, wherein the MSBP is SvMSBP1 according to SEQ ID NO: 67.

The method of any one of clauses 29 to 31, wherein AtMSBP1 is encoded by the nucleotide sequence SEQ ID NO: 64, AtMSBP2 is encoded by the nucleotide sequence SEQ ID NO: 66, QsMSBP1 is encoded by the nucleotide sequence SEQ ID NO: 74, SvMSBP1 is encoded by the nucleotide sequence SEQ ID NO: 68 and SvMSBP2 is encoded by the nucleotide sequence SEQ ID NO: 71.

The method of any one of clauses 1 to 32, wherein the yeast engineered to produce β-amyrin overexpresses a β-amyrin synthase (BAS) selected from A. annua (Aa), A. thaliana (At), G. glabra (Gg), G. vaccaria (Gv), S. vaccaria (Sv), and Q. saponaria (Qs).

The method of clause 33, wherein the BAS is selected from AaBAS according to SEQ ID NO: 1, AtBAS according to SEQ ID NO: 4, GgBAS according to SEQ ID NO: 7, GvBAS according to SEQ ID NO: 10, QsBAS according to SEQ ID NO: 15, and SvBAS according to SEQ ID NO: 13.

The method of clause 33 or clause 34, wherein the BAS is GvBAS according to SEQ ID NO: 10.

The method of any one of clauses 34 to 35, wherein AaBAS is encoded by the nucleotide sequence SEQ ID NO: 2, AtBAS is encoded by the nucleotide sequence SEQ ID NO: 5, GgBAS is encoded by the nucleotide sequence SEQ ID NO: 8, GvBAS is encoded by the nucleotide sequence SEQ ID NO: 11, QsBAS is encoded by the nucleotide sequence SEQ ID NO: 16, and SvBAS is encoded by the nucleotide sequence SEQ ID NO: 14.

The method of any one of clauses 1 to 9, 12 to 23, 25 to 28, and 30 to 36, wherein the C16 oxidase is QsC28C16, the C23 oxidase is QsC23, the C28 oxidase is QsC28, the CPR is AtATR1, the MSBP is SvMSBP1, the cytochrome b5 is Qsb5, and the BAS is GvBAS.

The method of clause 37, wherein QsC28C16 is encoded by the nucleotide sequence SEQ ID NO: 24, QsC23 is encoded by the nucleotide sequence SEQ ID NO: 30, QsC28 is encoded by the nucleotide sequence SEQ ID NO: 42, AtATR1 is encoded by the nucleotide sequence SEQ ID NO: 24, SvMSBP1 is encoded by the nucleotide sequence SEQ ID NO: 50, Qsb5 is encoded by the nucleotide sequence SEQ ID NO: 56, and GvBAS is encoded by the nucleotide sequence SEQ ID NO: 11.

A yeast which is engineered to produce QA according to the method of any one of clauses 1 to 38.

The yeast of clause 39 producing at least 60 mg/L of QA.

A method of producing UDP-Glucuronic acid (UDP-GlcA) in yeast, wherein the method comprises the step of overexpressing a heterologous gene encoding a UDP-glucose dehydrogenase (UGD) converting UDP-Glucose (UDP-Glc) into UDP-GlcA.

The method of clause 41, wherein the UGD is from A. thaliana (At).

The method of clause 39, wherein the UGD is selected from AtUGD according to SEQ ID NO: 84 and AtUGD_{A101L} according to SEQ ID NO: 108.

The method of any one of clauses 41 to 43, wherein the UGD is AtUGD_{A101L} according to SEQ ID NO: 108.

The method of clause 43 or clause 44, wherein AtUGD is encoded by the nucleotide sequence SEQ ID NO: 85, and AtUGD_{A101L} is encoded by the nucleotide sequence SEQ ID NO: 109.

A yeast which is engineered to produce UDP-GlcA according to the method of any of clauses 41 to clause 45.

A method of producing UDP-Rhamnose (UDP-Rha) in yeast, wherein the method comprises the step of overexpressing a heterologous gene encoding a UDP-rhamnose synthase converting UDP-Glucose (UDP-Glc) into UDP-Rha.

The method of clause 47, wherein the UDP-rhamnose synthase is from A. thaliana (At).

The method of clause 48, wherein the UDP-rhamnose synthase is AtRHM2 according to SEQ ID NO: 102.

The method of clause 49, wherein AtRHM2 is encoded by the nucleotide sequence SEQ ID NO: 103.

A yeast which is engineered to produce UDP-Rha according to the method of any one of clauses 47 to 50.

A method of producing UDP-Xylose (UDP-Xyl) in yeast, wherein the method comprises the step of overexpressing heterologous genes encoding the following enzymes:
a UDP-glucose dehydrogenase (UGD) converting UDP-Glc into UDP-GlcA, and
a UDP-xylose synthase (UXS) converting UDP-GlcA into UDP-Xylose.

The method of clause 52, wherein the UGD and the UXS are independently selected from A. thaliana (At) and Q. saponaria (Qs).

The method of clause 53, wherein the UGD is selected from AtUGD according to SEQ ID NO: 84 and AtUGD_{A101L} according to SEQ ID NO: 108.

The method of clause 52, wherein the UGD is selected from Synechococcus sp. (Syn), Homo sapiens (Hs), Paramoeba atlantica (Patl), Bacillus cytotoxicus (Bcyt), Corallococcus macrosporus (Myxfulv), and Pyrococcus furiosus (Pfu).

The method of clause 55, wherein the UGD is selected from SynUGD according to SEQ ID NO: 154, HsUGD_{A104L} according to SEQ ID NO: 157, PatlUGD according to SEQ ID NO: 110, BcytUGD according to SEQ ID NO: 160, MyxfulvUGD according to SEQ ID NO: 163, and PfuUGD according to SEQ ID NO: 166.

The method of any one of clauses 52 to 54, wherein the UGD is AtUGD_{A101L} according to SEQ ID NO: 108.

The method of any of clauses 54 to 57, wherein AtUGD is encoded by the nucleotide sequence SEQ ID NO: 85, AtUGD_{A101L} is encoded by the nucleotide sequence SEQ ID NO: 109, SynUGD is encoded by the nucleotide sequence SEQ ID NO: 155, HsUGD_{104L} is encoded by the nucleotide sequence SEQ ID NO: 158, PatlUGD is encoded by the nucleotide sequence SEQ ID NO: 111, BcytUGD is encoded by the nucleotide sequence SEQ ID NO: 161, MyxfulvUGD is encoded by the nucleotide sequence SEQ ID NO: 164, and PfuUGD is encoded by the nucleotide sequence SEQ ID NO: 167.

The method of any one of clauses 52 to 58, wherein the UXS is selected from AtUXS according to SEQ ID NO: 105 and QsAXS according to SEQ ID NO: 113.

The method of clause 60 wherein the UGD is AtUGD_{A101L} according to SEQ ID NO: 108 and the UXS is AtUXS according to SEQ ID NO: 105.

The method of clause 59 or clause 60, wherein AtUXS is encoded by the nucleotide sequence SEQ ID NO: 106, QsAXS is encoded by the nucleotide sequence SEQ ID NO: 114 and AtUGD_{A101L} is encoded by the nucleotide sequence SEQ ID NO: 109.

A yeast which is engineered to produce UDP-Xyl according to the method of any one of clauses 52 to 61.

A method of producing a C3-glycosylated QA derivative in yeast, wherein the derivative is QA-C3-GlcA, and the method comprises the step of overexpressing, in a yeast engineered to produce QA and UDP-GlcA, a heterologous gene encoding the following enzyme:
a UDP-GlcA transferase (GlcAT) transferring UDP-GlcA and attaching a GlcA residue at the C3 position of QA to form QA-C3-GlcA.

The method of clause 63, wherein the GlcAT is selected from Q. saponaria (Qs) and S. vaccaria (Sv).

The method of clause 63 or clause 64, wherein the GlcAT is from Q. saponaria.

The method of clause 65, wherein the GlcAT is selected from QsCslG1 according to SEQ ID NO: 78 and QsCslG2 according to SEQ ID NO: 81.

The method of clause 66, wherein the GlcAT is QsCslG2 according to SEQ ID NO: 81.

The method of clause 64, wherein the GlcAT is from S. vaccaria.

The method of clause 68, wherein the GlcAT is SvCslG according to SEQ ID NO: 76.

The method of any one of clauses 66, 67, 68 or 69, wherein QsCslG1 is encoded by the nucleotide sequence SEQ ID NO: 79, QsCslG2 is encoded by the nucleotide sequence SEQ ID NO: 82 and SvCslG is encoded by the nucleotide sequence SEQ ID NO: 77.

The method of any one of clauses 63 to 70, wherein the yeast engineered to produce QA is according to clause 39.

The method of clause 71, wherein the yeast engineered to produce UDP-GlcA is according to clause 46.

A yeast which is engineered to produce QA-C3-GlcA according to the method of any one of clauses 63 to 72.

The method of any one of clauses 63 to 72, wherein the derivative is QA-C3-GlcA-Gal, and the overexpressing further comprises overexpressing a heterologous gene encoding the following enzyme:
a UDP-Galactose transferase (GalT) transferring UDP-Gal and attaching a Gal residue to QA-C3-GlcA to form QA-C3-GlcA-Gal.

The method of clause 74, wherein the GalT is selected from Q. saponaria (Qs) and S. vaccaria (Sv).

The method of clause 75, wherein the GalT is from Q. saponaria (Qs).

The method of any one of clause 70 to 76, wherein the GalT is OsGalT according to SEQ ID NO: 116.

The method of clause 74, wherein the GalT is from S. vaccaria.

The method of clause 78, wherein GalT is SvGalT according to SEQ ID NO: 98.

The method of clause 77 or clause 79, wherein QsGalT is encoded by the nucleotide sequence SEQ ID NO: 117 and SvGalT is encoded by the nucleotide sequence SEQ ID NO: 99.

A yeast which is engineered to produce QA-C3-GlcA-Gal according to the method of any one of clauses 74 to 80.

The method of any one of clauses 74 to 80, wherein the derivative is QA-C3-GlcA-Gal-Rha, the yeast is further engineered to produce UDP-Rha, and the overexpressing further comprises overexpressing a heterologous gene encoding the following enzyme:
a UDP-Rhamnose transferase (RhaT) transferring UDP-Rha and attaching a Rha residue to QA-C3-GlcA-Gal to form QA-C3-GlcA-Gal-Rha.

The method of clause 82, wherein the RhaT is from Q. saponaria (Qs).

The method of clause 83, wherein the RhaT is QsRhaT according to SEQ ID NO: 119.

The method of clause 84, wherein QsRhaT is encoded by the nucleotide sequence SEQ ID NO: 120.

The method of any one of clauses 82 to 86, wherein the yeast engineered to produce UDP-Rha is according to clause 51.

A yeast which is engineered to produce QA-C3-GlcA-Gal-Rha according to the method of any one of clauses 82 to 86.

The method of any one of clauses 74 to 80, wherein the derivative is QA-C3-GlcA-Gal-Xyl, the yeast is further engineered to produce UDP-Xyl, and the overexpressing further comprises overexpressing heterologous genes encoding the following enzymes:
a UDP-Xylose transferase (XylT) transferring UDP-Xylose and attaching a Xyl residue to QA-C3-GlcA-Gal to form QA-C3-GlcA-Gal-Xyl.

The method of clause 88, wherein the XylT is selected from Q. Saponaria (Qs) or S. vaccaria (Sv).

The method of clause 89, wherein the XylT is selected from QsC3XylT according to SEQ ID NO: 122 and SvC3XylT according to SEQ ID NO: 100.

The method of clause 90, wherein QsC3XylT is encoded by the nucleotide sequence SEQ ID NO: 123 and SvC3XylT is encoded by the nucleotide sequence SEQ ID NO: 101, and wherein the yeast engineered to produce UDP-Xyl is according to clause 62.

A yeast which is engineered to produce QA-C3-GlcA-Gal-Xyl according to the method of any one of clauses 88 to 91.

The method of any one of clauses 88 to 91, wherein the overexpressing further comprises overexpressing of heterologous genes encoding the following enzymes:
a glucuronokinase (GlcAK) converting free glucuronic acid into GlcA-1-phosphate, and
a UDP-sugar pyrophosphorylase (USP) converting GlcA-1-phosphate into UDP-GlcA,
and glucuronic acid is supplemented exogenously.

The method of clause 93, wherein the GlcAK and the USP are from A. thaliana (At).

The method of clause 94, wherein GlcAK is AtGlcAK according to SEQ ID NO: 169 and the USP is AtUSP according to SEQ ID NO: 223.

The method of clause 95, wherein AtGlcAK is encoded by the nucleotide sequence SEQ ID NO: 170 and AtUSP is encoded by the nucleotide sequence SEQ ID NO: 224.

The method of any one of clauses 93 to 96, wherein the overexpressing further comprises overexpressing of (vi) a heterologous gene encoding a Myo-Inositol Oxygenase (MIOX), and myo-inositol is additionally supplemented exogenously.

The method of clause 97, wherein MIOX is from Thermothelomyces thermophilus (Tt).

The method of clause 98, wherein MIOX is TtMIOX according to SEQ ID NO: 173.

The method of clause 99, wherein TtMIOX is encoded by the nucleotide sequence SEQ ID NO: 174.

A yeast which is engineered to produce QA-C3-GlcA-Gal-Xyl according to the method of any one of clauses 93 to 100.

A method of producing UDP-Fucose (UDP-Fuc) in yeast, wherein the method comprises the step of overexpressing heterologous genes encoding the following enzymes:
a UDP-glucose-4,6-dehydratase (UG46DH) converting UDP-Glc into UDP-4-keto-6-deoxy-glucose and
a 4-keto-reductase converting UDP-4-keto-6-deoxy-glucose into UDP-D-Fuc.

The method of clause 102, wherein the UG46DH is from S. vaccaria (Sv).

The method of clause 103, wherein the UG46DH is SvUG46DH according to SEQ ID NO: 87.

The method of clause 104, wherein SvUG46DH is encoded by the nucleotide sequence SEQ ID NO: 88.

The method of any one of clauses 102 to 105, wherein the 4-keto-reductase is selected from Q. saponaria (Qs) and S. vaccaria (Sv).

The method of clause 106, wherein the 4-keto-reductase is selected from svNMD according to SEQ ID NO: 90 and QsFucSyn according to SEQ ID NO: 175.

The method of clause 107, wherein svNMD is encoded by the nucleotide sequence SEQ ID NO: 91 and QsFucSyn is encoded by the nucleotide sequence SEQ ID NO: 176.

A yeast which is engineered to produce UDP-Fucose according to the method of any one of clauses 102 to 108.

A method of producing a C28-glycosylated QA derivative in yeast, wherein the derivative is QA-C3-GlcA-Gal-Rha-C28-Fuc, or QA-C3-GlcA-Gal-Xyl-C28-Fuc, the method comprises the step of overexpressing, in a yeast engineered to produce QA-C3-GlcA-Gal-Rha, or QA-C3-GlcA-Gal-Xyl, and UDP-Fucose, a heterologous gene encoding the following enzyme:
a UDP-Fucose transferase (FucT) transferring UDP-Fuc and attaching a Fuc residue at the C28 position of QA to form QA-C3-GlcA-Gal-Rha-C28-Fuc, or QA-C3-GlcA-Gal-Xyl-C28-Fuc.

The method of clause 110, wherein the FucT is selected from Q. Saponaria (Qs) and S. vaccaria (Sv).

The method of clause 111, wherein the FucT is selected from QsFucT according to SEQ ID NO: 93 and SvFucT according to SEQ ID NO: 96.

The method of clause 112, wherein QsFucT is encoded by the nucleotide sequence SEQ ID NO: 94 and SvFucT is encoded by the nucleotide sequence SEQ ID NO: 97.

The method of any one of clauses 110 to 113, wherein the yeast engineered to produce QA-C3-GlcA-Gal-Rha is according to clause 87 and the yeast engineered to produce UDP-Fuc is according to clause 109.

The method of any one of clauses 110 to 113 wherein the yeast engineered to produce QA-C3-GlcA-Gal-Xyl is according to clause 101 and the yeast engineered to produce UDP-Fuc is according to clause 109.

A yeast which is engineered to produce QA-C3-GlcA-Gal-Rha-C28-Fuc according to the method of any one of clauses 110 to 114.

A yeast which is engineered to produce QA-C3-GlcA-Gal-Xyl-C28-Fuc according to the method of any one of clauses 110 to 113 and clause 115.

The method of any one of clauses 110 to 115, wherein the derivative is QA-C3-GlcA-Gal-Rha-C28-Fuc-Rha, or QA-C3-GlcA-Gal-Xyl-C28-Fuc-Rha, the overexpressing further comprises overexpressing a heterologous gene encoding the following enzyme:
a UDP-Rhamnose transferase (RhaT) transferring UDP-Rha and attaching a Rha residue to QA-C3-GlcA-Gal-Rha-C28-Fuc, or QA-C3-GlcA-Gal-Xyl-C28-Fuc, to form QA-C3-GlcA-Gal-Rha-C28-Fuc-Rha or QA-C3-GlcA-Gal-Xyl-C28-Fuc-Rha.

The method of clause 118, wherein the RhaT is from Q. saponaria.

The method of clause 119, wherein the RhaT is QsRhaT according to SEQ ID NO: 119.

The method of clause 120, wherein QsRhaT is encoded by the nucleotide sequence SEQ ID NO: 120.

A yeast which is engineered to produce QA-C3-GlcA-Gal-Rha-C28-Fuc-Rha or QA-C3-GlcA-Gal-Xyl-C28-Fuc-Rha according to the method of any one of clauses 118 to 121.

The method of any one of clauses 118 to 121, wherein the derivative is QA-C3-GlcA-Gal-Rha-C28-Fuc-Rha-Xyl, or QA-C3-GlcA-Gal-Xyl-C28-Fuc-Rha-Xyl, the overexpressing further comprises overexpressing heterologous genes encoding the following enzyme:
a UDP-Xylose transferase (XylT) transferring UDP-Xyl and attaching a Xyl residue to QA-C3-GlcA-Gal-Rha-C28-Fuc-Rha or QA-C3-GlcA-Gal-Xyl-C28-Fuc-Rha to form GlcA-Gal-Rha-C28-Fuc-Rha-Xyl and QA-C3-GlcA-Gal-Xyl-C28-Fuc-Rha-Xyl, respectively.

The method of clause 123, wherein the XylT is selected from Q. Saponaria (Qs) and S. vaccaria (Sv).

The method of clause 124, wherein the XylT is QsC28XylT3 according to SEQ ID NO: 125.

The method of clause 125, wherein QsC28XylT3 is encoded by the nucleotide sequence SEQ ID NO: 126.

A yeast which is engineered to produce QA-C3-GlcA-Gal-Rha-C28-Fuc-Rha-Xyl or QA-C3-GlcA-Gal-Xyl-C28-Fuc-Rha-Xyl according to the method of any of clauses 123 to 126.

The method of any one of clauses 123 to 126, wherein the derivative is QA-C3-GlcA-Gal-Rha-C28-Fuc-Rha-Xyl-Xyl, or QA-C3-GlcA-Gal-Xyl-C28-Fuc-Rha-Xyl-Xyl, the overexpressing further comprises overexpressing heterologous genes encoding the following enzymes:
a UDP-Xylose transferase (XylT) transferring UDP-Xyl and attaching a Xyl residue to QA-C3-GlcA-Gal-Rha-C28-Fuc-Rha-Xyl or QA-C3-GlcA-Gal-Xyl-C28-Fuc-Rha-Xyl to form QA-C3-GlcA-Gal-Rha-C28-Fuc-Rha-Xyl-Xyl and QA-C3-GlcA-Gal-Xyl-C28-Fuc-Rha-Xyl-Xyl, respectively.

The method of clause 128, wherein the XylT is selected from Q. Saponaria (Qs) and S. vaccaria (Sv).

The method of clause 129, wherein the XylT is QsC28XylT4 according to SEQ ID NO: 128.

The method of clause 130, wherein QsC28XylT4 is encoded by the nucleotide sequence SEQ ID NO: 129.

The method of clause 128 or clause 129, wherein QsC28XylT4 comprises an amino acid deletion at the N-terminus, ranging from 3 amino acids to 20 amino acids.

The method of clause 132, wherein the XylT is selected from QsC28XylT4-3aa according to SEQ ID NO: 131, QsC28XylT4-6aa according to SEQ ID NO: 134, QsC28XylT4-9aa according to SEQ ID NO: 137, and QsC28XylT4-12aa according to SEQ ID NO: 140.

The method of clause 133, wherein QsC28XylT4-3aa is encoded by the nucleotide sequence SEQ ID NO: 132, QsC28XylT4-6aa is encoded by the nucleotide sequence SEQ ID NO: 135, QsC28XylT4-9aa is encoded by the nucleotide sequence SEQ ID NO: 138, and QsC28XylT4-12aa is encoded by the nucleotide sequence SEQ ID NO: 141.

The method of clause 28 or clause 129, wherein a solubility tag is added at the N-terminus of XylT.

The method of clause 135, wherein the XylT is selected from SUMO-QsC28XylT4 according to SEQ ID NO: 143, TrxA-QsC28-XylT4 according to SEQ ID NO: 145, and MBP-QsC28XylT4 according to SEQ ID NO: 147.

The method of clause 136, wherein SUMO-QsC28XylT4 is encoded by the nucleotide sequence SEQ ID NO: 144, TrxA-QsC28-XylT4 is encoded by the nucleotide sequence SEQ ID NO: 146 and MBP-QsC28XylT4 is encoded by the nucleotide sequence SEQ ID NO: 148.

The method of clause 128 or clause 129, wherein the XylT is QsC28XylT3-3×GGGS-QsC28XylT4 according to SEQ ID NO: 149.

The method of clause 138, wherein QsC28XylT3-3×GGGS-QsC28XylT4 is encoded by the nucleotide sequence SEQ ID NO: 150.

A yeast which is engineered to produce QA-C3-GlcA-Gal-Rha-C28-Fuc-Rha-Xyl-Xyl or QA-C3-GlcA-Gal-Xyl-C28-Fuc-Rha-Xyl-Xyl according to the method of any of clauses 128 to 139.

The method of any one of clauses 123 to 126, wherein the derivative is QA-C3-GlcA-Gal-Rha-C28-Fuc-Rha-Xyl-Api or QA-C3-GlcA-Gal-Xyl-C28-Fuc-Rha-Xyl-Api, the overexpressing further comprises overexpressing heterologous genes encoding the following enzymes:
a UDP-Apiose synthase (AXS) converting UDP-GlcA into UDP-Api and
a UDP-Apiose transferase (ApiT) transferring UDP-Apiose and attaching an Apiose residue to QA-C3-GlcA-Gal-Rha-C28-Fuc-Rha-Xyl or QA-C3-GlcA-Gal-Xyl-C28-Fuc-Rha-Xyl to form QA-C3-GlcA-Gal-Rha-C28-Fuc-Rha-Xyl-Api and QA-C3-GlcA-Gal-Xyl-C28-Fuc-Rha-Xyl-Api, respectively.

The method of clause 141, wherein the AXS is QsAXS according to SEQ ID NO: 113.

The method of clause 142, wherein QsAXS is encoded by the nucleotide sequence SEQ ID NO: 114.

The method of any one of clauses 141 to 143, wherein the ApiT is selected from Q. saponaria (Qs) or S. vaccaria (Sv).

The method of clause 144, wherein the ApiT is QsC28ApiT4 according to SEQ ID NO: 151.

The method of clause 145, wherein QsC28ApiT4 is encoded by the nucleotide sequence SEQ ID NO: 152.

A yeast which is engineered to produce QA-C3-GlcA-Gal-Rha-C28-Fuc-Rha-Xyl-Api or QA-C3-GlcA-Gal-Xyl-C28-Fuc-Rha-Xyl-Api according to the method of any of clauses 141 to 146.

A method of producing (S)-2-methylbutyryl CoA (2 MB-CoA) in yeast, wherein the method comprises the step of overexpressing a heterologous gene encoding (i) a carboxyl coenzyme A (CoA) ligase (CCL) converting 2 MB acid into 2 MB-CoA, and 2 MB acid is supplemented exogenously.

The method of clause 148, wherein the CCL is QsCCL from Q. saponaria according to SEQ ID NO: 178.

The method of clause 149, wherein QsCCL is encoded by the nucleotide sequence SEQ ID NO: 179.

The method of any one of clauses 148 to 150 in yeast, wherein the overexpressing further comprises overexpressing heterologous genes encoding the following enzymes:
a phosphopantetheinyl (Ppant) transferase,
a megasynthase LovF-TE including an ACP domain, condensing two units of malonyl-CoA to 2 MB-ACP, cleaving 2 MB acid from the ACP domain which is converted into 2 MB-CoA by the CCL,
and no 2 MB acid is supplemented exogenously.

The method of clause 151, wherein the Ppant is from Aspergillus nidulans (An) and the megasynthase LovF-TE is from Aspergillus terreus (Ast).

The method of clause 152, wherein the Ppant is AnNpgA according to SEQ ID NO: 237 and the megasynthase LovF-TE is AstLovF-TE according to SEQ ID NO: 235.

The method of clause 153, wherein AnNgA is encoded by the nucleotide sequence SEQ ID NO: 238 and AstLovF-TE is encoded by the nucleotide sequence SEQ ID NO: 236.

A yeast engineered to produce 2 MB-CoA according to the method of any one of clauses 148 to 154.

A method of producing UDP-Arabinofuranose (UDP-Araf) in yeast, wherein the method comprises the step of overexpressing, in a yeast engineered to produce UDP-Xyl, heterologous genes encoding the following enzymes:
a UDP-Xyl epimerase (UXE) converting UDP-Xyl into UDP-Arabinopyranose (UDP-Arap), and
a UDP-Arabinose mutases (UAM) converting UDP-Arap into UDP-Arabinofuranose (UDP-Araf).

The method of clause 156, wherein the UXE and the UAM are independently selected from A. thaliana (At) and H. vulgare (Hv).

The method of clause 157, wherein the UXE is selected from AtUXE according to SEQ ID NO: 199, AtUXE2 according to SEQ ID NO: 202, HvUXE-1 according to SEQ ID NO: 240, HvUXE-2 according to SEQ ID NO: 242 and AtUGE3 according to SEQ ID NO: 205 and the UAM is selected from AtUAM1 according to SEQ ID NO: 208 and HvUAM according to SEQ ID NO: 211.

The method of clause 158, wherein AtUXE is encoded by the nucleotide sequence SEQ ID NO: 200, AtUXE2 is encoded by the nucleotide sequence SEQ ID NO: 203, HvUXE-1 is encoded by the nucleotide sequence SEQ ID NO: 241, HvUXE-2 is encoded by the nucleotide sequence SEQ ID NO: 243, AtUAM1 is encoded by the nucleotide sequence SEQ ID NO: 209, HvUAM is encoded by the nucleotide sequence SEQ ID NO: 212, and AtUGE3 is encoded by the nucleotide sequence SEQ ID NO: 206.

The method of any one of clauses 156 to 159, wherein the yeast engineered to produce UDP-Xyl is according to clause 62.

A yeast which is engineered to produce UDP-Araf according to the method of any of clauses 156 to 160.

A method of producing UDP-Araf in yeast, wherein the method comprises the step of overexpressing heterologous genes encoding the following enzymes:
an arabinokinase (AraK) and
a UDP-sugar pyrophosphorylase (USP),
and arabinose is supplemented exogenously.

The method of clause 162, wherein the AraK and the USP are independently selected from A. thaliana (At) and Leptospira interrogans (Lei).

The method of clause 163, wherein the AraK is selected from AtAraK according to SEQ ID NO: 214 and LeiAraK according to SEQ ID NO: 217 and the USP is selected from AtUSP according to SEQ ID NO: 223 and LeiUSP according to SEQ ID NO: 226.

The method of clause 164, wherein the AtAraK is encoded by the nucleotide sequence SEQ ID NO: 215, LeiAraK is encoded by the nucleotide sequence SEQ ID NO: 218, AtUSP is encoded by the nucleotide sequence SEQ ID NO: 224 and LeiUSP is encoded by the nucleotide sequence SEQ ID NO: 227.

The method of any one of clauses 162 to 165, wherein the overexpressing further comprises overexpressing a heterologous gene encoding (iii) an arabinose transporter (AraT).

The method of clause 166, wherein the AraT is PrAraT from Penicillium rubens Wisconsin according to SEQ ID NO: 220.

The method of clause 167, wherein PrAraT is encoded by the nucleotide sequence SEQ ID NO: 221.

A yeast which is engineered to produce UDP-Araf according to the method of any one of clauses 162 to 168.

A method of producing an acylated and glycosylated QA derivative in yeast, wherein the derivative is QA-C3-GGR-C28-FRX-C9, QA-C3-GGX-C28-FRX-C9, QA-C3-GGR-C28-FRXX-C9, QA-C3-GGX-C28-FRXX-C9, QA-C3-GGR-C28-FRXA-C9 or QA-C3-GGX-C28-FRXA-C9, and the method comprises the step of overexpressing, in a yeast engineered to produce QA-C3-GGR-C28-FRX, QA-C3-GGX-C28-FRX, QA-C3-GGR-C28-FRXX, QA-C3-GGX-C28-FRXX, QA-C3-GGR-C28-FRXA, or QA-C3-GGX-C28-FRXA, heterologous genes encoding the following enzymes:
a carboxyl coenzyme A ligase (CCL) converting 2 MB acid into 2 MB-CoA,
a chalcone-synthase-like type III PKS (Polyketide synthase) condensing malonyl-CoA with 2 MB-CoA to form C9-Keto-CoA,
a keto-reductase (KR) converting C9-Keto-CoA into C9-CoA, and
an acyltransferase transferring and attaching a first C9-CoA unit to QA-C3-GGR-C28-FRX, QA-C3-GGX-C28-FRX, QA-C3-GGR-C28-FRXX, QA-C3-GGX-C28-FRXX, QA-C3-GGR-C28-FRXA, or QA-C3-GGX-C28-FRXA to form QA-C3-GGR-C28-FRX-C9, QA-C3-GGX-C28-FRX-C9, QA-C3-GGR-C28-FRXX-C9, QA-C3-GGX-C28-FRXX-C9, QA-C3-GGR-C28-FRXA-C9 or QA-C3-GGX-C28-FRXA-C9.
wherein 2 MB acid is supplemented exogenously.

The method of clause 170, wherein the CCL, the chalcone-synthase-like type III PKS, the KR and the acyltransferase are from Q. saponaria.

The method of clause 171, wherein the CCL is QsCCL according to SEQ ID NO: 178, the chalcone-synthase-like type III PKS is QsChSD according to SEQ ID NO: 181, QsChSE according to SEQ ID NO: 184, or both QsChSD according to SEQ ID NO:181 and QsChSE according to SEQ ID NO: 184, the keto-reductase is QsKR11 according to SEQ ID NO: 187, QsKR23 according to SEQ ID NO: 190, or both QsKR11 according to SEQ ID NO: 187 and QsKR23 according to SEQ ID NO: 190, and the acyltransferase is QsDMOT9 according to SEQ ID NO: 193.

The method of clause 171, wherein the chalcone-synthase-like type III PKS are both QsChSD according to SEQ ID NO: 181 and QsChSE according to SEQ ID NO: 184.

The method of clause 170 wherein the KR are both QsKR11 according to SEQ ID NO: 187 and QsKR23 according to SEQ ID NO: 190.

The method of any one of clauses 172 to 174, wherein the CCL is QsCCL according to SEQ ID NO: 178, the chalcone-synthase-like type III PKS are QsChSD according to SEQ ID NO: 181 and QsChSE according to SEQ ID NO: 184, the KR are QsKR11 according to SEQ ID NO: 187 and QsKR23 according to SEQ ID NO: 190 and the acyltransferase is QsDMOT9 according to SEQ ID NO: 193.

The method of clause 175, wherein QsCCL is encoded by SEQ ID NO: 179, QsChSD is encoded by the nucleotide sequence SEQ ID NO: 182, QsChSE is encoded by the nucleotide sequence SEQ ID NO: 185, QsKR11 is encoded by the nucleotide sequence SEQ ID NO: 188, QsKR23 is encoded by the nucleotide sequence SEQ ID NO: 191 and QsDMOT9 is encoded by the nucleotide sequence SEQ ID NO: 194.

The method of any one of clauses 170 to 176, wherein the yeast engineered to produce QA-C3-GGR-C28-FRX and QA-C3-GGX-C28-FRX is according to clause 127, the yeast engineered to produce QA-C3-GGR-C28-FRXX and QA-C3-GGX-C28-FRXX is according to clause 140 and the yeast engineered to produce QA-C3-GGR-C28-FRXA and QA-C3-GGX-C28-FRXA is according to clause 147.

A yeast which is engineered to produce QA-C3-GGR-C28-FRX-C9, QA-C3-GGX-C28-FRX-C9, QA-C3-GGR-C28-FRXX-C9, QA-C3-GGX-C28-FRXX-C9, QA-C3-GGR-C28-FRXA-C9, or QA-C3-GGX-C28-FRXA-C9 according to the method of any one of clauses 170 to 177.

The method of any one of clauses 170 to 178, wherein the derivative is QA-C3-GGR-C28-FRX-C18, QA-C3-GGX-C28-FRX-C18, QA-C3-GGR-C28-FRXX-C18, QA-C3-GGX-C28-FRXX-C18, QA-C3-GGR-C28-FRXA-C18 or QA-C3-GGX-C28-FRXA-C18, and the overexpressing further comprises overexpressing a heterologous gene encoding the following enzyme:
an acyltransferase QsDMOT4 according to SEQ ID NO: 196 attaching a second C9-CoA unit to C3-GGR-C28-FRX-C9, QA-C3-GGX-C28-FRX-C9, QA-C3-GGR-C28-FRXX-C9, QA-C3-GGX-C28-FRXX-C9, QA-C3-GGR-C28-FRXA-C9, or QA-C3-GGX-C28-FRXA-C9 to form C3-GGR-C28-FRX-C18, QA-C3-GGX-C28-FRX-C18, QA-C3-GGR-C28-FRXX-C18, QA-C3-GGX-C28-FRXX-C18, QA-C3-GGR-C28-FRXA-C18 or QA-C3-GGX-C28-FRXA-C18.

The method of clause 179, wherein QsDMOT4 is encoded by the nucleotide sequence SEQ ID NO: 197.

A yeast which is engineered to produce QA-C3-GGX-C28-FRX-C18, QA-C3-GGR-C28-FRX-C18, QA-C3-GGX-C28-FRXX-C18, QA-C3-GGR-C28-FRXX-C18, QA-C3-GGX-C28-FRXA-C18, or QA-C3-GGR-C28-FRXA-C18 according to the method of clause 179 or clause 180.

The method of any one of clauses 179 or 180, wherein the derivative is QA-C3-GGR-C28-FRX-C18-Araf, QA-C3-GGX-C28-FRX-C18-Araf, QA-C3-GGR-C28-FRXX-C18-Araf, QA-C3-GGX-C28-FRXX-C18-Araf, QA-C3-GGR-C28-FRXA-C18-Araf, or QA-C3-GGX-C28-FRXA-C18-Araf, the yeast is further engineered to produce UDP-Araf, and the overexpressing further comprises overexpressing a heterologous gene encoding the following enzyme:
an arabinotransferase (ArafT) transferring UDP-Araf and attaching an Araf residue to QA-C3-GGR-C28-FRX-C18, QA-C3-GGX-C28-FRX-C18, QA-C3-GGR-C28-FRXX-C18, QA-C3-GGX-C28-FRXX-C18, QA-C3-GGR-C28-FRXA-C18, or QA-C3-GGX-C28-FRXA-C18- to form QA-C3-GGR-C28-FRX-C18-Araf, QA-C3-GGX-C28-FRX-C18-Araf, QA-C3-GGR-C28-FRXX-C18-Araf, QA-C3-GGX-C28-FRXX-C18-Araf, QA-C3-GGR-C28-FRXA-C18-Ara for QA-C3-GGX-C28-FRXA-C18-Araf.

The method of clause 182, wherein the ArafT is from Q. saponaria (Qs).

The method of clause 182 or clause 183, wherein the ArafT is selected from QsArafT according to SEQ ID NO: 229 and QsArafT2 according to SEQ ID NO: 232.

The method of clause 184, wherein QsArafT is encoded by the nucleotide sequence SEQ ID NO: 230, and QsArafT2 is encoded by the nucleotide sequence SEQ ID NO: 233.

The method of clause 184, wherein the ArafT is QsArafT2 according to SEQ ID NO: 232.

The method of clause 186, wherein QsArafT2 is encoded by the nucleotide sequence SEQ ID NO: 233.

The method of any one of clauses 182 to 187, wherein the yeast engineered to produce UDP-Araf is according to clause 161 or clause 169.

A yeast which is engineered to produce QA-C3-GGR-C28-FRX-C18-Araf, QA-C3-GGX-C28-FRX-C18-Araf, QA-C3-GGR-C28-FRXX-C18-Araf, QA-C3-GGX-C28-FRXX-C18-Araf, QA-C3-GGR-C28-FRXA-C18-Araf, or QA-C3-GGX-C28-FRXA-C18-Araf according to the method of any one of clauses 182 to 188.

A method of producing QA-C3-GGX-C28-FRX-C18-Xyl, QA-C3-GGR-C28-FRX-C18-Xyl, QA-C3-GGX-C28-FRXX-C18-Xyl, QA-C3-GGR-C28-FRXX-C18-Xyl, QA-C3-GGX-C28-FRX-C18-Xyl, QA-C3-GGX-C28-FRXA-C18-Xyl or QA-C3-GGR-C28-FRX-C18-Xyl in a yeast, wherein the method comprises the step of overexpressing, in a yeast engineered to produce QA-C3-GGX-C28-FRX-C18, QA-C3-GGR-C28-FRX-C18, QA-C3-GGX-C28-FRXX-C18, QA-C3-GRX-C28-FRXX-C18, QA-C3-GGX-C28-FRX-C18, QA-C3-GGX-C28-FRXA-C18 or QA-C3-GGR-C28-FRX-C18, a heterologous gene encoding an arabinotransferase (ArafT) transferring UDP-Xyl and attaching a Xyl residue to QA-C3-GGX-C28-FRX-C18, QA-C3-GGR-C28-FRX-C18, QA-C3-GGX-C28-FRXX-C18, QA-C3-GRX-C28-FRXX-C18, QA-C3-GGX-C28-FRX-C18, QA-C3-GGX-C28-FRXA-C18 and QA-C3-GGR-C28-FRX-C18 to form QA-C3-GGX-C28-FRX-C18-Xyl, QA-C3-GGR-C28-FRX-C18-Xyl, QA-C3-GGX-C28-FRXX-C18-Xyl, QA-C3-GRX-C28-FRXX-C18-Xyl, QA-C3-GGX-C28-FRX-C18-Xyl, QA-C3-GGX-C28-FRXA-C18-Xyl or QA-C3-GGR-C28-FRX-C18-Xyl.

The method of clause 190, wherein the ArafT is QsArafT is according to SEQ ID NO: 229.

The method of clause 191, wherein QsArafT is encoded by the nucleotide sequence SEQ ID NO: 230.

The method of any one of clauses 190 to 192, wherein the yeast engineered to produce QA-C3-GGX-C28-FRX-C18, QA-C3-GGR-C28-FRX-C18, QA-C3-GGX-C28-FRXX-C18, QA-C3-GRX-C28-FRXX-C18, QA-C3-GGX-C28-FRX-C18, QA-C3-GGX-C28-FRXA-C18 and QA-C3-GGR-C28-FRX-C18 is according to clause 181.

A yeast engineered to produce QA-C3-GGX-C28-FRX-C18-Xyl, QA-C3-GGR-C28-FRX-C18-Xyl, QA-C3-GGX-C28-FRXX-C18-Xyl, QA-C3-GRX-C28-FRXX-C18-Xyl, QA-C3-GGX-C28-FRX-C18-Xyl, QA-C3-GGX-C28-FRXA-C18-Xyl or QA-C3-GGR-C28-FRX-C18-Xyl according to the method of any one of clauses 190 to 193.

The method of any one of clauses 170 to 177, 179 to 180, 182 to 188 and 190 to 193, wherein the overexpressing further comprises the overexpressing of heterologous genes encoding the following enzymes:
a phosphopantetheinyl (Ppant) transferase,
a megasynthase LovF-TE including an ACP domain, condensing two units of malonyl-CoA to 2 MB-ACP, cleaving 2 MB acid from the ACP domain which is converted into 2 MB-CoA by the CoA ligase (CCL),
and no 2 MB acid is supplemented exogenously.

The method of clause 195, wherein the Ppant is from Aspergillus nidulans (An) and the megasynthase LovF-TE is from Aspergillus terreus (Ast).

The method of clause 196, wherein the Ppant is AnNpgA according to SEQ ID NO: 237 and the megasynthase LovF-TE is AstLovF-TE according to SEQ ID NO: 235.

The method of clause 197, wherein AnNgA is encoded by the nucleotide sequence SEQ ID NO: 238 and AstLovF-TE is encoded by the nucleotide sequence SEQ ID NO: 236.

A yeast which is engineered to produce QA-C3-GGR-C28-FRX-C18-Araf, QA-C3-GGX-C28-FRX-C18-Araf, QA-C3-GGR-C28-FRXX-C18-Araf, QA-C3-GGX-C28-FRXX-C18-Araf, QA-C3-GGR-C28-FRXA-C18-Araf, or QA-C3-GGX-C28-FRXA-C18-Araf according to the method of any one of clauses 195 to 198.

A method of producing QA-C3-GGX-C28-FRXX-C18-Araf (QS-21-Xyl) in yeast, wherein the method comprises the step of overexpressing heterologous genes encoding GvBAS according to SEQ ID NO: 10, QsC28C16 according to SEQ ID NO: 23, QsC23 according to SEQ ID NO: 29, QsC28 according to SEQ ID NO: 41, AtATR1 according to SEQ ID NO: 49, Qsb5 according to SEQ ID NO: 55, SvMSBP1 according to SEQ ID NO: 67, AtUGD_{A101L} according to SEQ ID NO: 108, QsCslG2 according to SEQ ID NO: 78, QsGalT according to SEQ ID NO: 116, AtUXS according to SEQ ID NO: 105, QsC3XylT according to SEQ ID NO: 122, SvNMD according to SEQ ID NO: 90, SvUG46DH according to SEQ ID NO: 87, QsFuct according to SEQ ID NO: 93, AtRHM2 according to SEQ ID NO: 102, QsRhaT according to SEQ ID NO: 119, QsC28XylT3 according to SEQ ID NO: 125, QsC28XylT4 according to SEQ ID NO: 128, QsChSD according to SEQ ID NO: 181, QsChSE according to SEQ ID NO: 184, QsKR11 according to SEQ ID NO: 187, QsKR23 according to SEQ ID NO: 190, QsDMOT9 according to SEQ ID NO: 193, QsDMOT4 according to SEQ ID NO: 196, AtUXE according to SEQ ID NO: 199, AtUAM1 according to SEQ ID NO: 208, QsArafT2 according to SEQ ID NO: 232, AnNpgA according to SEQ ID NO: 237, QsCCL according to SEQ ID NO: 178 and AstLovF-TE according to SEQ ID NO: 235.

The method of clause 200, wherein GvBAS is encoded by the nucleotide sequence SEQ ID NO: 11, QsC28C16 is encoded by the nucleotide sequence SEQ ID NO: 24, QsC23 is encoded by the nucleotide sequence SEQ ID NO: 30, QsC28 is encoded by the nucleotide sequence SEQ ID NO: 42, AtATR1 is encoded by the nucleotide sequence SEQ ID NO: 50, Qsb5 is encoded by the nucleotide sequence SEQ ID NO: 56, SvMSBP1 is encoded by the nucleotide sequence SEQ ID NO: 68, AtUGD_{A101L} is encoded by the nucleotide sequence SEQ ID NO: 109, QsCslG2 is encoded by the nucleotide sequence SEQ ID NO: 82, QsGalT is encoded by the nucleotide sequence SEQ ID NO: 117, AtUXS is encoded by the nucleotide sequence SEQ ID NO: 106, QsC3XylT is encoded by the nucleotide sequence SEQ ID NO: 123, SvNMD is encoded by the nucleotide sequence SEQ ID NO: 91, SvUG46DH is encoded by the nucleotide sequence SEQ ID NO: 88, QsFucT is encoded by the nucleotide sequence SEQ ID NO: 94, AtRHM2 is encoded by the nucleotide sequence SEQ ID NO: 103, QsRhaT is encoded by the nucleotide sequence SEQ ID NO: 220, QsC28XylT3 is encoded by the nucleotide sequence SEQ ID NO: 126, QsC28XylT4 is encoded by the nucleotide sequence SEQ ID NO: 129, QsChSD is encoded by the nucleotide sequence SEQ ID NO: 182, QsChSE is encoded by the nucleotide sequence SEQ ID NO: 185, QsKR11 is encoded by the nucleotide sequence SEQ ID NO: 188, QsKR23 is encoded by the nucleotide sequence SEQ ID NO: 191, QsDMOT9 is encoded by the nucleotide sequence SEQ ID NO: 194, QsDMOT4 is encoded by the nucleotide sequence SEQ ID NO: 197, AtUXE is encoded by the nucleotide sequence SEQ ID NO: 200, AtUAM1 is encoded by the nucleotide sequence SEQ ID NO: 209, QsArafT2 is encoded by the nucleotide sequence SEQ ID NO: 233, AnNpgA is encoded by the nucleotide sequence SEQ ID NO: 238, QsCCL is encoded by the nucleotide sequence SEQ ID NO: 179 and AstLovF-TE is encoded by the nucleotide sequence SEQ ID NO: 236.

A yeast which is engineered to produce QS-21-Xyl according to the method of clause 201 or clause 202.

A method of producing QA-C3-GGX-C28-FRXA-C18-Araf (QS-21-Api) in yeast, wherein the method comprises the step of overexpressing heterologous genes encoding GvBAS according to SEQ ID NO: 10, QsC28C16 according to SEQ ID NO: 23, QsC23 according to SEQ ID NO: 29, QsC28 according to SEQ ID NO: 41, AtATR1 according to SEQ ID NO: 49, Qsb5 according to SEQ ID NO: 55, SvMSBP1 according to SEQ ID NO: 67, AtUGD_{A101L} according to SEQ ID NO: 108, QsCslG2 according to SEQ ID NO: 81, QsGalT according to SEQ ID NO: 116, AtUXS according to SEQ ID NO: 105, QsC3XylT according to SEQ ID NO: 122, SvNMD according to SEQ ID NO: 90, SvUG46DH according to SEQ ID NO: 87, QsFucT according to SEQ ID NO: 93, AtRHM2 according to SEQ ID NO: 102, QsRhaT according to SEQ ID NO: 119, QsC28XylT3 according to SEQ ID NO: 125, QsC28ApiT4 according to SEQ ID NO: 151, QsChSD according to SEQ ID NO: 181, QsChSE according to SEQ ID NO: 184, QsKR11 according to SEQ ID NO: 187, QsKR23 according to SEQ ID NO: 190, QsDMOT9 according to SEQ ID NO: 193, QsDMOT4 according to SEQ ID NO: 196, AtUXE according to SEQ ID NO: 199, AtUAM1 according to SEQ ID NO: 208, QsArafT2 according to SEQ ID NO: 232, AnNpgA according to SEQ ID NO: 237, QsCCL according to SEQ ID NO: 178 and AstLovF-TE according to SEQ ID NO: 235.

The method of clause 203, wherein GvBAS is encoded by the nucleotide sequence SEQ ID NO: 11, QsC28C16 is encoded by the nucleotide sequence SEQ ID NO: 24, QsC23 is encoded by the nucleotide sequence SEQ ID NO: 30, QsC28 is encoded by the nucleotide sequence SEQ ID NO: 42, AtATR1 is encoded by the nucleotide sequence SEQ ID NO: 50, Qsb5 is encoded by the nucleotide sequence SEQ ID NO: 56, SvMSBP1 is encoded by the nucleotide sequence SEQ ID NO: 68, AtUGD_{A101L} is encoded by the nucleotide sequence SEQ ID NO: 109, QsCslG2 is encoded by the nucleotide sequence SEQ ID NO: 82, QsGalT is encoded by the nucleotide sequence SEQ ID NO: 117, AtUXS is encoded by the nucleotide sequence SEQ ID NO: 106, QsC3XylT is encoded by the nucleotide sequence SEQ ID NO: 123, SvNMD is encoded by the nucleotide sequence SEQ ID NO: 91, SvUG46DH is encoded by the nucleotide sequence SEQ ID NO: 88, QsFucT is encoded by the nucleotide sequence SEQ ID NO: 94, AtRHM2 is encoded by the nucleotide sequence SEQ ID NO: 103, QsRhaT is encoded by the nucleotide sequence SEQ ID NO: 120, QsC28XylT3 is encoded by the nucleotide sequence SEQ ID NO: 126, QsC28ApiT4 is encoded by the nucleotide sequence SEQ ID NO: 152, QsChSD is encoded by the nucleotide sequence SEQ ID NO: 182, QsChSE is encoded by the nucleotide sequence SEQ ID NO: 185, QsKR11 is encoded by the nucleotide sequence SEQ ID NO: 188, QsKR23 is encoded by the nucleotide sequence SEQ ID NO: 191, QsDMOT9 is encoded by the nucleotide sequence SEQ ID NO: 194, QsDMOT4 is encoded by the nucleotide sequence SEQ ID NO: 197, AtUXE is encoded by the nucleotide sequence SEQ ID NO: 200, AtUAM1 is encoded by the nucleotide sequence SEQ ID NO: 209, QsArafT2 is encoded by the nucleotide sequence SEQ ID NO: 233, AnNpgA is encoded by the nucleotide sequence SEQ ID NO: 238, QsCCL is encoded by the nucleotide sequence SEQ ID NO: 179 and AstLovF-TE is encoded by the nucleotide sequence SEQ ID NO: 236.

A yeast which is engineered to produce QS-21-Api according to the method of clause 204 or clause 205.

The method of any one of clauses 1 to 38, 42 to 45, 47 to 50, 52 to 61, 63 to 72, 74 to 80, 82 to 86, 88 to 91, 93 to 100, 102 to 108, 110 to 115, 118 to 121, 123 to 126, 128 to 139, 141 to 146, 148 to 154, 156 to 160, 162 to 168, 170 to 177, 179, 180, 182 to 188, 190 to 193, 195 to 198, 200, 201, 203 and 204, or the yeast of any one of clauses 39, 40, 46, 51, 62, 73, 81, 87, 92, 101, 109, 116, 117, 122, 127, 140, 147, 155, 161, 169, 178, 181, 189, 194, 199, 202 and 205, wherein GvBAS (when present) is according to an amino acid sequence at least 70%, 80%, 90%, 95%, 98%, or 99% identical to SEQ ID NO: 10, QsC28C16 (when present) is according to an amino acid sequence at least 70%, 80%, 90%, 95%, 98%, or 99% identical to according to SEQ ID NO: 23, QsC23 (when present) is according to an amino acid sequence at least 70%, 80%, 90%, 95%, 98%, or 99% identical to according to SEQ ID NO: 29, QsC28 (when present) is according to an amino acid sequence at least 70%, 80%, 90%, 95%, 98%, or 99% identical to SEQ ID NO: 41, AtATR1 (when present) is according to an amino acid sequence at least 70%, 80%, 90%, 95%, 98%, or 99% identical to SEQ ID NO: 49, Qsb5 (when present) is according to an amino acid sequence at least 70%, 80%, 90%, 95%, 98%, or 99% identical to SEQ ID NO: 55, SvMSBP1 (when present) is according to an amino acid sequence at least 70%, 80%, 90%, 95%, 98%, or 99% identical to SEQ ID NO: 67, AtUGD_{A101L} (when present) is according to an amino acid sequence at least 70%, 80%, 90%, 95%, 98%, or 99% identical to SEQ ID NO: 108, QsCslG2 (when present) is according to an amino acid sequence at least 70%, 80%, 90%, 95%, 98%, or 99% identical to SEQ ID NO: 81, QsGalT (when present) is according to an amino acid sequence at least 70%, 80%, 90%, 95%, 98%, or 99% identical to SEQ ID NO: 116, AtUXS (when present) is according to an amino acid sequence at least 70%, 80%, 90%, 95%, 98%, or 99% identical to SEQ ID NO: 105, QsC3XylT (when present) is according to an amino acid sequence at least 70%, 80%, 90%, 95%, 98%, or 99% identical to SEQ ID NO: 122, SvNMD (when present) is according to an amino acid sequence at least 70%, 80%, 90%, 95%, 98%, or 99% identical to SEQ ID NO: 90, SvUG46DH (when present) is according to an amino acid sequence at least 70%, 80%, 90%, 95%, 98%, or 99% identical to SEQ ID NO: 87, QsFucT (when present) is according to an amino acid sequence at least 70%, 80%, 90%, 95%, 98%, or 99% identical to SEQ ID NO: 93, AtRHM2 (when present) is according to an amino acid sequence at least 70%, 80%, 90%, 95%, 98%, or 99% identical to SEQ ID NO: 102, QsC28XylT3 (when present) is according to an amino acid sequence at least 70%, 80%, 90%, 95%, 98%, or 99% identical to SEQ ID NO: 125, QsC28XylT4 (when present) is according to an amino acid sequence at least 70%, 80%, 90%, 95%, 98%, or 99% identical to SEQ ID NO: 128, QsC28ApiT4 (when present) is according to an amino acid sequence at least 70%, 80%, 90%, 95%, 98%, or 99% identical to SEQ ID NO: 151, QsChSD (when present) is according to an amino acid sequence at least 70%, 80%, 90%, 95%, 98%, or 99% identical to SEQ ID NO: 181, QsChSE according to SEQ ID NO: 184, QsKR11 (when present) is according to an amino acid sequence at least 70%, 80%, 90%, 95%, 98%, or 99% identical to SEQ ID NO: 187, QsKR23 (when present) is according to an amino acid sequence at least 70%, 80%, 90%, 95%, 98%, or 99% identical to SEQ ID NO: 190, QsDMOT9 (when present) is according to an amino acid sequence at least 70%, 80%, 90%, 95%, 98%, or 99% identical to SEQ ID NO: 193, QsDMOT4 (when present) is according to an amino acid sequence at least 70%, 80%, 90%, 95%, 98%, or 99% identical to SEQ ID NO: 196, AtUXE (when present) is according to an amino acid sequence at least 70%, 80%, 90%, 95%, 98%, or 99% identical to SEQ ID NO: 199, AtUAM1 (when present) is according to an amino acid sequence at least 70%, 80%, 90%, 95%, 98%, or 99% identical to SEQ ID NO: 208, QsArafT2 (when present) is according to an amino acid sequence at least 70%, 80%, 90%, 95%, 98%, or 99% identical to SEQ ID NO: 232, AnNpgA (when present) is according to an amino acid sequence at least 70%, 80%, 90%, 95%, 98%, or 99% identical to SEQ ID NO: 237, QsCCL (when present) is according to an amino acid sequence at least 70%, 80%, 90%, 95%, 98%, or 99% identical to SEQ ID NO: 178 and AstLovF-TE (when present) is according to an amino acid sequence at least 70%, 80%, 90%, 95%, 98%, or 99% identical to SEQ ID NO: 235.

The method of any one of clauses 1 to 38, 42 to 45, 47 to 50, 52 to 61, 63 to 72, 74 to 80, 82 to 86, 88 to 91, 93 to 100, 102 to 108, 110 to 115, 118 to 121, 123 to 126, 128 to 139, 141 to 146, 148 to 154, 156 to 160, 162 to 168, 170 to 177, 179, 180, 182 to 188, 190 to 193, 195 to 198, 200, 201, 203, 204 and 206, or the yeast of any one of clauses 39, 40, 46, 51, 62, 73, 81, 87, 92, 101, 109, 116, 117, 122, 127, 140, 147, 155, 161, 169, 178, 181, 189, 194, 199, 202, 205 and 206, wherein the heterologous genes are integrated into the genome of the yeast.

The method, or yeast, of clause 207, wherein one or more copies of one or more of the heterologous genes are integrated.

The method, or yeast, of clause 208, wherein the one or more copies ranges from 2 to 5.

The method, or yeast, of clause 208 or clause 209, wherein at least 2 copies of the genes encoding the C16 oxidase, the C23 oxidase and the C28 oxidase are integrated.

The method, or yeast, of any one of clauses 208 to 210, wherein at least 3 copies of the gene encoding the UXS (when present) are integrated.

The method, or yeast, of clause 208 to 211, wherein the nucleotide sequence of the heterologous genes is codon-optimized.

The method, or yeast, of clause 212, wherein GvBAS (when present) is encoded by the nucleotide sequence SEQ ID NO: 12, QsC28C16 (when present) is encoded by the nucleotide sequence SEQ ID NO: 25, QsC23 (when present) is encoded by the nucleotide sequence SEQ ID NO: 31, QsC28 (when present) is encoded by the nucleotide sequence SEQ ID NO: 43, AtATR1 (when present) is encoded by the nucleotide sequence SEQ ID NO: 51, Qsb5 (when present) is encoded by the nucleotide sequence SEQ ID NO: 57, SvMSBP1 is encoded by the nucleotide sequence SEQ ID NO: 69, AtUGD_{A101L} (when present) is encoded by the nucleotide sequence SEQ ID NO: 109, QsCslG2 (when present) is encoded by the nucleotide sequence SEQ ID NO: 83, QsGalT (when present) is encoded by the nucleotide sequence SEQ ID NO: 118, AtUXS (when present) is encoded by the nucleotide sequence SEQ ID NO: 107, QsC3XylT (when present) is encoded by the nucleotide sequence SEQ ID NO: 124, SvNMD (when present) is encoded by the nucleotide sequence SEQ ID NO: 92, SvUG46DH (when present) is encoded by the nucleotide sequence SEQ ID NO: 89, QsFucT (when present) is encoded by the nucleotide sequence SEQ ID NO: 95, AtRHM2 (when present) is encoded by the nucleotide sequence SEQ ID NO: 104, QsRhaT (when present) is encoded by the nucleotide sequence SEQ ID NO: 121, QsC28XylT3 (when present) is encoded by the nucleotide sequence SEQ ID NO: 127, QsC28XylT4 (when present) is encoded by the nucleotide sequence SEQ ID NO: 130, QsC28ApiT4 (when present) encoded by the nucleotide sequence SEQ ID NO: 153 QsChSD (when present) is encoded by the nucleotide sequence SEQ ID NO: 183, QsChSE (when present) is encoded by the nucleotide sequence SEQ ID NO: 186, QsKR11 (when present) is encoded by the nucleotide sequence SEQ ID NO: 189, QsKR23 (when present) is encoded by the nucleotide sequence SEQ ID NO: 192, QsDMOT9 (when present) is encoded by the nucleotide sequence SEQ ID NO: 195, QsDMOT4 is encoded by the nucleotide sequence SEQ ID NO: 198, AtUXE (when present) is encoded by the nucleotide sequence SEQ ID NO: 201, AtUAM1 (when present) is encoded by the nucleotide sequence SEQ ID NO: 210, QsArafT2 (when present) is encoded by the nucleotide sequence SEQ ID NO: 234, AnNpgA (when present) is encoded by the nucleotide sequence SEQ ID NO: 239, QsCCL (when present) is encoded by the nucleotide sequence SEQ ID NO: 180 and AstLovF-TE (when present) is encoded by the nucleotide sequence SEQ ID NO: 236.

The method of any one of clauses 1 to 38, 42 to 45, 47 to 50, 52 to 61, 63 to 72, 74 to 80, 82 to 86, 88 to 91, 93 to 100, 102 to 108, 110 to 115, 118 to 121, 123 to 126, 128 to 139, 141 to 146, 148 to 154, 156 to 160, 162 to 168, 170 to 177, 179, 180, 182 to 188, 190 to 193, 195 to 198, 200, 201, 203, 204, and 206 to 213, wherein the method comprises the further step of culturing the yeast to allow production of QA, respective UDP-sugars, and/or respective QA derivatives.

The method of clause 214, wherein the culturing step ranges from 2 to 6 days.

The method of clause 215, wherein the culturing step is about 3 days.

The method of any one of clauses 1 to 38, 42 to 45, 47 to 50, 52 to 61, 63 to 72, 74 to 80, 82 to 86, 88 to 91, 93 to 100, 102 to 108, 110 to 115, 118 to 121, 123 to 126, 128 to 139, 141 to 146, 148 to 154, 156 to 160, 162 to 168, 170 to 177, 179, 180, 182 to 188, 190 to 193, 195 to 198, 200, 201, 203, 204, and 206 to 216, or the yeast of any one of clauses 39, 40, 46, 51, 62, 73, 81, 87, 92, 101, 109, 116, 117, 122, 127, 140, 147, 155, 161, 169, 178, 181, 189, 194, 199, 202, and 205 to 213, wherein the heterologous genes are overexpressed under inducible promoters.

The method, or the yeast, of clause 217, wherein induction is for 2 to 5 days, and yeasts are cultured for 2 to 5 more days.

The method of any one of clauses 1 to 38, 42 to 45, 47 to 50, 52 to 61, 63 to 72, 74 to 80, 82 to 86, 88 to 91, 93 to 100, 102 to 108, 110 to 115, 118 to 121, 123 to 126, 128 to 139, 141 to 146, 148 to 154, 156 to 160, 162 to 168, 170 to 177, 179, 180, 182 to 188, 190 to 193, 195 to 198, 200, 201, 203, 204, and 206 to 218, wherein the method further comprises the step of isolating UDP-sugars, C3-glycosylated QA derivatives, C28-glycosylated QA derivatives or acylated and glycosylated QA derivatives.

QA obtained according to the method of any one of clauses 1 to 38.

C3-glycosylated QA derivatives, C28-glycosylated QA derivatives or acylated and glycosylated QA derivatives obtained according to the method of clause 219.

The use of the QA derivatives of clause 221 as an adjuvant.

The use of clause 222, wherein the adjuvant is a liposomal formulation.

The use of clause 222 or clause 223, wherein the adjuvant comprises a TLR4 agonist.

The use of clause 224, wherein the TLR4 agonist is 3D-MPL.

An adjuvant composition comprising QS-21-Xyl according to clause 201 and/or QS-21-Api according to clause 203.

An isolated β-amyrin synthase (SvBAS) according to SEQ ID NO: 13.

An isolated β-amyrin synthase (QsBAS) according to SEQ ID NO: 15.

An isolated CYP C16 oxidase (QsC28C16) according to SEQ ID NO: 23.

An isolated CYP C16 oxidase (SvC16) according to SEQ ID NO: 26.

An isolated CYP C23 oxidase (SvC23-1) according to SEQ ID NO: 32.

An isolated CYP C23 oxidase (SvC23-2) according to SEQ ID NO: 35.

An isolated CYP C28 oxidase (SvC28) according to SEQ ID NO: 44.

An isolated Cytochrome b5 protein (Qsb5) according to SEQ ID NO: 55.

An isolated Cytochrome b5 protein (Svb5) according to SEQ ID NO: 61.

An isolated UDP-GlcA transferase (SvCslG) according to SEQ ID NO: 76.

An isolated MSBP protein (SvMSBP1) according to SEQ ID NO: 67.

An isolated MSBP protein (SvMSBP2) according to SEQ ID NO: 70.

An isolated MSBP protein (QsMSBP1) according to SEQ ID NO: 73.

An isolated UDP-glucose-4,6-dehydratase (SvUG46DH) according to SEQ ID NO: 87.

An isolated UDP-4-keto-6-deoxy-glucose reductase (SvNMD) according to SEQ ID NO: 90.

An isolated UDP-Galactose transferase (SvGalT) according to SEQ ID NO: 98.

An isolated UDP-Fucose transferase (SvFucT) according to SEQ ID NO: 96.

An isolated UDP-Xylose transferase (SvC3XylT) according to SEQ ID NO: 100.

An isolated UDP-Arabinofuranose transferase (QsArafT2) according to SEQ ID NO: 229.

An isolated UDP-glucose dehydrogenase (AtUGD_{A101L}) according to SEQ ID NO: 108.

An isolated UDP-Xylose transferase (QsC28XylT4-3aa) according to SEQ ID NO: 131.

An isolated UDP-Xylose transferase (QsC28XylT4-6aa) according to SEQ ID NO: 134.

An isolated UDP-Xylose transferase (QsC28XylT4-9aa) according to SEQ ID NO: 137.

An isolated UDP-Xylose transferase (QsC28XylT4-12aa) according to SEQ ID NO: 140.

An isolated UDP-Xylose transferase (SUMO-QsC28XylT4) according to SEQ ID NO: 143.

An isolated UDP-Xylose transferase (TrXA-QsC28XylT4) according to SEQ ID NO: 145.

An isolated UDP-Xylose transferase (MBP-QsC28XylT4) according to SEQ ID NO: 147.

An isolated UDP-Xylose transferase (QsC28XylT3-3×GGGS-QsC28XylT4) according to SEQ ID NO: 149.

An isolated type I polyketide synthase (AstLovF-TE) according to SEQ ID NO: 235.

### Examples

The genotypes of YL and SC yeast strains used in the Examples described below are provided in Table 3 and Table 4, respectively. Yeast engineering was carried out as described in Example 5 below (unless stated otherwise). Heterologous gene expression in yeast was carried out using nucleotide sequences that have been codon-optimized in order to increase the production of the corresponding protein. It is to be understood that codon optimization does not affect the amino acid sequence of the protein which is overexpressed. Heterologous genes have been integrated into the genome of the different yeast strains (as indicated), unless stated otherwise, under galactose-inducible promoters. After 2 days of culturing, expression of the heterologous genes has been induced with galactose added to the culture medium. 3 days post-induction, the production of sugars, QA precursors, QA, and acylated and/or glycosylated QA derivatives (as indicated) has been assessed by analysing their presence, by liquid chromatography-mass spectrometry (LC-MS) detection (as described in Example 6 below), after extraction of the yeast culture medium (as described in Example 5 below), unless stated otherwise.

### Example 1—Quillaic Acid (QA) Biosynthesis

### Production of the β-Amyrin Precursor

A previously developed mevalonate-overproducing strain, Jwy601, a CEN.PK2 based Saccharomyces cerevisiae strain was chosen as a parent strain (Wong et al. 2018). Jwy601 has been engineered to overexpress genes encoding β-amyrin synthases (BAS) of different plant origins by genome integration and the respective engineered yeast strains have been tested for their ability to convert 2,3-oxido-squalene into β-amyrin by analysing the presence of β-amyrin by gas chromatography-mass spectrometry (GC-MS) (using a standard commercially available).

### Results

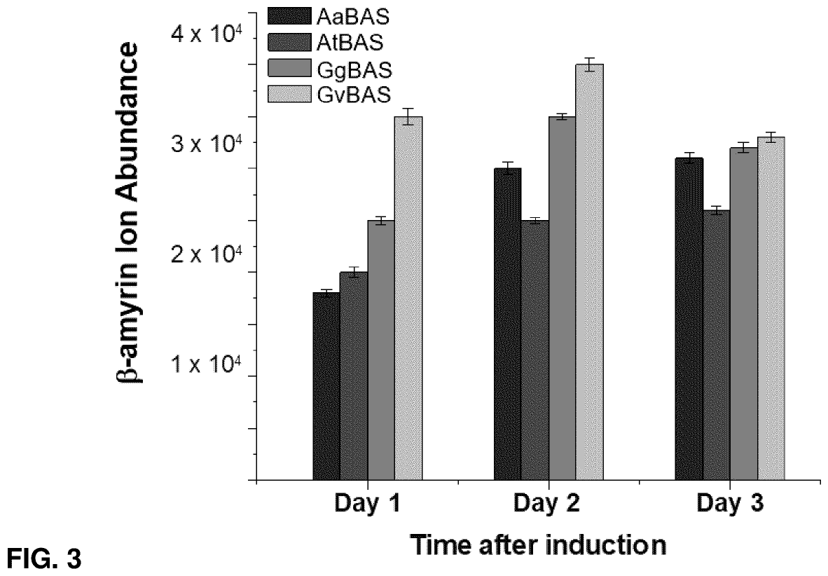

BAS from Artemisia annua (Aa) (named ‘AaBAS’ enzyme—SEQ ID NO: 1 encoded by SEQ ID NO: 3), Arabidopsis thaliana (At) (named ‘AtBAS’ enzyme—SEQ ID NO: 4 encoded by SEQ ID NO: 6), Glycyrrhiza glabra (Gg) (named ‘GgBAS’ enzyme—SEQ ID NO: 7 encoded by SEQ ID NO: 9), Gypsophila vaccaria (Gv) (named ‘GvBAS’ enzyme—SEQ ID NO: 10 encoded by SEQ ID NO: 12) have been tested. The BAS homolog from G. vaccaria yielded the highest production of β-amyrin (see FIG. 3). The yeast strain engineered with GvBAS (MLY-01) was therefore selected for further engineering as described below.

### Production of QA and Production Optimization

### Production of QA Precursors in Yeast

MLY-01 has been further engineered to co-express different cytochrome P450 (CYP) oxidases (C16, C23 and C28 oxidases) with a cytochrome P450 reductase (CPR) of different plant origins via sequential integration into the yeast genome. The production of QA and QA precursors has been analysed (using respective standards commercially available, e.g. from Merck, as a reference) by LC-MS in the yeast strains engineered with the following combination of enzymes:
A CPR from A. thaliana (named ‘AtATR1’—SEQ ID NO: 49 encoded by SEQ ID NO: 51)
A C16 oxidase from Bupleurum falcatum [CYP716Y1] (named ‘BfC16’—SEQ ID NO: 17 encoded by SEQ ID NO: 19)
A C23 oxidase from Medicago truncatula [CYP72A68] (named ‘MtC23’—SEQ ID NO: 38 encoded by SEQ ID NO: 40)
A C28 oxidase from Medicago truncatula [CYP716A12] (named ‘MtC28’—SEQ ID NO: 46 encoded by SEQ ID NO: 48)

### Results

Hederagenin and gypsogenin (QA precursors) were detectable. In addition, the pic obtained at about 10 min demonstrated the presence of QA at trace amount (<1 mg/L) (data not shown here, but data disclosed in FIG. 3 of WO 20/26354). These results confirm the functional relevance and activity of the CPR and CYP oxidases expressed in yeast and their ability to produce QA, when co-expressed in yeast.

CYP oxidases of alternative plant origins have been additionally tested. MLY-01 has been further engineered to co-express homologs CYP oxidases from Q. saponaria, together with the above AtAtr1, via sequential integration into the yeast genome, as follows:
A C16 oxidase [CYP716A297] (named ‘QsC16’—SEQ ID NO: 20 encoded by SEQ ID NO: 22)
A C23 oxidase [CYP714E52] (named ‘QsC23’—SEQ ID NO: 29 encoded by SEQ ID NO: 31),
A C28 oxidase [CYP716A24] (named ‘QsC28’—SEQ ID NO: 41 encoded by SEQ ID NO: 43)

In some experiments, the cytochrome b5 protein from Q. saponaria (named ‘Qsb5′-SEQ ID NO: 55 encoded by SEQ ID NO: 57) and/or the membrane steroid-binding protein from S. vaccaria (named ‘SvMSBP1’—SEQ ID NO: 67 encoded by SEQ ID NO: 69) have been further co-expressed (see also below Sections 1.2.4 and 1.2.5).

The production of QA and QA precursors has been analysed (using respective standards commercially available, e.g. from Merck, as a reference) by LC-MS in the yeast strains engineered with the following combinations of enzymes:
AtATR1-QsC28 (YL-1)
AtATR1-QsC28-QsC23-Qsb5 (YL-3)
AtATR1-QsC28-QsC23-Qsb5-QsC28C16 (YL-4)
AtATR1-QsC28-QsC23-Qsb5-QsC28C16-SvMSBP1 (YL-6)
AtATR1 (2 copies)-QsC28 (2 copies)-QsC23-Qsb5-QsC28C16 (YL-8)
AtATR1 (2 copies)-QsC28 (2 copies)-QsC23 (2 copies)-Qsb5 (2 copies)-QsC28C16 (2 copies)-SvMSBP1 (2 copies) (YL-10)

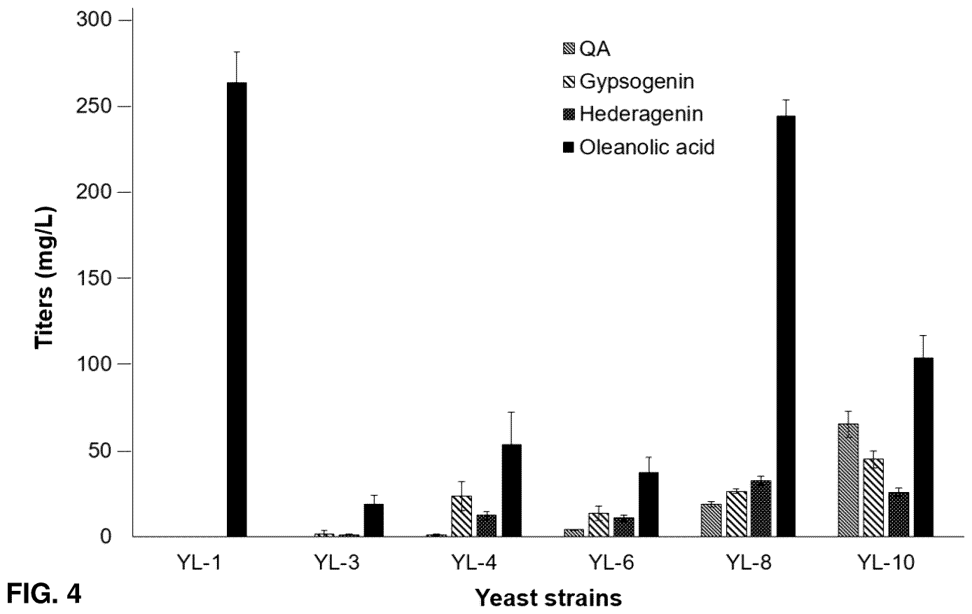

The data are provided in Table 2 below and the results are presented in the form of a graph in FIG. 4.

TABLE 2
Calculated titers of QA and QA precursors
(in mg/L) in engineered YL strains

| | Oleanolic acid | Hederagenin | Gypsogenin | QA |
|---|---|---|---|---|
| YL-1 | 263.38 | | | |
| YL-3 | 18.9 | 1.29 | 1.81 | |
| YL-4 | 23.57 | 11.78 | 12.49 | 1.1 |
| YL-6 | 37.51 | 10.8 | 13.58 | 4.04 |
| YL-8 | 244.03 | 32.51 | 26.3 | 18.85 |
| YL-10 | 104.07 | 25.8 | 44.91 | 65.22 |

### Results

As shown in FIG. 4 and in Table 2, while AtATR1 (the CPR reductase) alone was sufficient to facilitate C28 oxidation to carboxylic acid, leading to the production of 263.4 mg/L oleanolic acid (YL-1), C23 oxidation required Q. saponaria cytochrome b5 (Qsb5) for the hydroxy oxidation to an aldehyde functional group in gypsogenin (YL-3).

The additional co-expression of QsC16 (together with AtATR1, QsC23 and QsC28) did not result into QA production (data not shown), indicating that no oxidation at the C16 position happened, suggesting that QsC16 was non-functional.

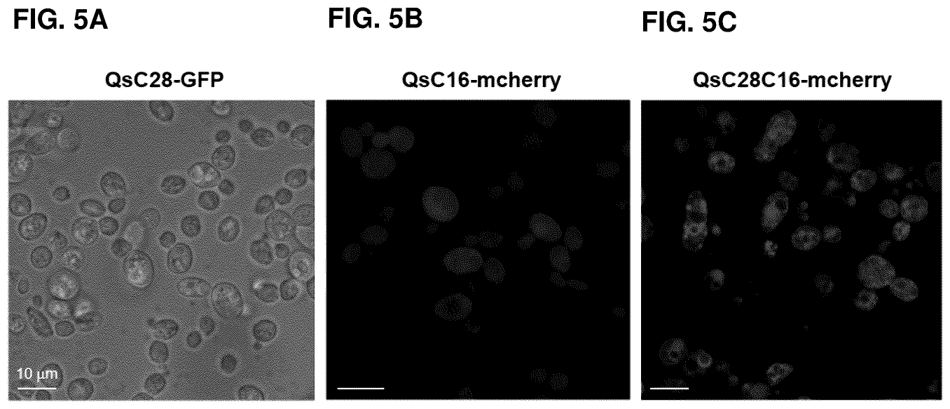

Subcellular localization studies revealed that, unlike other CYP oxidases, the C-terminally mcherry-tagged QsC16 oxidase is cytosolic, despite the presence of a predicted transmembrane domain at the N-terminus of the C16 oxidase. The confocal microscopy images obtained show that QsC18-GFP is localized in the endoplasmic reticulum (ER) membrane (FIG. 5, left image), while QsC16-mcherry is localized in the cytosol (FIG. 5, middle image).

In order to test the hypothesis that the lack of activity of QsC16 was due to inappropriate localization in yeast, the 22-amino acid predicted transmembrane domain of QsC28 was fused to the N-terminus of QsC16 (named ‘QsC28C16’—SEQ ID NO: 23 encoded by SEQ ID NO: 25), anchoring it to the ER membrane (FIG. 5, right image) where the CPR, the other CYP oxidases, as well as the terpene substrate, β-amyrin, are co-localized (data not shown).

When co-expressing QsC28C16 (instead of QsC16) in YL-4, QA was detected and produced at 1.1 mg/L (see Table 2 and FIG. 4).

The further co-expression of SvMSBP1, in YL-6, resulted into an increased global oxidation efficiency leading to an improved QA production (see Table 2 and FIG. 4). While the total titer of QA precursors remained consistent, the production of the final oxidation product (QA) was increased by 4-fold (4 mg/L) upon the co-expression of SvMSBP1, which co-localized with both QsC28 and QsC23 oxidases in the ER membrane (data not shown).

The simultaneous overexpression of 2 copies of QsC28 and 2 copies of AtATR1, in YL-8, led to an 8-fold increase in QA (18.9 mg/L) (see Table 2 and FIG. 4).

An additional second copy of all enzymes, in YL-10, led to a further optimized production of QA (65.2 mg/L) (see Table 2 and FIG. 4).

Gene Discovery in S. vaccaria—CYP Oxidases

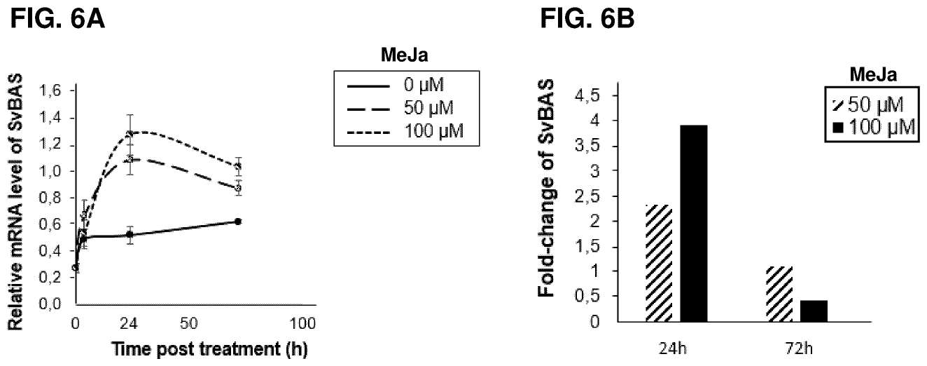

Leaves and flowers of S. vaccaria (Sv) have been treated with 0, 50 μM or 100 μM methyl jasmonate (Meja) for 72 h. The expression level of β-amyrin synthase mRNA has been analyzed (in leaves) (see FIG. 6A) and the fold-change of β-amyrin synthase mRNA expression induced by MeJa at 50 μM or 100 μM has been compared to 0 μM at 24 h and 72 h in flowers (see FIG. 6B). A neighbor-joining tree (1,000 bootstrap replicates) of cytochrome P450 (CYP) oxidases acting on triterpenoids from other plants and CYP candidates identified from S. vaccaria transcriptome (see also Section 1.2.4 below) is shown in FIG. 6C. Gene names of CYPs from S. vaccaria newly identified are labelled with an asterisk (*). Gene names of CYPs from S. vaccaria newly identified that are co-expressed with p amyrin synthase (BAS) are highlighted in boxes.

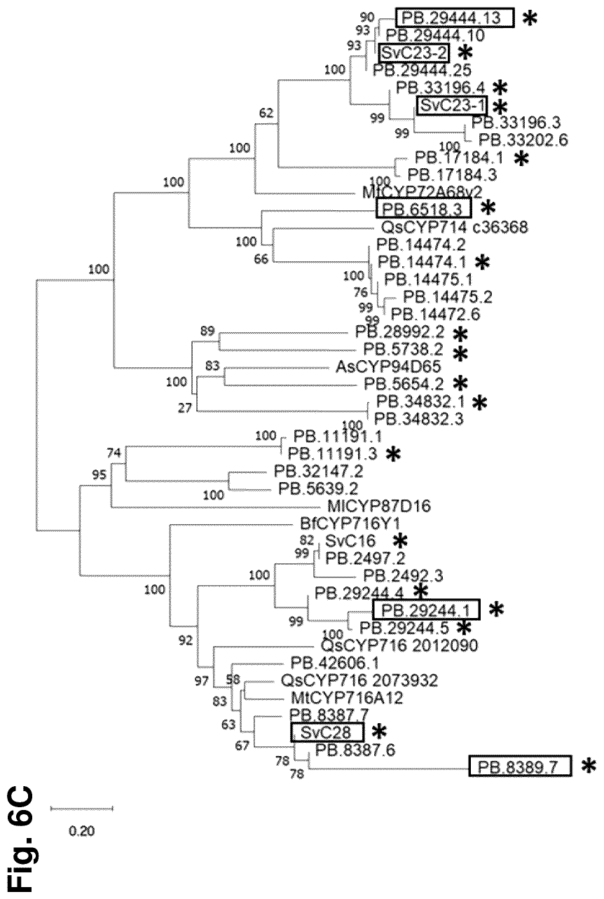

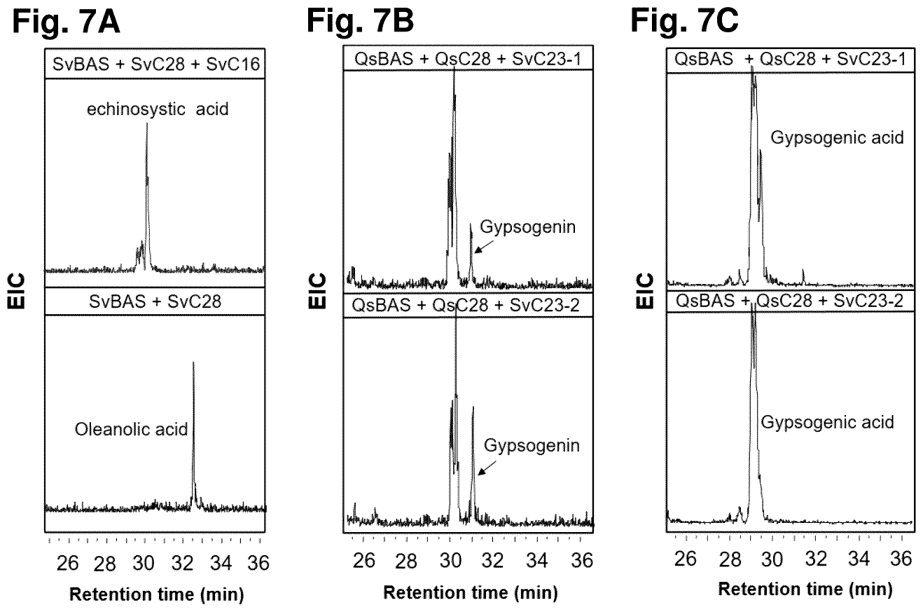

The functional relevance and activity of ‘SvC16′, ‘SvC23-1′, ‘Sv23-2’ and ‘SvC28′ (as named in FIG. 6C) has been tested in N. benthamiana, in combination with β-amyrin synthases (BAS) of different plant origins. The following enzymes have been transiently expressed in Nicotiana benthamiana, in different combinations (as indicated in FIG. 7):
A β-amyrin synthase from S. vaccaria (BAS) (named ‘SvBAS'—SEQ ID NO: 13 encoded by SEQ ID NO: 14)
A R-amyrin synthase (BAS) from Q. Saponaria (named ‘QsBAS'—SEQ ID NO: 15 encoded by SEQ ID NO: 16)
A C16 oxidase from S. vaccaria (named ‘SvC16’—SEQ ID NO: 26 encoded by SEQ ID NO: 27)
A C28 oxidase from S. vaccaria (named ‘SvC28’—SEQ ID NO: 44 encoded by SEQ ID NO: 45)
A C23 oxidase from S. vaccaria (named ‘SvC23-1’—SEQ ID NO: 32 encoded by SEQ ID NO: 33)
A C23 oxidase from S. vaccaria (named ‘SvC23-2’—SEQ ID NO: 35 encoded by SEQ ID NO: 36)

The production of QA precursors has been analyzed (using respective standards commercially available, e.g. from MCE, Chemcruz and TCI, as a reference) by LC-MS.

### Results

Results are shown in FIG. 7.

Echinocystic acid and oleanolic acid were detected when co-expressing SvBAS, SvC28 and SvC16 (FIG. 7A).

Gyspogenin was detected when co-expressing QsBAS, QsC28 and each of SvC23-1 or SvC23-2 (FIG. 7B)

Gypsogenic acid was detected when co-expressing QsBAS, QsC28 and each of SvC23-1 or SvC23-2 (FIG. 7C)

These results confirm the functional relevance and activity of QsBAS, and the newly identified SvC16, SvC23-1, SvC23-2 and SvC28 oxidases, as well as their ability to produce QA precursors, when co-expressed in N. benthamiana.

QA Production in Yeast Using S. vaccaria Genes SvC16, SvC23-1 and SvC23-2

MLY-01 has been transformed with the following plasmids: pESC-TRP-SepGAL2-SvC16, pGAL10-AtAtr1, pGAL1-QsC28, pGAL7-SvC23-1 or pESC-TRP-SepGAL2-SvC16, pGA10-AtAtr1, pGAL1-QsC28, pGAL7-SvC23-2. The production of QA and QA precursors has been analyzed (using respective standards commercially available, as a reference) by HPLC/LC-MS.

### Results

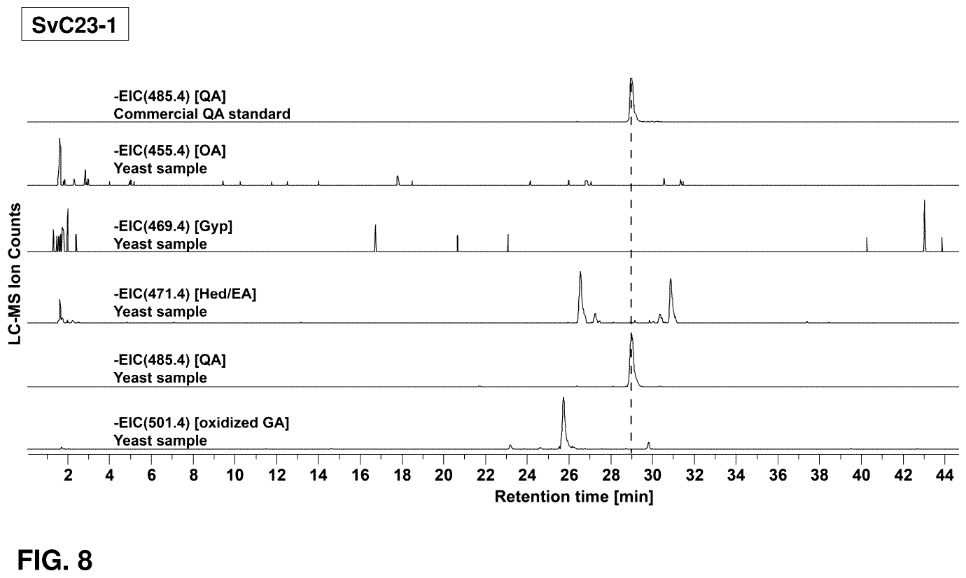

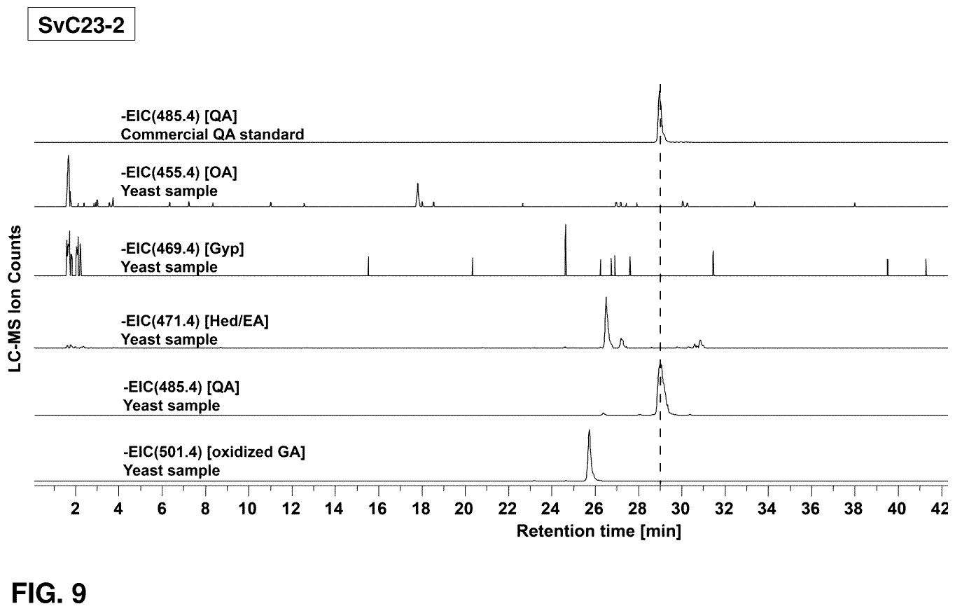

Both chromatograms in FIG. 8 and FIG. 9 show a peak exactly matching the exact m/z value and retention time of the commercial QA standard (dashed line).

Confocal microscopy images revealed that SvC16 is well-expressed and localizes properly in the endoplasmic reticulum (ER) of the yeast (data not shown), in contrary to OsC16 (see above Section 1.2.1).

These results confirm the functional relevance of SvC16, SvC23-1 and SvC23-2 oxidases, as well as their ability to produce QA, when co-expressed with AtATR1 and QsC28 in yeast.

Gene Discovery in S. vaccaria—MSBP Proteins

Genes encoding MSBP homologs to A. thaliana (At) have been identified in S. vaccaria (Sv) transcriptome by sequence similarity search using algorithm tblastn. Amino acid sequences of MSBPs from At (named ‘AtMSBP1’—SEQ ID NO: 63 and ‘AtMSBP2’—SEQ ID NO: 65) were submitted in a database of Sv transcriptome (prepared in-house) for a comparison with translated DNA sequences of all genes in the transcriptome. Similar sequences were selected based on sequence identity (last column of Table 3) and the significance of sequence match (third column of Table 3). The results are summarized in Table 3 below.

TABLE 3
Arabidopsis thaliana (At) MSBP homologs in S. vaccaria (Sv)

| | | Sv Subject | | |
|---|---|---|---|---|
| | Sv | length | | % |
| Query | Subject* | (bp) | E-value | Identity |
| AtMSBP1 | PB.394.2 | 695 | 6.58E−70 | 61.86 |
| SEQ ID NO: 63 | PB.392.2 | 930 | 6.12E−69 | 61.86 |
| | PB.394.1 | 1000 | 1.57E−68 | 61.86 |
| | PB.393.1** | 1203 | 8.72E−67 | 58.636 |
| | PB.16084.1 | 960 | 3.93E−46 | 52.381 |
| | PB.16084.2** | 1429 | 9.84E−45 | 52.381 |
| | PB.38275.1 | 1014 | 1.04E−13 | 40.244 |
| AtMSBP2 | PB.394.2 | 695 | 1E−80 | 72.105 |
| SEQ ID NO: 65 | PB.393.1** | 1203 | 7.7E−80 | 71.429 |
| | PB.392.2 | 930 | 1.08E−79 | 72.105 |
| | PB.394.1 | 1000 | 1.74E−79 | 72.105 |
| | PB.16084.1 | 960 | 1.31E−53 | 53.846 |
| | PB.16084.2** | 1429 | 4.76E−52 | 53.846 |
| | PB.38275.1 | 1014 | 1.6E−18 | 38.614 |

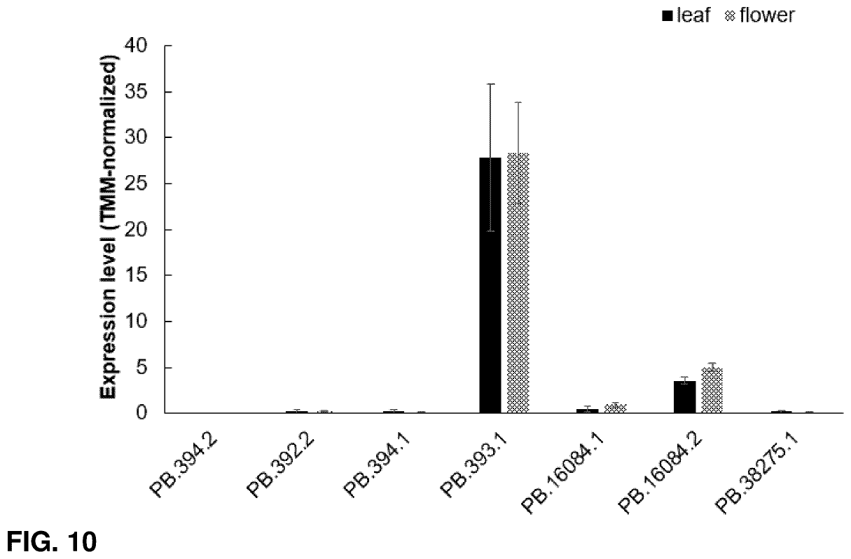

*Transcript names
**The longest 2 sequences (also showing the highest expression level in leaves and flowers, as shown in FIG. 10) were selected for functional test in yeast (as described in the below Section 1.2.5).

The average expression levels of the different homologs identified in Table 3 has been analysed in leaves and flowers of S. vaccaria (see FIG. 10).

QA Production in Yeast Using Homologs MSBP from S. vaccaria

The functional relevance and activity of the transcripts PB.393.1 and PB.16084.2 has been tested for their ability to increase the oxidation efficiency and improve QA production in yeast. Respective corresponding proteins have been named ‘SvMSBP1′ (SEQ ID NO: 67 encoded by SEQ ID NO: 69) and ‘SvMSBP2′ (SEQ ID NO: 70 encoded by SEQ ID NO: 72) and have been integrated into the genome of yeasts engineered to produce QA, as follows:
AtATR1/QsC28/QsC23/Qsb5/QsC28C16/SvMSBP1 (YL-6)
AtATR1/QsC28/QsC23/Qsb5/QsC28C16/SvMSBP2
AtATR1/QsC28/QsC23/Qsb5/QsC28C16/QsMSBP1
AtATR1/QsC28/QsC23/Qsb5/QsC28C16 (YL-4) has been used as a control

A homolog MSBP from Q. saponaria (named ‘QsMSBP1’—SEQ ID NO: 73 and encoded by SEQ ID NO: 75) has been tested as well. The production of QA and QA precursors has been analyzed (using respective commercial standards as a reference) by LC-MS. Results are presented in the form of a graph in FIG. 11.

### Results

As compared with YL-4 (which does not overexpress any MSBP protein), in yeasts overexpressing MSBP proteins (whether from S. vaccaria or from Q. saponaria), a significant increase in QA production was observed, with SvMSBP1 and SvMSBP2 performing better (see FIG. 11). SvMSBP1 was selected for further yeast engineering to produce C3-glycosylated QA derivatives (see Example 2 below), C28-glycosylated QA derivatives (see Example 3 below) and QS-21-Xyl and QS-21-Api (see Example 4 below).

### Conclusion

Using different heterologous enzymes (β-amyrin synthase, CYP oxidases, CYP reductase) and heterologous proteins (cytochrome b5 and MSBP proteins) from different plant origins (e.g. G. vaccaria, A. thaliana, Q. saponaria and S. vaccaria), in different combinations, the inventors have been able to reconstruct in yeast the metabolic pathway leading to the biosynthesis of QA, achieving, for the first time, the successful production of QA in yeast at about 65 mg/L.

### Example 2—C3-Glycosylated QA Derivatives Biosynthesis

### Production of UDP-Sugars Non-Native to Yeast (Glucuronic Acid, Xylose and Rhamnose)

### Glucuronic Acid (GlcA)

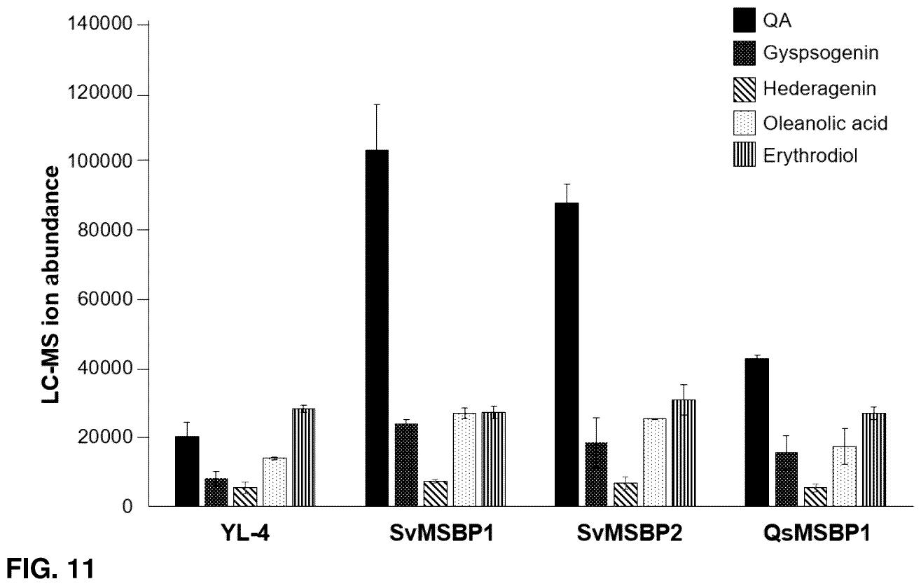

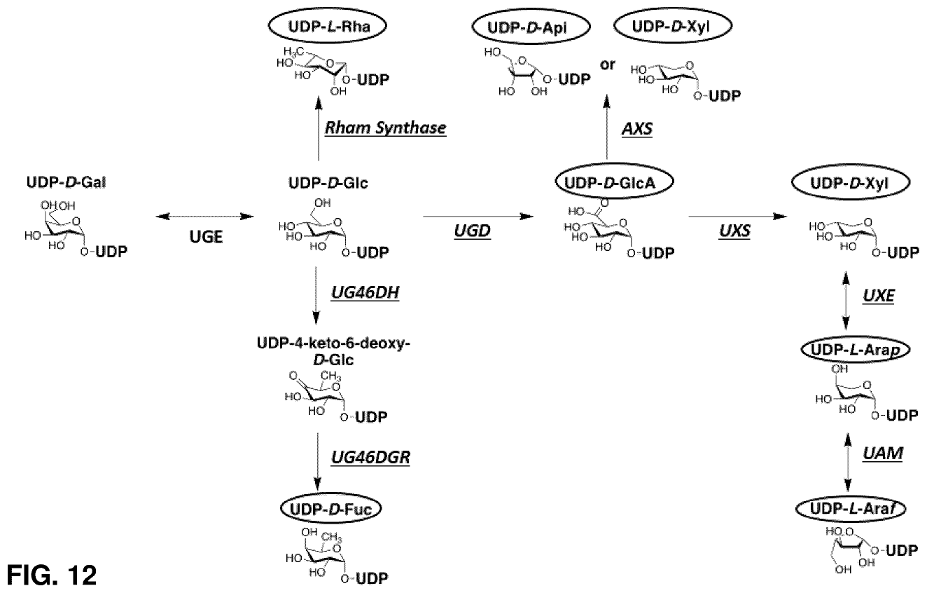

As shown in FIG. 12, UDP-GlcA is produced by a UDP-glucose dehydrogenase (UGD) from UDP-Glucose. A gene encoding a UDP-glucose dehydrogenase from A. thaliana (named ‘AtUGD’—SEQ ID NO: 84 encoded by SEQ ID NO: 86), has been integrated into the genome of the parent yeast strain CEN.PK2-1c to generate SC-1. The production of UDP-GlcA has been analyzed by LC-MS.

### Results

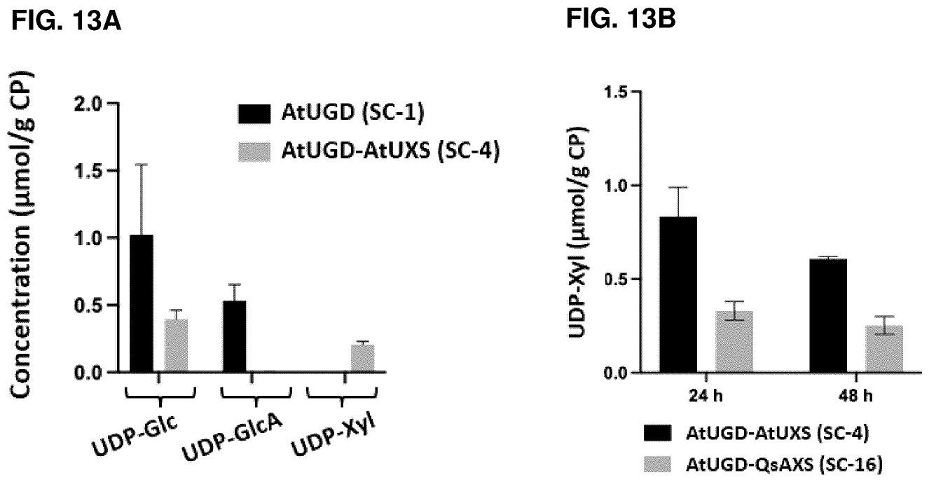

FIG. 13A shows that UDP-GlcA was detected by SC-1, confirming the functional activity of AtUGD when overexpressed in yeast.

### Xylose (Xyl)

As shown in FIG. 12, UDP-GlcA can be decarboxylated by a UDP-xylose synthase (UXS) to form UDP-Xyl. A UDP-xylose synthase from A. thaliana (named ‘AtUXS’—SEQ ID NO: 105 encoded by SEQ ID NO: 107) has been integrated into the genome of SC-1 (overexpressing AtUGD) to generate SC-4. As shown in FIG. 12, UDP-GlcA can also be decarboxylated by a UDP-apiose synthase (AXS) to form UDP-Xyl. A UDP-apiose synthase from Q. saponaria AXS (named ‘QsAXS'—SEQ ID NO: 113 encoded by SEQ ID NO: 115) has been integrated, together with AtUGD, into the genome of the parent yeast strain CEN.PK2-1c to generate SC-16. The production of UDP-Xyl has been analyzed by LC-MS.

### Results

The production of UDP-Xyl was detected in both SC-4 and SC-16 (see FIG. 13A and FIG. 13B), confirming the functional activity of AtUXS and QsAXS when overexpressed in yeast.

### Rhamnose (Rha)

The expression of the trifunctional AtRHM2 synthase enzyme from A. thaliana (named ‘AtRHM2’—SEQ ID NO: 102 encoded by SEQ ID NO: 104) has been investigated as a potential rhamnose synthase. AtRHM2 catalyzes the conversion from UDP-Glc directly to UDP-Rha via (i) the dehydration of UDP-Glc followed by (ii) the epimerization of the C3′ and C5′ positions to form UDP-4-keto-β-L-rhamnose and (iii) the reduction of UDP-4-keto-β-L-rhamnose to produce UDP-β-L-rhamnose. AtRHM2 has been integrated into the genome of the parent yeast strain CEN.PK2-1c to generate SC-17 and into the genome of SC-4 to generate SC-18. The production of UDP-Rha has been analyzed by LC-MS.

### Results

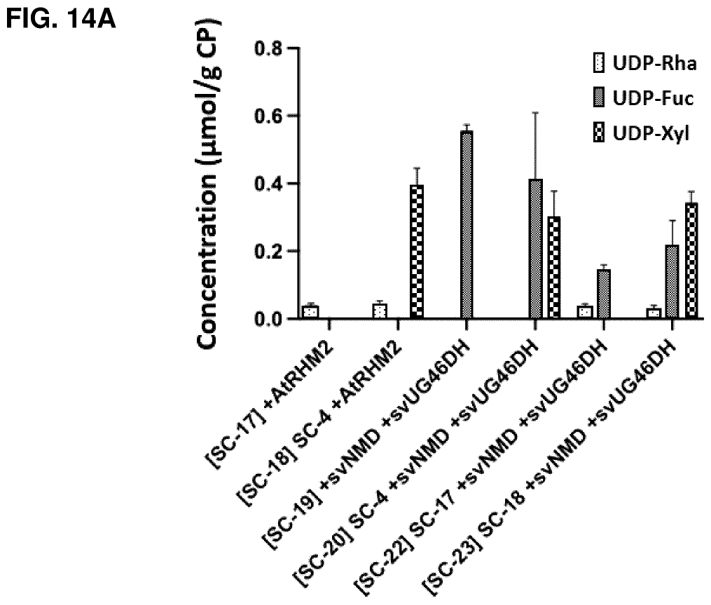

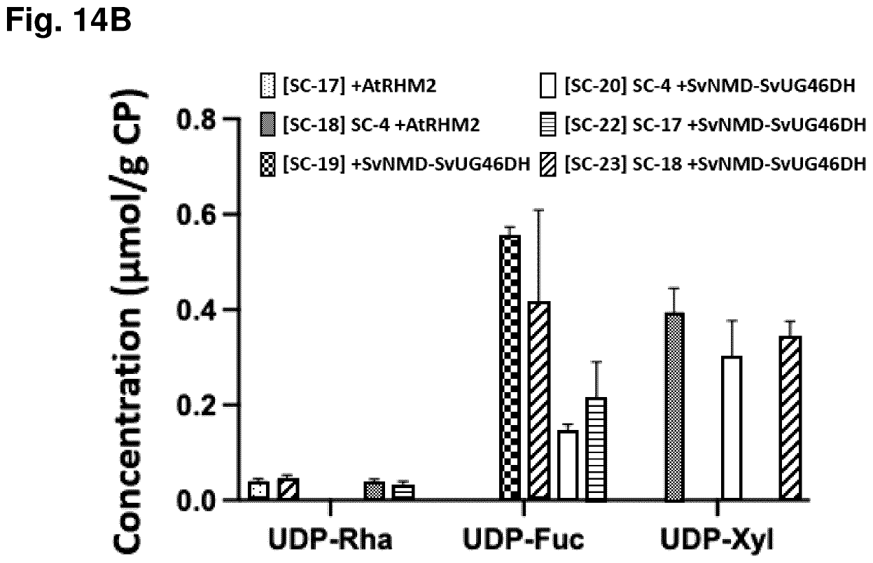

The production of UDP-Rha was detected in both SC-17 and SC-18 (see FIG. 14A and FIG. 14B), confirming the functional activity of ARHM2 when overexpressed in yeast.

### Production of QA-C3-GlcA

The same AtUGD as above has been integrated into the genome of YL-10 (producing QA), together with a glucuronic acid transferase (GlcAT) from Q. saponaria (named ‘QsCslG1’—SEQ ID NO: 78 encoded by SEQ ID NO: 80) or a second glucuronic acid transferase from Q. saponaria (named ‘QsCslG2’—SEQ ID NO: 81 encoded by SEQ ID NO: 83) to generate YL-11 and YL-12, respectively. Production of QA precursors as well as QA-C3-GlcA has been analyzed by LC-MS, using respective standards as a reference (QA-C3-GlcA standard corresponds to QAGlcpA, generated as described in WO 20/260475).

### Results

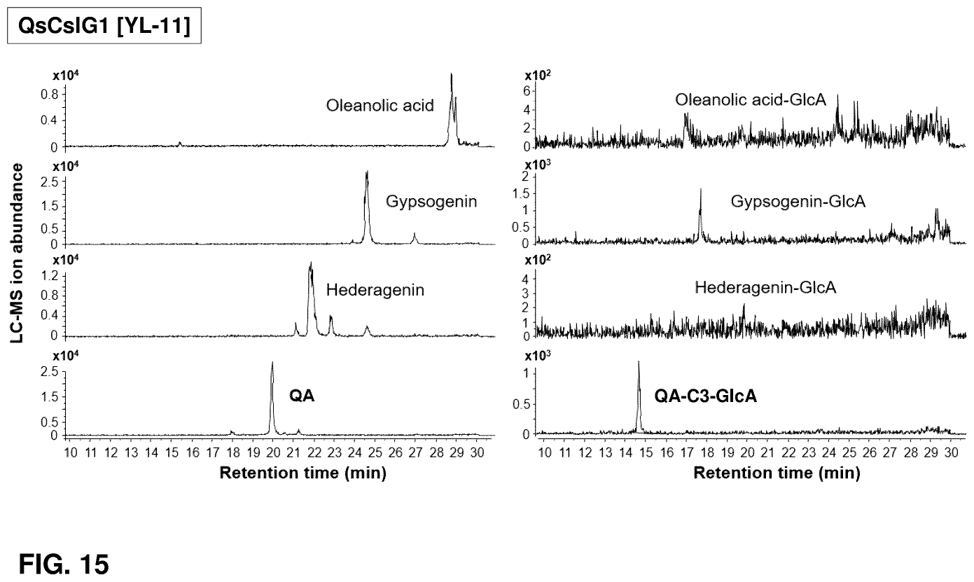

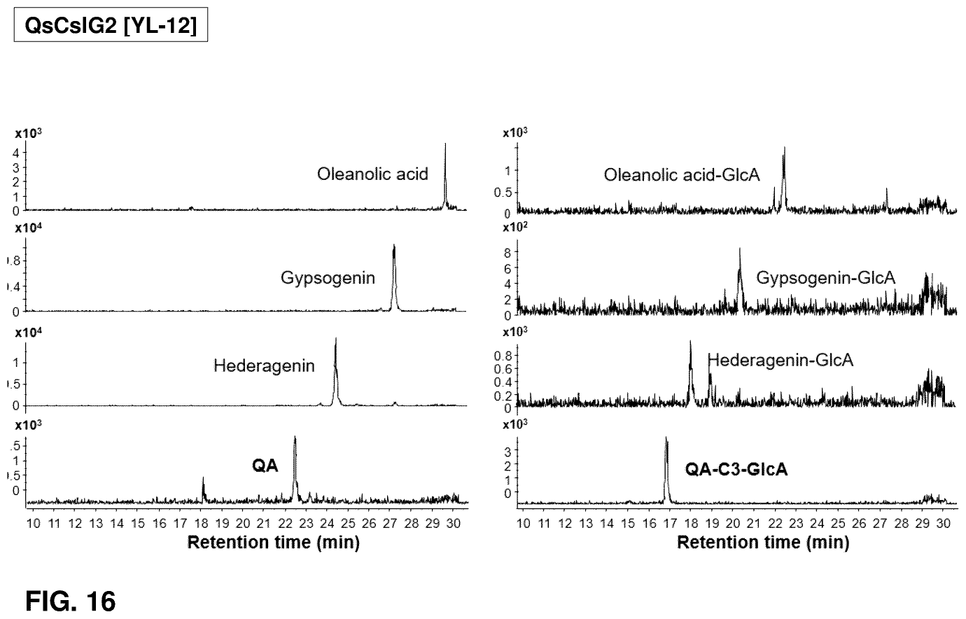

QA-C3-GlcA was detected in both YL-11 (overexpressing QsCslG1) and YL-12 (overexpressing QsCslG2) (see FIG. 15 and FIG. 16, respectively), confirming the functional activity of the two enzymes when overexpressed in yeast.

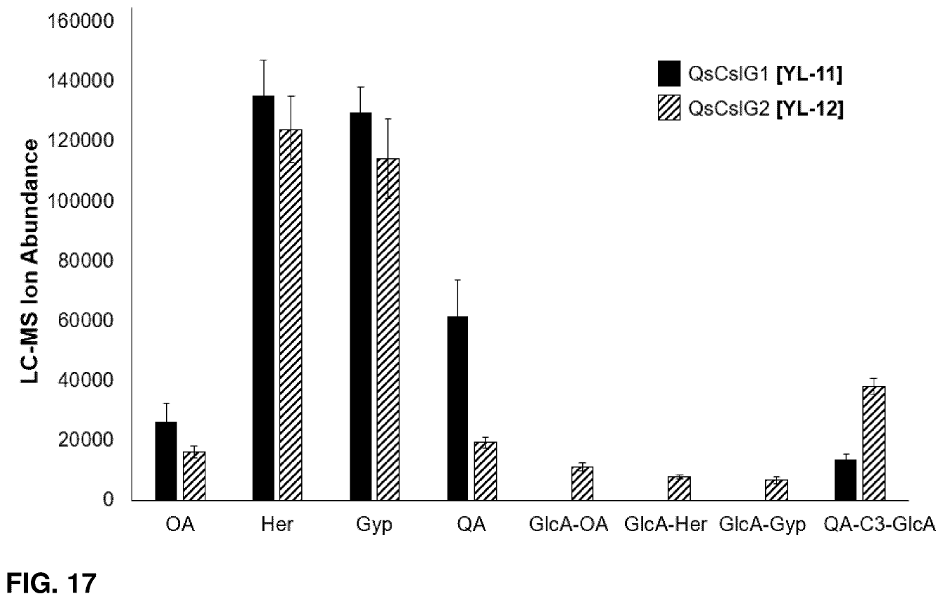

QsCslG1 is specific towards QA and does not glycosylate other precursors, while QsCslG2 enzyme is promiscuous and 3 times more reactive than CslG1 enzyme to produce GlcA-QA (10.2 mg/L and 3.9 mg/L, respectively) (see FIG. 17).

The inventors also identified in the transcriptome of S. vaccaria a novel gene encoding a CslG homolog enzyme (named ‘SvCslG’—SEQ ID NO: 76 encoded by SEQ ID NO: 77). The function of SvCslG as a GlcA transferase candidate has been confirmed using an in vitro enzymatic assay. QA (commercially available, e.g. from MedChemExpress) and UDP-GlcA, have been directly added into the reaction buffer together with a microsomepreparation of a yeast strain overexpressing SvCslG via plasmid expression. The production of QA and QA-C3-GlcA was analyzed by LC-MS.

### Results

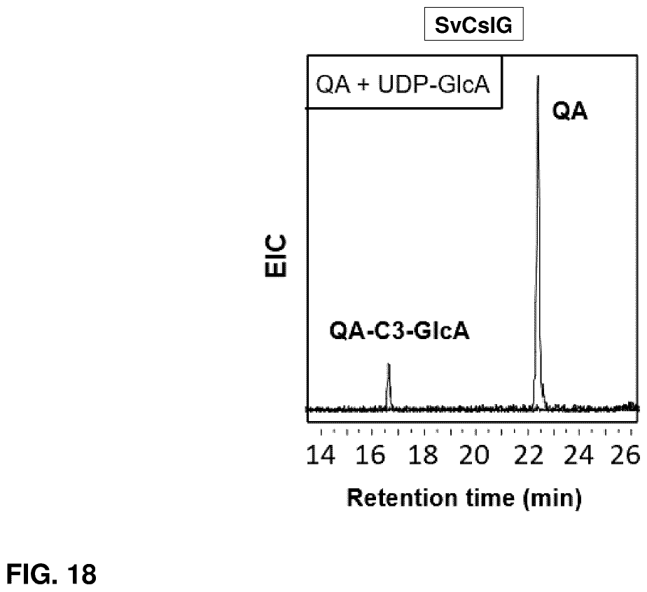

FIG. 18 shows that, in the presence of UDP-GlcA, a peak corresponding to QA-C3-GlcA was observed, indicating the ability of SvCslG to transfer UDP-GlcA to the C3 position of QA, confirming its functional relevance and activity when expressed in yeast.

### Production of QA-C3-GlcA-Gal

UDP-galactose is natively produced in yeast and therefore, no addition of a sugar synthase is necessary for this glycosylation step. A galactose transferase from Q. Saponaria (named ‘QsGalT’—SEQ ID NO: 116 and encoded by SEQ ID NO: 118) has been integrated into the genome of YL-12 to generate YL-13. The production of QA-C3-GlcA and QA-C3-GlcA-Gal has been analyzed (using respective standards) by LC-MS. QA-C3-GlcA standard and QA-C3-GlcA-Gal standard corresponds to ‘QAGlcpA’ and ‘QA-GlcpA-Galp’, respectively, generated as described in WO 20/260475.

### Results

FIG. 19 shows that the overexpression of QsGalT facilitates the glucuronidation step, possibly by complete conversion of QA-C3-GlcA to QA-C3-GlcA-Gal, and thus, pushing the reaction equilibrium towards further glycosylation. The production of QA-C3-GlcA-Gal achieved in YL-13 was 24.3 mg/L.

The inventors also identified in the transcriptome of S. vaccaria a novel gene encoding a galactose transferase candidate (named ‘SvGalT’—SEQ ID NO: 98 encoded by SEQ ID NO: 99). The function of SvGalT as a galactose transferase has been confirmed by transiently expressing SvCslG and SvGalT in N. benthamiana plants. Plants have been infiltrated with 40 μM of QA (commercially available, e.g. from MedChemExpress) 2 days after Agrobacterium tumefaciens infiltration. The production of QA-C3-GlcA and QA-C3-GlcA-Gal has been analyzed (using respective standards) by LC-MS. QA-C3-GlcA standard and QA-C3-GlcA-Gal standard correspond to ‘QAGlcpA’ and ‘QA-GlcpA-Galp’, respectively, generated as described in WO 20/260475.

### Results

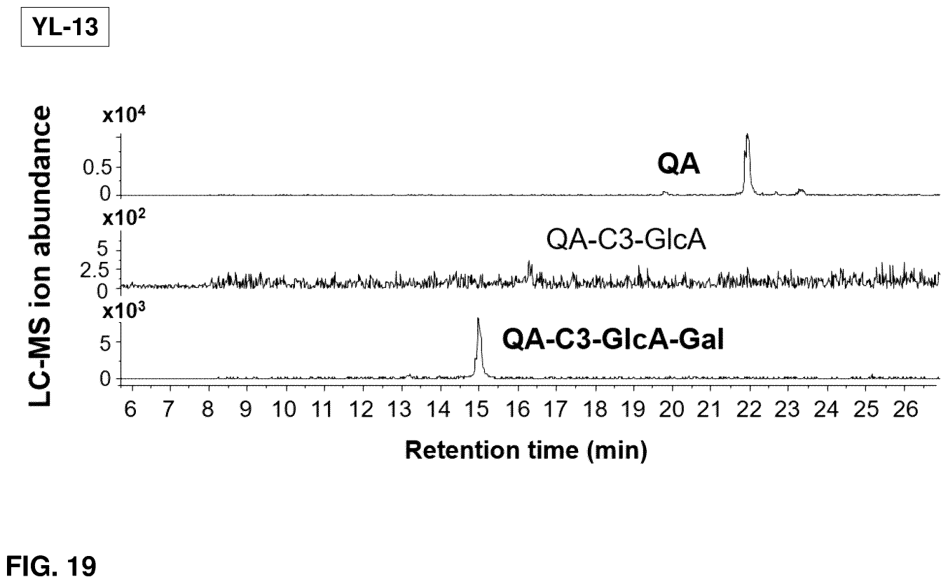

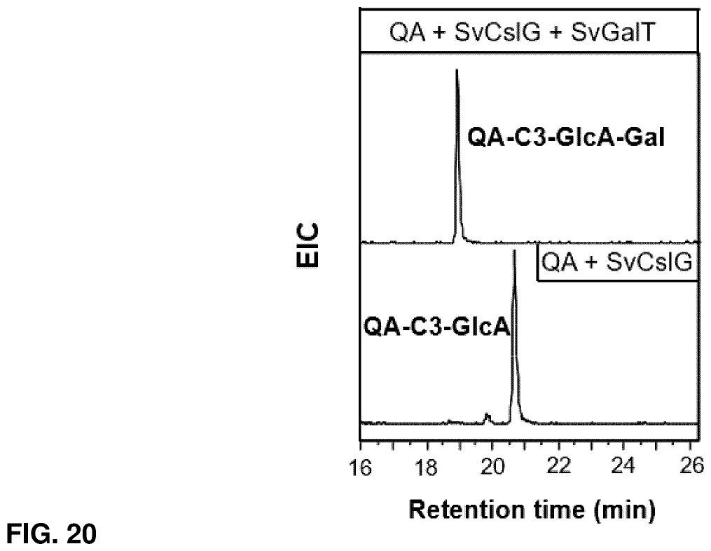

FIG. 20 shows that a peak corresponding to QA-GlcA-Gal was observed when co-expressing SvCslG and SvGalT, indicating the ability of SvGalT to transfer UDP-Gal to QA-GlcA, confirming its functional relevance and activity.

### Production of QA-C3-GlcA-Gal-Rha

The above AtRHM2 and a rhamnose transferase from Q. Saponaria (named ‘QsRhaT’-SEQ ID NO: 119 and encoded by SEQ ID NO: 121) have been integrated into the genome of YL-13 to generate YL-14. The production of QA-C3-GlcA and QA-C3-GlcA-Gal and QA-C3-GlcA-Gal-Rha has been analyzed (using respective standards) by LC-MS. QA-C3-GlcA standard, QA-C3-GlcA-Gal standard and QA-C3-GlcA-Gal-Rha correspond to ‘QAGlcpA’, ‘QA-GlcpA-Galp’, and ‘QA-GlcpA-Galp-Rhap’, respectively, generated as described in WO 20/260475.

### Results

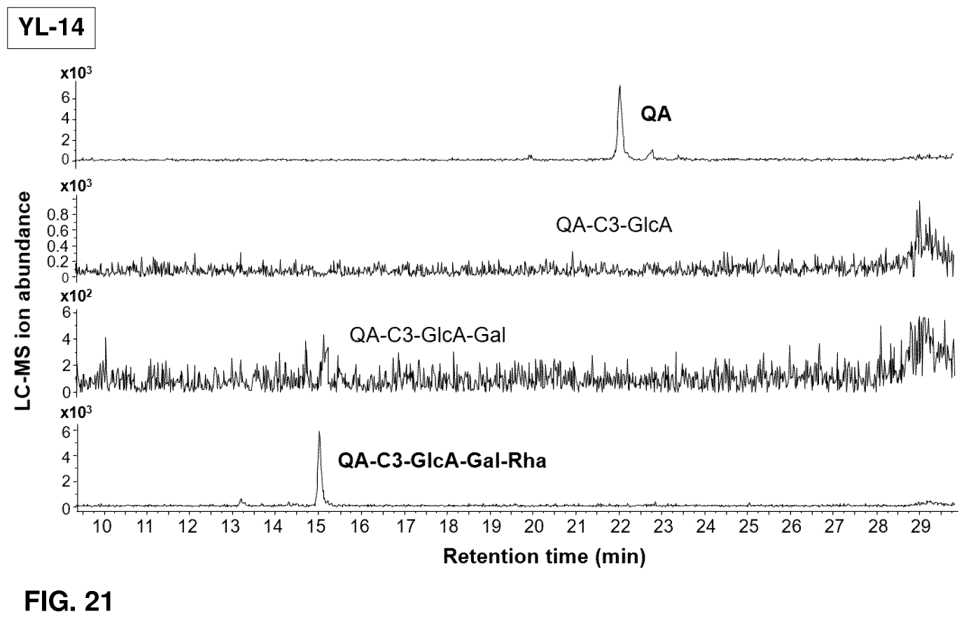

FIG. 21 shows that the co-expression of AtRHM2 and QsRhaT, together with AtUGD and OsGalT, resulted into the production of QA-C3-GlcA-Gal-Rha. The level achieved was 9.5 mg/L. No residual QA-GlcA-Gal was observed indicating that QsRhaT is highly efficient and catalyzes the complete conversion of QA-GlcA-Gal to QA-C3-GlcA-Gal-Rha.

### Production of QA-C3-GlcA-Ga-Xyl

The above AtUXS has been integrated into the genome of YL-12 (a yeast strain engineered to produce UDP-GlcA). Direct expression of AtUXS in the UDP-GlcA-producing strain led to the absence of any glycosylated molecule (data not shown), possibly due to insufficient UDP-GlcA production. This suggested that the downstream metabolite UDP-Xylose may act as an allosteric feedback inhibitor controlling the activity of UGD. This is confirmed in FIG. 13A showing that there was no detectable UDP-GlcA when AtUXS is being co-expressed with AtUGD.

### AtUGD Mutation

It has been reported that a point mutation A104L engineered in the human UGD homolog has led to a lower UDP-Xyl binding affinity. Therefore, as an attempt to alleviate the observed UGD inhibition induced by UDP-Xyl, mutation(s) were introduced into AtUGD in order to lower UDP-Xyl binding affinity. The protein sequence of AtUGD was aligned against that of the human UGD to identify the corresponding amino acid (data not shown), and a point mutation A101 L was introduced into AtUGD (AtUGD_{A101L}—SEQ ID NO: 108 encoded by SEQ ID NO: 109). AtUGD_{A101L}, has been integrated into the genome of YL-10 (yeast engineered to produce QA), together with the above QsCslG2, and OsGalT, as well as with a UDP-xylose transferase from Q. saponaria (QsC3XylT—SEQ ID NO: 122 encoded by SEQ ID NO: 124), to generate YL-15. The production of QA-C3-GlcA, QA-C3-GlcA-Gal and QA-C3-GlcA-Gal-Xyl has been analyzed (using respective standards) by LC-MS.

### Results

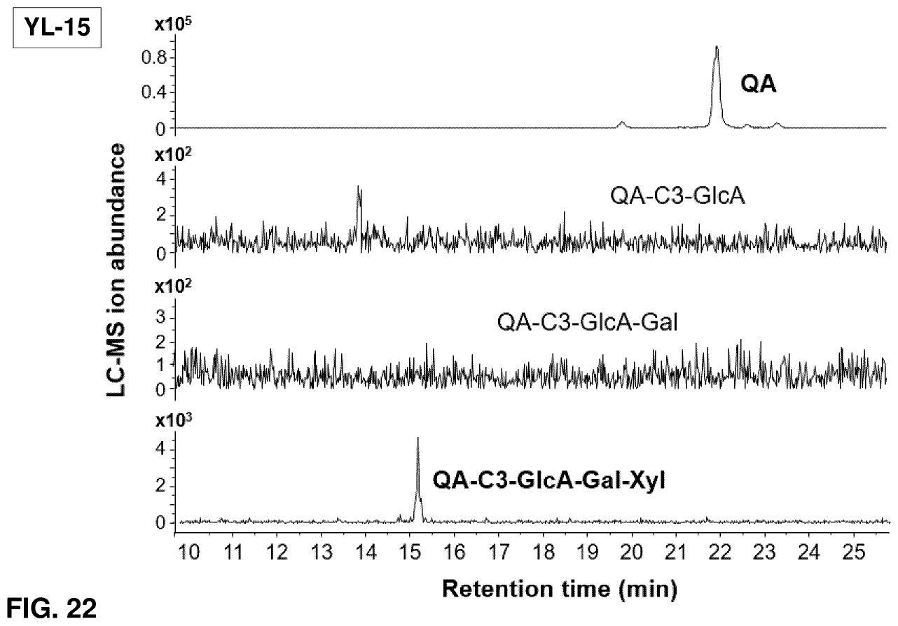

FIG. 22 shows that QA-C3-GlcA-Gal-Xyl was detected in YL-15, with a level achieved at 1 mg/L.

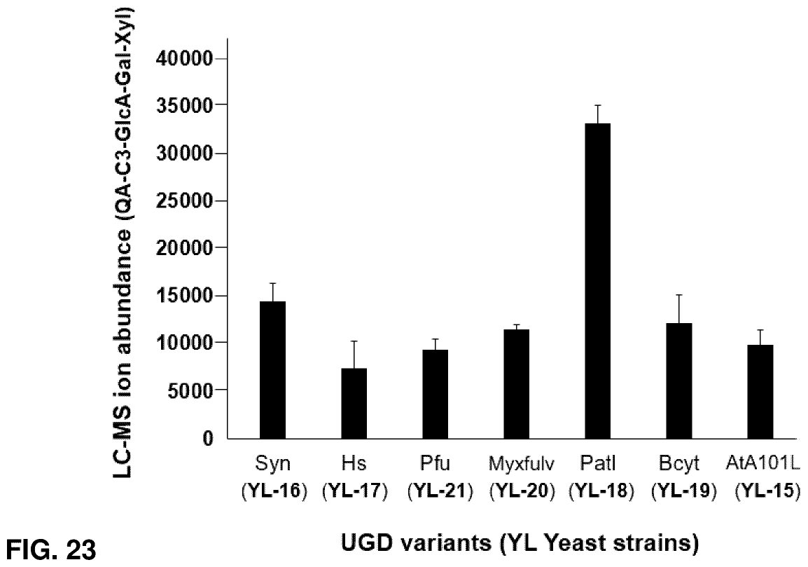

In order to investigate the varying degrees of UDP-Xyl inhibition on different UGDs, six homologs were selected across kingdoms to include those from Synechococcus sp. (Syn) (named ‘SynUGD’—SEQ ID NO: 154 encoded by SEQ ID NO: 156), Homo sapiens (Hs) (named ‘HSUGD_{104L}’—SEQ ID NO: 157 encoded by SEQ ID NO: 159), Paramoeba atlantica (Patl) (named ‘PatIUGD’—SEQ ID NO: 110 encoded by SEQ ID NO: 112), Bacillus cytotoxicus (Bcyt) (named ‘BcytUGD’—SEQ ID NO: 160 encoded by SEQ ID NO: 162), Corallococcus macrosporus (Myxfulv) (named ‘MyxfulvUGD’—SEQ ID NO: 163 encoded by SEQ ID NO: 165), Pyrococcus furiosus (Pfu) (named ‘PfuUGD’—SEQ ID NO: 166 encoded by SEQ ID NO: 168). The sequences of these homologs have been integrated into genome of YL-10 (a yeast strain engineered to produce QA), together with the above QsCslG2, QsGalT, AtUXS, and QsC3XylT, generating YL-16 to YL-21, respectively. The production of QA-C3-GlcA-Gal-Xyl has been analyzed by LC-MS (using respective standards). The results are presented in FIG. 23 in the form of a graph.

### Results

FIG. 23 shows that, while the production of QA-C3-GlcA-Gal-Xyl from other UGD enzymes was comparable with AtUGD_{A101L} (YL-15 being used as a control), PatIUGD (YL-18) yielded 3 times higher in production. Upon sequence alignment of PatIUGD with AtUGD, it was noticed that the A101L mutation of AtUGD is natively present in PatIUGD, which may increase its tolerance of UDP-Xyl (data not shown).

### Alternative UDP-GlcA Biosynthesis Pathway

UDP-GlcA can also be generated via the de novo salvage pathway or the myo-inositol oxidation pathway. Glucuronokinase (GlcAK) and UDP-sugar pyrophosphorylase (USP) convert free glucuronic acid to GlcA-1-phosphate and eventually the active UDP form of GlcA (UDP-GlcA). These enzymes are also responsible for the myo-inositol pathway starting with myo-inositol oxygenase (named ‘MIOX’). A GlcAK enzyme from A. thaliana (named ‘AtGlcAK’—SEQ ID NO: 169 encoded by SEQ ID NO: 171) and a USP from A. thaliana (named ‘AtUSP’—SEQ ID NO: 223 encoded by SEQ ID NO: 225) have been integrated into the genome of YL-15 to generate YL-22. The same GlcAK and AtUSP have been separately integrated, together with a MIOX from Thermothelomyces thermophilus (named ‘TtMIOX’—SEQ ID NO: 172 encoded by SEQ ID NO: 174), into the genome of YL-15 to generate YL-23. The culture medium of YL-23 was either left untreated or exogenously supplemented with 0.5% glucuronic acid and 2% myo-inositol (MI). The production of QA-C3-GGX has been analyzed by LC-MS (using respective standards).

### Results

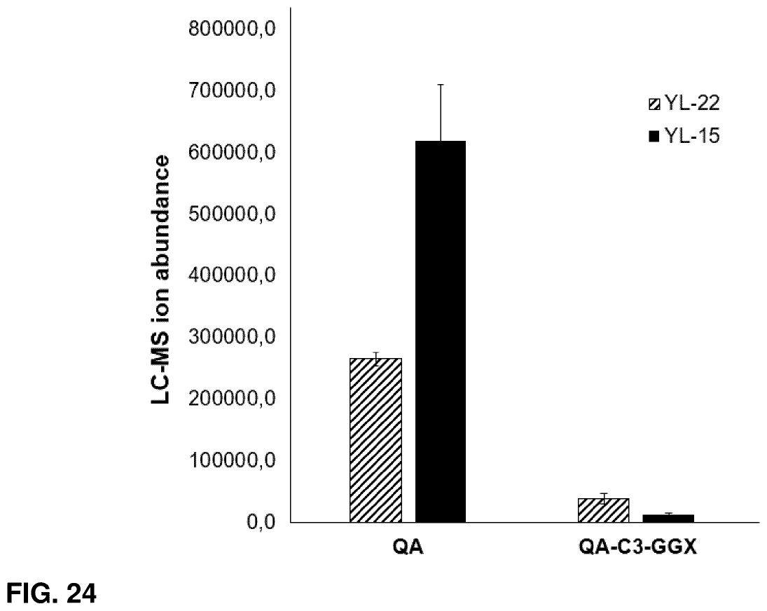

QA-C3-GGX production was improved by 3-fold in YL-22, as the residual QA decreased significantly (see FIG. 24).

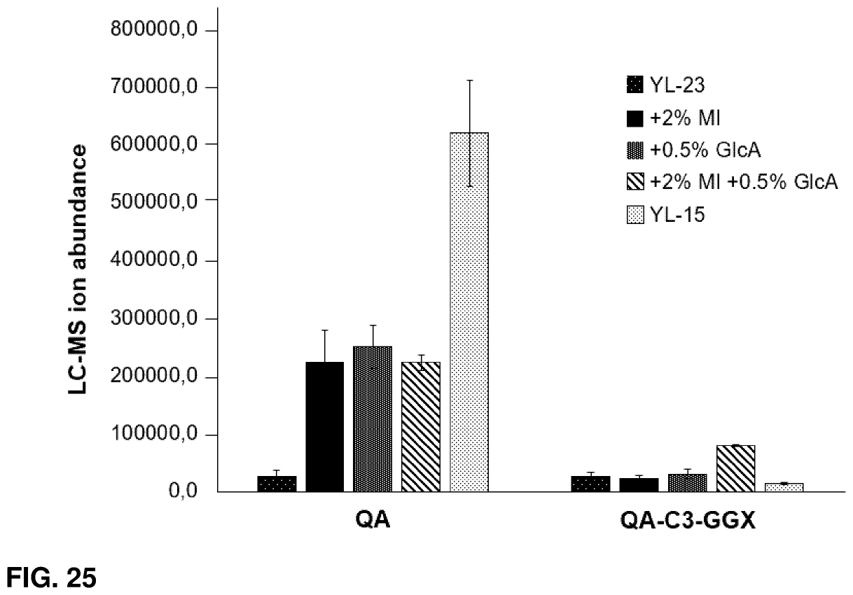

QA-C3-GGX production, in YL-23 (further overexpressing TtMIOX), was increased by 1.7-fold and 2.3-fold, in the presence of 2% MI and 0.5% GlcA exogenously supplemented, respectively. Production was further improved by 5.9-fold when both MI and GlcA were supplemented (see FIG. 25).

### Inducible TetOn Promoter to Delay the Expression of UXS to Accumulate UDP-GlcA

Inducible promoters such as pDDI2 (induced by methyl methane sulfonate), pCup1 (induced by copper ions), as well as pTetOn (induced by tetracycline or doxycycline) have been investigated and used, as a way to delay the expression of AtUXS. AtUXS has been overexpressed in a yeast engineered to produce QA-C3-GGX under a pTetOn promoter. Production of QA, QA-C3-GG and QA-C3-GGX has been analyzed by LC-MS.

### Results

pTetOn was compatible with the galactose promoters used in the parent yeast strain and the protein expression of AtUXS was linearly dependent on the concentration of the inducer.

In the absence of any inducer, a 5.5-fold increase of QA-C3-GGX production was observed, possibly because of the basal level expression of AtUXS due to the leakiness of the promoter. The minimal amount of UDP-Xyl produced may not be sufficient to inhibit AtUGD.

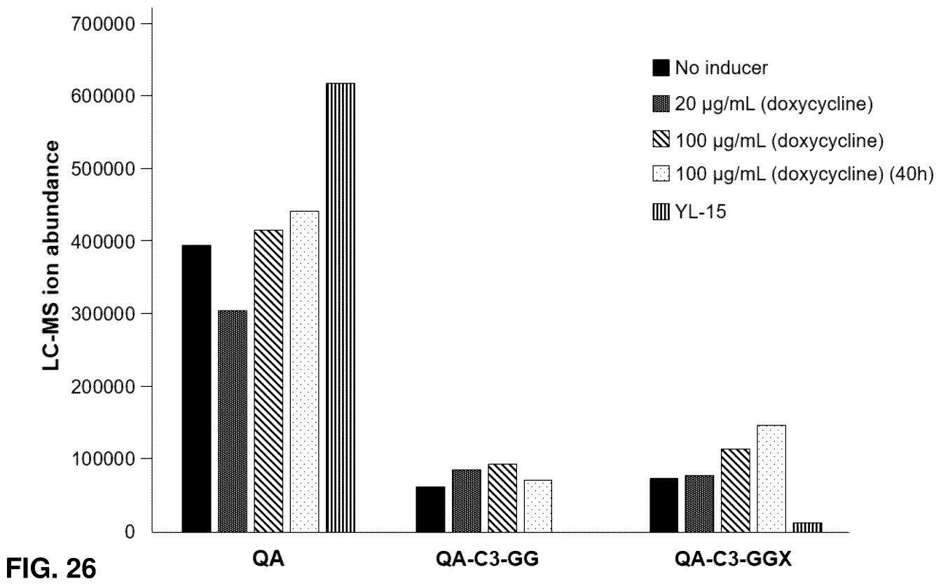

In order to induce pTetOn, 20 or 100 μg/mL of doxycycline has been added exogenously supplemented in the yeast culture medium 24 h after galactose induction. This led to the increased production of QA-C3-GGX by 5.9- and 8.5-fold, as compared to YL-15. When induced with 100 μg/mL of doxycycline after 40 h after galactose induction, an 11-fold increase was observed (see FIG. 26).

Identification of a C3XylT Enzyme in S. vaccaria

The inventors also identified in the transcriptome of S. vaccaria a novel gene encoding a xylosyl transferase candidate (named ‘SvC3XylT’—SEQ ID NO: 100 encoded by SEQ ID NO: 101). The function of SvC3XylT as a xylose transferase has been tested by transiently co-expressing the same SvCslG enzyme and SvGalT enzyme as described earlier in N. benthamiana plants. Plants have been infiltrated with 40 μM of QA (commercially available, e.g. from MedChemExpress) 2 days after Agrobacterium tumefaciens infiltration. QA-C3-GlcA-Gal-Xyl production has been analyzed by LC-MS. A standard corresponding to ‘QA-GlcpA-Galp-Xylp’ generated as described in WO 20/260475 has been used as a reference.

### Results

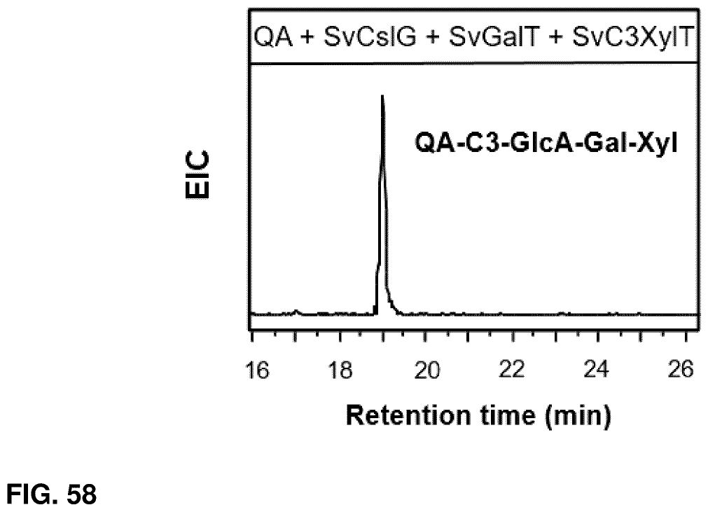

FIG. 58 shows that a peak corresponding to QA-C3-GlcA-Gal-Xyl was observed when co-expressing SvCslG, SvGalT and SvC3XylT, demonstrating the ability of SvXylT to transfer UDP-Xyl to QA-GlcA-Gal, confirming thus its functional relevance and activity.

### Conclusion

Using different heterologous enzymes (glycosyl synthases, glycosyl transferases) from different plant origins (e.g. A. thaliana, Q. saponaria and S. vaccaria), in different combinations, the inventors have been able to reconstruct in yeast the metabolic pathway leading to the biosynthesis of C3-glycosylated QA derivatives, achieving, for the first time, the successful production of such C3-glycosylated QA derivatives in yeast.

### Example 3—C28-Glycosylated QA Derivatives Biosynthesis

### Production of Fucose Non-Native to Yeast

The transcriptome of S. vaccaria was further explored to identify genes and enzymes involved in saponin biosynthesis, as S. vaccaria contains a number of different saponins that have similarity to saponins in Q. saponaria. S. vaccaria plants were treated with methyl-jasmonate (Meja) which was shown to induce biosynthesis of saponins in plants. An extensive RNASeq analysis was then performed to identify the full-length transcripts in the plants, and to identify the induced genes. Among them, several genes were known to be involved in biosynthesis of the triterpene backbone (e.g. β-amyrin synthase), as well as several Cytochrome P450 enzymes (CYP) and glycosyltransferase genes (see e.g. WO 20/263524). Some of the genes are homologs to genes known to be involved in saponin biosynthesis in Q. Saponaria (see e.g. WO 19/122259, WO 20/260475, WO 22/136563; Decker and Kleczkowski 2017). Based on knowledge from dTDP-D-Fucose biosynthesis in bacteria and UDP-L-Rhamnose biosynthesis in plants, it was predicted the pathway to include a dehydratase step and a reductase step (as shown in FIG. 12). No homologs of the enzymes involved in biosynthesis of dTDP-D-Fucose were found in bacteria. A homolog of a Q. saponaria UDP-4-keto-6-deoxy-glucose reductase gene was discovered in the S. vaccaria transcriptome, which was named ‘svNMD’. A candidate UDP-glucose-4,6-dehydratase that was induced by methyl-jasmonate and belongs to the family of nucleotide sugar epimerases was also discovered. The predicted enzyme, named ‘svUG46DH’, has similarity to a domain of UDP-L-Rhamnose synthase in plants. It was hypothesized that the two enzymes, sv46DH and svNMD, would catalyze the conversion of UDP-D-glucose to UDP-D-fucose (see FIG. 12). The functional relevance and activity of these newly identified genes has been tested in yeast, assessing for their ability to produce UDP-Fucose, in combination with the following enzymes:

svUG46DH (SEQ ID NO: 87 encoded by SEQ ID NO: 89) and svNMD (SEQ ID NO: 90 encoded by SEQ ID NO: 92) have both been integrated into the genome of the parent yeast strain CEN.PK2-1c to generate SC-19.

SvUG46DH and SvNMD have both been integrated into the genome of SC-4 (overexpressing AtUGD-AtUXS) to generate SC-20.

SvUG46DH and SvNMD have both been integrated into the genome of SC-17 (overexpressing AtRHM2) to generate SC-22.

SvUG46DH and SvNMD have both been integrated into the genome of SC-18 (overexpressing AtUGD-AtUXS-AtRHM2) to generate SC-23.

A homolog reductase from Q. saponaria (WO 22/136563) (named ‘QsFucSyn’—SEQ ID NO: 175 encoded by SEQ ID NO: 177) has been alternatively tested, in combination with the following enzymes:

QsFucSyn and SvUG46DH have been integrated into the genome of SC-4 (overexpressing AtUGD-AtUXS) to generate SC-21.

The production of UDP-Fucose has been analyzed by LC-MS.

### Results

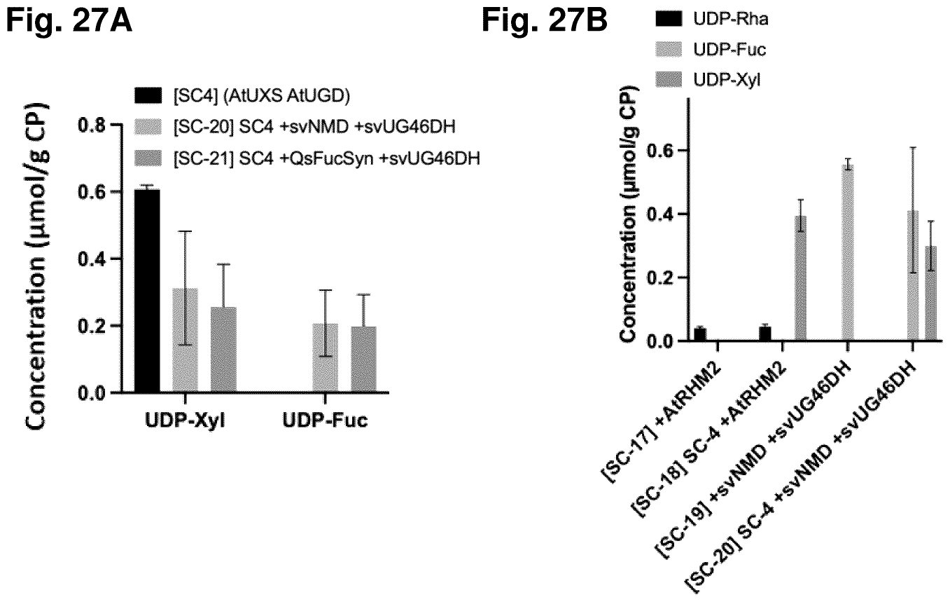

UDP-Fucose was produced when svUG46DH and svNMD were overexpressed on their own (SC-19) (see FIG. 14A and FIG. 14B or FIG. 27A and FIG. 27B).

UDP-Fucose was also produced when svUG46DH and svNMD were overexpressed together with AtUGD-AtUXS (SC-20) (see FIG. 14A and FIG. 14B or FIG. 27A and FIG. 27B).

UDP-Fucose was also produced when svUG46DH and svNMD were overexpressed together with AtRHM2 (SC-22) (see FIG. 14A and FIG. 14B).

UDP-Fucose was also produced when svUG46DH and svNMD were overexpressed together with AtUGD-AtUXS-AtRHM2 (SC-23) (see FIG. 14A and FIG. 14B).

UDP-Fucose was also produced when QsFucSyn and svUG46DH were overexpressed together with AtUGD-AtUXS (SC-21) (see FIG. 14A and FIG. 14B or FIG. 27A).

These results confirm the functional relevance and activity of the newly identified SvUG46DH and SvNMD, and QsFucSyn, when expressed in yeast.

### Production of QA-C3-GlcA-Gal-Rha/Xyl-C28-Fuc

A fucose transferase from Q. saponaria (WO 22/136563) (named ‘QsFucT’—SEQ ID NO: 93 encoded by SEQ ID NO: 95) has been integrated into the genome of YL-14 to generate YL-25.

### Results

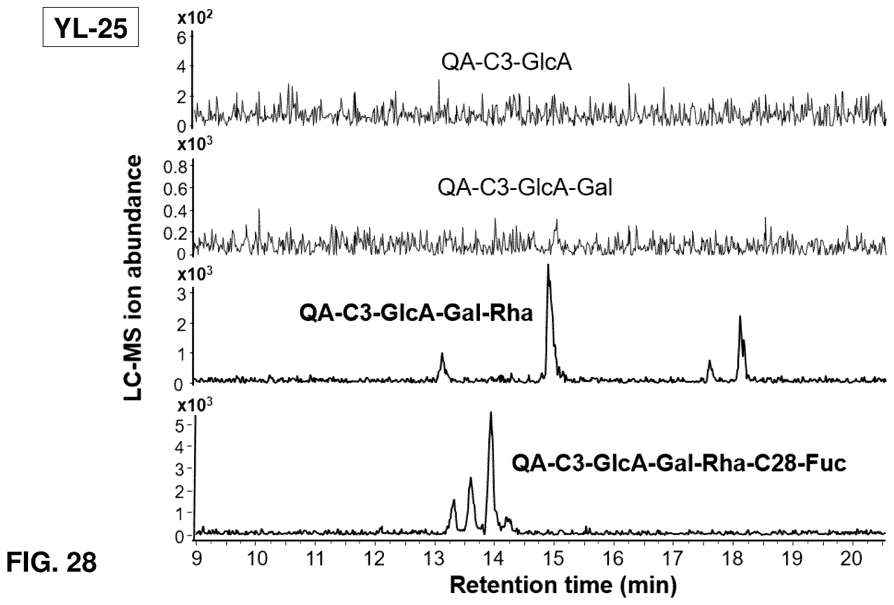

QA-C3-GlcA-Gal-Rha and QA-C3-GlcA-Gal-Rha-C28-Fuc have been detected in YL-25 (see FIG. 28), confirmed by co-eluting with the respective standards (QA-C3-GlcA-Gal-Rha standard corresponds to ‘QA-TriR’, generated as described in WO 22/136563 and QA-C3-GlcA-Gal-Rha-C28-Fuc standard corresponds to ‘QA-TriR-F’, also generated as described in WO 22/136563).

The same QsFucT enzyme has also been integrated into the genome of YL-15 to generate YL-26. QA-C3-GlcA-Gal-Rha-C28-Fuc production has been analyzed by LC-MS.

### Results

Production of QA-C3-GlcA-Gal-Xyl-C28-Fuc has been similarly observed in YL-26.

Identification of a FucT Enzyme in S. vaccaria

The inventors also identified in the transcriptome of S. vaccaria a novel gene encoding a FucT candidate (named ‘SvFucT’—SEQ ID NO: 96 encoded by SEQ ID NO: 97). The function of SvFucT as a fucose transferase has been tested by transiently co-expressing the same SvCslG, SvUG46DH and SvNMD as described earlier in N. benthamiana plants. Plants have been infiltrated with 40 μM of QA (commercially available, e.g. from MedChemExpress) 2 days after Agrobacterium tumefaciens infiltration. QsFucT (see above) was used as a positive control, and GFP was used as negative control. The production of QA-C3-GlcA-C28-Fuc has been analyzed by LC-MS.

### Results

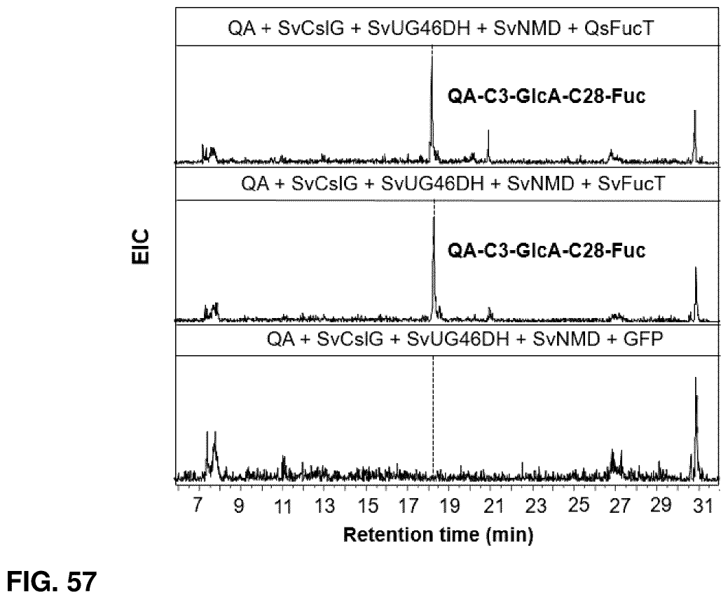

FIG. 57 shows that, when overexpressing SvFucT, a peak was observed at the same retention time (see the dashed line), as when overexpressing QsFucT (positive control), demonstrating the ability of SvFucT to transfer UDP-Fuc to QA-GlcA, confirming thus the functional relevance and activity of the newly identified SvFucT.

### Production of QA-C3-GlcA-Gal—Xyl-C28-Fuc-Rha

The same trifunctional AtRHM2 enzyme as described earlier, together with a rhamnose transferase from Q. Saponaria (named ‘QsRhaT’—SEQ ID NO: 119 encoded by SEQ ID NO: 121), has been integrated into the genome of YL-15 to generate YL-28. QA-C3-GlcA-Gal-Xyl-C28-Fuc-Rha production has been analyzed by LC-MS (using a standard which has been chemically synthesized as a reference).

### Results

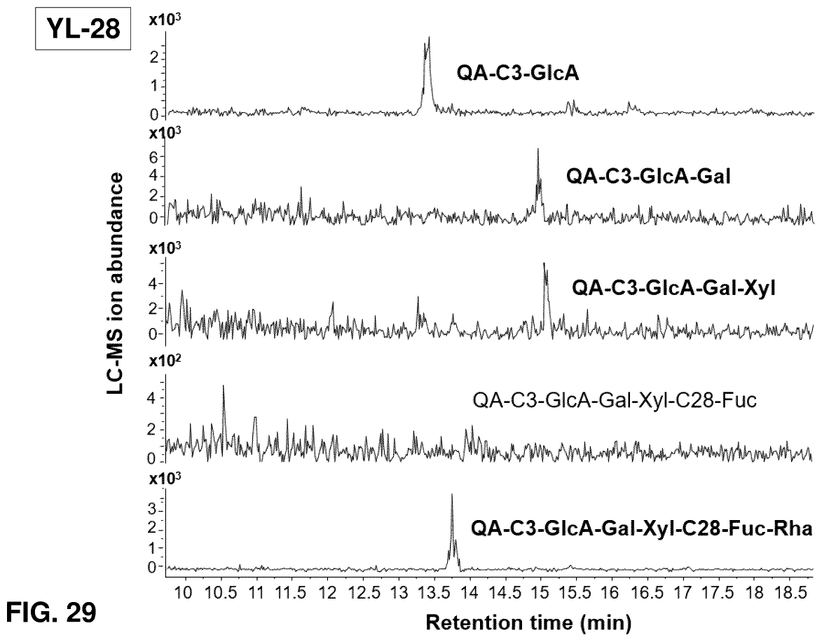

QA-C3-GlcA-Gal-Xyl-C28-Fuc-Rha production was detected in YL-28 at a titer of about 1 mg/L (see FIG. 29). No residual substrate was observed, indicating that QsRhaT is highly efficient and catalyzes the complete conversion of QA-C3-GlcA-Gal-Xyl-C28-Fuc to QA-C3-GlcA-Gal-Xyl-C28-Fuc-Rha.

### Production of QA-C3-GlcA-Gal-Rha-C28-Fuc-Rha

An additional copy of the same trifunctional AtRHM2 enzyme (as described earlier), together with the same QsRhaT (as described earlier) has been integrated into the genome of YL-14 to generate YL-27. QA-C3-GlcA-Gal-Rha-C28-Fuc-Rha production has been analyzed by LC-MS. A standard corresponding to ‘QA-TriR-FR’ as described in WO 22/136563 has been used as a reference.

### Results

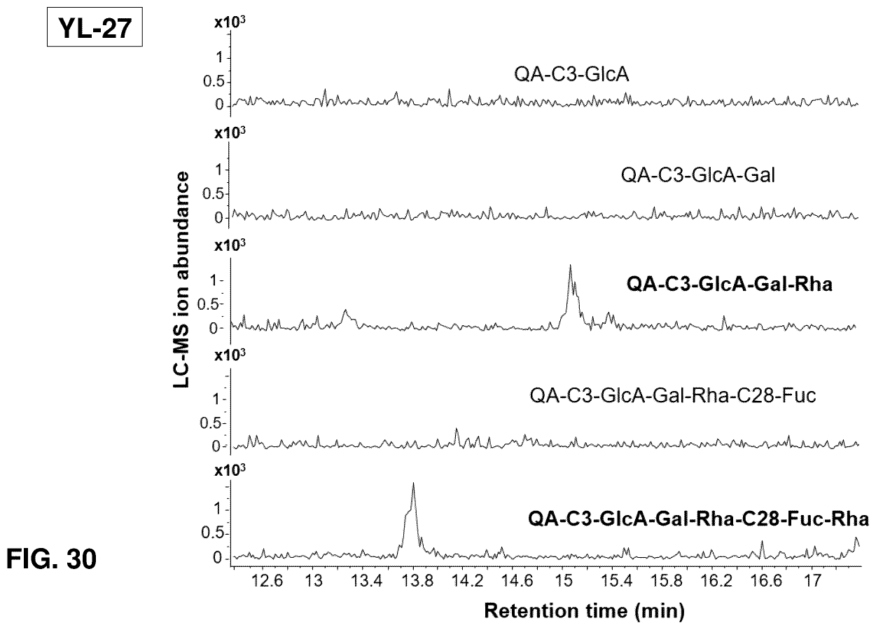

QA-C3-GlcA-Gal-Rha-C28-Fuc-Rha was detected in YL-27 at a titer of about 3 mg/L (see FIG. 30). No residual substrate was observed, indicating that QsRhaT is highly efficient and catalyzes the complete conversion of QA-C3-GlcA-Gal-Rha-C28-Fuc to QA-C3-GlcA-Gal-Rha-C28-Fuc-Rha.

### Production of QA-C3-GlcA-Gal-Xyl-C28-Fuc-Rha-Xyl

An additional copy of the same AtUXS enzyme (as described earlier), together with a xylose transferase from Q. Saponaria (named ‘QsC28XylT3’—SEQ ID NO: 125 encoded by SEQ ID NO: 127), has been integrated into the genome of YL-28 to generate YL-30. QA-C3-GlcA-Gal-Xyl-C28-Fuc-Rha-Xyl production has been analyzed by LC-MS. QA-C3-GGR-C28-FRX previously obtained was used a reference.

### Results

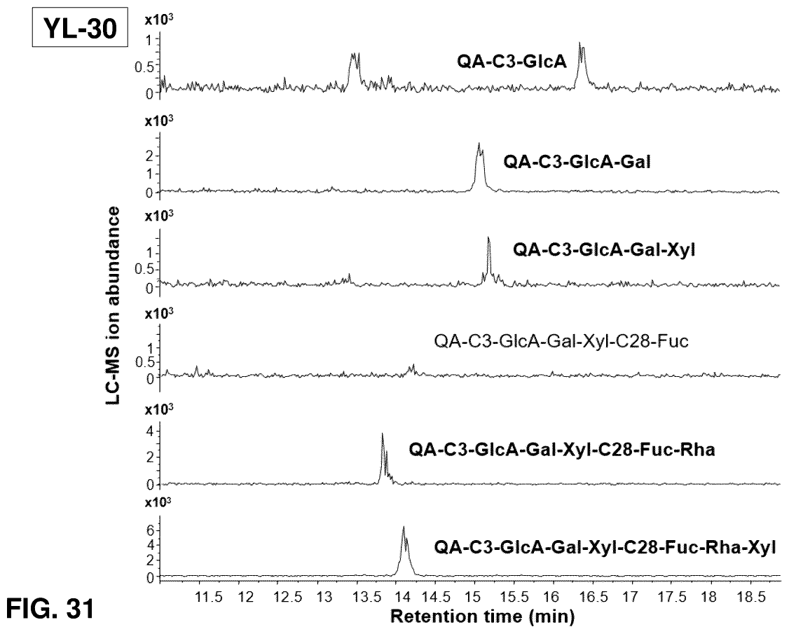

QA-C3-GlcA-Gal-Xyl-C28-Fuc-Rha-Xyl was detected in YL-30 (see FIG. 31).

### Production of QA-C3-GlcA-Gal-Rha-C28-Fuc-Rha-Xyl

The same AtUXS enzyme (as described earlier), together with the same QsC28XylT3 as above, was integrated into the genome of YL-27 to generate YL-29. The production of QA-C3-GlcA-Gal-Rha-C28-Fuc-Rha-Xyl has been analyzed by LC-MS. A standard corresponding to ‘QA-TriR-FRX’ as described in WO 22/136563 has been used as a reference.

### Results

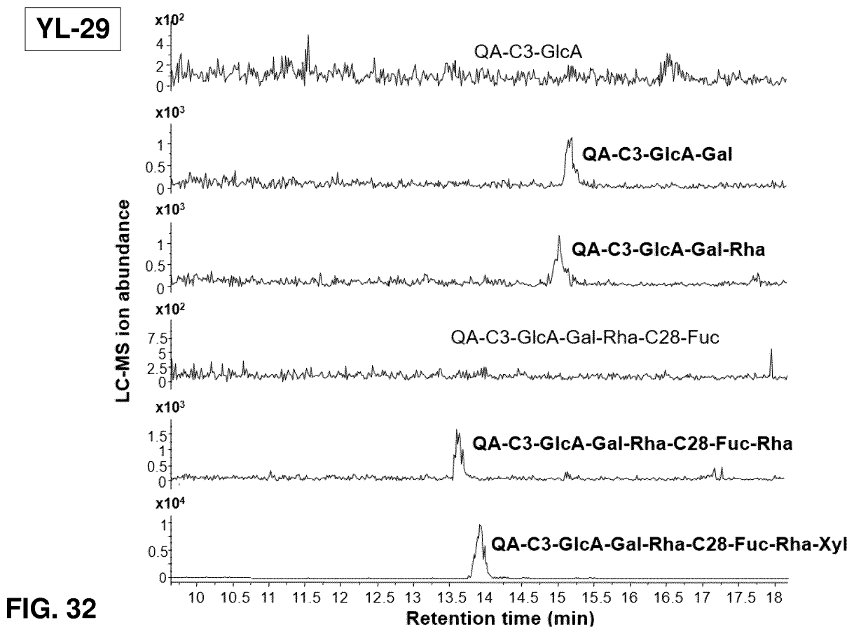

QA-C3-GlcA-Gal-Rha-C28-Fuc-Rha-Xyl was detected in YL-29 (see FIG. 32).

### Production of QA-C3-GlcA-Gal-Xyl-C28-Fuc-Rha-Xyl-Xyl

An additional copy of the same AtUXS enzyme (as described earlier), together with a xylose transferase from Q. Saponaria (named ‘QsC28XylT4’—SEQ ID NO: 128 encoded by SEQ ID NO: 130), has been integrated into the genome of YL-30 to generate YL-33. The production of QA-C3-GlcA-Gal-Xyl-C28-Fuc-Rha-Xyl-Xyl has been analyzed by LC-MS. QA-C3-GGR-C28-FRXX previously obtained was used a reference.

### Results

Conversion of QA-C3-GGX-C28-FRX into QA-C3-GGX-C28-FRX was observed in YL-33 (FIG. 33).

### Production of QA-C3-GlcA-Gal-Rha-C28-Fuc-Rha-Xyl-Xyl

An additional copy of the same AtUXS enzyme (as described earlier), together with the same C28QsXylT4 as above, has been integrated into the genome of YL-29 to generate YL-31. The production of QA-C3-GlcA-Gal-Rha-C28-Fuc-Rha-Xyl-Xyl has been analyzed by LC-MS. A standard corresponding to ‘QA-TriR-FRXX’ as described in WO 22/136563 has been used as a reference.

### Results

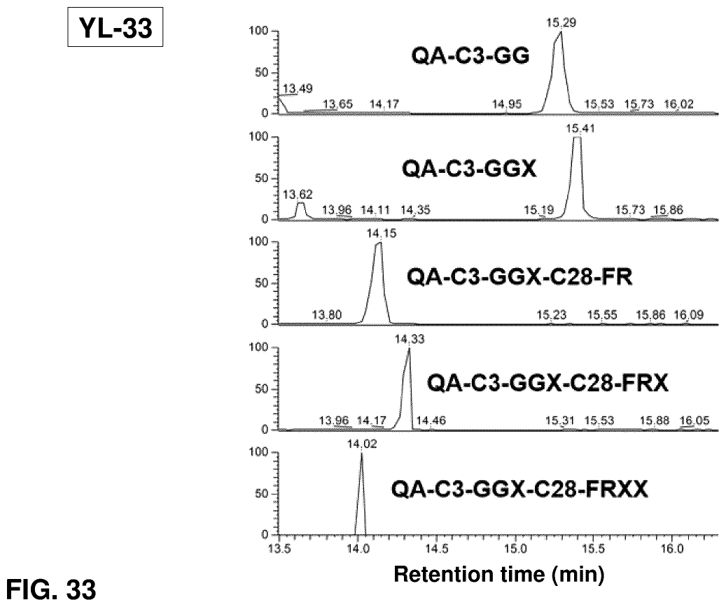

Conversion of QA-C3-GlcA-Gal-Rha-C28-Fuc-Rha-Xyl into QA-C3-GlcA-Gal-Rha-C28-Fuc-Rha-Xyl-Xyl was observed in YL-31 (FIG. 34).

### Production of Apiose Non-Native to Yeast

UDP-Apiose can be produced using apiose synthase (‘AXS’) enzymes, which produces both UDP-Xyl and UDP-Api (as shown in FIG. 12). The same AtUGD enzyme as above (as described earlier) and an apiose synthase from Q. saponaria (named ‘QsAXS'-SEQ ID NO: 113 encoded by SEQ ID NO: 115) have been integrated into the genome of the parent yeast strain CEN.PK2-1c to generate SC-16. UDP-Apiose is a very unstable compound with a half-life of 100 min at room temperature. While UDP-Apiose was not detectable (data not shown), it is likely it was produced but degraded during the extraction process, and was thus undetectable via LC-MS.

### Production of QA-C3-GcA-Gal-Xyl-C28-Fuc-Rha-Xyl-Api

An additional copy of the same QsAXS enzyme as above, together with an apiose transferase from Q. Saponaria (named ‘QsC28ApiT4’—SEQ ID NO: 151 encoded by SEQ ID NO: 153), has been integrated into the genome of YL-30 to generate YL-34. The production of QA-C3-GlcA-Gal-Xyl-C28-Fuc-Rha-Xyl-Api has been analyzed the by LC-MS.

### Results

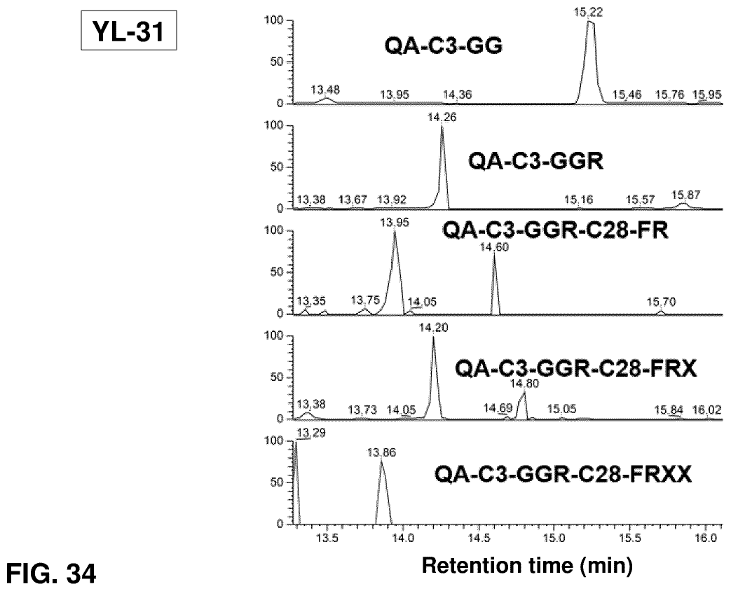

Conversion of QA-C3-GlcA-Gal-Xyl-C28-Fuc-Rha-Xyl into QA-C3-GlcA-Gal-Xyl-C28-Fuc-Rha-Xyl-Api was observed in YL-34 (FIG. 35).

### Production of QA-C3-GlcA-Gal-Rha-C28-Fuc-Rha-Xyl-Api

The same QsAXS enzyme as above, together with the same QsC28ApiT4 enzyme as above, has been integrated into the genome of YL-29 to generate YL-32. The production of QA-C3-GlcA-Gal-Rha-C28-Fuc-Rha-Xyl-Api has been analyzed the by LC-MS.

### Results

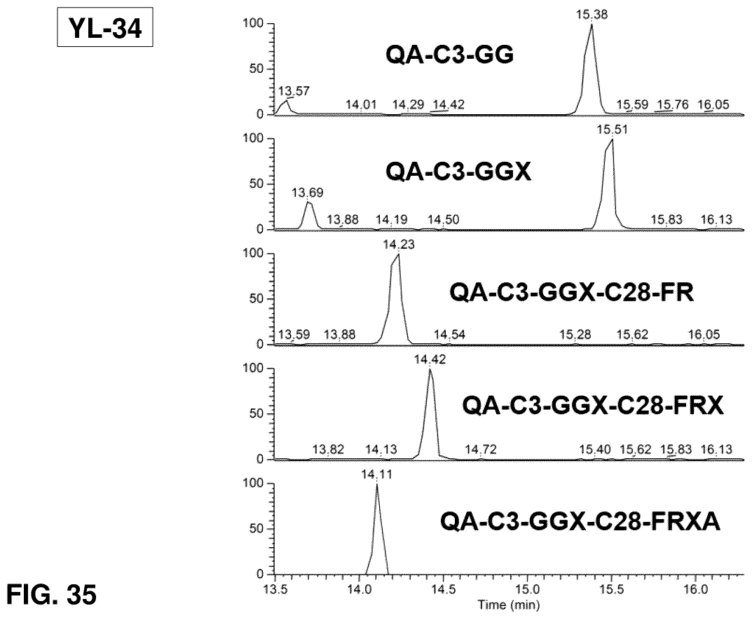

Conversion of QA-C3-GlcA-Gal-Rha-C28-Fuc-Rha-Xyl to QA-C3-GlcA-Gal-Rha-C28-Fuc-Rha-Xyl-Api was observed in YL-32 (FIG. 36).

### Conclusion

Using different heterologous enzymes (glycosyl synthases and glycosyl transferases) from different plant origins (e.g. A. thaliana, Q. saponaria and S. vaccaria), the inventors have been able to reconstruct in yeast the metabolic pathway leading to the synthesis of C28-glycoslylated QA derivatives, achieving, for the first time, the successful production of such C28-glycoslylated QA derivatives in yeast.

Different approaches have been investigated to assess whether the conversion of QA-C3-GGR/X-C28-FRX into QA-C3-GGR/X-FRXX/A could be improved.

Subcellular localization of QsC28XylT4 and QsC28ApiT4

The subcellular localization of QsC28XylT4 and QsC28ApiT4 heterologously expressed in yeast was examined. C-terminal green fluorescent protein (GFP) fusion was built to provide QsC28XylT4-GFP and QsC28ApiT4-GFP in order to visualize the subcellular localization in yeast (using QsC28XylT3-GFP as a reference). Each of QsC28XylT4-GFP, QsC28ApiT4-GFP, and QsC28XylT3-GFP has been integrated into the genome of the parent yeast strain CEN.PK2-1c.

### Results

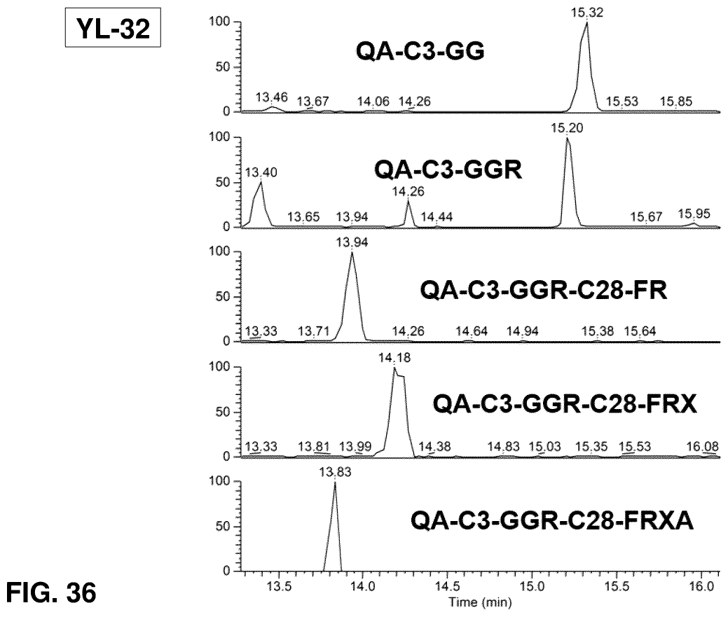

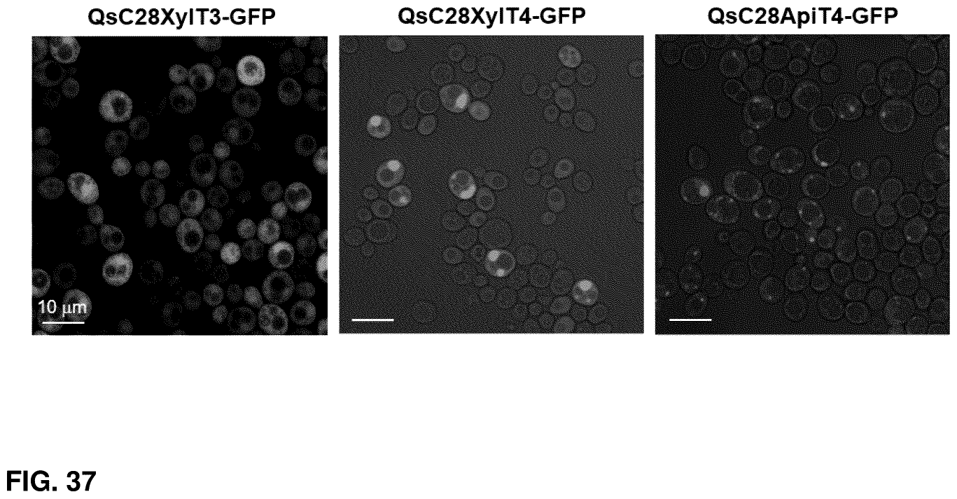

While flow cytometry data (aimed at measuring the absolute protein expression level) showed similar fluorescence intensity, indicating a similar level of protein expression (data not shown), confocal microscopy images revealed that, unlike QsC28XylT3-GFP (FIG. 37) which shows a cytosolic localization, QsC28XylT4-GFP (FIG. 37) and QsC28ApiT4-GFP (FIG. 37) formed aggregates, generally known as inclusion bodies in yeast.

### Identification of the Localization of QsXylT4 and QsApiT4 Aggregation

Three signature localization protein markers have been selected to identify the subcellular localization where QsC28XylT4 and QsC28ApiT4 aggregates are formed with the aim to functionally express the two enzymes in the cytosol. Rnq1 which is a yeast native prion protein has been shown to co-localize with ‘insoluble protein deposit’ (‘IPOD’), a reservoir and degradation location for amyloid-like proteins. C-terminal mcherry-tagged Rnq1 was expressed in yeast independently to visualize IPOD, shown to be a perivascular compartment (data not shown). The co-expression of Rnq1-mcherry with QsC28XylT4-GFP revealed a different localization pattern, suggesting that QsC28XylT4 is likely not an amyloid-like misfolded protein (data not shown). The second protein marker, heat shock protein-42 (Hsp-42), has been selected due to its suggested physiological role in initiation of stress granules in yeast upon starvation in carbon or nitrogen sources. Hsp42-mcherry fusion protein was localized in the cytosol and nucleus of yeast (data not shown) and was shown to be co-localized with QsXylT4-GFP (data not shown), suggesting the possible sequestration of QsC28XylT4 into stress granules. The last protein marker selected was Rpn1, a functional component of the proteasome actively involved in the protein degradation machinery. When expressed alone, Rpn1, together with the proteasome machinery, was localized in the nucleus. Upon co-expression with QsC28XylT4-GFP, while the majority of Rpn1-mcherry still remained in the nucleus at 24 h (data not shown), it formed aggregates around QsC28XylT4-GFP aggregates at 48 h and degraded the aggregates towards protein recycling (data not shown). These results suggest that QsC28XylT4 may be sequestered into Hsp42-related stress granule and be prone to degradation.

### N-Terminal Truncation or Solubility Tagging of QsC28XylT4

Truncation of the N-terminus of QsC28XylT4, with the increment of three amino acids up to 12, as well as addition of solubility tags, such as SUMO, TrXA, and MBP, have been carried as an attempt to re-direct the protein in the cytosol. ‘QsC28XylT4-3aa′ (QsC28XylT4 deleted from the 3 first amino acids—SEQ ID NO: 131 encoded by SEQ ID NO: 133), ‘QsC28XylT4-6aa′ (QsC28XylT4 deleted from the 6 first amino acids—SEQ ID NO: 134 encoded by SEQ ID NO: 136), ‘QsC28XylT4-9aa′ (QsXylT4 deleted from the 9 first amino acids—SEQ ID NO: 137 encoded by SEQ ID NO: 139), ‘QsC28XylT4-12 aa′ (QsC28XylT4 deleted from the 12 first amino acids—SEQ ID NO: 140 encoded by SEQ ID NO: 142), ‘SUMO-QsC28XylT4’—SEQ ID NO: 143 encoded by SEQ ID NO: 144, ‘TrXA-QsC28XylT4’—SEQ ID NO: 145 encoded by SEQ ID NO:146 and ‘MBP-QsC28XylT4’—SEQ ID NO: 147 encoded by SEQ ID NO:148 have each been integrated into the genome of YL-30 to generate YL-35, YL-36, YL-37, YL-38, YL-39 and YL-40, respectively. The level of QsC28XylT4 protein expression in each yeast strain and the ability of each yeast strain to produce QA-C3-GlcA-Gal-Xyl-C28-Fuc-Rha-Xyl-Xyl (as compared with YL-33, harboring a wild-type, full-length, non-tagged, QsXylT4) have been looked at.

### Results

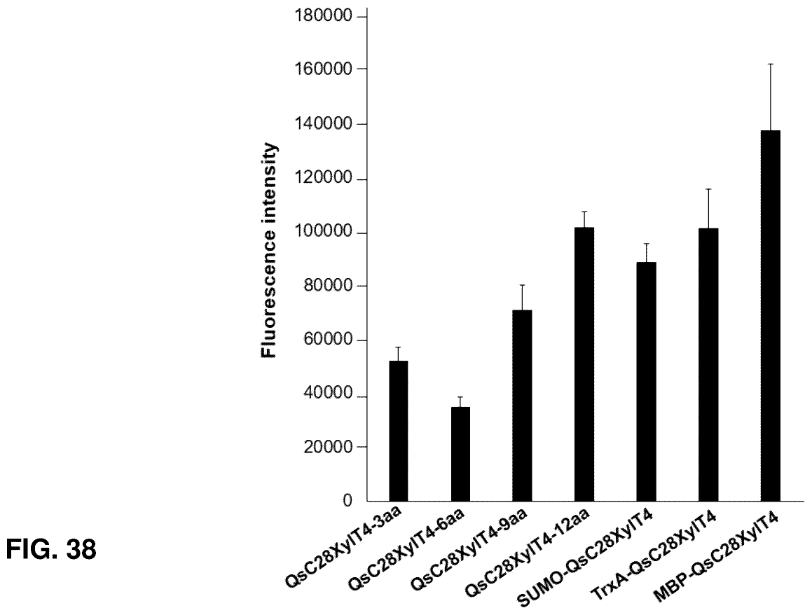

The fluorescence intensity measured by flow cytometry shows the highest level of protein expression for QsC28XylT4-MBP (see FIG. 38).

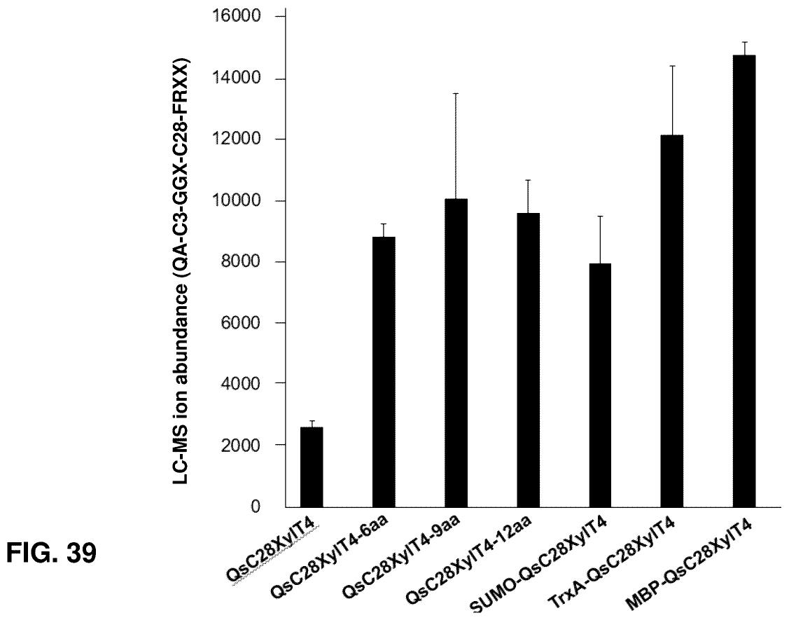

In terms of production of QA-C3-GlcA-Gal-Xyl-C28-Fuc-Rha-Xyl-Xyl, all N-terminal truncations of QsC28XylT4 and all N-terminal-tagged QsC28XylT4 showed a better yield, with the N-terminus MBP tag addition providing a 7-fold increase, as compared with the wild-type and full-length of the enzyme (see FIG. 39).

### N-Terminal Fusion of QsXylT3 to QsXylT4

As an alternative way to render QsC28XylT4 cytosolic, QsC28XylT3 (shown to be cytosolic when expressed in yeast, as described earlier), was fused at the N-terminus of QsC28XylT4. A 3×GGGS linker was genetically inserted between the two amino acid sequences of the enzymes to ensure the flexibility of the linker and independent folding of the two enzymes, without affecting the functional properties of the fusion protein. The fusion QsC28XylT3-3×GGGS-QsC28XylT4 (SEQ ID NO: 149 encoded by SEQ ID NO: 150) has been integrated into the genome of YL-30 to generate YL-41. The localization of the fusion protein and the production of QA-C3-GlcA-Gal-Xyl-C28-Fuc-Rha-Xyl-Xyl have been looked at.

### Results

Confocal microscopy images showed an improved cytosolic expression with less level of aggregation observed for the QsXylT3-3×GGGS-QsXylT4-GFP fusion protein, as compared to QsXylT4-GFP when expressed alone (see FIG. 40).

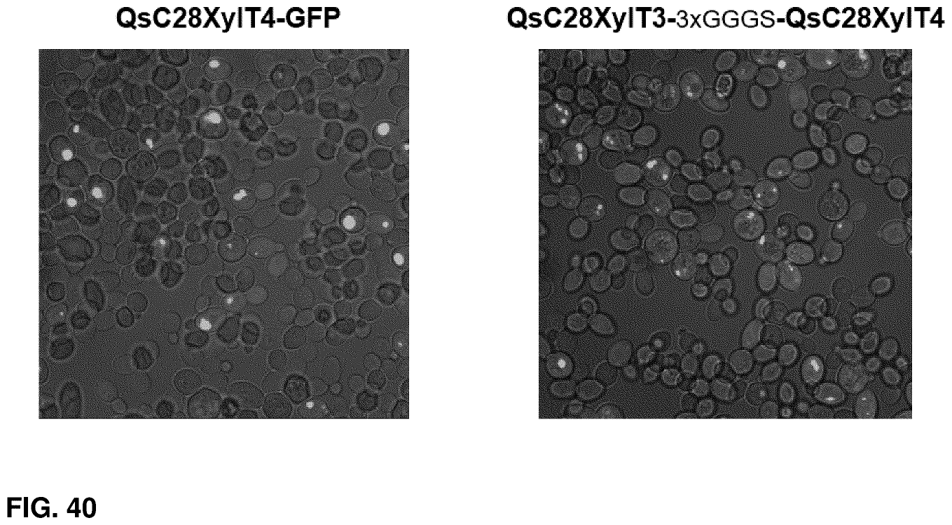

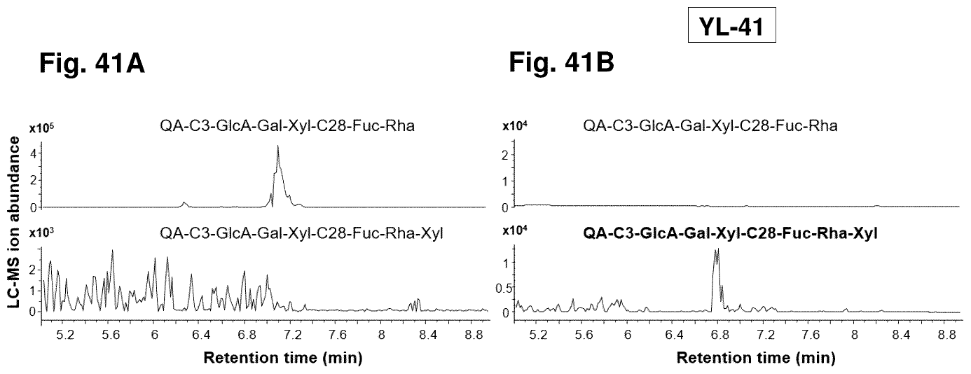

The improved reactivity of the fusion protein was also confirmed by the observation of the complete conversion of QA-C3-GlcA-Gal-Xyl-C28-Fuc-Rha-Xyl which leads to a distinctive peak corresponding to the mass of QA-C3-GlcA-Gal-Xyl-C28-Fuc-Rha-Xyl-Xyl (see FIG. 41B), compared to the absence of the peak in the yeast strain where QsXylT3 and QsXylT4 were expressed separately (see FIG. 41A).

### Continuous Feeding Scheme to Decrease QsXylT4 Sequestration and Degradation

A continuous feeding scheme has been devised by adding fresh nitrogen and/or carbon sources every 24 h. Protein expression and protein localization have been looked at.

### Results

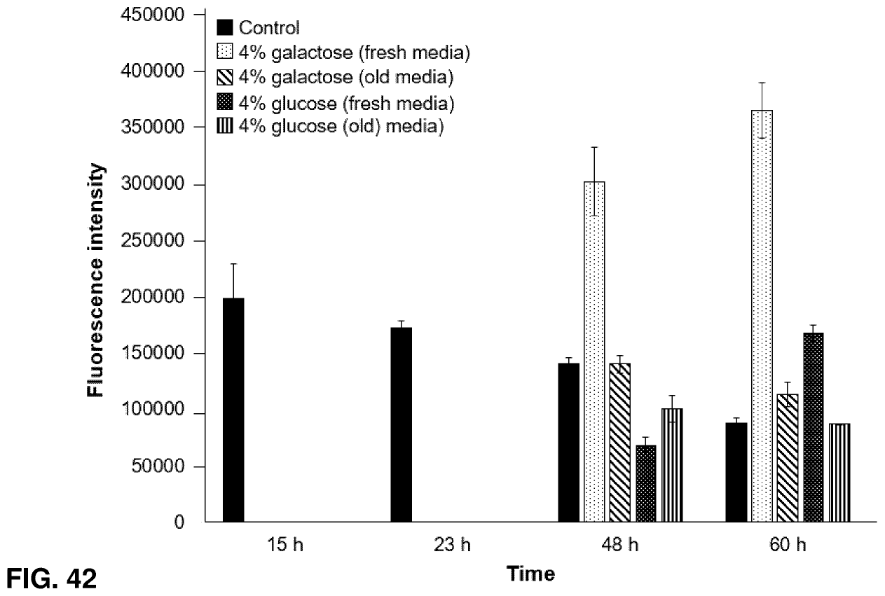

The fluorescence intensity, reflecting QsC28XylT4 absolute expression, has been measured by flow cytometry (FIG. 42). The protein expression decreased over the course of 60 h of the experiment, while spiking additional carbon source only (glucose or galactose) had no impact on the expression level.

In contrast, the addition of fresh media with additional nitrogen source as well as 4% galactose consistently increased QsC28XylT4 expression level up to 5-fold by 60 h.

In the presence of new carbon and nitrogen sources, QsXylT4 did not colocalize with Hsp42. Rpn1, which represents the localization of the proteasome machinery, remained in the nucleus and did not degrade QsXylT4 aggregates. While some aggregation of QsXylT4 persisted in the presence of new media, consistent cytosolic expression was also observed, in stark contrast to yeast strains cultured with only old media, where QsXylT4 expression in the cytosol was depleted after 24 h (data not shown).

### Example 4—18-Carbon Pseudo-Dimeric Acyl Chain Terminated with Araf Biosynthesis

As shown in FIG. 2A, acylation requires the biosynthesis and two consecutive additions of C9-CoA; two chalcone-synthase-like type III polyketide synthases (PKSs) stitch two malonyl CoA and one unit of (S)-2-methylbutyryl-CoA (2 MB-CoA) to form the C9-keto-CoA which is subsequently reduced by two standalone keto-reductases (KRs) to yield the C9-CoA.

4.1 (S)-2-Methylbutyryl CoA (2 MB-CoA) Conversion from the 2 MB Acid

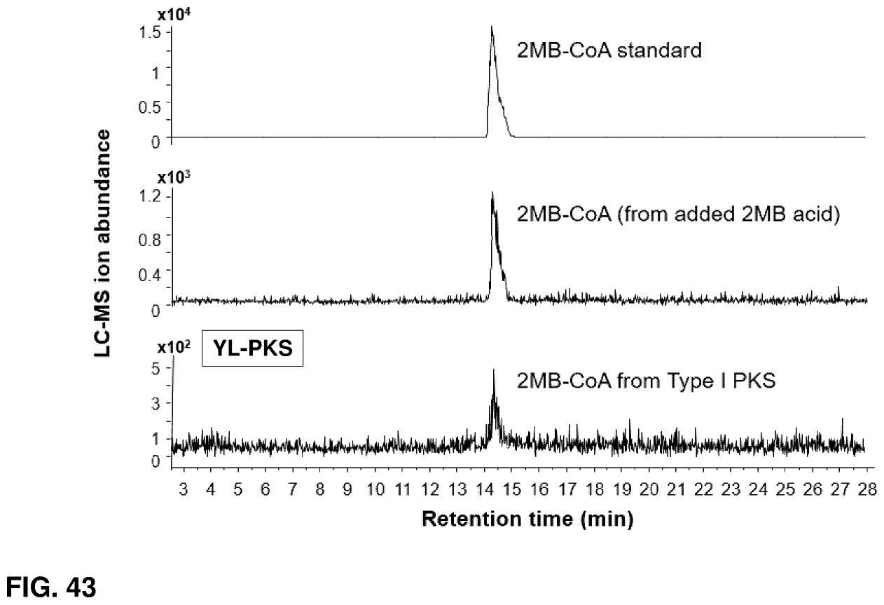

Conversion of exogenously supplemented 2 MB acid to 2 MB-CoA by a CoA ligase identified from Q. Saponaria transcriptome has been investigated. The functional expression of this CoA ligase from Q. Saponaria (named ‘QsCCL’—SEQ ID NO: 178 encoded by SEQ ID NO: 180) has been confirmed using a high-copy plasmid transfected into the parent yeast strain CEN.pk2-1c via confocal microscopy imaging of the C-terminal GFP fusion of the enzyme, which is visualized to be in the cytoplasm and is stable for at least 24 h after galactose induction (data not shown). Additionally, the conversion of 2 MB acid to 2 MB-CoA by QsCCL has been demonstrated using a whole-cell feed-in experiment. 2 MB acid has been added directly to the yeast cell culture and the yeast cells have been lysed to allow the measurement of the intracellular content of 2 MB-CoA, by a liquid chromatography method using a porous graphitic carbon column. Production of 2 MB-CoA from 50 mg/L 2 MB acid in YL-QsCCL has been confirmed by co-eluting with a 2 MB-CoA standard (the standard has been chemically synthesized) (see FIG. 43).

### 4.2 Biosynthesis and Reduction of Keto-C9-CoA to Make C9-CoA

As shown in FIG. 2A and FIG. 2D, the 18-carbon acyl chain consists of two repeating C9 units. They are synthesized from two units of malonyl CoA and one 2 MB-CoA using two chalcone-synthase-like type III PKSs (named ‘QsChSD’—SEQ ID NO: 181 encoded by SEQ ID NO: 183 and ‘QsChSE’—SEQ ID NO: 184 encoded by SEQ ID NO: 186); the product C9-Keto-CoA is then reduced by two keto-reductases (named ‘QsKR11’—SEQ ID NO: 187 encoded by SEQ ID NO: 189 and ‘QsKR23’—SEQ ID NO: 190 encoded by SEQ ID NO: 192) to form C9-CoA. The functional expression of these four enzymes has been confirmed using high-copy plasmids in yeast via confocal microscopy imaging of the C-terminal GFP fusion of the corresponding enzymes. The expression of both QsChSD and QsChSE is shown to be cytosolic, with a low degree of aggregation in the case of QsChSE. While the expression of KR11 is shown to be in the cytoplasm, the expression of QsKR23 is shown to be localized to the endoreticulum (ER) membrane (data not shown).

### 4.3 Addition of C9-CoAs to C3- and C28-Glycosylated QA Derivatives

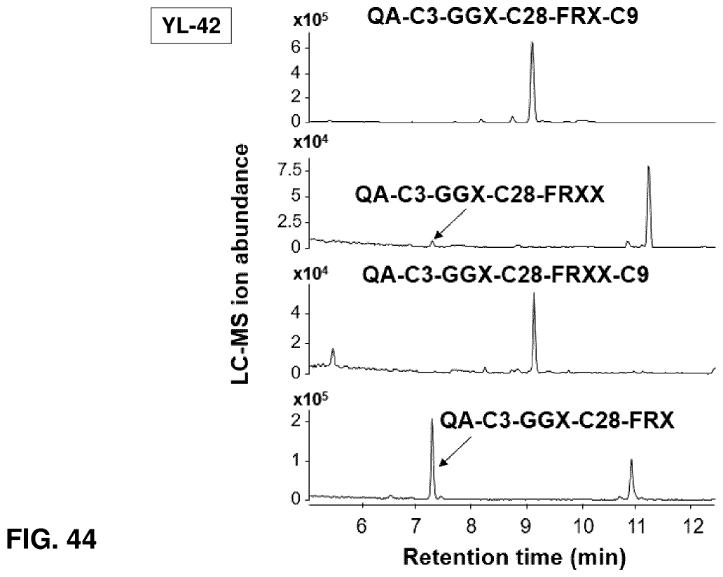

Attempts to directly detect the production of C9-CoA as such using LC-MS was unsuccessful, possibly due to its short-lived stability. Therefore, the synthesis of C9-CoA was demonstrated by its addition to glycosylated QA derivatives. It has been demonstrated that the acyl unit (C9-CoA) can be added to both QA-C3-GlcA-Gal-Xyl-C28-Fuc-Rha-Xyl (QA-C3-GGX-C28-FRX) and QA-C3-GlcA-Gal-Xyl-C28-Fuc-Rha-Xyl-Xyl (QA-C3-GGX-C28-FRXX) (FIG. 44). Because of the higher LC-MS response of QA-C3-GGX-C28-FRX, the yeast strain producing such glycosylated QA derivative (YL-30) was used to harbor the genomic integration of a large cassette containing both QsChsD and QsChSE, both QsKR11 and QsKR23, QsCCL, and an acyl transferase (named ‘QsDMOT9’—SEQ ID NO: 193 encoded by nucleotide sequence SEQ ID NO: 195) to generate YL-42. Production of QA-C3-GGX-C28-FRX-C9 has been analysed by LC-MS.

### Results

YL-42 is shown to produce QA-C3-GGX-C28-FRX-C9 in the presence of 50 mg/L 2 MB acid added exogenously, as confirmed by co-eluting with a standard (standard has been generated in N. benthamiana, as described in GB 2204252.7) (see FIG. 44).

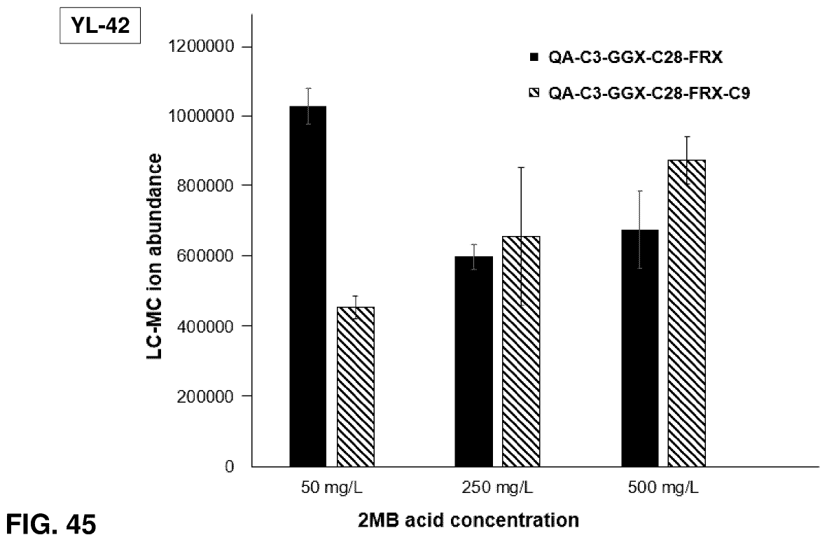

The conversion of QA-C3-GGX-C28-FRX to QA-C3-GGX-C28-FRX-C9 was improved in the presence of a higher concentration of 2 MB acid supplemented to the growth media (FIG. 45).

The 18-carbon acyl chain consists of two repeating units of C9-CoA, and the second addition requires its corresponding acyltransferase (named ‘QsDMOT4’—SEQ ID NO: 196 encoded by nucleotide sequence SEQ ID NO: 198), which has been integrated into the genome of YL-42 to generate YL-43.

### Results

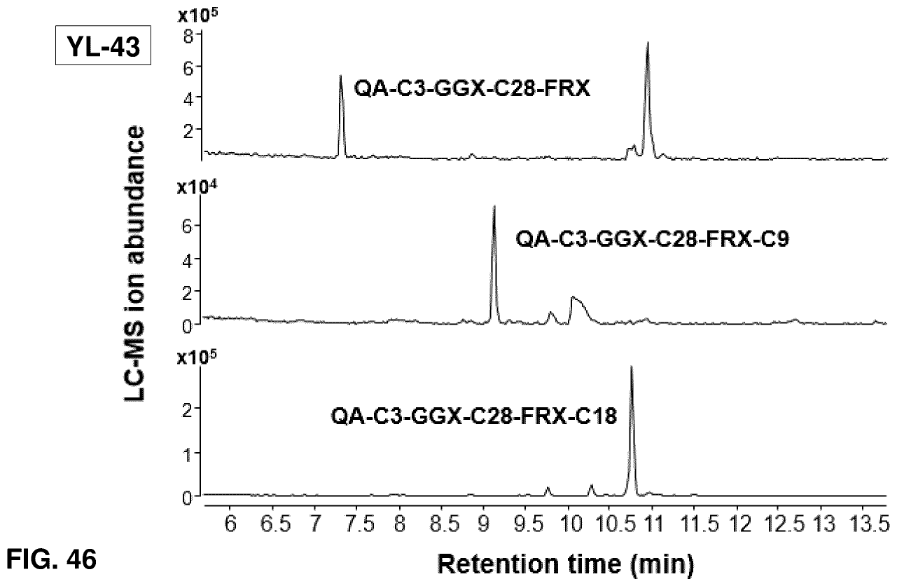

With 500 mg/L 2 MB acid supplemented to the culture media, the production of QA-C3-GGX-C28-FRX-C18 has been confirmed with the appearance of a new LC-MS peak with the same high-resolution mass and its conversion from QA-C3-GGX-C28-FRX-C9 was shown to be highly efficient with little residual substrate (FIG. 46), suggesting that 2 MB acid supplement and endogenous malonyl CoA pool provide sufficient C9-CoA for two acyl additions.

In order to generate QA-C3-GGX-C28-FRXX-C18, QsDMTO4 and QsC28XylT4 have been integrated into the genome of YL-42 to generate YL-44. QA-C3-GGX-C28-FRXX-C18 production has been analyzed by LC-MS.

### Results

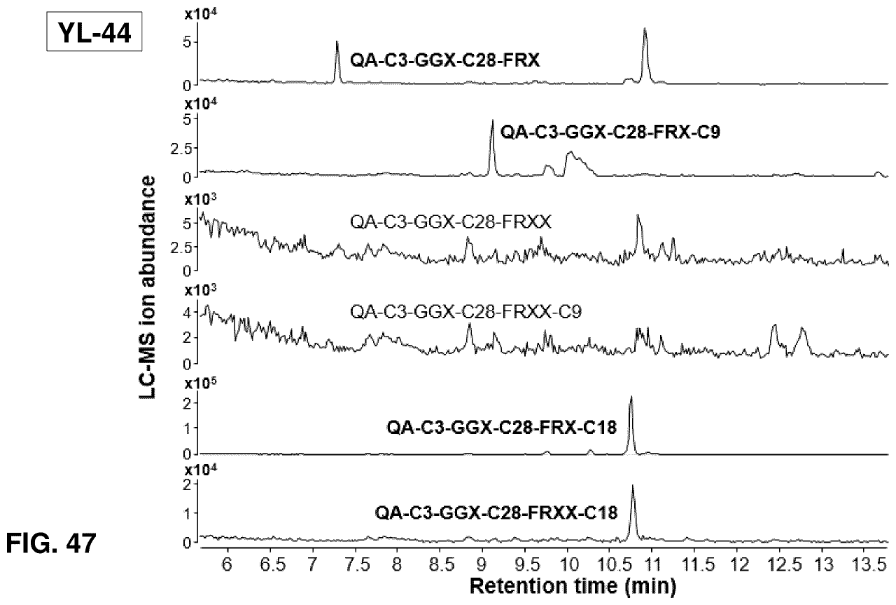

A new LC-MS peak corresponding to the mass of QA-C3-GGX-C28-FRXX-C18 was detected (FIG. 47).

The absence of QA-C3-GGX-C28-FRXX and QA-C3-GGX-C28-FRXX-C9 suggests that they are better substrates for QsDMOT9 and QSDMOT4 acyltransferases than QA-C3-GGX-C28-FRX and QA-C3-GGX-C28-FRX-C9.

4.4 Production of UDP-Arabinofuranose (Araf) Non-Native in Yeast

The biosynthesis of UDP-Araf is not native in yeast and thus, necessary nucleotide sugar synthases as well as an arabinosyl transferase, are required for the heterologous production and addition of this sugar. As shown in FIG. 12, UDP-Xyl can be first converted to UDP-Arabinopyranose via a UDP-Xyl epimerase (UXE), which then undergoes ring-chain tautomerization assisted by UDP-Arabinose mutases (UAMs). UAM from A. thaliana (‘AtUAM1′—according to SEQ ID NO: 208 encoded by the nucleotide sequence SEQ ID NO: 210), H. vulgare (‘HvUAM’—according to SEQ ID NO: 211 encoded by the nucleotide sequence SEQ ID NO: 213), UXE from A. thaliana (‘AtUXE’—according to SEQ ID NO: 199 encoded by the nucleotide sequence SEQ ID NO: 201), ‘AtUXE2′ (according to SEQ ID NO: 202 encoded by the nucleotide sequence SEQ ID NO: 204) and/or ‘AtUGE3′ (according to SEQ ID NO: 205 encoded by the nucleotide sequence SEQ ID NO: 207) have been integrated into SC-4, according to the following combinations:
AtUAM1-AtUXE2 (SC-9)
HvUAM-AtUXE (SC-10)
AtUAM1-AtUGE3 (SC-11)
Sugar production has been analyzed by LC-MS.

### Results

UDP-Xyl was produced by all combinations of enzymes, with AtUAM1-AtUGE3 (SC-11) producing lower UDP-Xyl.

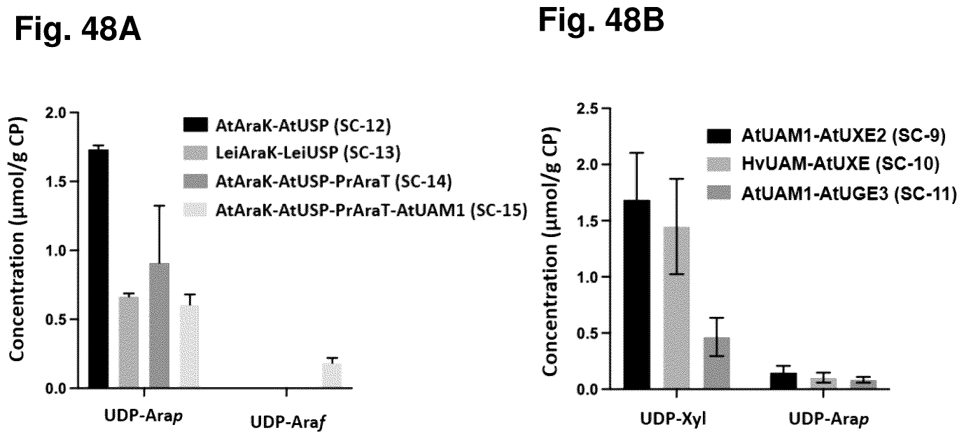

While UDP-Arap production was similar, UDP-Araf was not detected, likely due to the co-elution with UDP-Xyl and since both UXE and UAM enzymes are dominated by equilibrium, UDP-Araf is likely 100× less in abundance than UDP-Xyl (see FIG. 48B).

As an alternative, the salvage pathway has been tested with arabinokinase (AraK) and UDP-sugar pyrophosphorylase (USP) candidates from A. thaliana (named ‘AtAraK’-SEQ ID NO: 214 encoded by nucleotide sequence SEQ ID NO: 216 and AtUSP—SEQ ID NO: 223 encoded by nucleotide sequence SEQ ID NO: 225, respectively) and Leptospira interrogans (Lei) (named ‘LeiAraK’—SEQ ID NO: 217 encoded by nucleotide sequence SEQ ID NO: 219 and ‘LeiUSP’—SEQ ID NO: 226 encoded by nucleotide sequence SEQ ID NO: 228, respectively). An arabinose transporter from Penicillium rubens Wisconsin (named ‘PrAraT’—SEQ ID NO: 220 encoded by nucleotide sequence SEQ ID NO: 222) has also been tested to determine if it was necessary for arabinose to enter the yeast and AtUAM1 to convert UDP-Arap to UDP-Araf. The following combinations have been integrated into the genome of the parent yeast strain CEN.PK2-1c, wherein corresponding yeasts were fed with 1% arabinose added exogenously:
AtAraK-AtUSP (SC-12)
LeiAraK-LeiUSP (SC-13)
AtAraK-AtUSP-PrAraT (SC-14)
AtAraK-AtUSP-PrAraT-AtUAM1 (SC-15)

### Results

Both AraT and the salvage pathway from L. interrogans produced less UDP-Arap (0.910 μmol/g Cell Pellet and 0.665 μmol/g Cell Pellet, respectively), as compared to the salvage pathway from A. thaliana (1.73 μmol/g Cell Pellet).

UDP-Araf was produced with the salvage pathway, AraT and AtUAM1 at 0.185 μmol/g Cell Pellet (see FIG. 48A).

### 4.5 UDP-Arabinofuranose (Araf) Addition

Plant UDP-L-arabinofuranose (UDP-Araf) biosynthesis is closely associated with the golgi apparatus because L-Araf is a key component in the plant cell wall. The biosynthesis of UDP-Arap mainly occurs through the epimerization of UDP-Xyl in the Golgi lumen, which is interconverted into UDP-Araf by a UDP-Ara mutase located outside on the cytosolic surface of the Golgi, then being transported back to the Golgi lumen for its later glycosylation applications. Because of the lack of yeast native sugar transporters on the golgi membrane, cytosolic homologs of these enzymes were selected from A. thaliana, UDP-xylose epimerase (AtUXE) and AtUAM1 to produce UDP-Araf in yeast.

Starting from YL-42 (the yeast strain capable of producing QA-C3-GGX-C28-FRX-C9), genes encoding (i) AtUXS and QsC28XylT4 (to produce QA-C3-GGX-C28-FRXX-C9), or AtAXS and QsC28ApiT4 (to produce QA-C3-GGX-C28-FRXA-C9), (ii) QsDMOT4 (to produce QA-C3-GGX-C28-FRXX-C18 or QA-C3-GGX-C28-FRXA-C18), (iii) AtUXE and AtUAM1 (to produce arabinofuranose from UDP-Xyl), and (iv) an arabinofuranose transferase (named ‘QsArafT’—SEQ ID NO: 229 encoded by nucleotide sequence SEQ ID NO: 231) (to produce QA-C3-GGX-C28-FRXX-C18-A or QA-C3-GGX-C28-FRXA-C18-A) have been further integrated into the genome of YL-42, generating two new yeast strains, as summarized below, and 2 MB acid was supplemented in the culture media:
AtUXS-QsC28XylT4-AtUXE-AtUAM1-QsDMOT4-QsArafT (YL-45)
AtUXS-QsApiT4-AtUXE-AtUAM1-QsDMOT4-QsArafT (YL-46)

### Results

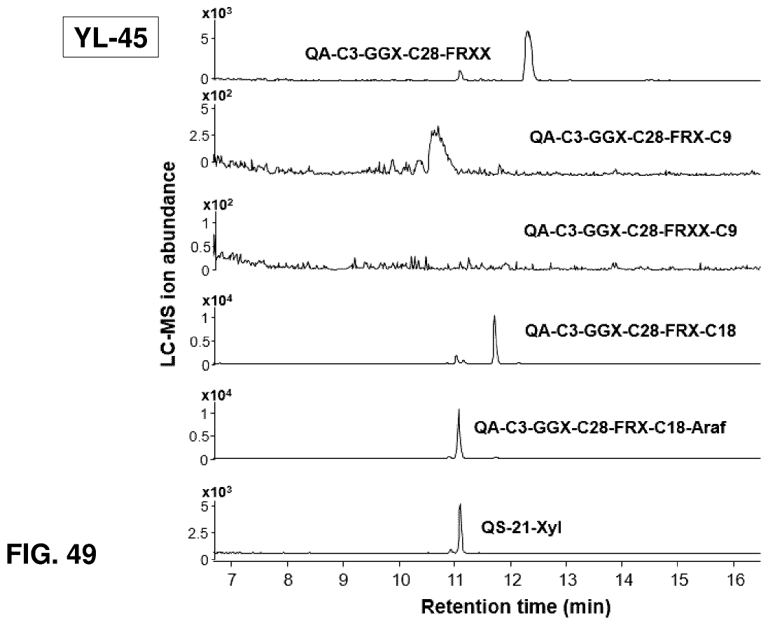

In the extracted single-ion LC-MS chromatogram of YL-45, more than one peak was observed when the exact mass of QS-21-Xyl (i.e. QA-C3-GGX-C28-FRXX-C18-A) was extracted (FIG. 49). Likewise, more than one peak was observed with the exact extracted mass of QA-C3-GGX-C28-FRXX-C18, which also corresponds to the mass of QA-C3-GGX-C28-FRX-C18-A.

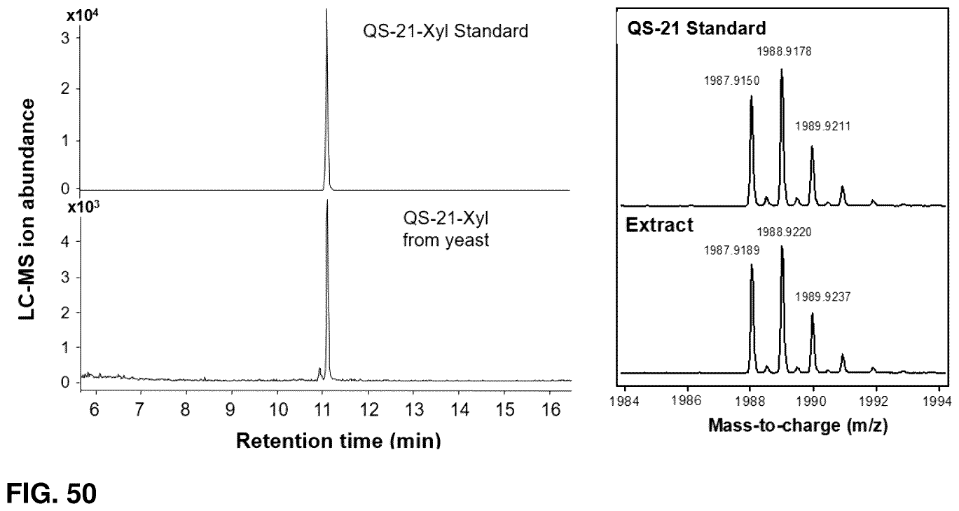

The peak with a retention time of 11.1-11.2 min co-elutes with a QS-21 standard (standard corresponds to the QS-21 fraction purified from a crude bark extract of Quillaja saponaria Molina which has been generated as described in WO 19/106192) (FIG. 50). The extracted sample was also spiked with the standard. The isotopic distribution of the peak extracted mass remained unchanged before and after the standard spiking (FIG. 50 inset), therefore confirming the production of QS-21-Xyl in YL-45 at a titer of 94.6 μg/L.

### Results

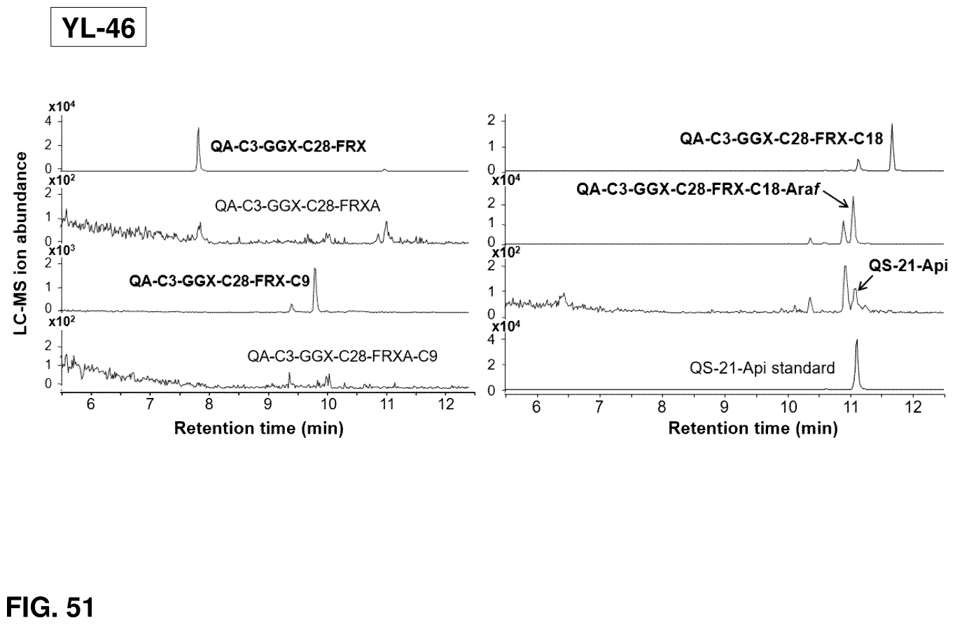

In the extracted single-ion LC-MS chromatogram of YL-46, multiple peaks are similarly observed when the exact mass of QS-21-Api (i.e. QA-C3-GGX-C28-FRXA-C18-A) was extracted (FIG. 51).

Additionally, similar peak patterns with the exact extracted mass of QA-C3-GGX-C28-FRXA-C18 are also observed, which also corresponds to the mass of QA-C3-GGX-C28-FRX-C18-A.

Since xylose, arabinofuranose (Araf), and arabinopyranose (Arap) are structural isomers, they also have the same exact mass. It is likely that other pentose sugars can be added instead of Araf, leading to the other peaks with the same exact mass as QS-21-Api. Therefore, the substrate scope of the Araf transferase (QsArafT) has been investigated. The strain YL-47 has been constructed by integrating QsArafT without the genes required to convert UDP-Xyl to UDP-Araf (i.e. without AtUXE and AtUAM1).

### Results

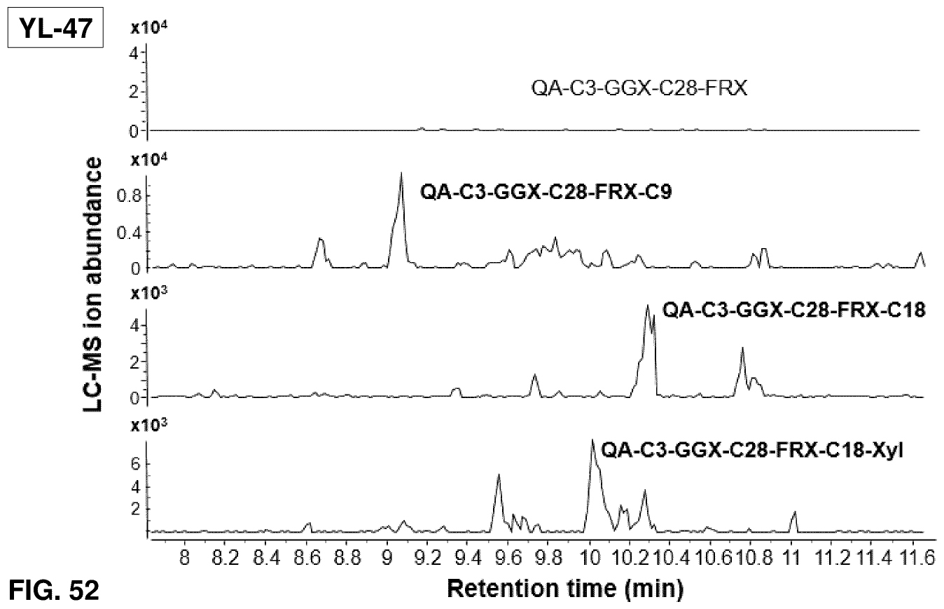

As a result, a new peak is observed that corresponds to QA-C3-GGX-C28-FRX-C18-Xyl, suggesting that QsArafT can also use UDP-Xyl as a substrate instead of UDP-Araf for addition at the end of the acyl chain (FIG. 52).

In search of ArafT homologs that are more specific towards UDP-Araf, BLAST searches were performed on the Q. saponaria transcriptome in 1 kp database (https://db.cngb.org/onekp/) using the ArafT protein sequence (SEQ ID NO: 229). A candidate with 64% protein homolog has been identified, OQHZ_scaffold_2012646, named ‘QsArafT2′ (SEQ ID NO: 232 encoded by the nucleotide sequence SEQ ID NO: 234). First, this candidate has been tested for its activity towards UDP-Xyl. YL-48 has been similarly constructed by integrating QsArafT2 without the genes required to convert UDP-Xyl to UDP-Araf (i.e. without AtUXE and AtUAM1), and 2 MB acid was supplemented in the culture media.

### Results

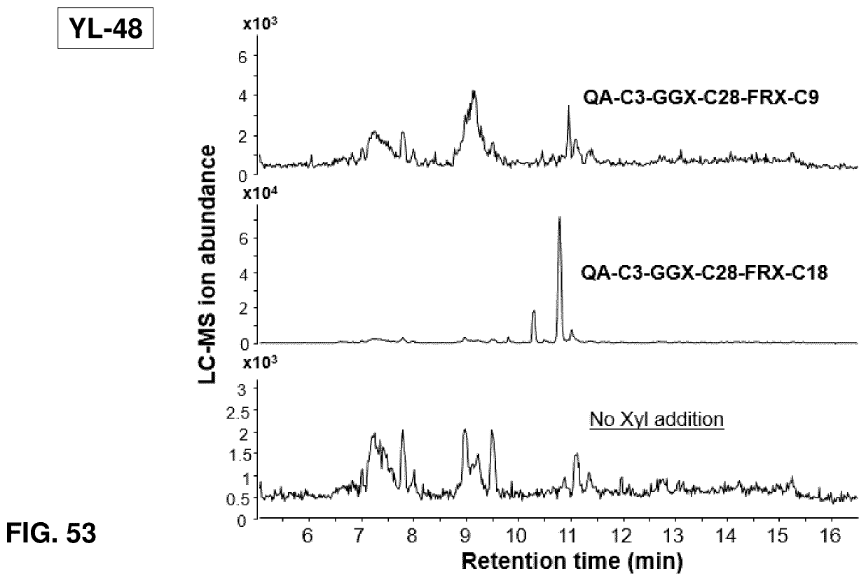

While the production of QA-C3-GXX-C18-FRX-C18 (in YL-48) has been detected, no LC-MS peak that corresponds to the addition of Xyl was observed (FIG. 53), suggesting that ArafT2 is not active towards using UDP-Xyl as a substrate.

Therefore, a new yeast strain was generated (YL-49), similar to YL-45, except that the gene encoding QsArafT was replaced with a gene encoding QsArafT2.

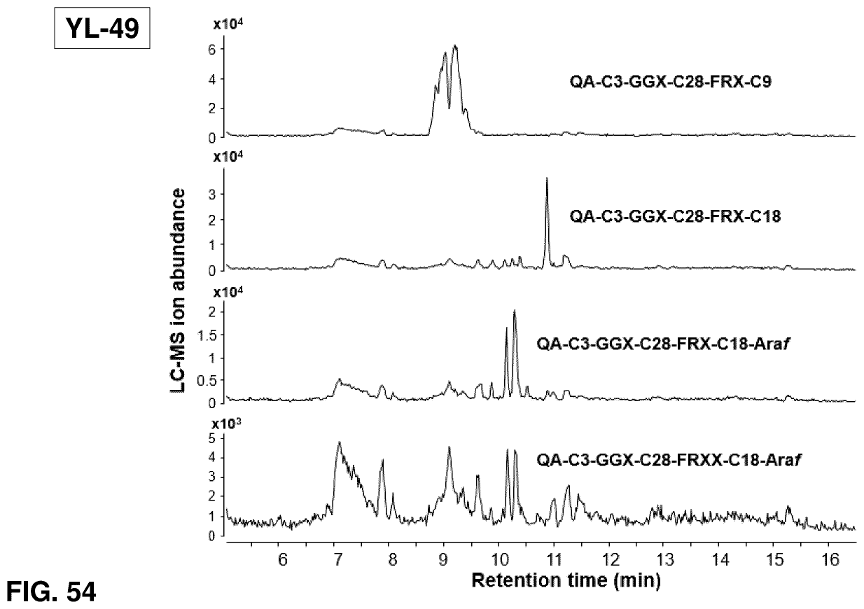

The extracted single-ion chromatograms confirmed the production of QS-21-Xyl (i.e. QA-C3-GXX-C18-FRXX-C18-Araf) with a higher ratio of the desired peak with regard to the other LC-MS peaks with the same exact mass (FIG. 54).

4.6 Integration of a Type I Polyketide Synthase to Produce (S)-2-Methylbutyryl CoA In Vivo

In order to circumvent the need of exogenously adding 2 MB acid, the biosynthesis of (S)-2-methylbutyryl CoA (2 MB-CoA) in vivo in yeast has been investigated. The branched-chain α-keto acid dehydrogenase (BCKD) complex has first been investigated with a transaminase from Bacillus subtilis (Bs), which, in bacteria, would readily convert isoleucine to 2 MB-CoA during amino acid metabolism. However, no 2 MB-CoA was detected in yeast engineered to express BsBKCD (data not shown). Without wishing to be bound to a theory, it is believed that this may be due to yeast lacking the necessary post-translational modification mechanism of the subunit E2 of the BKCD complex.

Alternatively, a 7.6 kb type I polyketide synthase (PKS) LovF from Aspergillus terreus (Ast) (also referred to as ‘Megasynthase LovF’) has been engineered to produce 2 MB-CoA in vivo. Native LovF condenses two units of malonyl-CoA to 2 MB-ACP, i.e. 2 MB covalently attached to the ACP (Acyl Carrier Protein) domain. In order to obtain free 2 MB, a promiscuous DEBS (6-deoxyerythronolide synthase) thioesterase (TE) domain from Saccharopolyspora erythraea (Se) has been fused at the C-terminus of LovF (also referred to as ‘LovF-TE’), to cleave 2 MB acid from the ACP domain. The resulting 2 MB acid can then be converted into 2 MB-CoA by QsCCL, similar to the case of 2 MB exogenous supplementation. An additional phosphopantetheinyl (Ppant) transferase is required for LovF to be functional in a heterologous host. Accordingly, a chromosomal copy of a Ppant candidate from Aspergillus nidulans (named ‘AnNpgA’ according to SEQ ID NO: 237 (encoded by the nucleotide sequence SEQ ID NO: 239) has been integrated into the genome of the parent yeast strain CEN.PK2-1c to generate YL-AnNpgA. A plasmid expressing AstLovF-TE according to SEQ ID NO: 235 (encoded by SEQ ID NO: 236) and QsCCL according to SEQ ID NO: 178 (encoded by SEQ ID NO: 180) has been transfected into YL-AnNpgA to generate YL-PKS. Additionally, AnNpgA and AstLovF-TE have been integrated into the genome of YL-42 (a yeast strain producing QA-C3-GGX-C28-FRX) to generate YL-42-AstLovF-TE, as well as into the genome of YL-45 (a yeast strain producing QA-C3-GGX-C28-FRXX-C18-Araf or QS-21-Xyl, in the presence of 2-MB supplemented exogenously) and YL-46 (a yeast strain producing QA-C3-GGX-C28-FRXA-C18-Araf or QS-21-Api, in the presence of 2-MB supplemented exogenously) to generate YL-50 and YL-51, respectively.

### Results

The production of 2 MB-CoA by YL-PKS (engineered with LovF-TE) has been confirmed by LC-MS (FIG. 43) demonstrating the successful type I PKS LovF-TE engineering that catalyzes the release of free 2 MB acid from ACP and subsequent CoA ligation by QsCCL.

While the peak integration of 2 MB-CoA is lower than that of the 2 MB acid feed-in experiment, the production of QA-C3-GGX-C28-FRX-C9 using NgpA and LovF-TE in YL-42-AstLovF-TE was more comparable with the feed-in experiment in the case of YL-42, approximately 50% (data not shown).

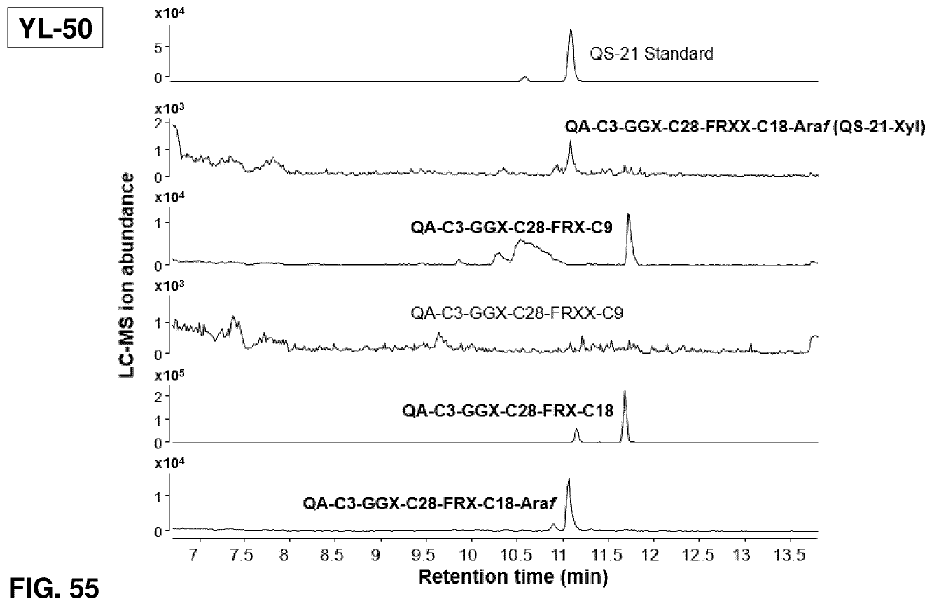

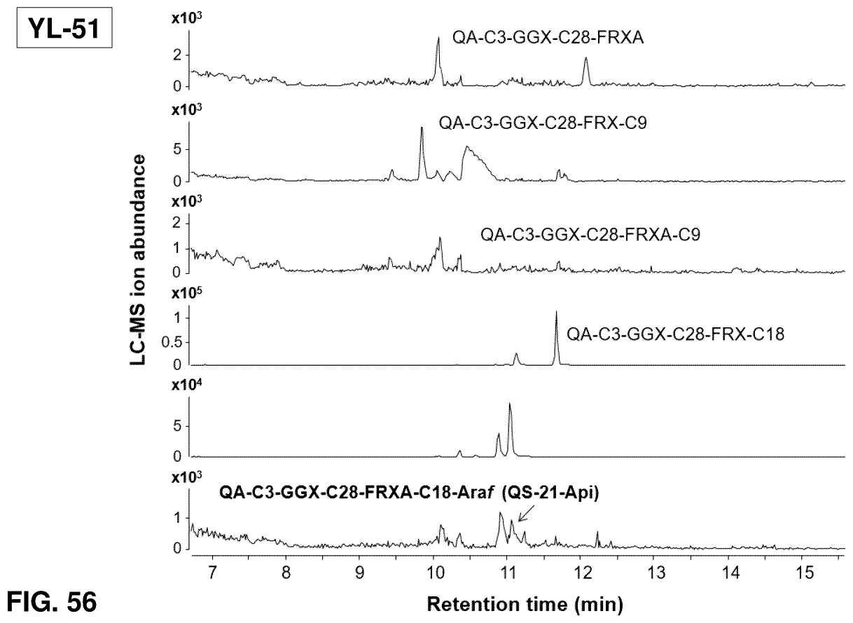

The complete biosynthesis of QS-21-Xyl and QS-21-Api in YL-50 and YL-51, respectively, was observed (FIG. 55 and FIG. 56, respectively), in the absence of any 2 MB acid added exogenously.

### Conclusion

Using more than 30 heterologous enzymes and proteins from different plant and microbial origins (e.g. G. vaccaria, Q. saponaria, A. thaliana, S. vaccaria, Thermothelomyces thermophilus, Aspergillus nidulans, and Aspergillus terreus), the inventors have been able to reconstruct in yeast the metabolic pathway leading to the synthesis of QS-21-Xyl and QS-21-Api (the two main isomeric constituents present in the QS-21 fraction traditionally purified from the bark of Q. saponaria Molina tree) achieving, for the first time, the successful production of QS-21-Xyl and QS-21-Api in yeast.

### Example 5—Methods

### 5.1 Expression in N. Benthamiana

N. Benthamiana transient expression experiments were carried out as described in WO 2020/260475.

### 5.2 Yeast Engineering

Genes were assembled into pESC plasmids which contain two multiple cloning sites driven by Gal1p and Gal10p individually which are galactose-inducible promoters or under the Tet promoter with the tet repressor gene. Nucleotide sequences were codon-optimized for S. cerevisiae using the IDT online tool. Integration was performed by an in-house-developed CRISPR/Cas9 toolkit10. Integration was confirmed by colony PCR and confirmed strains were glycerol stocked and stored at −80° C.

### 5.3 Production and Metabolite Extraction

Production of sugars and QA derivatives was done first by streaking the glycerol stock of the desired yeast strain onto a YPD (yeast extract peptone 2% dextrose) plate and grown for about 20 h at 30° C. to obtain single colonies. Colonies were picked from the plate and cultured for 48 h in 5 mL YPD shaking at 200 rpm at 30° C. The cultures were then spun down and resuspended in equal volume YPGal (yeast extract peptone 2% galactose) media and cultured further at 200 rpm and 30° C., inducing expression of Gall and Gall 0 promoters. Samples were collected at between 48 h and 36 hours post-induction for metabolite extraction. Yeast cell cultures (or cell pellet for the production of sugars) were extracted with 2:2:1 methanol/chloroform/water (2:2:1 v/v/v). Aqueous and organic layers were separated by centrifugation and the aqueous layer was collected. The collected layer was then evaporated in a speed vac at room temperature and resuspended in 0.3% formic acid at pH 9 (adjusted with ammonium acetate).

5.4 LC-MS Detection

LC-MS analysis was carried out using an Agilent HPLC 1260 infinity system attached to an iQ MSD. Detection: MS (ESI ionization, spray voltage Positive 4.5 kV, Negative −3.5 kV, mass range 400-1000, negative ion mode) LC Method: Solvent A: [H2O+0.3% formic acid at pH 9 (pH adjusted with ammonium hydroxide)] Solvent B: [acetonitrile (CH3CN)+0.1% formic acid]. Injection volume: 5 μL. Gradient: 2% to 15% [B] from 0 to 20 min, 15% to

50% [B] from 20 to 26 min, 50% to 90% [B] from 26 to 27 min, 90% [B] from 27 to 30 min, 90% to 2% [B] from 30 to 31 min, 2% [B] from 31 to 50 min. Method was performed using a flow rate of 0.1 mL min-1 with a Porous Graphitic Carbon column (Hypercarb, 5 μm, 1×150 mm Analytical Column) (or as described in WO 22/136563).

TABLE 3
Genotype of the engineered YL yeast strains

| | Parent | |
|---|---|---|
| Strain | strain | Genotype |
| CEN.PK2-1c | | MATa; his3-1; leu2-3_112; ura:3-52; trp1-2; MAL2-8c; |
| | | SUC2 |
| GTy23 | Cen.pk2-1c | erg9::KanMX_pCTR3-ERG9; leu2-3, |
| | | 112::His3MX6_pGal1-ERG19/pGal10-ERG8; ura3- |
| | | 52::URA3_pGal1-mvaS/pGal10- mvaE; |
| | | his3Δ1::hphMX4_pGal1-ERG12/pGal10-IDI1 |
| JWy601 | GTy23 | ura3-52 prototrophy removed for use of Cas9 system |
| MLY-01 | JWy601 | 1114a:pGal1-ERG20, 308a:pGal1-GvBAS |
| YL-1 | MLY-01 | 607c::pGal1-QsC28, pGal10-AtAtr1 |
| YL-2 | YL-1 | 416d::pGal1-QsC23 |
| YL-3 | YL-1 | 416d::pGal1-QsC23, pGal10-Qsb5 |
| YL-4 | YL-3 | 911b::pGal1-QsC28C16 |
| YL-5 | YL-4 | 911b::pGal1-QsC23C16 |
| YL-6 | YL-4 | 805a::pGal10-SvMSBP1 |
| YL-7 | YL-4 | 805a::pGal1-QsC28, pGal10-AtAtr1 |
| YL-8 | YL-6 | 1414a::pGal1-QsC28, pGal10AtAtr1 |
| YL-9 | YL-6 | rDNA::pGal1-QsC28, pGal10-AtAtr1 |
| YL-10 | YL-6 | 1414a::pGal1-QsC28, pGal10-AtAtr1; pGal1-QsC23, |
| | | pGal10-Qsb5; pGal1-QsC28C16, pGal10-SvMSBP1 |
| YL-11 | YL-10 | 106a::pGal1-QsCslG1, pGal10-AtUGD |
| YL-12 | YL-10 | 106a::pGal1-QsCslG2, pGal10-AtUGD |
| YL-13 | YL-10 | 106a::pGal1-QsCslG2, pGal10-AtUGD; pGal1-QsGalT |
| YL-14 | YL-10 | 106a::pGal1-QsCslG2, pGal10-AtUGD; pGal1-QsGalT; |
| | | pGal1-QsRhaT, pGal10-AtRHM2 |
| YL-15 | YL-10 | 106a::pGal1-QsCslG2, pGal10-AtUGD_{A101L}; pGal1- |
| | | QsGalT; pGal1-QsC3XylT, pGal10-AtUXS |
| YL-16 | YL-10 | 106a::pGal1-QsCslG2, pGal10-SynUGD; pGal1-QsGalT; |
| | | pGal1-C3XylT, pGal10-AtUXS |
| YL-17 | YL-10 | 106a::pGal1-QsCslG2, pGal10-HsUGD_{A104L}; pGal1- |
| | | QsGalT; pGal1-QsC3XylT, pGal10-AtUXS |
| YL-18 | YL-10 | 106a::pGal1-QsCslG2, pGal10-PatlUGD; pGal1-QsGalT; |
| | | pGal1-QsC3XylT, pGal10-AtUXS |
| YL-19 | YL-10 | 106a::pGal1-QsCslG2, pGal10-BcytUGD; pGal1-QsGalT; |
| | | pGal1-QsC3XylT, pGal10-AtUXS |
| YL-20 | YL-10 | 106a::pGal1-QsCslG2, pGal10-MyxfulvUGD; pGal1- |
| | | QsGalT; pGal1-QsC3XylT, pGal10-AtUXS |
| YL-21 | YL-10 | 106a::pGal1-QsCslG2, pGal10-PfuUGD; pGal1-QsGalT; |
| | | pGal1-QsC3XylT, pGal10-AtUXS |
| YL-22 | YL-15 | 106a::pGal1-QsCslG2, pGal10- AtUGD_{A101L}; pGal1- |
| | | QsGalT; pGal1- C3XylT, pGal10-AtUXS; 208a::pGal1- |
| | | AtUSP, pGal10-AtGlcAK |
| YL-23 | YL-15 | 106a::pGal1-QsCslG2, pGal10- AtUGD_{A101L}; pGal1- |
| | | QsGalT; pGal1- QsC3XylT, pGal10-AtUXS; 208a::pGal1- |
| | | AtUSP, pGal10-AtGlcAK; pGal1-TtMIOX |
| YL-24 | YL-10 | 106a::pGal1-QsCslG2, pGal10-AtUGD; pGal1-QsGalT; |
| | | pGal1- QsC3XylT, pTetO3-AtUXS |
| YL-25 | YL-14 | 1021b::pGal1-SvNMD, pGal10-SvUG46DH; pGal1- |
| | | QsFucT |
| YL-26 | YL-15 | 1021b::pGal1-SvNMD, pGal10-SvUG46DH; pGal1- |
| | | QsFucT |
| YL-27 | YL-14 | 1021b::pGal1-SvNMD, pGal10-SvUG46DH; pGal1- |
| | | QsFucT; pGal1-QsRhaT, pGal10-AtRHM2 |
| YL-28 | YL-15 | 1021b::pGal1-SvNMD, pGal10-SvUG46DH; pGal1- |
| | | QsFucT; pGal1-QsRhaT, pGal10-AtRHM2 |
| YL-29 | YL-27 | 208a::pGal1-QsC28XylT3, pGal10-AtUXS |
| YL-30 | YL-28 | 208a::pGal1-QsC28XylT3, pGal10-AtUXS |
| YL-31 | YL-29 | 1206a::pGal1-QsC28XylT4, pGal10-AtUXS |
| YL-32 | YL-29 | 1206a::pGal1-QsC28ApiT4, pGal10-AXS |
| YL-33 | YL-30 | 1206a::pGal1-QsC28XylT4, pGal10-AtUXS |
| YL-34 | YL-30 | 1206a::pGal1-QsC28ApiT4, pGal10-AXS |
| YL-35 | YL-30 | 1206a::pGal1-QsC28XylT4-6aa, pGal10-AtUXS |
| YL-36 | YL-30 | 1206a::pGal1-QsC28XylT4-9aa, pGal10-AtUXS |
| YL-37 | YL-30 | 1206a::pGal1-QsC28XylT4-12aa, pGal10-AtUXS |
| YL-38 | YL-30 | 1206a::pGal1-SUMO-QsC28XylT4, pGal10-AtUXS |
| YL-39 | YL-30 | 1206a::pGal1-TrxA-QsC28XylT4, pGal10-AtUXS |
| YL-40 | YL-30 | 1206a::pGal1-MBP-QsC28XylT4, pGal10-AtUXS |
| YL-41 | YL-30 | 1206a::pGal1-QsC28XylT3-3xGGGS- C28XylT4, pGal10- |
| | | AtUXS |
| YL-42 | YL-30 | YPRdelta::pGal1-QsChSD, pGal10-QsChSE; pGal1- |
| | | QsChSD, pGal10-QsChSE; pGal1-QsKR23, pGal10- |
| | | QsKR11; pGal1-QsCCL, pGal10-QsDMOT9 |
| YL-43 | YL-42 | 1206a::pGal1-QsDMOT4 |
| YL-44 | YL-42 | 1206a::pGal10-QsDMOT4, pGal1-QsC28XylT4 |
| YL-45 | YL-42 | 1206a::pGal10-AtUXS, pGal1-QsC28XylT4; pGal10- |
| | | AtUXE, pGal1-AtUAM1; pGal10-QsDMOT4, pGal1- |
| | | QsArafT |
| YL-46 | YL-42 | 1206a::pGal10-AtUXS, pGal1-QsC28ApiT4; pGal10- |
| | | AtUXE, pGal1- AtUAM1; pGal10-QsDMOT4, pGal1- |
| | | QsArafT |
| YL-47 | YL-42 | 1206a::pGal10-QsDMOT4, pGal1-QsArafT |
| YL-48 | YL-42 | 1206a::pGal10-QsDMOT4, pGal1-QsArafT2 |
| YL-49 | YL-42 | 1206a::pGal10-AtUXS, pGal1-QsC28XylT4; pGal10- |
| | | AtUXE, pGal1 - AtUAM1; pGal10-QsDMOT4, pGal1- |
| | | QsArafT2 |
| YL-50 | YL-45 | 720a::pADH-AnNgpA, pGal1-AstLovF-TE |
| YL-51 | YL-46 | 720a::pADH-AnNgpA, pGal1-AstLovF-TE |
| YL-QsCCL | Cen.pk2-1c | pESC::pGal10-QsCCL |
| YL-AnNpgA | Cen.pk2-1c | δ::pADH-AnNpgA |
| YL-PKS | YL-NpgA | pESC::pGal10-QsCCL, pGal1-AstLovF-TE |
| YL-42- | YL-42 | 720a::pADH-AnNgpA, pGal1-AstLovF-TE |
| AstLovF-TE | | |

Qs—Q. saponaria
Sv—S. vaccaria
Hs—Homo sapiens
Patl—Paramoeba atlantica
Pfu—Pyrococcus furiosus
An—Aspergillus nidulans
Gv—Gypsophila vaccaria
At—A. thaliana
Syn—Synechococcus
Bcyt—Bacillus cytotoxicus
Myxfulv—Corallococcus macrosporus
Tt—Thermothelomyces thermophilus
Ast—Aspergillus terreus

TABLE 4
Genotypes of the engineered SC yeast strains

| | Parent | |
|---|---|---|
| Strain | strain | Genotype |
| CEN.PK2-1c | | MATa; his3-1; leu2-3_112; ura:3-52; trp1-2; |
| | | MAL2-8c; SUC2 |
| SC-1 | Cen.pk2-1c | 1021b::pGal1-AtUGD |
| SC-2 | Cen.pk2-1c | 1021b::pGal1-AtUGD_{A101L} |
| SC-3 | Cen.pk2-1c | 1021b::pGal1-PatlUGD |
| SC-4 | Cen.pk2-1c | 1021b::pGal1-AtUGD, pGal10-Atos |
| SC-5 | Cen.pk2-1c | 1021b::pGal1-PatlUGD, pGal10-Atos |
| SC-6 | Cen.pk2-1c | 1021b::pGal1-AtUGD_{A101L}, pGal10-Atos |
| SC-7 | Cen.pk2-1c | 1021b::pGal1-AtUGD, pTetO3-Atos |
| SC-8 | Cen.pk2-1c | 1021b::pGal1- AtUGD_{A101}, pTetO3-Atos |
| SC-9 | SC-4 | 1414a::pGal1-AtUXE2, pGal10-AtUAM1 |
| SC-10 | SC-4 | 1414a::pGal1-HvUAM, pGal10-AtUXE2 |
| SC-11 | SC-4 | 1414a::pGal1-AtUAM1, pGal10-AtUGE3 |
| SC-12 | Cen.pk2-1c | 1414a::pGal1-AtUSP, pGal10-AtAraK |
| SC-13 | Cen.pk2-1c | 1414a::pGal1-LeiUSP, pGal10-LeiAraK |
| SC-14 | Cen.pk2-1c | 1414a::pGal1-AtUSP, pGal10-AtAraK, |
| | | pGal10-PrAraT |
| SC-15 | Cen.pk2-1c | 1414a::pGal1-AtUSP, pGal10-AtAraK, |
| | | pGal10-PrAraT, pGal1-AtUAM1 |
| SC-16 | Cen.pk2-1c | 1021b::pGal1-AtUGD, pGal10-QsAXS |
| SC-17 | Cen.pk2-1c | 416d::pGal1-AtRHM2 |
| SC-18 | SC-4 | 416d::pGal1-AtRHM2 |
| SC-19 | Cen.pk2-1c | 1206a::pGal1-SvUG46DH, pGal1-SvNMD |
| SC-20 | SC-4 | 1206a::pGal1-SvUG46DH, pGal1-SvNMD |
| SC-21 | SC-4 | 1206a::pGal1-SvUG46DH, pGal1-QsFucSyn |
| SC-22 | SC-17 | 1206a::pGal1-SvUG46DH, pGal1-SvNMD |
| SC-23 | SC-18 | 1206a::pGal1-SvUG46DH, pGal1-SvNMD |

Lei—Leptospira interrogans
Pr—Penicillium rubens Wisconsin
Hv—Hordeum vulgare

TABLE 5
Enzymes

| | | | Amino | |
|---|---|---|---|---|
| | | | acid | Nt |
| | | | SEQ | SEQ |
| Name | Enzyme type | Species origin | ID NO | ID NO |
| AaBAS | β-amyrin synthase | Artemisia annua | 1 | 2 |
| AtBAS | β-amyrin synthase | Arabidopsis thaliana | 4 | 5 |
| GgBAS | β-amyrin synthase | Glycyrrhiza glabra | 7 | 8 |
| GvBAS | β-amyrin synthase | Gypsophila vaccaria | 10 | 11 |
| SvBAS | β-amyrin synthase | Saponaria vaccaria | 13 | 14 |
| QsBAS | β-amyrin synthase | Quillaja saponaria | 15 | 16 |
| BfC16 | Cytochrome P450 C16 | Bupleurum falcatum | 17 | 18 |
| | oxidase | | | |
| QsC16 | Cytochrome P450 C16 | Quillaja saponaria | 20 | 21 |
| | oxidase | | | |
| QsC28C16 | Fusion protein (TM | Synthetic | 23 | 24 |
| | domain of | | | |
| | QsC28/QsC16) | | | |
| SvC16 | Cytochrome P450 C16 | Saponaria vaccaria | 26 | 27 |
| | oxidase | | | |
| QsC23 | Cytochrome P450 C23 | Quillaja saponaria | 29 | 30 |
| | oxidase | | | |
| SvC23-1 | Cytochrome P450 C23 | Saponaria vaccaria | 32 | 33 |
| | oxidase | | | |
| SvC23-2 | Cytochrome P450 C23 | Saponaria vaccaria | 35 | 36 |
| | oxidase | | | |
| MtC23 | Cytochrome P450 C23 | Medicago truncatula | 38 | 39 |
| | oxidase | | | |
| QsC28 | Cytochrome P450 C28 | Quillaja saponaria | 41 | 41 |
| | oxidase | | | |
| SvC28 | Cytochrome P450 C28 | Saponaria vaccaria | 44 | 45 |
| | oxidase | | | |
| MtC28 | Cytochrome P450 C28 | Medicago truncatula | 46 | 47 |
| | oxidase | | | |
| AtATR1 | Cytochrome P450 | Arabidopsis thaliana | 49 | 50 |
| | Reductase | | | |
| LjCPR | Cytochrome P450 | Lotus japonicus | 52 | 53 |
| | Reductase | | | |
| Qsb5 | Cytochrome | Quillaja saponaria | 55 | 56 |
| | protein/redox partner | | | |
| Atb5 | Cytochrome | Arabidopsis thaliana | 58 | 59 |
| | protein/redox partner | | | |
| Svb5 | Cytochrome | Saponaria vaccaria | 61 | 62 |
| | protein/redox partner | | | |
| AtMSBP1 | Scaffold protein | Arabidopsis thaliana | 63 | 64 |
| AtMSBP2 | Scaffold protein | Arabidopsis thaliana | 65 | 66 |
| SvMSBP1 | Scaffold protein | Saponaria vaccaria | 67 | 68 |
| SvMSBP2 | Scaffold protein | Saponaria vaccaria | 70 | 71 |
| QsMSBP1 | Scaffold protein | Quillaja saponaria | 73 | 74 |
| SvCslG | GlcA transferase | Saponaria vaccaria | 76 | 77 |
| QsCslG1 | GlcA transferase | Quillaja saponaria | 78 | 79 |
| QsCslG2 | GlcA transferase | Quillaja saponaria | 81 | 82 |
| AtUGD | UDP-glucose | Arabidopsis thaliana | 84 | 85 |
| | dehydrogenase | | | |
| SvUG46DH | UDP-glucose 4,6- | Saponaria vaccaria | 87 | 88 |
| | dehydratase | | | |
| SvNMD | 4-keto-reductase | Saponaria vaccaria | 90 | 91 |
| QsFucT | Fucosyltransferase | Quillaja saponaria | 93 | 94 |
| SvFucT | Fucosyltransferase | Saponaria vaccaria | 96 | 97 |
| SvGalT | Galactosyltransferase | Saponaria vaccaria | 98 | 99 |
| SvC3XylT | Xylosyltransferase | Saponaria vaccaria | 100 | 101 |
| AtRHM2 | Rhamnose synthase | Arabidopsis thaliana | 102 | 103 |
| AtUXS | Xylose synthase | Arabidopsis thaliana | 105 | 106 |
| AtUGD_{A101L} | UDP-glucose | Synthetic | 108 | 109 |
| | dehydrogenase | | | |
| PatlUGD | UDP-glucose | Paramoeba atlantica | 110 | 111 |
| | dehydrogenase | | | |
| QsAXS | UDP-apiose synthase | Quillaja saponaria | 113 | 114 |
| QsGalT | Galactosyltransferase | Quillaja saponaria | 116 | 117 |
| QsRhaT | Rhamnosyltransferase | Quillaja saponaria | 119 | 120 |
| QsC3XylT | UDP-xylose | Quillaja saponaria | 122 | 123 |
| | transferase | | | |
| QsC28XylT3 | UDP-xylose | Quillaja saponaria | 125 | 126 |
| | transferase | | | |
| QsC28XylT4 | UDP-xylose | Quillaja saponaria | 128 | 129 |
| | transferase | | | |
| QsC28XylT4- | UDP-xylose | Artificial | 131 | 132 |
| 3 aa | transferase truncated | | | |
| QsC28XylT4- | UDP-xylose | Artificial | 134 | 135 |
| 6 aa | transferase truncated | | | |
| QsC28XylT4- | UDP-xylose | Artificial | 137 | 138 |
| 9 aa | transferase truncated | | | |
| QsC28XylT4- | UDP-xylose | Artificial | 140 | 141 |
| 12 aa | transferase truncated | | | |
| SUMO- | UDP-xylose | Artificial | 143 | 144 |
| QsC28XylT4 | transferase solubility | | | |
| | tagged | | | |
| TrXA- | UDP-xylose | Artificial | 145 | 146 |
| QsC28XylT4 | transferase solubility | | | |
| | tagged | | | |
| MBP- | UDP-xylose | Artificial | 147 | 148 |
| QsC28XylT4 | transferase solubility | | | |
| | tagged | | | |
| QsC28XylT3- | UDP-xylose | Artificial | 149 | 150 |
| 3xGGGS- | transferase fusion | | | |
| QsC28XylT4 | variant | | | |
| QsC28ApiT4 | UDP-apiose | Quillaja saponaria | 151 | 152 |
| | transferase | | | |
| SynUGD | UDP-glucose | Synechococcus sp. | 154 | 155 |
| | dehydrogenase | | | |
| HsUGD_{A104L} | UDP-glucose | Artificial | 157 | 158 |
| | dehydrogenase | | | |
| BcytUGD | UDP-glucose | Bacillus cytotoxicus | 160 | 161 |
| | dehydrogenase | | | |
| MxfulvUGD | UDP-glucose | Corallococcusmacrosporu [symbol] | 163 | 164 |
| | dehydrogenase | | | |
| PfuUGD | UDP-glucose | Pyrococcus furiosus | 166 | 167 |
| | dehydrogenase | | | |
| AtGlcAK | Glucurokinase | Arabidopsis thaliana | 169 | 170 |
| TtMIOX | Myo-inositol | Thermothelomycesthermophilu [symbol] | 172 | 173 |
| | oxygenase | | | |
| QsFucSyn | Fucose reductase | Quillaja saponaria | 175 | 176 |
| QsCCL | CoA ligase | Quillaja saponaria | 178 | 179 |
| QsChSD | Chalcone-synthase- | Quillaja saponaria | 181 | 182 |
| | like type III PKS | | | |
| QsChSE | Chalcone-synthase- | Quillaja saponaria | 184 | 185 |
| | like type III PKS | | | |
| QsKR11 | Keto-reductase | Quillaja saponaria | 187 | 188 |
| QsKR23 | Keto-reductase | Quillaja saponaria | 190 | 191 |
| QsDMOT9 | Acyltransferase | Quillaja saponaria | 193 | 194 |
| QsDMOT4 | Acyltransferase | Quillaja saponaria | 196 | 197 |
| AtUXE | UDP-Xyl epimerase | Arabidopsis thaliana | 199 | 200 |
| AtUXE2 | UDP-Xyl epimerase | Arabidopsis thaliana | 202 | 203 |
| AtUGE3 | UDP-glucose 4- | Arabidopsis thaliana | 205 | 206 |
| | epimerase | | | |
| AtUAM1 | UDP-Ara mutase | Arabidopsis thaliana | 208 | 209 |
| HvUAM | UDP-Ara mutase | Hordeum vulgare | 211 | 212 |
| AtAraK | Arabinokinase | Arabidopsis thaliana | 214 | 215 |
| LeiAraK | Arabinokinase | Leptospira interrogans | 217 | 218 |
| PrAraT | Arabinose transporter | Penicillium rubens Wisconsin | 220 | 221 |
| AtUSP | UDP-sugar | Arabidopsis thaliana | 223 | 224 |
| | pyrophosphorylase | | | |
| LeiUSP | UDP-sugar | Leptospira interrogans | 226 | 227 |
| | pyrophosphorylase | | | |
| QsArafT | Arabinofuranose | Quillaja saponaria | 229 | 230 |
| | transferase | | | |
| QsArafT2 | Arabinofuranose | Quillaja saponaria | 232 | 233 |
| | transferase | | | |
| LovF-TE | Type I PKS | Artificial | 235 | 236 |
| | megasynthase | | | |
| AnNpgA | Phosphopantetheinyl | Aspergillus nidulans | 237 | 238 |
| | transferase | | | |
| HvUXE-1 | UDP-glucose 4- | Hordeum vulgare | 240 | 241 |
| | epimerase | | | |
| HvUXE-2 | UDP-glucose 4- | Hordeum vulgare | 242 | 243 |
| | epimerase | | | |

[symbol]

indicates data missing or illegible when filed

### REFERENCES

Kensil et al. (1991) “Separation and characterization of saponins with adjuvant activity from Quillaja saponaria Molina cortex”; Journal of immunology, Vol. 146: p 431-437
Ragupathi et al. (2011) “Natural and synthetic saponin adjuvant QS-21 for vaccines against cancer”; Expert Review of Vaccines, Vol. 10: p 463-470)
Garcon et al. “Recent clinical experience with vaccines using MPL and QS-21-containing adjuvant systems”; Expert Review of Vaccines, Vol. 10(4): p 71-486
Decker and Kleczkowski (2017) “Substrate Specificity and Inhibitor Sensitivity of Plant UDP-Sugar Producing Pyrophosphorylases”; Frontiers in Plant Science, Vol. 8(1610): p 1-16
Kirby et al. (2008)
Wong et al. (2018)
Gosh, 2017
WO19/106192
WO 19/122259
WO 20/260475
WO 22/136563
WO 20/263524

1. A method of producing in yeast quillaic acid (QA), or a QA derivative, or UDP-Fucose (UDP-Fuc), or (S)-2-methylbutyryl CoA (2 MB-CoA), or UDP-Arabinofuranose (UDP-Araf), the method selected from:
A) a method of producing quillaic acid (QA) in yeast, wherein the method comprises the step of overexpressing, in a yeast engineered to produce β-amyrin, heterologous genes encoding the following enzymes:
(i) a cytochrome P450 C16 oxidase, wherein the C16 oxidase oxidizes the C16 carbon of β-amyrin to a hydroxyl group,
(ii) a cytochrome P450 C23 oxidase, wherein the C23 oxidase oxidizes the C23 carbon of β-amyrin to an aldehyde group,
(iii) a cytochrome P450 C28 oxidase, wherein the C28 oxidase oxidizes the C28 carbon of β-amyrin to a carboxyl group, and
(iv) a cytochrome P450 reductase (CPR), acting as a redox partner,
wherein the C16 oxidase, the C23 oxidase, the C28 oxidase and the CPR are from a plant origin;
wherein, optionally,
(1)
(a) the C16 oxidase is selected from QsC16 according to SEQ ID NO: 20, QsC28C16 according to SEQ ID NO: 23, and SvC16 according to SEQ ID NO: 26, the C23 oxidase is selected from MtC23 oxidase according to SEQ ID NO: 38, QsC23 according to SEQ ID NO: 29, SvC23-1 according to SEQ ID NO: 32, and SvC23-2 according to SEQ ID NO: 35, and the C28 oxidase is selected from MtC28 according to SEQ ID NO: 46, QsC28 according to SEQ ID NO: 41 and SvC28 according to SEQ ID NO: 44; or
(b) the yeast further overexpresses a heterologous gene encoding (v) a cytochrome b5; or
(c) the yeast further overexpresses a heterologous gene encoding (vi) a scaffold protein, wherein the scaffold protein physically interacts with one or more of the C16 oxidase, the C23 oxidase, the C28 oxidase and the CPR, wherein the scaffold protein is a membrane steroid-binding protein (MSBP) selected from AtMSBP1 according to SEQ ID NO: 63, AtMSBP2 according to SEQ ID NO: 65, QsMSBP1 according to SEQ ID NO: 73, SvMSBP1 according to SEQ ID NO: 67 and SvMSBP2 according to SEQ ID NO: 70; and/or
(2) the yeast is engineered to produce β-amyrin and overexpresses a β-amyrin synthase (BAS) selected from AaBAS according to SEQ ID NO: 1, AtBAS according to SEQ ID NO: 4, GgBAS according to SEQ ID NO: 7, GvBAS according to SEQ ID NO: 10, QsBAS according to SEQ ID NO: 15, and SvBAS according to SEQ ID NO: 13, wherein, optionally, the C16 oxidase is QsC28C16, the C23 oxidase is QsC23, the C28 oxidase is QsC28, the CPR is AtATR1, the MSBP is SvMSBP1, the cytochrome b5 is Qsb5, and the BAS is GvBAS;
B) a method of producing a C3-glycosylated QA derivative in yeast, wherein the derivative is QA-C3-GlcA, and the method comprises the step of overexpressing, in a yeast engineered to produce QA and UDP-GlcA, a heterologous gene encoding the following enzyme: (i) a UDP-GlcA transferase (GlcAT) transferring UDP-GlcA and attaching a GlcA residue at the C3 position of QA to form QA-C3-GlcA wherein, optionally,
(1) (a) the GlcAT is selected from QsCslG1 according to SEQ ID NO: 78, QsCslG2 according to SEQ ID NO: 81, and SvCslG according to SEQ ID NO: 76; or
(b) the derivative is QA-C3-GlcA-Gal, and the overexpressing further comprises overexpressing a heterologous gene encoding the following enzyme: (ii) a UDP-Galactose transferase (GalT) transferring UDP-Gal and attaching a Gal residue to QA-C3-GlcA to form QA-C3-GlcA-Gal, wherein, optionally, the GalT is QsGalT according to SEQ ID NO: 116 or GalT is SvGalT according to SEQ ID NO: 98; or
(c) the derivative is QA-C3-GlcA-Gal-Rha, the yeast is further engineered to produce UDP-Rha, and the overexpressing further comprises overexpressing a heterologous gene encoding the following enzyme: (iii) a UDP-Rhamnose transferase (RhaT) transferring UDP-Rha and attaching a Rha residue to QA-C3-GlcA-Gal to form QA-C3-GlcA-Gal-Rha.
(2) the derivative is QA-C3-GlcA-Gal-Xyl, the yeast is further engineered to produce UDP-Xyl, and the overexpressing further comprises overexpressing heterologous genes encoding the following enzymes:
a UDP-Xylose transferase (XylT) transferring UDP-Xylose and attaching a Xyl residue to QA-C3-GlcA-Gal to form QA-C3-GlcA-Gal-Xyl,
wherein, optionally, the XylT is selected from QsC3XylT according to SEQ ID NO: 122 and SvC3XylT according to SEQ ID NO: 100; and/or
C) a method of producing UDP-Fucose (UDP-Fuc) in yeast, wherein the method comprises the step of overexpressing heterologous genes encoding the following enzymes:
(i) a UDP-glucose-4,6-dehydratase (UG46DH) converting UDP-Glc into UDP-4-keto-6-deoxy-glucose and
(ii) a 4-keto-reductase converting UDP-4-keto-6-deoxy-glucose into UDP-D-Fuc;
wherein, optionally,
the UG46DH is SvUG46DH according to SEQ ID NO: 87 and the 4-keto-reductase is selected from svNMD according to SEQ ID NO: 90 and QsFucSyn according to SEQ ID NO: 175;
D) a method of producing a C-28-glycosylated QA derivative in yeast, wherein the derivative is QA-C3-GlcA-Gal-Rha-C28-Fuc, or QA-C3-GlcA-Gal-Xyl-C28-Fuc, the method comprises the step of overexpressing, in a yeast engineered to produce QA-C3-GlcA-Gal-Rha, or QA-C3-GlcA-Gal-Xyl, and UDP-Fucose, a heterologous gene encoding the following enzyme: (i) a UDP-Fucose transferase (FucT) transferring UDP-Fuc and attaching a Fuc residue at the C28 position of QA to form QA-C3-GlcA-Gal-Rha-C28-Fuc, or QA-C3-GlcA-Gal-Xyl-C28-Fuc, wherein, optionally,
(1) the FucT is selected from QsFucT according to SEQ ID NO: 93 and SvFucT according to SEQ ID NO: 96; or
(2) the derivative is QA-C3-GlcA-Gal-Rha-C28-Fuc-Rha, or QA-C3-GlcA-Gal-Xyl-C28-Fuc-Rha, the overexpressing further comprises overexpressing a heterologous gene encoding the following enzyme: (ii) a UDP-Rhamnose transferase (RhaT) transferring UDP-Rha and attaching a Rha residue to QA-C3-GlcA-Gal-Rha-C28-Fuc, or QA-C3-GlcA-Gal-Xyl-C28-Fuc, to form QA-C3-GlcA-Gal-Rha-C28-Fuc-Rha or QA-C3-GlcA-Gal-Xyl-C28-Fuc-Rha, wherein, optionally,
(2a) the RhaT is QsRhaT according to SEQ ID NO: 119; or
(2b) the derivative is QA-C3-GlcA-Gal-Rha-C28-Fuc-Rha-Xyl, or QA-C3-GlcA-Gal-Xyl-C28-Fuc-Rha-Xyl, the overexpressing further comprises overexpressing heterologous genes encoding the following enzyme: (iii) a UDP-Xylose transferase (XylT) transferring UDP-Xyl and attaching a Xyl residue to QA-C3-GlcA-Gal-Rha-C28-Fuc-Rha or QA-C3-GlcA-Gal-Xyl-C28-Fuc-Rha to form GlcA-Gal-Rha-C28-Fuc-Rha-Xyl and QA-C3-GlcA-Gal-Xyl-C28-Fuc-Rha-Xyl, respectively, wherein, optionally,
(2b1) the XylT is QsC28XylT3 according to SEQ ID NO: 125; or
(2b2) the derivative is QA-C3-GlcA-Gal-Rha-C28-Fuc-Rha-Xyl-Xyl, or QA-C3-GlcA-Gal-Xyl-C28-Fuc-Rha-Xyl-Xyl, the overexpressing further comprises overexpressing heterologous genes encoding the following enzymes: (iv) a UDP-Xylose transferase (XylT) transferring UDP-Xyl and attaching a Xyl residue to QA-C3-GlcA-Gal-Rha-C28-Fuc-Rha-Xyl or QA-C3-GlcA-Gal-Xyl-C28-Fuc-Rha-Xyl to form QA-C3-GlcA-Gal-Rha-C28-Fuc-Rha-Xyl-Xyl and QA-C3-GlcA-Gal-Xyl-C28-Fuc-Rha-Xyl-Xyl, respectively, wherein, optionally,
(2b2.1) the XylT is QsC28XylT4 according to SEQ ID NO: 128; or
(2b2.2) the derivative is QA-C3-GlcA-Gal-Rha-C28-Fuc-Rha-Xyl-Api or QA-C3-GlcA-Gal-Xyl-C28-Fuc-Rha-Xyl-Api, the overexpressing further comprises overexpressing heterologous genes encoding the following enzymes: (iv) a UDP-Apiose synthase (AXS) converting UDP-GlcA into UDP-Api and (v) a UDP-Apiose transferase (ApiT) transferring UDP-Apiose and attaching an Apiose residue to QA-C3-GlcA-Gal-Rha-C28-Fuc-Rha-Xyl or QA-C3-GlcA-Gal-Xyl-C28-Fuc-Rha-Xyl to form QA-C3-GlcA-Gal-Rha-C28-Fuc-Rha-Xyl-Api and QA-C3-GlcA-Gal-Xyl-C28-Fuc-Rha-Xyl-Api, respectively, wherein, optionally, the AXS is QsAXS according to SEQ ID NO: 113 and the ApiT is QsC28ApiT4 according to SEQ ID NO: 151;
E) a method of producing (S)-2-methylbutyryl CoA (2 MB-CoA) in yeast, wherein the method comprises the step of overexpressing a heterologous gene encoding a carboxyl coenzyme A (CoA) ligase (CCL) converting 2 MB acid into 2 MB-CoA, and 2 MB acid is supplemented exogenously; wherein, optionally,
(1) the CCL is QsCCL from Q. saponaria according to SEQ ID NO: 178, or
(2) the overexpressing further comprises overexpressing heterologous genes encoding the following enzymes:
(i) a phosphopantetheinyl (Ppant) transferase,
(ii) a megasynthase LovF-TE including an ACP domain, condensing two units of malonyl-CoA to 2 MB-ACP, cleaving 2 MB acid from the ACP domain which is converted into 2 MB-CoA by the CCL,
and no 2 MB acid is supplemented exogenously, wherein, optionally,
the Ppant is AnNpgA according to SEQ ID NO: 237 and the megasynthase LovF-TE is AstLovF-TE according to SEQ ID NO: 235;
F) a method of producing UDP-Arabinofuranose (UDP-Araf) in yeast, wherein the method comprises the step of overexpressing, in a yeast engineered to produce UDP-Xyl, heterologous genes encoding the following enzymes:
(i) a UDP-Xyl epimerase (UXE) converting UDP-Xyl into UDP-Arabinopyranose (UDP-Arap), and
(ii) a UDP-Arabinose mutases (UAM) converting UDP-Arap into UDP-Arabinofuranose (UDP-Ara),
wherein, optionally, the UXE is selected from AtUXE according to SEQ ID NO: 199, AtUXE2 according to SEQ ID NO: 202, HvUXE-1 according to SEQ ID NO: 240, HvUXE-2 according to SEQ ID NO: 242 and AtUGE3 according to SEQ ID NO: 205 and the UAM is selected from AtUAM1 according to SEQ ID NO: 208 and HvUAM according to SEQ ID NO: 211;
G) a method of producing UDP-Araf in yeast, wherein the method comprises the step of overexpressing heterologous genes encoding the following enzymes:
(i) an arabinokinase (AraK) and
(ii) a UDP-sugar pyrophosphorylase (USP),
and arabinose is supplemented exogenously,
wherein, optionally,
the AraK is selected from AtAraK according to SEQ ID NO: 214 and LeiAraK according to SEQ ID NO: 217 and the USP is selected from AtUSP according to SEQ ID NO: 223 and LeiUSP according to SEQ ID NO: 226, and wherein, optionally, the overexpressing further comprises overexpressing a heterologous gene encoding an arabinose transporter (AraT), wherein AraT is optionally encoded by the nucleotide sequence SEQ ID NO: 221.
H) a method of producing an acylated and glycosylated QA derivative in yeast, wherein the derivative is QA-C3-GGR-C28-FRX-C9, QA-C3-GGX-C28-FRX-C9, QA-C3-GGR-C28-FRXX-C9, QA-C3-GGX-C28-FRXX-C9, QA-C3-GGR-C28-FRXA-C9 or QA-C3-GGX-C28-FRXA-C9, and the method comprises the step of overexpressing, in a yeast engineered to produce QA-C3-GGR-C28-FRX, QA-C3-GGX-C28-FRX, QA-C3-GGR-C28-FRXX, QA-C3-GGX-C28-FRXX, QA-C3-GGR-C28-FRXA, or QA-C3-GGX-C28-FRXA, heterologous genes encoding the following enzymes:
(i) a carboxyl coenzyme A ligase (CCL) converting 2 MB acid into 2 MB-CoA,
(ii) a chalcone-synthase-like type III PKS (Polyketide synthase) condensing malonyl-CoA with 2 MB-CoA to form C9-Keto-CoA,
(iii) a keto-reductase (KR) converting C9-Keto-CoA into C9-CoA, and
(iv) an acyltransferase transferring and attaching a first C9-CoA unit to QA-C3-GGR-C28-FRX, QA-C3-GGX-C28-FRX, QA-C3-GGR-C28-FRXX, QA-C3-GGX-C28-FRXX, QA-C3-GGR-C28-FRXA, or QA-C3-GGX-C28-FRXA to form QA-C3-GGR-C28-FRX-C9, QA-C3-GGX-C28-FRX-C9, QA-C3-GGR-C28-FRXX-C9, QA-C3-GGX-C28-FRXX-C9, QA-C3-GGR-C28-FRXA-C9 or QA-C3-GGX-C28-FRXA-C9. wherein 2 MB acid is supplemented exogenously;
I) a method of producing QA-C3-GGX-C28-FRX-C18-Xyl, QA-C3-GGR-C28-FRX-C18-Xyl, QA-C3-GGX-C28-FRXX-C18-Xyl, QA-C3-GGR-C28-FRXX-C18-Xyl, QA-C3-GGX-C28-FRX-C18-Xyl, QA-C3-GGX-C28-FRXA-C18-Xyl or QA-C3-GGR-C28-FRX-C18-Xyl in a yeast, wherein the method comprises the step of overexpressing, in a yeast engineered to produce QA-C3-GGX-C28-FRX-C18, QA-C3-GGR-C28-FRX-C18, QA-C3-GGX-C28-FRXX-C18, QA-C3-GRX-C28-FRXX-C18, QA-C3-GGX-C28-FRX-C18, QA-C3-GGX-C28-FRXA-C18 or QA-C3-GGR-C28-FRX-C18, a heterologous gene encoding an arabinotransferase (ArafT) transferring UDP-Xyl and attaching a Xyl residue to QA-C3-GGX-C28-FRX-C18, QA-C3-GGR-C28-FRX-C18, QA-C3-GGX-C28-FRXX-C18, QA-C3-GRX-C28-FRXX-C18, QA-C3-GGX-C28-FRX-C18, QA-C3-GGX-C28-FRXA-C18 and QA-C3-GGR-C28-FRX-C18 to form QA-C3-GGX-C28-FRX-C18-Xyl, QA-C3-GGR-C28-FRX-C18-Xyl, QA-C3-GGX-C28-FRXX-C18-Xyl, QA-C3-GRX-C28-FRXX-C18-Xyl, QA-C3-GGX-C28-FRX-C18-Xyl, QA-C3-GGX-C28-FRXA-C18-Xyl or QA-C3-GGR-C28-FRX-C18-Xyl; wherein, optionally,
the ArafT is QsArafT according to SEQ ID NO: 229; and
J) a method of producing QA-C3-GGX-C28-FRXX-C18-Araf (QS-21-Xyl) or QA-C3-GGX-C28-FRXA-C18-Araf (QS-21-Api) in yeast, wherein
(1) the method of producing QA-C3-GGX-C28-FRXX-C18-Araf (QS-21-Xyl) comprises the step of overexpressing heterologous genes encoding GvBAS according to SEQ ID NO: 10, QsC28C16 according to SEQ ID NO: 23, QsC23 according to SEQ ID NO: 29, QsC28 according to SEQ ID NO: 41, AtATR1 according to SEQ ID NO: 49, Qsb5 according to SEQ ID NO: 55, SvMSBP1 according to SEQ ID NO: 67, AtUGD_{A101L} according to SEQ ID NO: 108, QsCslG2 according to SEQ ID NO: 78, QsGalT according to SEQ ID NO: 116, AtUXS according to SEQ ID NO: 105, QsC3XylT according to SEQ ID NO: 122, SvNMD according to SEQ ID NO: 90, SvUG46DH according to SEQ ID NO: 87, QsFuct according to SEQ ID NO: 93, AtRHM2 according to SEQ ID NO: 102, QsRhaT according to SEQ ID NO: 119, QsC28XylT3 according to SEQ ID NO: 125, QsC28XylT4 according to SEQ ID NO: 128, QsChSD according to SEQ ID NO: 181, QsChSE according to SEQ ID NO: 184, QsKR11 according to SEQ ID NO: 187, QsKR23 according to SEQ ID NO: 190, QsDMOT9 according to SEQ ID NO: 193, QsDMOT4 according to SEQ ID NO: 196, AtUXE according to SEQ ID NO: 199, AtUAM1 according to SEQ ID NO: 208, QsArafT2 according to SEQ ID NO: 232, AnNpgA according to SEQ ID NO: 237, QsCCL according to SEQ ID NO: 178 and AstLovF-TE according to SEQ ID NO: 235; and
(2) the method of producing QA-C3-GGX-C28-FRXA-C18-Araf (QS-21-Api) comprises the step of overexpressing heterologous genes encoding GvBAS according to SEQ ID NO: 10, QsC28C16 according to SEQ ID NO: 23, QsC23 according to SEQ ID NO: 29, QsC28 according to SEQ ID NO: 41, AtATR1 according to SEQ ID NO: 49, Qsb5 according to SEQ ID NO: 55, SvMSBP1 according to SEQ ID NO: 67, AtUGD_{A101L} according to SEQ ID NO: 108, QsCslG2 according to SEQ ID NO: 81, QsGalT according to SEQ ID NO: 116, AtUXS according to SEQ ID NO: 105, QsC3XylT according to SEQ ID NO: 122, SvNMD according to SEQ ID NO: 90, SvUG46DH according to SEQ ID NO: 87, QsFucT according to SEQ ID NO: 93, AtRHM2 according to SEQ ID NO: 102, QsRhaT according to SEQ ID NO: 119, QsC28XylT3 according to SEQ ID NO: 125, QsC28ApiT4 according to SEQ ID NO: 151, QsChSD according to SEQ ID NO: 181, QsChSE according to SEQ ID NO: 184, QsKR11 according to SEQ ID NO: 187, QsKR23 according to SEQ ID NO: 190, QsDMOT9 according to SEQ ID NO: 193, QsDMOT4 according to SEQ ID NO: 196, AtUXE according to SEQ ID NO: 199, AtUAM1 according to SEQ ID NO: 208, QsArafT2 according to SEQ ID NO: 232, AnNpgA according to SEQ ID NO: 237, QsCCL according to SEQ ID NO: 178 and AstLovF-TE according to SEQ ID NO: 235.

2. The method of claim 1, comprising a method of producing quillaic acid (QA) in yeast, wherein the method comprises the step of overexpressing, in a yeast engineered to produce β-amyrin, heterologous genes encoding the following enzymes:
(i) a cytochrome P450 C16 oxidase, wherein the C16 oxidase oxidizes the C16 carbon of β-amyrin to a hydroxyl group,
(ii) a cytochrome P450 C23 oxidase, wherein the C23 oxidase oxidizes the C23 carbon of β-amyrin to an aldehyde group,
(iii) a cytochrome P450 C28 oxidase, wherein the C28 oxidase oxidizes the C28 carbon of β-amyrin to a carboxyl group, and
(iv) a cytochrome P450 reductase (CPR), acting as a redox partner wherein the C16 oxidase, the C23 oxidase, the C28 oxidase and the CPR are from a plant origin;
wherein, optionally,
(1)
(a) the C16 oxidase is selected from QsC16 according to SEQ ID NO: 20, QsC28C16 according to SEQ ID NO: 23, and SvC16 according to SEQ ID NO: 26, the C23 oxidase is selected from MtC23 oxidase according to SEQ ID NO: 38, QsC23 according to SEQ ID NO: 29, SvC23-1 according to SEQ ID NO: 32, and SvC23-2 according to SEQ ID NO: 35, and the C28 oxidase is selected from MtC28 according to SEQ ID NO: 46, QsC28 according to SEQ ID NO: 41 and SvC28 according to SEQ ID NO: 44; or
(b) the yeast further overexpresses a heterologous gene encoding (v) a cytochrome b5; or
(c) the yeast further overexpresses a heterologous gene encoding (vi) a scaffold protein, wherein the scaffold protein physically interacts with one or more of the C16 oxidase, the C23 oxidase, the C28 oxidase and the CPR, wherein the scaffold protein is a membrane steroid-binding protein (MSBP) selected from AtMSBP1 according to SEQ ID NO: 63, AtMSBP2 according to SEQ ID NO: 65, QsMSBP1 according to SEQ ID NO: 73, SvMSBP1 according to SEQ ID NO: 67 and SvMSBP2 according to SEQ ID NO: 70; and/or
(2) the yeast is engineered to produce β-amyrin and overexpresses a β-amyrin synthase (BAS) selected from AaBAS according to SEQ ID NO: 1, AtBAS according to SEQ ID NO: 4, GgBAS according to SEQ ID NO: 7, GvBAS according to SEQ ID NO: 10, QsBAS according to SEQ ID NO: 15, and SvBAS according to SEQ ID NO: 13, wherein, optionally, the C16 oxidase is QsC28C16, the C23 oxidase is QsC23, the C28 oxidase is QsC28, the CPR is AtATR1, the MSBP is SvMSBP1, the cytochrome b5 is Qsb5, and the BAS is GvBAS.

3. The method of claim 1, comprising a method of producing a C3-glycosylated QA derivative in yeast, wherein the derivative is QA-C3-GlcA, and the method comprises the step of overexpressing, in a yeast engineered to produce QA and UDP-GlcA, a heterologous gene encoding the following enzyme: (i) a UDP-GlcA transferase (GlcAT) transferring UDP-GlcA and attaching a GlcA residue at the C3 position of QA to form QA-C3-GlcA, wherein, optionally,
(1) (a) the GlcAT is selected from QsCslG1 according to SEQ ID NO: 78, QsCslG2 according to SEQ ID NO: 81, and SvCslG according to SEQ ID NO: 76; or
(b) the derivative is QA-C3-GlcA-Gal, and the overexpressing further comprises overexpressing a heterologous gene encoding the following enzyme: (ii) a UDP-Galactose transferase (GalT) transferring UDP-Gal and attaching a Gal residue to QA-C3-GlcA to form QA-C3-GlcA-Gal, wherein, optionally, the GalT is QsGalT according to SEQ ID NO: 116 or GalT is SvGalT according to SEQ ID NO: 98; or
(c) the derivative is QA-C3-GlcA-Gal-Rha, the yeast is further engineered to produce UDP-Rha, and the overexpressing further comprises overexpressing a heterologous gene encoding the following enzyme: (iii) a UDP-Rhamnose transferase (RhaT) transferring UDP-Rha and attaching a Rha residue to QA-C3-GlcA-Gal to form QA-C3-GlcA-Gal-Rha; and/or
(2) the derivative is QA-C3-GlcA-Gal-Xyl, the yeast is further engineered to produce UDP-Xyl, and the overexpressing further comprises overexpressing heterologous genes encoding the following enzymes:
a UDP-Xylose transferase (XylT) transferring UDP-Xylose and attaching a Xyl residue to QA-C3-GlcA-Gal to form QA-C3-GlcA-Gal-Xyl,
wherein, optionally, the XylT is selected from QsC3XylT according to SEQ ID NO: 122 and SvC3XylT according to SEQ ID NO: 100.

4. The method of claim 1, comprising a method of producing UDP-Fucose (UDP-Fuc) in yeast, wherein the method comprises the step of overexpressing heterologous genes encoding the following enzymes:
(i) a UDP-glucose-4,6-dehydratase (UG46DH) converting UDP-Glc into UDP-4-keto-6-deoxy-glucose and
(ii) a 4-keto-reductase converting UDP-4-keto-6-deoxy-glucose into UDP-D-Fuc;
wherein, optionally,
the UG46DH is SvUG46DH according to SEQ ID NO: 87 and the 4-keto-reductase is selected from svNMD according to SEQ ID NO: 90 and QsFucSyn according to SEQ ID NO: 175.

5. The method of claim 1, comprising a method of producing a C-28-glycosylated QA derivative in yeast, wherein the derivative is QA-C3-GlcA-Gal-Rha-C28-Fuc, or QA-C3-GlcA-Gal-Xyl-C28-Fuc, the method comprises the step of overexpressing, in a yeast engineered to produce QA-C3-GlcA-Gal-Rha, or QA-C3-GlcA-Gal-Xyl, and UDP-Fucose, a heterologous gene encoding the following enzyme: (i) a UDP-Fucose transferase (FucT) transferring UDP-Fuc and attaching a Fuc residue at the C28 position of QA to form QA-C3-GlcA-Gal-Rha-C28-Fuc, or QA-C3-GlcA-Gal-Xyl-C28-Fuc,
wherein, optionally,
(1) the FucT is selected from QsFucT according to SEQ ID NO: 93 and SvFucT according to SEQ ID NO: 96; or
(2) the derivative is QA-C3-GlcA-Gal-Rha-C28-Fuc-Rha, or QA-C3-GlcA-Gal-Xyl-C28-Fuc-Rha, the overexpressing further comprises overexpressing a heterologous gene encoding the following enzyme: (ii) a UDP-Rhamnose transferase (RhaT) transferring UDP-Rha and attaching a Rha residue to QA-C3-GlcA-Gal-Rha-C28-Fuc, or QA-C3-GlcA-Gal-Xyl-C28-Fuc, to form QA-C3-GlcA-Gal-Rha-C28-Fuc-Rha or QA-C3-GlcA-Gal-Xyl-C28-Fuc-Rha, wherein, optionally,
(2a) the RhaT is QsRhaT according to SEQ ID NO: 119; or
(2b) the derivative is QA-C3-GlcA-Gal-Rha-C28-Fuc-Rha-Xyl, or QA-C3-GlcA-Gal-Xyl-C28-Fuc-Rha-Xyl, the overexpressing further comprises overexpressing heterologous genes encoding the following enzyme: (iii) a UDP-Xylose transferase (XylT) transferring UDP-Xyl and attaching a Xyl residue to QA-C3-GlcA-Gal-Rha-C28-Fuc-Rha or QA-C3-GlcA-Gal-Xyl-C28-Fuc-Rha to form GlcA-Gal-Rha-C28-Fuc-Rha-Xyl and QA-C3-GlcA-Gal-Xyl-C28-Fuc-Rha-Xyl, respectively, wherein, optionally,
(2b1) the XylT is QsC28XylT3 according to SEQ ID NO: 125; or
(2b2) the derivative is QA-C3-GlcA-Gal-Rha-C28-Fuc-Rha-Xyl-Xyl, or QA-C3-GlcA-Gal-Xyl-C28-Fuc-Rha-Xyl-Xyl, the overexpressing further comprises overexpressing heterologous genes encoding the following enzymes: (iv) a UDP-Xylose transferase (XylT) transferring UDP-Xyl and attaching a Xyl residue to QA-C3-GlcA-Gal-Rha-C28-Fuc-Rha-Xyl or QA-C3-GlcA-Gal-Xyl-C28-Fuc-Rha-Xyl to form QA-C3-GlcA-Gal-Rha-C28-Fuc-Rha-Xyl-Xyl and QA-C3-GlcA-Gal-Xyl-C28-Fuc-Rha-Xyl-Xyl, respectively, wherein, optionally,
(2b2.1) the XylT is QsC28XylT4 according to SEQ ID NO: 128; or
(2b2.2) the derivative is QA-C3-GlcA-Gal-Rha-C28-Fuc-Rha-Xyl-Api or QA-C3-GlcA-Gal-Xyl-C28-Fuc-Rha-Xyl-Api, the overexpressing further comprises overexpressing heterologous genes encoding the following enzymes: (iv) a UDP-Apiose synthase (AXS) converting UDP-GlcA into UDP-Api and (v) a UDP-Apiose transferase (ApiT) transferring UDP-Apiose and attaching an Apiose residue to QA-C3-GlcA-Gal-Rha-C28-Fuc-Rha-Xyl or QA-C3-GlcA-Gal-Xyl-C28-Fuc-Rha-Xyl to form QA-C3-GlcA-Gal-Rha-C28-Fuc-Rha-Xyl-Api and QA-C3-GlcA-Gal-Xyl-C28-Fuc-Rha-Xyl-Api, respectively, wherein, optionally, the AXS is QsAXS according to SEQ ID NO: 113 and the ApiT is QsC28ApiT4 according to SEQ ID NO: 151.

6. The method of claim 1, comprising a method of producing (S)-2-methylbutyryl CoA (2 MB-CoA) in yeast, wherein the method comprises the step of overexpressing a heterologous gene encoding a carboxyl coenzyme A (CoA) ligase (CCL) converting 2 MB acid into 2 MB-CoA, and 2 MB acid is supplemented exogenously;
wherein, optionally,
(1) the CCL is QsCCL from Q. saponaria according to SEQ ID NO: 178, or
(2) the overexpressing further comprises overexpressing heterologous genes encoding the following enzymes:
(i) a phosphopantetheinyl (Ppant) transferase,
(ii) a megasynthase LovF-TE including an ACP domain, condensing two units of malonyl-CoA to 2 MB-ACP, cleaving 2 MB acid from the ACP domain which is converted into 2 MB-CoA by the CCL,
and no 2 MB acid is supplemented exogenously, wherein, optionally,
the Ppant is AnNpgA according to SEQ ID NO: 237 and the megasynthase LovF-TE is AstLovF-TE according to SEQ ID NO: 235.

7. The method of claim 1, comprising a method of producing UDP-Arabinofuranose (UDP-Araf) in yeast, wherein the method comprises the step of overexpressing, in a yeast engineered to produce UDP-Xyl, heterologous genes encoding the following enzymes:
(i) a UDP-Xyl epimerase (UXE) converting UDP-Xyl into UDP-Arabinopyranose (UDP-Arap), and
(ii) a UDP-Arabinose mutases (UAM) converting UDP-Arap into UDP-Arabinofuranose (UDP-Ara),
wherein, optionally, the UXE is selected from AtUXE according to SEQ ID NO: 199, AtUXE2 according to SEQ ID NO: 202, HvUXE-1 according to SEQ ID NO: 240, HvUXE-2 according to SEQ ID NO: 242 and AtUGE3 according to SEQ ID NO: 205 and the UAM is selected from AtUAM1 according to SEQ ID NO: 208 and HvUAM according to SEQ ID NO: 211.

8. The method of claim 1, comprising a method of producing UDP-Araf in yeast, wherein the method comprises the step of overexpressing heterologous genes encoding the following enzymes:
(i) an arabinokinase (AraK) and
(ii) a UDP-sugar pyrophosphorylase (USP),
and arabinose is supplemented exogenously,
wherein, optionally,
the AraK is selected from AtAraK according to SEQ ID NO: 214 and LeiAraK according to SEQ ID NO: 217 and the USP is selected from AtUSP according to SEQ ID NO: 223 and LeiUSP according to SEQ ID NO: 226, and wherein, optionally, the overexpressing further comprises overexpressing a heterologous gene encoding an arabinose transporter (AraT), wherein AraT is optionally encoded by the nucleotide sequence SEQ ID NO: 221.

9. The method of claim 1, comprising a method of producing an acylated and glycosylated QA derivative in yeast, wherein the derivative is QA-C3-GGR-C28-FRX-C9, QA-C3-GGX-C28-FRX-C9, QA-C3-GGR-C28-FRXX-C9, QA-C3-GGX-C28-FRXX-C9, QA-C3-GGR-C28-FRXA-C9 or QA-C3-GGX-C28-FRXA-C9, and the method comprises the step of overexpressing, in a yeast engineered to produce QA-C3-GGR-C28-FRX, QA-C3-GGX-C28-FRX, QA-C3-GGR-C28-FRXX, QA-C3-GGX-C28-FRXX, QA-C3-GGR-C28-FRXA, or QA-C3-GGX-C28-FRXA,
heterologous genes encoding the following enzymes:
(i) a carboxyl coenzyme A ligase (CCL) converting 2 MB acid into 2 MB-CoA,
(ii) a chalcone-synthase-like type III PKS (Polyketide synthase) condensing malonyl-CoA with 2 MB-CoA to form C9-Keto-CoA,
(iii) a keto-reductase (KR) converting C9-Keto-CoA into C9-CoA, and
(iv) an acyltransferase transferring and attaching a first C9-CoA unit to QA-C3-GGR-C28-FRX, QA-C3-GGX-C28-FRX, QA-C3-GGR-C28-FRXX, QA-C3-GGX-C28-FRXX, QA-C3-GGR-C28-FRXA, or QA-C3-GGX-C28-FRXA to form QA-C3-GGR-C28-FRX-C9, QA-C3-GGX-C28-FRX-C9, QA-C3-GGR-C28-FRXX-C9, QA-C3-GGX-C28-FRXX-C9, QA-C3-GGR-C28-FRXA-C9 or QA-C3-GGX-C28-FRXA-C9.
wherein 2 MB acid is supplemented exogenously.

10. The method of claim 9, wherein
(1) the CCL is QsCCL according to SEQ ID NO: 178, the chalcone-synthase-like type III PKS is QsChSD according to SEQ ID NO: 181, QsChSE according to SEQ ID NO: 184, or both QsChSD according to SEQ ID NO:181 and QsChSE according to SEQ ID NO: 184, the keto-reductase is QsKR11 according to SEQ ID NO: 187, QsKR23 according to SEQ ID NO: 190, or both QsKR11 according to SEQ ID NO: 187 and QsKR23 according to SEQ ID NO: 190, and the acyltransferase is QsDMOT9 according to SEQ ID NO: 193; or
(2) the derivative is QA-C3-GGR-C28-FRX-C18, QA-C3-GGX-C28-FRX-C18, QA-C3-GGR-C28-FRXX-C18, QA-C3-GGX-C28-FRXX-C18, QA-C3-GGR-C28-FRXA-C18 or QA-C3-GGX-C28-FRXA-C18, and the overexpressing further comprises overexpressing a heterologous gene encoding the following enzyme: (v) an acyltransferase QsDMOT4 according to SEQ ID NO: 196 attaching a second C9-CoA unit to C3-GGR-C28-FRX-C9, QA-C3-GGX-C28-FRX-C9, QA-C3-GGR-C28-FRXX-C9, QA-C3-GGX-C28-FRXX-C9, QA-C3-GGR-C28-FRXA-C9, or QA-C3-GGX-C28-FRXA-C9 to form C3-GGR-C28-FRX-C18, QA-C3-GGX-C28-FRX-C18, QA-C3-GGR-C28-FRXX-C18, QA-C3-GGX-C28-FRXX-C18, QA-C3-GGR-C28-FRXA-C18 or QA-C3-GGX-C28-FRXA-C18, wherein, optionally,
(2a) QsDMOT4 is encoded by the nucleotide sequence SEQ ID NO: 197; or
(2b) the derivative is QA-C3-GGR-C28-FRX-C18-Araf, QA-C3-GGX-C28-FRX-C18-Araf, QA-C3-GGR-C28-FRXX-C18-Araf, QA-C3-GGX-C28-FRXX-C18-Araf, QA-C3-GGR-C28-FRXA-C18-Araf, or QA-C3-GGX-C28-FRXA-C18-Araf, the yeast is further engineered to produce UDP-Araf, and the overexpressing further comprises overexpressing a heterologous gene encoding the following enzyme: (vi) an arabinotransferase (ArafT) transferring UDP-Araf and attaching an Araf residue to QA-C3-GGR-C28-FRX-C18, QA-C3-GGX-C28-FRX-C18, QA-C3-GGR-C28-FRXX-C18, QA-C3-GGX-C28-FRXX-C18, QA-C3-GGR-C28-FRXA-C18, or QA-C3-GGX-C28-FRXA-C18- to form QA-C3-GGR-C28-FRX-C18-Araf, QA-C3-GGX-C28-FRX-C18-Araf, QA-C3-GGR-C28-FRXX-C18-Araf, QA-C3-GGX-C28-FRXX-C18-Araf, QA-C3-GGR-C28-FRXA-C18-Araf or QA-C3-GGX-C28-FRXA-C18-Araf, wherein, optionally, the ArafT is selected from QsArafT according to SEQ ID NO: 229 and QsArafT2 according to SEQ ID NO: 232, the ArafT optionally being QsArafT2 according to SEQ ID NO: 232.

11. The method of claim 1, comprising a method of producing QA-C3-GGX-C28-FRX-C18-Xyl, QA-C3-GGR-C28-FRX-C18-Xyl, QA-C3-GGX-C28-FRXX-C18-Xyl, QA-C3-GGR-C28-FRXX-C18-Xyl, QA-C3-GGX-C28-FRX-C18-Xyl, QA-C3-GGX-C28-FRXA-C18-Xyl or QA-C3-GGR-C28-FRX-C18-Xyl in a yeast, wherein the method comprises the step of overexpressing, in a yeast engineered to produce QA-C3-GGX-C28-FRX-C18, QA-C3-GGR-C28-FRX-C18, QA-C3-GGX-C28-FRXX-C18, QA-C3-GRX-C28-FRXX-C18, QA-C3-GGX-C28-FRX-C18, QA-C3-GGX-C28-FRXA-C18 or QA-C3-GGR-C28-FRX-C18, a heterologous gene encoding an arabinotransferase (ArafT) transferring UDP-Xyl and attaching a Xyl residue to QA-C3-GGX-C28-FRX-C18, QA-C3-GGR-C28-FRX-C18, QA-C3-GGX-C28-FRXX-C18, QA-C3-GRX-C28-FRXX-C18, QA-C3-GGX-C28-FRX-C18, QA-C3-GGX-C28-FRXA-C18 and QA-C3-GGR-C28-FRX-C18 to form QA-C3-GGX-C28-FRX-C18-Xyl, QA-C3-GGR-C28-FRX-C18-Xyl, QA-C3-GGX-C28-FRXX-C18-Xyl, QA-C3-GRX-C28-FRXX-C18-Xyl, QA-C3-GGX-C28-FRX-C18-Xyl, QA-C3-GGX-C28-FRXA-C18-Xyl or QA-C3-GGR-C28-FRX-C18-Xyl;
wherein, optionally,
the ArafT is QsArafT according to SEQ ID NO: 229.

12. The method of claim 10, wherein the overexpressing further comprises the overexpressing of heterologous genes encoding the following enzymes:
(i) a phosphopantetheinyl (Ppant) transferase,
(ii) a megasynthase LovF-TE including an ACP domain, condensing two units of malonyl-CoA to 2 MB-ACP, cleaving 2 MB acid from the ACP domain which is converted into 2 MB-CoA by the CoA ligase (CCL), and no 2 MB acid is supplemented exogenously,
wherein, optionally,
the Ppant is AnNpgA according to SEQ ID NO: 237 and the megasynthase LovF-TE is AstLovF-TE according to SEQ ID NO: 235.

13. The method of claim 11, wherein the overexpressing further comprises the overexpressing of heterologous genes encoding the following enzymes:
(i) a phosphopantetheinyl (Ppant) transferase,
(ii) a megasynthase LovF-TE including an ACP domain, condensing two units of malonyl-CoA to 2 MB-ACP, cleaving 2 MB acid from the ACP domain which is converted into 2 MB-CoA by the CoA ligase (CCL), and no 2 MB acid is supplemented exogenously, wherein, optionally,
the Ppant is AnNpgA according to SEQ ID NO: 237 and the megasynthase LovF-TE is AstLovF-TE according to SEQ ID NO: 235.

14. The method of claim 1, comprising a method of producing QA-C3-GGX-C28-FRXX-C18-Araf (QS-21-Xyl) or QA-C3-GGX-C28-FRXA-C18-Araf (QS-21-Api) in yeast, wherein
(1) the method of producing QA-C3-GGX-C28-FRXX-C18-Araf (QS-21-Xyl) comprises the step of overexpressing heterologous genes encoding GvBAS according to SEQ ID NO: 10, QsC28C16 according to SEQ ID NO: 23, QsC23 according to SEQ ID NO: 29, QsC28 according to SEQ ID NO: 41, AtATR1 according to SEQ ID NO: 49, Qsb5 according to SEQ ID NO: 55, SvMSBP1 according to SEQ ID NO: 67, AtUGD_{A101L} according to SEQ ID NO: 108, QsCslG2 according to SEQ ID NO: 78, QsGalT according to SEQ ID NO: 116, AtUXS according to SEQ ID NO: 105, QsC3XylT according to SEQ ID NO: 122, SvNMD according to SEQ ID NO: 90, SvUG46DH according to SEQ ID NO: 87, QsFuct according to SEQ ID NO: 93, AtRHM2 according to SEQ ID NO: 102, QsRhaT according to SEQ ID NO: 119, QsC28XylT3 according to SEQ ID NO: 125, QsC28XylT4 according to SEQ ID NO: 128, QsChSD according to SEQ ID NO: 181, QsChSE according to SEQ ID NO: 184, QsKR11 according to SEQ ID NO: 187, QsKR23 according to SEQ ID NO: 190, QsDMOT9 according to SEQ ID NO: 193, QsDMOT4 according to SEQ ID NO: 196, AtUXE according to SEQ ID NO: 199, AtUAM1 according to SEQ ID NO: 208, QsArafT2 according to SEQ ID NO: 232, AnNpgA according to SEQ ID NO: 237, QsCCL according to SEQ ID NO: 178 and AstLovF-TE according to SEQ ID NO: 235; and
(2) the method of producing QA-C3-GGX-C28-FRXA-C18-Araf (QS-21-Api) comprises the step of overexpressing heterologous genes encoding GvBAS according to SEQ ID NO: 10, QsC28C16 according to SEQ ID NO: 23, QsC23 according to SEQ ID NO: 29, QsC28 according to SEQ ID NO: 41, AtATR1 according to SEQ ID NO: 49, Qsb5 according to SEQ ID NO: 55, SvMSBP1 according to SEQ ID NO: 67, AtUGD_{A101L} according to SEQ ID NO: 108, QsCslG2 according to SEQ ID NO: 81, QsGalT according to SEQ ID NO: 116, AtUXS according to SEQ ID NO: 105, QsC3XylT according to SEQ ID NO: 122, SvNMD according to SEQ ID NO: 90, SvUG46DH according to SEQ ID NO: 87, QsFucT according to SEQ ID NO: 93, AtRHM2 according to SEQ ID NO: 102, QsRhaT according to SEQ ID NO: 119, QsC28XylT3 according to SEQ ID NO: 125, QsC28ApiT4 according to SEQ ID NO: 151, QsChSD according to SEQ ID NO: 181, QsChSE according to SEQ ID NO: 184, QsKR11 according to SEQ ID NO: 187, QsKR23 according to SEQ ID NO: 190, QsDMOT9 according to SEQ ID NO: 193, QsDMOT4 according to SEQ ID NO: 196, AtUXE according to SEQ ID NO: 199, AtUAM1 according to SEQ ID NO: 208, QsArafT2 according to SEQ ID NO: 232, AnNpgA according to SEQ ID NO: 237, QsCCL according to SEQ ID NO: 178 and AstLovF-TE according to SEQ ID NO: 235.

15. Glycosylated QA derivative, wherein the glycosylated derivative is selected from (1) C3-glycosylated QA derivatives obtained according to the method of claim 3.

16. Glycosylated QA derivative, wherein the glycosylated derivative is selected from (2) C28—glycosylated QA derivatives obtained according to the method of claim 5.

17. Glycosylated QA derivative, wherein the glycosylated derivative is selected from (3) acylated and glycosylated QA derivatives obtained according to the method of claim 9.

18. The use of C3-glycosylated QA derivatives, C28-glycosylated QA derivatives, and acylated and glycosylated QA derivatives of claim 15 as an adjuvant.

19. An isolated polypeptide selected from a β-amyrin synthase (SvBAS) according to SEQ ID NO: 13, a β-amyrin synthase (QsBAS) according to SEQ ID NO: 15, a CYP C16 oxidase (QsC28C16) according to SEQ ID NO: 23, a CYP C16 oxidase (SvC16) according to SEQ ID NO: 26, CYP C23 oxidase (SvC23-1) according to SEQ ID NO: 32, a CYP C23 oxidase (SvC23-2) according to SEQ ID NO: 35, a CYP C28 oxidase (SvC28) according to SEQ ID NO: 44, ACytochrome b5 protein (Qsb5) according to SEQ ID NO: 55, a Cytochrome b5 protein (Svb5) according to SEQ ID NO: 61, a UDP-GlcA transferase (SvCslG) according to SEQ ID NO: 76, AMSBP protein (SvMSBP1) according to SEQ ID NO: 67, AMSBP protein (SvMSBP2) according to SEQ ID NO: 70, a MSBP protein (QsMSBP1) according to SEQ ID NO: 73, a UDP-glucose-4,6-dehydratase (SvUG46DH) according to SEQ ID NO: 87, a UDP-4-keto-6-deoxy-glucose reductase (SvNMD) according to SEQ ID NO: 90, a UDP-Galactose transferase (SvGalT) according to SEQ ID NO: 98, a UDP-Fucose transferase (SvFucT) according to SEQ ID NO: 96, a UDP-Xylose transferase (SvC3XylT) according to SEQ ID NO: 100, AUDP-Arabinofuranose transferase (QsArafT2) according to SEQ ID NO: 229, a UDP-glucose dehydrogenase (AtUGD_{A101L}) according to SEQ ID NO: 108, a UDP-Xylose transferase (QsC28XylT4-3aa) according to SEQ ID NO: 131, a AUDP-Xylose transferase (QsC28XylT4-6aa) according to SEQ ID NO: 134, a UDP-Xylose transferase (QsC28XylT4-9aa) according to SEQ ID NO: 137, a UDP-Xylose transferase (QsC28XylT4-12aa) according to SEQ ID NO: 140, AUDP-Xylose transferase (SUMO-QsC28XylT4) according to SEQ ID NO: 143, a UDP-Xylose transferase (TrXA-QsC28XylT4) according to SEQ ID NO: 145, a UDP-Xylose transferase (MBP-QsC28XylT4) according to SEQ ID NO: 147, a AUDP-Xylose transferase (QsC28XylT3-3×GGGS-QsC28XylT4) according to SEQ ID NO: 149 and a type I polyketide synthase (AstLovF-TE) according to SEQ ID NO: 235.


SEQUENCE LISTING (243 sequences)

SEQ ID NO: 1 - protein, 761 aa, Artemisia annua
MWRLKIAEGRNDPYLYSTNNFVGRQIWEFDPNYGTPEERAEVEQARVDFWNHRHEVKPSSDVLWRMQFLREKGFEQTIPQVKIEDGEEISYEKATTTLRRSVNFFAALQADDGHWPAENAGPLYFMQPLVICLYITGHLNTVFPAEYRKEILRYIYCHQNEDGGWGFHIEGHSTMFCTTLSYICMRLLGEGRDGGLDGACTKARKWILDHGSVTTIPSWGKTWLSILGVCEWAGTNPMPPEFWILPSFLPMYPAKMWCYCRLVYMPMSYLYGKRFVGPITPLILQLRDELYAQPYDEIKWRSIRHLCAKEDLYYPHPLLQDLMWDSLYVFTEPVLNHWPFNKLREKALQTTMKHIHYEDENSRYITIGSVEKALCMLACWVEDPNGVCFKKHIARIPDYLWVAEDGMKMQSFGSQEWDAGFAIQALMATDLTDEIGSTLMKGHEFIKASQVKDNPSGDFKSMHRHISKGSWTFSDQDHGWQVSDCTAEALKCCLLFATMPPEIVGEKMKPEQLNDAVNVILSLQSKNGGLAAWEPAGSSEWLEILNPTEFFADIVIEHEYVECTSSAIQALVMFKKKYPGHRKKEIENFLLGSSGYLEKIQMEDGSWYGNWGVCFTYGTWFALGGLSAVGKTYDNCPAIRKAVKFLLETQLEDGGWGESYKSCPEKKYIPLEGGRSNLVHTAWAMMGLIHSRQAERDATPLHRAAKLLINSQLETGDFPQQEIAGVFMKNCMLHYALYRNIYPMWALADYRKQVLPQLKGT

SEQ ID NO: 2 - DNA, 2286 bp, Artemisia annua
  source 1..2286: mol_type = genomic DNA
atgtggagattgaaaatagcagaagggcgcaatgatccgtacctgtacagcacaaacaactttgttggaaggcaaatatgggagtttgacccgaattatggaactccagaagagcgtgctgaggtggaacaagctcgtgttgatttctggaatcacagacacgaagttaaacccagtagtgatgttctttggcgcatgcagtttttaagagagaagggatttgaacagacgataccccaagtgaaaatagaggacggtgaagaaatatcgtatgaaaaagctacaaccacattaaggagatcggttaacttctttgcagctttgcaggccgatgatggtcactggccagctgaaaatgctggtcccttgtatttcatgcagcccttggtcatatgcttgtatattactgggcaccttaacactgttttcccagcagaatatcgaaaagaaattctacggtacatatactgtcatcagaatgaagacggtgggtggggatttcacattgagggtcatagtacaatgttctgcaccaccctcagttacatttgtatgcgtcttttgggggaaggtcgtgatggtggtctggatggtgcatgcaccaaagctcgtaaatggattctagaccatggcagtgtcacgaccattccatcttggggtaaaacatggctttcgatactcggtgtatgtgagtgggcaggaaccaacccaatgccccccgagttctggatcctcccttctttcttacctatgtatccagcaaaaatgtggtgttactgtcgacttgtatatatgccaatgtcatacctatacggaaaaagattcgtgggtcctattaccccattaattctacaacttagagatgaactctatgcacaaccgtatgatgaaattaaatggaggagcataaggcatttgtgtgcaaaggaagacttatactatcctcatcctttactacaagatttgatgtgggatagtctttatgtttttactgagcctgtactaaatcactggccatttaataaattgcgcgagaaagcgctacaaacaacaatgaagcatatccattatgaagatgaaaacagcagatatatcacaattggttctgtagagaaggcactgtgtatgctcgcatgctgggttgaggatccaaatggagtttgttttaaaaagcatattgctcgtattccagattatctctgggttgctgaagatggaatgaaaatgcagagcttcggcagtcaggagtgggatgctggttttgctattcaagctctaatggcaactgatcttactgatgaaattgggtctactttgatgaaaggacatgaatttattaaagcttcacaggttaaagacaatccttcgggtgactttaaaagcatgcatcgacatatttctaaagggtcatggacattttcagatcaagatcatggatggcaagtttcagactgtactgccgaagcattaaagtgttgcctcctatttgcaacgatgccaccagaaatagttggggagaaaatgaaacctgaacaactgaacgatgctgtcaacgtaatactttccttacagagcaaaaatggcgggctagccgcatgggaaccagcaggatcgtcagaatggctggagattctcaatcctacagagttctttgctgatatcgtcatagagcatgagtacgttgagtgcacatcatcagcaattcaggctctggttatgttcaaaaagaaatacccggggcataggaagaaagagatagaaaattttcttttgggttcaagtggatacctggagaaaatacagatggaagatggttcatggtatggaaactggggtgtgtgttttacatacggtacatggtttgctcttggaggactatcagcggttgggaaaacatatgacaattgtccagcaatccgtaaagctgtcaaatttctcctggaaacccagctggaagatggtggttggggagaaagctacaaatcctgtccagaaaagaaatatatacccctggaaggaggtcggtccaacttggtacacactgcatgggctatgatgggactaattcattctcgacaggccgagagagatgcaacacccctacacagagcagccaaactattaatcaactcccagttggaaactggtgattttccacaacaggaaatagctggagtctttatgaagaattgcatgctacactatgccttatacagaaatatatacccgatgtgggctctagctgattatcgcaagcaggtgctgccacagctcaaaggcacctag

SEQ ID NO: 3 - DNA, 2286 bp, synthetic construct
  source 1..2286: mol_type = other DNA
atgtggaggttgaagatagcagaaggaaggaatgatccatacctttactctacgaacaacttcgtgggaaggcagatatgggagtttgatccaaactatggaacgccggaagaaagggctgaagtggagcaggcgagggtagatttctggaatcatagacacgaagtcaagccgtctagtgacgtgttatggaggatgcaattcctgcgtgagaagggattcgaacaaacgataccccaagtaaagatagaagacggggaggaaattagttatgaaaaggctacaaccaccctgaggagatcagtaaatttttttgccgctttacaagctgatgatggacactggccagctgagaacgccggtccgctgtatttcatgcagccattagtgatctgcctttatatcacgggtcatttaaatacggtctttccagcagagtacagaaaagaaatacttagatatatttactgccatcaaaacgaggacggtggttggggatttcacatcgaggggcactccacaatgttctgtacgacgctttcctacatttgcatgcgtctgcttggagaaggtcgtgacggggggctggatggtgcctgcacaaaagctagaaagtggattcttgatcatggttctgtcacgactataccgtcatggggaaagacgtggctaagcattcttggggtatgcgagtgggctgggacaaatcccatgccaccggagttttggatcttaccgtcctttcttccaatgtaccccgcaaaaatgtggtgctattgtagattagtttatatgccgatgtcatatctttacggtaaacgtttcgtcggacccattacgcccctaattttacagcttagggacgagctttacgcgcaaccctacgatgaaatcaaatggcgttctatcagacatttgtgcgcgaaggaagatctgtactacccgcaccctcttcttcaagatctaatgtgggacagcctttatgtgtttactgaacccgtcttgaatcactggccattcaataagctgagggaaaaggctcttcagactactatgaaacatatacattatgaggatgaaaattcaagatacatcacgataggatcagtagaaaaagccttgtgcatgcttgcatgctgggtagaggacccgaacggcgtctgcttcaagaaacacatcgccagaatccccgattatctatgggtggcagaagatggcatgaaaatgcaaagtttcggaagccaggaatgggatgcgggtttcgcgatacaggcattaatggctacggatcttaccgatgaaatcgggtccacactgatgaaaggtcacgagtttattaaagcttctcaggtgaaggataatccgtccggcgacttcaagtctatgcatcgtcatattagcaaagggagctggacatttagtgatcaagatcacggatggcaggtatctgactgtactgcagaggcattaaaatgctgtttgctgtttgcgacgatgccacctgaaattgttggtgaaaaaatgaaacctgaacaactaaacgacgccgtaaatgtcattctttctctgcagagtaaaaatggtggccttgcagcatgggaacccgccggaagtagtgagtggctggagattctgaatcctacagaatttttcgccgatattgtcatagaacacgagtacgtcgaatgcacaagcagcgcgattcaagctttagtgatgtttaaaaagaaataccccggccacaggaagaaagagatcgagaattttttgctagggtcaagtggctacttagagaaaatccaaatggaagatggtagctggtatggcaactggggagtgtgttttacctatggaacatggtttgcgctgggtggcttatccgcggtaggcaaaacctacgacaattgccccgccatacgtaaggcggtcaaattcttgctggaaacccaacttgaggatggggggtggggagagtcctacaaaagctgcccagaaaagaagtatatccctttggaagggggaagatcaaacctagtccacacagcatgggccatgatgggcctgatccactcaaggcaagccgagagagatgctacaccactacacagggctgcgaaactgctgatcaacagccagctagagactggggactttccacagcaagagatagcgggtgtatttatgaagaattgcatgttgcattatgcgttgtataggaacatctacccgatgtgggccttggctgattacaggaaacaggtgttgccccaattaaaaggaacgtag

SEQ ID NO: 4 - protein, 759 aa, Arabidopsis thaliana
MWRLKIGEGNGDDPYLFTTNNFAGRQTWEFDPDGGSPEERHSVVEARRIFYDNRFHVKASSDLLWRMQFLREKKFEQRIAPVKVEDSEKVTFETATSALRRGIHFFSALQASDGHWPAENAGPLFFLPPLVFCLYITGHLDEVFTSEHRKEILRYIYCHQKEDGGWGLHIEGHSTMFCTTLNYICMRILGESPDGGHDNACGRAREWILSHGGVTYIPSWGKTWLSILGVFDWSGSNPMPPEFWILPSFFPVHPAKMWSYCRMVYLPMSYLYGKRFVGPITSLILQLRKELYLQPYEEINWMKVRHLCAKEDTYYPRPLVQELVWDSLYIFAELFLARWPFNKLLREKALQLAMKHIHYEDENSRYITIGCVEKVLCMLACWVEDPNGDYFKKHLSRISDYLWMAEDGMKMQSFGSQLWDTGFAMQALLASNLSSEISDVLRRGHEFIKNSQVGENPSGDYKSMYRHISKGAWTFSDRDHGWQVSDCTAHGLKCCLLFSMLAPDIVGPKQDPERLHDSVNILLSLQSKNGGMTAWEPAGAPKWSELLNPTEMFSDIVIEHEYSECTSSAIQALSLFKQLYPDHRTTEITAFIKKAAEYLENMQTRDGSWYGNWGICFTYGTWFALAGLAAAGKTFNDCEAIRKGVQFLLAAQKDNGGWGESYLSCSKKIYIAQVGEISNVVQTAWALMGLIHSGQAERDPIPLHRAAKLIINSQLESGDFPQQQATGVFLKNCTLHYAAYRNIHPLWALAEYRARVSLP

SEQ ID NO: 5 - DNA, 2280 bp, Arabidopsis thaliana
  source 1..2280: mol_type = genomic DNA
atgtggaggttgaagataggagaagggaacggagatgatccctacttattcactactaacaacttcgccggacgacaaacatgggagtttgatcctgacggaggctcgccggaggaaagacactccgtcgttgaggctcgccggattttctacgacaatcgttttcatgttaaagcaagcagcgatctcttgtggcgcatgcagtttctgagggagaaaaagtttgagcaacggatagctccggtgaaagttgaagactccgagaaggtaacctttgaaacggcgacgagtgcattacggagaggtattcatttcttctcggcgttgcaggccagcgacggtcactggccggcggaaaatgccggccctttgtttttccttccgccactggtgttttgtctttacataacgggacatcttgatgaagtgttcacttcagaacatcgaaaagagattcttcgatacatctactgtcaccagaaggaagatggtgggtggggattacacattgaaggtcatagcacgatgttctgtaccacgttgaattacatatgtatgcgtatacttggagaaagtcctgatggaggtcatgataatgcgtgtggacgagcccgggaatggatccttagtcatggtggtgtcacctacataccttcttggggtaaaacttggctttcgatacttggtgtatttgactggtctggaagcaacccaatgcctcctgagttttggatcctaccttctttctttcccgtgcatccagcaaaaatgtggagctactgccggatggtttacttgccaatgtcgtatctctacgggaagcggtttgttggcccaataacgtctctgattttgcaactacgcaaagaactgtacttacagccttatgaagaaatcaattggatgaaagtccgccatctttgtgcaaaggaagatacatattatccccgtccactggttcaagagttggtatgggacagtctttacatctttgcggagcttttccttgcacgttggccatttaacaagcttcttagggaaaaggctcttcaattggcaatgaaacacatacactatgaagatgaaaatagtcgttatatcaccattgggtgtgttgaaaaggtactatgcatgctagcttgttgggttgaagacccgaatggggattatttcaagaagcacctctctagaatctccgattacttgtggatggctgaagatgggatgaaaatgcagagctttggaagtcaactatgggatacagggtttgcgatgcaagctttacttgcaagcaatctctccagtgaaatctctgatgtactcaggagaggacatgagttcataaagaattcacaggttggagagaacccttcaggtgattacaaaagcatgtaccgtcatatttcaaaaggagcatggactttttctgaccgagatcacggatggcaagtttcagactgcacagctcatggcttaaagtgctgcctgctgttctcgatgttggcgccggatattgttggcccaaaacaagaccccgagagactacatgattctgttaatatcttgctctctttacagagcaaaaacggaggtatgactgcctgggagcctgctggtgctcccaagtggtcggaattgctcaatcccacagaaatgttttccgacattgtgattgagcatgagtacagtgaatgtacatcgtctgcaatccaagcattgagtcttttcaagcaactctaccctgatcacaggacaacagagatcaccgctttcatcaagaaagctgcggaatacctagaaaacatgcaaacacgtgatggttcatggtatgggaactggggcatttgcttcacgtacggtacatggtttgctcttgcaggcttagcagctgcgggtaagacttttaacgactgtgaggcaatacgcaaaggcgttcagtttcttcttgcagctcagaaagacaacggaggctggggagaaagctacctctcttgctccaaaaagatatacatagcacaagtaggggagatatcaaacgtggtgcaaactgcttgggctttaatgggtctcattcactctggacaggcagagagagatccgattcctcttcaccgtgctgcgaaacttatcatcaattcacaactggagagtggagattttcctcaacagcaagcaaccggagtgtttttgaagaattgcacattacactacgctgcatatagaaacattcatccgttgtgggcactcgcagaataccgcgcgcgagtttcgttgccatga

SEQ ID NO: 6 - DNA, 2280 bp, synthetic construct
  source 1..2280: mol_type = other DNA
atgtggcgtttgaaaattggcgagggaaacggcgatgatccctatttattcacaacaaacaatttcgctggtaggcagacatgggagtttgacccagacgggggtagtccagaagagcgtcactctgttgtagaggcgcgtaggatattctatgataatagatttcacgtaaaggcctccagcgatttattgtggaggatgcaatttcttagagagaaaaaatttgaacagaggatagccccggtgaaggtggaagacagcgagaaggtaacttttgagaccgctactagcgctctaagaagaggaattcacttcttttccgcgttgcaagcgtctgatggtcattggcctgcagagaacgccgggccgctgttcttcctgccgcctctggttttctgtctatatataaccggtcatctggatgaagtattcacctctgaacatagaaaagagatactacgttacatatactgtcatcagaaagaggacggtggttgggggcttcacatcgaaggacatagtaccatgttttgtacaacactaaattacatttgtatgagaattctgggggagtccccagatggaggacatgacaacgcttgtggcagggctagggaatggatcttaagccatggaggggtaacatatatacctagctggggaaagacctggttgagcattcttggtgtttttgactggtcaggtagcaacccgatgccgccagaattctggatcctaccctccttttttccggttcacccagccaagatgtggagctactgtcgtatggtctacctaccgatgtcctatttatacggcaaaagatttgtaggcccaatcacaagtctaatcttacagctgaggaaggaattgtatctgcagccctacgaagagattaattggatgaaagtgagacatctatgcgcgaaagaagatacctattatcctagaccgttggttcaggaattggtttgggatagcctttatatattcgcggagctgtttctagcgcgttggccatttaataagttgttgcgtgaaaaggccttacaactagccatgaagcatatccattacgaggatgagaattcaaggtatataacgattgggtgcgtcgaaaaggtattgtgcatgctggcttgctgggtggaagatcctaatggagattacttcaagaaacacctgtccagaattagtgactatctttggatggccgaagatgggatgaagatgcagtctttcggatctcagttgtgggatacaggtttcgcaatgcaggcacttttggctagcaacttatctagtgaaatcagcgatgttttaagacgtggacatgagttcataaaaaacagtcaagtgggcgaaaatccaagcggggactacaaaagtatgtacagacacattagcaaaggagcgtggactttctctgatagagatcatggatggcaagtttcagactgcacggcccatgggcttaaatgctgtcttctattcagtatgttagcccccgatatagtgggaccgaagcaagatcccgagaggctacatgattccgttaacatacttttatcattacagagtaagaacggtggaatgaccgcatgggagccagctggcgcccctaagtggtcagaattattaaaccctactgagatgttcagcgatatagttattgagcatgaatacagcgaatgcacctcttctgccatccaggcgctgtccttattcaagcagttgtaccctgatcaccgtacaacagagataactgccttcatcaaaaaggcagcggagtacctagagaatatgcagaccagagacggttcttggtatggtaattgggggatctgctttacgtatggcacctggttcgctttggctggcttggcggccgctggcaagacattcaatgattgcgaggccattcgtaagggcgtacaatttctactggccgcccagaaggacaatggaggttggggcgagtcatatttgtcttgttcaaagaaaatctacatagctcaagtcggagagatatcaaatgtagtacaaactgcatgggctttgatgggtctaattcattcagggcaagctgagcgtgatcctatccctctacatagagccgccaaactaataatcaactcccagttagaaagcggggattttcctcaacagcaagccacaggggtatttttaaaaaactgcactctgcattacgcggcttataggaacattcaccccttgtgggccctagctgaatacagggcacgtgtatccttgccgtga

SEQ ID NO: 7 - protein, 762 aa, Glycyrrhiza glabra
MWRLKIAEGGKDPYIYSTNNFVGRQTWEYDPGGGSAEERAQVDAARLHFYNNRFQVKPCGDLLWRFQILRENNFKQTIASVKIGDGEEITYEKATTAVRRAAHHLSALQTSDGHWPAQIAGPLFFLPPLVFCMYITGHLDSVFPEEYRKEILRYIYYHQNEDGGWGLHIEGHSTMFCTALNYICMRILGEGPDGGQDNACARARKWIHDHGGVTHIPSWGKTWLSILGVFDWCGSNPMPPEFWILPSFLPMHPAKMWCYCRLVYMPMSYLYGKRFVGPITPLILQLREELFTEPYEKVNWKKARHQCAKEDLYYPHPLLQDLIWDSLYLFTEPLLTRWPFNKLVREKALQVTMKHIHYEDETSRYITIGCVEKVLCMLACWVEDPNGDAFKKHLARVPDYLWVSEDGMTMQSFGSQEWDAGFAVQALLATNLVEEIAPTLAKGHDFIKKSQVRDNPSGDFKSMYRHISKGSWTFSDQDHGWQVSDCTAEGLKCCLLLSMLPPEIVGEKMEPERLYDSVNVLLSLQSKKGGLSAWEPAGAQEWLELLNPTEFFADIVVEHEYVECTGSAIQALVLFKKLYPGHRKKEIENFIANAVRFLEDTQTADGSWYGNWGVCFTYGSWFALGGLAAAGKTFANCAAIRKAVKFLLTTQREDGGWGESYLSSPKKIYVPLEGSRSNVVHTAWALMGLIHAGQAERDPAPLHRAAKLIINSQLEEGDWPQQEITGVFMKNCMLHYPMYRDIYPMWALAEYRRRVPLPSTPA

SEQ ID NO: 8 - DNA, 2289 bp, Glycyrrhiza glabra
  source 1..2289: mol_type = genomic DNA
atgtggaggctgaagatagcggaaggagggaaggacccatacatatacagcacaaacaacttcgtggggaggcagacatgggagtatgatcccggtggtgggagtgccgaggagagagctcaggttgatgccgctcgtctccatttctataacaaccgcttccaggtcaagccctgtggcgacctcctttggcgttttcagattctgagagaaaataacttcaaacaaacaatagctagcgtgaagataggagacggagaggaaataacatacgaaaaggcgacaacggcggtgagaagggctgcacatcacctatccgcattgcagaccagcgatggccattggcctgctcaaattgcaggtcctctgtttttccttccccccttggttttttgtatgtacattacaggacatcttgattcggtattcccagaagagtatcgcaaagagattcttcgttacatatactatcaccagaatgaagatggagggtgggggctacacatagagggtcacagcaccatgttttgtactgcactcaactatatatgcatgcgaattcttggagaagggcccgatgggggtcaagacaatgcttgtgctagagcaagaaagtggattcatgatcatggtggtgtaacacatataccttcatggggaaaaacttggctttcgatacttggtgtatttgattggtgcggaagcaacccaatgccccctgagttttggatcctgccttcatttcttcctatgcatccagctaaaatgtggtgttattgccgattggtatacatgcctatgtcttacttgtatgggaaaaggtttgtgggtccaatcacaccactcattctacagttgagagaagaactctttactgaaccttatgaaaaagttaattggaagaaagcacgtcaccaatgtgcaaaggaagatctttactatccccatcctttgttacaagatctgatatgggatagtttatacttattcactgagccacttctgactcgttggcctttcaacaagctggttagagaaaaggcccttcaagtaacaatgaagcatatccactatgaagatgagactagtcgatacataaccattgggtgtgtggaaaaggttttatgtatgcttgcttgttgggtggaagatccaaatggagatgctttcaagaagcatcttgcaagggtcccggattacttatgggtttcggaagatggaatgaccatgcagagttttggtagccaagaatgggatgctggttttgccgttcaagctttgcttgccactaacctagtcgaggaaattgctcccacacttgccaaaggacatgatttcatcaagaaatctcaggttagggacaacccttcaggagattttaagagcatgtatcgtcatatttctaaaggctcatggaccttctccgatcaagaccatggatggcaagtttctgattgcacagcagaaggtttgaagtgttgtctacttttatcaatgttgcctccagagattgtgggggaaaagatggaacctgaaaggttatatgattcagtcaatgtcctattgtcgcttcagagtaaaaagggtggtttatcagcgtgggagccagcaggagctcaagaatggttagaactactcaatcccacggaattttttgcggacattgtagttgagcatgaatacgttgagtgcactggatcagcaattcaggcactagttctgttcaagaagctatatccagggcataggaagaaagagatagagaatttcattgccaatgcagttcgattccttgaagatacacaaacagctgatggttcatggtatggaaattggggagtttgcttcacttacggttcttggttcgcacttggtggtcttgcagctgctggcaagacttttgccaattgtgctgctattcgcaaagctgttaaatttcttctcacaacacagagagaggatggtggatggggggagagctatctttcaagcccaaaaaagatatatgtacctcttgaaggaagtcgatcaaatgttgtacatactgcatgggcccttatgggtttaattcatgctggccaggcagagagagaccctgctcctcttcaccgagctgcaaaattgatcatcaattcccaattggaagagggtgattggccccaacaggaaatcacgggagtattcatgaaaaattgtatgctgcattacccaatgtatagagatatttatccaatgtgggctctagctgagtatcgtagacgggttccattgccttccactccagcttaa

SEQ ID NO: 9 - DNA, 2289 bp, synthetic construct
  source 1..2289: mol_type = other DNA
atgtggagattaaagattgcggaaggcgggaaggacccctatatatattcaacaaacaattttgttggaaggcagacctgggagtatgatcccgggggcggtagcgctgaagaaagagcgcaggtagatgcagctagactacacttctacaacaatcgtttccaggtcaaaccctgtggtgatttgctgtggagattccaaatactaagagaaaataatttcaagcagacgattgctagtgttaaaataggagacggcgaggagattacatacgaaaaggcgacaacagcggtgagaagggctgcgcatcacctaagtgccttgcaaacttccgatggacactggcctgcccaaatcgccgggcctctatttttcctgcctcctttagtcttttgtatgtacataactggtcacctggacagcgtgttcccggaggagtaccgtaaagaaatcttaagatatatctactatcaccaaaacgaagatggggggtggggcttgcacattgagggtcactccacgatgttctgcaccgctttgaattatatatgtatgcgtatccttggtgagggcccagacggaggacaggacaacgcctgtgcaagagccagaaaatggatccatgaccacgggggggtaactcacattccgagctggggaaagacatggctatctatcctgggtgtatttgattggtgcgggagtaaccccatgccacctgagttctggattctgccctccttcctgccaatgcatcccgctaagatgtggtgttattgtaggcttgtctatatgccaatgagctacctttatgggaagcgtttcgtaggacccataacacctttaattctacaactgagagaggaactttttacagagccgtacgaaaaagtaaattggaagaaggctagacaccagtgtgccaaggaagatctgtactacccgcatcctttgctgcaggaccttatctgggattctttgtacctgtttacagagccacttctgaccagatggcccttcaacaagctggtgagagaaaaggcgttgcaggtgacaatgaagcatatccactatgaggacgagacaagtaggtacataacgatcggttgtgttgaaaaagtcttatgtatgctggcatgctgggttgaggatcctaacggtgacgccttcaagaagcatctggcgagagtgcctgattacctatgggtgtctgaggacggtatgactatgcaaagcttcgggtcccaggagtgggatgcgggatttgcagttcaagctctgttagccactaatttagtggaggaaattgcacccacacttgcgaagggacacgactttattaaaaaatcacaagtcagggataatcctagtggcgatttcaagagtatgtaccgtcacatctctaagggatcttggacgtttagcgaccaggaccacgggtggcaagtgtcagactgcacggccgagggtctaaagtgctgccttttattgtcaatgctgccaccggagatcgttggtgagaagatggagcctgagagattgtatgacagtgtcaatgttctgttgtccctacaatcaaagaaaggcggcctaagtgcttgggagccagcaggtgcacaggaatggttagaacttctaaaccctacagaattcttcgcagacatcgttgtagaacacgagtacgtcgaatgcactggcagtgcaatacaagctcttgttttattcaagaagctataccctggacataggaagaaagagatagagaacttcatcgctaatgccgttcgtttccttgaggatacgcaaactgctgacgggagttggtacggtaattggggcgtctgtttcacgtacggctcctggtttgcgcttggcggtctagccgcagctggtaaaaccttcgccaactgcgcagccattcgtaaagctgtaaaattcttacttactacacaaagagaggatgggggctggggagagagctatttgagttcaccgaaaaaaatttacgtacccttagagggcagtcgttctaatgtcgtgcacacggcatgggctctgatgggacttatccatgccgggcaggccgagcgtgaccccgctccattacacagggcggccaaattaatcattaattcccagcttgaagaaggcgactggccgcagcaagaaatcacaggggtctttatgaagaactgtatgttacactacccaatgtacagggacatctaccctatgtgggcgttggccgaataccgtaggagggttccgcttccgtcaacgccagcttaa

SEQ ID NO: 10 - protein, 760 aa, Gypsophila vaccaria
MWRLKIAEGANDPYLYSTNNFVGRQTWEFDTDYGTPEAIKEVEEARQDFYKNRFQVKPCGDLLWRFQFLREKNFKQTIPQVKLGDGEEVTYEAATATVKRAVNYLAAIQAEDGHWPAEIAGPQFFLPPLVFCLYITGHLNSVFNVHHREEILRSIYYHQNEDGGWGLHIEGHSTMFCTALNYICLRMLGVGPDEGDDNACPRARKWILDHGSVTHIPSWGKTWLSILGLFDWSGSNPMPPEFWILPSFMPMYPAKMWCYCRMVYMPMSYLYGKRFVGPITPLIKQLREELFNEPYEDIKWKKVRHFCAQEDLYYPHPLIQDLMWDSLYLFTEPLLTRWPFNGLIRKKALQVTMDHIHYEDENSRYLTIGCVEKVLCMLACWVEDPNGVCYKKHLARVPDYVWIAEDGLKMQSFGSQQWDCGFAVQALLASNLSLDEIGPALKKGHYFIKESQVKDNPSGDFKSMHRHISKGSWTFSDQDHGWQVSDCTAEGLKCCLVLSTMPPEIVGEKMDPERLYDSVNILLSLQSENGGLSAWEPAGAQAWLELLNPTEFFADIVIEHEYVECTGSAIQALVLFKKLYPGHRKKEIENFILKASKYLEDTQYPNGSWYGNWGVCFTYGTWFALGGLTAAGRTFSNCAAIRKGVEFLLKSQKEDGGWGESYISCPKKDFVPLEGPSNLTQTAWALMGLIYTRQMERDPTPLHRAAKLLINSQLESGDFPQQEITGVFMKNCMLHYPMYRSIYPMWALAEYRKHVPLRLN

SEQ ID NO: 11 - DNA, 2511 bp, Gypsophila vaccaria
  source 1..2511: mol_type = genomic DNA
cgattttgtaacacgtacgtacaacacgtaaattattgttacgccttattattataaggtctaaattaagtgattataacataatgtggaggttaaagatagccgaaggtgcaaatgacccgtacttgtatagcacgaataatttcgtaggacgtcaaacttgggaatttgatactgattacggtactcctgaagcgataaaagaagttgaagaagctcgtcaagatttttataaaaatcggtttcaagttaagccttgtggcgacctgttatggcgttttcagttcctaagagagaaaaacttcaagcaaacaataccgcaagtgaagttgggcgatggggaggaggtcacctacgaggccgccacagcgacggtcaagagggccgtcaactacctcgccgccatacaagccgaagacggtcactggcccgctgaaatcgcgggccctcagtttttcctacctcctttggtgttttgcttgtacatcactggacatctcaactcggttttcaatgttcaccaccgcgaagaaattcttcgtagcatttactatcatcagaatgaggatggagggtgggggttgcacattgaaggacacagcaccatgttctgtacggcgttgaactacatatgtctgcggatgctaggagtcggtcctgatgaaggagacgacaatgcgtgccctagggctcgtaaatggatccttgaccatggtagtgtcactcatattccttcttggggaaagacttggctctctatacttggcctgttcgattggtctggaagcaaccctatgccaccggagttttggattcttccttctttcatgcctatgtatccagcaaaaatgtggtgttactgtcggatggtgtacatgccgatgtcatacttatacgggaagaggttcgttggtccgattacacctctaatcaaacagcttagagaggagcttttcaatgagccatatgaggacatcaaatggaaaaaagtccgtcatttctgtgcacaggaggatctgtattacccacatccattgattcaagatttaatgtgggatagtctgtacctattcaccgagcctcttcttactcgctggccattcaacggtctgatacggaagaaggccttacaagtgactatggatcatatacattacgaagatgagaacagtcgatacttaaccataggatgcgttgagaaggttttgtgtatgttggcgtgctgggttgaagacccgaatggtgtttgttacaaaaaacatcttgctagagtacccgattatgtatggattgccgaggatggacttaaaatgcagagttttgggagtcaacaatgggactgtggttttgctgtgcaagctttgctagcttcaaacttgagtctcgacgagattggacccgcccttaagaaaggccactactttatcaaagagtctcaggtgaaagacaatccctctggtgatttcaagagtatgcaccgccatatctcgaaggggtcatggacattttctgatcaagatcatggctggcaggtatcagactgcaccgcagaaggccttaagtgttgcctggtcttgtcgacaatgccaccggaaattgttggagaaaagatggaccctgagagactctatgactctgtcaatatcctgctttctctacagagtgaaaacggagggctatctgcttgggagccggctggagctcaagcttggttagagcttctcaacccaacggagttctttgccgacattgtcatcgagcatgagtatgttgaatgtactggttcagcaattcaagctctggtattattcaagaaattgtaccccggtcacagaaagaaagagatcgaaaatttcattttgaaggcatcaaaatacctcgaggacacccaatatccaaacggttcttggtatggaaattggggtgtttgtttcacctatgggacgtggtttgcgctaggaggactcacagccgctggcagaaccttctcgaattgcgctgctattcggaaaggtgttgagtttctactcaaatcgcagaaggaggatggcgggtggggtgaaagctacatttcatgcccgaagaaggactttgtgccactagaaggaccatccaatttaactcaaactgcgtgggcgttgatgggtctaatttatacaagacagatggagagggatcctacgccactacaccgggcagccaaacttctgatcaattcgcaactcgaaagtggtgatttccctcaacaggaaataacaggagtgttcatgaagaattgcatgttacactatccaatgtataggagtatttacccaatgtgggctcttgcagaatataggaagcatgttcctttgaggcttaattaagcacccaatttagcaagcttttagtaataataaactaataatcgccataatctcgtctttaattatcttccatttactttttttcggttacaatttagccgcttctactttcaaccttctgtattactaccatgtaattctcggg

SEQ ID NO: 12 - DNA, 2283 bp, synthetic construct
  source 1..2283: mol_type = other DNA
atgtggagacttaaaatcgcggagggggccaatgacccatatctgtactcaacgaataactttgtcggtcgtcagacttgggagtttgatactgattacggtactccagaagctataaaagaggttgaggaggcaagacaggacttctacaaaaatcgttttcaggtgaaaccttgcggtgatctgctttggaggtttcagttcctgcgtgaaaaaaattttaaacaaaccataccccaggtcaaattaggagatggtgaagaggtcacatacgaggctgcaacggccacagtcaaacgtgctgtaaactacctagctgcgatccaggccgaagatggccactggccagccgaaatagcaggtcctcagttctttttaccaccgttagtattctgtttatacataacgggacacctgaattcagtcttcaacgttcaccatagagaggagattttgcgtagcatctactaccaccaaaatgaggacggcggatgggggcttcacattgaggggcattccacaatgttctgcaccgcacttaactacatttgcctaaggatgctaggtgtaggtcctgatgagggtgatgacaatgcgtgtccgagggctcgtaagtggatattagaccacggatctgtcacccatattccgtcttggggaaaaacttggctaagtattctgggcctattcgattggagcggtagcaatcccatgccccccgaattttggattttaccgtctttcatgccgatgtaccctgcaaagatgtggtgttattgcagaatggtgtatatgccaatgtcatacctttacggaaagagatttgtgggacctattacgccattaatcaagcaacttcgtgaagaattatttaatgagccgtatgaagatatcaagtggaagaaggtgcgtcatttttgtgctcaagaggatctatattaccctcacccgttaatccaggatctgatgtgggattcactatacctgtttaccgagcccctgttgacaaggtggccttttaacggcttaattagaaaaaaggcactacaggtcacaatggatcatatccactatgaggacgagaattcacgttatctaacgataggatgcgtcgagaaggtgttgtgcatgcttgcatgttgggttgaagatcccaatggggtctgctataaaaaacatctagcaagagtccctgactacgtttggattgccgaagacggactgaagatgcaatcatttggatcccagcaatgggattgtggctttgccgttcaggctctactagcaagcaatttgagccttgatgagatcggaccggcgctgaaaaagggtcattattttatcaaggagagccaagttaaggataacccgtctggtgattttaagtccatgcatagacatatatcaaagggctcttggacattttccgaccaagaccatggttggcaagtatctgattgcactgccgaggggctgaaatgctgcctggtactaagcaccatgccccctgaaatagtgggcgagaaaatggaccccgaacgtttgtatgacagcgttaatatattgctgagtcttcagagtgagaatggagggctatccgcatgggaacctgctggtgcacaagcttggctggagttacttaaccctaccgaattctttgccgatatcgtgatcgagcacgaatacgtcgagtgcactggtagcgcaatccaggcacttgtgctttttaaaaaactttatccgggtcatcgtaaaaaagaaatcgagaacttcatacttaaggcttccaagtatttggaggatacgcaataccccaatgggtcctggtatggaaactggggtgtatgtttcacatacggcacgtggttcgccttgggaggcttaaccgcagcaggtcgtacgttttctaactgcgcagctattcgtaagggggttgagttcctacttaaaagtcagaaagaagacggtgggtggggcgaatcatacatcagttgccctaagaaagacttcgttccactggagggcccatccaatctaactcagactgcatgggcccttatgggcctgatttatacccgtcaaatggaacgtgatccgacgccactacatagagcggccaagcttctgatcaacagccagctagaatctggagattttccacagcaagaaatcaccggcgtcttcatgaaaaattgtatgcttcactatcccatgtaccgttctatttacccaatgtgggccttagccgagtaccgtaagcacgtcccattaaggctaaattaa

SEQ ID NO: 13 - protein, 760 aa, Saponaria vaccaria; identical to SEQ ID NO: 10

SEQ ID NO: 14 - DNA, 2283 bp, Saponaria vaccaria
  source 1..2283: mol_type = genomic DNA
atgtggaggttaaagatagccgaaggtgcaaatgacccgtacttgtatagcacgaataatttcgtaggacgtcaaacttgggaatttgatactgattacggtactcctgaagcgataaaagaagttgaagaagctcgtcaagatttttataaaaatcggtttcaagttaagccttgtggcgacctgttatggcgttttcagttcctaagagagaaaaacttcaagcaaacaataccgcaagtgaagttgggcgatggggaggaggtcacctacgaggccgccacagcgacggtcaagagggccgtcaactacctcgccgccatacaagccgaagacggtcactggcccgctgaaatcgcgggccctcagtttttcctacctcctttggtgttttgcttgtacatcactggacatctcaactcggttttcaatgttcaccaccgcgaagaaattcttcgtagcatttactatcatcagaatgaggatggagggtgggggttgcacattgaaggacacagcaccatgttctgtacggcgttgaactacatatgtctgcggatgctaggagtcggtcctgatgaaggagacgacaatgcgtgccctagggctcgtaaatggatccttgaccatggtagtgtcactcatattccttcttggggaaagacttggctctctatacttggcctgttcgattggtctggaagcaaccctatgccaccggagttttggattcttccttctttcatgcctatgtatccagcaaaaatgtggtgttactgtcggatggtgtacatgccgatgtcatacttatacgggaagaggttcgttggtccgattacacctctaatcaaacagcttagagaggagcttttcaatgagccatatgaggacatcaaatggaaaaaagtccgtcatttctgtgcacaggaggatctgtattacccacatccattgattcaagatttaatgtgggatagtctgtacctattcaccgagcctcttcttactcgctggccattcaacggtctgatacggaagaaggccttacaagtgactatggatcatatacattacgaagatgagaacagtcgatacttaaccataggatgcgttgagaaggttttgtgtatgttggcgtgctgggttgaagacccgaatggtgtttgttacaaaaaacatcttgctagagtacccgattatgtatggattgccgaggatggacttaaaatgcagagttttgggagtcaacaatgggactgtggttttgctgtgcaagctttgctagcttcaaacttgagtctcgacgagattggacccgcccttaagaaaggccactactttatcaaagagtctcaggtgaaagacaatccctctggtgatttcaagagtatgcaccgccatatctcgaaggggtcatggacattttctgatcaagatcatggctggcaggtatcagactgcaccgcagaaggccttaagtgttgcctggtcttgtcgacaatgccaccggaaattgttggagaaaagatggaccctgagagactctatgactctgtcaatatcctgctttctctacagagtgaaaacggagggctatctgcttgggagccggctggagctcaagcttggttagagcttctcaacccaacggagttctttgccgacattgtcatcgagcatgagtatgttgaatgtactggttcagcaattcaagctctggtattattcaagaaattgtaccccggtcacagaaagaaagagatcgaaaatttcattttgaaggcatcaaaatacctcgaggacacccaatatccaaacggttcttggtatggaaattggggtgtttgtttcacctatgggacgtggtttgcgctaggaggactcacagccgctggcagaaccttctcgaattgcgctgctattcggaaaggtgttgagtttctactcaaatcgcagaaggaggatggcgggtggggtgaaagctacatttcatgcccgaagaaggactttgtgccactagaaggaccatccaatttaactcaaactgcgtgggcgttgatgggtctaatttatacaagacagatggagagggatcctacgccactacaccgggcagccaaacttctgatcaattcgcaactcgaaagtggtgatttccctcaacaggaaataacaggagtgttcatgaagaattgcatgttacactatccaatgtataggagtatttacccaatgtgggctcttgcagaatataggaagcatgttcctttgaggcttaattaa

SEQ ID NO: 15 - protein, 758 aa, Quillaja saponaria
MWRLKIAEGGSDPYLFSTNNFVGRQTWEFEPEAGTPEERAEVEAARQNFYNNRYQVKPCDDLLWRYQFLREKNFKQTIPPVKVEDGQEITYEMATTSMQRAARHLSALQASDGHWPAQIAGPLFFMPPLVFCVYITGHLNTVFPSEHRKEILRYMYYHQNEDGGWGLHIEGHSTMFCTALNYICMRILGEGPEGGQDNACARARMWILDHGGVTHIPSWGKTWLSILGLFEWSGSNPMPPEFWILPSFLPMHPAKMWCYCRMVYMPMSYLYGKRFVGPITPLIVQLREEIHTQNYHEINWKSVRHLCAKEDIYYPHPLIQDLIWDSLYILTEPLLTRWPLNKLVRERALQVTMKHIHYEDENSRYITIGCVEKVLCMLACWVDDPNGDAFKKHLARVPDYVWVSEDGITMQSFGSQEWDAGFAVQALLASNLTEELGPALAKGHDFIKQSQVKDNPSGDFKSMYRHISRGSWTFSDQDHGWQVSDCTAEGLKCCLLLSMLPPEIVGEKMEPQRLFDSVNVLLSLQSKKGGLAAWEPAGAQDWLELLNPTEFFADIVVEHEYVECTGSAIQALVLFKKLYPGHRKKEIDSFITNAVRFLENTQTADGSWYGNWGVCFTYGCWFALGGLAAAGKTYNNCPAIRKAVNFLLTTQREDGGWGESYLSSPKKIYVPLEGSRSNVVHTAWAMMGLIHAGQAERDSTPLHRAAKLIINYQLENGDWPQQEITGVFMKNCMLHYPMYRNIYPMWALAEYRRRVPLP

SEQ ID NO: 16 - DNA, 2277 bp, Quillaja saponaria
  source 1..2277: mol_type = genomic DNA
atgtggaggctgaagatagcagaaggtggttccgatccatatctgttcagcacaaacaacttcgtgggtcgccagacatgggagttcgaaccggaggccggcacacctgaggagcgagcagaggtcgaagctgcccgccaaaacttttacaacaaccgttaccaggtcaagccctgtgacgacctcctttggagatatcagttcctgagagagaagaatttcaaacaaacaataccgcctgtcaaggttgaagatggccaagaaattacttatgagatggccacaacctcaatgcagagggcggcccgtcacctatcagccttgcaggccagcgatggccattggccagctcaaattgctggccccttgttcttcatgccacccttggtcttttgtgtgtacattactgggcatcttaatacagtattcccatctgaacatcgcaaagaaatccttcgttacatgtactatcaccagaacgaagatggtgggtggggactgcacatagagggtcacagcaccatgttttgcacagcactcaactacatttgtatgcgtatccttggggaaggaccagaggggggtcaagacaatgcttgtgccagagcacgaatgtggattcttgatcatggtggtgtaacacatattccatcttggggaaagacctggctttcgatacttggtctatttgagtggtctggaagcaatccaatgcctccagagttttggatccttccttcatttcttcctatgcatccagcaaaaatgtggtgctattgccggatggtttacatgcccatgtcttatttatatgggaaaaggtttgttggcccaatcacgcctctcattgttcagttaagagaggaaatacacactcaaaattaccatgaaatcaactggaagtcagtccgccatctatgtgcaaaggaggatatctactatccccatccactcatccaagatttgatttgggacagtttgtacatactaacggagcctcttctcactcgctggcccttgaacaagttggtgcgggagagggctctccaagtaacaatgaagcatatccactatgaagatgaaaatagtcgatacataaccattggatgtgtggaaaaggtgttatgtatgcttgcttgttgggttgatgatccaaatggagatgctttcaagaagcaccttgctcgagtcccagattacgtatgggtctctgaagatggaattactatgcagagttttggtagtcaagaatgggatgctggctttgccgtccaggctctgcttgcttctaatcttaccgaggaacttggccctgctcttgccaaaggacatgacttcataaagcaatctcaggttaaggacaatccttcaggtgacttcaaaagcatgtatcgtcacatttctagaggatcatggaccttctctgaccaagatcatggatggcaagtttctgattgcactgcagaaggtctgaagtgttgcctgcttttgtcgatgttgccaccagaaattgttggtgaaaaaatggaaccacaaaggctatttgattctgtcaatgtgctgctctctctacagagcaaaaaaggtggtttagctgcctgggagccagcaggggcgcaagattggttggaattactcaatcccacagaattttttgcggacattgtcgttgagcatgaatatgttgaatgtactggatcagcaattcaggcattagttttgttcaagaagctgtatccggggcacaggaaaaaagagattgacagtttcattacaaatgctgtccggttccttgagaatacacaaacggcagatggctcttggtatggaaactggggagtttgcttcacctatggttgttggttcgcactgggagggctagcagcagctggcaagacttacaacaactgtcctgcaatacgcaaagctgttaatttcctacttacaacacaaagagaagacggtggttggggagaaagctatctttcaagcccaaaaaagatatatgtacccctggaaggaagccgatcaaatgtggtacatactgcatgggctatgatgggtctaattcatgctgggcaggctgaaagagactcaactcctcttcatcgtgcagcaaagttgatcatcaattatcaactagaaaatggcgattggccgcaacaggaaatcactggagtattcatgaaaaactgcatgttacattaccctatgtacagaaacatctacccaatgtgggctcttgcagaataccggaggcgggttccattgccttaa

SEQ ID NO: 17 - protein, 478 aa, Bupleurum falcatum
MELSITLMLIFSTTIFFIFRNVYNHLISKHKNYPPGSMGLPYIGETLSFARYITKGVPEKFVIERQKKYSTTIFKTSLFGENMVVLGSAEGNKFIFGSEEKYLRVWFPSSVDKVFKKSHKRTSQEEAIRLRKNMVPFLKADLLRSYVPIMDTFMKQHVNSHWNCETLKACPVIKDFTFTLACKLFFSVDNPLELEKLIKLFVNIVNGLLTVPIDLPGTKFRGVIKSVKTIRHALKVLIRQRKVDIREKRATPTQDILSIMLAQAEDENYEMNDEDVANDFLAVLLASYDSANTTLTMIMKYLAEYPEMYDRVFREQMEVAKTKGKDELLNLDDLQKMNYTWNVACEVLRIATPTFGAFREVIADCTYEGYTIPKGWKLYYAPRFTHGSAKYFQDPEKFDPSRFEGDGAPPYTFVPFGGGLRMCPGYKYAKIIVLVFMHNIVTKFKWEKVNPNEKMTVGIVSAPSQGLPLRLHPHKSPS

SEQ ID NO: 18 - DNA, 1437 bp, Bupleurum falcatum
  source 1..1437: mol_type = genomic DNA
atggaactttctatcactctgatgcttattttctcaacaaccatcttctttatatttcgtaatgtgtacaaccatctcatctctaaacacaaaaactatccccctggaagtatgggcttgccttacattggcgaaacacttagtttcgcgagatacatcaccaaaggagtccctgaaaaattcgtaatagaaagacaaaagaaatattcaacaacaatatttaagacctccttgttcggagaaaacatggtggtgttgggcagtgcagagggcaacaaatttatttttggaagcgaggagaagtatttacgagtgtggtttccaagttctgtggacaaagtgttcaaaaaatctcataagagaacgtcgcaggaagaagctattaggttgcgcaaaaacatggtgccatttctcaaagcagatttgttgagaagttatgtaccaataatggacacatttatgaaacaacatgtgaactcgcattggaattgcgagaccttgaaggcttgtcctgtgatcaaggattttacgtttactttagcttgtaaacttttttttagtgtagacaatcctttggagctagagaagttaatcaagctatttgtgaatatagtgaatggcctccttacggtccctattgatctcccggggacaaaatttagaggagttataaagagtgtcaagactattcgccatgcgcttaaagtgttgatcaggcaacgaaaggtggatattagagagaaaagagccacacctacgcaagatatattgtcgataatgctggcacaggctgaggacgagaactatgaaatgaatgatgaagatgtggccaatgactttcttgcagttttgcttgctagttatgattctgccaatactacactcaccatgattatgaaatatcttgctgaatatcccgaaatgtatgatcgagttttcagagaacaaatggaggtggcaaagacgaaaggaaaagatgaattactcaacttggacgacttgcaaaagatgaattatacttggaatgtagcttgtgaagtactgagaattgcaacaccaacgttcggagcattcagagaggttattgcagattgtacatacgaagggtacaccataccaaaaggctggaagctatattatgccccgcgttttacccatggaagtgcaaaatactttcaagatccagagaaatttgatccatcgcgatttgaaggtgatggtgcgcctccttatacattcgttccattcggaggagggctccggatgtgccctggatacaagtatgcaaagattatagtactagtgttcatgcacaatatagttacaaagttcaaatgggagaaagttaaccctaatgagaaaatgacagtaggaatcgtatcagcgccaagtcaaggacttccactgcgtctccatccccacaaatctccatcttaa

SEQ ID NO: 19 - DNA, 1437 bp, synthetic construct
  source 1..1437: mol_type = other DNA
atggagttatcaatcacgctaatgctaattttttcaacaaccatatttttcatcttcaggaatgtatacaatcatcttataagcaaacacaagaattatcctccagggagtatgggcctgccttacatcggtgaaacgcttagctttgcccgttatatcacaaagggcgttccggaaaaatttgtaatcgagagacagaagaaatatagtaccactattttcaaaaccagtctatttggtgagaacatggtagttctgggttctgcagaggggaacaaatttatcttcgggtcagaggaaaagtatcttagggtttggttcccatcttcagtggacaaggtttttaaaaagtcacacaaacgtacgagtcaggaggaggcgattagacttaggaaaaacatggtcccttttctaaaagcagatttgctgagaagttatgttccaatcatggacaccttcatgaagcaacacgtgaattcccactggaattgtgagacattgaaagcgtgccctgtcatcaaagatttcacgttcactcttgcctgcaaactgttcttcagtgttgacaatccgttagaactggagaagttgataaaattgtttgtgaatattgtgaacgggcttttaactgttccgatagaccttccgggcactaagttccgtggggtcataaaatctgttaagacaataaggcatgcactgaaggtgcttatcagacaaagaaaagtggacattcgtgagaaaagagcgacgccgacccaagatattttatccattatgttagcgcaagccgaggacgagaactacgagatgaacgatgaggacgtagctaacgattttttagcggtactacttgccagttacgactccgcaaatacgactcttacaatgattatgaagtatctggcagaataccctgagatgtacgacagagtatttcgtgaacagatggaggttgcgaagaccaaaggaaaagacgagctgctgaatcttgacgacctgcaaaaaatgaattacacatggaacgttgcctgtgaggttttgcgtattgctacaccaacgttcggcgcgttccgtgaggtaatcgctgactgtacttacgaaggctatactatccccaagggttggaagctatattatgcgcccagattcactcatgggagcgctaaatacttccaagaccccgaaaagtttgatccatcccgttttgagggtgatggcgctccgccctacacattcgtcccatttggcggaggattgagaatgtgtcctggctacaaatacgctaaaataattgttcttgtttttatgcacaacatcgtcaccaaatttaagtgggagaaggtgaaccccaatgaaaaaatgacggtaggtattgtaagcgcgccaagtcaagggttaccactacgtctgcacccccataagtcccctagctaa

SEQ ID NO: 20 - protein, 480 aa, Quillaja saponaria
MDPFFIFGLLLLALFLSVSFLLYLSRRAYASLPNPPPGKLGFPVVGESLEFLSTRRKGVPEKFVFDRMAKYCRDVFKTSILGATTAVMCGTAGNKFLFSNEKKHVTGWWPKSVELIFPTSLEKSSNEESIMMKQFLPNFLKPEPLQKYIPVMDIITQRHFNTSWEGRNVVKVFPTAAEFTTLLACRVFLSVEDPIEVAKISEPFEILAAGFLSIPINLPGTKLNKAVKAADQIRDAIVQILKRRRVEIAENKANGMQDIASMLLTTPTNAGFYMTEAHISEKILGMIVGGRDTASTVITFIIKYLAENPEIYNKVYEEQMEVVKSKKPGELLNWEDVQKMKYSWCVACEAMRLAPPVQGGFKVAINDFVYSGFNIRKGWKLYWSAIATHMNPEYFPEPEKFNPSRFEGKGPVPYSFVPFGGGPRMCPGKEYSRLETLVFMHHLVTRYNWEKVYPTEKITVDPMPFPVNGLPIRLIPHKHQ

SEQ ID NO: 21 - DNA, 1443 bp, Quillaja saponaria
  source 1..1443: mol_type = genomic DNA
atggatcctttcttcatttttggcttacttctcttggctctctttctctctgtttcttttcttctctacctttcccgtagagcctatgcttctctccccaaccctccgccggggaagctcggcttccccgtcgtcggcgagagtctcgaatttctctccacccgacgcaaaggtgttcctgagaaattcgtcttcgacagaatggccaaatactgtcgggatgtctttaagacatcaatattgggagcaaccaccgccgtcatgtgcggcaccgccggtaacaaattcttgttctccaacgagaaaaaacacgtcactggttggtggccgaaatctgtagagctgattttcccaacctcacttgagaaatcatccaacgaagaatccatcatgatgaaacaattccttcccaacttcttgaaaccagaacctttgcagaagtacatacccgttatggacataattacccaaagacacttcaatacaagctgggaaggacgcaacgtggtcaaagtgtttcctacggctgccgaattcaccacgttgctggcttgtcgggtattcctcagtgttgaggatcccattgaagtagccaagatttcagagccatttgaaatcttagctgctgggtttctttcaatacccataaatcttccgggtaccaaattaaataaagcggttaaggcagcggatcagattagagacgcaattgtacagattttgaaacggagaagggttgaaattgcggagaataaagcaaatggaatgcaagatatagcgtccatgttgttgacgacaccaactaatgctgggttttatatgaccgaggctcacatttctgagaaaattttgggtatgattgttggtggccgtgatactgctagtactgttatcaccttcatcatcaagtatttggcagagaatcctgaaatttataataaggtctatgaggagcaaatggaagtggtaaagtcaaagaaaccaggtgagttgctgaactgggaagatgtgcagaaaatgaagtactcttggtgcgtagcatgtgaagctatgcgacttgctcctcctgttcaaggtggtttcaaggtggccattaatgactttgtgtattctgggttcaacattcgcaagggttggaagttatattggagtgccattgcaacacacatgaatccagaatatttcccagaacctgagaaattcaacccctcaaggtttgaagggaagggaccagtaccttacagcttcgtacccttcggaggcggacctcggatgtgtcccgggaaagagtattcccggctggaaacacttgttttcatgcatcatttggtgacgaggtacaattgggagaaagtgtatcccacagagaagataacagtggatccaatgccattccctgtcaacggcctccccattcgccttattcctcacaagcaccaatga

SEQ ID NO: 22 - DNA, 1443 bp, synthetic construct
  source 1..1443: mol_type = other DNA
atggacccattctttatattcggtttgttactgctagcgttgttcctttcagtctccttccttttatacttatctcgtcgtgcatacgcttcactgccaaatcctccaccgggtaaacttggttttcctgtagttggggaatctttagaattcctttctacccgtaggaaaggggtgcctgagaagtttgtgttcgataggatggccaaatactgtagagacgtatttaagacatcaattctgggcgcgaccaccgccgtgatgtgtgggactgcgggaaataagtttctgttcagtaatgaaaaaaaacacgtcacaggctggtggcctaagagtgtagaattaattttcccgacttcattagagaaaagctccaacgaggaaagcattatgatgaagcaattccttccaaattttctaaagcccgagccgttacaaaaatacataccggtgatggatataatcacccagaggcattttaatacctcatgggagggtcgtaatgtagtaaaggtattccccacagccgcggaattcactacattgctagcttgtagggtttttttatctgtggaggaccccattgaagtcgctaaaatttccgagccattcgagattttggccgcgggattcctaagcataccgattaatttgccaggaactaagttaaataaggcagtgaaagccgctgatcagataagggacgccatcgtacaaattctaaaaaggcgtagggtcgagattgcggaaaataaagctaacggtatgcaggacattgcctcaatgttgcttactacgccaacaaacgcaggtttctacatgacggaggctcatatttccgagaagattctaggtatgatcgtaggaggccgtgataccgccagcacagttatcacctttattataaaatatctggcagaaaatccggaaatctataacaaagtttacgaggagcagatggaagtcgtgaaatccaagaaaccaggtgaactattaaactgggaagatgttcagaagatgaaatacagttggtgtgtagcatgtgaggctatgaggttagcaccaccggtccaaggggggtttaaagttgccattaatgactttgtttacagtgggtttaacatcaggaagggttggaagctatactggagcgccatcgcgactcatatgaaccccgagtactttccagagccagagaagtttaatccgagcaggttcgaaggaaagggcccggtcccttattcatttgtgccattcggtggcggcccccgtatgtgcccgggaaaggagtacagtagactagagactttggtctttatgcaccatctagtaacaaggtataactgggagaaagtctatccgactgaaaagattacggtagacccgatgccattccctgtcaacggattacctatacgtctgataccacataagcatcagtaa

SEQ ID NO: 23 - protein, 509 aa, synthetic construct
MEHLYLSLVLLFVSSISLSLFFLFYKHKSMDPFFIFGLLLLALFLSVSFLLYLSRRAYASLPNPPPGKLGFPVVGESLEFLSTRRKGVPEKFVFDRMAKYCRDVFKTSILGATTAVMCGTAGNKFLFSNEKKHVTGWWPKSVELIFPTSLEKSSNEESIMMKQFLPNFLKPEPLQKYIPVMDIITQRHFNTSWEGRNVVKVFPTAAEFTTLLACRVFLSVEDPIEVAKISEPFEILAAGFLSIPINLPGTKLNKAVKAADQIRDAIVQILKRRRVEIAENKANGMQDIASMLLTTPTNAGFYMTEAHISEKILGMIVGGRDTASTVITFIIKYLAENPEIYNKVYEEQMEVVKSKKPGELLNWEDVQKMKYSWCVACEAMRLAPPVQGGFKVAINDFVYSGFNIRKGWKLYWSAIATHMNPEYFPEPEKFNPSRFEGKGPVPYSFVPFGGGPRMCPGKEYSRLETLVFMHHLVTRYNWEKVYPTEKITVDPMPFPVNGLPIRLIPHKHQ

SEQ ID NO: 24 - DNA, 1530 bp, synthetic construct
  source 1..1530: mol_type = other DNA
atggagcacttgtatctctcccttgtgctcctgtttgtttcctcaatctccctctccctcttcttcctgttctacaaacacaaatctatggatcctttcttcatttttggcttacttctcttggctctctttctctctgtttcttttcttctctacctttcccgtagagcctatgcttctctccccaaccctccgccggggaagctcggcttccccgtcgtcggcgagagtctcgaatttctctccacccgacgcaaaggtgttcctgagaaattcgtcttcgacagaatggccaaatactgtcgggatgtctttaagacatcaatattgggagcaaccaccgccgtcatgtgcggcaccgccggtaacaaattcttgttctccaacgagaaaaaacacgtcactggttggtggccgaaatctgtagagctgattttcccaacctcacttgagaaatcatccaacgaagaatccatcatgatgaaacaattccttcccaacttcttgaaaccagaacctttgcagaagtacatacccgttatggacataattacccaaagacacttcaatacaagctgggaaggacgcaacgtggtcaaagtgtttcctacggctgccgaattcaccacgttgctggcttgtcgggtattcctcagtgttgaggatcccattgaagtagccaagatttcagagccatttgaaatcttagctgctgggtttctttcaatacccataaatcttccgggtaccaaattaaataaagcggttaaggcagcggatcagattagagacgcaattgtacagattttgaaacggagaagggttgaaattgcggagaataaagcaaatggaatgcaagatatagcgtccatgttgttgacgacaccaactaatgctgggttttatatgaccgaggctcacatttctgagaaaattttgggtatgattgttggtggccgtgatactgctagtactgttatcaccttcatcatcaagtatttggcagagaatcctgaaatttataataaggtctatgaggagcaaatggaagtggtaaagtcaaagaaaccaggtgagttgctgaactgggaagatgtgcagaaaatgaagtactcttggtgcgtagcatgtgaagctatgcgacttgctcctcctgttcaaggtggtttcaaggtggccattaatgactttgtgtattctgggttcaacattcgcaagggttggaagttatattggagtgccattgcaacacacatgaatccagaatatttcccagaacctgagaaattcaacccctcaaggtttgaagggaagggaccagtaccttacagcttcgtacccttcggaggcggacctcggatgtgtcccgggaaagagtattcccggctggaaacacttgttttcatgcatcatttggtgacgaggtacaattgggagaaagtgtatcccacagagaagataacagtggatccaatgccattccctgtcaacggcctccccattcgccttattcctcacaagcaccaatga

SEQ ID NO: 25 - DNA, 1530 bp, synthetic construct
  source 1..1530: mol_type = other DNA
atggaacacttgtatttgagtctggtattgttattcgtcagcagcataagcctgtctctgttttttctgttttataaacacaagagcatggacccattctttatattcggtttgttactgctagcgttgttcctttcagtctccttccttttatacttatctcgtcgtgcatacgcttcactgccaaatcctccaccgggtaaacttggttttcctgtagttggggaatctttagaattcctttctacccgtaggaaaggggtgcctgagaagtttgtgttcgataggatggccaaatactgtagagacgtatttaagacatcaattctgggcgcgaccaccgccgtgatgtgtgggactgcgggaaataagtttctgttcagtaatgaaaaaaaacacgtcacaggctggtggcctaagagtgtagaattaattttcccgacttcattagagaaaagctccaacgaggaaagcattatgatgaagcaattccttccaaattttctaaagcccgagccgttacaaaaatacataccggtgatggatataatcacccagaggcattttaatacctcatgggagggtcgtaatgtagtaaaggtattccccacagccgcggaattcactacattgctagcttgtagggtttttttatctgtggaggaccccattgaagtcgctaaaatttccgagccattcgagattttggccgcgggattcctaagcataccgattaatttgccaggaactaagttaaataaggcagtgaaagccgctgatcagataagggacgccatcgtacaaattctaaaaaggcgtagggtcgagattgcggaaaataaagctaacggtatgcaggacattgcctcaatgttgcttactacgccaacaaacgcaggtttctacatgacggaggctcatatttccgagaagattctaggtatgatcgtaggaggccgtgataccgccagcacagttatcacctttattataaaatatctggcagaaaatccggaaatctataacaaagtttacgaggagcagatggaagtcgtgaaatccaagaaaccaggtgaactattaaactgggaagatgttcagaagatgaaatacagttggtgtgtagcatgtgaggctatgaggttagcaccaccggtccaaggggggtttaaagttgccattaatgactttgtttacagtgggtttaacatcaggaagggttggaagctatactggagcgccatcgcgactcatatgaaccccgagtactttccagagccagagaagtttaatccgagcaggttcgaaggaaagggcccggtcccttattcatttgtgccattcggtggcggcccccgtatgtgcccgggaaaggagtacagtagactagagactttggtctttatgcaccatctagtaacaaggtataactgggagaaagtctatccgactgaaaagattacggtagacccgatgccattccctgtcaacggattacctatacgtctgataccacataagcatcagtaa

SEQ ID NO: 26 - protein, 502 aa, Saponaria vaccaria
MELISLLSGLIVLAIVSLTTFFFLCYDTPTKDGKTLPPGRMGWPFIGESYDFFAAGWKGEPESFIFDRLKKFAKGNLNGQFKTSLFGNKSIVVAGAAANKLLFSNEKKLVTMWWPPSIDKAFPSTAQLSANEEALLMRKFFPSFLIRREALQRYIPIMDDCTRRHFATGAWGPSDRIEAFNVTQDYTFWVACRVFMSIDAQEDPATVDSLFKHFNVLKAGIYSMHIDLPWTNFHHAMKASQAIRSAVEQIAKKRRAELAEGKAFPTQDMLSYMLETPITSAEDSKDGKAKYLNDADIGTKILGLLVGGHDTSSTVIAFFFKFMAENPHVYDAIYKEQMEVAAAKAPGELLNWDDLQKMKYSWCAICEVMRLTPPVQGAFRQAITDFTHNGYLIPKGWKIYWSTHSTHRSPEYFPQPEIFDPTRFEGNGPPAFSFVPFGGGPRMCPGKEYARLQVLTFVHHIVTKFRWERILPNEKIIVSPMPYPEKNLPLRMIARSDTTTVA

SEQ ID NO: 27 - DNA, 1509 bp, Saponaria vaccaria
  source 1..1509: mol_type = genomic DNA
atggagttaattagtttactaagtggcttaattgttcttgctatagtcagcttgactacctttttctttttatgctatgatactcctactaaggacggtaaaactctccctcctggtcgtatgggctggccttttataggcgagtcctacgacttttttgcggccggttggaaaggggagcctgagagctttatttttgaccggttgaagaagtttgctaaggggaatcttaatggtcagtttaagacgagtttgtttgggaataagtcaattgtggtggcgggggctgctgctaacaagcttcttttctcgaatgaaaagaagcttgttaccatgtggtggcccccttctattgacaaggcttttccctccaccgcacagctgagtgccaacgaggaggccttgttgatgaggaagttttttccttcctttttgattaggcgggaggcgctccagcgttacattcctattatggatgattgcacccgtcgccattttgccacgggtgcatggggtccatctgaccgcattgaggcctttaatgtgacccaagactacacgttctgggtcgcctgcagggtcttcatgagcattgacgcccaagaagaccctgcaaccgtagactccctctttaagcacttcaacgtgcttaaagcgggaatctactcgatgcacattgacctcccgtggacgaacttccaccacgccatgaaggcgtcccaggccatcaggagcgcggtggagcaaatcgcaaagaaaagaagggccgaattggccgagggaaaggcgttcccgacacaagacatgttatcttacatgctcgaaacgccaatcacctcagccgaggacagcaaagacgggaaagccaagtatctgaacgacgccgacatcggaacgaagatacttggccttctagttggtggccatgacactagtagcaccgttattgccttctttttcaaatttatggctgaaaatcctcatgtttatgatgctatttacaaagaacaaatggaggtggccgcagccaaagcaccgggggagctactaaattgggacgacctgcaaaaaatgaagtactcgtggtgcgccatttgcgaggtcatgcgcttgacgccccccgtccaaggcgcctttcgccaggctatcactgacttcacccataacggctaccttattcccaagggttggaagatatactggagtacacactcgacacatagaagccccgaatacttcccacaaccggagatattcgatccaacaagattcgaaggcaacgggccaccagcgttctcatttgtgcctttcggagggggaccaagaatgtgcccgggtaaggaatatgcaaggctacaagtgctcacatttgtgcaccacattgtgaccaaattccggtgggaacgtatcctacctaatgaaaaaatcatcgttagcccgatgccttaccctgagaagaacctacctcttcgtatgatcgctcgttctgataccactaccgtggcttaa

SEQ ID NO: 28 - DNA, 1509 bp, synthetic construct
  source 1..1509: mol_type = other DNA
atggaactaatctcactattgtctggcctaatcgtcctggccattgtctcccttactacctttttcttcctgtgttacgatacgcccacgaaggacggcaagacgttacctcccggaagaatggggtggccctttataggggaatcttacgatttctttgctgcaggatggaagggggaaccagaatcattcatcttcgacagacttaaaaagttcgcaaagggcaacctgaacggccagtttaaaacgtctctattcgggaacaaatccatagtggtggctggagccgccgctaacaaacttctttttagcaacgaaaaaaaattagtgacaatgtggtggccaccctccattgataaggctttcccgtctaccgctcaattgtccgcgaacgaggaggctctgttaatgcgtaagttttttccttcatttttgatccgtcgtgaggccttgcaaagatatattccgatcatggatgattgtacacgtaggcacttcgcgacgggtgcgtggggccctagcgatcgtatcgaggctttcaatgttacacaggactataccttctgggttgcctgcagggtttttatgtccatcgatgcacaggaggacccggcgacagtggatagtctatttaaacatttcaatgttttgaaagccggaatttactctatgcatattgatttaccctggactaactttcaccacgctatgaaagcgtcccaggccattagaagcgcagtcgagcagatcgctaaaaaaagaagagcggagctggccgagggtaaggcattccctacacaagacatgttgagttacatgttggagaccccgatcacgagtgcggaggatagtaaagacggcaaagcaaaatatctaaacgatgcagacattggcacgaagatacttgggctgttagtcggaggtcatgacacttctagcacggtgattgcattctttttcaagtttatggccgagaatccacacgtgtatgatgctatctataaagaacagatggaggtggcagccgcgaaggctcctggagagttattgaactgggatgatctgcaaaagatgaaatactcttggtgtgctatttgcgaagtaatgcgtctaacgccacccgttcaaggtgcatttagacaggccatcacggattttactcataatgggtacttaattcccaagggttggaagatatattggagcacgcattccacgcataggtcccccgaatatttcccgcaacctgagatattcgatcccacgagattcgaaggaaacggcccaccggcgtttagctttgttccgttcggaggaggcccgaggatgtgccccggaaaagaatacgctaggcttcaagtccttacattcgttcatcatattgtaaccaagttcagatgggagcgtattctacctaatgagaaaattatcgtctctccgatgccatatccggaaaaaaaccttcctcttagaatgattgctaggtccgatacaactacagttgcctga

SEQ ID NO: 29 - protein, 507 aa, Quillaja saponaria
MWFTVGLVLVFALFIRLYSSLWLKPRATRIKLSNQGIKGPKPAFLLGNVAEMRRFQSKLPKSELKQGQVSHDWASKSLFPFFSLWSQKYGNTFVFSLGNIQVLYVSDHELVKEINQNTSLDLGKPKYLQKERGPLLGQGILTSNGQLWAYQRKIMTPELYKEKIKGMCELMVESVAWLVEEWGTKIQAEGGAADIRIDEDLRSFSGDVISKACFGSCYAGGREIFLRLRALQHQIASKALLMGFPGLKYLPIKSNREIWRLEKEIFQLIMKLAEDRKKEQHERDLLQIIIEGAKSSDLSSEAMAKFIVDNCKNVYLAGHETTAMSAGWTLLLLANHPEWQARVRDEILQVTEGRNPDFDMLHKMKLLTMVIQEALRLYPTVIFMSREALEDINVGNIQVPKGVNIWIPVVNLQRDTTVWGADANEFNPERFANGVNNSCKVPQLYLPFGAGPRICPGINLAMTEIKILLCILLTKFSFSVSPNYRHSPVFKLVLEPENGINVIMKKL

SEQ ID NO: 30 - DNA, 1524 bp, Quillaja saponaria
  source 1..1524: mol_type = genomic DNA
atgtggttcacagtaggattggtcttggttttcgccctattcatacgtctctacagcagtctgtggttgaagcctcgtgcaactcggattaagcttagcaatcaaggaattaaaggtccaaaaccagcatttcttctgggtaatgttgcagagatgagaagatttcaatctaagcttccaaaatctgaactcaaacaaggccaagtttctcatgattgggcttctaaatctctgtttccatttttcagtctttggtcccagaaatacggaaatacgttcgtgttctcattggggaacatacaggtgctctatgtttctgatcatgagttggtgaaagaaattaatcagaatacctctttagatttgggcaaacccaagtacctgcagaaggagcgtggccctttgctgggacaaggtattttgacctccaatggacagctttgggcgtaccagagaaaaatcatgactcctgaactctacaaggagaaaatcaagggcatgtgcgagttgatggtggaatctgtagcttggttggttgaggaatggggaacgaagatccaagctgagggtggggcagcagacattagaatagacgaggatcttagaagcttctctggtgatgtaatttcaaaagcttgttttgggagctgctatgccggagggagggaaatctttcttaggctcagagctcttcaacaccaaattgcttccaaagccttactcatgggcttccctggattaaagtacctgcccattaagagcaacagagagatatggagattggagaaggagatcttccagctgattatgaagctggctgaagatagaaaaaaagaacaacatgagagagacctattacagattataattgagggagctaaaagtagtgatctgagttcggaagcaatggcaaaattcattgtggacaactgcaagaatgtctacttggctggccatgaaactactgcaatgtctgctggttggactttgcttctcttggctaatcatcctgagtggcaagcccgtgtccgtgatgagattttacaagtcaccgagggccgcaatcctgattttgacatgctgcacaagatgaaactgttaacaatggtaattcaggaggcactgcgactctacccaacagtcatattcatgtcaagagaagcattggaagatattaatgttggaaacatccaagttccaaaaggtgttaacatatggatacctgtggtaaatcttcaaagggacacaacggtatggggtgcagacgcaaacgagtttaatcctgaaaggtttgccaatggagttaacaattcatgcaaggttccacaactttacctaccatttggagctggacctcgcatttgtcctggaattaatctggccatgactgagatcaagatacttctgtgtatcctgctcaccaagttttcgttttcagtttcacccaactatcgccactcaccggtgtttaaattggtgcttgagcctgaaaatggaatcaatgtcatcatgaagaagctctaa

SEQ ID NO: 31 - DNA, 1524 bp, synthetic construct
  source 1..1524: mol_type = other DNA
atgtggtttactgtaggcctggttctggtttttgctcttttcatcagactatattcaagcctgtggctgaagccccgtgctacgcgtatcaaattgagcaaccaaggcattaagggtcccaagcctgcgttcttgttaggcaatgtggcagagatgcgtagattccaaagcaaactaccgaagtctgaactgaaacaaggtcaagtatctcacgactgggccagcaagagtctgttcccttttttcagcctatggtctcaaaagtacggtaacacgtttgtctttagcttgggtaacatccaagtactgtacgtatctgaccatgaattagtcaaggagattaaccagaatacgagcttggatttaggaaaacccaaataccttcaaaaggaacgtggtccgttgctaggccaagggatcttaacgtcaaacgggcagctgtgggcgtatcagagaaaaatcatgacgcctgagttatataaagaaaagataaagggcatgtgcgaattgatggtggagtctgtagcatggctagtggaagagtggggtactaaaatacaagcagaaggtggtgctgcagatatacgtattgacgaagacttgcgtagtttttctggagatgttattagtaaggcttgttttggctcttgctatgccgggggaagggagatctttcttaggctaagagccctgcaacatcagattgcttctaaagctctgctaatgggattccctggtcttaaatacttgccaatcaagtccaaccgtgagatctggaggcttgaaaaggagatcttccagctaattatgaaactagctgaggatagaaagaaagaacagcatgaaagggatctattgcaaataattattgaaggtgctaagtcttctgatctgagtagcgaagctatggccaaattcatagttgataactgcaagaatgtttatctagcggggcatgagacaacagcaatgtcagcgggctggaccttacttttgttagccaaccatccagaatggcaggcccgtgtcagggatgaaatcctgcaagtaactgaggggagaaatccggactttgatatgttgcataagatgaaactgctaacgatggttatacaggaggctttgaggctgtaccccacggtgatatttatgagccgtgaagctttggaggatatcaacgtggggaatattcaggtacccaagggtgtaaatatttggatccccgttgtgaatctgcagagggatacaactgtatggggtgccgatgcgaatgagttcaacccagaaaggtttgcaaatggggttaacaatagctgtaaagttcctcaattgtacctaccatttggagcaggaccgaggatatgccccggaattaatttagccatgaccgaaataaagatacttttatgtatattactaaccaagttttctttctccgtcagtcctaactacagacacagtccggtttttaagttggtccttgagcctgagaacggaattaacgtcattatgaaaaaattataa

SEQ ID NO: 32 - protein, 524 aa, Saponaria vaccaria
MKAEYVPYIVSSLACIVILRWVMNTMKWLWFEPKRLEKCLRKQGFQGTSYKFIFGDMKESTLLRNEALKMAMPMPFDNHYFPRVNPFVHQLLNKFGTNCFMWMGPVPTLQIGEPELVKEVFNRMHEFQKPKTNPLSALLATGLISYEGDKWTKHRRLINPAFYVEKLKLMMPAFRESILEVVKEWEKRVDEEGSAEIDVWPYLTKLTRDGIARAAFGTIYGDERRIFELLSLQKELVLDLLKYSYIPGYIYLPTKGNKKMKAVNNEIQRLLQNVVGNRKKAMEAGEAAKDDLLGLLMDSNYKESRLEGEGKNKKLIMSSQDIIDECKLFFLAGHETTAVLLVWTMVLLSKHQDWQTRAREEVLATFGMKEPADYDALNRLKTVAMILNEVLRLYPPVVTTNRKLFKGEVKIGNLVIPAGVSISLLTHQANRDPKVWGEDACEFRPDRFAEGMAKATKGSVAFFPFGWGPRICIGQNFALAEAKMTIAMILQRFTFDFSPTYTHAPSGLITLHPQYGAPLVLHRR

SEQ ID NO: 33 - DNA, 1575 bp, Saponaria vaccaria
  source 1..1575: mol_type = genomic DNA
atgaaggcggagtacgtaccctacatcgtatcatcgcttgcatgcatagtaatactaagatgggtgatgaatacaatgaagtggttgtggttcgaaccgaagaggttggaaaaatgccttagaaaacaagggtttcaaggaacttcatacaaatttatatttggcgatatgaaggaaagtactttgttgagaaatgaagccttgaaaatggctatgcctatgccctttgataatcactactttcctcgtgttaatccttttgttcatcaacttctcaacaaatttggtacgaattgttttatgtggatggggccagttccaacgttacaaatcggagaacccgaattagtgaaggaagtatttaatcggatgcacgagtttcagaagcctaaaactaaccctttatcggcgttactcgccactggactcattagctacgaaggcgacaaatggaccaagcatcgccgccttatcaaccctgctttctatgtcgagaagcttaagctcatgatgcctgcatttcgagagagcatattggaggtggtgaaagagtgggagaaaagagtcgacgaagaaggtagtgctgaaatcgacgtatggccatatcttactaaattgacccgcgatggtatcgcaagggctgcatttggcaccatttatggggacgaaagaaggatattcgagctgctctcgcttcagaaagaacttgtcttagatctacttaagtattcttatatccctggttatatatatttgccaacaaaaggaaacaagaagatgaaagcagtgaacaatgagatacaaagactactccaaaacgtggttggaaacaggaagaaggcgatggaagccggagaagccgcaaaggacgatctgttggggttactaatggattccaattacaaggagagcaggctcgaaggcgaggggaaaaacaagaaattaataatgagttcccaagatatcattgatgagtgtaagctcttcttcttagcagggcacgaaacgactgcagtgttacttgtgtggactatggttttgttgagcaagcaccaagactggcaaactcgtgctcgtgaagaagttttggctactttcgggatgaaggaacctgctgattatgatgccttgaaccgtctcaagacggttgcaatgatactaaatgaggtgctgagattgtacccgccggttgttacaactaaccgaaagttatttaagggcgaagtaaagattggcaacttggtcataccagccggtgttagtatctcactattaacccaccaagcaaaccgggacccgaaagtttggggtgaggatgcgtgtgagttccgacctgatagatttgcagaagggatggcgaaggcgactaagggcagtgttgcattcttcccctttggttggggtcctaggatttgcattggccaaaatttcgcgctcgctgaggcgaagatgacgattgctatgatattgcaacgcttcacttttgacttttcaccgacgtacactcatgctccgtctggccttattactttgcacccacaatatggtgctcctctcgtgcttcataggcgttaa

SEQ ID NO: 34 - DNA, 1575 bp, synthetic construct
  source 1..1575: mol_type = other DNA
atgaaagcagagtatgtaccatatattgtgagtagcctagcatgcatcgttatattgaggtgggtgatgaacactatgaaatggctttggttcgagcccaagagactagagaaatgtctgaggaaacagggtttccaggggacatcatacaaatttatatttggagacatgaaggagagcactctgcttagaaacgaggcactgaaaatggcgatgcctatgcctttcgataaccattacttcccgcgtgtgaatccctttgtccatcaactgttaaataaattcggaacaaattgttttatgtggatgggcccggtcccgacactgcagattggggaaccagaactagttaaggaggtttttaataggatgcacgagttccagaaaccgaaaactaatccattaagcgccttgttggcgacggggctaatatcttatgagggagataaatggaccaaacatcgtagattaataaatcctgcgttttatgttgagaaattaaaacttatgatgccggccttcagagaatccattcttgaggtagttaaggaatgggagaagagagtcgatgaggaaggtagtgctgagatcgatgtgtggccctaccttacgaagctaacgagggacggcatagctcgtgcagccttcggaactatatacggagatgagcgtcgtatattcgagctgctatcattacaaaaggagttggtcctggacttgctgaaatatagttatatacctggctatatttatctgccgacgaagggcaacaagaaaatgaaggcggtcaacaatgaaatccaaaggctattacagaacgtagtgggtaacagaaaaaaagctatggaggccggggaggcagcaaaggatgatctactgggcttattgatggacagtaattacaaagaaagtcgtctggaaggagagggcaagaacaagaagcttataatgagttcccaggatataatagacgaatgcaagctgtttttcttggcgggccatgagacgacggctgtattgttagtttggactatggtgctgttaagtaagcatcaagattggcaaacacgtgcaagggaagaagtcttggctacattcgggatgaaggagcccgcggattatgatgcgttgaatagattaaagaccgttgcaatgattcttaacgaagtcttgaggctttatccgccggttgtcaccacgaacagaaaactgtttaaaggggaggtaaaaattggcaatttggtcatcccagcgggtgtctctatatccctacttacacatcaggccaatcgtgaccctaaagtatggggtgaagatgcgtgtgagttccgtccagataggttcgcggaaggcatggctaaagcgaccaaggggtcagtggcgttttttccttttggatggggcccccgtatatgtataggacaaaattttgccttagctgaggccaagatgacgatagccatgatactgcagaggttcacatttgacttctcccccacctatacccatgctccgagtgggctaattaccttgcatcctcagtacggtgccccgctagtgcttcatagaaggtaa

SEQ ID NO: 35 - protein, 523 aa, Saponaria vaccaria
MMEYVPYAIASIACIVILRCVLNAAKWLWFEPKRLEKCLRKQGLKGNSYKFLFGDMKESSMLRNEALKKPMSMPFDNDYFPRINPFVHQLLNKYGNNCILWMGPVPTINIGEPELVREAFNRMHEFQKPKTNPLSALLATGLVSYEGDKWAKHRRLINPAFHVEKLKLMIPAFRESIVEVVKQWEQKVSENGYAEIDVWPSLTSLTGDVISRAAFGSVYGDGRRIFELLALQKELVLSLLKFSYIPGYTYLPTEGNKKMKAVNNEIQRLLENVIQNRKKAMEAGEPAKDDLLGLLMDSNYKESMLEGGGKNKKLIMSFQDLIDECKLFFLAGHETTAVLLVWTLILLSKHQDWQTRAREEVLATFEMREPTDYDALNRLKIVTMILNEVLRLYPPVVTTNRKLFKSEAKIGNLVIPPGVGISLLTIQANRDPKVWGEDASEFRPDRFAEGLVKATKGNVAFFPFGWGPRICIGQNFALTESKMAIAMILQRFTFELSPSYTHAPSGLITLNPQYGAPLMFRRR

SEQ ID NO: 36 - DNA, 1572 bp, Saponaria vaccaria
  source 1..1572: mol_type = genomic DNA
atgatggagtacgtaccgtacgcaatagcgtcgatagcatgcatagtaatactaagatgtgtgctgaatgcagcgaaatggttgtggttcgaaccgaagcggttagagaaatgccttagaaaacaaggacttaaagggaactcgtacaagtttttatttggagatatgaaggaaagttctatgctacgtaatgaggccttaaagaagcccatgtctatgccctttgataatgactactttcctcgtattaacccctttgttcatcaacttcttaataaatatggtaacaattgtatcttgtggatgggacctgttccaactataaatatcggagaacctgagctagtaagggaagctttcaatcggatgcacgagtttcagaagcccaaaactaaccccctgagtgctttgctcgccactggacttgttagctacgaaggtgacaaatgggccaagcaccgccgccttatcaatcctgctttccatgtcgagaagctcaagcttatgattcctgcgttccgtgagagcattgtggaggtggtcaaacaatgggagcagaaagtatctgaaaacggttatgctgaaatagatgtatggccgtctcttactagtttgactggtgatgttatctcaagggctgcgtttggcagcgtctatggcgatggaagaaggattttcgaacttctggctcttcagaaagaactcgttctaagtctcctcaagttttcttacatccctggatatacatatctgccaacagagggaaacaagaagatgaaggcagtgaacaatgagatacaacgactactcgaaaacgtgattcaaaacaggaagaaggcgatggaagccggagaaccagcgaaggatgatctgttggggttacttatggattccaattacaaggagagtatgcttgaaggcggtgggaagaacaaaaagttaatcatgagtttccaagatcttatcgacgagtgtaagctcttcttcttagctgggcatgagaccaccgccgtcttactcgtctggactttgattttgctgagtaagcaccaagactggcaaactcgagctcgtgaagaagttttggctacttttgaaatgagggaacctactgattatgatgccttgaaccgtctcaagattgttacaatgatactgaacgaggtgctgagattgtacccaccagttgtcacaaccaaccgaaaactattcaagagcgaagcaaagattggaaacttggtgataccacctggtgtaggaatctcattgttaaccatccaagcaaaccgcgaccccaaagtttggggggaggacgcgagtgagttccgacctgatagattcgcggaagggctagtgaaggcgactaagggcaatgttgcattcttcccctttggttggggtcctaggatttgcattggccaaaattttgcgctcactgagtctaagatggcgattgccatgatattacaacgcttcactttcgaactttcaccgtcctacactcatgctccgtcgggccttatcactctcaacccacaatatggtgctcctctcatgttccgtagacgttaa

SEQ ID NO: 37 - DNA, 1572 bp, synthetic construct
  source 1..1572: mol_type = other DNA
atgatggagtatgtaccgtatgccattgcgtccattgcgtgtatagtcattttaaggtgcgtccttaatgctgctaagtggctatggtttgaaccaaagagactagagaaatgtttgagaaaacaaggactaaaggggaattcctacaagtttttgttcggtgacatgaaggagagcagcatgctaaggaatgaagcattgaagaagcccatgagcatgcctttcgataatgactattttcccaggatcaacccctttgtccaccaactactgaataaatatggaaacaactgtattttgtggatgggcccagtccctacgataaatataggcgaaccagaattagttcgtgaggcctttaataggatgcatgagttccagaagccgaagaccaatcccttaagtgcgctgctagctactggactggtcagttacgagggagacaaatgggctaagcaccgtagattaattaatcctgccttccatgtagagaagctaaaacttatgatacctgcattcagggagtcaatcgtggaagtggtcaaacagtgggagcagaaagtgtcagaaaacggctatgccgaaattgatgtatggccttccctaacgtccctgaccggtgatgttatctcaagggctgcctttggctccgtgtatggtgatggcaggaggatcttcgaattgttggcgctgcaaaaagagctagtgttgtctcttctgaagtttagttacatcccgggatatacctacctacccaccgaaggaaacaaaaagatgaaagcagttaacaatgagattcaaagattattggagaatgttattcagaacagaaagaaagctatggaggcaggagaacctgccaaggacgaccttctaggccttctaatggattcaaactacaaggaatctatgttggaaggaggcgggaagaacaaaaaactaatcatgagttttcaagacttaatcgacgagtgtaaactgttttttctggcaggccatgaaaccacagccgtgctattggtgtggaccttgatccttttatctaaacatcaggactggcagacccgtgccagggaagaggtcttggctacttttgaaatgagggagcctacggactatgacgccttaaataggcttaagatcgtaacaatgatcttaaacgaagtgctgaggttgtaccccccggtggtgaccaccaaccgtaagttgttcaaatcagaggctaaaattggcaatctggtcattccccccggggttgggatatcccttttgacgatacaagcaaatcgtgatcctaaggtgtggggtgaggacgctagtgaatttagacccgataggtttgctgagggattggttaaggcgacaaaagggaacgtggcctttttcccatttgggtggggaccaaggatctgcattggccaaaactttgcattgacggaatccaagatggccatcgctatgattctacagagattcacgtttgagctgtctcccagttatacacatgccccgtccggtctaattactttaaatccgcaatacggtgctccgctaatgttcaggagaaggtaa

SEQ ID NO: 38 - protein, 520 aa, Medicago truncatula
MELSWETKSAIILITVTFGLVYAWRVLNWMWLKPKKIEKLLREQGLQGNPYRLLLGDAKDYFVMQKKVQSKPMNLSDDIAPRVAPYIHHAVQTHGKKSFIWFGMKPWVILNEPEQIREVFNKMSEFPKVQYKFMKLITRGLVKLEGEKWSKHRRIINPAFHMEKLKIMTPTFLKSCNDLISNWEKMLSSNGSCEMDVWPSLQSLTSDVIARSSFGSSYEEGRKVFQLQIEQGELIMKNLMKSLIPLWRFLPTADHRKINENEKQIETTLKNIINKREKAIKAGEATENDLLGLLLESNHREIKEHGNVKNMGLSLEEVVGECRLFHVAGQETTSDLLVWTMVLLSRYPDWQERARKEVLEIFGNEKPDFDGLNKLKIMAMILYEVLRLYPPVTGVARKVENDIKLGDLTLYAGMEVYMPIVLIHHDCELWGDDAKIFNPERFSGGISKATNGRFSYFPFGAGPRICIGQNFSLLEAKMAMALILKNFSFELSQTYAHAPSVVLSVQPQHGAHVILRKIKT

SEQ ID NO: 39 - DNA, 1563 bp, Medicago truncatula
  source 1..1563: mol_type = genomic DNA
atggaattatcttgggaaacaaaatcagccataattctcatcactgtgacatttggtttggtatacgcatggagggtattgaattggatgtggctgaagccaaagaagatagagaagcttttaagagaacaaggccttcaagggaacccttatagacttttgcttggagatgcaaaggattattttgtgatgcaaaagaaagttcaatccaaacccatgaatctatctgatgatattgcgccacgtgtcgctccttacattcatcatgctgttcaaactcatgggaaaaagtcttttatttggtttggaatgaaaccatgggtgattctcaatgaacctgaacaaataagagaagtattcaacaagatgtctgagttcccaaaggttcaatataagtttatgaagttaataactcgcggtcttgttaaactagaaggagaaaagtggagcaagcatagaagaataatcaaccctgcgtttcacatggaaaaattgaagattatgacaccaacattcttgaaaagctgcaatgatttgattagcaattgggaaaaaatgttgtcttcaaatggatcatgtgaaatggacgtatggccttcccttcagagcttgacaagtgatgttatcgctcgttcgtcatttggaagtagttatgaagaaggaagaaaagtatttcaacttcaaatagagcaaggtgaacttataatgaaaaatctaatgaaatctttaatccctttatggaggtttttacctaccgctgatcatagaaagataaatgaaaatgaaaaacaaatagaaactactcttaagaatataattaacaagagggaaaaagcaattaaggcaggtgaagccactgagaatgacttattaggtctcctcctagagtcgaaccacagagaaattaaagaacatggaaacgtcaagaatatgggattgagtcttgaagaagtagtcggggaatgcaggttattccatgttgcagggcaagagactacttcagatttgcttgtttggacgatggtgttgttgagtaggtaccctgattggcaagaacgtgcaaggaaggaagtattagagatatttggcaatgaaaaacccgactttgatggactaaataaacttaagattatggccatgattttgtatgaggttttgaggttgtaccctcctgtaaccggcgttgctcgaaaagttgagaatgatataaaacttggagacttgacattatatgctggaatggaggtttacatgccaattgttttgattcaccatgattgtgaactatggggtgatgatgctaagattttcaatcctgagagattttctggtggaatttccaaagcaacaaacggtagattttcatattttccgtttggagcgggtcctagaatctgcattggacaaaacttttccctgttggaagcaaagatggcaatggcattgattttaaagaatttttcatttgaactttctcaaacatatgctcatgctccatctgtggtgctttctgttcagccacaacatggtgctcatgttattctacgcaaaatcaaaacataa

SEQ ID NO: 40 - DNA, 1563 bp, synthetic construct
  source 1..1563: mol_type = other DNA
atggagctaagttgggaaactaaaagtgctatcattcttatcacagtgacttttggtctggtatacgcttggcgtgtgttgaactggatgtggcttaagccgaaaaaaatcgagaaactgttaagggagcagggactgcagggcaacccttataggttgttgttgggcgatgccaaggattattttgtgatgcagaagaaggtgcagtctaagcctatgaatctatctgacgatatagctcctagagtggcgccttacatacatcacgcagtacaaacgcacggcaagaagagtttcatatggttcgggatgaagccctgggttatcttgaacgaaccagagcagatcagggaagtatttaataaaatgtcagaatttcccaaagtccagtacaaatttatgaagcttataacaagagggctagtaaagcttgagggcgaaaaatggagcaaacatagacgtattattaatcccgcttttcacatggagaaactaaagataatgacgccgacctttctgaagtcctgcaatgatcttatctcaaattgggagaaaatgctgagttctaacggaagctgtgaaatggatgtgtggccaagcttgcaatcattaacatcagacgttattgctagaagcagctttgggtcctcatacgaggaagggcgtaaggtcttccagctacagatcgaacagggagaacttataatgaaaaacttaatgaagagcttaatccctttatggagatttctaccgactgctgaccatagaaaaattaacgagaacgagaaacaaatagagaccacacttaaaaatatcatcaacaagcgtgaaaaggcgattaaggcaggggaagcaacggaaaatgacttactaggtctgcttttggagagtaatcaccgtgagataaaagaacatggtaatgttaaaaatatgggcttgtcccttgaagaggtggtgggcgaatgtcgtctattccatgttgctggccaggaaactacatctgacttattggtatggactatggttctgttatccagataccctgactggcaagagagggcgaggaaagaggtgctagaaatttttggcaacgagaagccagatttcgatggattgaacaagttgaaaataatggcgatgattctttatgaggtactaaggctataccctcccgttactggtgttgctaggaaagtagagaacgacataaagcttggggatttgacgttatacgctgggatggaggtctatatgccaattgttcttatacatcacgattgcgagttatggggcgatgacgctaagatattcaatccagagaggttttccggaggcatatctaaagctacgaacggcagatttagttattttccttttggagctggaccgcgtatctgcattgggcaaaattttagccttcttgaggccaaaatggccatggctctgatactaaagaacttctcctttgaattgagtcaaacctatgctcacgcccctagcgtcgttcttagcgtccaaccacagcacggtgctcatgtaattttaagaaagattaaaacttaa

SEQ ID NO: 41 - protein, 480 aa, Quillaja saponaria
MEHLYLSLVLLFVSSISLSLFFLFYKHKSMFTGANLPPGKIGYPLIGESLEFLSTGWKGHPEKFIFDRMSKYSSQIFKTSILGEPTAVFPGAVCNKFLFSNENKLVNAWWPASVDKIFPSSLQTSSKEEAKKMRKLLPQFLKPEALHRYIGIMDSIAQRHFADSWENKNQVIVFPLAKRYTFWLACRLFISVEDPTHVSRFADPFQLLAAGIISIPIDLPGTPFRKAINASQFIRKELLAIIRQRKIDLGEGKASPTQDILSHMLLTCDENGQYMNELDIADKILGLLVGGHDTASAACTFIVKFLAELPHIYEQVYKEQMEIAKSKVPGELLNWEDIQKMKYSWNVACEVMRLAPPLQGAFREAITDFVFNGFSIPKGWKLYWSANSTHKSPDYFPEPDKFDPTRFEGNGPAPYTFVPFGGGPRMCPGKEYARLEILVFMHNLVKRFKWEKLVPDEKIVVDPMPIPAKGLPVRLYPHKA

SEQ ID NO: 42 - DNA, 1443 bp, Quillaja saponaria
  source 1..1443: mol_type = genomic DNA
atggagcacttgtatctctcccttgtgctcctgtttgtttcctcaatctccctctccctcttcttcctgttctacaaacacaaatctatgttcaccggggccaacctaccacctggtaaaatcggttacccattgatcggagagagcttggagttcttgtccacgggatggaagggccacccggagaaattcatcttcgatcgcatgagcaagtactcatcccaaatcttcaagacctcgattttaggggaaccaacggcggtgttcccgggagccgtatgcaacaagttcctcttctccaacgagaacaagctggtgaatgcatggtggcctgcctccgtggacaagatctttccttcctcactccagacatcctccaaagaagaggccaagaagatgaggaagttgcttcctcagtttctcaagcccgaagctctgcaccgctacattggtattatggattctattgcccagagacactttgccgatagctgggaaaacaaaaaccaagtcattgtctttcctctagcaaagaggtatactttctggctggcttgccgtttgttcattagcgtcgaggatccgacccacgtatccagatttgctgacccgttccaacttttggccgccggaatcatatcaatcccaatcgacttgccagggacaccgttccgcaaggcaatcaatgcgtcccagttcatcaggaaggaattgttggccatcatcaggcagagaaagatcgatttgggtgaagggaaggcatctccgacgcaggacatactgtctcacatgttgctcacatgcgacgagaacggacaatacatgaatgaattggacattgccgacaagattcttggcttgttggtcggcggacatgacactgccagtgccgcttgcactttcattgtcaagttcctcgctgagcttccccacatttatgaacaagtctacaaggagcaaatggagattgcaaaatcaaaagtgccaggagagttgttgaattgggaggacatccaaaagatgaaatattcgtggaacgtagcttgtgaagtgatgagacttgcccctccactccaaggagctttcagggaagccattactgacttcgtcttcaacggtttctccattccaaaaggctggaagttgtactggagcgcaaattccacccacaaaagtccggattatttccctgagcccgacaagttcgacccaactagattcgaaggaaatggacctgcgccttacacctttgttccatttgggggaggacccaggatgtgcccgggcaaagagtatgcccgattggaaatacttgtgttcatgcataacttggtgaagaggttcaagtgggagaaattggttcctgatgaaaagattgtggttgatccaatgcccattccagcaaagggtcttcctgttcgcctttatcctcacaaagcttga

SEQ ID NO: 43 - DNA, 1440 bp, synthetic construct
  source 1..1440: mol_type = other DNA
atggaacacttgtatttgagtctggtattgttattcgtcagcagcataagcctgtctctgttttttctgttttataaacacaagagcatgtttaccggggcgaacttaccgcccggtaaaataggctaccccctgataggtgaatcattggagtttctttcaacgggatggaaggggcaccctgagaaatttatattcgacaggatgagcaaatattcctcccaaatattcaaaacgagtatactgggggaacctacagcggtatttcccggggctgtgtgtaataagttccttttttccaacgagaacaaacttgtcaacgcatggtggcctgcgagcgttgacaaaattttcccgtcttccttagaaacgagctcaaaagaagaagccaagaaaatgaggaaacttttgccccagtttcttaagcctgaggctttgcataggtatatcggaatcatggacagcattgcacagaggcatttcgctgattcctgggaaaacaaaaatcaggttatagttttccccctggcgaagcgttacacgttctggctagcttgcaggttattcatctcagttgaagatccaacacacgtgagccgttttgcggatcctttccagcttctggcagcgggaatcatttccataccgatagacttgccaggcacaccgtttaggaaggcaattaacgcttcccagttcatcaggaaggagctactggcaattatccgtcagaggaagattgacctaggagaaggtaaggcgtcaccgactcaagacatactatcccatatgctattaacgtgtgacgagaatgggcagtacatgaacgagttggacattgcggacaagatattgggattgttggtaggcgggcacgacacagcgtccgccgcatgtactttcgtagtgaagttcttggcagagcttccccatatttatgagcaggtatacaaggaacagatggagatcgctaagagcaaagtcccgggcgaattgcttaactgggaagatatccaaaagatgaaatattcttggaacgtcgcgtgcgaagtaatgagattagcgcccccggtacaaggtgcgtttcgtgaagcaataacagattttgtcttcaatggatttagcattcccaagggttggaaactatactggtctgcaaactcaactcacaaatcccctgattatttccctgaacccgacaaatttgacccaaccagatttgagggaaacgggccagccccatacacattcgttccattcggaggtggtcctagaatgtgcccaggaaaagagtacgctagacttgaaatcctagttttcatgcataacttagtgaaacgttttaagtgggaaaaactggtgcctgatgaaaaaatcgtagtcgatccaatgccgattccagcaaaagggctaccagttaggctttatccacacaaataa

SEQ ID NO: 44 - protein, 483 aa, Saponaria vaccaria
MELFFICGLVLFFSLSLVSLLVLNNHHSARGYRLPPGRMGWPFIGESYEFLANGWKGYPEKFIFSRLAKYKPSQVFKTSILGEKAVVMCGAACNKFLFSNETKLVQAWWPNSVNKIFPSSTQTSSKEEAKKMRKLLPTFFKPEALQRYIPIMDEIAIRHMKDEWEGKEEIQVFPLAKRYTFWLACRIFLSIDDPVHVAKFADPFNDIASGIISVPIDLPGTPFNRGIKASNVVRQELRAIIKQRKMDLADKKASPTQDILSHMMLTPDEDGRFMNELDIADKILGLLIGGHDTASAACTFVVKFLAELPHIYQGVYKEQMEIAKSKAPGERLNWEDIQKMKYSWNVACEVMRLAPPLQGAFREALSDLMYAGFQIPKGWKLYWSANSTHRNPECFPDPETFDPTRFDGSGPAPYTYVPFGGGPRMCPGKEYARLEILVFMHNIVKRFKWEKLIPNETIVVNPMPTPAKGLPVRLHPHSKPVPA

SEQ ID NO: 45 - DNA, 1452 bp, Saponaria vaccaria
  source 1..1452: mol_type = genomic DNA
atggaactgttcttcatttgtgggctagtcctcttcttctcattatctctagtatccctcttggtcctgaacaaccaccattctgctcgggggtacaggctacccccgggcagaatgggatggccgttcataggcgagtcatacgagtttttagccaacgggtggaaagggtacccagagaagttcatattcagtcggctagccaagtacaagcctagccaagtatttaagacgtccatcctaggagaaaaagccgtagtcatgtgtggcgcggcttgtaacaagttcttgttctcgaacgagaccaaattggtacaagcttggtggcctaactccgttaataagatcttcccttcgtcgactcaaacttcttctaaggaagaagctaagaagatgcgcaagcttcttcctaccttcttcaagcccgaggcgcttcaaaggtacatacccataatggacgagatagcgatccgacacatgaaagacgagtgggaggggaaggaggaaatccaagtattcccactagcgaaacgctacacattctggctagcatgcaggatattcctaagcatagacgacccggtacacgtagcaaagttcgcggacccattcaacgacatagcatcagggatcatatcagtcccgatagacctaccggggacgccattcaacagggggatcaaggcctcgaacgtggtgagacaagaactaagagccatcataaagcagaggaagatggacttggctgacaagaaggcgtccccgacacaggatatactgtcacatatgatgctcacccctgacgaggacggccgctttatgaatgaattggacattgctgataagatacttggcttgttgattggtggacatgatactgctagtgctgcttgtacttttgttgttaagtttcttgctgaacttcctcatatttatcagggtgtttacaaagagcaaatggagatagcaaagtcgaaagcacccggagaacgtctaaattgggaggacatacaaaaaatgaagtactcatggaatgtggcatgtgaagtcatgcgattggcaccacctcttcaaggggcttttcgtgaggccctctccgatttgatgtatgccggtttccaaattcccaagggttggaagctgtactggagtgcaaactcaacacataggaacccagagtgcttcccggacccggaaacattcgacccaacaaggttcgacgggagcggtccagcgccatacacgtacgtaccgttcggaggagggccaagaatgtgcccaggaaaagagtatgcaaggctagaaatattggtgttcatgcacaacattgtcaagagatttaaatgggagaaacttattcctaatgaaaccattgttgtcaatcccatgcctacccctgctaaaggcctaccggtccgcctccatcctcattccaaacccgtaccagcttaa

SEQ ID NO: 46 - protein, 479 aa, Medicago truncatula
MEPNFYLSLLLLFVTFISLSLFFIFYKQKSPLNLPPGKMGYPIIGESLEFLSTGWKGHPEKFIFDRMRKYSSELFKTSIVGESTVVCCGAASNKFLFSNENKLVTAWWPDSVNKIFPTTSLDSNLKEESIKMRKLLPQFFKPEALQRYVGVMDVIAQRHFVTHWDNKNEITVYPLAKRYTFLLACRLFMSVEDENHVAKFSDPFQLIAAGIISLPIDLPGTPFNKAIKASNFIRKELIKIIKQRRVDLAEGTASPTQDILSHMLLTSDENGKSMNELNIADKILGLLIGGHDTASVACTFLVKYLGELPHIYDKVYQEQMEIAKSKPAGELLNWDDLKKMKYSWNVACEVMRLSPPLQGGFREAITDFMFNGFSIPKGWKLYWSANSTHKNAECFPMPEKFDPTRFEGNGPAPYTFVPFGGGPRMCPGKEYARLEILVFMHNLVKRFKWEKVIPDEKIIVDPFPIPAKDLPIRLYPHKA

SEQ ID NO: 47 - DNA, 1440 bp, Medicago truncatula
  source 1..1440: mol_type = genomic DNA
atggagcctaatttctatctctcccttctccttctctttgtcactttcatatctctctctctttttttcatattctacaaacagaaatctccattaaatttgccacctggtaaaatgggttacccaatcataggtgaaagccttgagttcttatcaacaggatggaaaggacatcctgaaaaattcattttcgaccgtatgcgtaaatattcctcagaactctttaaaacatcaatcgtaggagaatctacggtggtttgttgcggagcagcaagtaacaagtttttgttttcaaacgagaataaacttgtgactgcatggtggccagatagtgtaaacaaaatcttccctactacttctcttgactctaacttgaaggaagaatccatcaagatgagaaaattgcttccacaattctttaaacccgaagctctacaacgttatgttggtgtcatggatgttattgctcaaagacattttgttactcattgggataataaaaatgaaatcaccgtctaccccttggccaagaggtacacctttttgttagcttgtcggttgttcatgagcgttgaagacgagaatcatgtagcaaaatttagtgatccatttcagttaattgcggccggaatcatatctctaccaattgatttgccaggaacaccattcaacaaagctataaaggcctcaaactttataagaaaggagttgattaagatcataaagcaaaggagggtagatttggcagaagggacagcatcaccaacacaagatatattgtctcacatgttgttgacaagtgatgaaaatggaaagagtatgaatgaacttaatattgctgataagattcttggccttttgatcggaggacatgacactgctagcgtcgcatgcactttccttgtcaaatatctcggcgagttacctcacatttatgataaagtctatcaagagcaaatggaaattgcaaaatcgaaaccagcaggagaattgttgaattgggatgacctgaagaaaatgaaatactcttggaacgtagcttgtgaagtaatgagactttcccctccactccaaggaggtttcagggaagccatcactgactttatgttcaatggattctcaattcctaagggatggaagctttattggagtgcaaattcaacacataagaacgcagaatgttttcccatgccagagaaatttgacccaacaagatttgaaggaaatggaccagctccttatacttttgttccctttggtggaggaccaaggatgtgtcctggaaaagagtatgcaagattagaaatacttgttttcatgcacaatttggtgaaaaggtttaagtgggaaaaggtgattccagatgagaagattattgttgatccattccccatccctgcaaaggatcttccaattcgcctttatccacacaaagcttaa

SEQ ID NO: 48 - DNA, 1440 bp, synthetic construct
  source 1..1440: mol_type = other DNA
atggagcctaatttttatttgtcacttctactattattcgtgacgttcataagcctgtcattattttttatcttctataaacaaaaatctccactgaacctgccgccggggaaaatgggatatcctataatcggagaaagcctagaattcctgtccactggctggaaaggccatcctgagaagttcattttcgatcgtatgagaaagtatagctctgagctatttaagacaagtatagtgggagaaagcacagttgtatgctgtggagccgcttccaacaaatttttgttttctaatgagaacaagctagttactgcgtggtggcccgatagtgtgaacaagatattcccgaccacatccttggattccaatttgaaagaagaatctattaagatgagaaagctactaccccagttttttaagccggaggctctgcaacgttatgtgggcgttatggacgtgatagctcaaagacacttcgtgacccactgggacaataagaatgaaatcacagtttacccacttgctaagaggtatacgttccttttggcatgccgtctttttatgtccgtcgaagacgaaaatcatgtcgctaaattttcagacccttttcaactgattgcagcaggcattatcagtttgccaattgacttacctggtacccccttcaacaaggcgattaaagcctccaacttcatacgtaaggaattaattaagattatcaaacagcgtagggtagaccttgcagaaggcactgcctctccgacgcaggacatactaagtcatatgcttctgacttcagacgagaatgggaagtcaatgaatgaactaaatattgcggataaaatcttaggtctattaataggtggccatgacacagcatcagtggcatgtacgtttttagtaaaatatctaggggagttaccgcatatttacgataaggtttatcaagaacagatggagatcgccaagagcaaaccagcgggtgaattgttaaattgggacgacttaaagaagatgaagtactcctggaatgtagcatgcgaagttatgagactatccccacctctgcaaggaggattcagagaagcaattacggattttatgtttaatggcttcagtatcccgaaagggtggaaactgtattggtctgcgaatagcacccacaaaaacgcggaatgttttcctatgccagagaagtttgatcctactcgttttgaagggaacggaccagctccgtacacctttgtaccattcggtggtgggcctagaatgtgtccaggtaaggaatatgcaagacttgaaatcctggtctttatgcacaatcttgtaaaacgtttcaaatgggagaaggtgatacctgacgaaaagattatagtggacccttttcccattcccgcgaaagaccttcctatacgtctttacccgcataaggcttaa

SEQ ID NO: 49 - protein, 692 aa, Arabidopsis thaliana [sequence omitted]

SEQ ID NO: 50 - DNA, 2079 bp, Arabidopsis thaliana
  source 1..2079: mol_type = genomic DNA
atgacttctgctttgtatgcttccgatttgtttaagcagctcaagtcaattatggggacagattcgttatccgacgatgttgtacttgtgattgcaacgacgtctttggcactagtagctggatttgtggtgttgttatggaagaaaacgacggcggatcggagcggggagctgaagcctttgatgatccctaagtctcttatggctaaggacgaggatgatgatttggatttgggatccgggaagactagagtctctatcttcttcggtacgcagactggaacagctgagggatttgctaaggcattatccgaagaaatcaaagcgagatatgaaaaagcagcagtcaaagtcattgacttggatgactatgctgccgatgatgaccagtatgaagagaaattgaagaaggaaactttggcatttttctgtgttgctacttatggagatggagagcctactgacaatgctgccagattttacaaatggtttacggaggaaaatgaacgggatataaagcttcaacaactagcatatggtgtgtttgctcttggtaatcgccaatatgaacattttaataagatcgggatagttcttgatgaagagttatgtaagaaaggtgcaaagcgtcttattgaagtcggtctaggagatgatgatcagagcattgaggatgattttaatgcctggaaagaatcactatggtctgagctagacaagctcctcaaagacgaggatgataaaagtgtggcaactccttatacagctgttattcctgaataccgggtggtgactcatgatcctcggtttacaactcaaaaatcaatggaatcaaatgtggccaatggaaatactactattgacattcatcatccctgcagagttgatgttgctgtgcagaaggagcttcacacacatgaatctgatcggtcttgcattcatctcgagttcgacatatccaggacgggtattacatatgaaacaggtgaccatgtaggtgtatatgctgaaaatcatgttgaaatagttgaagaagctggaaaattgcttggccactctttagatttagtattttccatacatgctgacaaggaagatggctccccattggaaagcgcagtgccgcctcctttccctggtccatgcacacttgggactggtttggcaagatacgcagaccttttgaaccctcctcgaaagtctgcgttagttgccttggcggcctatgccactgaaccaagtgaagccgagaaacttaagcacctgacatcacctgatggaaaggatgagtactcacaatggattgttgcaagtcagagaagtcttttagaggtgatggctgcttttccatctgcaaaacccccactaggtgtattttttgctgcaatagctcctcgtctacaacctcgttactactccatctcatcctcgccaagattggcgccaagtagagttcatgttacatccgcactagtatatggtccaactcctactggtagaatccacaagggtgtgtgttctacgtggatgaagaatgcagttcctgcggagaaaagtcatgaatgtagtggagccccaatctttattcgagcatctaatttcaagttaccatccaacccttcaactccaatcgttatggtgggacctgggactgggctggcaccttttagaggttttctgcaggaaaggatggcactaaaagaagatggagaagaactaggttcatctttgctcttctttgggtgtagaaatcgacagatggactttatatacgaggatgagctcaataattttgttgatcaaggcgtaatatctgagctcatcatggcattctcccgtgaaggagctcagaaggagtatgttcaacataagatgatggagaaggcagcacaagtttgggatctaataaaggaagaaggatatctctatgtatgcggtgatgctaagggcatggcgagggacgtccaccgaactctacacaccattgttcaggagcaggaaggtgtgagttcgtcagaggcagaggctatagttaagaaacttcaaaccgaaggaagatacctcagagatgtctggtga

SEQ ID NO: 51 - DNA, 2079 bp, synthetic construct
  source 1..2079: mol_type = other DNA
atgacgtcagctctgtatgcctcagacctgtttaaacagctgaagtcaatcatgggcacggacagcttaagtgacgatgttgtgcttgtgatcgctacgacctcattagccttagtcgctggctttgtagtcctgctgtggaagaaaacgacggcggaccgttctggtgagcttaaacccctaatgataccgaaatctcttatggccaaggacgaggatgatgatctggatttgggcagcggcaaaacacgtgttagtatattcttcggtacccagacggggacggctgaagggtttgcgaaagccctgagtgaagaaatcaaagccagatacgagaaagccgcggttaaagtgatcgatcttgacgattacgcggcggacgatgaccaatacgaagagaagctaaagaaagagacgctggcgtttttttgcgttgctacgtatggggacggcgagccgaccgataacgcagcacgtttttacaagtggtttacagaggagaatgagagagacataaagctgcaacaattagcctacggggtttttgctctgggaaataggcaatacgagcactttaacaagataggtatcgtgttggacgaagagctatgcaaaaaaggcgctaaacgtttaatcgaggtgggtttaggagacgatgatcaatccatagaagatgatttcaatgcatggaaggagtccttgtggtcagagttagacaaattactaaaggacgaggacgacaagagtgttgccactccgtacacagcggtcatacctgagtacagggtagtgactcacgatcccaggttcaccactcagaagtctatggagtccaatgtagccaacggcaataccacaatcgacatacaccatccttgcagggtagacgtggctgtgcagaaggaattacacacccatgaatctgaccgttcatgcatccacttagagttcgatattagccgtaccgggattacctatgaaacaggtgatcacgtaggcgtctatgccgaaaaccacgttgaaatcgttgaggaagcaggaaaattactgggtcacagtctggatttggttttttcaatacatgcggataaagaagacggttccccccttgagtctgcagttcccccaccttttccgggaccgtgtacgttaggtactggccttgcaagatatgcggatctattgaatccccccaggaaatctgcgctagtagctctggcagcctatgcgaccgaacccagcgaggcggaaaagttaaagcatctaacttcccctgacggcaaggacgagtacagtcaatggattgtggcgtcccaaagaagtctgctagaagtcatggcagcattcccttctgcgaaaccccctcttggggtgttttttgcagccatcgcccctcgtctacaaccgcgttactacagcatctcatcttctcccagattggcaccaagccgtgtccacgttacgtctgcattggtgtatggcccgactccaaccggtaggatccataagggtgtctgctctacctggatgaagaatgcggtgccagctgagaaatcacacgagtgcagcggcgccccgatttttatcagagcgtcaaacttcaaattgcccagtaatccgagtacccctatcgtaatggttggccctgggacaggtctagcaccctttagaggtttcctacaggagaggatggcattaaaggaagatggtgaagaacttggaagctctttactgttcttcgggtgccgtaataggcagatggatttcatatacgaggatgagttaaacaatttcgtagatcaaggggttatatccgaactaattatggcattctcaagagagggcgcgcagaaggagtacgtacaacataaaatgatggagaaggcggcgcaagtctgggacttaatcaaagaagaaggttatctttacgtctgcggagatgcaaaaggaatggcaagagatgtgcacaggactttgcacacaatagttcaagaacaagaaggcgtcagttcctccgaagccgaagcgatcgtgaaaaagttacagacagagggtaggtatttgcgtgatgtctggtga

SEQ ID NO: 52 - protein, 706 aa, Lotus japonicus
MEESSSMKISPLDLMSAMIKGTLDPSNVSSTSGAGSVFLENREFVMVLTTSIAVLIGCVVVFIWRRSTGNKAKSIEPPKRVVEKLSDEAEVDDGTRKVTIFFGTQTGTAEGFAKAIAEEAKVRYEKAKFKIVDMDDYAQDDDEYEEKLKKETLALFFLATYGDGEPTDNAARFYKWFLEGDEKEEGWLRNLEYAVFGLGNRQYEHFNKVAIEVDDKLADFGGKRLVKVGLGDDDQCIEDDFTAWKEELWPALDELLRGDDDTTVSTPYTAAVLEYRVVIHDPLDASVDEKKWHNVNGHAIVDAQHPVRSNVAVRKELHTPVSDRSCTHLEFDISGTGVAYETGDHVGVYCENLSETVEEAVRLLGLSPDTYFSVHTDDEDGKPLSGSSLPPTFPPCTLRTAIARYADVLSSPKKSVLLALAAHASNPSEADRLRHLASPAGKDEYSEWVIASQRSLLEVMAEFPSAKPPIGVFFAAIAPRLQPRFYSISSSPRMAPSRIHVTCALVNDKMPTGRIHRGVCSTWMKNSVPLEKSQDCSWAPIFVRQSNFKLPADNKVPIIMIGPGTGLAPFRGFLQERLALKEDGAELGPSVLFFGCRNRQMDYIYEDELNHFVNSGALSELIVAFSREGPTKEYVQHKMMEKASDIWNMISQGAYIYVCGDAKGMARDVHRTLHTILQEQGSLDSSKAEGMVKNLQLNGRYLRDVW

SEQ ID NO: 53 - DNA, 2121 bp, Lotus japonicus
  source 1..2121: mol_type = genomic DNA
atggaagaatcaagctccatgaagatttcgcctctggatctgatgtccgccatgatcaagggcacactcgacccttccaacgtctcctccacctccggcgccggctccgtcttcctcgagaatcgtgagttcgtcatggtgcttaccacctccatcgccgtcctcatcggatgcgtcgtcgttttcatttggcgcagatccaccggtaacaaggctaagtccatcgagcctcccaagcgcgtcgtcgagaagcttagcgacgaggctgaggttgacgacggtaccagaaaggtcaccatcttcttcggtactcagactggtactgctgaaggattcgccaaggcgattgcggaagaggcaaaagtgcgatacgaaaaagccaagttcaaaattgttgatatggatgattatgcccaggacgatgatgagtatgaggaaaagctcaagaaagagacactggcacttttcttcttagctacatatggtgatggtgagccaactgataatgcggcgagattttacaaatggtttctggagggagatgagaaagaagaaggatggcttcgaaatcttgagtatgctgtttttggtctggggaacaggcagtatgagcattttaataaggtcgccattgaagtggatgataagcttgctgattttggtgggaagcgtcttgtcaaagtaggtctaggagatgatgatcaatgcatagaagatgactttactgcatggaaagaagaattgtggccagcattggatgaattgcttagaggtgatgatgatacaactgtgtctacaccctatacggctgctgtgttggagtatcgtgttgttattcatgatccattagatgcatctgtcgatgaaaagaagtggcataatgttaatggccatgctattgtggatgctcaacatccagtcaggtcaaatgtggctgtgcgaaaggagcttcatactcctgtgtcagatcgttcttgcacacatttagaatttgacatttcaggcactggagttgcatatgaaacaggggaccatgttggtgtttactgtgagaatttatctgaaactgtggaagaagcagtaaggttactaggtttgtcaccagatacctatttctccgtccatactgatgatgaagatgggaaaccacttagtggaagctccttgccacctactttcccaccatgtactttaagaacagcaattgcccgatatgcagatgtcttgagttcacccaaaaagtctgttttgcttgccttagctgctcatgcatctaatccatctgaagccgaccgcctacgacatcttgcttcacctgctggaaaggatgaatattcagagtgggtgattgccagtcaaagaagtctccttgaggtcatggctgaatttccatcagccaaacctccaattggtgtctttttcgcagcaattgctcctcgcctgcagccaagattttattcgatctcatcatctcctaggatggctccatccagaattcacgttacctgtgcattagtgaatgataaaatgcccactggtaggattcataggggagtgtgttcaacatggatgaagaattctgtgccattggagaaaagtcaggactgcagttgggctccaatatttgttagacagtccaattttaaactccctgctgataataaagtgcctataatcatgataggtcctggcacaggattggctcctttcaggggtttcttgcaggaaagattagctctgaaagaggatggagctgaacttggcccctccgttttattcttcggatgcaggaatcgtcaaatggactacatctatgaagatgagttgaaccactttgtcaacagtggtgcgctttctgagctcattgttgccttctcacgggagggacctaccaaggaatatgtgcaacataaaatgatggagaaggcttcggatatttggaacatgatatctcagggagcttacatttatgtgtgtggggatgccaagggcatggctagggatgtgcaccgtactctgcacacaattttgcaagagcagggttctctcgatagttccaaggctgagggtatggttaagaacctacaattgaatggtaggtatttgcgtgatgtatggtga

SEQ ID NO: 54 - DNA, 2121 bp, synthetic construct
  source 1..2121: mol_type = other DNA
atggaagaaagttcttctatgaagatctcacctcttgacttgatgtcagcaatgatcaaggggactcttgacccttccaatgtctcatctacctcaggcgcggggtcagtcttcctggagaatagggaatttgtcatggtgttaacgacatccatagcggtcctgatcggatgtgtcgtagtattcatatggaggagaagcacgggaaacaaggccaaatctatcgaaccccctaaaagagtagttgagaagctatcagatgaagcggaggtagatgatggtactaggaaagtaactattttttttgggactcagactggtacggcggaaggatttgcgaaagctatagctgaagaagcaaaggtacgttacgagaaggctaaattcaaaattgtggatatggatgactatgcacaagacgatgatgagtatgaggagaaattgaaaaaggaaacccttgcgttattttttttagcaacttacggggacggagaacctacggacaacgccgcaaggttttacaaatggtttctagaaggagatgagaaagaagaaggctggctacgtaacctagagtatgcagtatttggattgggtaataggcagtatgagcatttcaataaagtagccatagaagttgacgacaagctggccgatttcggtggcaagagattggtcaaggtgggtctgggtgatgatgaccagtgtatcgaagatgattttactgcgtggaaagaggagttgtggcccgcactggacgaactgttgaggggggatgacgataccactgtgtccacaccatatactgccgctgtcttagaataccgtgtagtcatccatgaccctctagatgcatcagtcgatgaaaagaagtggcacaatgtcaacggtcatgccatcgtggatgcgcagcatccggtaaggagtaacgtcgctgttagaaaagagctgcacacacctgtgagcgataggagttgtacccaccttgaattcgacataagcggaacaggagtagcttacgagacgggagatcatgtgggcgtatattgcgagaacctttcagaaacggttgaggaagcggtccgtctgcttggtctttccccagacacctatttttccgtacacactgacgatgaagacgggaagcctttgtccggaagctcactgcctccaacgtttccaccatgcacgcttcgtaccgcaatagcgcgttatgctgatgttcttagctcaccaaaaaaatctgtactacttgctctggcggctcatgcctcaaatccgtcagaggccgatagattgaggcatttagcttccccggcaggaaaggatgaatattctgaatgggttatagcatcacaaagaagtttgttagaagtcatggcagagtttccaagtgcaaagccaccaatcggcgtgttcttcgccgctatcgcacctagactgcagccaagattttactccatcagttcatctccgagaatggcgccctccaggatacacgtcacatgcgcactggtaaatgacaagatgcctacggggagaattcataggggtgtgtgcagtacgtggatgaagaacagtgtgccactggagaaaagccaggactgtagctgggcacccatatttgtccgtcagtctaatttcaagctaccagcagacaacaaagtcccaattataatgatcggccccggcacggggttggctcctttcaggggcttcttgcaagagaggctagctcttaaggaggatggcgcggaactgggtccctccgtcctgttttttgggtgcaggaacagacagatggactacatttacgaggacgaattaaatcacttcgttaactccggggcactgagcgagttgatcgtcgcgtttagtagagaaggacctactaaggaatatgtgcagcataagatgatggagaaggcgtctgacatctggaacatgatctcacagggggcatacatttatgtatgtggtgatgctaaggggatggctagagacgttcacaggaccttacacaccatactgcaagaacaaggtagtcttgactcttccaaggcggaaggcatggtaaagaatctgcagcttaatgggaggtaccttagggatgtttggtga

SEQ ID NO: 55 - protein, 278 aa, Quillaja saponaria
MDFLQSHRAEMLSMGMALVAIVGSTAYYFYVTKKPKGSLDPNNFKEFKLVKRTQLSHNVAKFRFDLPTSTSVLGLPIGQHMSCRGKDNLGEEVIKPYTPTTLDSDVGYFELVIKMYPQGRMSHHFREMREGDHLAVKGPKGRFKYQLNQVRAFGMLAGGTGITPMFQVTRAIVENPSDKTNIHLIYANVTYEDILLKEELDALAINFPSRFRIYYVLNQPPGGWEGGVGFVTKEMIQTHCPAPAPDIQVLRCGPPPMNKAMAVHLDDLGYSRQMQFQF

SEQ ID NO: 56 - DNA, 837 bp, Quillaja saponaria
  source 1..837: mol_type = genomic DNA
atggattttttgcagtctcacagggcggagatgctaagcatgggaatggctcttgtagccattgttggtagtactgcttactatttctatgtcaccaagaaacctaaaggtagcttggaccccgataacttcaaggaatttaaacttgttaagcgtacacaactcagtcataacgtggctaagtttagatttgaccttcctacatcaacctctgtgctgggacttcctattggacaacatatgagctgcagagggaaagataatcttggtgaagaagttatcaaaccttatactccaacaactttggattctgatgttggatactttgaattagttataaagatgtatccacagggtaggatgtcccaccattttcgagagatgcgcgaaggtgatcacctagctgtgaaaggacccaagggacgcttcaagtatcagctcaaccaagtcagagcttttggaatgcttgctggtggcactgggatcactccaatgtttcaggttacaagagccatagtagaaaatccaagtgacaaaaccaacatacatcttatatatgccaatgtcacatatgaggacattcttttgaaggaagagcttgatgctcttgccattaactttcctagtcgattcagaatctactatgttctaaatcagccacctggaggatgggagggtggtgtcggttttgtaacgaaggaaatgattcaaacacactgccctgcaccagcaccagatattcaggtattaagatgtgggccacctcccatgaacaaagctatggctgttcatcttgatgatcttggatacagccgccaaatgcagtttcagttctaa

SEQ ID NO: 57 - DNA, 837 bp, synthetic construct
  source 1..837: mol_type = other DNA
atggatttcctacagtcacacagggctgaaatgttaagcatgggaatggcgttggtggcgattgtggggtctactgcgtactacttttacgtgacgaaaaagcccaagggttccttagatccaaacaatttcaaagagttcaaattagtgaaacgtactcaattatctcacaacgtcgccaaatttagatttgatcttccgacttcaacgagcgtccttggccttcccatcggtcagcatatgtcatgtaggggtaaagacaacctaggcgaagaagttataaaaccgtacacccctacaacgctggattccgacgttggttacttcgagttagtaattaaaatgtatccgcagggaaggatgtctcatcacttccgtgaaatgagggagggggatcacttagctgttaaagggcccaaagggagatttaagtatcagttaaaccaggtgagagcgtttgggatgctggcaggcggcacagggatcactccaatgtttcaggtaactcgtgcgattgttgaaaatccgtctgacaaaacaaatatacatctaatatatgcgaacgtgacatacgaggatattttgttgaaagaagaattggacgcccttgctatcaatttcccatcaaggttccgtatttattacgtcttaaatcaaccccctgggggctgggaaggaggggtgggttttgtaactaaggaaatgatccagacacactgccctgcgcccgcacctgacatacaagtccttcgttgcggccctccgccgatgaacaaggccatggcggtgcacttggacgatcttggctacagtcgtcagatgcaatttcaattttaa

SEQ ID NO: 58 - protein, 281 aa, Arabidopsis thaliana
MDTEFLRTLDRQILLGVFVAFVAVGAGAAYFLTSSKKRRVCLDPENFKEFKLVKRHQLSHNVAKFVFELPTSTSVLGLPIGQHISCRGKDGQGEDVIKPYTPTTLDSDVGRFELVIKMYPQGRMSHHFREMRVGDHLAVKGPKGRFKYQPGQFRAFGMLAGGSGITPMFQVARAILENPTDKTKVHLIYANVTYDDILLKEELEGLTTNYPEQFKIFYVLNQPPEVWDGGVGFVSKEMIQTHCPAPASDIQILRCGPPPMNKAMAANLEALGYSPEMQFQF

SEQ ID NO: 59 - DNA, 1521 bp, Arabidopsis thaliana
  source 1..1521: mol_type = genomic DNA
cacaaattgtaattgaaaaaacgaagatatttttttctttagaaaaattatactaagaaaaaaaaattggtcaaaaatgtaacaaatatgttaatggaataataatgtgttgcaaaagaggcaaactatacaacgtgaaagtggtaggtctaccagatcccataccctcattttaatggcggagattacaagggaaagagacaactccaattcaaagctctgattttttccaccaatccccattttttcccttttacaattcttaagctagttttatacttttcttcttcctttcatttgggttaagagaagccatggataccgagtttctccgaaccctagatcgtcagattcttttgggtgtcttcgttgctttcgtcgccgttggtgctggtgctgcttattttcttacatcctccaagaaacgcagagtgtgtttggatccagagaatttcaaggagttcaagcttgttaagagacatcagcttagtcacaatgtggccaagttcgtttttgaactcccaacttctacttctgtgttgggtcttcccattggacaacacatcagttgcaggggaaaggatggtcaaggagaggatgttattaagccatacaccccgactacgttagactctgacgttggacgtttcgaacttgtcattaagatgtatccgcaaggacggatgtctcatcatttcagggagatgcgtgttggagaccatcttgccgtaaagggaccaaagggtaggttcaagtatcaaccaggtcagtttagggcatttggaatgcttgctggaggttcaggcatcactcccatgttccaagtggccagagcaattctagaaaacccaacagacaagacaaaggtgcatctcatttacgccaacgtcacatacgacgacattctcttgaaggaagaattggagggtcttactaccaattaccctgaacaatttaaaatcttctatgttttgaaccagcctccggaagtatgggatggtggtgttggatttgtatcaaaggaaatgattcagactcattgccctgcacctgcatctgatattcagatcctaagatgcggaccaccgccaatgaacaaggccatggctgcaaaccttgaagctctgggatactctccggagatgcaattccagttctgatctgaatatcttcttttggttgctctgaaacggaactcataataatttagttacaatcccaactacgagagttgtgtcttaactgagtgagagttgtatttctgttgtgtgtttgttagcccacttgaaagtttccattgattggtttacgaattggttagacattttggttcacctataccggtttggtgcaaacactttgatgcttgatagtgagtggcgacatactgacatgacctaccctatgtgttttggtatgttgctatcaatttttggctactagtctatacgtgaatcatcatcaatccactttttaaaagcaaaactgaaaaggatgattgaaaatataatattattggta

SEQ ID NO: 60 - DNA, 846 bp, synthetic construct
  source 1..846: mol_type = other DNA
atggatactgagttcttgagaactcttgacaggcaaattttgctaggagtcttcgtggcgttcgtcgcggttggtgcgggggctgcgtattttctaacttccagcaaaaaacgtagagtctgccttgatcctgagaactttaaagaattcaaattggtcaaaaggcaccaattgagtcacaatgttgctaagtttgtgtttgagttacctacgtccacgtccgtcttaggtttacccattgggcagcacattagttgtagagggaaagatgggcaaggtgaagatgtcataaagccatacacccctacgacgcttgactcagacgtgggtaggtttgaactagttattaaaatgtacccacaaggtcgtatgagtcaccatttcagagagatgagggttggggaccatttggcggtaaaggggcctaaagggcgtttcaagtaccaaccaggacagttcagagcatttggcatgttagctggaggctccgggataactccgatgttccaggtggctagagctattctggaaaacccgacggacaagaccaaagtccaccttatttatgcaaacgtcacctacgatgatatcttgttaaaggaggaacttgagggtttgaccaccaactatcctgagcagtttaaaatattttatgtcctgaatcagccacccgaggtatgggacgggggggttgggttcgtttcaaaagaaatgattcaaactcattgtccagcgccggcatccgatatccaaatacttaggtgcggcccaccacccatgaacaaagccatggcagcaaatctggaggctctaggttattctccggaaatgcaattccagttttaa

SEQ ID NO: 61 - protein, 276 aa, Saponaria vaccaria
MEVLQTTEMQILAIGVAVVVVIGTAAYFFSSKNKGSLDAEKFKEFKLVKKEQLSHNVAKFTFGLPTPTSVLGLPIGQHISCRGKDSQGEEVMKAYTPTTLDCDLGRFELVIKMYPQGRMSHHFREMKVGDYLSVKGPKGRFKYQPGQVRAFGMLAGGSGITPMYQVSRAILENPSDKTKVQLIYANVTYEDILLKEELDNLAKTYPDRFSVYYVLNQPPEGWKGGVGFVSKEMIKENCPAPAADIQILRCGPPPMNKAMAAHLEELGYAPEMQFQF

SEQ ID NO: 62 - DNA, 831 bp, Saponaria vaccaria
  source 1..831: mol_type = genomic DNA
atggaggtgctacaaacaacagagatgcaaatactagcaattggtgttgcagttgttgtagtcattggcaccgctgcttacttcttttcctctaaaaacaaaggaagtttggatgcggagaaattcaaggaatttaagctggtgaagaaagagcaattgagtcataatgtggctaagttcacatttgggctcccgactccaacttctgttttgggtcttcctattggtcaacacatcagttgcaggggtaaagatagccaaggtgaagaagtcatgaaagcatacacaccaactacattggactgtgatcttggacgctttgagctggttatcaagatgtatcctcaaggaagaatgtcacaccactttcgagagatgaaggttggggattatctatctgttaagggaccaaagggacgtttcaagtaccagcctggtcaagttagagcattcgggatgcttgctggaggttccggaatcactcctatgtatcaagtttctagagccatattggagaatccaagcgacaaaactaaagtgcaacttatctacgctaatgtaacatatgaagacattctgttaaaggaagaactggacaatcttgcaaaaacttatcctgatcgcttcagtgtttattacgtccttaaccaacctccagaaggatggaaaggtggtgttggatttgtaagcaaggagatgatcaaagaaaactgtcctgcacctgcagcggatattcagatcctaaggtgtggcccacctccaatgaacaaagctatggctgctcacctcgaggagctcggttacgccccggaaatgcagtttcagttctga

SEQ ID NO: 63 - protein, 220 aa, Arabidopsis thaliana
MALELWQTLKEAIHAYTGLSPVVFFTALALAFAIYQVISGWFASPFDDVNRHQRARSLAQEEEPPIPQPVQVGEITEEELKQYDGSDPQKPLLMAIKHQIYDVTQSRMFYGPGGPYALFAGKDASRALAKMSFEEKDLTWDVSGLGPFELDALQDWEYKFMSKYAKVGTVKVAGSEPETASVSEPTENVEQDAHVTTTPGKTVVDKSDDAPAETVLKKEE

SEQ ID NO: 64 - DNA, 663 bp, Arabidopsis thaliana
  source 1..663: mol_type = genomic DNA
atggcgttagaactatggcaaactctcaaagaagcaatccatgcttacacaggtctttctcctgttgtcttcttcactgctctagctctcgccttcgccatttaccaagtcatctcaggctggtttgcctcgccgttcgatgatgttaaccgacatcagagagctagatccttggctcaagaggaggagccaccgattcctcagcctgttcaagtcggtgagatcacggaggaggagcttaaacagtacgatggctctgatcctcaaaagccccttcttatggctatcaaacatcagatctatgatgttacacaaagcaggatgttctacggaccaggaggaccatatgctttgtttgcagggaaagacgctagccgagctcttgcaaagatgtcatttgaggagaaagacttgacttgggatgtctctggtcttggtccctttgagctagatgctcttcaagattgggagtacaagttcatgagcaagtatgctaaggttggtactgtcaaagtggctggttcagaacctgaaaccgcatctgtctctgaacccacagagaatgttgagcaagatgctcatgtaaccacaacgcctgggaagaccgttgttgataagagtgatgatgctcctgctgagactgtgttgaagaaggaggagtag

SEQ ID NO: 65 - protein, 233 aa, Arabidopsis thaliana
MVQQIWETLKETITAYTGLSPAAFFTVLALAFAVYQVVSGFFVSPEVHRPRSLEVQPQSEPLPPPVQLGEITEEELKLYDGSDSKKPLLMAIKGQIYDVSQSRMFYGPGGPYALFAGKDASRALAKMSFEDQDLTGDISGLGAFELEALQDWEYKFMSKYVKVGTIQKKDGEGKESSEPSEAKTASAEGLSTNTGEEASAITHDETSRSTGEKIAETTEKKDVATDDDDAAKE

SEQ ID NO: 66 - DNA, 702 bp, Arabidopsis thaliana
  source 1..702: mol_type = genomic DNA
atggttcagcaaatatgggagacgttgaaggaaacaatcacagcttacactggactttctccagctgcgtttttcaccgtacttgctctcgctttcgccgtttaccaagtcgtctccggtttcttcgtttctcctgaagttcaccgacctcgttctttggaggttcagcctcaatcggagcctcttccaccgccggttcagctcggagaaatcactgaggaggagcttaagctttatgatggctccgattctaaaaagccccttcttatggcgatcaagggccagatctatgatgtttctcagagcaggatgttttatggaccaggtgggccatatgctctgtttgcagggaaagatgcaagccgagctctggcaaagatgtcatttgaggaccaagacttgactggagacatctcaggtctcggtgcatttgagctagaggcgttacaagactgggagtacaagttcatgagcaagtatgtcaaagtcggaaccattcaaaagaaggatggagaaggcaaagaaagttcagaaccttccgaagcaaagactgcctctgcggaaggtctttctacaaacactggagaagaagcttcagcaattacccatgatgaaacttctagaagcacaggcgagaaaatcgcggaaaccacggagaagaaagatgttgcaactgatgatgatgatgctgcaaaggagtaa

SEQ ID NO: 67 - protein, 225 aa, Saponaria vaccaria
MSIEIWETLKEAITTYTGLSPTLFFTIVALSLAFYHAVFGLFGSSSSSSSSSSSNSHQNPRNFVEESEPLPPPVQLGEITEDDLKNYDGSDSKKPLLMAIKGQIYDVSQSRMFYGPGGPYALFAGKDASRALAKMSFEDKDLTGDISGLGPFELEALQDWEYKFMSKYVKVGTIKKDAPPSDAPSPSEPSEAADVTDREAPKHAEDGPAETVEPSSVGDAEKKEE

SEQ ID NO: 68 - DNA, 678 bp, Saponaria vaccaria
  source 1..678: mol_type = genomic DNA
atgtcaatagaaatatgggaaacactgaaagaagcaataacaacatacacaggactatcaccaacactattcttcacaatagttgcacttagtcttgcattctaccatgctgtttttgggttatttggttcatcatcatcatcatcatcatcatcatcatcaaatagccaccaaaatccaaggaattttgttgaggaaagtgagcctttaccaccacctgtgcaacttggtgagattactgaggatgacttgaaaaactatgatggctctgattctaagaagcctttgcttatggccattaaaggccagatctatgatgtttcccaaagcaggatgttttatggtccaggtgggccatatgcattgtttgcaggaaaagatgcaagcagagctctggcgaagatgtcgtttgaggataaagatctgacaggcgatatctctggtctcggtccattcgaactcgaggccttacaggactgggagtacaagttcatgagtaaatacgtcaaggtcggaactatcaaaaaggatgctcctcccagtgacgcaccgtctcctagtgaaccctctgaagctgctgatgttacagacagggaagctcccaaacacgcagaagatggcccagccgagacagtagaaccatcctctgttggtgatgccgagaaaaaagaagagtaa

SEQ ID NO: 69 - DNA, 678 bp, synthetic construct
  source 1..678: mol_type = other DNA
atgtccattgagatttgggagacgttgaaggaagccatcacgacttacacaggattgagccccacactgttcttcactatcgtggctttgagtttagctttctaccatgccgtcttcggtttgtttgggagcagtagttctagctcttcatccagttcttccaatagtcaccagaacccacgtaacttcgtggaggagtcagagccactgccaccgcccgtgcagctaggcgagatcactgaggacgacctgaagaattatgacggcagcgactctaagaagcctctactaatggcgatcaaaggtcagatttatgacgtgtctcagagcagaatgttctacggtccaggtgggccttacgccctttttgcaggaaaggatgcttccagagctcttgctaaaatgagtttcgaagacaaagatttgacgggggacatttccggtctaggacctttcgagttggaagccctgcaagactgggaatacaagttcatgtccaagtacgtaaaagttggcactattaagaaagacgcccctccatccgacgccccaagcccttctgaaccctcagaggcagcagatgtaacagacagggaagcacccaagcacgctgaagacggacctgctgagacggtcgaaccgagctccgttggagacgctgaaaaaaaggaagagtga

SEQ ID NO: 70 - protein, 189 aa, Saponaria vaccaria
MQLVGLRNIYGMLVEAIHDYFGLSPTAFLVIVGIMIVTYKIVCGMFVAVDDYKAVRDMKAVFEEDMRREPVQLGDVTEEELKAYDGSDPKKPLLMAIKGQIFDVSRGRMFYGPGGPYAMFAGHDATRALALMSFDPQDLTGNTDGLSESEIDVLEDWELKFNEKYPKVGVLVSKAAIVDNQPLDTKKDA

SEQ ID NO: 71 - DNA, 570 bp, Saponaria vaccaria
  source 1..570: mol_type = genomic DNA
atgcaattagtcgggctaagaaatatatacgggatgttggtggaagccatacatgattatttcggtctgtctccaacggcattcttggtcattgtgggcataatgatagtaacttacaagattgtgtgtggaatgtttgtggccgtcgatgattacaaggcagtgagagatatgaaagcggtgtttgaggaggatatgaggcgggagccagtgcagctgggcgacgtaacggaggaggaactcaaggcttatgacgggtctgacccaaagaagccgttgctgatggcgatcaagggacagattttcgacgtgtcccggggcagaatgttctacggtcctggtgggccctatgccatgtttgctggtcatgatgccaccagagcgctagctcttatgtcttttgatccacaagatctaaccggaaacaccgatggcctcagtgaatctgaaatagacgtcctcgaggattgggagctcaagttcaatgagaaatatcccaaggttggcgtacttgtatccaaagctgctatcgtcgacaatcagcctcttgacactaaaaaagatgcttag

SEQ ID NO: 72 - DNA, 570 bp, synthetic construct
  source 1..570: mol_type = other DNA
atgcaactggtgggcttgagaaatatctacggcatgctggtcgaagcaatacacgactacttcggtctttctcctaccgcatttctagtgatagtgggcattatgatcgttacatataagatcgtgtgtgggatgtttgtggcggtggacgactataaggcggtgcgtgacatgaaggcggtgttcgaggaagatatgagacgtgaaccggtccaattgggggatgtgaccgaggaggagctgaaagcatacgatggtagcgaccctaaaaagccacttcttatggcgatcaaagggcagatttttgacgtttccagaggaagaatgttttatgggcctggcgggccttacgcaatgttcgcgggccatgacgcgacaagggcacttgcactgatgagctttgacccgcaggatctgacggggaacactgatggtctgagcgaaagtgagattgacgtacttgaggactgggaattaaaattcaacgaaaaatatccgaaggtcggggtgcttgtttcaaaggctgctatagttgacaaccaacctcttgatactaaaaaggatgcataa

SEQ ID NO: 73 - protein, 200 aa, Quillaja saponaria
MGVYLTVMEEITWYTGLSPAACFTILAMMVVVYRTVCGFFVAPEDYNKPPIITTRNNSSFDKVNPSTPEPVQLGEISEQELRAYNGSDPNKPLLMAIKGQIYDVSSSRMFYGPGGPYAMFAGKEASRALALLSFKPEDINGNLEDLEEPELEILQDWEDKFIEKYVKVGQLVPEQKTQQTEHGEKLQEHDSQNQESVEAQ

SEQ ID NO: 74 - DNA, 603 bp, Quillaja saponaria
  source 1..603: mol_type = genomic DNA
atgggtgtgtacttgacggtaatggaggagataacatggtatactgggctctctccggcagcctgcttcaccatcctcgctatgatggtggtggtttatagaactgtatgcggtttttttgtggctccagaagattacaataagcctccaatcattacaacccgaaacaattctagcttcgacaaagtcaatcccagcacacctgaaccggttcaattgggagaaattagtgagcaggaattgcgagcctataacggttcagaccccaataagcctcttttaatggcaatcaagggtcagatctacgacgtctctagctctaggatgttttatggtcctggtggaccgtatgcaatgtttgctggaaaggaagctagcagagccctagctcttctatcttttaaacctgaagacattaatgggaaccttgaagatttggaagaaccagagcttgaaattttacaggattgggaggataagtttatagaaaagtatgtgaaagttggtcagcttgttccagaacaaaagactcagcagactgagcacggagaaaaactccaagaacatgatagccagaatcaggaaagtgttgaagcacaataa

SEQ ID NO: 75 - DNA, 603 bp, synthetic construct
  source 1..603: mol_type = other DNA
atgggcgtttatttgacggtcatggaagaaatcacatggtacacaggattaagtccagccgcgtgctttacaatattggctatgatggtggtcgtctacagaacagtttgcggtttctttgtagcgccagaggactataataaaccgccaataatcacgaccaggaacaattccagttttgacaaggtaaatccatccacccctgagccagtgcaattgggggaaattagcgaacaggaacttagggcatataatggctccgatcccaacaagccacttctaatggccattaaaggccaaatatatgacgtatcatcaagtcgtatgttctacgggccgggaggaccatacgccatgtttgcgggcaaggaggcaagtagggccctagcccttcttagtttcaagcccgaggacatcaatgggaaccttgaagacctggaagaaccagaattggagatattacaagactgggaggataagttcattgaaaagtacgtgaaagttgggcagctagtgccggagcagaaaactcaacaaacagaacatggcgaaaagttgcaagaacatgactcacagaatcaagaaagcgtagaggcacagtaa

SEQ ID NO: 76 - protein, 703 aa, Saponaria vaccaria
MSPYNNTCTLQTTRAILSRIHILLHSSLIASALYYRVTTFVTNPSVGLPWVLMALAELTLAFVWALTQAFRWRPVVRAVAGAEDIDDTRLLGLDVFICTADPRKEPVVEVMNSVISALALDYPADKLAVYLSDDGGSPLTREVIREAAEFAKLWVGFCGKYRVKTRCPEAYFSAFCDAERVDHNVDYKNDELVIKSKFEAFKKYVEKASEDASKCIVVNDRAPRVEIIHDNKENGESELKMPLLVYVAREKRPHFPHHAKAGAINTLLRVSGLLSNNPIFLVLDCDMYCNDPTSARQAMCFHLDPKLAPSLAYVQYPQIFYNTSKNDIYDGQARAAFKTKYQGMDGLRGPVMSGTGYFLKRKALYGKPHNEDELLKEQPTKAFGSSRTFIASLSEDSHGALKELSKEKLLQEAENLATCTYESNTLWGSEVGYSYDCLLESTYCGYLLHCKGWISVYLYPKKPCFLGCATVDMNDAMLQIMKWTSGLIGVGISKFSPFTYAMSRISIMQSLCYAYFAFSGLFTVFFLTYGLVLPYCLVQGIPLFPKAGDPWLLAFAGVFVSSLLQHLYEVLSSGEPVKTWWNEQRIWIIKSVTACLFGLLDAVLNKVGVLKASFRLTNKAVDKEKLNKYEKGRFDFQGARMFMVPLLILVVFNLVSFFCGLRRTVVHKNYDEMFAQLLLSLYSLALSYPIMEEIVRKARKGRA

SEQ ID NO: 77 - DNA, 2112 bp, Saponaria vaccaria
  source 1..2112: mol_type = genomic DNA
atgtcaccttataataacacctgcacactccaaactacccgagccatactcagccgcatccacattctcctccactcctccctcatcgcctcggctctctactaccgcgtcaccaccttcgtcaccaacccgtctgtaggcctcccatgggtcctcatggccttggcagagctcacccttgcgttcgtatgggccctcacgcaggcgttccgttggaggcctgttgtgagggctgtcgccggggctgaggacattgacgacactcgtctcctgggtttggacgtgtttatttgcactgctgacccgaggaaggagccggtcgtcgaggtcatgaactctgtgatatcggctttggcgttggattatcctgctgataagctggcggtctacttgtcggatgacggggggtccccgttgactagggaggttattagggaggcggccgagtttgccaagttgtgggtcgggttttgtgggaagtatagggtgaagactaggtgccctgaggcgtacttctctgcgttctgtgatgctgagagggttgatcataatgtggactataagaatgatgaacttgtcatcaagtcgaagtttgaagcatttaagaaatatgtagaaaaggcaagcgaagacgcaagcaaatgtattgtagtcaatgaccgtgctcctcgcgttgagattattcatgacaacaaggagaatggagaaagtgaactgaaaatgcctctgcttgtttatgtagcaagggaaaaacgaccacattttcctcaccatgccaaagcaggagccattaatacactccttcgagtgtcgggtctactgagcaataaccctatctttttggtgctggattgtgatatgtactgtaatgatccaacctctgcccgtcaagctatgtgcttccatcttgacccgaagcttgctccctctctagcttatgtgcaataccctcaaattttctacaacacgagcaaaaatgacatctatgacggtcaggccagagcagcatttaagactaaatatcaaggcatggatggtcttagagggccggtcatgagtggcacggggtattttttgaagaggaaagccttgtacggaaaacctcataacgaagatgagctacttaaggagcagccgacgaaggcgtttggctcctctagaacattcatcgcatcccttagtgaagatagccatggtgccttgaaagaattgagtaaagagaagttgctgcaagaggctgaaaatttggctacttgcacatacgaatcaaacacgttatggggtagtgaggttggatactcatatgactgcttgttggagagcacatactgtgggtatttgttacactgcaaaggatggatttcggtatatctatacccgaaaaagccgtgtttcttggggtgtgcaacagtcgacatgaatgacgccatgcttcagattatgaaatggacttccgggttgattggcgttggcatatcgaagttcagcccattcacttatgccatgtctaggatctccattatgcaatctctttgctatgcttacttcgctttttcgggcctatttaccgtcttcttcctcacgtacggccttgttcttccgtattgcctcgtgcagggcattccgctatttcccaaggcaggggatccatggcttctagcatttgcgggagtattcgtatcctcccttcttcagcacttgtatgaggtcctctcaagcggagaaccagtgaaaacatggtggaacgagcagagaatctggatcataaaatcagtaaccgcctgtctattcggtcttctggacgctgttcttaacaaggttggcgtcttaaaggctagtttcaggctgactaacaaggctgtcgacaaagaaaagctcaacaaatacgagaagggcagatttgatttccaaggcgcgagaatgtttatggtccctctcttgattttggtggtgtttaacttggtttcgttcttttgcggcctaaggaggaccgtggtccataagaattacgatgaaatgttcgctcagcttttgctctcgttgtacagtctagctcttagctacccaatcatggaggagattgtccgaaaagctagaaaaggtcgcgcctaa

SEQ ID NO: 78 - protein, 713 aa, Quillaja saponaria
MKSPSNPNQKPILHTCTIQQPRATLNKIHSLIHFSAILVLFYYRITRLFFTDDFKVPKLLWTLMTISEFILAFIWVLIQPFRWRPVSRSVIPENMPKDISLPAVDVFVCTADPQKEPTVEVMNTILSAMALDYPAEKLAVYLSDDGGSAVTLYAIKEACCFAKMWLPFCNKYGIKSRCPEAYFSKLAADEWLHRSVEFVAEEKEVKANYEEFKRNVQKFGEQQENSRVVHDRHPHVEIIHNNWNNEDQAHEMPLLVYVSRERRPSHHPRFKAGALNTLLRVSGIISNSPYILVLDCDMYCNDPTSARQAMCFHLDPQLSKNLAFVQFPQIFYNASKNDVYDAQVRAAYQTKWQGMDGLQGPIFSGTGFYLKRKAMYGNPDQDDNCLLKPYKKFGMSGEFVESLKVLNEQDGTQKKLLDGFLQEAKLLASCAYETKTSWGKEIGFSYDCLIESTFTGYLLHCRGWISVYLYPKRPCFLGCCPTDMKDAMVQYTKWMSELFSIAISRFNPLLYGVARMSILQSLCYGSFTLAPILSFPLFLYGTVPQLCLLKGISLFPKVSDPWFAVFAAIFVSSLCQHWFEVLSCDGTFTTWCNEQRSWLIKSVSGSLFGVVGAILQRLGLKTKFSLSNKAMDKEKLEKYEKGKFNFQGAAMFMVPVSILVILNTFCFLGGFWKVIIMKNILDMFGQLSLSAYVLVLSCPVLEGMLTRISKKMV

SEQ ID NO: 79 - DNA, 2142 bp, Quillaja saponaria
  source 1..2142: mol_type = genomic DNA
atgaaatccccctctaacccaaatcagaaacccatcctccacacttgtacaattcagcagcctcgtgctacccttaacaaaattcatagtcttattcatttctcagccatacttgtcctattttattaccggataacccgtctattcttcaccgacgatttcaaggtacccaagttactatggactctaatgacaatctccgagttcattcttgccttcatttgggttctcatccaacctttccggtggcgaccggtgtcccgttccgtcataccagagaatatgccgaaggacatcagtttgccggcggtggacgtgtttgtatgcacagctgaccctcaaaaagaacccacagtggaggtgatgaacacaattttatcagccatggctttagactacccggcggagaagctcgccgtgtatctttccgatgatgggggttctgctgtcaccttatatgctataaaagaagcttgttgttttgctaagatgtggcttccgttttgtaacaagtatgggatcaaatcaaggtgtcccgaggcttatttttcaaagcttgccgctgacgagtggcttcaccggagtgtggaattcgtggcagaagaaaaggaggtcaaggctaattatgaagagttcaagagaaatgtgcagaaatttggtgagcaacaagaaaacagtcgtgttgtgcatgatcgtcaccctcatgttgagattatacacaataattggaataacgaagaccaagctcatgagatgccactccttgtttatgtctctcgtgaaagaagaccatctcaccatcctcgattcaaagctggagctcttaacacccttcttcgagtttctggcatcatcagcaacagcccctacatactggttctagactgtgacatgtactgcaatgacccaacctcagctagacaagcaatgtgcttccatcttgatccccaactgtctaaaaatcttgcttttgtacaattccctcaaatattctataacgctagtaagaatgacgtctatgatgcccaagtcagggcggcataccagacaaagtggcagggtatggatggacttcaaggaccaattttttctggcactggcttttacttaaagaggaaggcaatgtatggaaaccctgatcaagatgataattgtctactcaagccatataagaaatttggcatgtctggagaatttgtagaatcacttaaggtccttaacgaacaagatggtacccagaagaaattattggatggatttttacaagaggccaaactattggcctcgtgtgcctatgaaacaaagacaagttggggtaaagagattggattctcatatgactgtttaatagagagcactttcactggttatcttttgcactgcagagggtggatatctgtttatctttatcccaagagaccatgttttttaggatgctgtcctactgatatgaaggatgccatggttcaatataccaagtggatgtctgagctattttcaattgctatctcaagattcaatcctctgctctatggggtggcaagaatgtccattcttcaaagcctgtgttatggatcctttacactggcgcctattttgtcatttcctttgttcttatatggaacggttcctcaattatgcctcttgaaaggcatatctttgtttccaaaggtttcggacccatggtttgctgtgtttgcagctatctttgtatcctccctgtgtcaacactggttcgaggtcctctcttgtgatggtacatttacgacttggtgtaatgaacagcggagttggcttataaagtcggtttccggtagtttgtttggagttgtgggcgcaatcttgcagcggctaggcttgaagacaaagtttagtttatcaaacaaagccatggacaaagaaaagctggagaaatatgaaaagggtaaatttaatttccaaggggctgccatgttcatggttcctgtgtctattttagtcatactgaacacattttgcttcctcggtgggttttggaaagtgatcataatgaagaatatcctggacatgtttggacaactttctctctctgcctacgttctggttctcagttgtccagttcttgaagggatgttaactagaatcagcaagaaaatggtctga

SEQ ID NO: 80 - DNA, 2142 bp, synthetic construct
  source 1..2142: mol_type = other DNA
atgaaatcacccagtaatccaaaccaaaagcctattctacatacgtgtacgattcaacagcccagggcaactcttaataagattcattcattgatacatttttccgcgatccttgttttgttttactatagaataactcgtttgtttttcactgatgatttcaaagtaccaaaattgctatggactttaatgacgatcagcgaattcatcttagctttcatttgggtgctgattcagccttttaggtggaggccagtgtcaagaagcgtaatccccgagaacatgccgaaagatataagcttgcccgctgtagatgtttttgtttgtacagcggatccccagaaagagcctacggtggaggtaatgaatactattctttccgcaatggcgcttgattaccccgcagagaaattagctgtttacttatctgatgacgggggttctgctgtgaccctatacgcaattaaagaagcgtgttgctttgcgaagatgtggttacctttctgtaacaaatatggcatcaaatcaaggtgtccagaagcctactttagtaaattggcggcagatgagtggctacacagaagtgtagagttcgtagccgaagaaaaggaagtaaaggcgaactacgaggagtttaagagaaatgtgcagaagtttggggagcaacaagaaaatagcagagtcgttcacgacaggcatccccacgtcgagatcattcataataactggaacaatgaagaccaggctcacgagatgccgcttttggtctacgtctctagggaaaggcgtccatcacatcacccaagattcaaagcgggggcccttaacaccctgttacgtgttagtggtattatcagcaacagtccctacatcttagtactagactgcgacatgtattgcaacgaccctacatccgcgcgtcaagctatgtgcttccatttagacccgcaactatctaaaaatctggccttcgtgcagtttcctcagattttctataacgccagcaaaaatgacgtgtatgacgcccaggttcgtgcggcatatcagactaaatggcaaggtatggatggtctacaaggccccatcttcagcggcacaggattctatttaaaacgtaaggcgatgtacggtaatccagatcaagatgataactgtctactgaagccatacaaaaagtttggtatgtcaggtgagttcgtcgagagccttaaggtattaaacgaacaggacgggacgcaaaaaaaacttttagacgggtttcttcaggaagccaaactactagccagttgtgcgtacgagaccaagacctcatgggggaaggagatagggttctcttatgactgcctgatcgagtctacctttaccgggtatttattacattgccgtggctggatctctgtgtacctgtaccctaaaagaccttgttttctggggtgttgcccgaccgacatgaaagacgcgatggttcagtatactaaatggatgagcgagctattcagcatagcgatttccagattcaatcctttactatacggcgtggcaagaatgtccatcttacaatcactatgctatggatcattcacacttgcacctatactgtccttcccactatttctttacggcaccgttcctcagctatgtttgttaaaaggcatctcactgttcccgaaagtgtctgacccctggttcgcggttttcgcggcaatctttgtgtcatctctatgtcagcattggtttgaggttctgtcttgtgacggaacttttacgacctggtgcaatgagcaacgttcttggttaattaagagtgtctctggctcactgttcggagttgtaggggcaatacttcagaggttaggtcttaaaacgaagttttctttgtcaaataaagcaatggacaaggaaaaactagagaagtacgagaagggaaagttcaattttcaaggcgcggccatgttcatggtacccgtgtctattttggtcatcctaaatacgttctgctttttaggaggattctggaaagtaattattatgaagaatattctggacatgttcgggcagctttcactatctgcttatgtgttagtactgtcttgcccggttctggagggaatgcttacgagaatatccaagaagatggtttaa

SEQ ID NO: 81 - protein, 707 aa, Quillaja saponaria
MATVSSLHTCTVQQPRAAINRIHIFLHFIAILFLFYYRVTGLFYDNAVPTLAWSLMTLAELIFAFVWVLSQAFRWRPVLRSVIPERIPKDVRLPAVDILICTADPLKEPTVEVMNTVLSAMALDYPAENLAVYLSDDGGSPVTLFAMKQVGPFAKLWLPFCNKYGIKTRHPESFFSAFADDERLHRSDEFRAEEEAIKDKYEEFKRTIEKYGGEGKNSHVVQDRPPHVEIIHDTRKIRENSEDQAVPLLVYVSREKRPSYNSRFKAGALNTLLRVSGVISNSPYVLVLDCDMYCNDPTSARQAMCFHLDPQMSRTLSFVQFPQVFYNVSKNDIYDGQARAAFKTKWQGMDGLRGPLLSGTGFYLKRKSLYGSPNQEDDCLLEPHKNFGKCDKLIESVKVIYERDVSIKADSSDAILQDAKQLASCPYETNTSWGKEVGFSYDCLLESTFTGYLLHCRGWTSVYLYPKKPCFLGCTPVDMKEAMVQYTKWISELFLLAISRFNPLTFGISRMSILQSMCYGYLTIMPILSVAMIFYATVPQLCLLRGVPLFPKVSDPWFAVFLAIFVSSLCQHLIEVLTSDGTLKTWWNEQRNWVIKSGSGSVFGALSGILKWFGMKIKFGLSNKAVDKEKLEKYEKGKFDFQGAAMFMVPLTISVILNTLCLIGGLWRVITLKNFEEMSGQFIISLYFLALSYPILEGLLRKGKGKA

SEQ ID NO: 82 - DNA, 2124 bp, Quillaja saponaria
  source 1..2124: mol_type = genomic DNA
atggcgaccgtctcctccctccacacttgcactgtacagcaaccccgtgcagccattaatcgaattcacattttcttacactttattgccatacttttcctcttttactaccgggtcaccggtcttttctatgacaatgcagtacccactttagcttggtctctaatgaccttagctgagttgattttcgccttcgtttgggtgctcagccaagccttccggtggcgcccggtgttgcgttcagttattcctgagaggattcccaaagatgtacgattgcccgcggtggatatcttaatttgtacggctgacccattaaaggaaccgacggtggaggtgatgaacactgtcttgtccgccatggcattggactatcctgcggagaatctggctgtatatctttctgatgacgggggttctccggtcaccttatttgctatgaagcaagtgggtccgtttgctaagctgtggcttccgttttgcaacaagtacggaatcaaaacaaggcatcctgagtcttttttctcggcatttgcggatgacgaaaggcttcaccggagtgatgaattcagggcagaggaggaggcgatcaaggacaaatatgaagaatttaagagaactatagagaaatatggtggagaaggaaaaaatagtcatgttgtacaagaccggcctcctcatgtggagattatacatgacactaggaagattagagagaacagtgaagaccaagctgtgcctcttcttgtctacgtctctcgtgagaaaagaccatcctacaattctcggttcaaagcaggagctctgaacacccttcttcgagtttctggggtaatcagcaatagcccatatgtattggtgttagactgtgacatgtactgcaatgatccaacatcagctagacaagcaatgtgcttccatcttgatccacaaatgtctcgcactctctcttttgtacaattcccccaggttttctacaatgttagtaaaaatgatatctatgatggccaagctagggcagcctttaagacaaagtggcaaggtatggatggactacgtgggccactgctttctggtactggcttttatttgaagaggaagtccttgtatggaagtccaaaccaagaagatgattgtttacttgagccccataagaattttggaaagtgtgacaagctcatagaatcagtaaaggtcatttatgaacgtgatgtttcaataaaggcagattcatcagatgccattttgcaagatgccaaacaattagcatcttgtccctatgaaacaaacacaagctggggcaaagaggttgggttctcgtatgactgcttattagagagtacattcacaggttatctgttgcactgcagagggtggacatcagtttatctttatccaaagaagccatgtttcttagggtgtactccagttgatatgaaggaagccatggttcagtatacgaagtggatttctgaattatttttacttgctatctcaagattcaaccctctgacatttgggatatccagaatgtccattctccagagcatgtgttacggataccttacaatcatgcccattttatctgttgctatgatcttctatgccacagttcctcaattgtgcctcttgagaggcgtacctctgtttcccaaggtttcagacccatggtttgcagtgttcctagcaatatttgtgtcctccctctgtcagcacttaattgaagtcctcacgagtgatggcacgctcaagacttggtggaatgaacaaagaaattgggtgataaagtctggttccggtagcgtatttggagctctgagtggaatattgaagtggtttggcatgaagattaaatttggtttatcaaacaaagccgtggacaaagaaaagcttgagaaatatgaaaagggtaagtttgatttccaaggggctgccatgtttatggttcccttaactatatcagtcatcttgaacacattatgccttatcggtggtttatggagagtaatcacacttaaaaacttcgaagagatgtcagggcagttcatcatctccttgtactttctagctctcagctatccaattcttgaagggttactaagaaaaggcaagggaaaggcctaa

SEQ ID NO: 83 - DNA, 2124 bp, synthetic construct
  source 1..2124: mol_type = other DNA
atggcgacagtgtcttcattgcatacgtgtactgtccagcagcccagagcggccattaacagaattcacatctttttacattttattgcgatcctgtttttattctattacagggttaccggtctattctacgataatgcggttccaacacttgcatggagtttgatgacacttgcggagttgatcttcgcttttgtctgggtattatcacaagcattccgttggaggcctgtgctaagatcagtgattccggaaaggattccgaaagatgtgagacttccagcagtcgacatcctaatctgtacagcggacccgctaaaggaacctacagtagaagttatgaacacagtcttgagcgccatggcgctagactatcctgccgaaaatctggctgtataccttagcgacgatggcgggagtccggttacccttttcgccatgaaacaggttgggcctttcgcgaagttgtggctgcctttttgcaataagtacggaattaagacaaggcacccagagtcatttttctctgcattcgctgacgacgaacgtttacaccgtagtgacgaattcagagcagaagaagaggccataaaggataagtacgaggagttcaagagaactatcgaaaaatacggtggggaggggaaaaattcccacgtagtccaagatcgtcccccgcacgtggaaattatacatgacacaagaaagatccgtgaaaacagtgaggaccaggctgtgcctctactagtatatgtgtcaagggaaaaacgtccctcatataattcaaggtttaaggctggcgccttaaatacattattacgtgttagtggcgttatcagtaattcaccttacgtgcttgttttggactgcgatatgtactgtaacgatccaacctctgcgaggcaggctatgtgtttccacttagacccccagatgagcaggaccctgagcttcgttcaatttccccaggtcttctataacgtttcaaagaatgatatttacgatggacaagcgagggcggcgttcaaaactaaatggcaagggatggacggcttaagggggccgctgttaagtggaactggtttctatttgaaaaggaaaagcttatatggcagccctaatcaagaagatgattgtttacttgagccccacaagaatttcggaaaatgtgataaactaatagagtcagtgaaagtgatatatgagagggatgtgagtatcaaagccgacagtagcgatgctattttacaggatgctgaacagctggccagctgtccttatgagacaaataccagttggggcaaggaagttggcttctcatatgactgccttcttgaaagcacatttaccggttatttacttcactgtcgtgggtggacctctgtgtacctttacccaaaaaagccatgttttttagggtgcacgcctgtcgacatgaaggaagctatggtgcagtatacaaagtggatatcagaacttttcttgttagcgattagtaggtttaatcctttgacattcggcataagtagaatgagcatactgcaatctatgtgctacgggtacttaaccataatgccgatcctgagtgtagccatgattttctacgctaccgtgcctcagctttgcttgctgagaggagtgccccttttcccaaaggttagcgacccctggttcgcagtatttcttgctattttcgtgagtagcttgtgtcaacatctaatcgaagttttaacgtcagatggtacgttgaagacctggtggaatgagcaaaggaattgggtgatcaaatccggttctggaagtgtgttcggagccctgtcaggaatcctaaaatggtttgggatgaaaataaaattcgggctgtccaataaggccgtcgacaaggaaaaacttgaaaagtacgagaaaggaaagtttgacttccagggagcagcgatgttcatggtccccctaactatatcagtcattcttaacacgttgtgtcttattggcgggttatggagggtcattacgcttaaaaactttgaagaaatgtctggccagtttattatatccctatactttctagcactatcctacccaatactggagggcctactacgtaaaggtaaaggcaaggcgtaa

SEQ ID NO: 84 - protein, 481 aa, Arabidopsis thaliana
MVKICCIGAGYVGGPTMAVMALKCPEIEVVVVDISEPRINAWNSDRLPIYEPGLEDVVKQCRGKNLFFSTDVEKHVFESDIVFVSVNTPTKTQGLGAGKAADLTYWESAARMIADVSKSSKIVVEKSTVPVRTAEAIEKILTHNSKGIEFQILSNPEFLAEGTAIKDLYNPDRVLIGGRDTAAGQKAIKALRDVYAHWVPVEQIICTNLWSAELSKLAANAFLAQRISSVNAMSALCEATGADVTQVAHAVGTDTRIGPKFLNASVGFGGSCFQKDILNLIYICECNGLPEAANYWKQVVKVNDYQKIRFANRVVSSMFNTVSGKKIAILGFAFKKDTGDTRETPAIDVCNRLVADKAKLSIYDPQVLEEQIRRDLSMARFDWDHPVPLQQIKAEGISEQVNVVSDAYEATKDAHGLCVLTEWDEFKSLDFKKIFDNMQKPAFVFDGRNVVDAVKLREIGFIVYSIGKPLDSWLKDMPAVA

SEQ ID NO: 85 - DNA, 1908 bp, Arabidopsis thaliana
  source 1..1908: mol_type = genomic DNA
caaaaaggtccaaattaatctcttcataatctttagctagtaaattttgtaacgaaatgattatggaagcataataattgaaccaaaaagaaaaaaaagcgtgtgggatttattgggagcaataaataaaaagttgaaatgtgtaagtcaagtgggtgcgagtgtatttataaaactttcagaagacatcttctctgcaaattccatcatcgtcgccgcaacaatatcttttgctttctctctcattcttcaccagtaacaagcccatctctcgattcttcactctcaaggaaggagacgttttgaaaatcagaaatggtgaagatatgctgcataggagctggttatgtgggtggtccaaccatggcggtgatggctcttaagtgtcctgagattgaagtagtcgttgtggatatctctgaaccaaggatcaatgcttggaacagtgataggcttcctatttacgagccgggattggaagatgtggtgaaacaatgcagagggaaaaacctcttctttagcacagacgtggagaaacatgtatttgagagtgatattgtatttgtctcagttaacactccaaccaaaacacaaggtcttggtgctggcaaagctgctgatcttacttactgggagagtgctgctcggatgatcgctgatgtctccaaatctagcaaaatcgttgttgagaaatccacggttcctgtgaggacagcagaggctattgaaaagatactgacacataacagcaaaggcatagagtttcagattctctctaaccctgaatttcttgctgagggtactgcaattaaggatctttataacccagaccgtgtgttgattggtggtagggatactgcagcagggcaaaaggctattaaagctttaagagatgtttatgctcattgggttccagtggaacaaatcatttgcacgaacctgtggtccgctgagctctctaagcttgcagcaaatgcattcttagctcagaggatatcatctgtcaatgccatgtcagctctatgtgaggcaactggcgctgatgttacacaagttgcgcatgccgtgggtacagatactagaattggtccaaagttcttgaatgctagtgttggttttggtggatcatgtttccaaaaggacatcctaaatcttatctatatttgtgaatgcaacggcttgcccgaagcagctaattactggaaacaagtcgtaaaggtgaacgactatcagaaaatacggtttgcaaaccgggttgtttcttcaatgtttaacacagtctcgggcaagaaaatcgcgatcctcggttttgccttcaagaaggacacaggtgacacgagagagactccagcgattgatgtttgtaacagattagttgcagacaaggccaagctgagcatatacgacccacaagttcttgaagaacagatcagaagagatctttccatggctaggtttgactgggaccaccctgttcctcttcagcagattaaagctgaaggtatctcagagcaagtgaatgtcgtctcagatgcttacgaggcaactaaagatgcgcacggcctatgtgtcttaaccgaatgggatgagtttaaatccttggacttcaagaaaatctttgacaatatgcagaaaccagcttttgtgttcgatggtaggaatgttgttgatgcagtgaagctgcgtgagatcggtttcatcgtctactccattggtaaaccgcttgattcatggctcaaggatatgcctgctgtggcatgaaggtgccaacttcaaccaaactatatgtcttttctaaaatatagtatcttaagattccttataaattctgttgtaaccaagctgaaacaatgatatgtatttgctgaatctgccttagctggaatagtatatgaatttctttgacta

SEQ ID NO: 86 - DNA, 1446 bp, synthetic construct
  source 1..1446: mol_type = other DNA
atggtgaagatatgctgtatcggggctggatacgtggggggtcccacgatggcggtcatggccctgaagtgtcctgagatagaagttgtggtagtggatataagcgaacctagaataaacgcgtggaactctgacaggttgcctatctacgaaccaggcttggaggacgtcgtcaaacagtgcagggggaaaaaccttttcttttctactgacgtggaaaaacacgtgtttgaatcagacatcgtttttgtcagtgtcaatactcccacaaaaactcagggcctgggagcgggaaaagctgcggacttaacctattgggagtcagccgctaggatgattgcagatgtgtcaaaatcctcaaagatagtagtagaaaaatctaccgtccctgtgcgtactgctgaggctatcgagaaaatacttacgcacaactctaaaggtatagagtttcaaatactgtctaaccctgagttccttgccgaaggtacggctattaaggacctgtacaacccggaccgtgtgcttatcggtggtagggacacagccgctggacaaaaagcgattaaggccttgagggacgtatatgcccactgggtccctgtagaacagataatatgtaccaatctatggtcagcggagttgtcaaagctggccgcgaacgcgttcttagctcagaggatctcatccgttaacgcgatgtcagcgttgtgcgaggcgactggcgccgacgtgactcaagtcgcgcatgcggtaggaacagatacaagaattgggccgaagtttttaaatgcatctgtaggtttcggtggtagttgtttccagaaagatattttaaacttaatttacatttgcgaatgtaatggactgccggaagctgccaattactggaaacaagtagtaaaagtaaacgattatcaaaagataagatttgcgaaccgtgtagtgtcctctatgttcaacactgtaagcggtaagaagatcgccatactgggtttcgcctttaaaaaagatacgggcgatacccgtgaaaccccggccattgatgtttgcaataggctagttgcagacaaggccaaactatccatctatgatccacaagtgcttgaagagcaaattagaagggacttaagtatggctaggtttgactgggatcaccccgtccccctgcaacaaataaaggctgagggcatcagtgaacaggtaaatgtagtatccgacgcatacgaagctactaaggacgcgcatgggctatgcgtgcttacggaatgggatgaatttaagtctcttgattttaagaagatattcgataacatgcagaagcctgcatttgtatttgacgggcgtaacgtggttgatgccgtgaagttgagggagataggctttattgtttacagcattggtaaaccactggactcctggttaaaagacatgcctgcggtagcatag

SEQ ID NO: 87 - protein, 380 aa, Saponaria vaccaria
MRLIFMFFGGKAGKMVDYTPKNILITGAAGFIASHVANRLVRNYPEYNIVVLDKLDYCSNLKNLIPSRSSSKFKFIKGDIASADLVNFILTTEAIDTIMHFAAQTHVDNSFGNSFEFTKNNIYGTHVLLEACKVTGKQIKRFIHVSTDEVYGETDEDAAVGNHETSQLLPTNPYSATKAGAEMLVMAYGRSYGLPVITTRGNNVYGPNQFPEKMIPKFILLAMRGLPLPIHGDGSNVRSYLYCEDVAEAFETILHKGEVGHVYNIGTLKERRVIDVAKEICTLFNRNPDETIKFVENRPFNDQRYFLDDQKLKKLGWSERTSWKEGLIKTMEWYVKNPDWWGDVSGALVPHPRMLMMPGGTDGRINGNGTSITANGNCHS

SEQ ID NO: 88 - DNA, 1143 bp, Saponaria vaccaria
  source 1..1143: mol_type = genomic DNA
atgagattaattttcatgtttttcggtgggaaagcagggaaaatggttgactacacgccaaagaacatactcataacaggagcagctggtttcatagcctcccatgttgcaaacagactggttagaaactaccctgaatacaacatcgtcgttctagacaaacttgactactgctctaacctcaagaatctcatcccgtctcgctcctcttccaagtttaagttcatcaagggagacattgccagtgctgatctggtcaacttcatcctcaccaccgaggccatagacaccattatgcactttgctgcacagacccatgtcgacaactcctttggcaacagctttgagttcaccaagaacaacatctacggcacccacgtccttttagaagcttgcaaggtcacaggaaaacagatcaagaggttcatccatgttagtacagacgaggtttacggggaaacagatgaagatgcagcagtaggcaaccacgagacatctcagctccttcccacaaacccttactctgcaaccaaagcaggagctgaaatgttagtcatggcatatggcaggtcctatggattaccagtgatcaccacgagggggaacaacgtgtatggcccaaaccaattcccggagaaaatgattcccaagttcatacttctggccatgagagggctgcctctcccgattcatggggatgggtcaaatgtgagaagttacttgtactgcgaagatgtggcggaagcttttgagacaatccttcacaaaggggaggtgggccatgtctacaacatcggaactcttaaagagagaagggtcattgatgtggctaaggagatatgcaccttgtttaataggaaccccgatgaaacaatcaagtttgtggaaaacagacctttcaatgatcagaggtactttttagatgaccaaaagctgaagaaattgggttggtccgagaggactagctggaaagagggtttgataaagaccatggaatggtatgtcaaaaatcctgattggtggggagacgtatccggtgccctagtccctcatcctaggatgctcatgatgcctggcggtacagacgggcgtatcaacgggaatggcacttccattactgctaacggcaactgccattcttaa

SEQ ID NO: 89 - DNA, 1143 bp, synthetic construct
  source 1..1143: mol_type = other DNA
atgagactaattttcatgtttttcggaggaaaggccggtaagatggtagattacactccgaagaacattttgatcacgggggctgctggttttatcgcgtctcatgttgcgaatcgtctagtaaggaattacccggaatacaatatcgtagtgctggataaacttgactattgttcaaacttgaaaaacttaattccaagtcgttcatccagtaaatttaagtttatcaaaggcgatatagcttccgcagacttagtcaactttattcttacgacagaggcaattgatactattatgcacttcgcggctcaaacgcacgtggataatagtttcggcaatagctttgagttcactaagaacaatatttacggcacccatgtccttctagaagcgtgtaaagtcactggaaaacaaatcaagaggtttattcatgtttcaacagacgaggtatatggggaaacagacgaagatgccgccgtaggaaatcacgaaacgtcccaactacttccgactaacccttattccgcgacgaaagcaggtgcggaaatgctggtaatggcgtatgggcgttcatacggcttgccagtcataactaccaggggtaacaatgtgtatggccccaaccagttccctgagaaaatgatcccgaaattcatacttctagcaatgagaggcttaccattgcctattcatggcgacggctctaacgtccgttcctatttatattgtgaagatgtcgccgaagcatttgagaccatcctgcataagggtgaagtcggtcatgtatataacatcggcacactaaaagagaggcgtgtcatagatgtcgctaaggagatatgcacgctttttaataggaaccccgacgaaacgattaaatttgtagagaacagacctttcaacgatcagaggtattttttagatgatcagaagctaaaaaagctaggatggtctgagagaacgagctggaaagaaggattaattaagaccatggagtggtatgtcaaaaacccagactggtggggggatgtgtccggagctctagtgccacacccgaggatgcttatgatgccgggcggtactgatggacgtattaacggtaatggaacttccatcactgcgaatggtaactgccacagttaa

SEQ ID NO: 90 - protein, 302 aa, Saponaria vaccaria
MAEAPSFLAQKRYAVVTGANKGLGLEISRQLASQGVVVLLTSRDEKRGFEAIEELKKSGINSENLVYHQLDVTNPSSFASLADFVKAKFGKLDVLVNNAGISGVIVDYPALMEAIRNRGAEINYDGVMKQTYELAEECLQTNYYGVKRTINALLPLLQLSDSPRIVNVSSDVGLLKKIPGERIREALGNAEKLTEESVDGLLDEFLTDFKEGKIAENGWPTFKSAYSISKAALNSYTRVLARTYPSIIINCVCPGVVKTDINLKMGHLTVEEGAVGPLRLALLPPGSPSGLFYMKNEIASFE

SEQ ID NO: 91 - DNA, 909 bp, Saponaria vaccaria
  source 1..909: mol_type = genomic DNA
atggctgaagcaccctcatttcttgctcagaaaaggtatgcggttgtgaccggagcaaacaaaggactaggactagaaatcagcagacagctcgcttcacagggcgtggtggtactgctgacctccagagacgaaaaaagaggctttgaggccattgaggagcttaagaagtctggcataaattcggaaaatcttgtgtatcatcagctcgatgttactaacccgtctagttttgcttctctggctgatttcgtcaaggccaaatttggcaagcttgatgtcctggtgaacaatgctgggatcagcggtgttattgttgattatccagctttgatggaagctattcgcaaccgaggggcggagatcaattacgatggcgtgatgaaacagacctacgagctggcagaggaatgcttgcaaacaaactactatggcgtgaagagaaccattaacgccttacttccactccttcagctatctgattctccaaggatcgtcaatgtttcatccgatgttggcctgcttaagaaaatacccggggagaggattagagaagccttaggcaatgcggaaaagcttacagaagaaagcgtggatgggcttttggacgagtttctaacagattttaaggaaggcaaaatcgcagagaacggttggcctacgtttaagagcgcctattcgatctccaaggcggcactcaattcgtacacaagagttttggctcgcacatatccgtcaatcatcatcaattgtgtctgccctggcgttgtgaaaaccgatatcaaccttaaaatgggccacttgacggtcgaggaaggcgcagtcggtccgttgaggctagcgctcctgcctcccggttcaccgtccggcttgttctatatgaaaaacgagatagcatcatttgaatga

SEQ ID NO: 92 - DNA, 909 bp, synthetic construct
  source 1..909: mol_type = other DNA
atggccgaggctccctccttcctagcccagaaaaggtatgccgtggttacaggagcaaacaagggcttgggtctagaaatctcacgtcaactagcctctcagggggtcgttgtactattaaccagtcgtgatgaaaagcgtggattcgaagccatagaagaactaaaaaagagcggcataaactcagaaaatttagtatatcatcagctagatgtaacgaatccgagcagtttcgccagtttagctgatttcgtaaaggctaagtttggcaagttagatgtgctagttaataacgcggggatatccggcgtcattgtggactatcctgccctaatggaagcgataaggaaccgtggcgcagaaataaactatgacggggtaatgaagcagacgtatgaactggctgaggaatgcctacagacaaactattacggtgtgaaaagaacaatcaatgcactgttacctcttctacagctatcagattcccccaggatcgttaacgtcagtagtgatgttggactgttgaagaaaatcccgggtgagaggataagggaagcattaggcaacgcagaaaagctaactgaggagtctgtggatggtttgctggatgagttcttgactgattttaaggagggcaagattgccgagaacggttggcctacgttcaagtcagcatatagtatttctaaagcagcacttaactcttacacacgtgtattagcaagaacatacccgagtatcattataaattgcgtttgcccgggtgttgttaaaacagacataaacctgaaaatgggacacttgactgtggaagaaggagcggttggccccctgaggcttgcactactgccacctggaagtccgtccgggcttttttacatgaagaatgaaattgcctccttcgagtga

SEQ ID NO: 93 - protein, 457 aa, Quillaja saponaria
MENGRVYKSHVVVLAFHGQGHIVPLIQLSRRLAWKGIKITFATTHSCTKAIQTGSDSISLLSIYDDITDGGFQGEGGFKGFLQRFEASTTRILHEFVKNHENSKNPVKCLIYDANLIWALEMAKQLGIATAAFVFPSWAAIATYYPFYLEVYADQQIKKVDPFTMPDLPPQLGLPNMASLGSDSGQHSPILKLMLQQLENFGKADWILSHAFEQFEQEVLDWMRNISPVTTIGPTLPSVYLDGRLKDDTDYGYNLYKPDSDTCMKWLDTKETESVVYISFGSVADLIPEQMTEITNSLKKMSSNFLWVVKETEKNNLPSSFVEETKEKGLVVTWCPQLKVLSHPAVGCFITHCGTNSIFESVCFAVPMVGMPQFCDQMPNAYFMEKVWKVGVRPSLDDNGVVTGEEIERCIKVVTEGESGQEIKKKLVQWKELAKEAVDEGGSSDKHIDEFIAGITT

SEQ ID NO: 94 - DNA, 1374 bp, Quillaja saponaria
  source 1..1374: mol_type = genomic DNA
atggagaatgggagagtttacaaatcccatgtcgtggtgctcgcatttcacgggcaaggccatattgttccgttaatccaattatccagacgattggcctggaaaggcatcaaaatcacatttgctacaacacattcttgcaccaaggccattcaaacaggaagtgattcaatttcacttttatcaatttatgatgacataactgatggtgggtttcaaggagaaggaggattcaagggcttccttcagagatttgaagccagtaccacaaggatcttacacgaattcgtcaagaatcatgaaaactcaaagaacccagtaaaatgcttaatatatgatgctaacttaatatgggctctggaaatggcaaagcaattgggtattgctactgctgcatttgtgtttccttcttgggctgccattgccacctactatcccttttatttagaggtgtatgcggatcagcagataaagaaggtagatcctttcacaatgcctgacttacctccacaacttggacttccaaatatggcatctctcggttcagattcgggtcaacactcccccatactcaaactcatgttgcaacagttagaaaattttgggaaagctgactggatcctgtctcacgcatttgaacagtttgaacaagaggtacttgactggatgagaaatatcagcccagtaacaacaattggtccaactctgccatctgtttatcttgatggtaggctaaaagatgacacagattacggttacaatttgtacaagccagatagtgatacctgcatgaagtggctagacactaaggaaactgaatcagtggtttatatatcatttggcagtgttgcagatttgatcccagaacagatgacagaaataacaaactccctgaagaaaatgagcagcaactttctgtgggtggtgaaggaaactgaaaaaaacaacctccctagcagctttgttgaggagacaaaagaaaagggattggtagtgacttggtgcccccagttgaaggtgttgtctcatcctgcagtgggttgtttcattacacactgtggaacaaattccatatttgagtcagtatgctttgcagtgccaatggtgggaatgccacagttttgtgatcaaatgcctaatgcatatttcatggagaaggtctggaaagtaggtgttaggccaagtttggatgacaatggtgtcgtcactggagaagaaattgagcgatgtataaaagtagttaccgaaggagagagtgggcaagagattaagaagaaacttgtgcagtggaaagagcttgcaaaagaggcagtggacgagggtggaagttcagataagcacattgatgaattcattgctggaatcacaacttga

SEQ ID NO: 95 - DNA, 1380 bp, synthetic construct
  source 1..1380: mol_type = other DNA
atggagatggagaacggcagggtctataaaagccacgtcgtggtgttagcctttcacggtcagggtcatatagtgccgctgattcagctttctagaagactggcttggaagggcatcaagataacgttcgcaacgacacattcatgcacaaaagcaatacaaacggggtctgattctattagcctactttccatttacgacgatataactgatggtggcttccaaggggaagggggctttaagggctttctacaacgtttcgaggcttcaacgaccaggatattgcatgaatttgttaaaaaccacgagaacagcaaaaaccctgtgaaatgcttgatttacgacgcaaacttgatttgggcgctggaaatggcaaagcagttggggattgccacggcggcattcgtattcccgagttgggcggcaattgcgacctactatccgttctacttagaagtctacgccgaccaacaaataaaaaaggtggaccccttcacgatgccggacctgcctccacaattgggcctaccgaatatggcctcacttggttctgactccggccaacactctcctatcctaaagttgatgcttcagcaacttgaaaacttcgggaaagctgactggatcttgagccacgcattcgagcaattcgagcaggaggtcctggactggatgcgtaacatctctccggtgacaactattggtcccacgttacccagtgtgtatctagacggaaggttaaaggacgatactgattacggatataatctttataaacccgatagtgatacttgtatgaagtggcttgatactaaggagactgaaagcgtagtgtatatttcatttggtagcgtagccgacctaatccccgagcagatgactgaaattacaaatagtcttaaaaaaatgagtagcaactttctttgggtcgtgaaggagactgaaaaaaacaatctacccagctcatttgtggaagaaaccaaggagaagggattggtcgtaacgtggtgtcctcaactgaaagtgttgtcccacccagctgtggggtgctttattacgcattgcgggacgaacagtatatttgagagtgtctgtttcgcagtgccaatggttggaatgccccaattttgtgaccaaatgccaaacgcgtactttatggaaaaagtgtggaaagttggcgtccgtccctctcttgatgacaacggtgtagtgaccggtgaagaaatcgagcgttgcatcaaggtcgttaccgagggtgagtccggtcaagaaatcaaaaagaagctggtacagtggaaggaattggcgaaagaagcagtcgacgaaggcggttcttcagataaacatatagacgaattcatagcagggattacaacttga

SEQ ID NO: 96 - protein, 473 aa, Saponaria vaccaria
MAHEIKKIEIIVFPYHGQGHMNTMLQFAKRLAWKNVVNVTIATTLGTTNKMRSQVDQVDINSAWAASIDLVSVYDDTDESQVRFMDRMARFEAAAASNLATLLVKKKEHADNKVLLVYDGNLPWALDVAHGKGVRGAAFFPQSCATVATYYCLYNETQGREVEPKLPDVFPPLELIQRNIPNVYGMKFPEAVVPKNGKEYSDFVLYVLRQCVNLEKSDLVLFNQFDKLVEPGEVLQWMSKIFNVKTIGPTLPSAYIDKRIKDDVAYGFHAFNLDNDTCMNWLSTKPAGSVIYIAFGSSVHYSVEQMTEIAEALRSQPKNFLWAVRETEQVKLPADFIEQTADKGLVLAWCPQLDVLKHESIGCFVTHCGWNSITEALSFGVPMLSVPQFLDQPVDAHFVEQVWGAGVTVKRNEDGFVTRDEIIRCLDELNDGERAAEIKANVARWKVLAKEALEEGGSSDKHIDEIIEWVSSF

SEQ ID NO: 97 - DNA, 1422 bp, Saponaria vaccaria
  source 1..1422: mol_type = genomic DNA
atggcgcatgaaataaagaaaatcgaaataatagtgttcccgtaccatggacagggacacatgaacaccatgctacagttcgccaagcgacttgcatggaaaaacgtcgtcaacgtcaccattgccaccacgttgggtactaccaacaaaatgcggtcccaggtggaccaggttgatatcaactctgcgtgggcagcttccattgacctggtgtctgtgtacgatgacacagacgagtcccaggtcaggttcatggaccgcatggccaggttcgaggctgctgctgcgtccaacctggccacgctcctagttaagaaaaaggagcatgctgataacaaggtcctgttggtttacgatgggaacctgccgtgggcgctggatgtcgcccacgggaaaggggtccgtggggccgccttcttcccgcagtcgtgtgccactgtggctacgtactactgtctgtataatgagacacaggggagggaggtggagccgaaattgccagacgtgttcccgccgttggagctgatacagaggaatattccgaatgtgtatgggatgaagttcccggaggcggtggtgccgaagaatgggaaggagtatagtgactttgtgttgtatgtgttgaggcagtgtgttaatcttgagaaatctgatttggtgctttttaatcagtttgataagttggttgaacctggagaggttctccaatggatgtccaaaatattcaatgtgaagacaatcggaccgacacttccatctgcatacatcgacaaaagaattaaagacgatgtggcctacggtttccacgcattcaaccttgacaatgacacctgcatgaactggctgagcaccaaacctgctggctctgtcatctacatagcattcggaagcagcgtccactacagcgttgagcaaatgaccgaaatagccgaggccttgaggagccaaccgaaaaacttcctgtgggcagtcagggaaaccgaacaggtgaaactccctgcagacttcatagaacagaccgcagacaaagggctagttctcgcctggtgccctcaacttgatgtcttgaagcatgaatctatcggctgtttcgtgacccattgtggttggaattcgatcactgaggcattgagcttcggggtgccgatgctgtcagtgccacagtttctagaccagcctgtggatgctcactttgtggagcaggtttggggtgcaggagtcacggtcaagaggaacgaagacggtttcgttactcgagatgaaatcattcgttgcttggacgagttgaacgatggtgaacgagcggcagaaatcaaggcgaatgtagcgaggtggaaggttttggctaaggaggcgttggaagaaggcggtagttctgataagcacattgatgaaattattgaatgggtttcatccttctaa

SEQ ID NO: 98 - protein, 489 aa, Saponaria vaccaria
MGSLTEPTKVPVKMVFLTLPIAGHMLHIVDTASTFAIHGVECTLITTPANVPFIEKSLPKSNERILQFFKLGLVAFPHEAVGLPPGLENFSAVTSPDQRGKISKAMSLLQGPTEDLIKEISPDCIVSDMFYPWTSDFALGIGIPRVVFRGCGMFPMACWHSIKSHSPHEGVDCQEDHVIVLPGLPHHIEMRKSTLPDWVRKPSGYTYMMNMMDAAEMRSYGVIVNSFSELEREYEEYYRSVTGLKVWTIGPISLHVGRNEELDGSDEWVKWLDSKQIGSVLYVSFGGVAKFPPHQLREIAAGLESSGRDFVWVVRASEEHASETADAWSLQEFKERMRESKQGLIIESWVPQLMFLEHKATGGMLTHVGWGTMLEGITAGLPLVTWPLYAEQLYNERLVVDVLKIGVGVGVKEFSGLDDIGKKETIGRESIEASVRLVMGDGEEACAMRRRVKELSEASKKAVQEGGSSKANIQDFLNDLSRLRSLRNA

SEQ ID NO: 99 - DNA, 1470 bp, Saponaria vaccaria
  source 1..1470: mol_type = genomic DNA
atgggttcacttacagaaccaacaaaggtgccggtgaaaatggtgttccttacgctcccgatagcgggccacatgctccacatcgtggacacggcgagcacattcgccatccacggtgtggaatgcaccttaataacgacccctgccaatgtccccttcattgaaaaatcccttccaaagtcgaacgaacgaatcctccaatttttcaaactcggcctcgtggcgttcccccacgaggccgtgggcctcccccctggcctggagaatttcagcgcggtcacgtctcccgaccagagggggaagatatccaaggccatgtccttgttgcaggggcccacagaggacctcatcaaggaaatatctcccgactgtatcgtctcggatatgttttacccctggacctcggactttgccctcgggattggcatcccgagggtcgtgttccgaggctgcgggatgttccccatggcgtgctggcatagcattaagtcccactccccccatgagggggttgactgccaggaggatcatgtcatcgttctccctggactcccgcatcatattgagatgcgcaagtctacgttgcccgactgggttaggaaacccagcgggtatacgtatatgatgaacatgatggatgccgccgaaatgaggagttacggagtgattgttaatagttttagtgagctcgagagggagtatgaggagtattataggagtgtgaccgggttaaaggtgtggactatcggtcccatttcgttgcacgtgggccggaatgaggagctagacgggtcggatgagtgggttaagtggttggattcgaaacaaattgggtcggttttgtatgtgagttttggtggggtggccaagtttccaccccaccagctgagggaaatcgcggctgggttggaatcgtctggccgcgatttcgtttgggtggtgagggcgagtgaggagcacgcgtctgaaacagcagacgcgtggtccttacaggagtttaaggagaggatgagggagagtaagcaagggttgattattgagagttgggtcccacagcttatgtttttggaacataaggcgacgggtgggatgctgacgcatgtcggttgggggacgatgttggaagggatcacggcggggttgccgttggtgacgtggccgttgtatgccgagcagctttacaatgagaggttggttgttgatgtgttgaagattggggttggtgttggggtgaaggagtttagtgggttggatgatattgggaagaaggagacgattggtcgggagagtatcgaggcgtctgtgagactcgtgatgggggatggcgaggaggcgtgtgccatgagacggcgggtgaaggagctgagtgaggcttctaagaaggctgttcaggaaggcggttcgtctaaggctaatatacaagattttcttaacgacttgtctaggttgagatccctaaggaatgcttga

SEQ ID NO: 100 - protein, 489 aa, Saponaria vaccaria
MKSPLKMYFLPYISPGHMIPLSEMARLFANQGHNVTIITTTANATLLDKYTTSNLSLHLIPLPTKEAGLPDGLENFISVNDLETAGKLYYALSLVQPVIEEFITSSPPDCIVSDMFYPWTADLASRLQVPRMVFHAACIFAMCMKECMRGPEAPHFEVSSDYELFEVKGLPDPVFMTRAQLPDYVRTPNGYTQLMELWREVERKSYGVMVNNFYELDAAYTEHYSKIMGHKVWNIGPAAQILHRDSGDKIERVHKAVVSEHECLAWLDTKEANSVFYVCFGSAIRFPDDQLYEIASALESSGAQFIWAVLGKDNVGSEPGSGSDSKWLPVGFEEKMRETGRGMIIRGWAPQVLILDHPAVGGFMTHCGWNSTIEGVSAGVGMVTWPLYAEQFYNEKLITQVLKIGVPAGVEEWNMWVDVGRKLVKREKIEEAIRAVMGDEGVEMRKKAKELSAKAKAAVQDGGSSHHNLIALIEDLQRIRDEKLSNVVN

SEQ ID NO: 101 - DNA, 1470 bp, Saponaria vaccaria
  source 1..1470: mol_type = genomic DNA
atgaagtcgccactgaaaatgtacttcttgccctacatttcaccaggccatatgataccgctttccgaaatggcgaggttgtttgccaaccagggccacaacgtcaccatcatcaccaccaccgctaatgccacgcttctcgacaagtacactacgtccaacctttcactacatctcattcccctgcctactaaagaggccggcctaccggacggcctcgagaattttatttctgtcaatgacctcgagaccgcggggaaactctactacgcgctgtcgttagtgcagcctgttatcgaggagtttatcacgtcgagcccgcctgattgtatcgtgtccgatatgttctacccgtggaccgcggacctggcgtcacgactccaggtcccgcggatggtttttcatgccgcgtgtatattcgctatgtgtatgaaggagtgtatgcggggccctgaggccccgcattttgaggtcagctctgattatgagctgtttgaagttaaggggctgccggaccctgtttttatgacccgggcccagctccccgactatgttcgtaccccgaacgggtacacacagcttatggagctgtggcgggaagttgaaagaaaaagttacggtgttatggttaataatttttacgaacttgacgcggcttataccgaacattatagtaaaattatgggccataaggtgtggaatattgggcccgcggcccagatattacaccgtgattctggcgataaaattgagcgagttcacaaagcggttgttagcgagcacgagtgcttagcttggcttgacaccaaggaagctaactcggttttctacgtttgttttgggagcgctattaggttcccagatgaccagctgtacgagatagctagcgcgctagagtcatctggcgcgcagttcatctgggccgttctcggcaaagataatgtaggttcggagccagggtcaggctcggactcaaaatggttgccggtagggtttgaggagaaaatgagggagacggggagagggatgataatacgaggctgggccccgcaggtgttgatattggaccacccggctgtaggcgggttcatgacgcactgcggatggaactcgacaattgaaggggttagcgcgggggtggggatggtgacgtggccgttgtacgcggagcaattctacaatgagaagttgattacgcaagtgctcaagataggggtgcctgcgggggtggaggagtggaacatgtgggtggatgttgggaggaagttggtgaagcgggagaagatcgaggaggcgataagggccgtcatgggggacgagggggtggagatgaggaagaaggctaaagaattgagtgctaaggctaaggccgcggtgcaggatggtggatcgtctcaccataatttgatcgctttgattgaagatcttcagaggattagagatgaaaaactaagtaatgttgttaactag

SEQ ID NO: 102 - protein, 667 aa, Arabidopsis thaliana
MDDTTYKPKNILITGAAGFIASHVANRLIRNYPDYKIVVLDKLDYCSDLKNLDPSFSSPNFKFVKGDIASDDLVNYLLITENIDTIMHFAAQTHVDNSFGNSFEFTKNNIYGTHVLLEACKVTGQIRRFIHVSTDEVYGETDEDAAVGNHEASQLLPTNPYSATKAGAEMLVMAYGRSYGLPVITTRGNNVYGPNQFPEKMIPKFILLAMSGKPLPIHGDGSNVRSYLYCEDVAEAFEVVLHKGEIGHVYNVGTKRERRVIDVARDICKLFGKDPESSIQFVENRPFNDQRYFLDDQKLKKLGWQERTNWEDGLKKTMDWYTQNPEWWGDVSGALLPHPRMLMMPGGRLSDGSSEKKDVSSNTVQTFTVVTPKNGDSGDKASLKFLIYGKTGWLGGLLGKLCEKQGITYEYGKGRLEDRASLVADIRSIKPTHVFNAAGLTGRPNVDWCESHKPETIRVNVAGTLTLADVCRENDLLMMNFATGCIFEYDATHPEGSGIGFKEEDKPNFFGSFYSKTKAMVEELLREFDNVCTLRVRMPISSDLNNPRNFITKISRYNKVVDIPNSMTVLDELLPISIEMAKRNLRGIWNFTNPGVVSHNEILEMYKNYIEPGFKWSNFTVEEQAKVIVAARSNNEMDGSKLSKEFPEMLSIKESLLKYVFEPNKRT

SEQ ID NO: 103 - DNA, 2526 bp, Arabidopsis thaliana
  source 1..2526: mol_type = genomic DNA
gagtttaatgtcggacgctagactcacgagaaatccgtaatatctaagacgatcaagggtaaattagtcaatttcctcctacggttctccgtaactgtctcagaatataagaggcacattgctgagagaagctccgtagaccgtagattccctcttcttcttcttcctcttcctctctactgttttttcttttcatatttctctctctctctctctctatagttggtgaagtttctatctttatctctcttaagatctctttctctcttgggttatctgactctaaccagaattgtgtcgatttcaaggatggatgatactacgtataagccaaagaacattctcattactggagctgctggatttattgcttctcatgttgccaacagattaatccgtaactatcctgattacaagatcgttgttcttgacaagcttgattactgttcagatctgaagaatcttgatccttctttttcttcaccaaatttcaagtttgtcaaaggagatatcgcgagtgatgatctcgttaactaccttctcatcactgaaaacattgatacgataatgcattttgctgctcaaactcatgttgataactcttttggtaatagctttgagtttaccaagaacaatatttatggtactcatgttcttttggaagcctgtaaagttacaggacagatcaggaggtttatccatgtgagtaccgatgaagtctatggagaaaccgatgaggatgctgctgtaggaaaccatgaagcttctcagctgttaccgacgaatccttactctgcaactaaggctggtgctgagatgcttgtgatggcttatggtagatcatatggattgcctgttattacgactcgcgggaacaatgtttatgggcctaaccagtttcctgaaaaaatgattcctaagttcatcttgttggctatgagtgggaagccgcttcccatccatggagatggatctaatgtccggagttacttgtactgcgaagacgttgctgaggcttttgaggttgttcttcacaaaggagaaatcggtcatgtctacaatgtcggcacaaaaagagaaaggagagtgatcgatgtggctagagacatctgcaaacttttcgggaaagaccctgagtcaagcattcagtttgtggagaaccggccctttaatgatcaaaggtacttccttgatgatcagaagctgaagaaattggggtggcaagagcgaacaaattgggaagatggattgaagaagacaatggactggtacactcagaatcctgagtggtggggtgatgtttctggagctttgcttcctcatccgagaatgcttatgatgcccggtggaagactttctgatggatctagtgagaagaaagacgtttcaagcaacacggtccagacatttacggttgtaacacctaagaatggtgattctggtgacaaagcttcgttgaagtttttgatctatggtaagactggttggcttggtggtcttctagggaaactatgtgagaagcaagggattacatatgagtatgggaaaggacgtctggaggatagagcttctcttgtggcggatattcgtagcatcaaacctactcatgtgtttaatgctgctggtttaactggcagacccaacgttgactggtgtgaatctcacaaaccagagaccattcgtgtaaatgtcgcaggtactttgactctagctgatgtttgcagagagaatgatctcttgatgatgaacttcgccaccggttgcatctttgagtatgacgctacacatcctgagggttcgggtataggtttcaaggaagaagacaagccaaatttctttggttctttctactcgaaaaccaaagccatggttgaggagctcttgagagaatttgacaatgtatgtaccttgagagtccggatgccaatctcctcagacctaaacaacccgagaaacttcatcacgaagatctcgcgctacaacaaagtggtggacatcccgaacagcatgaccgtactagacgagcttctcccaatctctatcgagatggcgaagagaaacctaagaggcatatggaatttcaccaacccaggggtggtgagccacaacgagatattggagatgtacaagaattacatcgagccaggttttaaatggtccaacttcacagtggaagaacaagcaaaggtcattgttgctgctcgaagcaacaacgaaatggatggatctaaactaagcaaggagttcccagagatgctctccatcaaagagtcactgctcaaatacgtctttgaaccaaacaagagaacctaagccttcaatgaacatcacactcttccacaagtgcttactttaaactgttttgttttgtttttttttctttttgccttttcttccaacagatatatcttttttatatttactcatcgtcttatgatccttgtaggtagtttcactatattgattctactaaaatatgatttacgattattaatatttatatagacgagagtttttcttttcctc

SEQ ID NO: 104 - DNA, 2004 bp, synthetic construct
  source 1..2004: mol_type = other DNA
atggatgacacgacttataagccgaaaaatatacttattacaggagctgctggtttcattgcatcccacgtggctaacaggcttattagaaattacccggattataagattgtggtacttgataagttagactattgttccgacctaaagaacttggatccatctttctcctctcccaacttcaagttcgttaagggggacatcgcttcagatgatctagtgaactatttgctgataacggaaaacattgacactattatgcactttgcagcacaaacacacgtggataattcatttggcaacagtttcgaattcacgaagaataatatctacgggacacacgtactgcttgaagcgtgtaaagttaccggccagatccgtaggtttattcatgtgagtactgatgaagtctacggtgaaaccgatgaagatgccgccgtaggaaatcacgaggcttcccagttactaccaacgaatccctactctgctactaaagctggtgcagaaatgctagtgatggcatacggccgttcatatggattacctgtcattaccactagagggaacaacgtttatggccctaaccagtttccagagaagatgattccaaagtttatcctgttggctatgagtgggaagccgttacctatccacggcgatgggtcaaacgtaagatcttatctttactgtgaagacgtcgccgaggcttttgaagtggtactacataagggtgaaataggccacgtttataacgtaggcaccaagagagagaggcgtgttatcgatgtcgcgagagacatatgtaagttgttcggaaaggacccggagtcatccatacagtttgttgagaacaggccctttaacgaccagaggtatttcctggacgatcagaaactgaaaaagctaggttggcaagaacgtaccaactgggaggatgggctgaagaagaccatggattggtacacccaaaatcccgagtggtggggtgatgtgtctggagcacttctgccacatcccagaatgctaatgatgccgggaggaaggttgagcgacggtagctccgaaaaaaaagatgtatcttccaacacagtccaaaccttcacagttgtgaccccaaagaacggcgatagtggcgataaagcctctctgaaattccttatttatggtaaaacgggttggctagggggtctgctaggcaaactatgcgaaaaacaagggataacgtacgagtacggaaagggtaggttagaggatcgtgcatcccttgttgcggacataagatccattaagcctactcacgttttcaacgccgccgggcttacaggaaggcctaacgtcgattggtgcgaatcacacaagcctgagacgataagggtcaacgtagcggggactctaaccctagccgatgtgtgcagagaaaatgacttgttaatgatgaattttgccaccggctgtatctttgaatatgacgctactcatccagaaggctccggtatcgggttcaaggaagaggacaagcctaacttctttggcagtttttattcaaagacaaaagccatggtcgaagaactactgcgtgaatttgacaatgtatgtactttaagggtgagaatgccgatatcaagcgacctgaataatcccagaaactttattactaaaatttcccgttataacaaagttgtggatataccaaactcaatgactgtactagacgagttattacctatatctattgagatggctaagaggaacttaaggggcatttggaacttcacaaatccgggcgtcgtcagtcataatgagatacttgaaatgtataagaattacattgagccggggttcaagtggtccaacttcactgtagaagaacaagctaaagtcatagtggctgctagatctaacaacgagatggatggaagcaagctatccaaggagttccccgagatgctaagtataaaggaatccttattaaagtatgtttttgagcctaacaaaaggacgtaa

SEQ ID NO: 105 - protein, 342 aa, Arabidopsis thaliana
MAATSEKQNTTKPPPSPSPLRNSKFCQPNMRILISGGAGFIGSHLVDKLMENEKNEVVVADNYFTGSKENLKKWIGHPRFELIRHDVTEPLLIEVDRIYHLACPASPIFYKYNPVKTIKTNVIGTLNMLGLAKRVGARILLTSTSEVYGDPLIHPQPESYWGNVNPIGVRSCYDEGKRVAETLMFDYHRQHGIEIRIARIFNTYGPRMNIDDGRVVSNFIAQALRGEALTVQKPGTQTRSFCYVSDMVDGLIRLMEGNDTGPINIGNPGEFTMVELAETVKELINPSIEIKMVENTPDDPRQRKPDISKAKEVLGWEPKVKLREGLPLMEEDFRLRLNVPRN

SEQ ID NO: 106 - DNA, 1358 bp, Arabidopsis thaliana
  source 1..1358: mol_type = genomic DNA
atgacatttaatgcgtactcaggattgagatctctctctcaagcaatggcagctacaagtgagaaacagaacaccacaaagcctcctccttctccttctcctctccgcaattccaagttttgtcagcccaatatgaggatcttgatctctggaggagctggcttcattggttctcacttggttgataagcttatggaaaatgagaagaatgaggtggttgttgctgataactatttcactggctcaaaagaaaacctcaagaagtggatcggtcaccccaggtttgaacttattcgtcacgatgttaccgagcctttgttgatcgaggttgatcggatttaccatcttgcttgtcctgcctctcctatcttctacaaatacaaccctgttaagacaatcaagaccaatgtgattggtacactcaacatgctcggtcttgccaagcgtgttggagcaagaattttactaacctcaacctctgaagtgtatggagatcctctcatccaccctcaaccagagagctactggggaaatgtcaaccctattggggttcggagttgctatgacgaaggcaagcgggtagccgaaaccttgatgtttgactaccacagacaacatggcattgaaatccgcattgctagaatcttcaacacatatggtcctcgaatgaacatcgatgatgggcgtgttgtgagcaacttcattgctcaagcactccggggtgaggcattgacagttcagaaaccggggacacagacccgcagtttctgttatgtctccgacatggtggatggacttatccgtcttatggaaggcaatgatactggccctatcaacatcggtaacccaggtgagttcacaatggtggaactggctgagacggttaaggagcttattaacccaagcatagagataaagatggtggagaacacaccagatgatccaagacagaggaaaccagacattagtaaagccaaagaagtgttgggttgggagccaaaggtgaagctcagagaaggacttcctctcatggaagaagatttccgactaaggcttaacgtcccaagaaactaaaaccacaagaaaacacagacggttttgagttcacgagagttgtcagctgttgagttatatgatgatgctattatactacaaagtggtgggtttttgtttggtctcatgtgttattcatattgcttttgttcgtagaaaaataagttgggaatgggtctccttgtgaggaatggctcttctttgtttttcagtatattgtggtatggttattatttgtgtggttttaatgaaatttgttcacttaatttgctcatggaattacagttttaaaatcgttttacagt

SEQ ID NO: 107 - DNA, 1029 bp, synthetic construct
  source 1..1029: mol_type = other DNA
atggcggcaacaagtgaaaaacagaacactaccaagccgccgccttcaccctcacctctaagaaactccaaattctgccaacccaatatgagaatactaatcagcggtggggccgggtttattggttcccatcttgtggataaattgatggaaaacgagaagaacgaggtcgtcgtagcggacaattatttcacgggttctaaagaaaatttaaagaaatggataggtcatcctagattcgagttaatacgtcatgatgtaacagaaccacttctaatcgaggtcgaccgtatctatcacctggcatgcccagcgtctcccatcttttataagtacaacccggtcaagacgataaagaccaacgtaatcggtacactgaatatgctagggttagcaaaacgtgtgggtgcaaggattttgcttacctctacctccgaggtatatggcgacccgttaatacacccacaacctgagagttattggggaaatgtaaatccaatcggagtgagatcatgttatgacgaggggaagagagtcgccgaaacgcttatgtttgattaccacaggcagcacggaatcgaaatcagaatagcaaggatattcaatacttatggccccagaatgaacattgacgatggaagagtggtttccaactttattgcacaagcgctaaggggggaagctctaaccgtccaaaagccaggcacgcagacaagaagtttttgttacgtcagtgatatggtggatgggcttatccgtctaatggagggaaacgacaccggtccgattaatatcggcaaccctggagagtttactatggtggaattagcagaaaccgttaaggaactgataaacccctctattgaaatcaaaatggtcgagaatactcccgacgatccaaggcagaggaagccggacatatctaaagcgaaagaagtgcttggctgggaacccaaagttaagctgagagagggactgcccctgatggaggaggactttaggcttagacttaatgtgccaaggaactaa

SEQ ID NO: 108 - protein, 481 aa, synthetic construct
MVKICCIGAGYVGGPTMAVMALKCPEIEVVVVDISEPRINAWNSDRLPIYEPGLEDVVKQCRGKNLFFSTDVEKHVFESDIVFVSVNTPTKTQGLGAGKALDLTYWESAARMIADVSKSSKIVVEKSTVPVRTAEAIEKILTHNSKGIEFQILSNPEFLAEGTAIKDLYNPDRVLIGGRDTAAGQKAIKALRDVYAHWVPVEQIICTNLWSAELSKLAANAFLAQRISSVNAMSALCEATGADVTQVAHAVGTDTRIGPKFLNASVGFGGSCFQKDILNLIYICECNGLPEAANYWKQVVKVNDYQKIRFANRVVSSMFNTVSGKKIAILGFAFKKDTGDTRETPAIDVCNRLVADKAKLSIYDPQVLEEQIRRDLSMARFDWDHPVPLQQIKAEGISEQVNVVSDAYEATKDAHGLCVLTEWDEFKSLDFKKIFDNMQKPAFVFDGRNVVDAVKLREIGFIVYSIGKPLDSWLKDMPAVA

SEQ ID NO: 109 - DNA, 1446 bp, synthetic construct
  source 1..1446: mol_type = other DNA
atggtgaagatatgctgtatcggggctggatacgtggggggtcccacgatggcggtcatggccctgaagtgtcctgagatagaagttgtggtagtggatataagcgaacctagaataaacgcgtggaactctgacaggttgcctatctacgaaccaggcttggaggacgtcgtcaaacagtgcagggggaaaaaccttttcttttctactgacgtggaaaaacacgtgtttgaatcagacatcgtttttgtcagtgtcaatactcccacaaaaactcagggcctgggagcgggaaaagctctggacttaacctattgggagtcagccgctaggatgattgcagatgtgtcaaaatcctcaaagatagtagtagaaaaatctaccgtccctgtgcgtactgctgaggctatcgagaaaatacttacgcacaactctaaaggtatagagtttcaaatactgtctaaccctgagttccttgccgaaggtacggctattaaggacctgtacaacccggaccgtgtgcttatcggtggtagggacacagccgctggacaaaaagcgattaaggccttgagggacgtatatgcccactgggtccctgtagaacagataatatgtaccaatctatggtcagcggagttgtcaaagctggccgcgaacgcgttcttagctcagaggatctcatccgttaacgcgatgtcagcgttgtgcgaggcgactggcgccgacgtgactcaagtcgcgcatgcggtaggaacagatacaagaattgggccgaagtttttaaatgcatctgtaggtttcggtggtagttgtttccagaaagatattttaaacttaatttacatttgcgaatgtaatggactgccggaagctgccaattactggaaacaagtagtaaaagtaaacgattatcaaaagataagatttgcgaaccgtgtagtgtcctctatgttcaacactgtaagcggtaagaagatcgccatactgggtttcgcctttaaaaaagatacgggcgatacccgtgaaaccccggccattgatgtttgcaataggctagttgcagacaaggccaaactatccatctatgatccacaagtgcttgaagagcaaattagaagggacttaagtatggctaggtttgactgggatcaccccgtccccctgcaacaaataaaggctgagggcatcagtgaacaggtaaatgtagtatccgacgcatacgaagctactaaggacgcgcatgggctatgcgtgcttacggaatgggatgaatttaagtctcttgattttaagaagatattcgataacatgcagaagcctgcatttgtatttgacgggcgtaacgtggttgatgccgtgaagttgagggagataggctttattgtttacagcattggtaaaccactggactcctggttaaaagacatgcctgcggtagcatag

SEQ ID NO: 110 - protein, 453 aa, Paramoeba atlantica
MDVETKEVVCIGAGYVGGPTMTVMAKHCPEINFHVVDINEKRINEWNSEILPIYEPGLDEMVKQVRGRNLFFSTNVKEAIAKGDIIFVSVNTSTKEYGLDAGTAYDLTAVEAVARTIASIDSGPKIIVEKSTVPVTTSDRILQVFSACGVQERLEVLSNPEFLAEGQAIENLEHPDRVLIGSRGTESGIKAEKKLAALYQHWISDDRIITTNIWSAELAKLAANAFLAQRISSINAISAICEKTGADVNEVAHVIGTDTRLGSKFLKAGVGFGGSCFRKDLLGLVYLCKSFQLEEVADYWVQVLKLNDFQKERYVRLVLESMFGTICRKKIAVLGFAFKSNTGDIRDSPAITIVERMVNEKADVHIYDPKVRPENIHNLFPDVTCHKDPYEATENAHAILVLTEWEEFSLLDYKRIYGSMQRPAFLFDGRAYLDHVYLKNLGFQVQSIGKGKL

SEQ ID NO: 111 - DNA, 1362 bp, Paramoeba atlantica
  source 1..1362: mol_type = genomic DNA
atggatgtggaaacaaaagaagttgtttgtattggggcgggatatgttggtggacctaccatgactgttatggccaaacattgtcctgagattaattttcatgttgttgatattaatgaaaagagaatcaatgagtggaactctgagattcttcccatttatgaacctggtctcgatgaaatggtcaaacaagtgagaggaagaaaccttttctttagcacaaatgtaaaagaggcaattgcaaagggagatattatttttgtctctgtcaatacatctactaaagaatatggccttgatgctggaacagcgtacgatctcacagctgtggaagcagtggccagaaccatcgcctctatcgattcgggaccaaaaattattgttgaaaaatccaccgtccctgtgaccacttctgatcgaattcttcaagtttttagtgcttgtggtgttcaagaaagacttgaggttttatcaaacccagagtttttggctgagggacaagcaattgaaaatttggaacatcctgatcgtgttcttattggttcccgtggaactgaatccgggattaaagctgaaaagaaattggctgcgttgtatcagcactggatttctgatgatagaattattaccacaaatatttggtctgctgagttggctaaacttgctgctaatgcttttttggctcagagaattagttcaattaatgctatttctgctatttgtgaaaaaacaggagcagatgttaatgaagtggctcatgtcattggtaccgacactcgattagggtcaaaatttttgaaggctggagttgggtttggagggtcttgtttccgaaaggatcttttgggtttggtctatctttgcaaatcttttcaacttgaagaggtcgcagattattgggtccaagttttgaaattgaatgatttccaaaaagaaagatatgttcgtcttgttctggaatctatgtttggaacaatttgtagaaaaaagattgcggttcttggttttgcttttaaatctaacactggtgatattcgtgactctcctgctattactattgtggagcgaatggtgaatgagaaagctgatgttcatatttatgaccctaaggtgagacctgaaaatattcataacttgtttcctgatgtgacatgccacaaagatccttatgaagccacagaaaatgctcatgctattcttgttcttactgaatgggaagagttttctcttttagattacaagagaatctatggctcaatgcaacgtcccgcttttctttttgacggacgagcttatttggatcacgtgtatttgaagaatcttggattccaagttcagtcaattggaaaaggaaaactgtaa

SEQ ID NO: 112 - DNA, 1306 bp, synthetic construct
  source 1..1306: mol_type = other DNA
aaccatgaccgtcatggcaaagcactgtcccgaaatcaattttcatgtggttgacataaatgagaagaggattaacgagtggaatagtgagatcttaccaatttacgagcctggattggatgagatggtaaaacaagtaagaggtcgtaatctatttttctccaccaacgtaaaggaagctattgctaaaggcgacattatctttgtgtcagtaaacacatctacaaaggaatatgggttagatgcaggcacagcttacgatctaaccgccgttgaggcggtggcaaggaccattgcctctatagattcaggccccaagatcatcgtcgagaagagtacggtcccggtaaccacgtcagataggatactacaagttttcagcgcatgcggcgtgcaggaacgtcttgaagtgctgagcaacccggagtttcttgcggaggggcaggctatcgaaaaccttgagcacccagacagagtccttataggaagcaggggtacggaatctggcataaaggccgagaagaaactggcagcgttataccaacattggattagtgacgatcgtattataacgacgaacatttggagtgcggagctagcgaaacttgcggcgaacgcattcctggcacagaggatcagtagtataaatgctatcagcgctatctgtgagaaaaccggggctgacgtgaatgaagtggcacacgtcataggcacagatactagattaggatccaagtttttgaaggcgggcgtagggttcggaggttcttgcttcagaaaagatttgttgggtctagtgtatttgtgtaaaagtttccagcttgaagaagtggctgattattgggttcaggttctgaaattaaacgattttcaaaaggaaagatacgtgagattagtgttggagagcatgttcggtactatttgcaggaagaaaatcgcggtgttaggttttgcgtttaaatccaacaccggtgacatcagggactctccagctataactatcgtggagcgtatggttaacgaaaaagccgacgttcacatctatgaccccaaggttagacccgagaatattcataatttgttcccagacgtcacctgtcataaggatccttatgaagcgactgaaaatgctcacgcgatcttggttttgacagaatgggaagagtttagtcttctggattacaaaagaatatatggtagcatgcagagacctgcattcttatttgacggacgtgcttatttagatcatgtttatttgaagaatctggggtttcaggtccagtctatcggtaaggggaagttgtga

SEQ ID NO: 113 - protein, 398 aa, Quillaja saponaria
MPTLYKKAGLMASASRVDLDGNQIKPMTICMIGAGGFIGSHLCEKIMAETPHKVLALDVYNDKIKHLLEPDSLQWKDRIQFHRINIKHDSRLEGLIKMADLTINLAAICTPADYNTRPLDTIYSNFIDALPVVKYCSENNKRLIHFSTCEVYGKTIGSFLPKDSPLLKDPEYFVLKEDASPCIFGPIEKQRWSYACAKQLIERLVYAEGAENGLEFTIVRPFNWIGPRMDFIPGIDGPSEGVPRVLACFSNNLLRGEPLKLVDGGQSQRTFVYIKDAIEAVLLMIENPARANGHIFNVGNPHNEVTVRQLAEMMTEVYSKVSGEPSLEVPTIDVSSKEFYGEGYDDSDKRIPDMTIINRQLGWNPKTSLWDLLESTLTYQHRTYAEAIKKSIAKPVAS

SEQ ID NO: 114 - DNA, 1197 bp, Quillaja saponaria
  source 1..1197: mol_type = genomic DNA
atgccaactttgtacaaaaaagcaggcttaatggcgtcggcgtcaagggtagatctggatggtaatcagataaagccgatgacaatttgcatgatcggtgccggtgggttcattgggtcccacctctgcgagaagattatggcggagacaccgcacaaggttctggcattagatgtctacaatgacaagatcaagcacttactggagccggattctcttcaatggaaagatcgcatccaattccaccgcatcaacattaagcacgattcgaggctcgaaggtctcatcaagatggcagatctgactataaatctggctgctatttgtactcccgcggattacaacacccgtcctctggacacaatttatagcaatttcattgacgctcttcctgtggtaaagtactgttcggagaataacaagcgtctcattcatttctctacgtgtgaagtgtatgggaaaacgattgggagctttctcccaaaagacagccctcttctaaaggatcctgaatattttgttcttaaagaagatgcctccccatgcatatttggtcctattgaaaagcagagatggtcctacgcatgtgcaaagcaattgattgagaggctggtttatgctgagggtgctgagaatggccttgagttcactattgtgcgacctttcaattggattggccccagaatggatttcatacctggcattgatggtccaagtgaaggtgttccacgggttctggcatgctttagtaacaatctccttcgcggtgagccactcaaactcgttgatggtggccaatcccagagaacttttgtttatatcaaggatgcaattgaagctgttttgttgatgattgaaaaccctgccagggctaatggtcatatttttaatgtgggtaaccctcacaatgaagttacagttcggcaacttgctgaaatgatgaccgaggtctattctaaggtaagtggagaaccgtctcttgaggtgcctaccattgatgtaagctccaaagaattttatggtgaaggatacgatgatagtgacaagagaattcctgacatgaccataatcaacaggcaacttggctggaaccctaagacatcgctctgggatcttcttgagtcgaccctcacctatcaacataggacatatgcagaagctattaagaaatcaattgcgaaaccagttgccagctag

SEQ ID NO: 115 - DNA, 1197 bp, synthetic construct
  source 1..1197: mol_type = other DNA
atgccgactctttacaaaaaagcaggattaatggcgagcgcatcaagagtggacttagatggcaatcaaattaaaccaatgacgatttgcatgattggggctggaggctttataggaagtcacctgtgtgaaaagataatggctgaaacacctcataaggtattagcccttgatgtctacaacgataaaatcaaacatttgctggagcccgactctctacagtggaaggatagaattcagttccatcgtatcaatatcaaacatgacagtcgtttggaagggctgattaagatggcggacctaaccattaatttagcagccatttgtacacccgccgattacaacacaaggcccctagatacaatctactctaacttcatcgacgccctaccagtagtcaaatattgttcagagaataacaaacgtttaatacattttagcacctgtgaagtatatgggaaaacaatcggcagctttctacctaaggactcccctctgttaaaggatccagaatattttgtattaaaagaggacgctagcccgtgtatatttgggccaatcgagaaacaacgttggtcttacgcctgtgcaaagcaattaatcgaaagactggtctatgcggaaggtgcggaaaacgggctagagtttacaatagtcagaccatttaattggatagggccgagaatggactttatccctgggattgacggtcccagcgagggtgtaccgagagttctggcatgctttagtaacaacttactgagaggagagccactaaagctagtggatggtgggcagtcccaaaggacgttcgtatatatcaaagacgccattgaagccgtcttacttatgatcgaaaaccccgcccgtgccaacggtcatatttttaatgtaggaaaccctcataatgaagtgacagtaaggcagcttgcggagatgatgactgaagtttattcaaaggttagcggagagcctagcttagaagtaccaacaatcgacgtctctagcaaagaattttacggcgaaggttatgacgacagcgataaaagaatcccggatatgaccataattaacagacagctgggatggaatccaaagacgagtctgtgggacctgctggagagcacgctgacataccaacatagaacgtacgctgaggccattaagaagagcatagctaagcccgttgcctcctga

SEQ ID NO: 116 - protein, 492 aa, Quillaja saponaria
MVESPADHDVLKIIVLPWVTSGHMIPMVDAARLFAMHGADVTIITTPANALTFQKSVDRDFNSGRLIRTHTLKFPAAEVGVPEGVENFNNTSPEMTSKVYLGVSMLREPTQQLIEDLRPDCLITDMFYPWAVDVADKLGIPRLIFQGPGSFGLSAMHSIKQYEPFKSVTSDTETFPLPGLPHKVEMTRLQIPKWVREPNGYTQLMGRVKDSERRSYGSLVNSFYDFEGPYEEHYRKATGQRVWSIGPVSVWVNQDAADKVGRGQDLVAEDQNSWLNWLNSKEKNSVLYVSFGSMAKFPSAQLLEIAHGLEASGHSFIWVVRKVDGDDDVDVWLPDFEKKMKENNKGFIIRNWAPQLLILDHPAIGGLLNHSGWNSVLEGATAGLPMITWPLYAEHFYNERLVLDVLKIGVPVGVKEWKNLHEVGELVRRDAIAKAIKLLMGSGEEAEVMRKKAKELGVGAKKGIQVGGSSHTNLIAVIDELKSLKKSRIQGV

SEQ ID NO: 117 - DNA, 1479 bp, Quillaja saponaria
  source 1..1479: mol_type = genomic DNA
atggtggagtctccagcagatcatgatgtgctcaaaatcattgtccttccatgggtaacctcaggtcacatgattcccatggtagatgcagccagactatttgctatgcatggtgcagatgttaccatcatcaccaccccagctaatgcccttacattccagaaatccgtcgaccgtgatttcaattccggtcgtttaatcagaactcacacccttaaattccctgcagcagaagttggtgtacctgaaggagttgaaaacttcaacaatacttcccctgaaatgacctccaaagtctaccttggagtctcaatgctccgagaaccaacccaacaattgattgaggatctgcgtccagattgtcttatcactgatatgttctatccttgggctgtggatgttgctgacaaattaggcattccaaggctaatttttcaaggtcctggaagttttggtttgtcagctatgcattctatcaaacagtatgagccctttaagtcagtaacttcagatactgagacattcccactacctggattgccgcataaggtagagatgacaaggttgcagataccaaaatgggttcgtgagccaaatgggtacactcaattgatgggcagggtaaaagattcggagagaagaagctatgggtcattggtgaatagcttttatgacttcgaaggcccttatgaagagcactataggaaggcaacaggacagagggtttggagcattggaccagtttcagtttgggtgaaccaagatgctgcagataaggttggaagaggacaggatcttgttgctgaagaccaaaacagctggttgaattggctcaattccaaagagaaaaactctgttctgtatgtaagttttgggagcatggccaagttcccatctgctcagcttcttgaaatagctcatgggcttgaagcttcaggtcatagtttcatctgggttgtcagaaaagttgacggggatgatgatgtagacgtgtggcttccagattttgagaagaaaatgaaagagaacaacaagggtttcatcataaggaattgggcaccacaattgctcatattggaccatccagcaattggaggtttgctgaatcacagtggatggaattcagtactggaaggtgctacagcaggcttgccaatgatcacttggcctctgtatgccgagcatttttacaatgaaaggttggttctagatgtgttgaaaattggagtaccagttggggtgaaggagtggaagaacttgcatgaggtgggtgagttggtgagaagggatgcaattgccaaggcaattaaattgttaatgggtagtggagaagaagctgaggtaatgaggaaaaaagccaaagagcttggtgttggagcaaagaaaggtattcaggttggaggttcttctcataccaatttgatagcagtgattgatgagttaaagtcactaaagaaatcaagaattcagggtgtctga

SEQ ID NO: 118 - DNA, 1480 bp, synthetic construct
  source 1..1480: mol_type = other DNA
gatggtcgagtcaccggcagatcatgacgttctaaagattattgttctaccttgggtaacgagtgggcatatgatccccatggtcgacgcggctaggcttttcgcaatgcatggcgcagatgtaacaattataactactccagcgaatgcgcttactttccaaaagagtgttgacagggattttaacagtggtcgtttaatcagaacccacaccctgaaatttccagcagccgaggtaggcgtacctgaaggcgtcgaaaactttaataacacatcaccagaaatgacgagtaaagtatacttaggggtttccatgcttagagaaccaacccaacaattgatcgaagaccttagacccgactgtctaattacggatatgttctacccatgggccgtcgatgtggctgacaaactaggtataccgaggttaatattccagggtccagggtcttttggacttagtgcaatgcactcaataaaacagtatgaaccgttcaagtcagtcaccagtgacactgaaactttcccgttgcccggattacctcacaaggttgagatgaccaggctacaaatcccgaaatgggtaagggaacctaacgggtacacccaattgatgggtagggtaaaggacagtgaacgtagaagctatggatctctggtaaatagcttctatgacttcgagggtccttatgaggagcactacaggaaggcaacgggccagcgtgtttggagcataggcccagtttctgtatgggttaatcaagacgcggcggataaagtaggaagaggtcaagacttggtcgctgaagatcagaacagttggcttaactggctgaactctaaggagaaaaactctgtcttatacgtgagcttcggaagtatggcaaagttccctagcgcccaactgcttgagatcgcccacggtctagaggccagtggacattcttttatttgggtcgtacgtaaggtcgacggagatgacgacgtggatgtttggttgccagatttcgaaaaaaagatgaaggagaataataaaggtttcataatcagaaattgggctcctcagttactaattttagatcacccggcaattggcgggcttttaaatcattcagggtggaacagtgtgcttgaaggagcaaccgcagggcttcccatgatcacctggcccttgtatgctgaacacttttacaacgaaagattagtactagatgtgcttaagattggcgtgcccgtcggcgtgaaggaatggaagaacttgcatgaagtgggagaattagtaaggagggacgcaatcgcgaaagctatcaagcttctgatgggtagtggggaggaagccgaagttatgagaaaaaaggcaaaagagttgggggttggggccaagaaaggcatccaggtgggcggaagctctcatactaacctaatcgccgttattgatgagctgaagagcttgaagaaatccaggatacagggtgtataa

SEQ ID NO: 119 - protein, 494 aa, Quillaja saponaria
MVSGDDDVSRRPLKVYFIAHPSPGHIAPLTKIAQLFAARGEHVTILTTPANVHFHEKSIDKGKTSGYHVNIHAVKFPSKEVGLPDGIENFSHASDNETAAKIWAGFSMLQTEMEQYMEQNPPDCIVADMFNRWTSDFAIKLGIPRIVFNVYCIFTRCLEEAIRSPDSPHLKLNSDNEQFIIPGLPDPITITRAQLPDGAFSVVKEQVSEAELKSFGMVINGFSELETEYIEYYKNIMGRKRIWHVGPLQLIYQNDDPKVQRSQKTAVVSDNELVSWLDSKKPDSVIYISFGSAIRFSNKQLYEIACGLEASGYPFLWALLWVPEDDDDVGNKWLPDFEERIKRENKGIIFRGWAPQMLILNHPAIGGFMTHCGWNAVVEALSFGVPTITLPVFSEQFYTERLISQVLKTGVEVGAEKWTYAFDAGKYPVSREKIATAVKKILDCGEEAEGMRKRAREMKEKAQKSVEEGGSSYNNLTAMIEDLKEFRANNGKVA

SEQ ID NO: 120 - DNA, 1485 bp, Quillaja saponaria
  source 1..1485: mol_type = genomic DNA
atggtctccggcgacgacgacgtttctcgtcggccactgaaagtttactttattgcacacccctcacctggccatattgcccctctaaccaaaatagcccaactctttgctgcacgtggtgagcacgtgactattcttactactcccgccaatgtccacttccatgagaaatccatcgacaaaggaaagacttccggctatcatgttaacatccacgccgttaaatttccttctaaagaggtcggtctccccgacggcatcgaaaacttctctcacgcctccgataatgaaacagcagccaaaatttgggccggattctccatgcttcaaactgaaatggagcaatatatggaacaaaacccacccgattgcattgttgccgacatgttcaaccgctggacttccgacttcgctatcaaattgggaatcccgagaatagttttcaacgtctactgtattttcacacgctgtttggaagaagcaatcagatcacctgactcgccacacttgaaactaaactccgataatgaacagtttattattccgggtctacccgaccccataacaattacccgagctcaactgcccgacggtgccttttctgtcgtcaaagaacaagttagtgaagctgagttgaaaagcttcggaatggtgatcaacgggttttccgaactcgaaaccgaatacatcgagtattacaagaatatcatgggtcgaaaacggatttggcatgtcggaccccttcagctcatttaccaaaacgatgaccccaaagttcagaggagccagaagacagcggtcgtgagtgacaacgagttagtgagttggcttgactcgaagaaacccgactcagtgatttacatttccttcggtagtgcaattcgtttctctaataagcagctctatgaaatagcatgtggattagaagcttccggctacccatttttgtgggccttactttgggtgccagaagatgacgacgacgtgggcaacaaatggttgcctgatttcgaagaaagaataaaaagagaaaataagggaataattttcagggggtgggccccacagatgttaatcttaaaccacccggcgatcggtggtttcatgacgcattgtggttggaatgcggtggtggaagcgctttctttcggtgttccgactattacgcttccggttttctcggagcagttttatactgagagactgatatcacaagtgctcaagactggtgtcgaggtcggtgcagagaagtggacctatgcatttgatgcggggaaatatccggtgagtcgggaaaagatagcgacggcggtgaagaagatattagactgtggagaagaggcagaaggaatgagaaagcgggccagggagatgaaagaaaaagcccaaaaaagtgttgaagaaggtgggtcctcttataataatttaacggctatgattgaagatcttaaagaatttagggctaacaatggcaaggttgcatga

SEQ ID NO: 121 - DNA, 1422 bp, synthetic construct
  source 1..1422: mol_type = other DNA
atggcgaaaacagataagcaactgcacattgctatgttcccttggttagctatgggccacatctttccgaattttgagcttgcaaagctgtttgctcaaaaaggacattcaatcactttgatttctacgccccgtaatatcagtagacttcctcaaatccccacccacttggaacaattaatcaaactggtaagcttgcccatactgcctaagcataaggccaacctgcctgaaaatgcggaatctactatggacgttactccgaacaaagtcccatatctgaaaatggcttatgacgggctacaagagagccttacgcaactattgaagagtagcgctcctgattggattctgtatgacttcgcggctgattggctgccaccgttagtccactctttacaaatacgttgtgtttttttcgtagtaagtccggcgtggaatttgtgcttttttgatactccaaagccccaattaggtagtgcggccgttttccgtaccaagcccgaggattacctgaggcctccatcatgggttccctttcacagcaatatcggacttaaattgcacgaggtgaagaaaatgttcgagggagtctctgataaggaaactggagtaactgtgtccttcaatttcaacaaggccgtgagctcctgtgatttgttctccttccgttcctgctacgagcttgaaagcgagtggctaaacttagtagaggatatttacaaaagaccagttgtgcctgtaggcgtaatacctccaagcttccaagtaagaatcgttaatgaagaggacaacaaacccgaatggctaaaaatccaatcttggttggataagcaggagcaaggatctgttgtgtacatagcattcggcagcgaattgaaactgggccagcaggacttgacagaacttgctctaggcttggaactatccgggctgccttttttctgggctcttcgtaagcaacaagattctagtagtgtagacttacccgacggctttgaggatagagtaagcgacagaggagtagtgtgtcgtgattgggtgcctcagctaaagatccttgcccatgggagtatagggggttatctgacgcactgtggctccggttcagtcattgaaggcctacactttgggagagtactagtgatgctaccatacctgctagatcaagccctgtatgcgagagttctggaagaaaagaaacttggcgttgaaattcccaggaatgagcaggacggaagctttacacgttcaagtgtcgctaaatcagtgaagctagcgatagtcgatgagggaggctccatttacagagataaagcgaaggaaatgggtctagtattttcagacaaggacaggcatgaacagtatattgagaatttcttacaacacctgcagcataaaagagagccttttcaaatataa

SEQ ID NO: 122 - protein, 504 aa, Quillaja saponaria
MVSGDDDVSRRPLKVYFIAHPSPGHIAPLTKIAHLFAALGEHVTILTTPANVHFHEKSIDKGKASGYHVNIHTVKFPSKEVGLPDGIENFSYASDVETAAKIWAGFAMLQTEMEQYMELNPPDCIVADMFTSWTSDFAIKLGITRIVFNVYCIFTRCLEEAIRSPDSPHLNKEISDNEPFVIPGLPDPITITRAQLPDGTFSPMKELARTAELKSFGMVINGFSELETDYIEHYKKIMGHKRIWHVGPLQLIHRNDEDKIQRSHKTAVLSDNDNELVSWLNSKKPDSVIYICFGSATRFSNHQLYEIACGLEASGHPFLWGLLWVPEDEDNDDVGNKWLPAFEERIKKENKGMILRGWAPQMLILNHPAIGGFMTHCGWNAAVEALSSGVPIITFPVFSDQFYNERLISQVHKCGVGVGTEAWSYAFDAGKNPVGREKIMTAVKKILDGGEEAEGMRKRARELKEIAKRSVEEGGSSYNNLTAMIQDLKEFRANNGKAAQDHES

SEQ ID NO: 123 - DNA, 1515 bp, Quillaja saponaria
  source 1..1515: mol_type = genomic DNA
atggtctccggcgacgacgatgtttctcgtcggccactgaaagtttacttcattgcacacccctcacctggccatattgcccctctgaccaaaatagcccatctcttcgctgccctcggtgagcacgtgactattctcactactcccgccaatgtccacttccatgagaaatccatcgacaaaggaaaggcttccggctatcatgttaacatccacaccgttaaatttccttctaaagaggtcggtctccctgacggcatcgaaaacttctcttacgcctccgatgttgaaacagcagctaaaatttgggctggattcgccatgctacaaactgaaatggagcaatatatggagcttaacccacccgattgcatcgttgccgacatgttcacctcctggacctccgactttgctatcaaattgggaatcacaagaatcgttttcaacgtctattgtattttcacacgctgtttggaagaagccatccgatcaccggactcgccacacttgaacaaagaaatctctgataatgaaccgtttgttatcccgggtctaccagaccccataacaattacccgagctcaactgcccgacggtaccttttctcccatgaaagaactagctagaacagctgagttgaagagctttggaatggtgatcaacgggttttccgaactcgaaaccgattacatcgagcattacaagaaaatcatgggtcacaaacggatttggcatgtcggaccccttcagctaatccaccgtaacgatgaagacaaaattcagaggagccacaagacagcggtgctgagtgataacgataacgagttagtgagttggcttaactcgaagaaacccgactcagttatttacatttgcttcggtagtgcaactcgtttctctaatcaccagctctatgaaatcgcctgtggattagaagcttccgggcacccatttttgtggggcctactttgggtgccagaagatgaagataacgatgacgtgggcaacaaatggttgccagctttcgaagaaagaattaaaaaggaaaataagggaatgattttaagggggtgggctccacagatgttaatcttgaatcacccggcgatcggtggtttcatgacgcattgtggttggaatgcggcggtggaggcgctttcttccggtgttccgattattacatttccggttttctcggatcagttttataatgaaaggctgatatcacaagtgcataagtgtggggtgggggttggtacggaggcgtggagctatgcattcgatgccgggaagaatccggtgggtcgggaaaagataatgacggcggtgaagaagatattagacggtggagaagaggcggaaggaatgagaaagagggcccgggagctgaaagaaatagctaaaagaagtgtggaagaaggtgggtcctcttataataatttaacggctatgattcaagatctgaaagaatttagagctaacaatggcaaggctgcacaagatcatgaatcgtga

SEQ ID NO: 124 - DNA, 1515 bp, synthetic construct
  source 1..1515: mol_type = other DNA
atggtctccggcgacgacgatgtttctaggagaccgcttaaagtctacttcatcgctcatccatctcccgggcacatcgcaccgttaacgaagatagcgcacttatttgccgcacttggcgagcatgtcactatactgacgacgcccgcaaatgtccacttccatgagaaatcaatagacaagggaaaggctagcggctaccacgtgaatattcacactgtcaaattcccgtccaaagaggtgggattacccgacggtatcgagaacttcagttatgcgagtgatgttgaaactgctgcgaaaatttgggcaggatttgcaatgctacaaacggaaatggaacaatatatggaattgaaccccccagattgtatagtggcggatatgtttacctcctggacaagcgactttgcgataaagcttgggataactaggatagtctttaacgtttactgcatttttacaagatgtcttgaggaagccatccgttctccggatagccctcacttaaacaaagagatatctgacaacgagccgttcgtaatacccggtttacccgaccccatcacgatcacgcgtgctcaacttccggatggaactttcagcccgatgaaggaattggcaaggaccgcagaactgaaaagttttggaatggtgattaacggttttagtgagcttgagaccgactatatcgaacattacaaaaagataatgggccacaagagaatttggcatgtcggtccgttacagctaattcacaggaatgacgaagataagatccagaggagccataagactgcggttctgtctgacaatgataacgaactggtgtcttggttgaacagtaaaaagccggacagtgtgatatatatttgttttgggtctgcgacaagattttcaaaccaccagttatatgaaatcgcctgcggactggaagctagtggtcatccttttctatggggcctgctttgggttcccgaggacgaggacaacgacgacgtgggaaacaagtggcttcctgcttttgaagaaaggatcaaaaaggagaacaagggtatgatacttaggggttgggctccccagatgctgatactgaatcacccagcaattggaggatttatgactcattgcggctggaatgccgctgtggaggcgttatcttcaggagtgcccattatcactttcccggttttctcagatcaattctataatgaaagactgatatcacaagtacacaaatgtggggtaggagtgggcaccgaagcatggtcctacgctttcgatgcaggaaaaaatcctgttggtagggagaaaataatgacagccgtgaagaaaatcttagacggtggtgaagaagctgaggggatgagaaagagggcaagggagttgaaagaaatagctaagcgttcagtagaggaagggggttcatcctacaataacctgacggcgatgatccaggacctaaaggagtttagggccaacaatggaaaggcagcacaggaccacgagtcataa

SEQ ID NO: 125 - protein, 464 aa, Quillaja saponaria
MAAAAPNHRLHIAFFPWLAFGHINPFFELAKLIAQKGHHISFISTPRNIQRLSQVPPQLADSIDLVSLPVIHNSNLPENAESTMDIPPDKTPYLGMLHDSLKEPLTQFLQTHSPDWILYDFSAGWLAAIVEDLGISHGYFSIIPCWNIGFNGRQMNGFQKPDISLPSAVSLKKYEVKKIMDLVKSFPKILDESATKSIASHSTCEVIFIRNCPEIEADWFDYTSKIFDKPVVPVGVVPPSVHITNKEKDEHFNKWLEIKEWLDQQDRGSVIYIAFGTESLPNQDEITMLAQGLELCGLPFFWALRKSNVASDQPNSDSVELPEGFEEQTKGRGIVWTSWAPQQRILGHNSIGGFVTHCGWSSVIEGIHYGRPLIMFPLTVEQSLNARILGEKKLGMEVPREDDGSFTGEVVAETLKLVLLDQDGKVYRDKVTEMSKVFGDKDKHEKYMGDLLEFFKNYRSLKRN

SEQ ID NO: 126 - DNA, 1395 bp, Quillaja saponaria
  source 1..1395: mol_type = genomic DNA
atggctgctgcagctcccaatcacaggctccacatagcattcttcccatggttagcatttggtcacataaaccctttctttgagcttgcgaagctcattgctcaaaagggtcatcatatttctttcatttccaccccaagaaacatccaacgcctttcacaagttcctccacaattagcagattccatagatctagtgagcttaccagtaatccataattcaaacctcccagaaaacgcagagtccaccatggacattccacctgataaaaccccttaccttgggatgcttcacgacagtctcaaagaaccccttactcaattccttcaaactcattcccctgattggattctgtatgacttttcagctggttggcttgctgccatagtagaagaccttggtatctcccacggctacttttctatcattccctgttggaacataggcttcaatggacgccaaatgaatggtttccaaaagccagatatttcattgccctccgccgtgtcgcttaagaaatatgaggtgaagaaaatcatggatttggtcaaatcttttcccaaaattttggatgagtcggccactaaatccatagcttcgcattcgacctgtgaagtaatttttatacgaaattgccccgagattgaagcagattggttcgactatacttcgaaaattttcgataaaccagtggttccggtgggcgtagtgcccccatctgtgcacataactaacaaagagaaggacgagcatttcaacaaatggttggagatcaaagaatggttggatcaacaagacagaggttctgtaatttatatagcttttggaactgaatcactgccaaatcaggatgaaatcaccatgcttgctcaagggcttgagctatgtgggcttcctttcttttgggcattaaggaagtctaatgtggcttctgatcagccaaattcagactcagttgagctaccggagggatttgaagaacaaaccaaaggtcgtggaattgtgtggacgagttgggcacctcaacagagaattctgggtcacaattcaattgggggttttgtgactcactgtggttggagttcagtaatagaaggaattcactatggacggccactgattatgttccctctaacagtcgaacagtctctgaatgctaggattttgggggagaagaagttgggtatggaagtacccagagaagatgatgggtcttttacaggtgaagttgtggcagagacattgaagctggtattgctggaccaagatgggaaagtttacagggacaaggtaacagagatgagtaaggtatttggggacaaagacaaacatgagaaatacatgggtgatctacttgaattcttcaaaaattacaggtctcttaagaggaattag

SEQ ID NO: 127 - DNA, 1395 bp, synthetic construct
  source 1..1395: mol_type = other DNA
atggcagcagcggcacccaatcatcgtctgcacatagcgtttttcccctggctagctttcggccacatcaatccatttttcgaactagcaaagttaatcgcgcaaaaaggtcatcacatcagtttcatctccactcccagaaatatccagaggttaagtcaggttccgcctcagctagctgactccatcgatctggtatccttgcccgtaatacataactcaaatcttcccgaaaatgccgagtcaacaatggacatcccgccagacaaaaccccttaccttgggatgttgcacgactccttgaaagagccactaactcaatttcttcaaacacactccccggattggattttatatgatttctcagctggatggttggcagctatagtggaggatcttgggatcagtcacggttatttctcaataataccctgttggaacatcggctttaatgggagacagatgaacggttttcaaaaacccgatatcagtctaccttcagccgtttcactgaagaaatacgaggtcaagaaaattatggatctagtaaaaagtttcccgaaaattctggatgaaagcgccacgaaaagtatcgctagccactcaacctgtgaagtcatatttatcaggaattgccctgaaattgaagccgactggttcgattatacatcaaagatattcgacaagcccgtagtgcctgttggcgtggtgcctccgtcagtccatattaccaacaaggaaaaggatgagcattttaacaaatggttagaaataaaagagtggctggaccaacaggataggggtagtgtcatatatatcgcgtttggtactgagtccttacccaatcaagatgaaataactatgctagctcagggactggaactttgtggcctgccgtttttctgggcgttgaggaagtcaaacgttgcctcagatcagccgaactctgacagtgtggaattgccggagggcttcgaggaacaaactaagggaagagggattgtctggacctcatgggcaccacaacagagaatcctaggccataatagtatcggtgggtttgttactcattgtggctggtccagcgtaattgaagggatacactacggtaggcctttgataatgttcccgctaaccgttgaacaatctctaaatgctaggatactgggagagaaaaagttggggatggaggtaccgcgtgaggatgacggttcatttactggagaagtagttgccgagacattgaaacttgttttgttggaccaagacggaaaggtttaccgtgacaaggtaacagagatgtcaaaagtgttcggtgacaaagataaacatgaaaagtacatgggggacctactggagttctttaagaattataggtctctgaaacgtaactaa

SEQ ID NO: 128 - protein, 509 aa, Quillaja saponaria
MDSTHLQPATTPLKIHFIPFISPGHIIPLSELARIFASRGEHVTIITTPFNADLLQKSIDEDRDSGEHIGIHTVEWSATELGLPHGIENLSNVTDLETAIKLHRALMLMQKQMEDFMTRNPPDCIIADTFYPWASEFANRMGIPRLIFYPWSTFALCLMESIRSPDSPHRRLSSDSDPFVVPGLPHPIILTRSQLPEHDRKDIADPAAQLMDQHKETEMKSYGIILNNFAEIETEYTEHYKKITGHKVWHIGPAAAIVHRNAKEKAERVFKSDEHDNNLVINWLNSKEPNSVVYVCFGSGCQFPDKQLYEIACGLELSGHQFVWVVRGKDKQIDVNDDGEKTWLPKGFEERMKTENKGLIVRGWAPQVLVLDHPSLGCFLTHCGWNSTIEGITAGVPLITWPVFAEQFYNEKLITQVHGNGVVVGSEEWIMLFTVAKSLVSRDKIENAVRKIMDGGDEAVQIRRRARELGEKAWKAASTGGSSYNNLTAAIEDLKRLREDRSKLKTKTI

SEQ ID NO: 129 - DNA, 1530 bp, Quillaja saponaria
  source 1..1530: mol_type = genomic DNA
atggactccacccacttgcagccggccactactcctctgaaaattcacttcatacctttcatatctccgggtcacatcatcccactctctgaattggctcgtatctttgcttcacgcggtgagcatgtgactatcattaccactcctttcaacgctgacctacttcagaaatccattgacgaagacagagattccggcgagcacattggcatccacaccgttgaatggtcagccacagagctaggccttcctcatgggattgaaaatctcagcaacgtcactgacttggagaccgccattaagctacacagggccctcatgctaatgcagaaacagatggaagatttcatgacccgaaatccacccgattgtataattgccgacacgttttacccgtgggcctccgaatttgctaatcggatgggtatcccgagactcattttctacccgtggagtactttcgcgctctgtttgatggaatctattcgatctcccgactccccgcaccgaagattgagttcggattcggatccatttgtggttccgggtcttcctcacccgatcatcttgacccgttctcagcttccggaacacgatcgaaaggacatagcggacccggctgcccaactcatggatcagcataaagaaactgagatgaagagctacggaattattctcaacaatttcgcggagatcgaaacagaatacacagagcattacaagaaaataacgggtcacaaggtttggcacattggacctgccgcagcaattgttcaccgaaatgccaaagagaaggcagagagggtattcaagagtgatgagcatgacaataaccttgtcatcaattggctcaactcgaaggaaccaaactcagttgtttatgtttgtttcggcagcggatgtcaattccctgataaacaactctatgagattgcatgcgggttagagttatctgggcatcaatttgtttgggtggttcgcggaaaagataaacaaatcgatgttaatgacgatggggagaagacatggttgcctaaagggtttgaggaaagaatgaaaacagaaaataaaggtttgattgtaaggggatgggccccacaggtgctggttttggatcatccatcattgggatgtttcttgacgcattgcggctggaattctacgattgagggaatcacagcaggcgttcctttgatcacgtggccagtattcgccgagcaattctataatgagaagctaatcacgcaggtgcatgggaatggggtggtggttggttcagaggagtggatcatgttgttcaccgtcgctaaaagcttggtaagtagagacaaaattgagaatgctgtgaggaagataatggacggtggtgatgaggctgtacaaatcagaaggcgggcccgggaacttggagaaaaagcttggaaagctgcttcaactggggggtcctcctacaataatctaaccgcagcaattgaagaccttaagcggttgagagaggaccgttcgaagctgaaaacaaaaacaatttga

SEQ ID NO: 130 - DNA, 1530 bp, synthetic construct
  source 1..1530: mol_type = other DNA
atggatagtacacaccttcaaccggcgaccacaccattgaagatacatttcatacccttcatttcacctggccacatcatccccctgagtgaattggcgcgtatattcgcgtccagaggcgagcacgtgacgataataacgacgccctttaacgcagatttgcttcaaaagtctatcgacgaggatagagattcaggtgaacatatcggcatacatactgtggagtggtccgcaactgaactgggtttgcctcatgggatcgagaatctaagcaacgtaaccgacctagagaccgccattaaacttcaccgtgcgctaatgttgatgcaaaagcaaatggaggacttcatgacgagaaatcctcctgactgtatcatagcagacactttttacccgtgggcgagtgaatttgcaaataggatgggcataccacgtcttatcttctacccgtggagcacatttgcgctgtgcctaatggaatcaatacgtagtcctgactccccacataggcgtctgtcatccgacagtgacccgttcgttgtgcctgggttaccacaccccataatccttaccaggtctcaactaccggagcacgacagaaaagacatcgctgatcctgccgcgcaattaatggaccagcataaagagaccgagatgaagtcttatggtataatcttaaacaattttgcggagattgaaacagagtataccgagcattacaaaaagattacggggcacaaggtttggcacataggtcctgctgctgctatagtccatcgtaacgccaaggagaaagcggagagggtttttaaaagcgacgagcatgacaacaatcttgttataaactggctaaatagcaaggaacctaactccgttgtctatgtctgcttcggctccgggtgtcaattccctgataaacaactttacgagattgcttgcggtctagagttgtccggtcatcagtttgtgtgggtggttaggggaaaagacaagcaaatcgacgtgaatgacgacggtgaaaagacttggctgccgaaaggatttgaggagaggatgaaaactgaaaacaaaggactaattgttcgtggatgggccccccaagttttagttctggatcatccaagccttggttgtttcctaacccattgcggctggaacagcacgatagaaggtataacagcgggggtaccgctgattacttggccagttttcgccgaacaattttataatgagaaacttattacccaggttcacggaaatggcgtagtcgttggttcagaggaatggattatgttgttcacagtcgcaaaatctctggtatccagggacaaaatagagaacgcagtcagaaaaatcatggacggaggcgacgaagcagtccagataaggcgtagggccagagaactaggtgagaaagcgtggaaggcagcttctaccggaggctcatcttataacaatctaactgctgcgatcgaagacctaaagcgtttgagggaggacaggtccaagttaaaaacaaaaacgatttaa

SEQ ID NO: 131 - protein, 506 aa, synthetic construct
MHLQPATTPLKIHFIPFISPGHIIPLSELARIFASRGEHVTIITTPFNADLLQKSIDEDRDSGEHIGIHTVEWSATELGLPHGIENLSNVTDLETAIKLHRALMLMQKQMEDFMTRNPPDCIIADTFYPWASEFANRMGIPRLIFYPWSTFALCLMESIRSPDSPHRRLSSDSDPFVVPGLPHPIILTRSQLPEHDRKDIADPAAQLMDQHKETEMKSYGIILNNFAEIETEYTEHYKKITGHKVWHIGPAAAIVHRNAKEKAERVFKSDEHDNNLVINWLNSKEPNSVVYVCFGSGCQFPDKQLYEIACGLELSGHQFVWVVRGKDKQIDVNDDGEKTWLPKGFEERMKTENKGLIVRGWAPQVLVLDHPSLGCFLTHCGWNSTIEGITAGVPLITWPVFAEQFYNEKLITQVHGNGVVVGSEEWIMLFTVAKSLVSRDKIENAVRKIMDGGDEAVQIRRRARELGEKAWKAASTGGSSYNNLTAAIEDLKRLREDRSKLKTKTI

SEQ ID NO: 132 - DNA, 1521 bp, synthetic construct
  source 1..1521: mol_type = other DNA
atgcacttgcagccggccactactcctctgaaaattcacttcatacctttcatatctccgggtcacatcatcccactctctgaattggctcgtatctttgcttcacgcggtgagcatgtgactatcattaccactcctttcaacgctgacctacttcagaaatccattgacgaagacagagattccggcgagcacattggcatccacaccgttgaatggtcagccacagagctaggccttcctcatgggattgaaaatctcagcaacgtcactgacttggagaccgccattaagctacacagggccctcatgctaatgcagaaacagatggaagatttcatgacccgaaatccacccgattgtataattgccgacacgttttacccgtgggcctccgaatttgctaatcggatgggtatcccgagactcattttctacccgtggagtactttcgcgctctgtttgatggaatctattcgatctcccgactccccgcaccgaagattgagttcggattcggatccatttgtggttccgggtcttcctcacccgatcatcttgacccgttctcagcttccggaacacgatcgaaaggacatagcggacccggctgcccaactcatggatcagcataaagaaactgagatgaagagctacggaattattctcaacaatttcgcggagatcgaaacagaatacacagagcattacaagaaaataacgggtcacaaggtttggcacattggacctgccgcagcaattgttcaccgaaatgccaaagagaaggcagagagggtattcaagagtgatgagcatgacaataaccttgtcatcaattggctcaactcgaaggaaccaaactcagttgtttatgtttgtttcggcagcggatgtcaattccctgataaacaactctatgagattgcatgcgggttagagttatctgggcatcaatttgtttgggtggttcgcggaaaagataaacaaatcgatgttaatgacgatggggagaagacatggttgcctaaagggtttgaggaaagaatgaaaacagaaaataaaggtttgattgtaaggggatgggccccacaggtgctggttttggatcatccatcattgggatgtttcttgacgcattgcggctggaattctacgattgagggaatcacagcaggcgttcctttgatcacgtggccagtattcgccgagcaattctataatgagaagctaatcacgcaggtgcatgggaatggggtggtggttggttcagaggagtggatcatgttgttcaccgtcgctaaaagcttggtaagtagagacaaaattgagaatgctgtgaggaagataatggacggtggtgatgaggctgtacaaatcagaaggcgggcccgggaacttggagaaaaagcttggaaagctgcttcaactggggggtcctcctacaataatctaaccgcagcaattgaagaccttaagcggttgagagaggaccgttcgaagctgaaaacaaaaacaatttga

SEQ ID NO: 133 - DNA, 1521 bp, synthetic construct
  source 1..1521: mol_type = other DNA
atgcaccttcaaccggcgaccacaccattgaagatacatttcatacccttcatttcacctggccacatcatccccctgagtgaattggcgcgtatattcgcgtccagaggcgagcacgtgacgataataacgacgccctttaacgcagatttgcttcaaaagtctatcgacgaggatagagattcaggtgaacatatcggcatacatactgtggagtggtccgcaactgaactgggtttgcctcatgggatcgagaatctaagcaacgtaaccgacctagagaccgccattaaacttcaccgtgcgctaatgttgatgcaaaagcaaatggaggacttcatgacgagaaatcctcctgactgtatcatagcagacactttttacccgtgggcgagtgaatttgcaaataggatgggcataccacgtcttatcttctacccgtggagcacatttgcgctgtgcctaatggaatcaatacgtagtcctgactccccacataggcgtctgtcatccgacagtgacccgttcgttgtgcctgggttaccacaccccataatccttaccaggtctcaactaccggagcacgacagaaaagacatcgctgatcctgccgcgcaattaatggaccagcataaagagaccgagatgaagtcttatggtataatcttaaacaattttgcggagattgaaacagagtataccgagcattacaaaaagattacggggcacaaggtttggcacataggtcctgctgctgctatagtccatcgtaacgccaaggagaaagcggagagggtttttaaaagcgacgagcatgacaacaatcttgttataaactggctaaatagcaaggaacctaactccgttgtctatgtctgcttcggctccgggtgtcaattccctgataaacaactttacgagattgcttgcggtctagagttgtccggtcatcagtttgtgtgggtggttaggggaaaagacaagcaaatcgacgtgaatgacgacggtgaaaagacttggctgccgaaaggatttgaggagaggatgaaaactgaaaacaaaggactaattgttcgtggatgggccccccaagttttagttctggatcatccaagccttggttgtttcctaacccattgcggctggaacagcacgatagaaggtataacagcgggggtaccgctgattacttggccagttttcgccgaacaattttataatgagaaacttattacccaggttcacggaaatggcgtagtcgttggttcagaggaatggattatgttgttcacagtcgcaaaatctctggtatccagggacaaaatagagaacgcagtcagaaaaatcatggacggaggcgacgaagcagtccagataaggcgtagggccagagaactaggtgagaaagcgtggaaggcagcttctaccggaggctcatcttataacaatctaactgctgcgatcgaagacctaaagcgtttgagggaggacaggtccaagttaaaaacaaaaacgatttaa

SEQ ID NO: 134 - protein, 503 aa, synthetic construct
MPATTPLKIHFIPFISPGHIIPLSELARIFASRGEHVTIITTPFNADLLQKSIDEDRDSGEHIGIHTVEWSATELGLPHGIENLSNVTDLETAIKLHRALMLMQKQMEDFMTRNPPDCIIADTFYPWASEFANRMGIPRLIFYPWSTFALCLMESIRSPDSPHRRLSSDSDPFVVPGLPHPIILTRSQLPEHDRKDIADPAAQLMDQHKETEMKSYGIILNNFAEIETEYTEHYKKITGHKVWHIGPAAAIVHRNAKEKAERVFKSDEHDNNLVINWLNSKEPNSVVYVCFGSGCQFPDKQLYEIACGLELSGHQFVWVVRGKDKQIDVNDDGEKTWLPKGFEERMKTENKGLIVRGWAPQVLVLDHPSLGCFLTHCGWNSTIEGITAGVPLITWPVFAEQFYNEKLITQVHGNGVVVGSEEWIMLFTVAKSLVSRDKIENAVRKIMDGGDEAVQIRRRARELGEKAWKAASTGGSSYNNLTAAIEDLKRLREDRSKLKTKTI

SEQ ID NO: 135 - DNA, 1512 bp, synthetic construct
  source 1..1512: mol_type = other DNA
atgccggccactactcctctgaaaattcacttcatacctttcatatctccgggtcacatcatcccactctctgaattggctcgtatctttgcttcacgcggtgagcatgtgactatcattaccactcctttcaacgctgacctacttcagaaatccattgacgaagacagagattccggcgagcacattggcatccacaccgttgaatggtcagccacagagctaggccttcctcatgggattgaaaatctcagcaacgtcactgacttggagaccgccattaagctacacagggccctcatgctaatgcagaaacagatggaagatttcatgacccgaaatccacccgattgtataattgccgacacgttttacccgtgggcctccgaatttgctaatcggatgggtatcccgagactcattttctacccgtggagtactttcgcgctctgtttgatggaatctattcgatctcccgactccccgcaccgaagattgagttcggattcggatccatttgtggttccgggtcttcctcacccgatcatcttgacccgttctcagcttccggaacacgatcgaaaggacatagcggacccggctgcccaactcatggatcagcataaagaaactgagatgaagagctacggaattattctcaacaatttcgcggagatcgaaacagaatacacagagcattacaagaaaataacgggtcacaaggtttggcacattggacctgccgcagcaattgttcaccgaaatgccaaagagaaggcagagagggtattcaagagtgatgagcatgacaataaccttgtcatcaattggctcaactcgaaggaaccaaactcagttgtttatgtttgtttcggcagcggatgtcaattccctgataaacaactctatgagattgcatgcgggttagagttatctgggcatcaatttgtttgggtggttcgcggaaaagataaacaaatcgatgttaatgacgatggggagaagacatggttgcctaaagggtttgaggaaagaatgaaaacagaaaataaaggtttgattgtaaggggatgggccccacaggtgctggttttggatcatccatcattgggatgtttcttgacgcattgcggctggaattctacgattgagggaatcacagcaggcgttcctttgatcacgtggccagtattcgccgagcaattctataatgagaagctaatcacgcaggtgcatgggaatggggtggtggttggttcagaggagtggatcatgttgttcaccgtcgctaaaagcttggtaagtagagacaaaattgagaatgctgtgaggaagataatggacggtggtgatgaggctgtacaaatcagaaggcgggcccgggaacttggagaaaaagcttggaaagctgcttcaactggggggtcctcctacaataatctaaccgcagcaattgaagaccttaagcggttgagagaggaccgttcgaagctgaaaacaaaaacaatttga

SEQ ID NO: 136 - DNA, 1512 bp, synthetic construct
  source 1..1512: mol_type = other DNA
atgccggcgaccacaccattgaagatacatttcatacccttcatttcacctggccacatcatccccctgagtgaattggcgcgtatattcgcgtccagaggcgagcacgtgacgataataacgacgccctttaacgcagatttgcttcaaaagtctatcgacgaggatagagattcaggtgaacatatcggcatacatactgtggagtggtccgcaactgaactgggtttgcctcatgggatcgagaatctaagcaacgtaaccgacctagagaccgccattaaacttcaccgtgcgctaatgttgatgcaaaagcaaatggaggacttcatgacgagaaatcctcctgactgtatcatagcagacactttttacccgtgggcgagtgaatttgcaaataggatgggcataccacgtcttatcttctacccgtggagcacatttgcgctgtgcctaatggaatcaatacgtagtcctgactccccacataggcgtctgtcatccgacagtgacccgttcgttgtgcctgggttaccacaccccataatccttaccaggtctcaactaccggagcacgacagaaaagacatcgctgatcctgccgcgcaattaatggaccagcataaagagaccgagatgaagtcttatggtataatcttaaacaattttgcggagattgaaacagagtataccgagcattacaaaaagattacggggcacaaggtttggcacataggtcctgctgctgctatagtccatcgtaacgccaaggagaaagcggagagggtttttaaaagcgacgagcatgacaacaatcttgttataaactggctaaatagcaaggaacctaactccgttgtctatgtctgcttcggctccgggtgtcaattccctgataaacaactttacgagattgcttgcggtctagagttgtccggtcatcagtttgtgtgggtggttaggggaaaagacaagcaaatcgacgtgaatgacgacggtgaaaagacttggctgccgaaaggatttgaggagaggatgaaaactgaaaacaaaggactaattgttcgtggatgggccccccaagttttagttctggatcatccaagccttggttgtttcctaacccattgcggctggaacagcacgatagaaggtataacagcgggggtaccgctgattacttggccagttttcgccgaacaattttataatgagaaacttattacccaggttcacggaaatggcgtagtcgttggttcagaggaatggattatgttgttcacagtcgcaaaatctctggtatccagggacaaaatagagaacgcagtcagaaaaatcatggacggaggcgacgaagcagtccagataaggcgtagggccagagaactaggtgagaaagcgtggaaggcagcttctaccggaggctcatcttataacaatctaactgctgcgatcgaagacctaaagcgtttgagggaggacaggtccaagttaaaaacaaaaacgatttaa

SEQ ID NO: 137 - protein, 500 aa, synthetic construct
MTPLKIHFIPFISPGHIIPLSELARIFASRGEHVTIITTPFNADLLQKSIDEDRDSGEHIGIHTVEWSATELGLPHGIENLSNVTDLETAIKLHRALMLMQKQMEDFMTRNPPDCIIADTFYPWASEFANRMGIPRLIFYPWSTFALCLMESIRSPDSPHRRLSSDSDPFVVPGLPHPIILTRSQLPEHDRKDIADPAAQLMDQHKETEMKSYGIILNNFAEIETEYTEHYKKITGHKVWHIGPAAAIVHRNAKEKAERVFKSDEHDNNLVINWLNSKEPNSVVYVCFGSGCQFPDKQLYEIACGLELSGHQFVWVVRGKDKQIDVNDDGEKTWLPKGFEERMKTENKGLIVRGWAPQVLVLDHPSLGCFLTHCGWNSTIEGITAGVPLITWPVFAEQFYNEKLITQVHGNGVVVGSEEWIMLFTVAKSLVSRDKIENAVRKIMDGGDEAVQIRRRARELGEKAWKAASTGGSSYNNLTAAIEDLKRLREDRSKLKTKTI

SEQ ID NO: 138 - DNA, 1503 bp, synthetic construct
  source 1..1503: mol_type = other DNA
atgactcctctgaaaattcacttcatacctttcatatctccgggtcacatcatcccactctctgaattggctcgtatctttgcttcacgcggtgagcatgtgactatcattaccactcctttcaacgctgacctacttcagaaatccattgacgaagacagagattccggcgagcacattggcatccacaccgttgaatggtcagccacagagctaggccttcctcatgggattgaaaatctcagcaacgtcactgacttggagaccgccattaagctacacagggccctcatgctaatgcagaaacagatggaagatttcatgacccgaaatccacccgattgtataattgccgacacgttttacccgtgggcctccgaatttgctaatcggatgggtatcccgagactcattttctacccgtggagtactttcgcgctctgtttgatggaatctattcgatctcccgactccccgcaccgaagattgagttcggattcggatccatttgtggttccgggtcttcctcacccgatcatcttgacccgttctcagcttccggaacacgatcgaaaggacatagcggacccggctgcccaactcatggatcagcataaagaaactgagatgaagagctacggaattattctcaacaatttcgcggagatcgaaacagaatacacagagcattacaagaaaataacgggtcacaaggtttggcacattggacctgccgcagcaattgttcaccgaaatgccaaagagaaggcagagagggtattcaagagtgatgagcatgacaataaccttgtcatcaattggctcaactcgaaggaaccaaactcagttgtttatgtttgtttcggcagcggatgtcaattccctgataaacaactctatgagattgcatgcgggttagagttatctgggcatcaatttgtttgggtggttcgcggaaaagataaacaaatcgatgttaatgacgatggggagaagacatggttgcctaaagggtttgaggaaagaatgaaaacagaaaataaaggtttgattgtaaggggatgggccccacaggtgctggttttggatcatccatcattgggatgtttcttgacgcattgcggctggaattctacgattgagggaatcacagcaggcgttcctttgatcacgtggccagtattcgccgagcaattctataatgagaagctaatcacgcaggtgcatgggaatggggtggtggttggttcagaggagtggatcatgttgttcaccgtcgctaaaagcttggtaagtagagacaaaattgagaatgctgtgaggaagataatggacggtggtgatgaggctgtacaaatcagaaggcgggcccgggaacttggagaaaaagcttggaaagctgcttcaactggggggtcctcctacaataatctaaccgcagcaattgaagaccttaagcggttgagagaggaccgttcgaagctgaaaacaaaaacaatttga

SEQ ID NO: 139 - DNA, 1503 bp, synthetic construct
  source 1..1503: mol_type = other DNA
atgacaccattgaagatacatttcatacccttcatttcacctggccacatcatccccctgagtgaattggcgcgtatattcgcgtccagaggcgagcacgtgacgataataacgacgccctttaacgcagatttgcttcaaaagtctatcgacgaggatagagattcaggtgaacatatcggcatacatactgtggagtggtccgcaactgaactgggtttgcctcatgggatcgagaatctaagcaacgtaaccgacctagagaccgccattaaacttcaccgtgcgctaatgttgatgcaaaagcaaatggaggacttcatgacgagaaatcctcctgactgtatcatagcagacactttttacccgtgggcgagtgaatttgcaaataggatgggcataccacgtcttatcttctacccgtggagcacatttgcgctgtgcctaatggaatcaatacgtagtcctgactccccacataggcgtctgtcatccgacagtgacccgttcgttgtgcctgggttaccacaccccataatccttaccaggtctcaactaccggagcacgacagaaaagacatcgctgatcctgccgcgcaattaatggaccagcataaagagaccgagatgaagtcttatggtataatcttaaacaattttgcggagattgaaacagagtataccgagcattacaaaaagattacggggcacaaggtttggcacataggtcctgctgctgctatagtccatcgtaacgccaaggagaaagcggagagggtttttaaaagcgacgagcatgacaacaatcttgttataaactggctaaatagcaaggaacctaactccgttgtctatgtctgcttcggctccgggtgtcaattccctgataaacaactttacgagattgcttgcggtctagagttgtccggtcatcagtttgtgtgggtggttaggggaaaagacaagcaaatcgacgtgaatgacgacggtgaaaagacttggctgccgaaaggatttgaggagaggatgaaaactgaaaacaaaggactaattgttcgtggatgggccccccaagttttagttctggatcatccaagccttggttgtttcctaacccattgcggctggaacagcacgatagaaggtataacagcgggggtaccgctgattacttggccagttttcgccgaacaattttataatgagaaacttattacccaggttcacggaaatggcgtagtcgttggttcagaggaatggattatgttgttcacagtcgcaaaatctctggtatccagggacaaaatagagaacgcagtcagaaaaatcatggacggaggcgacgaagcagtccagataaggcgtagggccagagaactaggtgagaaagcgtggaaggcagcttctaccggaggctcatcttataacaatctaactgctgcgatcgaagacctaaagcgtttgagggaggacaggtccaagttaaaaacaaaaacgatttaa

SEQ ID NO: 140 - protein, 497 aa, synthetic construct
MKIHFIPFISPGHIIPLSELARIFASRGEHVTIITTPFNADLLQKSIDEDRDSGEHIGIHTVEWSATELGLPHGIENLSNVTDLETAIKLHRALMLMQKQMEDFMTRNPPDCIIADTFYPWASEFANRMGIPRLIFYPWSTFALCLMESIRSPDSPHRRLSSDSDPFVVPGLPHPIILTRSQLPEHDRKDIADPAAQLMDQHKETEMKSYGIILNNFAEIETEYTEHYKKITGHKVWHIGPAAAIVHRNAKEKAERVFKSDEHDNNLVINWLNSKEPNSVVYVCFGSGCQFPDKQLYEIACGLELSGHQFVWVVRGKDKQIDVNDDGEKTWLPKGFEERMKTENKGLIVRGWAPQVLVLDHPSLGCFLTHCGWNSTIEGITAGVPLITWPVFAEQFYNEKLITQVHGNGVVVGSEEWIMLFTVAKSLVSRDKIENAVRKIMDGGDEAVQIRRRARELGEKAWKAASTGGSSYNNLTAAIEDLKRLREDRSKLKTKTI

SEQ ID NO: 141 - DNA, 1494 bp, synthetic construct
  source 1..1494: mol_type = other DNA
atgaaaattcacttcatacctttcatatctccgggtcacatcatcccactctctgaattggctcgtatctttgcttcacgcggtgagcatgtgactatcattaccactcctttcaacgctgacctacttcagaaatccattgacgaagacagagattccggcgagcacattggcatccacaccgttgaatggtcagccacagagctaggccttcctcatgggattgaaaatctcagcaacgtcactgacttggagaccgccattaagctacacagggccctcatgctaatgcagaaacagatggaagatttcatgacccgaaatccacccgattgtataattgccgacacgttttacccgtgggcctccgaatttgctaatcggatgggtatcccgagactcattttctacccgtggagtactttcgcgctctgtttgatggaatctattcgatctcccgactccccgcaccgaagattgagttcggattcggatccatttgtggttccgggtcttcctcacccgatcatcttgacccgttctcagcttccggaacacgatcgaaaggacatagcggacccggctgcccaactcatggatcagcataaagaaactgagatgaagagctacggaattattctcaacaatttcgcggagatcgaaacagaatacacagagcattacaagaaaataacgggtcacaaggtttggcacattggacctgccgcagcaattgttcaccgaaatgccaaagagaaggcagagagggtattcaagagtgatgagcatgacaataaccttgtcatcaattggctcaactcgaaggaaccaaactcagttgtttatgtttgtttcggcagcggatgtcaattccctgataaacaactctatgagattgcatgcgggttagagttatctgggcatcaatttgtttgggtggttcgcggaaaagataaacaaatcgatgttaatgacgatggggagaagacatggttgcctaaagggtttgaggaaagaatgaaaacagaaaataaaggtttgattgtaaggggatgggccccacaggtgctggttttggatcatccatcattgggatgtttcttgacgcattgcggctggaattctacgattgagggaatcacagcaggcgttcctttgatcacgtggccagtattcgccgagcaattctataatgagaagctaatcacgcaggtgcatgggaatggggtggtggttggttcagaggagtggatcatgttgttcaccgtcgctaaaagcttggtaagtagagacaaaattgagaatgctgtgaggaagataatggacggtggtgatgaggctgtacaaatcagaaggcgggcccgggaacttggagaaaaagcttggaaagctgcttcaactggggggtcctcctacaataatctaaccgcagcaattgaagaccttaagcggttgagagaggaccgttcgaagctgaaaacaaaaacaatttga

SEQ ID NO: 142 - DNA, 1494 bp, synthetic construct
  source 1..1494: mol_type = other DNA
atgaagatacatttcatacccttcatttcacctggccacatcatccccctgagtgaattggcgcgtatattcgcgtccagaggcgagcacgtgacgataataacgacgccctttaacgcagatttgcttcaaaagtctatcgacgaggatagagattcaggtgaacatatcggcatacatactgtggagtggtccgcaactgaactgggtttgcctcatgggatcgagaatctaagcaacgtaaccgacctagagaccgccattaaacttcaccgtgcgctaatgttgatgcaaaagcaaatggaggacttcatgacgagaaatcctcctgactgtatcatagcagacactttttacccgtgggcgagtgaatttgcaaataggatgggcataccacgtcttatcttctacccgtggagcacatttgcgctgtgcctaatggaatcaatacgtagtcctgactccccacataggcgtctgtcatccgacagtgacccgttcgttgtgcctgggttaccacaccccataatccttaccaggtctcaactaccggagcacgacagaaaagacatcgctgatcctgccgcgcaattaatggaccagcataaagagaccgagatgaagtcttatggtataatcttaaacaattttgcggagattgaaacagagtataccgagcattacaaaaagattacggggcacaaggtttggcacataggtcctgctgctgctatagtccatcgtaacgccaaggagaaagcggagagggtttttaaaagcgacgagcatgacaacaatcttgttataaactggctaaatagcaaggaacctaactccgttgtctatgtctgcttcggctccgggtgtcaattccctgataaacaactttacgagattgcttgcggtctagagttgtccggtcatcagtttgtgtgggtggttaggggaaaagacaagcaaatcgacgtgaatgacgacggtgaaaagacttggctgccgaaaggatttgaggagaggatgaaaactgaaaacaaaggactaattgttcgtggatgggccccccaagttttagttctggatcatccaagccttggttgtttcctaacccattgcggctggaacagcacgatagaaggtataacagcgggggtaccgctgattacttggccagttttcgccgaacaattttataatgagaaacttattacccaggttcacggaaatggcgtagtcgttggttcagaggaatggattatgttgttcacagtcgcaaaatctctggtatccagggacaaaatagagaacgcagtcagaaaaatcatggacggaggcgacgaagcagtccagataaggcgtagggccagagaactaggtgagaaagcgtggaaggcagcttctaccggaggctcatcttataacaatctaactgctgcgatcgaagacctaaagcgtttgagggaggacaggtccaagttaaaaacaaaaacgatttaa

SEQ ID NO: 143 - protein, 607 aa, synthetic construct
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMDSTHLQPATTPLKIHFIPFISPGHIIPLSELARIFASRGEHVTIITTPFNADLLQKSIDEDRDSGEHIGIHTVEWSATELGLPHGIENLSNVTDLETAIKLHRALMLMQKQMEDFMTRNPPDCIIADTFYPWASEFANRMGIPRLIFYPWSTFALCLMESIRSPDSPHRRLSSDSDPFVVPGLPHPIILTRSQLPEHDRKDIADPAAQLMDQHKETEMKSYGIILNNFAEIETEYTEHYKKITGHKVWHIGPAAAIVHRNAKEKAERVFKSDEHDNNLVINWLNSKEPNSVVYVCFGSGCQFPDKQLYEIACGLELSGHQFVWVVRGKDKQIDVNDDGEKTWLPKGFEERMKTENKGLIVRGWAPQVLVLDHPSLGCFLTHCGWNSTIEGITAGVPLITWPVFAEQFYNEKLITQVHGNGVVVGSEEWIMLFTVAKSLVSRDKIENAVRKIMDGGDEAVQIRRRARELGEKAWKAASTGGSSYNNLTAAIEDLKRLREDRSKLKTKTI

SEQ ID NO: 144 - DNA, 1824 bp, synthetic construct
  source 1..1824: mol_type = other DNA
atgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtggtatggatagtacacaccttcaaccggcgaccacaccattgaagatacatttcatacccttcatttcacctggccacatcatccccctgagtgaattggcgcgtatattcgcgtccagaggcgagcacgtgacgataataacgacgccctttaacgcagatttgcttcaaaagtctatcgacgaggatagagattcaggtgaacatatcggcatacatactgtggagtggtccgcaactgaactgggtttgcctcatgggatcgagaatctaagcaacgtaaccgacctagagaccgccattaaacttcaccgtgcgctaatgttgatgcaaaagcaaatggaggacttcatgacgagaaatcctcctgactgtatcatagcagacactttttacccgtgggcgagtgaatttgcaaataggatgggcataccacgtcttatcttctacccgtggagcacatttgcgctgtgcctaatggaatcaatacgtagtcctgactccccacataggcgtctgtcatccgacagtgacccgttcgttgtgcctgggttaccacaccccataatccttaccaggtctcaactaccggagcacgacagaaaagacatcgctgatcctgccgcgcaattaatggaccagcataaagagaccgagatgaagtcttatggtataatcttaaacaattttgcggagattgaaacagagtataccgagcattacaaaaagattacggggcacaaggtttggcacataggtcctgctgctgctatagtccatcgtaacgccaaggagaaagcggagagggtttttaaaagcgacgagcatgacaacaatcttgttataaactggctaaatagcaaggaacctaactccgttgtctatgtctgcttcggctccgggtgtcaattccctgataaacaactttacgagattgcttgcggtctagagttgtccggtcatcagtttgtgtgggtggttaggggaaaagacaagcaaatcgacgtgaatgacgacggtgaaaagacttggctgccgaaaggatttgaggagaggatgaaaactgaaaacaaaggactaattgttcgtggatgggccccccaagttttagttctggatcatccaagccttggttgtttcctaacccattgcggctggaacagcacgatagaaggtataacagcgggggtaccgctgattacttggccagttttcgccgaacaattttataatgagaaacttattacccaggttcacggaaatggcgtagtcgttggttcagaggaatggattatgttgttcacagtcgcaaaatctctggtatccagggacaaaatagagaacgcagtcagaaaaatcatggacggaggcgacgaagcagtccagataaggcgtagggccagagaactaggtgagaaagcgtggaaggcagcttctaccggaggctcatcttataacaatctaactgctgcgatcgaagacctaaagcgtttgagggaggacaggtccaagttaaaaacaaaaacgatttaa

SEQ ID NO: 145 - protein, 633 aa, synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLADYDIPTTENLYFQGHMDSTHLQPATTPLKIHFIPFISPGHIIPLSELARIFASRGEHVTIITTPFNADLLQKSIDEDRDSGEHIGIHTVEWSATELGLPHGIENLSNVTDLETAIKLHRALMLMQKQMEDFMTRNPPDCIIADTFYPWASEFANRMGIPRLIFYPWSTFALCLMESIRSPDSPHRRLSSDSDPFVVPGLPHPIILTRSQLPEHDRKDIADPAAQLMDQHKETEMKSYGIILNNFAEIETEYTEHYKKITGHKVWHIGPAAAIVHRNAKEKAERVFKSDEHDNNLVINWLNSKEPNSVVYVCFGSGCQFPDKQLYEIACGLELSGHQFVWVVRGKDKQIDVNDDGEKTWLPKGFEERMKTENKGLIVRGWAPQVLVLDHPSLGCFLTHCGWNSTIEGITAGVPLITWPVFAEQFYNEKLITQVHGNGVVVGSEEWIMLFTVAKSLVSRDKIENAVRKIMDGGDEAVQIRRRARELGEKAWKAASTGGSSYNNLTAAIEDLKRLREDRSKLKTKTI

SEQ ID NO: 146 - DNA, 1902 bp, synthetic construct
  source 1..1902: mol_type = other DNA
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcggattacgatatcccaacgaccgaaaacctttacttccagggccatatggatagtacacaccttcaaccggcgaccacaccattgaagatacatttcatacccttcatttcacctggccacatcatccccctgagtgaattggcgcgtatattcgcgtccagaggcgagcacgtgacgataataacgacgccctttaacgcagatttgcttcaaaagtctatcgacgaggatagagattcaggtgaacatatcggcatacatactgtggagtggtccgcaactgaactgggtttgcctcatgggatcgagaatctaagcaacgtaaccgacctagagaccgccattaaacttcaccgtgcgctaatgttgatgcaaaagcaaatggaggacttcatgacgagaaatcctcctgactgtatcatagcagacactttttacccgtgggcgagtgaatttgcaaataggatgggcataccacgtcttatcttctacccgtggagcacatttgcgctgtgcctaatggaatcaatacgtagtcctgactccccacataggcgtctgtcatccgacagtgacccgttcgttgtgcctgggttaccacaccccataatccttaccaggtctcaactaccggagcacgacagaaaagacatcgctgatcctgccgcgcaattaatggaccagcataaagagaccgagatgaagtcttatggtataatcttaaacaattttgcggagattgaaacagagtataccgagcattacaaaaagattacggggcacaaggtttggcacataggtcctgctgctgctatagtccatcgtaacgccaaggagaaagcggagagggtttttaaaagcgacgagcatgacaacaatcttgttataaactggctaaatagcaaggaacctaactccgttgtctatgtctgcttcggctccgggtgtcaattccctgataaacaactttacgagattgcttgcggtctagagttgtccggtcatcagtttgtgtgggtggttaggggaaaagacaagcaaatcgacgtgaatgacgacggtgaaaagacttggctgccgaaaggatttgaggagaggatgaaaactgaaaacaaaggactaattgttcgtggatgggccccccaagttttagttctggatcatccaagccttggttgtttcctaacccattgcggctggaacagcacgatagaaggtataacagcgggggtaccgctgattacttggccagttttcgccgaacaattttataatgagaaacttattacccaggttcacggaaatggcgtagtcgttggttcagaggaatggattatgttgttcacagtcgcaaaatctctggtatccagggacaaaatagagaacgcagtcagaaaaatcatggacggaggcgacgaagcagtccagataaggcgtagggccagagaactaggtgagaaagcgtggaaggcagcttctaccggaggctcatcttataacaatctaactgctgcgatcgaagacctaaagcgtttgagggaggacaggtccaagttaaaaacaaaaacgatttaa

SEQ ID NO: 147 - protein, 891 aa, synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTDYDIPTTENLYFQGHMDSTHLQPATTPLKIHFIPFISPGHIIPLSELARIFASRGEHVTIITTPFNADLLQKSIDEDRDSGEHIGIHTVEWSATELGLPHGIENLSNVTDLETAIKLHRALMLMQKQMEDFMTRNPPDCIIADTFYPWASEFANRMGIPRLIFYPWSTFALCLMESIRSPDSPHRRLSSDSDPFVVPGLPHPIILTRSQLPEHDRKDIADPAAQLMDQHKETEMKSYGIILNNFAEIETEYTEHYKKITGHKVWHIGPAAAIVHRNAKEKAERVFKSDEHDNNLVINWLNSKEPNSVVYVCFGSGCQFPDKQLYEIACGLELSGHQFVWVVRGKDKQIDVNDDGEKTWLPKGFEERMKTENKGLIVRGWAPQVLVLDHPSLGCFLTHCGWNSTIEGITAGVPLITWPVFAEQFYNEKLITQVHGNGVVVGSEEWIMLFTVAKSLVSRDKIENAVRKIMDGGDEAVQIRRRARELGEKAWKAASTGGSSYNNLTAAIEDLKRLREDRSKLKTKTI

SEQ ID NO: 148 - DNA, 2673 bp, synthetic construct
  source 1..2673: mol_type = other DNA
aaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactgattacgatatcccaacgaccgaaaacctttacttccagggccatatggatagtacacaccttcaaccggcgaccacaccattgaagatacatttcatacccttcatttcacctggccacatcatccccctgagtgaattggcgcgtatattcgcgtccagaggcgagcacgtgacgataataacgacgccctttaacgcagatttgcttcaaaagtctatcgacgaggatagagattcaggtgaacatatcggcatacatactgtggagtggtccgcaactgaactgggtttgcctcatgggatcgagaatctaagcaacgtaaccgacctagagaccgccattaaacttcaccgtgcgctaatgttgatgcaaaagcaaatggaggacttcatgacgagaaatcctcctgactgtatcatagcagacactttttacccgtgggcgagtgaatttgcaaataggatgggcataccacgtcttatcttctacccgtggagcacatttgcgctgtgcctaatggaatcaatacgtagtcctgactccccacataggcgtctgtcatccgacagtgacccgttcgttgtgcctgggttaccacaccccataatccttaccaggtctcaactaccggagcacgacagaaaagacatcgctgatcctgccgcgcaattaatggaccagcataaagagaccgagatgaagtcttatggtataatcttaaacaattttgcggagattgaaacagagtataccgagcattacaaaaagattacggggcacaaggtttggcacataggtcctgctgctgctatagtccatcgtaacgccaaggagaaagcggagagggtttttaaaagcgacgagcatgacaacaatcttgttataaactggctaaatagcaaggaacctaactccgttgtctatgtctgcttcggctccgggtgtcaattccctgataaacaactttacgagattgcttgcggtctagagttgtccggtcatcagtttgtgtgggtggttaggggaaaagacaagcaaatcgacgtgaatgacgacggtgaaaagacttggctgccgaaaggatttgaggagaggatgaaaactgaaaacaaaggactaattgttcgtggatgggccccccaagttttagttctggatcatccaagccttggttgtttcctaacccattgcggctggaacagcacgatagaaggtataacagcgggggtaccgctgattacttggccagttttcgccgaacaattttataatgagaaacttattacccaggttcacggaaatggcgtagtcgttggttcagaggaatggattatgttgttcacagtcgcaaaatctctggtatccagggacaaaatagagaacgcagtcagaaaaatcatggacggaggcgacgaagcagtccagataaggcgtagggccagagaactaggtgagaaagcgtggaaggcagcttctaccggaggctcatcttataacaatctaactgctgcgatcgaagacctaaagcgtttgagggaggacaggtccaagttaaaaacaaaaacgatttaa

SEQ ID NO: 149 - protein, 988 aa, synthetic construct
MAAAAPNHRLHIAFFPWLAFGHINPFFELAKLIAQKGHHISFISTPRNIQRLSQVPPQLADSIDLVSLPVIHNSNLPENAESTMDIPPDKTPYLGMLHDSLKEPLTQFLQTHSPDWILYDFSAGWLAAIVEDLGISHGYFSIIPCWNIGFNGRQMNGFQKPDISLPSAVSLKKYEVKKIMDLVKSFPKILDESATKSIASHSTCEVIFIRNCPEIEADWFDYTSKIFDKPVVPVGVVPPSVHITNKEKDEHFNKWLEIKEWLDQQDRGSVIYIAFGTESLPNQDEITMLAQGLELCGLPFFWALRKSNVASDQPNSDSVELPEGFEEQTKGRGIVWTSWAPQQRILGHNSIGGFVTHCGWSSVIEGIHYGRPLIMFPLTVEQSLNARILGEKKLGMEVPREDDGSFTGEVVAETLKLVLLDQDGKVYRDKVTEMSKVFGDKDKHEKYMGDLLEFFKNYRSLKRNGGGGSGGGGSGGGGSMDSTHLQPATTPLKIHFIPFISPGHIIPLSELARIFASRGEHVTIITTPFNADLLQKSIDEDRDSGEHIGIHTVEWSATELGLPHGIENLSNVTDLETAIKLHRALMLMQKQMEDFMTRNPPDCIIADTFYPWASEFANRMGIPRLIFYPWSTFALCLMESIRSPDSPHRRLSSDSDPFVVPGLPHPIILTRSQLPEHDRKDIADPAAQLMDQHKETEMKSYGIILNNFAEIETEYTEHYKKITGHKVWHIGPAAAIVHRNAKEKAERVFKSDEHDNNLVINWLNSKEPNSVVYVCFGSGCQFPDKQLYEIACGLELSGHQFVWVVRGKDKQIDVNDDGEKTWLPKGFEERMKTENKGLIVRGWAPQVLVLDHPSLGCFLTHCGWNSTIEGITAGVPLITWPVFAEQFYNEKLITQVHGNGVVVGSEEWIMLFTVAKSLVSRDKIENAVRKIMDGGDEAVQIRRRARELGEKAWKAASTGGSSYNNLTAAIEDLKRLREDRSKLKTKTI

SEQ ID NO: 150 - DNA, 2967 bp, synthetic construct
  source 1..2967: mol_type = other DNA
atggcagcagcggcacccaatcatcgtctgcacatagcgtttttcccctggctagctttcggccacatcaatccatttttcgaactagcaaagttaatcgcgcaaaaaggtcatcacatcagtttcatctccactcccagaaatatccagaggttaagtcaggttccgcctcagctagctgactccatcgatctggtatccttgcccgtaatacataactcaaatcttcccgaaaatgccgagtcaacaatggacatcccgccagacaaaaccccttaccttgggatgttgcacgactccttgaaagagccactaactcaatttcttcaaacacactccccggattggattttatatgatttctcagctggatggttggcagctatagtggaggatcttgggatcagtcacggttatttctcaataataccctgttggaacatcggctttaatgggagacagatgaacggttttcaaaaacccgatatcagtctaccttcagccgtttcactgaagaaatacgaggtcaagaaaattatggatctagtaaaaagtttcccgaaaattctggatgaaagcgccacgaaaagtatcgctagccactcaacctgtgaagtcatatttatcaggaattgccctgaaattgaagccgactggttcgattatacatcaaagatattcgacaagcccgtagtgcctgttggcgtggtgcctccgtcagtccatattaccaacaaggaaaaggatgagcattttaacaaatggttagaaataaaagagtggctggaccaacaggataggggtagtgtcatatatatcgcgtttggtactgagtccttacccaatcaagatgaaataactatgctagctcagggactggaactttgtggcctgccgtttttctgggcgttgaggaagtcaaacgttgcctcagatcagccgaactctgacagtgtggaattgccggagggcttcgaggaacaaactaagggaagagggattgtctggacctcatgggcaccacaacagagaatcctaggccataatagtatcggtgggtttgttactcattgtggctggtccagcgtaattgaagggatacactacggtaggcctttgataatgttcccgctaaccgttgaacaatctctaaatgctaggatactgggagagaaaaagttggggatggaggtaccgcgtgaggatgacggttcatttactggagaagtagttgccgagacattgaaacttgttttgttggaccaagacggaaaggtttaccgtgacaaggtaacagagatgtcaaaagtgttcggtgacaaagataaacatgaaaagtacatgggggacctactggagttctttaagaattataggtctctgaaacgtaacggcggcggcggttcaggaggagggggttccggaggggggggttctatggatagtacacaccttcaaccggcgaccacaccattgaagatacatttcatacccttcatttcacctggccacatcatccccctgagtgaattggcgcgtatattcgcgtccagaggcgagcacgtgacgataataacgacgccctttaacgcagatttgcttcaaaagtctatcgacgaggatagagattcaggtgaacatatcggcatacatactgtggagtggtccgcaactgaactgggtttgcctcatgggatcgagaatctaagcaacgtaaccgacctagagaccgccattaaacttcaccgtgcgctaatgttgatgcaaaagcaaatggaggacttcatgacgagaaatcctcctgactgtatcatagcagacactttttacccgtgggcgagtgaatttgcaaataggatgggcataccacgtcttatcttctacccgtggagcacatttgcgctgtgcctaatggaatcaatacgtagtcctgactccccacataggcgtctgtcatccgacagtgacccgttcgttgtgcctgggttaccacaccccataatccttaccaggtctcaactaccggagcacgacagaaaagacatcgctgatcctgccgcgcaattaatggaccagcataaagagaccgagatgaagtcttatggtataatcttaaacaattttgcggagattgaaacagagtataccgagcattacaaaaagattacggggcacaaggtttggcacataggtcctgctgctgctatagtccatcgtaacgccaaggagaaagcggagagggtttttaaaagcgacgagcatgacaacaatcttgttataaactggctaaatagcaaggaacctaactccgttgtctatgtctgcttcggctccgggtgtcaattccctgataaacaactttacgagattgcttgcggtctagagttgtccggtcatcagtttgtgtgggtggttaggggaaaagacaagcaaatcgacgtgaatgacgacggtgaaaagacttggctgccgaaaggatttgaggagaggatgaaaactgaaaacaaaggactaattgttcgtggatgggccccccaagttttagttctggatcatccaagccttggttgtttcctaacccattgcggctggaacagcacgatagaaggtataacagcgggggtaccgctgattacttggccagttttcgccgaacaattttataatgagaaacttattacccaggttcacggaaatggcgtagtcgttggttcagaggaatggattatgttgttcacagtcgcaaaatctctggtatccagggacaaaatagagaacgcagtcagaaaaatcatggacggaggcgacgaagcagtccagataaggcgtagggccagagaactaggtgagaaagcgtggaaggcagcttctaccggaggctcatcttataacaatctaactgctgcgatcgaagacctaaagcgtttgagggaggacaggtccaagttaaaaacaaaaacgatttaa

SEQ ID NO: 151 - protein, 509 aa, Quillaja saponaria
MDSTHLQPATTPLKIHVIPFIAPGHIIPLSELARIFASRGEHVTIITTPFNADLLQKSIDEDRDSGEHIGIHTVEWSATELGLPHGVENLSNVTDLETGIKLHRALVLMQKQMEDFMTRNPPDCIIADTFYPWASEFANRMGIPRLIFFPGCTFALCLMESIRSPDSPHRRLSSDSDPFVVPGLPHPIILTRSQLPEHDREDIADPAAQFMDQCKEAAMKSYGIILNNFAEIETEYTEHYKKITGHKVWHIGPAAAIVHRNAKEKAEGLFKSDEHDNNLVINWLNSKEPNSVVYVCFGSGCQFPDKQLYEIACGLELSGHQFIWVVRGKDKQIDVNDDEEKTWLPKGFEERMKTENKGLIVRGWAPQVLVLDHPSLGCFLTHCGWNSTIEGITAGVPLITWPVYSEQFYNEKLITQVHGNGVGVGSEEWIMLFSVAKSLVSRDKIENAVRKIMDGGDEALEIRRRARELGEKARKAASIGGSSDNNLTAAIEDLKRLREDRSKLKTKTI

SEQ ID NO: 152 - DNA, 1530 bp, Quillaja saponaria
  source 1..1530: mol_type = genomic DNA
atggactccacccacttgcagccggccactactcctctgaaaattcacgtcatacctttcatagctccgggtcacattatcccactctctgaattggctcgtatctttgcttcacgcggtgagcatgtgactatcataaccactcctttcaacgctgacctacttcagaaatccattgacgaagacagagattccggcgagcacattggcatccacaccgttgaatggtcagccacagagctaggccttcctcatggggttgaaaatctcagcaacgtcactgacttggagaccggcattaagctacacagggccctcgtgctaatgcagaaacagatggaagatttcatgacccgaaatccacccgattgtataattgccgacacgttttacccgtgggcctccgaatttgctaatcggatgggtatcccgagactcattttcttcccggggtgtactttcgcgctctgtttgatggaatctattcgatctcccgactccccgcaccgaagattgagttcggattcggacccatttgtggttccgggtcttcctcacccgatcatcttgacccgttctcaacttccggaacacgatcgagaggacatagcggacccggctgcccaattcatggatcagtgtaaagaagctgcgatgaagagctacggaattattctcaacaatttcgcggagatcgaaacagaatacacagagcattacaagaaaataacgggtcacaaggtttggcacattggacctgccgcagcaattgttcaccgaaatgccaaagagaaggcagaggggcttttcaaaagtgacgagcatgacaataaccttgtcatcaattggctcaactcgaaggaaccaaactcagttgtttatgtttgtttcggcagcggatgtcaattccctgataaacaactctatgagattgcatgcgggttagagttatctgggcatcaatttatttgggtggttcgcggcaaagataaacaaatcgatgttaatgacgatgaggagaagacatggttgcctaaagggtttgaggaaagaatgaaaacagaaaataaaggtttgattgtaaggggatgggccccacaggtgctggttttggatcatccatcgttgggatgtttcttgacgcattgcggctggaattctacgattgagggaatcacagcaggtgttcctttgatcacgtggccagtatactccgagcaattctataatgagaagctaatcacgcaggtgcatggtaatggggtgggggttggttcagaggagtggatcatgctgttcagcgtcgctaaaagtttggtaagtagagacaaaattgagaatgctgtgaggaagataatggacggtggtgatgaggctttagaaatcagaaggcgggcccgggaacttggagaaaaagctaggaaagctgcttcaattggggggtcctccgacaataatctaaccgcagcaattgaagaccttaagcggttgagagaggaccgttcgaagctgaaaacaaaaacaatttga

SEQ ID NO: 153 - DNA, 1530 bp, synthetic construct
  source 1..1530: mol_type = other DNA
atggactcaactcaccttcaaccggctaccacgccactgaaaatacatgtgattccatttattgcccctgggcatataatacctctttctgagctggcacgtattttcgcctccagaggtgagcacgttacaattattactacgccttttaacgccgacttgcttcaaaaatccattgatgaagacagagatagtggggagcacattggcattcatactgttgagtggtccgcgacggagttaggcctgccccatggagtcgaaaatcttagtaatgtcacggacctggagactgggattaaattgcaccgtgcgttagtattaatgcaaaagcaaatggaagactttatgacccgtaatcctccggactgcatcattgcggatacattctacccttgggccagcgaatttgcgaaccgtatgggtataccgcgtcttatattttttcctggctgtactttcgcgttatgtttgatggagtccataagaagtccggacagcccacatcgtagattaagcagcgattcagatccgttcgttgtaccgggtctgccacatccaataatcctaacgagaagtcagctacctgaacacgacagggaggatatcgcagatcctgcagcacaatttatggaccaatgtaaagaggcagctatgaagtcatatggaatcatcttgaacaattttgcggaaattgaaacagaatatactgagcactacaagaagataacaggacataaggtatggcacattgggcctgcagcggcaatcgtgcatagaaacgcaaaagaaaaagcggaaggtctttttaagtccgatgagcacgacaacaatcttgtaattaattggctaaactcaaaggagcctaactcagttgtctacgtgtgcttcggttctggatgccagttccctgataagcaactttacgaaatagcgtgtggcctggagctttccggacaccaattcatctgggtggtaagagggaaggacaaacagattgatgtgaatgatgatgaagaaaagacgtggctgcccaaaggtttcgaggaacgtatgaagacagaaaacaaaggtctaatagtaagggggtgggcaccccaggtgcttgtacttgaccatcctagcttagggtgctttttaacacattgtggctggaactctacgattgagggtattaccgcgggcgtcccgttaataacgtggcctgtttattccgaacagttttataacgaaaaactgattactcaagttcatgggaatggagtgggcgtaggatccgaggaatggataatgctattttcagtagctaagtcattggtctctcgtgataaaatcgagaatgcagtgagaaaaataatggatggaggggacgaggccttggagataaggcgtagggccagggagcttggtgagaaggcaaggaaagccgcctcaatcggcggatcatccgacaataacttaaccgcggccatagaagacttgaagaggttaagggaggataggagtaagcttaagacaaaaacgatttaa

SEQ ID NO: 154 - protein, 478 aa, Synechococcus sp.
MTIRRICCIGAGYVGGPTMAVIADRCPDVQVIVVDLNDQRIAAWNDADLGRLPVYEPGLDAVVGRARGRNLFFSTDVDGAIGEADMVFISVNTPTKVKGLGAGQASDLRWVEACARQVAKAATGHTIVVEKSTLPVRTAEAIKTILEAAQDPPGNGDVPRTFSVLSNPEFLAEGTAIRDLETPDRVLIGGEDERSIQSLAAVYGQWVPDDRILRTNLWSSELSKLTANAFLAQRISSINSVAALCESTGADVREVARAIGTDSRIGPKFLQSGPGFGGSCFQKDILNLVYLCRHFGLPEVADYWESVVHLNTWQQHRISRLVVQRLFGTVTGKRLAILGFAFKADTNDTREAPAIRISRDLLEEGAQLAIHDPKVEPSQISRDLRLESVVPDPETGTTSQALSGQGSWWTAGSVEQAVAGADAVLILTEWTLYRHLDWKDLAQRMRRPAWVFDARSVVDRAEVQSAGLTYWRVGDGAQ

SEQ ID NO: 155 - DNA, 1437 bp, Synechococcus sp.
  source 1..1437: mol_type = genomic DNA
atgacgattcggcggatctgctgcattggagccggttacgtcggtggaccgaccatggccgtgattgccgatcgttgtcctgatgtccaggtcatcgtggtggacctcaatgatcagcgcatcgcggcctggaatgacgcggacctcggtcgtttgcctgtctatgagcctggcctcgatgcggtggtgggtcgtgcccgtgggcgcaaccttttcttttccacggacgtggatggggcgattggggaggcggacatggtgttcatctccgtcaacacacccaccaaggtgaagggtttaggggcaggccaggccagtgatctgcgctgggttgaggcttgtgcccgtcaggtggccaaggcagccacaggccacacgatcgtcgtggaaaagagcacattgccggtccgcaccgccgaagccatcaaaacaattctggaagcggctcaggatccacctggcaacggtgatgtgccgcgcaccttctccgtactctccaatcccgagttcctggccgagggcacagcgatcagggatcttgagacgccggatcgggtcttgatcggcggggaggacgaacgatccatccagtccttggcagcggtctatgggcaatgggttccggacgatcgcattctgcgcaccaatctctggagcagtgaactctccaaactcaccgccaacgcctttctggcccagcggatcagctcgatcaattcggtggcagccctctgtgagtccactggtgctgatgtgcgggaggttgcgcgcgccatcggtacggacagtcgcattggtccgaaatttcttcagtccggtccgggatttggggggagttgtttccagaaggacatcctcaacctggtgtatctctgccgccatttcggactgcctgaagtcgctgattactgggaaagcgtggtccatctcaatacctggcagcagcaccggatctcgcgtttggtcgttcagcggttgttcggcacggtgaccggaaagcgcttggcgattctgggtttcgctttcaaagcggatacgaatgacactcgcgaggcgcctgccattcgaatttcccgcgatcttcttgaggaaggtgcgcagctggcgatccatgatcccaaggtggaaccctctcagatttcccgcgatcttcgactcgaatcggtggtgcccgatccagagacgggaaccacatcccaggccttgagtggccagggaagctggtggaccgctggaagtgtcgaacaggccgtggctggggccgatgcggtgctgatcctcaccgagtggactctctaccgccatctggactggaaggatctggcccaacgcatgcggcgtcccgcctgggtctttgatgcccgttccgttgtcgatcgggctgaggttcagtccgccgggctgacctactggcgcgtgggtgacggcgctcagtga

SEQ ID NO: 156 - DNA, 1437 bp, synthetic construct
  source 1..1437: mol_type = other DNA
atgaccatcaggcgtatctgttgcattggtgcaggttacgtaggcgggcctacaatggctgttatcgccgaccgttgtcctgacgtgcaggtcattgtcgtcgatctaaacgatcagagaatagctgcatggaacgatgctgatctagggaggctaccggtctacgagccaggtctagacgcagtcgtgggtcgtgctcgtggaagaaacttgttcttcagtacagatgtcgacggtgccatcggagaagctgatatggtctttatctccgtcaacacgccgacgaaagtcaaaggtcttggagcggggcaggcgagtgacttgcgttgggtagaggcttgcgctcgtcaggtagcgaaagcggccaccgggcacaccattgtagtggaaaagtctacattaccagtgaggaccgcagaggcgattaaaacaatcctggaagcggcccaagatccccctggtaacggggatgtgccaagaacattttcagtgctaagtaatcccgagtttttagcagaaggtacagcgataagagaccttgaaacaccggaccgtgtcctaattggcggagaggacgagagatcaatacagtccttggctgccgtgtacggccagtgggttcctgacgacaggattttgaggactaatctatggtcttctgaactgtctaagctaaccgccaatgcgttcttggcgcaacgtatctcatcaattaactccgttgcagccctttgcgaaagtactggggctgacgttagggaggttgcgcgtgcaattggtacagactcacgtatcggccctaaatttctgcagtctggccctgggttcggaggaagttgtttccagaaggacatcttgaatttggtgtatctatgcagacactttggattgccggaggtcgctgattactgggaaagcgtcgtgcacttaaacacatggcaacaacacagaataagtcgtttggttgtccaaaggctttttgggacagtcaccggaaaaaggttggcaatccttggatttgccttcaaagccgacactaatgatacaagagaagcaccagcaattcgtatttccagggacctgctagaggagggcgctcaattagcgatccatgatccaaaggttgagccgagccagatatcccgtgacctgagattagaaagcgtcgttcctgatcctgagacaggtacgaccagccaggcgttgtctggacaagggtcatggtggaccgcgggaagcgtggaacaggcggtagcaggagcggatgcggtgcttatcttgaccgaatggaccttatacagacacttagactggaaggatttggcccagaggatgcgtaggcccgcatgggtttttgatgccaggagtgttgtcgacagggcggaggttcaatctgccggactgacatactggcgtgtgggtgatggagcacaatga

SEQ ID NO: 157 - protein, 494 aa, synthetic construct
MFEIKKICCIGAGYVGGPTCSVIAHMCPEIRVTVVDVNESRINAWNSPTLPIYEPGLKEVVESCRGKNLFFSTNIDDAIKEADLVFISVNTPTKTYGMGKGRALDLKYIEACARRIVQNSNGYKIVTEKSTVPVRAAESIRRIFDANTKPNLNLQVLSNPEFLAEGTAIKDLKNPDRVLIGGDETPEGQRAVQALCAVYEHWVPREKILTTNTWSSELSKLAANAFLAQRISSINSISALCEATGADVEEVATAIGMDQRIGNKFLKASVGFGGSCFQKDVLNLVYLCEALNLPEVARYWQQVIDMNDYQRRRFASRIIDSLFNTVTDKKIAILGFAFKKDTGDTRESSSIYISKYLMDEGAHLHIYDPKVPREQIVVDLSHPGVSEDDQVSRLVTISKDPYEACDGAHAVVICTEWDMFKELDYERIHKKMLKPAFIFDGRRVLDGLHNELQTIGFQIETIGKKVSSKRIPYAPSGEIPKFSLQDPPNKKPKV

SEQ ID NO: 158 - DNA, 1485 bp, synthetic construct
  source 1..1485: mol_type = other DNA
atgtttgaaattaagaagatctgttgcatcggtgcaggctatgttggaggacccacatgtagtgtcattgctcatatgtgtcctgaaatcagggtaacggttgttgatgtcaatgaatcaagaatcaatgcgtggaattctcctacacttcctatttatgagccaggactaaaagaagtggtagaatcctgtcgaggaaaaaatctttttttttctaccaatattgatgatgccatcaaagaagctgatcttgtatttatttctgtgaatactccaacaaaaacctatggaatggggaaaggccgggcagcagatctgaagtatattgaagcttgtgctagacgcattgtgcaaaactcaaatgggtacaaaattgtgactgagaaaagcacagttccagtgcgggcagcagaaagtatccgtcgcatatttgatgcaaacacaaaacccaacttgaatttacaggtgctgtccaaccctgagtttctggcagagggaacagccatcaaggacctaaagaacccagacagagtactgattggaggggatgaaactccagagggccagagagctgtgcaggccctgtgtgctgtatatgagcactgggttcccagagaaaagatcctcaccactaatacttggtcttcagagctttccaaactggcagcaaatgcttttcttgcccagagaataagcagcattaactccataagtgctctgtgtgaagcaacaggagctgatgtagaagaggtagcaacagcgattggaatggaccagagaattggaaacaagtttctaaaagccagtgttgggtttggtgggagctgtttccaaaaggatgttctgaatttggtttatctctgtgaggctctgaatttgccagaagtagctcgttattggcagcaggtcatagacatgaatgactaccagaggaggaggtttgcttcccggatcatagatagtctgtttaatacagtaactgataagaagatagctattttgggatttgcattcaaaaaggacactggtgatacaagagaatcttctagtatatatattagcaaatatttgatggatgaaggtgcacatctacatatatatgatccaaaagtacctagggaacaaatagttgtggatctttctcatccaggtgtttcagaggatgaccaagtgtcccggctcgtgaccatttccaaggatccatatgaagcatgtgatggtgcccatgctgttgttatttgcactgagtgggacatgtttaaggaattggattatgaacgcattcataaaaaaatgctaaagccagcctttatcttcgatggacggcgtgtcctggatgggctccacaatgaactacaaaccattggcttccagattgaaacaattggcaaaaaggtgtcttcaaagagaattccatatgctccttctggtgaaattccgaagtttagtcttcaagatccacctaacaagaaacctaaagtgtag

SEQ ID NO: 159 - DNA, 1485 bp, synthetic construct
  source 1..1485: mol_type = other DNA
atgtttgaaataaaaaaaatttgctgtatcggtgccgggtacgttggagggccgacttgttctgtcattgctcatatgtgcccggagattcgtgtcacggttgtagatgtaaacgagagccgtatcaatgcgtggaatagccctacactgcccatctacgagccggggttaaaagaagtggtcgaaagctgtcgtgggaagaacttgttcttcagcacgaatatagatgacgctataaaagaagccgatttggtcttcatatctgtcaacactcctacgaaaacctatgggatggggaagggcagagcactggatcttaagtacatagaggcttgtgcgcgtcgtatcgttcagaattctaatggctataaaatagttactgagaagagcacagttcctgtaagggctgcagaaagtataagacgtatattcgacgcgaacacgaaacccaatttgaaccttcaagtgctttctaatccagagttcttggcagaagggacggcaataaaggatcttaaaaacccagacagagtgctaatcggcggtgacgagacccctgaaggccaacgtgctgttcaagccctgtgtgccgtttatgagcattgggtacccagagagaagattcttacgaccaacacctggtccagtgaattgagtaagctagcagctaacgcatttttagcccagcgtatctcttccatcaactccataagtgccttgtgtgaagcgactggggctgacgtggaagaagtggccacagcaatagggatggaccaacgtattggaaacaagttccttaaggcgagcgtgggtttcggaggctcctgttttcaaaaggatgtactaaatctagtttatctttgtgaagcgctaaacctacccgaggttgcccgttattggcagcaggtgattgatatgaatgactaccaaaggcgtagatttgcaagtagaatcatcgacagcctattcaatacagtcactgacaagaaaatagccatactaggcttcgcctttaagaaggatacgggtgatacaagggagtcctcaagcatttacattagtaaatatttaatggacgagggtgcccaccttcacatctatgaccccaaggttccgagggaacagatcgtagtcgaccttagccacccaggggttagtgaagacgatcaagtcagtagactggtaacaatttcaaaagatccttacgaggcgtgcgatggagctcatgcagtagtgatttgtacggagtgggatatgttcaaggaacttgattatgaaaggatacataaaaagatgctgaaacctgcgttcatatttgatggcagacgtgtacttgatggcttacataatgagttgcagaccatcgggtttcagatagaaaccatcggcaagaaggtaagcagtaagcgtatcccatacgcaccttctggcgagatacccaagttttccttacaagacccacctaataaaaagccaaaggtgtga

SEQ ID NO: 160 - protein, 428 aa, Bacillus cytotoxicus
MNICIIGSGYVGLTSAAVLADLGHHVICVDKDQNKIRSLQKGECPIYEPDLKELIHKNRNRLKYCHEIDQAIKLSSVIIITVGTPSTSNGKTDLTYFYSVIDSLVPAITSYKTIITKSTVPPGTNQYLYETLIKKGVSPSLFNVVSNPEFLREGSAVHDMFFPDRTIIGVQKNDKKSFEIMKKIYKNIHAPFFWTGLDEAEMIKYAANAFLATKISFINEIARICDVYHIDVVEVAKGIGYDFRINPHFLQAGLGYGGSCFPKDLQSLIYTATDRQVTVPILQAVQSINSTQVDLYIEKIKNCIEQSAKKITILGISFKPHTDDIRNSLAITLLEKLVPLGYDLHTYDPKAVLPIHLNKYVTQHEQLEPSIINSDCVVIATEWDEFKSLNWKQVKQAMKGDLIVDGRNCLTKEEIEKSRLRYVGVGRS

SEQ ID NO: 161 - DNA, 1287 bp, Bacillus cytotoxicus
  source 1..1287: mol_type = genomic DNA
atgaatatatgcattataggatctggttatgtggggctaacatcagcagcagtactagctgatttaggacaccatgttatttgtgtagataaggatcaaaataaaattcgttctcttcaaaaaggagagtgtcccatttacgagccagatttaaaagaacttattcacaaaaatcgtaatcgtttaaaatactgtcatgagattgatcaagctattaaactctcctcagttattatcatcaccgtaggaactccttcaacatcaaatggaaaaacagatttaacatatttctactctgttattgattcgttagttccggctataacatcatataaaacaattattacgaaaagtacagttccaccaggcacaaatcaatatttatatgaaacactcattaaaaaaggtgtttcaccatccctttttaatgttgtttctaatcctgagttccttcgcgaaggctctgctgtacatgacatgttctttccggatagaacaattataggggttcaaaaaaatgataaaaaatcatttgagatcatgaaaaaaatatataaaaatattcatgctccgttcttttggactggtttagatgaagcagaaatgattaaatatgcagcaaatgcttttttagctacaaaaatatcttttataaatgagattgctcgaatttgtgacgtataccacattgatgttgttgaagtagcgaaagggattggctatgattttcgaattaatcctcattttctgcaagctggactggggtatggtggatcatgctttccaaaagacttgcaatcacttatctatacagcaactgataggcaagtcactgtccctattttacaagcagtccaatccattaattctacacaagtagatctctatattgaaaaaatcaaaaactgtatagaacaatctgcaaaaaaaataactattttagggatttcctttaagccacatacggacgatattcgcaattcacttgcaataacacttctcgaaaaattagtgcctcttggctacgatcttcacacttatgatccaaaagccgttcttcctatccatttaaataagtacgttacccaacatgagcaattagaaccctctattataaactctgattgtgttgttattgccacggaatgggacgaatttaaatcactgaattggaaacaagtgaaacaagcgatgaaaggagacctaattgttgatggacgaaactgccttactaaagaggaaatagagaaatccagattacgctatgtaggagttggtcgctcatga

SEQ ID NO: 162 - DNA, 1287 bp, synthetic construct
  source 1..1287: mol_type = other DNA
atgaacatttgtataattggcagcggatatgttggcttaacctccgcagcagttctggccgacctgggtcaccacgtaatctgcgttgataaagaccaaaacaagataagatcactgcaaaagggcgaatgtccaatatacgagcccgacttgaaggaacttatacataagaataggaatagactgaagtattgccatgaaatagaccaagctattaaacttagctcagtgatcataattacggttggtacaccgagcacaagtaatggaaagaccgaccttacttacttctactccgtgattgacagcctagtaccagcaataacctcttacaagactattattactaagtcaactgtaccgccgggcacaaatcaatatctatacgaaacactgataaagaaaggtgtttcccccagccttttcaacgtggtaagcaacccggagttcctgagggagggtagcgcagtccatgatatgttcttcccggaccgtacgataataggggttcagaaaaacgataagaaaagcttcgagataatgaaaaagatatataagaatatacacgcgcccttcttttggacggggctggatgaagcggaaatgatcaagtatgccgcgaatgctttcctagcgaccaagattagttttattaatgagatcgctcgtatttgtgatgtttatcatattgatgtcgtagaggttgccaaaggaataggctatgactttaggataaacccgcactttttacaggccggtttaggttatggtgggtcttgcttcccgaaagaccttcaatcactgatatacacagccacggaccgtcaagtaacagtacccattttacaggctgtccagtcaattaacagtacccaagtcgacttgtatatagaaaagattaagaattgtatcgagcaaagtgcaaagaaaatcacgattctaggcatttctttcaaaccacacactgatgacattagaaattctcttgccataactttactagaaaaattggttcctttagggtacgatttacatacctatgacccgaaggctgtcctgccgatacatcttaacaagtacgtaacccagcacgagcaactagagccttccatcatcaactctgattgcgtagtgatagcaacggagtgggacgagtttaaatccctgaactggaagcaagtaaagcaagctatgaagggcgatctaatagtcgacggtcgtaactgccttacgaaggaagaaatcgaaaaatctaggctaagatatgtgggggttggaaggagctga

SEQ ID NO: 163 - protein, 432 aa, Corallococcus macrosporus
MRIAIIGTGYVGLVAGTCFAESGHDVTCVDLDARKVEALRRGELPLYEPGLEELVRRNVAARRLRFTGHLPEAVSAAQVVFIAVGTPQSERGSADLQYVLAAAEQIGRALRHYTVIVNKSTVPVGTSDQVRAVVARSTDVVFDVVSNPEFLKEGAALDDFLKPDRVVIGATSERARRLMADLYAPFVRTENPILFMDPHSAELTKYAANAMLATRISFMNDMALLCERVGANVDLVRKGMGADRRIGYAFLYPGIGYGGSCFPKDVRALMSTARDAGLELDLLRAVESTNTRQKRCLLGKALKHYGSLADHTFAVWGLAFKPKTDDMREAPSVELIEGLLGKGARVQCHDPVAGAVARQYFGDRVAYAPTCYEAAEGADGIFLVTEWNEFRRPDLKRLRALMRRPIIFDGRNVLDPRLARSEGFTYIGVGRD

SEQ ID NO: 164 - DNA, 1299 bp, Corallococcus macrosporus
  source 1..1299: mol_type = genomic DNA
atgcggattgccatcatcgggacggggtacgtggggctcgtggcggggacctgcttcgcggagtcggggcacgacgtcacctgcgtggacctggacgcgcgcaaggtggaggcgctgcggcgcggggagcttcccctctacgagccgggcctggaggagctggtgcggcgcaacgtcgccgcgcgccgcctgcgcttcaccgggcacctgccggaggcggtgtctgccgcccaggtggtgttcatcgcggtgggcacgccgcagtcggagcgggggagcgcggaccttcaatacgtgctcgccgccgcggagcagattggccgggccctgcgccactacaccgtcatcgtgaacaagagcaccgtgcccgtcgggacgtcggaccaggtgcgggccgtggtggcgcggagcaccgacgtggtgttcgacgtcgtgtccaacccggagttcctcaaggagggcgccgcgctggacgacttcctcaagccggaccgggtcgtcatcggcgcgacgtccgagcgcgcccgcaggctgatggccgacctgtacgcccccttcgtgcggacggagaaccccatcctcttcatggacccgcactcggcggagctcaccaagtacgccgccaacgccatgctggccacgcgcatctcgttcatgaacgacatggccctgctgtgcgagcgcgtgggcgccaacgtggacctggtgcgcaagggcatgggcgcggaccgccgcatcggctacgcgttcctctacccgggcatcggctacggagggagctgcttccccaaggacgtgcgggcgctgatgtccaccgcgcgcgacgcggggctggagctggacctgctgagggccgtggagtccacgaacacgcgccagaagcgctgcctgctcggcaaggccttgaagcactacggcagcctcgccgaccacaccttcgcggtgtggggcctggccttcaagccgaagacagacgacatgcgcgaggccccttccgtggagctcatcgaagggctgctgggcaagggcgcgcgcgtgcagtgccacgacccggtggccggcgccgtggcccggcagtacttcggggaccgggtggcgtacgcgcccacctgctacgaagccgccgagggcgcggacggcatcttcctcgtcaccgagtggaacgagttccgccgccccgacctcaagcgcctccgggcgttgatgcgcaggcccatcatcttcgatggccgcaacgtgctcgacccgcggctcgcccggagcgagggcttcacctacatcggcgtcggacgcgactga

SEQ ID NO: 165 - DNA, 1299 bp, synthetic construct
  source 1..1299: mol_type = other DNA
atgaggattgccattattgggaccggttacgtcggactggtcgcggggacttgcttcgcagaaagcggacatgacgttacctgcgtggacctggatgcgaggaaggtggaagctttacgtcgtggggagctaccattatatgaacctgggctagaggagctggtacgtagaaatgtggctgcaagacgtttacgtttcacaggacacttgcccgaagccgtatccgcagcacaggtggtcttcatagcagttggaacgccccagagcgagcgtggttctgctgaccttcaatatgtattagcggcggccgaacaaataggtagagcattgaggcattataccgtcatcgtgaacaagtctactgtccctgtgggtacaagcgaccaagtgagagcggttgtagcacgtagcacggacgtggtattcgacgtggtttcaaaccctgagttcctgaaagaaggcgctgcattagacgatttcctaaagccggaccgtgtagtcatcggtgcaacgagcgagcgtgcaaggcgtctaatggcggacctatacgcaccgttcgtcaggacagaaaaccctatactgtttatggacccgcatagtgctgaattgacaaagtatgccgccaatgccatgctggcgaccagaattagtttcatgaacgatatggcattgctttgcgaacgtgtcggtgccaatgttgatcttgtcagaaaaggcatgggagcggatcgtcgtatcggctacgcctttctatatccaggaataggatacggtggctcatgcttcccaaaagatgtcagggcccttatgagtacagcgagagatgctggccttgaattggacctattaagagccgtcgaaagtaccaatacgagacagaaaagatgcctgttgggcaaagctttgaaacactacggatcactggctgatcacacgttcgccgtctggggactggccttcaaaccgaaaacagatgacatgagggaggcgccatccgttgagctgatcgaaggactattaggtaaaggcgctcgtgtgcaatgtcacgatccagtggccggtgctgttgcgcgtcagtatttcggcgacagagtcgcatacgctcccacatgctatgaggcagccgaaggtgcggacggtatatttcttgtgaccgaatggaatgagttccgtaggcccgatctaaaaagactacgtgcattgatgaggaggcctattatatttgatggtcgtaacgtccttgaccctcgtttggccagatccgagggcttcacatatataggtgtaggtcgtgactaa

SEQ ID NO: 166 - protein, 427 aa, Pyrococcus furiosus
MRVSVVGSGYVGLVTGIGFVKLGNSVIFVDVDERKIQMINNAQPPIYEEGLEELMQEFRGKYYATNDYRDAILNSDITFIAVGTPSREDGSIDLTYVKEASRAIGKALREKEDYHVVVVKSTVLPGTTEEVVRPILEKYSGKKAIQDFGLAMNPEFLREGTALKDFLNPDRIVIGVQDERTKKVLEELYAPINAPKLIVDIKTAEMIKYASNAFLATKISFANEIGNICKKLGIDSWKVFEGVGLDHRISPYFFRTGIGWGGSCFPKDVRALIRKAEELGEDPIILKAVVEVNERQPLKLIELLKKHVPDLKGKTVGVLGLAFKPNTDDVRETRAYIIVRKLLEKGAHIIAYDPKAMENFKRFYPDVGEKIEYANSPEEVLEKSDVILIVTEWNEFEKVDYSGKIVIDGRRIRAAEKTARIYEGVCW

SEQ ID NO: 167 - DNA, 1284 bp, Pyrococcus furiosus
  source 1..1284: mol_type = genomic DNA
atgagagtttcggttgttggctctggttatgttgggcttgtcacagggataggttttgtcaagcttgggaatagcgttatcttcgttgatgttgatgagaggaagatccaaatgattaataatgctcaacctccaatttatgaggagggtttggaagaattaatgcaagaatttagaggtaaatattacgctactaacgattatagagatgcaattctcaattctgacattacgttcatagccgttggaactccctcaagagaagacggttcgattgaccttacatacgttaaagaagccagcagagctattggaaaggcactaagagaaaaggaagattatcatgttgtcgtagttaagagcactgtcctcccaggaactaccgaggaagttgtaagaccaatccttgaaaagtattctggtaagaaggcaatccaagactttggccttgcaatgaatccagagtttttgagagaaggtactgcattgaaggacttcctcaacccggatagaatagtcattggtgttcaagatgagaggactaagaaagtcctcgaagagctttatgctcccattaatgcaccaaagttaattgtggacattaagactgctgaaatgataaagtacgcctcaaacgctttcctagcaacaaagataagctttgcaaacgagattgggaatatttgtaaaaaactcggaatagattcttggaaagtttttgaaggagttggcctagatcataggataagcccctacttcttcaggactggaattggctggggtggctcatgctttccaaaagatgtcagagccttaattaggaaggcggaagaactgggagaggacccaataatcctaaaggcagttgttgaggtcaatgagaggcaacctctcaagcttattgaattgctaaagaagcatgttccagaccttaagggaaaaactgtcggagtgttaggattagcattcaagccaaacactgatgacgtgagagagactagggcttatattatagtcagaaaacttctggaaaaaggagctcacattatagcttacgatccaaaagccatggagaatttcaagcgcttttatccggatgttggggaaaagatagaatacgctaactcgcctgaagaagttcttgaaaaatctgatgttattttgatagttacagagtggaatgaatttgagaaggtagattactctgggaaaattgttattgacggtaggagaattagagctgccgagaaaacagcgagaatttacgagggggtgtgctggtga

SEQ ID NO: 168 - DNA, 1284 bp, synthetic construct
  source 1..1284: mol_type = other DNA
atgcgtgtatccgtcgtaggcagcggttacgtgggtctggtgacaggtattggtttcgtaaaactagggaatagtgtcatctttgtcgatgtcgatgagagaaaaattcaaatgataaacaacgcgcagccccccatctatgaggaagggctagaggagttaatgcaagagttccgtgggaagtattacgccactaacgactaccgtgatgccatacttaattccgatatcacatttatcgcggttgggacaccgagtagagaagatggctctatcgacctaacgtatgtcaaggaggcctcaagggctattggaaaggctcttcgtgagaaggaggattaccatgtagtagttgtaaaatccacagtattgccagggaccacagaagaagtggtgcgtcccatcttagagaagtattctggcaagaaagcaattcaagactttggattagcgatgaatccagaattcctgagagagggaactgcgctaaaggacttcctgaacccagaccgtatcgtgataggggtgcaagatgagcgtactaagaaggtgttagaggagctatatgcgcctatcaacgccccgaagttaatcgtcgacataaaaacggccgagatgataaagtatgcaagtaatgcttttttagcaacgaaaatatcattcgcaaatgaaattgggaacatttgcaaaaagttaggtatagattcttggaaagtctttgaaggggttggtttagaccaccgtatatccccgtacttttttaggactggtataggatggggtggatcttgtttccctaaagacgtaagagcccttatcagaaaagcggaagagctgggagaagacccgattattctgaaagctgtggttgaggtcaacgagagacagccgctgaagttgatagagttattgaagaaacacgttccggacttgaaaggcaagacagtcggagtgttggggttagcattcaagcctaataccgatgatgtccgtgaaactcgtgcctacataatcgtcagaaagctgttagaaaaaggagcccacataatcgcatatgacccgaaggctatggagaacttcaaacgtttttatcccgacgttggcgaaaagatagagtacgcgaactcaccagaggaggtgcttgagaagtctgatgtgatattaattgtcaccgagtggaacgagtttgagaaggtagattattctggcaagattgtgatagacggtaggagaattagagccgcggagaaaactgcaaggatatacgagggggtttgctggtag

SEQ ID NO: 169 - protein, 362 aa, Arabidopsis thaliana
MDPNSTVSGDGQATAAIEHRSFARIGFLGNPSDVYFGRTISLTIGNFWASVKLEPSEHLVIKPHPFHDLVQFTSLDHLLNRLQNEGYYGGVRLLMAICKVFRNYCKENDIQLHQANFSLSYDTNIPRQTGLSGSSAIVSAALNCLLDFYNVRHLIKVQVRPNIVLSAEKELGIVAGLQDRVAQVYGGLVHMDFSKEHMDKLGHGIYTPMDISLLPPLHLIYAENPSDSGKVHSMVRQRWLDGDEFIISSMKEVGSLAEEGRTALLNKDHSKLVELMNLNFDIRRRMFGDECLGAMNIEMVEVARRVGAASKFTGSGGAVVVFCPEGPSQVKLLEEECRKAGFTLQPVKIAPSCLNDSDIQTL

SEQ ID NO: 170 - DNA, 1468 bp, Arabidopsis thaliana
  source 1..1468: mol_type = genomic DNA
tgggacccaaatactgttataaaaaaggatcattaaaatggatatgttaaaaaaagaaagtaaaaaacgcgtcagggaagagaagatggatccgaattccacggtttccggcgatggtcaggcgacggcggcgatagagcatcggtccttcgctcggatcggatttctcggaaacccgagcgatgtatacttcgggcgaaccatatcattgaccatcggaaacttctgggcatccgtgaagctggagccatcggagcatctcgtaatcaagcctcatccattccatgatctcgtccagttcacctctctcgaccatctcctgaatcgtttgcaaaatgaaggatactacggtggggtaaggttgctaatggcgatatgtaaagtattccgtaactattgcaaagagaatgacattcaacttcaccaagccaacttctctctttcttatgataccaatatccctaggcagacagggctttcgggttctagtgccatcgtatccgctgcccttaactgccttctcgacttctacaatgtcaggcatttgatcaaagtacaagtccgccctaacattgttctcagtgctgagaaagaacttggcattgttgctggtcttcaggacagggttgctcaggtctatggtggtcttgttcacatggattttagcaaggagcacatggataaattggggcatgggatttacactcctatggatatcagtctcctccctcctctgcatctcatctatgctgagaatccgagcgactcagggaaggtacatagtatggttcggcaaagatggttagacggtgatgagtttataatctcatcaatgaaagaagtcggaagtctagcagaagaaggtcgaactgcattactcaacaaggaccattccaaacttgtggaactcatgaaccttaatttcgacattcggaggcggatgtttggggatgaatgcttaggagcaatgaacatagagatggtggaagtagcaaggagggttggtgcagcctcaaagttcactggaagtggaggagcagtggtggttttctgccctgaaggtccatctcaggtgaaacttctggaagaagaatgcaggaaagcgggatttacgcttcagccggtaaaaattgcgccttcatgtttgaatgattctgacattcagaccttatgaaagccttgatcaagtcttcttattcacacgatagagccattttccagagaaataaacatgtgagctttaattgggtggttgttctgtttctttttcttgcccatttttgttgtttgttccttggcttatactaatatgtttgttttgtttctttttccacattaaggtccaattttttatattatattaaaagaactaaagaacagatgatcaaaattgaagtagtaatgatatttatttgccatatttgtgctcttttttgttttgttgaaactgtatatacagaagttggca

SEQ ID NO: 171 - DNA, 1089 bp, synthetic construct
  source 1..1089: mol_type = other DNA
atggaccctaattctactgtgtcaggggacggtcaagccacggccgcgatagaacaccgttcctttgcccgtatcggtttcttaggtaacccctctgatgtgtatttcggacgtactatcagtctgacgattggtaatttctgggctagcgtcaaacttgagccaagcgaacaccttgtgataaagccacatcctttccacgacttagttcagtttacatctttggaccatttactgaataggctgcaaaatgaaggatattacgggggcgtccgtttattaatggctatctgcaaggtttttcgtaattactgtaaggagaacgatatacagcttcaccaagctaactttagtctgagctatgatacgaacatcccgagacagacggggctgagtggaagtagcgccatagtcagcgcagcacttaattgtttgctagacttctataatgtgagacacttgatcaaagtgcaggttaggcccaatatcgtacttagcgcagaaaaggaactggggatcgtagcagggctacaggacagagtggcacaggtctatgggggtttagttcatatggatttttcaaaggaacacatggacaaattagggcatggcatatacactccgatggacatctcactgctgcccccattgcatttaatctacgcggaaaatcccagcgactccgggaaagtgcacagcatggtgagacaaagatggttagacggtgacgaatttataatttcaagtatgaaagaggtagggagtttagcggaggaaggcagaaccgcgctattgaataaagaccatagtaagctggtcgagttaatgaatttgaactttgacatacgtaggcgtatgtttggtgacgagtgtctaggtgcaatgaacattgaaatggttgaagtagcaagacgtgtcggtgccgcctctaaattcactgggagcggcggggccgtagtggttttttgccctgaaggaccgtctcaggtcaaactgttggaggaggaatgtaggaaggccggttttaccctacagcctgtcaaaatagccccaagttgcttgaatgacagtgacatccaaacattatag

SEQ ID NO: 172 - protein, 313 aa, Thermothelomyces thermophilus
MAAVDIRPAQRDGPALEAITDAVAMVNILRGMATSETTAFDAESRFDSDKDKSAFRKYDEACDRVKGFYAEQHAKQTVAHNLAARQRFYSPTRKRPEMTVWEAIEKLNALIDNSDPDTELSQIHHLLQSAEAIRRDGKPRWMQLVGLIHDLGKLMLFFDGCAEGQWEVVGDTFPVGCQFDERCIYPETFKANPDSSHEVYGTRYGIYQPGCGINNLMMCWGHDEYLYLVIKDQSTLPPEALAMVRFHSFYPWHREGAYMDFMAEGDEALLHAVRAFNPYDLYSKSDEVPKVEELKPYYLELIDEFFPQKVIKW

SEQ ID NO: 173 - DNA, 942 bp, Thermothelomyces thermophilus
  source 1..942: mol_type = genomic DNA
atggctgcggttgatattcgcccagctcagcgggacgggccggcgctcgaggccatcacggacgccgtggccatggtcaacatcctccgagggatggcgacctcagagacgacggcattcgacgccgagtcgcgcttcgacagcgacaaggacaagtcggcattccggaagtacgacgaagcctgcgaccgggtcaagggcttctacgccgagcagcacgccaagcagacggtggcccacaacctggcagcccgccagcgcttctactcgcccacgcgcaagaggcccgagatgacggtctgggaggccatcgagaagctcaacgcactgatcgacaacagcgacccggacacggagctcagccagatccaccacctgctccagagcgccgaggcgattcggcgcgacggcaagccccggtggatgcagctggtcggcctgatccacgacctgggcaagctgatgctcttcttcgatggctgcgccgagggccagtgggaagtcgtcggcgacaccttccccgtcggctgccagtttgacgagcggtgcatctacccggaaacattcaaggccaaccccgactcgagccacgaggtctacggcacacgctatggcatctatcagccgggctgcggcatcaacaatctcatgatgtgctggggccacgacgagtacctgtacctggtaatcaaggatcagagcaccctaccccccgaggctctcgccatggtgcgcttccactccttctacccctggcacagggagggagcctacatggacttcatggctgagggcgacgaagcgctgctgcatgccgttcgcgctttcaatccctacgacctctacagcaagtccgacgaagtaccaaaagttgaggagttgaagccatactatcttgagcttatcgatgaattcttccctcagaaggttattaagtggtga

SEQ ID NO: 174 - DNA, 942 bp, synthetic construct
  source 1..942: mol_type = other DNA
atggctgcagttgacattcgtccggcacaaagagatggaccagcccttgaggcaatcacggatgcagtagctatggtgaatatactgcgtggtatggctacctccgagacgactgctttcgatgcggagagcaggtttgatagtgacaaggacaagagcgcttttcgtaaatatgacgaggcgtgcgacagggtgaaaggattctacgccgagcaacatgcgaagcagacagtggcacataatctagccgcgaggcaaagattctattcccccaccaggaaacgtccggagatgacggtctgggaggctattgaaaaacttaatgcactgatagacaactccgatcccgacactgaactatcacaaatacaccacctgttgcagagcgcggaggctatcagaagggacggaaaacccaggtggatgcagctagttgggctgatccacgatctgggcaaattaatgttattttttgatggatgtgcggaaggccaatgggaggtagtgggggatacattccccgtaggatgccagttcgatgaaagatgtatatacccagagacctttaaagcaaatccagattcatctcacgaggtctacggtacacgttatggtatataccagcccggctgtggcattaataatctgatgatgtgctgggggcatgatgagtatttgtatctagtcatcaaagaccaatcaacgcttccaccggaagcattggcgatggtgcgtttccacagcttctatccttggcatagggagggggcttatatggactttatggccgagggtgacgaagcgttactacacgcggttagggcctttaatccctatgacctatattccaagtcagatgaggtgccgaaggtagaggaactaaaaccctattaccttgaacttatcgacgaattcttccctcaaaaggtcatcaaatggtag

SEQ ID NO: 175 - protein, 298 aa, Quillaja saponaria
MAEATQRYAVVTGSNKGIGFGICKQLASKGIKVVLTARDEKRGLEAVEKLKEISLAGHVVFHQLDVSDPASVTSLEDFIKTQFGKLDILVNNAGITGTTVDADALAASGFGTGGERKPIDWSKLVIQTYESVEKAFNTNYYGGKRMTEALIPLLQLSDSPRIVNVSSAMGQLENIPSGWAKEVLTDVDNLTEGKLDEVSTQFLKDFKEGSLETKGWPSLMSSYIVSKAVLNAYTRILAKKYPAFCINCVDPGYVKTDINHHTGQLSVDEGAESPVRLALLPNGGPSGVFFSRTEEAPF

SEQ ID NO: 176 - DNA, 897 bp, Quillaja saponaria
  source 1..897: mol_type = genomic DNA
atggcagaagcaacgcagaggtatgctgttgtgacaggatctaataagggaattggatttgggatatgcaagcagttggcttctaagggaatcaaagtagtgttaacagctagagatgagaagagaggtcttgaagcagttgagaaattgaaagaaattagtctggctggtcatgtggtttttcatcaactcgatgtgtctgatcctgctagtgttactagccttgaagatttcatcaaaacccagtttgggaagctagatattctggtaaacaatgctgggataacaggaacaactgtagatgctgatgctttagcagcttcaggcttcggtacagggggtgaacgtaagcctattgattggagtaagttagtgatacagacttatgaatcagttgaaaaagctttcaacacaaactattacggtggcaaaagaatgacagaagcacttatacccctcctccagctatcagactcacccaggattgttaatgtttcctctgctatgggacagttagagaatatacctagtggatgggcaaaggaagtgctcacagatgttgataacctaacagaaggaaaattggatgaggtttcaacccagtttttgaaagatttcaaagagggttcattggaaaccaaaggctggcctagtcttatgtcttcttatatagtctcaaaagctgttttaaatgcctacacaaggattcttgctaagaaatacccagctttctgcatcaattgtgtagatcctggctatgtgaagacagacataaaccatcatactggccaattaagtgttgatgaaggtgctgaaagtcctgtaagactggccttgctgcctaatggtggtccttctggcgtgttcttctccaggacagaagaagcaccattttga

SEQ ID NO: 177 - DNA, 897 bp, synthetic construct
  source 1..897: mol_type = other DNA
atggcagaagcgactcagaggtatgcagtagtgactgggagtaataagggcataggatttgggatatgtaagcagcttgcgagtaaagggataaaggtggttttgaccgcaagggacgagaagaggggacttgaggctgttgagaaacttaaagaaatttccttggcggggcatgttgtcttccaccagcttgatgtttcagaccccgcctcagttacgtccttagaagatttcattaagacccaattcggaaaattggacattttggttaacaacgccggtatcactggaacgaccgtagacgccgacgcattagcagccagtggtttcgggacaggaggtgagaggaaacctattgactggtctaaactagtgatacaaacctacgaaagcgtcgagaaggctttcaatactaattattacggtggtaagagaatgaccgaagcactaatcccgctacttcaactatcagattccccgcgtattgttaacgtaagttcagcgatggggcaactggaaaatataccttctgggtgggccaaggaggttttgacagatgtggataatctaaccgaaggcaaattggatgaggttagcacccagttcctaaaagatttcaaggaaggaagtctagagactaagggttggccgagcctaatgagctcttatatagtcagcaaagcagttctgaatgcgtacaccaggattctagcgaagaaataccccgcgttctgtatcaattgcgtggatcccggttacgttaagacggatattaaccatcacactggtcaattaagtgtagatgaaggggcagagtctccagttagattagcgttactaccgaacggagggccttcaggagtattcttcagcaggacggaagaggcacctttctga

SEQ ID NO: 178 - protein, 543 aa, Quillaja saponaria
MEDLKPSSANSCPLTTLGFLDRAATVYGDCTSIIYNSTSYTWSTTHRRCLQVASSLASLGIKPGHVISVLAPNIPAMYELHFAVPMTGAILNTINLRLDARNISVILRHAESDLIFVDCLSRATVLEALSLFPPHVPRPRLILITDDAVDSSSPTVDFIDTYDGLIEKGDPEFKWVRPSSEWDPMTLNYTSGTTSSPKGVVHCHRGVFMMTIDSLIDWAVPKQPVYLWALPMFHANGWTFPWGIAAMGGTNICVRKFNAELIYRLIEQYDVSHMCGAPVVLNMLSNSPEQKPLKNPVQILTAGAPPPATVLFRTESLGFVVSHGYGLTETAGLVVSCAWKQQWNRLPATERARLKARQGVRTIGMTAIDVVEPESGISVKRDGSSLGEIVLRGGSIMLGYLKDLKGTSKSMKNGWFHTGDVGVMHPDGYIEIKDRLKDVIISGGENLSSVEVESMLYTHPDVIEAAVVARPDEFWGETPCAFVSLKGGLDVKPTEKEIIEYCRSKLPHFMAPKTVVFQEELPKTSTGKIQKFLLREIARGKGP

SEQ ID NO: 179 - DNA, 1632 bp, Quillaja saponaria
  source 1..1632: mol_type = genomic DNA
atggaagatcttaaaccaagctctgcaaattcatgccctttaaccacgcttggatttttggatagagcagccaccgtttacggtgattgcacctctatcatttacaatagtacctcatacacgtggtcaaccacccaccgtcgatgtctccaggttgcttcatctctcgcatcactcggcatcaagccaggccatgtcatctccgttttagcccccaatattcctgctatgtacgagcttcactttgctgttccaatgactggtgctatacttaacaccatcaatctccgtcttgacgcccgtaacatctccgtcatactccgtcacgctgagtcagatctcatctttgtcgattgtctttcacgggctactgttcttgaagctttatctttatttccaccgcatgttccccgtccccggctcatccttattacggatgatgcggtcgattcttcatcacctaccgttgattttattgatacttatgatggactgattgagaaaggggatcctgaattcaagtgggtccgacctagtagtgagtgggacccaatgactttgaattacacttctggtactacatcatcgccaaagggtgtggttcattgccatagaggggttttcatgatgacaattgattctttgattgactgggccgtacctaagcagccggtatatttgtgggccctacctatgttccatgctaatggatggaccttcccctgggggattgctgccatgggagggaccaatatctgtgtacgaaaattcaacgcggaattgatttaccgtttgatcgagcaatatgatgtgagccacatgtgtggtgcacctgtggtactcaacatgctatcaaattcgcctgaacagaaaccgctaaagaacccggttcagattctcactgctggagctccaccacctgcaacggttctcttccgaactgaatcactgggtttcgttgtgagtcatgggtacgggttgactgaaacagctgggttagtcgtttcctgtgcttggaagcaacaatggaatcgtttaccggcgaccgagagagctcgattgaaagcaaggcaaggagtgaggacaattgggatgacggcgatagacgtggtggaacccgaatcgggtatcagtgtgaaacgggacgggtcgtcactcggtgaaattgtgctacgaggcgggtcgattatgttgggttatctgaaagatctaaagggtacctccaaatccatgaaaaatggttggttccatactggagacgtgggtgtgatgcacccagatgggtacatagaaatcaaagatcgattgaaggatgtaattatcagcggtggagagaatctgagcagcgtggaggtggaatcaatgctgtacacccacccggatgttatcgaagcggcagtagtggcccgacccgatgagttctggggtgagacgccatgtgcatttgtgagcttaaaaggtggattggatgtgaaaccaacagagaaagagataattgaatattgtagatcaaagttgccccactttatggcgcccaaaactgtggtttttcaggaggagcttccaaaaacatccaccgggaaaattcagaagtttctgctcagagagattgccagaggtaagggcccataa

SEQ ID NO: 180 - DNA, 1632 bp, synthetic construct
  source 1..1632: mol_type = other DNA
atggaagacttaaagcccagttcagccaacagttgcccgctaactaccttaggattcctagacagggcggcgacagtatacggggattgtacttcaattatatacaacagtacatcctatacttggtccaccacccacagacgttgcttgcaagtggcgtccagccttgccagccttgggatcaagcctggccatgtcatatcagtactagctccgaacatacctgcgatgtatgaactacatttcgctgtgccgatgacaggcgcgattttgaacaccataaacttacgtctggatgctagaaacatttcagttatattgaggcatgcggagtcagacttaatcttcgtagattgcttgtctcgtgccacagtcttagaagcactttctttatttccgccacatgtcccgcgtcccagattgatcctaatcaccgacgatgcagtggacagttcatctccaacggtcgatttcatcgacacatacgacggtcttatagaaaaaggggatccggagttcaagtgggttaggccgagcagtgaatgggacccgatgacgctgaactatacgagcggtactacgagctcaccaaaaggtgttgttcactgtcatagaggtgtattcatgatgacgatagactcacttatagattgggcggtgcccaagcaaccagtatacctatgggcgctaccaatgttccacgcaaacggatggacgtttccttggggcatagcggcaatggggggaacaaatatttgcgtcaggaagtttaatgcggaacttatctacaggcttatcgagcaatacgatgtttctcatatgtgtggtgctccggttgttttaaacatgctatccaactcaccagaacagaaaccattaaaaaatcccgtacagatcctgacagcaggtgcaccaccgccagcgactgttcttttcagaaccgagtctttaggatttgtggtaagtcacgggtacggtcttaccgaaacggccggtttggtcgtgtcttgcgcttggaagcagcaatggaatagactaccggccaccgaaagagcacgtctgaaagcccgtcaaggagttagaacaataggtatgaccgccatcgacgtggtcgaaccggagagcggtatatccgtgaaaagagatggtagctccttaggtgaaattgttttaagggggggttcaataatgttgggatacttgaaggacctaaagggtacatcaaagtctatgaaaaatgggtggtttcataccggcgatgtcggagtgatgcacccggatgggtatatagaaataaaagatagattaaaagacgtgataatcagcggtggggaaaatttaagctcagtcgaagttgaatctatgctttatacacacccagacgtaattgaggccgccgttgttgcccgtccggacgagttttggggagaaacgccttgtgcctttgtaagcttgaaaggtgggctggacgttaaacccacggagaaagagatcattgagtactgtagatccaagctgcctcatttcatggccccaaagacagtagtctttcaagaagaactaccaaaaacttccacggggaaaatacaaaagtttcttctgagggagattgcgagaggtaaaggcccttaa

SEQ ID NO: 181 - protein, 400 aa, Quillaja saponaria
MAPVTNTNGVQSQLATNRGVAAILAMGTATPSNRIIQSDYPDFYFTVTGSDHMTDLKNKFRRICERSTIEKRYLHLTEEIHKAHPNISDPKAPSFDVRQEILSEEVPKLGKEAALKAIKEWGQPESKITHLIFYTTSCLNLPGGDLYLCELLGLDPSVNRLMLYYQGCHAGGTALRVAKDIAENNAGSRVLVVCAELSTVVTFQGPCATQIDSLVMQALFGDGAVALIIGANPNTSIERPLFQLVLASQTTIPKSEYAIQGYLREKGALYYMNKDIPVLISNNVKKVVTDAMMNSVGLTMGENDWNSLFYLVHPGGPSILDAVEKQLGLEKEKLRASRHVLREFGNMWSTTVLFAMDEMRRKSVVEGKSTTGEGLELGVLLGFGPGLTVETVVLRSVPVV

SEQ ID NO: 182 - DNA, 1203 bp, Quillaja saponaria
  source 1..1203: mol_type = genomic DNA
atggctccggtgaccaacactaacggagttcagtcccagttggcaactaataggggtgtagcagcaatactagccatgggtacagcaactccatccaatcgtatcattcagtctgattaccctgatttttatttcacagtcaccggaagcgaccacatgaccgatttgaaaaacaagttcaggagaatttgtgagagatcaacgattgaaaaacgttacctacatctcactgaggaaatccacaaggcgcaccctaacatctctgaccccaaagctccttcctttgatgtccgtcaagaaatactttctgaggaagtaccaaaacttggcaaggaggcagctttgaaggccatcaaagaatggggccaacccgaatcaaaaatcactcaccttatattctatacaacttcttgcttaaacttgcccggcggtgacctttatctttgtgaacttcttggtctcgacccctcagtcaacaggctcatgttatattaccaaggctgccatgccggagggacagccctccgtgttgccaaggacatagccgagaacaatgccggttcacgtgtccttgttgtttgcgcagaattgagtacagtggtaacgtttcaaggtccgtgtgctacccagattgatagtctcgtaatgcaagccctgtttggtgatggtgcagttgccctgatcattggtgcaaatcctaatacctcaatcgaacgtccattgtttcaactagtgttggcttcacaaacaactatccctaaatctgagtatgcaattcaaggatatttgcgcgaaaagggtgccctttattatatgaataaagacatccctgttttgatatctaacaatgtcaagaaggttgtaacagatgcaatgatgaattcagttggcttgacaatgggtgagaatgattggaactccttgttttacctagttcatccgggtggaccgagtattctcgacgcggttgagaagcaacttggattggagaaggagaaactaagggcaagtaggcatgtgttgagggagtttggaaacatgtggagtaccactgtgttatttgcaatggatgaaatgaggaggaaatcggtggtggaagggaagagcaccaccggtgaagggttggagttaggtgttttattaggatttgggccaggtttaacggtggagacagttgtattgcgcagtgtcccagttgtgtaa

SEQ ID NO: 183 - DNA, 1203 bp, synthetic construct
  source 1..1203: mol_type = other DNA
atggccccagtgacaaatacaaatggggtacaatctcaattagccactaaccgtggtgtcgcagctattttggcaatgggaactgccaccccctcaaaccgtattattcagtcagactaccctgacttctatttcacggtaacagggtccgaccatatgacggatttgaaaaataagttcaggagaatttgtgagagatcaactattgagaaaaggtatttgcaccttacagaggaaatacataaagctcatccgaacattagcgacccgaaggcgcccagctttgatgttagacaggagatactatcagaagaagttccgaagctgggaaaggaagcggctctaaaagctattaaggaatggggacaaccagagtccaagataacccacctaattttttacacgacaagttgcttgaaccttcctggtggcgatctttatctttgtgagttgttaggcttggacccttcagttaaccgtctaatgttatactaccaaggttgtcatgctggcggaacggctcttagggttgccaaggacatagctgagaacaacgcaggctctcgtgtattggtagtgtgcgctgaactgtcaacggtggtaaccttccaaggtccgtgtgcaacccaaattgactcactggtcatgcaggccctgttcggcgacggcgcagtggctttaattatcggagccaacccgaacacaagtatagaaaggccactgtttcagctagtcctagcatctcaaacgaccattcctaaatctgagtatgctatacaaggatatctaagagaaaaaggggcactatactacatgaataaggacatacctgttctgataagtaataatgttaagaaggtagtaaccgacgccatgatgaatagtgtaggcctaactatgggcgagaacgactggaactctttattctacctagttcatccaggaggaccatcaatactggacgcagtagaaaaacaattggggttagagaaggagaagcttcgtgcgagtaggcacgttctaagagaatttggtaatatgtggtctacaaccgtcttgtttgcaatggatgagatgcgtaggaaatctgtagtggagggtaagtcaacaaccggcgaaggactagagttaggcgttcttctaggattcgggcctggcctgacggtcgaaacggttgtgttgaggtcagttcctgtcgtctaa

SEQ ID NO: 184 - protein, 398 aa, Quillaja saponaria
MGSLDQDFQPNQVPTIRADAATVLAIGTANPPNCIPQAEFADFYFRVTNSNHMTDLKKKFKRICEKSSIKKRYVHLTEKILNENPNICTFKEPSLTARQNIATEEIPKLGKEAALKAIEEWGRPKSEITHLVFCTIGGAEMPSADYQLVHLLGLNPSVKRFMLYYQGCFAGGSVLRLAKDLAENNVGARVLVVCAEVSIPYFHGPSEAHFDSNLIWQMLCGDGAGAAIVGSNPRKLIERPLFQLVSASQTIVPNSEGAIKGTLSEMGIIFNLVDKVPVLIGNNIENCLEEAFGSLGISNWNSLFWVAHPGGPAILNQIEEKLKLKEEKLKASRHVLSEYGNMSSACVFFILDEMRRRSKEEGKITTGDGLEWGVLFGFGPGLTVETIVLRSISTNEAH

SEQ ID NO: 185 - DNA, 1197 bp, Quillaja saponaria
  source 1..1197: mol_type = genomic DNA
atgggatccttggatcaagattttcaaccaaaccaggtaccaacgattcgtgcggatgcagccacagtactagccattggaacggcaaacccacccaattgtattccccaagctgagttcgctgatttctatttccgagtaaccaacagcaaccacatgactgacctcaagaagaaattcaagcgcatctgtgaaaaatcatccataaagaaacgttacgtgcatctgacagagaaaattctgaatgaaaatcccaacatatgtaccttcaaagagccatctcttacagcacgtcaaaacatagcaaccgaggaaataccaaagcttggtaaagaagcagcactcaaagccattgaagaatggggaagacctaaatcggaaatcacccaccttgtattttgcactattggtggtgctgaaatgcctagtgcagattatcaacttgttcatcttttaggtcttaacccatctgtgaagaggttcatgctttactaccaaggttgctttgcaggtggctctgtactccgtttagcaaaagatcttgctgagaacaatgttggggctcgtgttcttgtagtgtgtgctgaggtttcaatcccttattttcatgggccttctgaagctcattttgattccaatttgatatggcaaatgttgtgtggggatggtgcaggagctgctatcgttggatcaaatccacgtaaattgatcgaacggccgttgtttcaacttgtgtctgcttcccaaactatagtacctaattcagagggtgcaattaagggaactttgagtgaaatgggtattatttttaatttggttgataaggtaccagtgttgataggaaacaacatcgagaattgtttggaggaagcatttgggtcactgggtattagtaattggaattcattgttttgggtagcccatccaggtggcccagccattttgaaccaaatcgaagagaaactgaaactcaaagaagagaaattaaaagcaagtagacatgtattaagcgaatacgggaatatgtcgagtgcttgtgtgttttttatattggacgagatgagaaggaggtctaaggaggaaggaaaaatcactactggtgatggattggagtggggggttttatttggatttgggcctgggttaactgtggaaaccattgtgctgcgtagcatttctacaaatgaggcccattaa

SEQ ID NO: 186 - DNA, 1197 bp, synthetic construct
  source 1..1197: mol_type = other DNA
atgggttcacttgaccaggattttcagccgaaccaagtgcccacaatccgtgcggatgcagcgaccgtcttggctattgggacggcaaatccacctaactgcatcccccaagcagagtttgcggatttttatttcagagtgacgaatagtaatcacatgactgacttgaagaaaaagttcaaaagaatctgcgagaaatcatctatcaagaagaggtatgtgcatcttacagagaaaatcttaaacgagaacccaaatatatgcacgttcaaggagccgagcttgaccgcaaggcagaacatcgccactgaagagatacccaaacttgggaaggaggcggcgttgaaagccatcgaggagtggggaagaccgaagagtgagattacccacctagtgttttgcactatcgggggtgcggagatgccctctgcggattaccaactggtgcacctgctagggctgaatccttccgttaagagattcatgttatactaccagggatgtttcgcaggaggctctgtgttgcgtttagcaaaggacctagccgagaacaacgtgggtgcaagggtattagtagtatgcgccgaagtctctataccgtactttcatggaccctcagaagctcactttgattctaaccttatctggcagatgttatgcggcgatggcgctggtgcggctatagtcggttccaatccgcgtaaactaatagaaagacctctatttcagttggtctcagcgagccagaccatagtaccgaactcagaaggcgcaattaagggaacgcttagcgagatgggaattatttttaaccttgtcgacaaggtccccgtcctaatcgggaataacatcgagaactgtctggaagaagcgtttgggtcattgggcattagtaattggaacagccttttctgggtggctcacccgggggggccagcgatactgaatcaaatcgaagagaaattaaagttgaaagaggagaaactgaaagctagccgtcatgtgttgtccgagtatgggaacatgtcttccgcttgcgtcttttttatcttggatgagatgaggaggagaagtaaagaagagggtaaaataaccacgggtgacggactggagtggggggtcttgttcggatttgggcccggattaaccgtggaaacgatagtcctgaggagtatctccactaacgaggctcactaa

SEQ ID NO: 187 - protein, 323 aa, Quillaja saponaria
MAAAESERVCVTGAGGFVASWLVKHLLSKGYLVHGTIREPGNHKYAHLEKLEKASEKLKLFKADVLNYDSLYSAIEGCSGVFHVASPAPAGPVSNPEVELFEPAVKGTLNVLKACVEAKVKRVVVVSSISAVVLNPKWPKEKVMDESCWSDTEYCRNTQNWYYVSKTEAEWEALEFAKTNGIDVVTVCPALVFGPLLQSTVNASSFGLAMLMKGFQSTMENKNLDIVDVRDLADALLLVYEKREAEGRYICLAHSVKTQEMVDKLRTLYPNYGYPSKIVDVEEDTRLSSDKLHKLGWTTFRPLEETFTDSVESYRKAGVLETN

SEQ ID NO: 188 - DNA, 972 bp, Quillaja saponaria
  source 1..972: mol_type = genomic DNA
atggcggcggccgaaagtgaaagggtatgtgtaacgggggctggaggatttgtggcctcttggcttgttaagcatctcctctccaagggatatttagtccatggaaccatcagagaacccggtaatcacaagtatgctcatttggagaaactggagaaagcttccgagaagcttaaactcttcaaggcagatgtcttaaactacgattctctttactcagcaatagaaggatgctctggagttttccatgttgctagtcccgctcctgcagggcctgtatcaaatcctgaggtagagctgttcgagcctgctgtgaaaggaacacttaatgtacttaaagcttgtgttgaagcaaaagtaaagcgagttgtggttgtatcatctatatctgctgttgtgctgaaccccaagtggccaaaggaaaaagtcatggatgagtcctgctggtctgatacagaatactgcagaaacactcagaactggtattatgtttccaaaacagaagcagagtgggaggctctggaatttgcaaaaacaaatgggattgatgttgtaacagtctgtccggcccttgtctttgggccactgcttcaatccactgtgaatgctagtagttttggtctcgccatgcttatgaaagggttccagtcgacaatggagaacaagaatttggatatagtagacgtgcgggatttggctgatgcattgcttttagtgtatgagaagcgcgaggctgaaggaagatacatatgtctagcacactctgtaaagacgcaggaaatggtcgataaacttaggactctgtatcccaactatggctaccccagcaagattgttgatgtagaagaagacacgaggttgagctccgacaaactgcacaagttgggctggaccacattcaggccattggaggaaacttttactgattctgttgaaagctatcgaaaggcaggagttctcgaaactaactag

SEQ ID NO: 189 - DNA, 972 bp, synthetic construct
  source 1..972: mol_type = other DNA
atggctgcggctgagagcgaaagagtctgtgtaacgggagctgggggttttgtggcatcttggctagtcaagcatttactgtccaagggctatttagtccacggaacgataagggaacctggtaatcataaatatgcacatctggagaagttagagaaagcgagtgaaaagctgaagttattcaaggctgacgtgctaaactacgactctctttactccgctatcgagggatgctccggggtgtttcacgtcgctagtccggcaccggcgggtcccgtatctaatcctgaggttgagcttttcgagccagcggtgaagggaacattgaacgttctgaaagcgtgtgtggaagcgaaggttaagagagtggtggtggtttcttccatatccgctgttgtcctgaatccaaagtggcctaaggaaaaagtcatggacgaatcatgctggtccgatacggaatattgtcgtaacactcagaactggtactacgtaagtaagaccgaggcggaatgggaagcattggagtttgccaagacgaacgggatagacgttgttactgtgtgtccggcattagtgtttgggccactactacagtccactgtaaacgcgtcctcctttggattggcaatgttgatgaaaggtttccaatctaccatggagaacaaaaacctagacattgttgacgttagagacctggcagacgctttgctattggtatacgagaagagggaagcagaaggccgttacatctgtctggcacactcagttaaaacacaggaaatggtggataaacttagaaccctttacccgaactacgggtacccgtcaaagatagttgacgtagaagaggatacgagactatcctcagacaagttgcacaaactagggtggacaacctttaggccgctggaggaaacattcaccgattctgtcgagtcataccgtaaagcgggcgtcctagagactaattag

SEQ ID NO: 190 - protein, 320 aa, Quillaja saponaria
MECCIVARLKTQPLWFALLFTLGVLSLLKLSFAFLKWVYVNFLRPPKNLKKYGSWAVVSGPTDGIGKGFAFQLARKGLNLVLVGRNPDKLKDVSDSILAKFEKTQIKTVVVDFTGDLSEGIVRIREAIEGLDVGILINNVGVSYPYARFFHEVDERLLKNLIKVNVEGTTKVTQAVLSGMLKKKKGAIVNIGSGDANILPCDPLYAVYAATKSYIDQFSRCLYVEYKGSGIDVQCQVPMYVATKMASIRNSSFLVPSIDGYAKAGLRWIGYEPLCTPYWPHSLIWALADSLPESVVHAWRLKFNLAIRKRGQLKDARKKE

SEQ ID NO: 191 - DNA, 963 bp, Quillaja saponaria
  source 1..963: mol_type = genomic DNA
atggaatgctgtatagtagctaggctaaaaacccaacctttatggtttgctttgctttttactttgggtgttctctcacttttgaaattatcttttgcttttctcaaatgggtctatgttaattttcttaggcctcccaaaaacctcaagaaatacgggtcgtgggcagttgtttctggaccaactgatggtattggaaagggttttgcttttcaattggctcgtaaagggcttaatctggtcttagttggtcgaaatccagataagctgaaagatgtttctgattccattttagctaaatttgagaaaacccagattaagactgttgttgttgattttactggtgatttatccgagggtattgttaggattcgtgaagccattgaaggattggatgttgggattctaattaacaatgtcggagtttcctatccttatgctaggttcttccacgaggtagatgagcgactcttgaagaatttgattaaagttaatgttgaaggcactacgaaggttacacaagctgttctgtctgggatgctcaagaagaagaaaggagctattgtgaatattggctctggggacgcaaatatacttccttgtgatcctctttatgctgtttatgcagccacaaaatcgtatatcgaccaattctcgaggtgtctctatgttgagtacaagggcagtgggattgacgtccagtgccaggttcctatgtatgtggctacaaagatggcatcaattaggaattcttcctttcttgttccttcaatagatggctatgccaaagcaggtttgcggtggataggctatgagcctctatgcacaccttactggcctcattctcttatttgggctttggcagactcattgccagagtctgtcgtccatgcatggcgcttgaagttcaaccttgcaattcgaaagagggggcagcttaaagatgcaaggaagaaagaataa

SEQ ID NO: 192 - DNA, 963 bp, synthetic construct
  source 1..963: mol_type = other DNA
atggagtgctgtatagtagctagattgaagacacaaccgctatggtttgcgctgctatttacgcttggcgtcttatccttgctaaagttgtccttcgcctttctaaaatgggtttacgtcaactttttaaggccccccaagaacctgaagaaatatggttcatgggccgtcgttagcggtcctaccgatgggattggcaaagggttcgcattccagttggctaggaagggattaaaccttgtactagtaggacgtaatcccgataagttgaaggatgtctccgactctattttagctaagtttgaaaagacccagatcaaaacggtagtagttgattttactggcgacttgagtgaagggatcgttagaatcagggaagcaatagaaggcctagatgttgggatactaattaacaatgttggtgtgagctacccgtacgctagattctttcacgaagtggatgagagattgctaaaaaacttaataaaggttaatgtggaaggaacgactaaggtcactcaagcggttttgtctggtatgttgaaaaagaaaaagggtgctatcgtgaatataggaagtggggacgccaatatccttccgtgcgacccactttatgccgtttacgcagcgactaagtcctatatagatcagttttcacgttgcttatatgtagagtataagggaagtggtatcgatgtccaatgtcaggttccgatgtacgtggccacaaaaatggccagcataaggaacagtagcttccttgtcccatcaatagatggttatgcgaaagcagggctgaggtggattgggtacgaacctctatgtactccatattggccacattcattgatctgggctcttgccgactctttacccgagagcgtggttcacgcgtggcgtttgaagtttaatcttgccataagaaagagaggtcaacttaaggacgcgcgtaaaaaggagtaa

SEQ ID NO: 193 - protein, 466 aa, Quillaja saponaria
MMEVHTTSENCIKPSQPTPSHLQNLKLSNHHSQAPDIRTNLTFFFSSNFNNPVQPGDHDATTNFTLQSKLVQNSLATTLTILYPFAGRFRNDDTIICKDDGAFFIEAKTDTKLSDFLAQPDLPLAIMDKLVPVATDAKYNGSLLILKFTLFGCGGSAVTISITHKISDLATILTLLNCWTALSRGGDGGGSSPFIQPDLNFIGRPVPSTSEVPPPSSGKNFIPPNSKYVTKRFIFSAAKIKELKARVINKIRKEEDNVFPSRVDVVLALIWKCALASVNSGSRSGNAQTFRPSVMMQAVNLRNRTDPPLPESSIGNLAILLPVWVEKEEDTELHELVSRLLTVKVRANRLKKKYQGYEDPEQVIISMESDSVKEIIEVRKKLKDFSTYVAASVVNAPLYDVDFGWGKPAWVTSTPNTVMANSIYLLDTKDAGGIEVLMNMLKEEDMIVFESNQELLQSAMVNPTII

SEQ ID NO: 194 - DNA, 1401 bp, Quillaja saponaria
  source 1..1401: mol_type = genomic DNA
atgatggaggtacataccacatcggaaaattgcattaagccctcacaacccactccttcccaccttcaaaatttgaaactctctaatcatcatagccaagcacccgatatccgtaccaacctcaccttcttcttctcctctaattttaacaatccagtccagcctggtgaccatgacgctactaccaattttacactccaatccaaacttgttcagaattcattggctacaactctcacaattctctacccttttgctggtcgattccgaaacgacgacaccatcatttgcaaagacgatggcgccttctttattgaagcaaaaaccgacaccaaactttctgactttcttgcccagccggacttgcctctagctataatggacaaattagtccccgtagctaccgacgccaagtataatggttctctgctaatcctgaaatttactttgtttggctgcggtggctcagccgtaaccatctcaataactcacaagatttccgatctagcaaccattttgacactgcttaattgctggactgctttatctcgtggtggtgatggtggtggttccagtccgtttattcagcctgatttgaatttcattggccgccctgttccaagtacgagtgaggttcctcctccatcttctggaaagaattttattcctccaaatagcaagtacgttaccaaaaggttcatattcagtgcagcaaagattaaagaactgaaagccagagtcatcaacaagattcgtaaagaagaagacaatgttttcccttcccgtgtggatgtagtgctggcactcatttggaaatgcgctttggcttctgttaattctggttccagatctggaaatgcacaaacatttaggccgtcggtaatgatgcaagccgtgaacctcagaaaccgtacagatccaccattaccggaatcttcgattgggaacttggcaatactattaccggtgtgggtggagaaagaggaagacacggaattacatgaacttgttagcagattgttaacagtgaaagtgcgtgctaacagattgaagaagaaataccaaggttatgaagatccagagcaagttattatttcaatggaatctgattcagtgaaggaaataatagaggttcggaagaaattgaaggatttcagtacttatgttgcggcgagtgtggtgaatgctccattgtatgacgttgattttgggtggggaaaaccagcatgggtgaccagcacgcccaacacagtaatggcgaattctatatatttgttggataccaaagacgccggtgggattgaagttttgatgaatatgttgaaagaggaagacatgattgtctttgaaagcaatcaggagttgcttcagtctgccatggttaatcccactatcatttga

SEQ ID NO: 195 - DNA, 1401 bp, synthetic construct
  source 1..1401: mol_type = other DNA
atgatggaggtccacactacctctgaaaactgcattaagccatctcaacccacacctagtcacttgcaaaacctaaaattaagcaatcatcatagtcaggcacccgatattcgtacgaaccttacgttttttttttcaagcaacttcaataacccggtacaacctggcgaccacgacgccaccaccaacttcacgctgcaatcaaaactggttcagaattcattggcgacgactcttactattctatatccatttgctggtcgtttccgtaatgatgacacaattatttgtaaagatgatggtgcctttttcattgaagcaaaaactgacacgaagctgagtgattttcttgcccaaccggacctacctttagccatcatggataaattggtacctgtggcaactgacgctaaatacaatggctcccttttaattctaaaatttactttgtttggctgtggcggatccgctgtgacaataagtataacacacaaaataagcgatctagcaacgatactgactttattaaactgctggactgcattatctcgtggcggggatggaggtggcagttctccctttatacaaccagacttaaacttcatcgggaggccagtgccgtctacttctgaggttcccccaccgtcatcaggaaaaaattttatccctccgaacagcaagtatgtaactaaacgtttcatcttctccgccgcaaagatcaaagaattgaaagcgagggtaataaataaaatccgtaaagaagaagacaacgtgttcccttctagggtcgatgtggtgctagcgcttatatggaagtgtgccttggcaagtgtcaattcaggatcacgttcaggtaatgcccagaccttccgtccctccgtaatgatgcaggcggttaacctgaggaaccgtaccgaccctcccctgccggaatcaagcatagggaacctagctatcctattaccggtttgggtagagaaagaggaagacaccgagttacatgaacttgtcagcaggttattgacggtgaaggtaagggcgaatcgtcttaaaaaaaagtaccagggttatgaggatcccgagcaagtaattatttccatggagtctgattccgtcaaggagataatagaagtcaggaaaaaattaaaagatttttctacgtacgtcgcagcttccgtggtgaacgcccctctatacgatgttgattttggatgggggaagccagcttgggttacttccacgcccaatacagttatggctaactccatatacttgctggacaccaaagacgcgggcgggattgaagttttgatgaatatgttaaaggaggaagatatgatcgtgtttgaatcaaatcaggaattacttcagtccgcaatggttaatccgacaataatatga

SEQ ID NO: 196 - protein, 485 aa, Quillaja saponaria
MAEALKLKVVDEFQVSPPQDSVPTTTLPLSFFDIPFLFISQGKRIFFYEFPHPTHHFMEAVLPHLKQSLSLTLKHFFPVAANIVFPPQPNKPHVLFDSGSSSVSFTVAESTADFNYLITDSPRAVSSVDPFVPKIPPARVLDDGTRLFPLLAIQATVIPNSGFSICIRFHHGIGDGMAFQHFVRSWAFQCKKLISTKPEEEEEGGKHSSLPIFNNGSTIPDPNGVELCFLNFLWKWTPALLKMADGSGSEFNSTHNSVNVDATFKVTQTHIDKLKQLISLKFYNGNESESEPVEKAPLRLSTFVVSCALIWVCLAKSEENTYIDDEEPYNFTFVADCRGRFEFSSVPATYFGNCVAPVYATLKRKEIVGENGIVEAAKAIRNKIKELEKNGVLKVAERWMLDAIEKTTTKHNVMVAGSPKLGVYEMDFGWGSPKKSDVLGNPGSILISLSESKDKNLGGIEVGVGLEKTQMNNFIQTFDQTLKIL

SEQ ID NO: 197 - DNA, 1458 bp, Quillaja saponaria
  source 1..1458: mol_type = genomic DNA
atggctgaagctttgaaattgaaagtagttgatgaattccaagtttctccaccacaagattcagttcccacaaccactctccctctctctttttttgacattccatttctttttatctctcaagggaaacgtattttcttctatgaattccctcacccaactcatcatttcatggaagctgttcttcctcatctcaaacaatctctttccctcactcttaaacactttttcccagttgctgccaatattgtatttcctccacaacccaacaaaccccatgtcctctttgactccggttcctcctccgtctccttcaccgtcgctgaatccaccgccgatttcaactacctgattactgattccccacgcgccgttagttctgttgacccatttgtccccaaaatccctcccgctcgtgtcttggacgacggcacacggttgtttcctctcttggccattcaagccactgtcataccaaactctggattcagcatttgtatcagatttcaccatggaattggtgatggaatggcatttcagcattttgtaagatcctgggcatttcaatgtaagaaattaattagcacaaaaccagaagaggaagaagaaggaggaaaacattcttccctcccaattttcaataatgggtctaccattccagacccaaatggggtcgagctttgtttcttgaactttttatggaagtggacacctgcacttctaaaaatggcagatgggtccggatccgagttcaactcgacccataatagtgtcaatgttgatgccacattcaaggttacacaaacccatattgataagctaaagcaattgatctctctgaaattttacaatgggaatgagtcagagtcagagccagttgaaaaagcaccattacgtttatcaacctttgtagtatcatgcgccttaatttgggtttgtttagcaaaatcagaggagaacacttacattgatgatgaagaaccttacaattttacttttgtagctgattgtagaggtcgttttgagttttcttctgtacctgcaacatattttgggaattgtgtggctcctgtttatgcaacactaaagaggaaggagattgtgggagagaatgggattgttgaagcagcaaaagctataagaaataaaatcaaggaattggagaaaaatggagttttgaaagttgcagaaagatggatgttagatgctatagagaaaacaacaacaaagcataatgttatggttgctggttcaccaaaattgggtgtttatgaaatggattttggttggggaagtcctaagaagagtgatgtgcttggtaaccctggttctattttgatttctttgtccgaatcaaaggacaagaatctaggtgggattgaagtgggtgtgggacttgaaaagacccaaatgaataatttcattcagacatttgatcaaacattgaagatactctaa

SEQ ID NO: 198 - DNA, 1458 bp, synthetic construct
  source 1..1458: mol_type = other DNA
atggccgaggccttgaagctgaaggtggttgatgaattccaagtctccccaccccaggatagtgtccctacaacaactttacccttaagcttttttgacataccattccttttcataagccagggcaagcgtatctttttctatgagttccctcaccctacgcaccactttatggaagcggtcttgccacatcttaagcagtcattgtcccttactttgaagcattttttccctgtagcagcaaatatcgtgtttcccccccagcccaataaaccccacgtacttttcgatagtggtagctcatccgtttctttcacagtagccgagagtactgctgactttaactatttaataaccgattcaccgcgtgccgtctcttcagtagatcccttcgtaccaaagatcccgccagcgcgtgtgttggatgatggtactagattgttcccgctactagctattcaagccacagtaataccgaactctgggttcagcatttgtattagatttcaccatggaataggcgacggtatggccttccaacatttcgtgcgttcttgggccttccagtgtaaaaagctgattagtactaagcctgaggaggaagaggaaggtggtaaacactcctcactaccgattttcaacaacggatctacgatccccgacccaaatggggtagagctatgtttcctgaactttctttggaaatggacacccgcattgctgaaaatggcagacggtagtggaagtgaattcaactccacacacaacagcgtaaacgtagacgcaacatttaaggtcacacagacacacattgacaaactaaagcagctaatctctctaaagttttacaacgggaacgagtcagaatctgaacccgtggagaaggcccccttgaggttgagtacgttcgttgtctcatgtgcgctgatctgggtgtgtttggcgaaaagtgaggagaacacctacatagatgatgaagagccttacaactttacatttgttgccgactgtaggggtaggtttgagttctcaagtgtcccagcgacttatttcggcaactgcgtcgcgcctgtatacgcgaccttgaagcgtaaggaaatcgtcggagagaatggaatcgttgaagcggcaaaagccatcagaaacaaaatcaaagaacttgagaagaacggagtactaaaagtagcagagcgttggatgctagacgcaattgaaaagactacaacaaagcacaatgttatggtagcggggtctccgaaattaggtgtatacgaaatggactttggatggggtagtcccaagaaatcagacgttcttggaaatcctggttctattttgatctcccttagcgagagtaaggataaaaatttgggcgggatagaggtaggtgtcggactggagaaaacccaaatgaacaattttattcagacgttcgatcaaaccttaaagatactataa

SEQ ID NO: 199 - protein, 352 aa, Arabidopsis thaliana
MGSSVEQNILVTGGAGFIGTHTVVQLLKDGFKVSIIDNFDNSVIEAVDRVRELVGPDLSKKLDFNLGDLRNKGDIEKLFSKQRFDAVIHFAGLKAVGESVENPRRYFDNNLVGTINLYETMAKYNCKMMVFSSSATVYGQPEKIPCMEDFELKAMNPYGRTKLFLEEIARDIQKAEPEWRIILLRYFNPVGAHESGSIGEDPKGIPNNLMPYIQQVAVGRLPELNVYGHDYPTEDGSAVRDYIHVMDLADGHIAALRKLFADPKIGCTAYNLGTGQGTSVLEMVAAFEKASGKKIPIKLCPRRSGDATAVYASTEKAEKELGWKAKYGVDEMCRDQWKWANNNPWGYQNKLI

SEQ ID NO: 200 - DNA, 2028 bp, Arabidopsis thaliana
  source 1..2028: mol_type = genomic DNA
acatacgaaacatatattccttctcaaaacaaaatcctattagctaactctagtaccaaaacatttcaacatttacccaaattggatacctaaataaagttgtagatacacgtcatcaatttgatcataacgttatcggtaggtctctgctgtctctgtcacaatcttaggtatcgttaaaagataaatattaagtggacaactttaacgaaaaaacacaaaaaagttgattggacaacgttactatccagtttttagcgtgttatccaacccatagaaaatttatctctctgtattcccataatcctcgtagattttggtcatcactagtcatttatttacaaaaataggttaaaaatgtcattgcgtaaaaaaaaaagaagacttaatcttacattatttcccttttaattattaaaatccgccgagaaaagtttcaccatcaatgcgtaactgaaaatcgacgaatcaaagctataatacacttaaacggatcatcatcatcatcatcgttgtcatgatcatcactgccgtatttatagaaaccaacaaaaacatgaaagagcccactcatttttcctatttttcgctttgcgtgccttcttatcaacttgtaaacaaagctacaaatatctttaaataaggacccaactctttttcaattcctcccatcaatcttcttcatttgttcttctgttggtggtggtgatctagttttcaaagaatcgattttgccaagtgggttcttcttgataacctttcttcttctttgaaatgggttcttctgtggagcagaacattcttgttactggtggtgctggctttatcgggacgcatactgttgttcaacttctcaaagatggttttaaggtttcgatcatcgataattttgataactctgttatcgaagctgttgatagagttagggagcttgttggtcctgatctctccaagaagctcgacttcaatctgggtgatctaagaaacaaaggggacattgagaaactattctccaagcagagatttgatgctgtgattcattttgcgggtcttaaagctgtgggtgagagtgttgaaaaccctcgccgctactttgacaataacttggttggaacaatcaatctatatgagaccatggcaaagtacaactgcaaaatgatggtgttttcatcttctgccactgtttatggacaacctgaaaagattccatgcatggaagactttgaattaaaggctatgaatccttatggtcgtactaagctctttcttgaagaaatagctagagatattcaaaaggcagaaccggaatggagaattattctgctgaggtacttcaatcctgtaggagcacatgagagtggcagtattggtgaggatccaaaaggcatccccaataacctcatgccttacatccaacaagtggccgttggacgtttaccggaactcaatgtctatggacatgactatcccaccgaggatggtagtgcggtaagagactacatccatgtgatggatttagcagatggccatatcgctgcgctcaggaagctatttgctgatccaaagattggttgtactgcttacaatctagggactggtcaaggaacgtctgtgttagaaatggttgcagcttttgaaaaagcttccggcaagaaaatcccgattaagctctgtccgagaaggtcaggagatgcaacagcagtttatgcttcaacagagaaggctgagaaagaacttggctggaaggcaaaatatggagtggatgagatgtgcagagatcagtggaaatgggcaaacaataatccatggggttaccagaataagctttgaatttacttcttttgtttggagttaccatttctaattactcaaatctaaaagaaagaaatatacatacatatgatgatatagttgtgctttatattccacatgtatcgaactgatctcttacttcgatgaataaaatggaaagttgatttgattgaatttttagttttctttcactgaataaaaaggcttgttcaatgg

SEQ ID NO: 201 - DNA, 1059 bp, synthetic construct
  source 1..1059: mol_type = other DNA
atggggagtagtgtcgaacagaacattcttgtaacgggtggggcggggtttatcggcacacatacggtagtacagttattaaaagatggattcaaagtaagcataatcgacaatttcgacaattctgtaattgaagcagtggaccgtgtgagggagttggtcgggccagatttgagcaaaaaactagacttcaatttaggagatctaagaaataaaggagatattgagaaacttttttcaaaacagaggtttgacgctgttattcactttgccggcttgaaggccgtgggcgaaagcgtggaaaacccacgtaggtattttgataataatctggtagggaccatcaatctgtatgagacgatggcaaaatataattgcaaaatgatggtcttttctagttcagcaaccgtatacgggcaaccggaaaagatcccatgtatggaggactttgagttaaaagctatgaacccttacggacgtactaaactattcctggaggagatagctagagacattcaaaaagcggagcccgagtggagaattatattgctaaggtattttaatcccgtaggagcgcacgaatcaggcagtatcggggaagatcctaaaggcattccgaacaacttaatgccatacatccaacaggtagcagttggacgtctgcctgagcttaacgtgtacggacatgattatccgacggaggacgggtctgccgttcgtgattatattcacgtcatggatttagccgacggacatattgctgccctgaggaagctttttgcagatcccaaaataggatgcactgcttacaaccttgggaccggccagggaacatcagttctggaaatggtagctgcgttcgagaaagcttcaggaaagaagataccgatcaaattatgcccgcgtagaagtggtgacgccaccgccgtttacgcttcaacagaaaaagccgagaaagagcttggttggaaagcaaaatacggtgtcgacgagatgtgtagggatcaatggaaatgggcaaataataatccatggggttatcaaaacaaattgatctga

SEQ ID NO: 202 - protein, 417 aa, Arabidopsis thaliana
MLNLGRARTGRQNRSMSFEGLDFADPKKNNNYMGKIVLVMTLTAMCILLLNQSPTFNTPSVFSRSEPGVTHVLVTGGAGYIGSHAALRLLKDSYRVTIVDNLSRGNLGAVKILQQLFPEPGKLQFIYADLGDANAVNKIFSENAFDAVMHFAAVAYVGESTQFPLKYYHNITSNTLVVLETMAAHGVKTLIYSSTCATYGEPEKMPITEETPQVPINPYGKAKKMAEDIILDFSKNSIMAVMILRYFNVIGSDPEGRLGEAPRPELSEHGRISGACFDAARGIIPGLQIKGTDYKTVDGTCVRDYIDVTDLVDAHVKALEKAKPRKVGIFNVGTGKGSSVKEFVEACKKATGVDIKVDYLERRAGDYAEVYSDPRKIKEELNWTAKHTNLQESLKMAWRWQKLHRSGYGSSSLVSAY

SEQ ID NO: 203 - DNA, 1254 bp, Arabidopsis thaliana
  source 1..1254: mol_type = genomic DNA
atgctgaatttaggcagagctagaaccgggaggcaaaacagatctatgtcttttgaaggattggattttgcagacccaaagaagaacaacaattacatggggaagattgttttggtcatgacacttacagcaatgtgcattctcttgctcaaccaatccccaaccttcaataccccaagcgtgttttctcgaagtgaaccaggagttacacatgttctagtcactggtggtgcagggtatatcgggtcacatgcagctttacgccttctaaaagattcataccgtgtcaccattgtggacaatctatctcgagggaacctcggtgcagtcaagatcttgcaacaactgtttccggaacctggaaagcttcaattcatttatgctgatcttggagatgcaaatgctgttaacaaaatattttctgaaaacgcatttgacgctgtgatgcattttgctgctgttgcttatgttggagaaagcacacagtttcctctcaaatattatcacaatatcacatcgaacacattggtagttttagagacaatggctgctcatggtgtgaagactttgatatattcaagcacatgtgctacttatggagagcctgagaaaatgccaataacagaggaaactccacaggttccaatcaatccatatggaaaggctaaaaagatggcagaagatatcatcttggatttctccaagaattctattatggcagttatgatcctaagatatttcaatgtgattgggtctgatcctgagggtagactaggtgaagctccaagacctgaactcagtgagcatggacggatctctggtgcttgctttgatgctgcacgaggcatcattcccgggctgcagattaaaggaacagactacaagactgttgatgggacttgtgtacgtgattacattgatgtcaccgacttggttgatgctcatgttaaagctcttgaaaaggcgaaaccccgcaaagttggtatcttcaatgttggtaccgggaaaggtagctcggtgaaggagtttgtggaagcgtgcaaaaaggcgaccggcgttgatataaaagtcgattacttggaaagacgggcaggagattacgcagaggtttacagcgatccaagaaaaatcaaagaagagctgaattggacagctaagcacaccaatcttcaagaaagtttaaagatggcatggagatggcaaaagcttcatcgcagtggctatggatcttcttccttggtttctgcatactga

SEQ ID NO: 204 - DNA, 1255 bp, synthetic construct
  source 1..1255: mol_type = other DNA
atgttaaatctgggtagagccaggaccgggagacaaaacaggagcatgtccttcgaggggttagatttcgcggacccaaagaagaacaacaattacatggggaaaattgtactagtaatgacccttacggcgatgtgtattctgttacttaaccaatcccctacatttaatacaccaagcgtattctcccgttccgaaccgggcgtcacgcacgttttggttacaggcggtgctggctacatcggcagtcatgccgctctacgtctattgaaggattcttacagagtgactatcgtcgataatctatccagagggaacttgggagctgtgaagatacttcagcaactgttcccagaacctgggaagttacaatttatatacgccgaccttggagatgccaacgcagtcaataaaatattctctgaaaacgcctttgatgccgtcatgcatttcgctgcggtggcttatgtcggtgaatcaacgcagtttcctctaaagtactatcataacatcaccagtaatacacttgttgtattagaaacgatggcggcgcacggagtcaagacattaatttactcatctacttgtgccacctatggagagcccgaaaagatgcctattacagaagaaactccccaagtgcccataaacccctacggtaaagccaagaaaatggctgaagacattatcttggacttcagcaagaattcaattatggcagttatgatactgagatatttcaatgtaatagggagcgacccggaggggcgtttgggggaagctcccagacctgaactatctgaacatggaagaataagcggtgcttgttttgacgcggcaagggggatcatcccaggtcttcagattaagggaacagattacaaaacagtggatggcacctgtgtacgtgattacatagatgtaaccgatcttgtcgatgcccacgttaaggcattagaaaaggcaaagccgagaaaggttggaatcttcaacgtgggtactggtaaggggtcctctgtaaaagagtttgtagaggcttgcaagaaagctaccggagtggatatcaaggtggattatcttgaacgtcgtgcgggcgactatgctgaagtttattctgatcccagaaagataaaagaggagcttaattggaccgctaaacacacgaatttacaggagtcactaaaaatggcgtggcgttggcagaagttacatcgttccggttatggtagctcctccttagtcagcgcttactaaa

SEQ ID NO: 205 - protein, 351 aa, Arabidopsis thaliana
MGSSVEQNILVTGGAGFIGTHTVVQLLNQGFKVTIIDNLDNSVVEAVHRVRELVGPDLSTKLEFNLGDLRNKGDIEKLFSNQRFDAVIHFAGLKAVGESVGNPRRYFDNNLVGTINLYETMAKYNCKMMVFSSSATVYGQPEIVPCVEDFELQAMNPYGRTKLFLEEIARDIHAAEPEWKIILLRYFNPVGAHESGRIGEDPKGIPNNLMPYIQQVAVGRLPELNVFGHDYPTMDGSAVRDYIHVMDLADGHVAALNKLFSDSKIGCTAYNLGTGQGTSVLEMVSSFEKASGKKIPIKLCPRRAGDATAVYASTQKAEKELGWKAKYGVDEMCRDQWNWANKNPWGFQKKP

SEQ ID NO: 206 - DNA, 1056 bp, Arabidopsis thaliana
  source 1..1056: mol_type = genomic DNA
atgggttcttctgtggaacagaacattttggtgactggtggtgctggattcattggaacacatactgttgttcagcttttgaatcagggttttaaggttacgatcattgataatcttgataactctgtcgttgaagctgttcatagggttagggaacttgttggtcctgatctctctaccaagcttgaattcaatctgggtgatctaagaaacaaaggagatattgagaaactcttttccaatcagagatttgatgctgtgattcactttgctggtcttaaagctgtgggagaaagtgttggaaaccctcgtcgttactttgataataacttagttggaactatcaatctatatgagaccatggcaaaatacaactgcaaaatgatggtgttttcgtcgtctgcaacagtttatggtcaacctgaaatagtcccatgtgtggaagactttgagttacaggctatgaatccttatggtcgtactaagctgtttcttgaagagatagctagagacattcacgctgcggaaccagaatggaagataattcttctgaggtacttcaatccggttggagctcacgagagtggaagaattggagaagatccaaagggcataccgaataatctcatgccttatatccaacaagtggcggttggaagattacctgaactcaatgtatttggacatgattatcctaccatggatggtagcgcggttagagactacatccatgtaatggatttagcagatggccatgtggctgcgttaaacaaattgttttcagactcaaagattggctgtactgcttataatcttggcactggtcaaggaacttctgtcttagaaatggtttcttcttttgaaaaagcttctggcaagaaaatacctatcaagctatgtccaagaagagctggagatgcaacagctgtttatgcttcaactcagaaagctgagaaggaacttggctggaaggcaaaatatggagttgatgagatgtgcagagatcaatggaactgggcaaataagaatccatggggtttccagaagaagccttga

SEQ ID NO: 207 - DNA, 1056 bp, synthetic construct
  source 1..1056: mol_type = other DNA
atggggtcctccgtagagcaaaacatacttgtgactggcggtgccggctttatagggacacataccgtggtgcagcttctaaatcagggcttcaaggtcacaattatagacaacctagataattctgtcgtagaagccgttcacagggtaagagaattggtggggccagacctttccacgaaactggaattcaacttgggggatttgagaaacaagggggacatcgaaaaattattctcaaaccagcgttttgatgccgtgatacacttcgccggtctaaaggcggtaggggaaagtgtcgggaaccccaggcgttactttgataacaacctggtaggtactataaacctgtatgaaacaatggccaagtataattgcaaaatgatggtctttagcagttctgccaccgtgtacggtcagccagaaatagtaccgtgcgtggaagattttgagctgcaagctatgaatccatatgggagaaccaaattattccttgaagaaatcgccagggacatacacgccgctgagccagaatggaagataatcctgctgaggtatttcaatccagtcggcgcccacgaatctggtcgtatcggtgaggacccaaagggcattcctaacaatttgatgccatacatccaacaggtggcagtagggaggttacccgagttaaatgttttcgggcatgattacccaactatggacggctctgctgtaagagattatatacacgtcatggacttagccgatggccatgttgccgcattaaataaactttttagtgacagcaagattgggtgcacggcctacaacttggggaccgggcagggtacgtctgtcttagaaatggtctcctcctttgaaaaggcatcaggcaaaaaaatacccataaagctatgtccgagacgtgccggagacgcaacagcggtctatgcttcaacacagaaagccgagaaagagctaggatggaaagccaaatacggagtggatgaaatgtgtagggatcaatggaactgggcgaacaagaacccatggggttttcaaaagaaaccatag

SEQ ID NO: 208 - protein, 357 aa, Arabidopsis thaliana
MVEPANTVGIPVNHIPLLKDELDIVIPTIRNLDFLEMWRPFLQPYHLIIVQDGDPSKTIAVPEGFDYELYNRNDINRILGPKASCISFKDSACRCFGYMVSKKKYIFTIDDDCFVAKDPSGKAVNALEQHIKNLLCPSTPFFFNTLYDPYREGADFVRGYPFSLREGVSTAVSHGLWLNIPDYDAPTQLVKPKERNTRYVDAVMTIPKGTLFPMCGMNLAFDRELIGPAMYFGLMGDGQPIGRYDDMWAGWCIKVICDHLGLGVKTGLPYIYHSKASNPFVNLKKEYKGIFWQEDIIPFFQSAKLTKEAVTVQQCYMELSKLVKEKLSPIDPYFDKLADAMVTWIEAWDELNPPTKA

SEQ ID NO: 209 - DNA, 1567 bp, Arabidopsis thaliana
  source 1..1567: mol_type = genomic DNA
tctcccatcacctccgatctcaaatctcacaatcttttctctccaaattctcttctctctgaatcaatccaatcatggttgagccggcgaacaccgttggaattccggtgaaccacatcccattgttgaaggatgagctcgatatcgtgatccccacgatccgtaacctcgatttcctggagatgtggaggccttttcttcagccttaccatctgattatcgtccaagatggagatccatcgaagaccattgctgtccctgaagggttcgattacgaactctacaacaggaacgacatcaaccgtatccttggtcctaaagcttcctgcatttccttcaaggactctgcttgtcgttgcttcggctacatggtctccaagaagaagtacatcttcactattgatgacgattgcttcgttgctaaggatccatctggaaaagctgtgaacgctcttgagcaacacatcaagaaccttctctgcccatcaactccatttttcttcaacaccttgtacgacccataccgtgaaggtgctgacttcgtccgtggataccctttcagtctccgtgagggtgtttccaccgctgtttcccacggtctgtggctcaacatccctgattacgatgcccctacccaacttgtgaagcctaaggaaaggaacacaaggtatgtggatgctgtcatgaccatcccaaagggaactcttttccctatgtgtggtatgaacttggcctttgaccgtgagctcattggtccggctatgtactttggtctcatgggtgatggtcagcctattggtcgctacgacgatatgtgggctggatggtgtatcaaggtgatctgtgaccatttgggattgggagtgaagacaggtttgccctacatttaccacagcaaagccagcaacccgtttgtgaacttgaagaaggagtacaagggaatcttctggcaggaggatatcattcctttcttccagagcgcaaagctcacgaaagaagctgtgacagttcaacaatgctacatggagctgtccaagttggtgaaggagaagctaagccccattgatccttactttgacaagcttgcagatgctatggtcacttggattgaagcttgggatgagcttaacccacccactaaagcttgagcagcaaaaaaaccaccaccgcagttttggttattagctcaacatatcatctatcttctccgttttgtttttgttttgtcttttctcaaattttccggcgattcttcagttcgtttttaagttgccggagtttatttaaatagtgaatggtgtagttctatttatgaccgagacaatctctcttttgaggtttttcgtttttgtttcaataataaaaaatcatatcccttagtcgttggtgtctttgcttgctggtttgttgactgttattttggatcaaattagttgaaagacaggaacagattccaatcaaatcaagccttaaacatatttgatttagtagaacccagttggtgtttttggctgtatccgacatgttatgtaaaaatggccaagagactccaacatcattattaaaa

SEQ ID NO: 210 - DNA, 1074 bp, synthetic construct
  source 1..1074: mol_type = other DNA
atggtcgaaccagccaacaccgtgggcatacctgttaatcacataccgttattgaaggacgaactggatattgtgatccctacgatccgtaatctagatttcctggagatgtggcgtcctttccttcagccttatcatctaataatagtgcaggatggcgatcctagcaaaacaatagccgtaccggaaggctttgactacgagctgtacaataggaatgatatcaacagaatactgggccccaaggcttcttgtatttcttttaaagactcagcttgtagatgcttcggctatatggtatccaagaagaagtacattttcactattgacgacgactgtttcgttgccaaggacccttctggcaaagcagtgaacgctctggaacagcatatcaagaatttactgtgcccatccaccccgtttttctttaatactctatacgacccgtatagagagggcgctgactttgtacgtgggtatcctttcagtttgcgtgagggagttagcaccgccgtttcacacggtctttggctgaacattcctgattacgatgcgccaacacaactagtgaaaccaaaggaaaggaatacacgttacgtcgatgcggtaatgaccatccctaaaggtactctgttcccaatgtgtgggatgaatttggcgttcgatagggaactaataggcccagcgatgtattttggtttaatgggagacggacagccaatcggaagatatgatgatatgtgggctggctggtgcatcaaggtgatctgtgaccacttaggattgggtgtcaagacaggactgccctacatttaccactctaaagctagcaatccatttgtcaacttgaaaaaagaatacaaaggaattttttggcaggaagacataattccgtttttccaatctgcaaaactaacgaaagaggcggtaacggtgcagcagtgctacatggagttatcaaagttggtcaaagaaaaattgtcccctatagatccatactttgacaaattggcggacgcaatggttacctggattgaagcatgggatgaactaaatcccccaacgaaggcctaa

SEQ ID NO: 211 - protein, 371 aa, Hordeum vulgare
MAGTVTVPGSSVPSTPLLKDELDIVIPTIRNLDFLEMWRPFFQPYHLIIVQDGDPSKAIKVPEGFDYDLYNRNDINRILGPKASCISFKDSACRCFGYMVSKKKYVFTIDDDCFVAKDPSGKDINALEQHIKNLLSPSTPFFFNTLYDPYREGADFVRGYPFSLREGAPTAVSHGLWLNIPDYDAPTQMVKPRERNSRYVDAVLTIPKGTLFPMCGMNLAFDRQLIGPAMYFGLMGDGQPIGRYDDMWAGWCVKVICDHLSLGVKTGLPYLWHSKASNPFVNLKKEYKGIFWQEDIIPFFQNATLSKECDTVQKCYISLSEQVREKLGKIDPYFLKLADAMVTWIEAWDELNPSATPDAVAAAGNGKAAAK

SEQ ID NO: 212 - DNA, 1499 bp, Hordeum vulgare
  source 1..1499: mol_type = genomic DNA
atagaaaggcaggcatcggtggacaagaaagtcttcttcatcgtggagcgctatataaccccctaattttccaagcccaaacccacacctctctctactcccccacccctatcaccgcctgctggaacagctagatccagggctcgagatcgagatccccggccaggaagcaatggcagggacggtgactgtgcccggatcgtcggtgccatcgacgccgctgctcaaggacgagctggacatcgtgatcccgacgatccgcaacctcgacttcctcgagatgtggcgccccttcttccagccgtaccacctcatcatcgtccaggacggcgaccccagtaaggccatcaaggttcccgagggcttcgactacgacctttacaaccgcaacgacatcaaccgcattctcggcccaaaggcctcatgcatctccttcaaggactccgcgtgccgctgcttcggctacatggtctccaaaaagaagtacgtcttcaccatcgacgacgactgcttcgtggccaaggacccatctggcaaagacatcaatgctcttgagcagcacatcaagaacctcctcagcccgtcaaccccgtttttcttcaataccttgtatgacccctaccgtgaaggcgcggactttgttcgtggataccccttcagccttagggagggtgcacccactgctgtctctcatggcctttggctgaacatccctgactatgatgcacccacacagatggtcaaacctcgtgagaggaacagcaggtatgttgatgccgtcctgactattcccaagggaacattgttccccatgtgtggcatgaaccttgcttttgaccgtcagctcatcggccctgcaatgtactttggcctcatgggtgatggccagcctattggtcgctacgatgacatgtgggctggatggtgcgtcaaggtcatctgcgaccacttgagccttggagtgaagactggcctgccatacctgtggcacagcaaggctagcaacccatttgtcaacttgaagaaggagtacaagggcatcttctggcaggaggacatcattccctttttccagaacgccaccctttccaaggagtgtgacacagtccagaagtgctacatctcactctcggagcaggtaagggagaagcttggcaagattgacccttacttcctgaagcttgctgatgccatggtcacctggatcgaggcctgggacgagctgaacccatctgctactcctgatgccgttgctgctgctgggaacggcaaggcagcagccaagtagctagcttgggaagtcaagagtgtatggttacggcctggatatagctcttttcgaggaagagattactgaagcagcttttataattctttcatgttatgctggtggttcttttgtagctggataaggatttgttatcatttacataattcaagacaataatatttatcatgtaattttgagaaatcaagcaccgtttgttgttacagtgaaa

SEQ ID NO: 213 - DNA, 1116 bp, synthetic construct
  source 1..1116: mol_type = other DNA
atggcggggaccgttacggtgccggggtcttctgttccttctaccccgctgcttaaggacgagctggacattgtaatccccactataagaaacctggatttcctggagatgtggaggcctttttttcaaccttatcacctaataatcgttcaggatggcgaccctagtaaggccatcaaagtccccgaagggttcgattacgacttgtacaataggaatgatataaacaggatactggggcctaaggcatcctgtatttcatttaaagatagcgcgtgcagatgcttcgggtacatggtttctaagaaaaaatacgtttttacaattgacgatgactgcttcgtggcaaaagacccttcaggtaaagacatcaacgccctggaacagcacatcaagaatcttctgtccccctcaaccccttttttctttaatactctatatgacccatatagggagggagccgatttcgttaggggatatcctttctccctacgtgaaggtgctccgaccgcggtgtctcatggtttgtggttaaacattcccgattatgatgcaccgacccagatggttaaaccgcgtgagcgtaattcacgttatgtggatgcggtgctaacaatccccaaaggcactctattcccaatgtgtggaatgaatttggctttcgataggcagttgatcgggcctgctatgtactttgggctgatgggcgatggacagcccattggaagatacgacgacatgtgggcaggctggtgtgtaaaagttatctgcgatcatctaagcttaggtgtgaaaactgggttgccttacttgtggcattccaaggcatcaaacccattcgttaatttgaaaaaggagtacaagggcatcttttggcaggaagacatcattcctttttttcagaacgcaaccctaagcaaggaatgtgacaccgtgcaaaagtgttacattagtttaagtgagcaagtcagggagaaactagggaagatcgatccgtacttccttaaactggcagacgccatggtaacttggattgaagcctgggatgaactgaacccatccgcgacccctgacgctgttgcagcggcaggcaacgggaaagccgctgctaagtag

SEQ ID NO: 214 - protein, 1048 aa, Arabidopsis thaliana
MDYKDDDDKMSERAKLQKKKKVTEDLLDSLLLLPPWRNFAISQSHQSIIESSSQRLPEKMRIDENEGVSASSKHLVFAYYVTGHGFGHATRVVEVVRHLIAAGHDVHVVTGAPDFVFTSEIQSPRLKIRKVLLDCGAVQADALTVDRLASLEKYVETAVVPRAEILETEVEWLHSIKADFVVSDVVPVACRAAADAGIRSVCVTNFSWDFIYAEYVMAAGYHHRSIVWQIAEDYSHCEFLIRLPGYCPMPAFRDVIDVPLVVRRLHKSRKEVRKELGIAEDVNVVILNFGGQPSGWNLKETSLPTGWLCLVCGASETLELPPNFIKLAKDAYTPDIIAASDCMLGKIGYGTVSEALSYKVPFVFVRRDYFNEEPFLRNMLEFYQCGVEMIRRDLLMGQWTPYLERAVSLKPCYEGGINGGEIAAHILQETAIGRHCASDKLSGARRLRDAIILGYQLQRVPGRDIAIPEWYSRAENELGQSAGSSPTVQANENNSLVESCIDDFDILQGDVQGLSDTCTFLKSLAMLDAIHDSEKSTEKKTVRERKAAGGLFNWEEEIFVARAPGRLDVMGGIADYSGSLVLQMPIREACHVAVQRNLPGKHRLWKHAQARQQAKGQVPTPVLQIVSYGSEISNRAPTFDMDLSDFMDGDEPISYEKARKFFAQDPAQKWAAYVAGTILVLMIELGVRFEDSISLLVSSAVPEGKGVSSSAAVEVASMSAIAAAHGLSIDPRDLAILCQKVENHIVGAPCGVMDQMTSSCGEANKLLAMICQPAEVVGLVEIPNHVRFWGIDSGIRHSVGGADYRSVRVGAYMGRKMIKSMASSILSPSASSANGGNPEELEDEGIDLLEAEASLDYLCNLSPHRYEARYADKLPDIMLGQTFIEEYADHDDPVTVIDQKRSYSVKAPARHPIYENFRVKTFKALLTSATSDEQLTALGGLLYQCHYSYSACGLGSDGTNRLVQLVQGMQHNKSNSEDGTLYGAKITGGGSGGTVCVVGRNSLRSSQQILEIQQRYKAATGYLPLIFEGSSPGAGKFGYLRIRRRISL

SEQ ID NO: 215 - DNA, 3500 bp, Arabidopsis thaliana
  source 1..3500: mol_type = genomic DNA
ccttcgcatcataataaattattaataatttataacgattttaaggaagaaaaaaaagcttttaaaatcggagaattaatttcggttagttcagatttatgagtgaaagagcaaaattgcaaaaaaaaaaaaaagtcactgaagatttattagactctcttcttcttcttcctccttggcgcaatttcgcaatttcgcaatcgcatcagtcaattatcgaatcgtcctcccagagattaccggagaaaatgaggattgatgagaacgaaggtgtctctgcttcaagcaagcacctagtcttcgcttactacgtcactggacatggtttcggtcacgccactcgagtcgtcgaggttgtccgtcacctgatcgcggcggggcatgatgttcatgttgtcaccggcgcgcctgattttgtcttcacatctgaaattcagtctccgaggctcaaaattcgaaaggttcttttggattgtggggctgtacaagctgatgcattgaccgttgatcgtcttgcctcgttagaaaagtatgtggaaacagctgtggttcctcgagctgaaatattggaaactgaagtggagtggcttcattctatcaaagctgattttgtggtgtctgatgttgtccctgtcgcatgccgtgctgcggctgatgctgggatacgttctgtttgtgtcaccaatttcagctgggattttatctatgctgagtacgtgatggctgctggatatcaccatcgctccatcgtttggcagatagctgaagattattctcattgcgagttcctaattcgcctcccaggatattgtccaatgcctgcttttcgtgatgtcatcgatgtaccgttagttgtgaggagacttcacaaatctcgcaaggaggtgaggaaagaactcggcattgctgaagatgtgaatgttgttatactcaattttggtggacagccctcagggtggaacttgaaggaaacatcactaccgactgggtggctgtgtctggtttgtggcgcatctgagaccctggagcttccaccaaattttataaagcttgctaaggatgcttatacgcctgatattatagcggcatctgactgtatgcttgggaaaattggttatggcactgtcagtgaagccctatcctacaaggtgccgtttgtctttgtacgcagagattatttcaatgaggaaccttttctgagaaatatgcttgagttttatcaatgtggtgttgagatgatcaggagagacctattaatgggccaatggacgccatatcttgaacgtgcagttagcttgaaaccttgctatgagggtggcatcaatggtggtgagattgcagctcacatcttacaggagacagccattggaagacactgcgcatctgataagttaagtggtgcaagaagattacgtgatgcaattatcctggggtatcagctacagagagtccctggaagagacattgctattccggagtggtattcaagggctgaaaatgaactaggtcaatctgctggatcatcacctactgttcaagcgaatgagaacaactcattggtggagtcgtgcatcgatgattttgatatccttcaaggtgatgttcaaggtctttcagatacgtgtacatttcttaagagtttagctatgctagatgctattcatgattctgaaaagagtacggagaagaagaccgtgcgtgaaagaaaagctgctggtggtctcttcaattgggaggaagaaatctttgttgccagagcacctggaaggttagatgtgatgggtggaattgctgactattcaggaagccttgtcttgcagatgccaataagggaagcttgccatgtagctgttcaaagaaaccttcctggaaaacatagactgtggaaacatgcccaagcccgacagcaagctaagggacaagtaccaacaccagttctgcaaattgtctcatatggatcagagatcagcaatcgtgcccccacttttgacatggatttgtctgattttatggatggagatgaaccaatttcttatgaaaaagcaagaaagttctttgctcaggatccagcacaaaagtgggcagcatatgttgctgggacaatcttggtattgatgatagaacttggtgtgcgttttgaggatagcatcagcttgctggtttcttctgctgtacctgaaggtaaaggtgtatcttcatctgctgctgtggaggtggcgagcatgtccgcaatagctgctgcacatggactgagtatcgaccccagagatcttgctatactctgccaaaaggtggagaatcacattgttggagctccatgtggtgttatggatcagatgacttcttcctgtggggaagccaataaactgttggccatgatctgccaacctgcagaagtagttgggcttgttgagattcccaaccatgtcagattttggggaattgattccggaattcgacacagcgttggaggtgcagactataggtcggtcagagttggcgcttacatggggagaaagatgatcaagtcaatggcgtcttctattctgtcaccatcagcgtcaagtgctaatggaggcaacccggaagaactagaggatgaagggattgacctacttgaggccgaagcttcattagattacttgtgcaatttgtcgcctcacagatatgaagcccgttatgcagataagcttccagatatcatgttgggtcagacattcattgaggaatatgcggatcatgacgatccggtcacagtgattgatcaaaagcgttcttacagtgtaaaagctcctgccagacaccctatttacgaaaacttccgggtcaagacatttaaagctctattgacttctgcaacatcagatgagcaacttactgctcttggaggacttctttatcagtgtcactatagttacagcgcatgcggactaggatcggatggaaccaaccggctggtgcagttagtacaagggatgcagcataacaagtctaattctgaggatggaacattgtatggagccaaaatcacaggcggtggatcaggtggaacagtctgcgtcgtaggccgtaactcattgcgcagcagccaacaaattctcgagattcagcagagatacaaagctgcgacagggtatttaccgctgatattcgaaggttcgtcacctggagctggcaagtttggttacctccggatccgacgtcgtatctctctctgattcccttcccattcaacttctccctaatttggtaaacttttatttatttttatgccacgtttcttaaatcatattcatataggagtatcatctcacaaaataagtaggtccgttgttctccactttcaaaataataatgatgataataatcatcttttatttagataaaaatgatatgtatatactgcaatgttatgttaaacatcttatgcaaaagaaaacaactactcttcttttattattgttttatgttaatctcgaatgtgttcaatgtgtttgcat

SEQ ID NO: 216 - DNA, 3120 bp, synthetic construct
  source 1..3120: mol_type = other DNA
atgtccgagagagcgaagttgcagaaaaagaaaaaagtcacagaggacctactggatagtttactgttgttgccgccgtggagaaactttgccatctcacagtcacatcaaagtatcatagaaagctcctctcaacgtttgcccgagaaaatgaggattgacgagaacgaaggcgtctcagcaagctcaaaacacctagtgtttgcttactacgtgactggccacggatttgggcacgctacccgtgtagtagaggttgtaaggcatctaatagccgctggccatgatgtacatgtcgtgacgggcgcaccagatttcgtattcacttccgaaatacagtcacccaggttgaagatacgtaaggtcttactagactgcggcgcagtccaagccgatgccctgacagtggacaggttggcatccctggaaaagtatgtggagactgcggtagttccgcgtgcagaaatattagagactgaggttgaatggcttcactctattaaggcggacttcgtagtatcagatgtggtgcccgtcgcttgcagggcagccgcagacgccggtatccgttccgtatgtgtaactaatttcagttgggatttcatttatgcagagtatgtcatggctgctggctaccatcataggtccatcgtatggcagatagcggaagactattcacattgcgaatttttaattcgtttgccagggtattgtccaatgcctgcttttagggatgtgattgatgttccccttgtggtcagacgtctgcataaatctaggaaggaagtaagaaaggagttgggaattgccgaagatgtaaatgtcgtcatcctaaactttggtggtcagccttcaggatggaacttaaaagagacttctctaccaacgggctggctttgcctggtgtgtggagcatctgaaactctagaactgccccccaactttatcaaattggccaaggacgcatacacgcccgatataattgctgccagcgattgcatgttaggtaaaatagggtatgggacagtttctgaagcactatcttataaagtcccttttgtgttcgtccgtagggactatttcaatgaagaacctttccttcgtaacatgctagagttttatcagtgcggagtggaaatgataagacgtgacttgctaatggggcaatggactccgtacctagaacgtgcagtgagcctaaagccgtgttacgagggcggtatcaatggtggtgaaatcgcagcccacatattacaagagactgcgatagggagacactgtgcctctgataaactatctggggcccgtcgtttgcgtgacgccattatcctggggtatcagctacagcgtgtcccggggagagatatcgctatcccggaatggtatagtcgtgcggagaatgaacttgggcagtcagcaggatcatctccgaccgtgcaggcgaacgagaataactcactagtggaaagctgcattgatgactttgatatactgcaaggagatgtgcagggtttaagcgacacatgcacattccttaagagcttagcgatgctagatgccattcatgacagcgaaaagtctaccgaaaaaaagactgtaagggagagaaaggccgcggggggcttgtttaactgggaggaagaaatatttgtcgcgcgtgcaccagggagattggacgtaatggggggtatcgcggactattctgggtcattagtgctacaaatgccgattcgtgaggcatgtcacgtcgcagtgcagaggaatctaccaggtaagcacagactgtggaaacatgctcaggcgagacaacaagcgaaaggacaagtccctacgcctgttctacagattgtaagttacggttccgagatcagcaatcgtgctcctacatttgacatggacttgtccgactttatggatggagatgaaccgatttcatacgaaaaagcccgtaaattttttgcgcaagatcccgcacagaaatgggctgcgtatgtcgcgggcactattcttgtgctaatgatagaactaggggtccgtttcgaagacagtatcagtctactagtgtcctcagcagtgccagaaggtaagggtgtaagctcatctgcagcagtggaggtggcgagtatgagcgctattgcggcagcgcacggcttgtccatcgatccgagggacctagctatattgtgccaaaaagtcgagaatcacatcgttggcgcgccgtgcggggtgatggatcaaatgacctcctcatgtggggaagctaacaaacttctggccatgatatgccaaccggccgaggtcgtcgggctggtagagatacccaatcacgttcgtttttgggggatcgactcaggcataagacactccgtcggtggagctgattatcgtagtgttagagtcggcgcctacatggggaggaagatgatcaaatctatggcatcaagcatcttgagcccatctgccagcagcgccaacggtgggaatccagaagagctggaggacgaaggtatcgatctattagaggccgaggcttcattagactacttgtgcaacctttctccgcaccgttacgaagcgaggtacgcagataagttaccagacataatgctggggcagacattcattgaagaatacgctgatcatgacgatcccgtaactgtgattgatcagaaacgttcctactccgtaaaagctccggctaggcacccaatttacgagaattttcgtgtaaagacgtttaaagcacttctgacgtctgctacaagcgatgaacaattgacagccttaggcggtttgttgtatcagtgccactatagctacagtgcatgtggcctgggaagtgatggaacgaacagactggtacagctagtccaggggatgcagcacaataaaagtaacagcgaggatgggacgctgtatggcgcaaaaataaccggagggggatcaggtggaacggtgtgtgtcgtcggaaggaattctttaagaagctcccaacagattctagagatccagcaacgttataaagcagccacgggctatctgccattgatattcgaaggctcctcccctggtgcaggcaagttcggatatcttaggatcagaaggaggataagtttataa

SEQ ID NO: 217 - protein, 374 aa, Leptospira interrogans
MKITYYVSGHGFGHISRSMEIILYLLRSFPDLTIDLVTVREKFLDTIFLSEEDTKNLRRLQIRKRSLDVGMIQKDSLSIDTVATEAAIEEFNLQKSYIQISEIESCLDFGTELIISDSASLPFKIADKLKIPSLFIGNFTWDFIYSGYAKESSIFEKATRSIYEEYYHATFGLLLPFNCPANSLAEQKQIGLVGRKPFLNKNEAKEFFKLPKDKIHLLFSFGAYGVETSRFDWEKFDPEKYTIVLSGTDFDLLKIPNKQKNGIIQLSNLHYPDLLTACDFVITKPGYGILSEAVYAKTPVLYTDRGNFPEVPYLHKSLTEEIPSSYISNEDLFLFRLDKPLQKANVWKGKSSTLFERDGREDVKHAVAVFLKLI

SEQ ID NO: 218 - DNA, 1125 bp, Leptospira interrogans
  source 1..1125: mol_type = genomic DNA
atgaaaatcacttattacgttagcggtcatggatttggtcatatcagccggtccatggaaatcattctttatttacttagaagttttccagatttaacaatcgatctcgtaaccgttcgagaaaaatttctagatactattttcctttcggaagaagataccaaaaatctaaggcgtctgcaaattcgtaaaaggtccttggacgtgggtatgatccaaaaagattctctttccatagatactgtcgccacagaagcagctatcgaagaattcaatttacaaaaatcatatattcaaatttctgaaattgagtcttgtctggactttggaacggaattaatcatttccgattcggcatctcttccgtttaaaatcgcggacaaactcaaaatcccgtccctatttataggaaactttacctgggattttatctactctggttatgcaaaagaatcttcaatttttgaaaaagcgactcggtcaatttacgaggagtactatcacgctacattcggcctacttcttcctttcaattgtcctgcgaattccctagcggaacaaaaacaaattggtcttgtaggaagaaaaccgtttttaaacaaaaacgaggctaaagaattttttaaacttccaaaagataaaattcatcttttattctcgtttggagcttatggagtagaaacgtctcggtttgactgggaaaaatttgatccggaaaaatacacgatcgtactttcaggaacagatttcgatctattaaaaattcctaataaacaaaaaaatggaattatacaactttccaatctccattacccggatcttttgaccgcctgcgattttgtaattacaaaacctggttatggaattttaagcgaagccgtttacgcaaaaactcccgttctttataccgatcggggaaactttccggaagttccctatcttcacaaaagtttaacagaagaaattccatcctcgtatatctctaatgaagatttatttttatttcgattggataaaccattacaaaaagcgaatgtatggaaaggaaaatcttctactctttttgaaagagatgggagagaagacgtaaaacacgcagttgcagtttttctaaaattgatttag

SEQ ID NO: 219 - DNA, 1125 bp, synthetic construct
  source 1..1125: mol_type = other DNA
atgaagataacatattatgtgtccgggcacggctttgggcatatctcacgttccatggaaatcatattatacttacttaggtcattccctgatttgaccatcgatcttgtaactgtcagagagaaatttcttgatacgattttcctatctgaagaagacacaaagaacctacgtcgtttacagatccgtaaaaggtccctagatgttggtatgatccaaaaggacagtttgtccatagatacagtggccaccgaagcggctattgaggaatttaacttgcaaaaaagctacatccaaatctcagaaatagagtcctgtctagacttcggaacggagttaataatctctgatagcgcttccttaccttttaagatcgcggacaagcttaagattccttcactgtttataggaaattttacgtgggattttatatattcaggctacgcaaaagagtcctccatatttgagaaggccaccaggagtatatatgaggaatattaccatgcaacattcggtcttttgttgccgtttaactgccccgctaatagtctagctgaacagaagcagattggccttgtcggcagaaaaccattcttaaataagaatgaagcgaaggagttctttaagttaccgaaggataaaatacacctgctgtttagttttggggcgtatggggtggagactagccgtttcgattgggagaagttcgacccggagaagtacacaattgttttaagtggcactgatttcgatttattaaaaataccgaataagcaaaagaatgggataatccaactatccaacttacattacccggacctgttaactgcgtgcgactttgtcataacgaaaccgggctatggtatcctgagtgaagcggtctacgccaaaacacccgtgttatatacggaccgtggaaattttccagaggtgccctatctgcataagtctttgacggaggaaataccctcaagttacattagtaatgaggacctatttctgttcagacttgacaaacctttacagaaggcaaatgtgtggaaggggaagtccagcacactgtttgagagggatggtagggaagacgttaagcacgcagtagctgtttttctgaaacttatatga

SEQ ID NO: 220 - protein, 630 aa, Penicillium rubens Wisconsin
MPNPTGNDAVGGMTKNVTGEIKNPLAGIPHDQLMANVTAYATEYDLESILPVLKKGALVAQSPAGIEDLSELNDDDRRILYAEHTRRWKHPFALYYTIVLNSISAAIQGWDQTGSNGANLTFDVQFGIPNNSPQCPDPETCKRNQWIVGFINASPYITICLFVAWLSDPLNHILGRRGTIFLAAVFSLLAPLGSGFTQNWGQLVACRVLLGLGMGLKEVTVPVYSAENAPTNIRGGLVMSWQLWTAFGIFLGTCANLIVANTGDIAWRLQLGSAFIPAVPLLIGIWFCPESPRWLMTKKNHKKAFASLLRLRNSPLQAARDLYRIHAQLEMEKQLIAESGFSKSDNMFVRFVELFTVPRLRRAVQASGIVMIAQQMCGINIISFYSSTIFQLAGASNIEALLASFGFGLINFAFAWPAVWTIDTFGRRGLLLFTFPQMCWTLLAAGFCFWIPTSSNAHIGMIAFFVYLFDAFYSPGEGPVPFTYSAEVFPLSHREVGMAWAVATNNFWASVLSLTFPWMLRAFSPQGAFGFYAGLNLVAFALIFFFMPETMQRSLEELDYVFGVPTRTHAKYQLTQVLPWWIKKYVLMRKDAVCPELYQVTEDYSAAPETVAVPQKHMKADENERHAEYA

SEQ ID NO: 221 - DNA, 1893 bp, Penicillium rubens Wisconsin
  source 1..1893: mol_type = genomic DNA
atgcctaatccaactggaaatgacgcggttgggggaatgaccaagaatgtcaccggggagatcaaaaacccccttgccggaattccccacgatcagcttatggcgaatgtgactgcttatgctaccgaatatgatcttgagagtatcttgccggtcttgaagaaaggtgctctggtagctcagtcaccagcgggaattgaagacctttccgaactcaacgatgacgaccgccgtatcctttacgcggagcacactcggcgctggaaacacccatttgcattgtactacaccatcgttctgaactcaatctcggcagctattcaaggctgggatcagactggttcaaacggtgctaacctgacgtttgacgtccagttcggtatcccgaataactcccctcaatgtcctgacccggagacctgcaagcgtaaccagtggatcgttggttttatcaatgcgtccccatacatcacgatctgtctgtttgttgcttggctctctgaccccctgaatcatatcctcggccgtcgtggtacaattttcctggccgcggtcttcagtctcctagctcccctgggctccggattcactcagaattggggccagctcgttgcctgccgggttcttctcggacttggcatgggtctcaaggaagttactgttcctgtttactcagctgaaaacgcaccaactaacattcgtggcggcctggttatgtcatggcagctttggacggcctttggtatcttcctcggaacgtgcgcgaacctcattgtggccaacactggcgatattgcttggcgactacagctaggctctgcatttattccagctgtgccattgctcattggtatttggttttgccccgagtctccacggtggctgatgaccaagaaaaaccacaagaaagcctttgcatctcttctcagactgcgaaatagccctctacaggccgctcgagatttgtatcggattcacgcccagctggagatggagaagcagttgattgccgagtccggattctccaagagcgacaacatgttcgttcgttttgtggaattgttcacggtgccccgtctgcgtcgtgccgtccaagcctctggcattgtgatgatcgcccagcagatgtgtggaatcaatatcatctcgttctactcctcaactatcttccaacttgctggtgcttccaacatcgaagcacttcttgcatcattcgggttcggccttatcaatttcgcatttgcctggcccgccgtttggacgatcgatacatttggcagacgaggattgctacttttcaccttcccacaaatgtgctggacactcctcgcagctgggttctgtttctggattcccacaagcagcaacgcgcacattggaatgattgcattcttcgtctacctctttgacgctttctattctcctggagagggccctgttcctttcacctactctgccgaggtgtttccgttgtctcaccgtgaagttggaatggcctgggcagttgcaaccaataacttctgggcttctgttctctcgctgacgtttccatggatgctgcgagcatttagtccgcagggtgcctttggattctatgccggcttgaatctcgttgcatttgctttgatattcttctttatgccagaaaccatgcagcgctctcttgaagaactggactatgtgtttggcgttccgacccgaactcatgccaaataccagcttacccaggttctaccatggtggattaaaaagtatgtcctgatgcgcaaggatgcggtctgccccgaactttaccaggtcaccgaggattattccgcagcccccgagaccgttgctgttccacagaagcatatgaaggccgacgagaatgaacgtcacgccgaatatgcttga

SEQ ID NO: 222 - DNA, 1893 bp, synthetic construct
  source 1..1893: mol_type = other DNA
atgcctaaccctacaggcaatgatgccgttggaggaatgactaagaacgttaccggtgagataaagaatccgttagctggaataccgcacgatcagctgatggcaaacgtaactgcgtacgcgacggaatatgatctggagtctattctacccgtcctgaagaagggagctctagtcgctcaatctccagcggggatcgaagaccttagtgaactgaacgatgatgacaggcgtatcttatacgctgaacacacgagacgttggaaacatccctttgcgctttactacacgattgtattaaatagtatatcagctgcgatacagggctgggaccaaaccgggtccaatggggccaatcttacttttgacgtacaatttggtattccgaataactcacctcagtgcccagacccggaaacttgcaaacgtaaccaatggattgtgggttttattaatgctagtccatatatcaccatttgcctgtttgtagcatggctatctgatccgctaaatcacattctgggtagaagaggcaccatcttcttggctgcagttttctctctactagccccattgggctccggattcacacagaattggggtcaactggtggcgtgtcgtgtccttttgggattaggtatgggtcttaaagaagttacggtccccgtatattcagcagaaaacgcccccaccaatattcgtggggggttggtcatgtcttggcagctgtggactgcgttcggaatattcctaggtacctgcgctaatctaatagtcgccaacactggcgatatagcgtggagattacaactaggcagcgcgtttatcccggcagttcctttattgataggtatttggttctgccccgagtcaccccgttggctaatgactaagaagaatcacaagaaggctttcgcctcacttctaaggctaagaaatagccctcttcaggccgcgcgtgatctatatcgtatccacgcgcagctagagatggagaaacagcttatagctgaatctggattttcaaaatcagataatatgttcgtccgtttcgtcgagttgtttacagtccctagattgcgtagagctgtgcaggccagtggaattgtgatgatagcccagcagatgtgtggtatcaacataatttccttctactcttctacaatatttcaacttgcgggtgccagcaatattgaagcactactggccagttttggattcggactaataaattttgccttcgcatggccggcagtctggaccatagacacgttcgggcgtaggggtcttcttctgttcacatttccccaaatgtgctggactcttcttgctgcagggttttgtttctggattccgactagtagcaacgctcacatcggaatgatagcattcttcgtttacttatttgacgcattttactctcccggggaggggcccgtgcccttcacgtatagtgcggaggtgtttccactatcccacagggaggtaggaatggcttgggcagtggccacgaataatttttgggctagcgttttatcattgacctttccctggatgttgagggccttctcaccacagggtgcattcgggttctacgccggactaaacctggttgctttcgccctgatcttcttctttatgcccgagaccatgcaacgttctcttgaggaattagactatgtgttcggagtccctacccgtacgcacgcgaaataccagctaacacaagtattaccttggtggataaagaaatacgttcttatgcgtaaggacgccgtctgtccggaactttatcaagtcacagaagactactccgccgcgcccgagacagttgcggttccccagaaacatatgaaggcagacgaaaatgaaagacacgctgagtacgcatag

SEQ ID NO: 223 - protein, 614 aa, Arabidopsis thaliana
MASTVDSNFFSSVPALHSNLGLLSPDQIELAKILLENGQSHLFQQWPELGVDDKEKLAFFDQIARLNSSYPGGLAAYIKTAKELLADSKVGKNPYDGFSPSVPSGENLTFGTDNFIEMEKRGVVEARNAAFVLVAGGLGERLGYNGIKVALPRETTTGTCFLQHYIESILALQEASNKIDSDGSERDIPFIIMTSDDTHSRTLDLLELNSYFGMKPTQVHLLKQEKVACLDDNDARLALDPHNKYSIQTKPHGHGDVHSLLYSSGLLHKWLEAGLKWVLFFQDTNGLLFNAIPASLGVSATKQYHVNSLAVPRKAKEAIGGISKLTHVDGRSMVINVEYNQLDPLLRASGFPDGDVNCETGFSPFPGNINQLILELGPYKDELQKTGGAIKEFVNPKYKDSTKTAFKSSTRLECMMQDYPKTLPPTARVGFTVMDIWLAYAPVKNNPEDAAKVPKGNPYHSATSGEMAIYRANSLILQKAGVKVEEPVKQVLNGQEVEVWSRITWKPKWGMIFSDIKKKVSGNCEVSQRSTMAIKGRNVFIKDLSLDGALIVDSIDDAEVKLGGLIKNNGWTMESVDYKDTSVPEEIRIRGFRFNKVEQLEKKLTQPGKFSVED

SEQ ID NO: 224 - DNA, 2406 bp, Arabidopsis thaliana
  source 1..2406: mol_type = genomic DNA
ttttgattcgtttccaccagacgaatcttcgtaacgcattttcgttgattcactttccagctttatctcaaactctctctctgtcgtctcgatcgatctcccaaatctcaaattctgaattgattttttttgtttataaacaaagcaaacaaaacacataattttggttcggtcttcttcaattttcaccgacacaaacccgtcttttcctcttctctctcatttctatcaatttttctctacaattccccagtcgtcgaagaagctacagcttgcaatttgctcgtgttctcttccatcattcctttgttaagagcctcttcatggcttctacggttgattccaatttcttctcatccgtcccagctcttcacagtaacctcggacttttgtctcctgatcagatcgaactggcgaagatcttattagaaaatggtcagagtcatctcttccaacaatggcctgaactcggtgttgatgataaagagaagctagcttttttcgatcagattgctcgcttaaattcaagctatcctggaggtttagcagcatacattaagactgcgaaagagcttctcgctgattcgaaagtagggaaaaacccatatgatggtttctctccttctgtcccttcaggagaaaatctcacttttggtactgacaacttcatcgaaatggaaaaaagaggtgtcgtcgaagctaggaatgctgcttttgttctggttgctggtggtcttggcgagcgtcttggttacaatggaatcaaggtggcacttccaagggagacaactacagggacatgcttcttgcagcattacattgaatctatattggctcttcaagaagctagcaacaagatcgattctgatggaagtgagagagacattcctttcattattatgacatctgatgatactcattcacgtacactggacctgttagagttaaactcttattttggaatgaaacctactcaagtacatcttctaaaacaggaaaaagttgcatgtcttgatgataacgatgccagacttgcattagaccctcacaacaaatacagtatccagacaaaacctcatggacatggtgatgtacattcgcttctttattccagtggccttcttcataaatggcttgaggctggtttaaaatgggttttgtttttccaagatacaaatggacttctcttcaatgcaattccagcttctttgggtgtgagtgctaccaaacagtatcatgttaactctctcgctgttcctcgcaaggcgaaggaagctattggaggaattagtaaacttactcacgttgatgggagatctatggtaatcaatgtggagtacaatcaacttgatcctctacttagagcatctggattccctgatggggatgttaattgtgagacgggcttctccccattcccgggaaatattaatcaattgattctggaacttggtccctacaaagacgagttgcaaaaaactggaggggccattaaagagtttgttaatccaaaatacaaggatagcaccaaaacagcatttaaatcttcgactcgtctagagtgtatgatgcaagattatccaaagacattgcctcctacggcacgagttggattcacggtgatggatatatggcttgcttatgcacccgtaaagaataaccctgaagatgctgctaaggtaccaaagggaaacccatatcatagtgctacttctggagaaatggcgatttatcgtgctaacagcctcattcttcaaaaggctggtgtcaaagtggaagagcctgtgaagcaagtcttgaatggccaagaggttgaagtatggtctcgtatcacatggaaacccaaatggggaatgatcttctctgatatcaaaaagaaagtcagcgggaactgcgaggtctctcagagatccacaatggcaatcaagggtcgtaatgtatttatcaaagatctctccttggacggtgctcttattgtagattccattgatgatgcagaggtaaaacttggaggtttgataaagaacaatggttggaccatggagagtgtagattacaaggacacatcggttccagaagaaataagaatcagaggttttagattcaacaaagtggagcaactcgaaaagaagttaactcaacctggcaaattttctgttgaagattgaagttatctttgcacatttatgtgacacttttttaaaaagtgttatttggcaatatgatcaattagcaaattagctatttttttaagagaatgccaaatgatccattaatttgtccttctaatggagagcttaattctttctttcgttttgtttccaacgaaacacttccttttctttgtttttcaacaaaacccgaatatattgtttctttatgtttttctaaatctttattagcctg

SEQ ID NO: 225 - DNA, 1845 bp, synthetic construct
  source 1..1845: mol_type = other DNA
atggcgtccactgtcgattctaattttttttcaagtgtgcctgccttacactcaaatcttgggttactaagccctgaccagattgagttagctaagatattactagagaacgggcagtcacacctgtttcagcagtggcctgaacttggtgttgatgataaagagaaactagccttctttgatcagatagcgagacttaattccagctacccgggggggttggcggcttatataaaaacggccaaagaacttttggcggatagcaaagttggcaagaatccatatgacggctttagcccgtctgtgccctctggagaaaatttgaccttcggtacggataacttcatcgagatggagaaaagaggcgttgtggaagcgcgtaacgcggcttttgttcttgtcgcgggggggttaggagagaggttaggatacaatggcatcaaagttgctttgccgagggagaccaccaccgggacgtgctttctacaacattatattgaaagtatcttggcgttacaagaggccagcaataagatagactctgacggcagtgagagggacatcccctttattattatgacctccgacgatacgcattctagaacgttagatttactggaacttaatagctattttggtatgaagcccactcaagtacacctactaaaacaggagaaggttgcgtgtctagacgataatgacgctagattagcactagatccacacaacaagtacagcatccagacgaagccgcacggacatggcgacgtgcactctctactttactcaagtggattgcttcacaaatggctagaggcgggactaaagtgggtccttttttttcaagacacaaacgggctgttgttcaatgctataccagcttcacttggagtgtccgccactaaacagtatcatgttaacagcctggcggtgccccgtaaagcaaaggaggcgatcggaggaataagtaagctaacgcacgtggatgggagaagcatggttatcaacgtagagtataaccagctggatccactacttagggctagtgggtttcccgacggtgatgtaaattgcgagacaggctttagtccgttcccgggcaacattaaccagttgatcctagagttaggaccctataaggatgaattgcaaaagaccggtggggccattaaagagttcgtaaacccgaaatataaagacagcacgaagactgcgttcaagtcctccactagacttgaatgtatgatgcaggattatccaaagacgttgccaccaacggcacgtgtaggctttacagtcatggacatatggttggcatacgcccccgtcaagaataaccctgaagacgccgcgaaggttccaaaggggaacccctaccattccgcgacgagtggtgagatggcgatttacagggcgaactcactaatacttcagaaagccggcgtgaaagttgaggaaccagtcaagcaagtgctgaatggacaggaggtagaggtatggtcaaggattacgtggaaaccaaagtggggaatgattttctcagacataaaaaagaaggtgagtgggaactgtgaggtatcccagcgtagtaccatggctatcaaagggcgtaacgttttcattaaagaccttagccttgatggggcgttgattgtggactccatagatgacgctgaggttaagctaggtgggctaattaaaaacaatggctggacgatggaaagcgttgattataaagataccagtgtacccgaagagataaggattcgtggattccgtttcaataaggttgagcaacttgagaaaaagttgacacagccgggaaagttttccgtcgaggactag

SEQ ID NO: 226 - protein, 630 aa, Leptospira interrogans
MTNPSNSNLQALREELCTPGLDQGHLFEGWPETVDECNERQIALLTDLYMFSNMYPGGVAQYIRNGHELLARESEEVDFAALEMPPLIFEAPSLHRRTAERTALENAGTAMLCKTVFVLVAGGLGERLGYSSIKVSLPVETATNTTYLAYYLRWAQRVGGKEVPFVIMTSDDTHDRTLQLLRELQLEVPNLHVLKQGQVFCFADSAAHLALDETGKLLRKPHGHGDVHSLIYNATVKRDVVPDSGDGTATAQPLVNDWLAAGYESIVFIQDTNAGATITIPISLALSAEHSLDMNFTCIPRVPKEPIGLLCRTKKNSGDPWLVANVEYNVFAEVSRALNKDGGDEVSDPTGFSPFPGSVNTLVFKLSSYVDRLRESHGIVPEFINPKYSDETRRSFKKPARIESLMQDIALLFSEDDYRVGGTVFERFSYQPVKNSLEEAAGLVAQGNGAYCAATGEAAFYELQRRRLKAIGLPLFYSSQPEVTVAKDAFGVRLFPIIVLDTVCASSGSLDDLARVFPTPEKVHIDQHSTLIVEGRVIIESLELYGALTIRGPTDSMALPHVVRNAVVRNAGWSVHAILSLCAGRDSRLSEVDRIRGFVLKKTAMAVMDCNTKGESEAGAPSGAADPAKL

SEQ ID NO: 227 - DNA, 1893 bp, Leptospira interrogans
  source 1..1893: mol_type = genomic DNA
atgacgaacccgtccaactccaacctgcaggccttgcgcgaggagctctgcacgcctggcctggatcagggtcacctcttcgagggatggccggagactgtggatgagtgcaacgagaggcagatcgccctcctcacagatttgtacatgttttccaacatgtatcccggcggcgttgctcagtacatccgcaacgggcacgagctgctggcgcgtgagagcgaagaggtggactttgcagcgctggagatgccccctctcatcttcgaggcgccgtcgctgcaccggcgcacggctgagaggacggcgctggagaacgccggaaccgcgatgctgtgcaagacggtgttcgtgctggttgctggcggtctgggcgaacgtctgggctactcgagcatcaaggtgagcctgccggtggagacggcgacgaacacaacgtatctcgcctactacctccggtgggcccagcgggtgggggggaaggaggtaccatttgtgataatgacctctgacgacacgcacgaccgcacgctgcagctcctgcgcgagctgcagttggaggtgcccaacttgcatgtgctcaagcaggggcaggtcttctgtttcgccgacagcgccgcgcacctcgccctggacgagacagggaagctgctgcgcaagccacacggtcacggcgacgtgcactccctcatctacaacgcgactgtgaagagagacgtggtgccggactccggcgacggtaccgcgacggcgcagccactcgtgaacgactggctggcggccggctacgagtccattgtcttcatccaggacaccaacgccggcgcgacgatcacaatccccatcagcctcgccttgagtgccgagcactcgctcgacatgaacttcacctgcatccctcgtgtgccgaaggagccgatcgggctgctatgccgaaccaagaagaatagcggcgacccgtggctggtcgcgaacgtggagtacaacgtctttgccgaggtctcgcgcgcgcttaacaaggatggtggcgatgaagtcagtgaccccactggcttctccccgttccctggcagcgtcaacaccctcgtgttcaagctctccagctacgtggaccggctgcgggagtcgcacggtatcgtgccggagttcatcaatcccaagtactcggacgagacgcgccgctccttcaagaagcccgcacgcatcgagtccctgatgcaggacatcgcgctgctcttctccgaggatgactaccgtgtcggcggtaccgtctttgagcgattctcgtaccagccagtgaagaactcgctagaggaggcggcagggcttgtggcgcagggcaacggcgcctactgcgccgccacgggagaggctgccttctacgagctgcagcggcgccgtctcaaggccatcgggctgccgctcttctacagctcgcagccggaggtgacggtggcgaaggacgcctttggcgtgcgtctcttcccgataatcgtgctggatacggtgtgcgcgtcaagcggatccctcgacgaccttgcgcgcgtctttccgacgccggaaaaggtgcacatcgatcagcacagcaccttgattgttgagggccgtgtcatcatcgagagcctggagctatacggtgcactcacgattcgcggcccgacagactcgatggcgctgccgcacgtagtacgaaacgctgtggtgcgcaatgccggctggtcggtacacgcgatcttgtctctctgcgctgggcgcgatagcaggctgtccgaggtggaccgcatccgcgggtttgtgctgaagaagacagccatggcggtgatggactgcaatacgaagggcgagtccgaggccggtgcaccgtctggtgcggctgacccggcaaagttgtag

SEQ ID NO: 228 - DNA, 1893 bp, synthetic construct
  source 1..1893: mol_type = other DNA
atgacaaatccatctaacagcaatcttcaagcattgcgtgaggagctttgtacgcccggattggatcaaggccatttattcgagggctggccagaaactgttgacgaatgcaatgaaagacagattgctcttttaacggacctgtacatgttctccaatatgtatcccggtggggtggcgcaatatataaggaacggacatgagctgctagcgcgtgagtccgaggaggtcgactttgcagcattggaaatgcctcccttgattttcgaagctccgtcattacataggaggacagccgagcgtactgcgttagagaacgctggcactgcaatgctatgcaagacagttttcgtattggtcgcaggggggctgggagagaggttgggctattctagcatcaaagtctcattgccggtcgagaccgcaacgaataccacctatcttgcttactacctgagatgggcccagagggttggggggaaagaggttccatttgtcataatgacatctgacgatacacatgaccgtactctacaactgctacgtgaactacaattagaggtaccgaacctgcatgtgctaaagcagggacaggttttttgctttgcagactctgcagcccaccttgccctggacgagactggaaaacttctacgtaaaccccatggtcacggtgacgtccacagccttatatataatgcgacggttaaaagagatgtggtacccgactcaggcgacggtacggctactgctcagccgttggtaaacgattggcttgctgctgggtatgagtcaatagtatttattcaagacacaaatgcgggagctactatcactattccgataagtctggccctatccgcagaacattctctggatatgaacttcacctgcattccaagagttcccaaagaacccatcgggctactttgcagaaccaagaaaaattctggtgatccctggttagtggctaacgttgagtacaatgtatttgctgaggtgtctagggcgcttaacaaggacggcggggacgaagtttcagacccaacaggcttctccccttttccgggatccgttaatacgttagtattcaaactatcaagctacgtagacaggctaagggaatcacacggaatcgtgcccgaatttatcaatccgaagtattccgatgaaacaaggaggtcttttaaaaagccagccaggatagaatcccttatgcaggatattgcgttactgttctctgaagacgactaccgtgtagggggtacagtctttgaacgtttttcctaccaacccgttaaaaattcacttgaggaagcggcgggcttggtagcgcaagggaatggtgcatattgtgctgccaccggcgaagctgctttctatgagctacaacgtaggcgtttaaaggctattgggcttccacttttttacagtagtcagcctgaagttactgtagcgaaggatgccttcggtgtacgtctatttccgattatcgtgttggacacagtatgcgcttcctccgggagcttagacgacttagcaagggttttccctacgcccgaaaaagtgcatattgaccaacatagtaccttaattgtggaaggtcgtgtaattatcgaatcccttgagttatatggcgctttaactattagaggcccgactgattcaatggccctgccccatgtcgttagaaatgccgtggtgagaaatgcaggctggagtgtccatgccatactgtccttatgcgctggaagggactctaggttaagcgaagttgatagaatcagaggatttgttcttaagaaaactgccatggcagttatggactgcaacacgaagggagaatccgaagcgggcgccccttctggcgcagctgatcccgcaaagttgtaa

SEQ ID NO: 229 - protein, 504 aa, Quillaja saponaria
MPFIRNTPKAKKAAMGSSAKPLHFVLFPHLAPGHLLPLFDTARLLAQRGATVTIFTSPGNASRFESLVSRAVSSGYSIQFVQVQIPFKEAGLPEGCENFDMLQSYDLAFKLYTAIRFMHKPAEILFEALPLKPSCIISDVCVPWTLQIARKHNIPRITFHIFSTFFLLSSYKFGMSQVTQNTFTETEHVAMSEIPDQNDPITEIQTQAQLSQKLMHYSIQMMAADIKSYGAIVNSSEAVEPESIEEYKKLRNGKAWCVGPVWLCNKNELDKAERGNKSSIDEHYCLEWLNLQEENSVVYACLGSVANLSASQLIELGLGLEASKRPFVWVIRGGDNLKELEKWIKEDGFEERIKGKGLLIRGWAPQVLILSHSAIGGFITHCGWGSILEAITCGVPIITWPLFADQFCNQINVAQVLKIGVKVGAEDLVNNGEGEKIEALVKKEDIKRAIEELMDEGEESEERRRRVREMGEMAKKAAEEGGSSHLDLTSLIEDIKQQSTSTQE

SEQ ID NO: 230 - DNA, 1515 bp, Quillaja saponaria
  source 1..1515: mol_type = genomic DNA
atgccattcattcgtaatactccaaaagcaaagaaggcagccatgggttccagtgcaaagcctcttcattttgtattgtttcctcatttggcaccaggccatcttctccccttatttgacactgcaagactgttggcacagcgtggtgctactgttacaatattcacctcaccaggaaacgcatcccgttttgaatcacttgtttctcgtgccgtttcgtcaggctactcaatccaattcgttcaagtccaaattccattcaaagaagcaggactacctgaagggtgtgagaattttgacatgctacagtcttatgacttggcattcaaactttacactgcaattagattcatgcacaagcctgcagaaatattgtttgaagcactaccactcaaaccaagttgcataatctctgacgtctgtgttccatggacacttcaaattgctcgaaaacataatattccaaggattactttccatatcttctcaactttctttctcttgagctcctacaagtttggaatgtcccaggttactcagaacacattcacagaaacagagcatgttgccatgtcagaaattcctgaccaaaatgatccgattacagaaatccagacacaagcacaactgagtcaaaaactgatgcattattctatccaaatgatggctgcagatataaaatcatatggcgcaatcgtaaatagctctgaagcagtggagccagagtctattgaggagtacaaaaagctaaggaatgggaaggcctggtgtgttggccccgtttggctgtgcaataagaatgagttagataaggctgagagaggtaacaaatcctccattgatgagcattattgcttggagtggcttaatttgcaggaagaaaactctgtagtctatgcctgccttggaagtgtagccaacttgagtgcttcacaactaatagaacttggattggggttagaagcatctaaaagaccatttgtttgggttataaggggaggggataatttaaaagaattggaaaagtggattaaggaagatgggtttgaggaaaggattaaagggaaaggtcttttaattagaggttgggcaccacaagtactaattttgtcccactctgccattgggggattcataacacattgtgggtggggttcaatacttgaagcaataacttgtggtgttccgataattacatggcccttatttgctgatcaattttgcaatcagattaatgttgcacaagtgttaaaaattggtgtgaaagttggggcagaggatcttgtaaacaatggagagggagagaagattgaagcattggtgaagaaggaagacattaagagggcaatagaagaactgatggatgaaggtgaagaaagtgaagagagaagaagaagagtgagagaaatgggagagatggcaaagaaagctgcagaagaagggggttcttctcatcttgatctcacttcgcttattgaagatatcaaacaacaatcaactagcacgcaggaatga

SEQ ID NO: 231 - DNA, 1515 bp, synthetic construct
  source 1..1515: mol_type = other DNA
atgccctttattcgtaacacgccaaaagctaaaaaggctgcaatgggctcaagcgcgaagcccttacatttcgtgctgttcccgcatttggcaccgggccacttgctgccgctgtttgacacggcaaggttgttggcacaaagaggcgcgacggtcacaattttcacctcacccggaaacgcgagccgttttgagtcattagtatctcgtgcggtctcctctggctatagcattcagttcgtgcaggtccaaataccgttcaaagaggccggcttacccgagggttgcgaaaattttgacatgctacagagttatgatctggcattcaagctgtataccgcgatcagattcatgcacaagcccgcggagatattattcgaggcgctgcccctgaagccgtcttgcataatctccgacgtgtgtgtcccctggaccttacagatcgcccgtaaacacaatataccaagaataacttttcacatattctcaactttctttttactttcatcctataaattcggtatgtcacaagtgacgcagaacacttttacggaaacagagcatgtggcaatgtccgaaattcctgatcagaatgatcccatcacggaaattcagactcaggctcagctgagccagaaactaatgcactattccatccagatgatggccgcggatatcaaaagttacggagcaatcgtaaatagctctgaggctgtcgaacccgaaagtattgaggaatataagaagcttcgtaatggaaaggcttggtgtgtcgggccggtttggttatgtaataagaacgagctagacaaggcagaacgtgggaataaaagttcaatcgacgagcattattgcctggaatggttaaatctgcaagaagaaaattccgttgtctacgcctgcttaggttctgtggccaatcttagcgcgtcccaactgattgaactagggttaggcctagaagcttccaaaaggcctttcgtatgggtcattcgtggtggcgataatctaaaagaattggagaagtggataaaagaagatggatttgaggagcgtatcaaaggtaaaggcctactaataaggggctgggcaccgcaggtcttaatcttgagccactcagcaatcggcggttttatcactcactgcggctgggggtcaatcctggaggcgataacgtgcggagtaccgatcataacctggccattgttcgctgaccagttttgcaatcaaattaacgtagcacaggtgttgaagatcggcgtgaaggtaggggccgaagatctggtgaataacggcgagggtgaaaagattgaagcattagtgaaaaaggaagacatcaaaagagcgatagaggaactgatggacgaaggcgaggaaagtgaagagcgtagacgtcgtgtgagggaaatgggggagatggcaaagaaggctgccgaagagggcggttcttctcatctagacctaacaagtttaattgaagacattaaacagcaatcaacgagcacgcaagagtga

SEQ ID NO: 232 - protein, 474 aa, Quillaja saponaria
MDAQPNPLHFVIFPHLASGHLIPMVDIARLLAQRGAIITIFITAQNAARFENVLARAVSSGLSIHLVLVPVAFEEAGLPQGYDNFDMLPSFDMQFKLFTAIKLMQNPAEKLFAALKLKPCIISDVCVPWTIEIARKNRIPRISFHAISSFCQLTSYNLGISKVPKNPTSEPSVQLVMPEIPDQVEINTEVQTQAQLDQKLMHYSIQMMVADVNSYGVIVNSFEELEEAYVKDYKKVRKVWCIGPVSLCNKSESDKAERGKKASIDEHQCLEWLNLQQENSVVYACLGSQSNLLPSKLIELALGLEASNRPFIWVIRGGKSTLEELQKWITRNGFEEKIKGRGLLIRGWAPQVLILSHPSIGGFLTHCGWNSILEGISAGVPLITWPVFADQFFNAKHVTQVLKIGVRIWEDEPGWGQEEKIVVRRIEKVIDEGEESEARRKRVRQLGDMANHAVEEGGSSHVNLSLFIQDIMQQ

SEQ ID NO: 233 - DNA, 1428 bp, Quillaja saponaria
  source 1..1428: mol_type = genomic DNA
atggatgcacaaccaaatcctcttcattttgtaatttttcctcacttggcgtcaggacatcttattcctatggttgacattgcaaggctcttagcacaaaggggtgctattatcacaattttcatcacagcacaaaacgcagcccgtttcgaaaatgttcttgctcgtgctgtttcatctgggctttcaatccatttagttcttgttccagttgcatttgaagaagcaggactacctcaagggtatgataattttgacatgctaccctcttttgacatgcaattcaaacttttcactgcaattaaattgatgcaaaatccagcagaaaaattgtttgcagcactaaaacttaaaccagattgcattatctctgatgtatgtgttccttggacaatcgaaattgctcgaaagaatcgtattcctaggatttcttttcatgccatatcaagcttctgtcaattgacatcctacaatttgggcatttccaaggttcccaagaacccaacctcagagccatcagtacaacttgttatgcctgaaattcctgaccaagttgagattaatactgaggtgcagacacaagcacaactggatcaaaagttgatgcactattctattcaaatgatggttgctgatgtaaattcttatggtgtaatcgtaaacagtttcgaagagttggaggaagcatatgttaaggattacaaaaaggtaaggaaggtttggtgtattggcccggtttcgctgtgcaacaaaagtgagtcagataaggctgagagaggtaaaaaagcctcaattgatgagcaccaatgcttggagtggcttaatttgcagcaagaaaactctgtagtctatgcttgccttggaagccaatccaacttgttgccttcaaaactaatagagcttgcgttgggtttagaagcatcaaatagaccttttatttgggttataaggggaggaaaatcaacgttggaagaattgcagaagtggattactagaaatggttttgaggaaaaaatcaaagggagaggtctgttaattagaggttgggcaccacaggtattgatattatcccatccaagtattggtgggttcttaacacattgtggttggaattcaatacttgaagggataagtgctggtgtgccactgattacatggccagtatttgcagaccagtttttcaatgcgaaacatgttactcaagtgttaaaaattggtgtgagaatctgggaagacgaaccgggctggggacaggaagagaagatagtggtgaggcgaatagagaaagtgatagatgaaggcgaagaaagtgaagcgagaagaaaaagggtcagacaacttggtgatatggcaaaccatgctgtagaagaagggggatcttctcatgttaacttgtctttgttcatccaagatataatgcaacaatga

SEQ ID NO: 234 - DNA, 1431 bp, synthetic construct
  source 1..1431: mol_type = other DNA
atggatgcgcagcctaatccactacacttcgtaatttttcctcacttggcatctggacatctaataccgatggttgatatagcaaggttgttggcacagagaggggcgataatcacgatcttcatcaccgcccagaatgcagcacgttttgaaaacgtgctagccagggctgtcagtagcggcctgagcatccacttggtactggtaccggtagcctttgaggaagccggcttaccccaagggtatgacaattttgatatgttgccctcattcgacatgcagtttaagctgttcacggccataaaactgatgcagaatcccgcagaaaagctgttcgcggccttgaagttaaaaccggattgcataatatcagacgtgtgcgttccttggacgatcgaaatcgccaggaaaaaccgtataccgagaatcagttttcatgctattagctccttctgtcaactaactagctacaacctggggatctcaaaggttccaaaaaatccgacttccgaacccagtgtccagctagtgatgccggagatacctgatcaggtagagataaatacggaggtacagacccaagcgcaattggaccagaaactgatgcattattcaattcagatgatggtagcggatgtgaattcttatggggttatagtaaattcttttgaagagctagaggaggcatatgtcaaagattataagaaagttcgtaaggtctggtgcataggtccggtcagcctttgtaataagtctgaatctgacaaggctgagagggggaaaaaagcatccattgacgaacatcaatgcttagagtggttgaaccttcagcaggaaaactccgttgtatatgcctgtttaggtagtcaatcaaatctacttccatccaaactgatcgaattagcccttggattggaggccagcaatagaccgtttatatgggttattcgtggtgggaaatcaacgcttgaggaactgcagaagtggattactcgtaacggttttgaggagaaaattaaaggcagggggctattaattcgtggctgggctccgcaagtgctgattttatctcacccatctattggggggtttctgacgcattgtggatggaattccattctggaagggatttctgccggtgtgccgttgatcacatggccagtttttgcggaccagttttttaatgcgaagcacgtaacacaggtgttgaaaatcggtgttagaatatgggaagacgaaccgggttggggccaagaggagaagattgttgtaagacgtatagaaaaggtcattgacgaaggagaagaaagtgaggctagacgtaaaagggtaaggcagctaggtgacatggcaaatcacgcggtcgaggagggcggtagcagtcatgttaatttgagtcttttcatacaagacatcatgcagcaatagtag

SEQ ID NO: 235 - protein, 2809 aa, synthetic construct
MTPLDAPGAPAPIAMVGMGCRFGGGATDPQKLWKLLEEGGSAWSKIPPSRFNVGGVYHPNGQRVGSMHVRGGHFLDEDPALFDASFFNMSTEVASCMDPQYRLILEVVYEALEAAGIPLEQVSGSKTGVFAGTMYHDYQGSFQRQPEALPRYFITGNAGTMLANRVSHFYDLRGPSVSIDTACSTTLTALHLAIQSLRAGESDMAIVAGANLLLNPDVFTTMSNLGFLSSDGISYSFDSRADGYGRGEGVAAIVLKTLPDAVRDGDPIRLIVRETAINQDGRTPAISTPSGEAQECLIQDCYQKAQLDPKQTSYVEAHGTGTRAGDPLELAVISAAFPGQQIQVGSVKANIGHTEAVSGLASLIKVALAVEKGVIPPNARFLQPSKKLLKDTHIQIPLCSQSWIPTDGVRRASINNFGFGGANAHAIVEQYGPFAETSICPPNGYSGNYDGNLGTDQAHIYVLSAKDENSCMRMVSRLCDYATHARPADDLQLLANIAYTLGSRRSNFRWKAVCTAHSLTGLAQNLAGEGMRPSKSADQVRLGWVFTGQGAQWFAMGRELIEMYPVFKEALLECDGYIKEMGSTWSIIEELSRPETESRVDQAEFSLPLSTALQIALVRLLWSWNIQPVAVTSHSSGEAAAAYAIGALTARSAIGISYIRGALTARDRLASVHKGGMLAVGLSRSEVGIYIRQVPLQSEECLVVGCVNSPSSVTVSGDLSAIAKLEELLHADRIFARRLKVTQAFHSSHMNSMTDAFRAGLTELFGADPSDAANASKDVIYASPRTGARLHDMNRLRDPIHWVECMLHPVEFESAFRRMCLDENDHMPKVDRVIEIGPHGALGGPIKQIMQLPELATCDIPYLSCLSRGKSSLSTLRLLASELIRAGFPVDLNAINFPRGCEAARVQVLSDLPPYPWNHETRYWKEPRISQSARQRKGPVHDLIGLQEPLNLPLARSWHNVLRVSDLPWLRDHVVGSHIVFPGAGFVCMAVMGISTLCSSDHESDDISYILRDVNFAQALILPADGEEGIDLRLTICAPDQSLGSQDWQRFLVHSITADKNDWTEHCTGLVRAEMDQPPSSLSNQQRIDPRPWSRKTAPQELWDSLHRVGIRHGPFFRNITCIESDGRGSWCTFAIADTASAMPHAYESQHIVHPTTLDSAVQAAYTTLPFAGSRIKSAMVPARVGCMKISSRLADLEARDMLRAQAKMHSQSPSALVTDVAVFDEADPVGGPVMELEGLVFQSLGASLGTSDRDSTDPGNTCSSWHWAPDISLVNPGWLEKTLGTGIQEHEISLILELRRCSVHFIQEAMESLSVGDVERLSGHLAKFYAWMQKQLACAQNGELGPESSSWTRDSEQARCSLRSRVVAGSTNGEMICRLGSVLPAILRREVDPLEVMMDGHLLSRYYVDALKWSRSNAQASELVRLCCHKNPRARILEIGGGTGGCTQLVVDSLGPNPPVGRYDFTDVSAGFFEAARKRFAGWQNVMDFRKLDIEDDPEAQGFVCGSYDVVLACQVLHATSNMQRTLTNVRKLLKPGGKLILVETTRDELDLFFTFGLLPGWWLSEEPERQSTPSLSPTMWRSMLHTTGFNGVEVEARDCDSHEFYMISTMMSTAVQATPMSCSVKLPEVLLVYVDSSTPMSWISDLQGEIRGRNCSVTSLQALRQVPPTEGQICVFLGEVEHSMLGSVTNDDFTLLTSMLQLAGGTLWVTQGATMKSDDPLKALHLGLLRTMRNESHGKRFVSLDLDPSRNPWTGDSRDAIVSVLDLISMSDEKEFDYAERDGVIHVPRAFSDSINGGEEDGYALEPFQDSQHLLRLDIQTPGLLDSLHFTKRNVDTYEPDKLPDDWVEIEPRAFGLNFRDIMVAMGQLESNVMGFECAGVVTSLSETARTIAPGLAVGDRVCALMNGHWASRVTTSRTNVVRIPETLSFPHAASIPLAFTTAYISLYTVARILPGETVLIHAGAGGVGQAAIILAQLTGAEVFTTAGSETKRNLLIDKFHLDPDHVFSSRDSSFVDGIKTRTRGKGVDVVLNSLAGPLLQKSFDCLARFGRFVEIGKKDLEQNSRLDMSTFVRNVSFSSVDILYWQQAKPAEIFQAMSEVILLWERTAIGLIHPISEYPMSALEKAFRTMQSGQHVGKIVVTVAPDDAVLVRQERMPLFLKPNVSYLVAGGLGGIGRRICEWLVDRGARYLIILSRTARVDPVVTSLQERGCTVSVQACDVADESQLEAALQQCRAEEMPPIRGVIQGAMVLKDALVSQMTADGFHAALRPKVQGSWNLHRIASDVDFFVMLSSLVGVMGGAGQANYAAAGAFQDALAEHRMAHNQPAVTIDLGMVQSIGYVAETDSAVAERLQRIGYQPLHEEEVLDVLEQAISPVCSPAAPTRPAVIVTGINTRPGPHWAHADWMQEARFAGIKYRDPLRDNHGALSLTPAEDDNLHARLNRAISQQESIAVIMEAMSCKLISMFGLTDSEMSATQTLAGIGVDSLVAIELRNWITAKFNVDISVFELMEGRTIAKVAEVVLQRQLDSGTPAREASSALRDGYRQAGVSGRVRSYLDLLAGLSDFREHFDGSDGFSLDLVDMADGPGEVTVICCAGTAAISGPHEFTRLAGALRGIAPVRAVPQPGYEEGEPLPSSMAAVAAVQADAVIRTQGDKPFVVAGHSAGALMAYALATELLDRGHPPRGVVLIDVYPPGHQDAMNAWLEELTATLFDRETVRMDDTRLTALGAYDRLTGQWRPRETGLPTLLVSAGEPMGPWPDDSWKPTWPFEHDTVAVPGDHFTMVQEHADAIARHIDAWLGGGNS

SEQ ID NO: 236 - DNA, 8430 bp, synthetic construct
  source 1..8430: mol_type = other DNA
atgacaccattagatgcgcccggtgcgcctgctcccatagctatggttggcatgggctgcagatttggcggaggcgcaacagatccccagaaactgtggaaattgctggaggaaggagggagcgcctggtctaagattcctccttcacgattcaatgtcggcggggtctaccaccccaatggccagcgggtaggatcgatgcacgttcgcggtggacactttctcgacgaagacccggctcttttcgatgcctcatttttcaatatgagtactgaagttgccagttgtatggacccccagtaccgactcatacttgaagtcgtttatgaggcgctcgaagctgcgggaattcctctcgaacaggtctccggctccaagactggggtttttgcaggaaccatgtatcacgactaccaaggctccttccagcgccaaccagaagcccttccacggtatttcataacaggaaatgctggcaccatgctcgcgaatcgcgtctcccacttttatgaccttcgtgggcccagtgtctcgatcgacactgcctgttccacaaccttaacagccttgcatcttgccattcagagcttgcgagctggagaatctgatatggcgattgtcgctggcgcgaacctgctacttaatcctgacgtctttactaccatgtccaaccttggcttcctttcgtccgatgggatttcctactcatttgactcgagagcggatggctatggtcgcggagaaggagtggctgcgatcgtcttgaagactctgcccgatgcggtgcgagacggagacccgatccgcctcatagtgcgcgaaacggcaatcaaccaagacggccggaccccagccatcagcacgccgagcggcgaggcccaggagtgcctgatccaagattgctatcagaaggcccagttggacccaaaacagacttcgtacgttgaggcccatgggacgggaaccagagcaggagatccgctggagcttgcagtcatctcggccgcgtttccgggacagcagatacaggtgggctccgtgaaagccaatatcgggcatacagaggctgtcagtggtctggcgagtttgataaaggtggctctggctgttgaaaagggggttatcccgcctaatgcaaggttcctccagccgagcaagaagttgctcaaggacactcatatccagattccactgtgtagccaatcatggataccaaccgatggtgtccgtcgcgcatcaataaacaacttcggtttcggaggcgcaaatgctcatgcaatcgtggagcaatatggcccgtttgcagaaacatcgatctgcccacctaatggttattctggcaactatgatggcaatttaggaacggatcaagcgcatatatatgtgctgagtgccaaggatgagaacagttgcatgagaatggtttcaaggctgtgcgactatgctacccacgccagaccagccgacgatttgcaattgctcgcgaatatagcatacacgcttggttctcgtcgctcgaacttccgatggaaggcagtatgtacggcacacagcctcacgggtcttgcccagaatttggcgggagaaggcatgcggccaagcaagtcagccgaccaagtaagactgggatgggtgttcacaggccagggagcgcaatggtttgcaatgggtcgtgagttgattgagatgtatcctgtctttaaagaggccctgctggaatgcgatggatatatcaaggaaatggggtcaacctggtccattatagaggaactcagtcgccctgaaacggaaagtcgcgttgatcaggcagaattcagtctgccattgtctacggctcttcaaattgcgcttgttcgtctgctctggtcgtggaacatccaaccagtagccgtcactagtcactccagcggagaggcagctgcagcgtacgctatcggggcactaacagcccgctcggccattggaataagctatatacgcggtgcattgacagcaagagaccgcctggcgtcggtacataaggggggcatgttggctgtcggattgagccgcagtgaagtgggtatatacatcagacaggttccattacagagtgaagaatgcttggtggtggggtgtgtcaacagcccgtcgagtgtgacggtctcgggagatttgtccgccattgccaagttggaggaactgctccatgctgatcgtatatttgcgagacggctgaaagtcacccaagcctttcactccagccacatgaactcgatgacagatgctttccgagccggtcttacagaactcttcggagcagaccccagtgatgcagcaaacgccagtaaagatgtgatctacgcttctcccagaaccggggcccgcctgcacgacatgaatcgtcttcgggatcctatacactgggtcgaatgcatgcttcacccggttgagttcgaatcagcattccgtcgaatgtgcctggacgaaaacgaccacatgccaaaggtcgatagggtcattgagattggacctcacggagcgcttggaggcccgatcaagcagatcatgcagcttccagagcttgccacgtgtgacatcccttatctgtcctgtctttctcgtgggaagagctctctgagcacccttcgccttctcgcatcagaacttatccgggccggatttcctgttgacttgaatgcgatcaactttccccgcggatgtgaagcagctcgggtccaagtgttgtctgatctaccgccctacccttggaaccacgagaccagatactggaaagagccgcgcatcagccaatctgcccggcagcggaagggcccagtccacgatctgatcggattgcaggagccgttgaacctgccgttggcgcggtcatggcacaatgtgcttcgtgtgtcagatttgccatggctacgcgaccacgtcgtcggctcgcatattgttttccctggggctgggttcgtgtgtatggcagtgatgggaatcagcacgctctgctcgtccgaccatgaatctgacgacatcagttacatcctacgcgacgtgaactttgcgcaggccctgattctacctgcggacggggaagaaggaatagatctgcgcctcacgatttgtgctcccgatcagagtctgggttcacaggactggcaaagattcttagttcattcgatcactgctgacaagaatgactggacggaacactgtacgggacttgttcgagcagagatggaccagcctccctccagtttgtcgaaccaacaacggatagacccacggccatggagccgtaaaacggcgccgcaggagctgtgggactcactacatcgggtgggaattcgtcatgggcccttttttcgaaacattacgtgcatcgaaagcgacgggcgagggtcatggtgtacatttgccatcgcggacacggcctccgcaatgccacacgcctacgaatcccagcacattgttcacccaaccacactagactctgcagttcaggcagcctataccactcttccattcgctgggagccggatcaaatctgcgatggtccccgctcgcgtcggctgcatgaagatttcctcccgacttgcagatttggaggccagggacatgctgcgcgcacaagcgaagatgcacagccaaagtccttccgcattggtaaccgatgtagcagtttttgatgaggcagatccggttggagggcctgttatggagctcgaagggctggtctttcagtctctgggggcaagtctgggcacttctgaccgggactccaccgaccccgggaatacttgcagctcctggcattgggctccagacatcagcttagttaaccccggctggcttgaaaaaaccctgggcacaggtattcaggagcacgagatcagcctcatattggagcttcgacggtgttcggtgcacttcattcaagaggccatggaaagtttgagcgtaggcgatgtcgagaggctgagtggtcatctggccaaattctatgcgtggatgcagaaacaactggcgtgtgcccaaaatggcgagctggggccagagagctccagctggactcgggatagcgagcaggcaagatgcagcctccgctctagagtggttgctggtagcaccaacggcgaaatgatctgtcgcctgggctccgtgctccccgctatcctacgtcgggaagttgatccgttggaggtgatgatggatggccacctgttgtcccgctactatgtcgatgccctcaagtggagtcggtccaacgcgcaagccagcgagctcgtgcgcctctgctgccacaaaaacccgcgcgctcgcatactggaaatcggcggaggcaccgggggttgcacccagctggtcgtggactccttgggcccaaatccgccggtaggccgctatgactttactgacgtctcggccgggttttttgaagcagcccgcaagcggttcgcgggatggcagaatgtgatggattttcggaagttggacatcgaggacgatccagaagcgcaggggtttgtgtgcggatcctacgacgtggtgttggcttgtcaggtcctgcatgccacttctaacatgcagcgcacattgactaatgtgcgcaagctgttgaagccaggaggcaaactcattcttgtcgaaaccaccagagacgagcttgacttgtttttcactttcgggcttctgcccggctggtggctcagcgaagaaccagaaagacagtcgactccgtcactaagccctacgatgtggcgcagcatgctgcacactactggattcaatggtgtggaagttgaggctcgtgactgcgatagccacgagttctatatgattagcaccatgatgtccacggccgtacaggcgactccgatgtcatgctcggtcaaattgcctgaagtgctcttggtctatgttgactcatctacgcccatgtcttggatatcagatttgcagggagagattcgcggcaggaattgttccgtcacttcgctacaggcacttcgtcaagttcctcctaccgagggccaaatatgcgtattccttggagaggtggaacactccatgcttggttcagtcaccaacgacgacttcacacttttgacctcaatgctacagctggctgggggaactttatgggtcacccaaggagcgacaatgaagtctgatgatcccctgaaggctctacacctcggattactacgtaccatgcgtaatgaaagccatggcaagcgatttgtctcacttgacctcgacccttcgcgtaatccatggacaggcgattcgcgcgatgccattgtcagtgttctggatttaattagcatgtcagatgaaaaggagtttgactatgcagagcgggatggagttatccatgttcctcgggcatttagtgactccatcaatggaggcgaggaagacgggtatgccttggagccattccaggacagccagcatctcctgcgactagatatacagactcctgggctcctcgattccctgcacttcacaaagcgcaatgtggacacatatgaaccagataaattaccggacgactgggtagagattgaaccgagggcgtttggtcttaacttccgtgacatcatggtcgcgatgggtcaattggaatcaaacgtcatgggcttcgaatgcgccggcgtggttacaagtctcagcgagacagcaagaacaattgcacccgggcttgcggtcggagatcgggtttgcgccctcatgaacggacactgggcgtcgagggtgaccacaagccggaccaacgtggtgcgcattccagagactcttagtttcccgcatgctgcctccatccctctggccttcacaacagcttacatttcactttacaccgttgcccgcattctgccaggtgaaacggtgttgatccatgccggggcaggaggcgtaggccaggcggccattattcttgctcaattaaccggtgctgaagtctttacaactgctggcagtgagaccaagcgtaaccttttgatcgataaattccacctcgaccctgatcatgtcttctcgagcagggactccagcttcgtcgacggtatcaagacccgcacccgtggcaagggggtggacgtggttttgaactcgctagctgggcctctccttcagaagagctttgactgtctggctaggtttggtcggtttgtagaaatcggcaagaaggatcttgagcagaatagccgactcgacatgtcgacgttcgtccgcaatgtctccttctcctccgttgatattctctactggcagcaagcgaagcccgctgaaatcttccaggcgatgtccgaggtcatcttgctgtgggagcgaacggcaatcggcctgattcatccaatatcagagtatcctatgtcggccctggagaaggcctttcgcactatgcagagcggccagcacgttgggaagattgttgtgacagtagcccccgatgacgcggtcctcgttcgtcaggaacgaatgccactatttctgaagcctaacgtgtcgtatcttgttgctgggggcctgggtggtatcggacggcggatctgcgagtggctggtcgatcgcggggcgcgatatctcatcattctgtctcgaactgctcgcgtggacccggtcgtgacgagtctccaagagcggggctgcaccgtttctgtacaggcgtgtgatgtggccgatgaaagccagcttgaagcggctctccaacagtgtcgggcggaggaaatgcctccgattcggggcgtcatccaaggggcaatggttctcaaggacgccctcgtctcgcaaatgacggcggacgggttccatgccgccctgcggcccaaggttcagggaagttggaatctgcaccgaattgcatcggacgtggatttcttcgtgatgctctcatccttggtgggtgtcatgggaggcgcaggacaagccaactacgcggctgccggagcgtttcaggacgcgctcgcagagcaccgcatggctcacaaccagccagcggtcaccatcgacctcggaatggtccagtcaattgggtatgtagcagagacagattctgctgtggcggaacgactccaacggatcggctatcaacccttgcacgaagaggaggttctggacgtcctcgagcaagctatatctcctgtgtgttcccctgccgcacccacacggcctgctgtcatcgtcaccggcatcaacacccgcccaggccctcactgggcacacgccgactggatgcaagaggctcgctttgcggggatcaagtatcgtgatccgttgagggacaatcatggagctttgtcgctgaccccggcggaagatgacaatcttcacgccaggctgaaccgtgcaatcagccaacaggagtcaatcgccgtgatcatggaggcgatgagctgcaagctcatctcaatgttcggcctgacggatagcgaaatgtccgccactcagacattggcggggatcggcgtggactccctggtcgccattgagctccggaactggatcacagctaagttcaatgttgatatctcagttttcgagttgatggagggccgaacgatcgccaaagtcgcggaagtggtgctgcagagacagctggactccggtacgcctgcacgtgaagctagctctgcactgcgtgacggctaccgccaagcgggtgtgagcggccgtgttcgtagctacctggatctgttggcaggtctgtcggatttccgcgaacattttgatggtagcgatggttttagcctggatctggttgatatggcagatggtccgggtgaggtgaccgttatttgctgcgcgggtacggctgcgatctctggtccgcatgagttcacccgtctggcaggtgccctgcgcggcattgcacctgttcgtgcggtgccgcagccgggttacgaagaaggcgaaccgctgccttctagcatggcggctgttgcagctgtgcaggctgatgctgtcattcgtacgcaaggcgataagccgttcgtggttgcgggtcactcggcaggcgcgctgatggcgtacgcgctggcgaccgaactgctggatcgtggtcacccgcctcgcggtgttgtcctgattgatgtgtacccgcctggtcatcaggacgcgatgaacgcgtggctggaggaactgacggcaacgttgttcgaccgtgaaactgttcgtatggacgacacccgtctgaccgcgttgggtgcttacgaccgcctgacgggtcaatggcgtcctcgcgaaaccggtttgccgaccctgttggttagcgcgggtgaaccaatgggcccgtggccggacgatagctggaaaccgacctggcctttcgagcacgacaccgttgcggtcccgggtgatcatttcacgatggttcaagaacatgctgatgcgattgcccgtcacattgacgcctggctgggtggcggcaacagctga

SEQ ID NO: 237 - protein, 344 aa, Aspergillus nidulans
MVQDTSSASTSPILTRWYIDTRPLTASTAALPLLETLQPADQISVQKYYHLKDKHMSLASNLLKYLFVHRNCRIPWSSIVISRTPDPHRRPCYIPPSGSQEDSFKDGYTGINVEFNVSHQASMVAIAGTAFTPNSGGDSKLKPEVGIDITCVNERQGRNGEERSLESLRQYIDIFSEVFSTAEMANIRRLDGVSSSSLSADRLVDYGYRLFYTYWALKEAYIKMTGEALLAPWLRELEFSNVVAPAAVAESGDSAGDFGEPYTGVRTTLYKNLVEDVRIEVAALGGDYLFATAARGGGIGASSRPGGGPDGSGIRSQDPWRPFKKLDIERDIQPCATGVCNCLS

SEQ ID NO: 238 - DNA, 1035 bp, Aspergillus nidulans
  source 1..1035: mol_type = genomic DNA
atggtgcaagacacatcaagcgcaagcacttcgccaattttaacaagatggtacatcgacacccgccctctaaccgcctcaacagcagcccttcctctccttgaaaccctccagcccgctgatcaaatctccgtccaaaaatactaccatctgaaggataaacacatgtctctcgcctctaatctgctcaaatacctcttcgtccaccgaaactgtcgcatcccctggtcttcaatcgtgatctctcgaaccccagatccgcacagacgaccatgctatattccaccctcaggctcacaggaagacagcttcaaagacggatataccggcatcaacgttgagttcaacgtcagccaccaagcctcaatggtcgcgatcgcgggaacagcttttactcccaatagtggtggggacagcaaactcaaacccgaagtcggaattgatattacgtgcgtaaacgagcggcagggacggaacggggaagagcggagcctggaatcgctacgtcaatatattgatatattctcggaagtgttttccactgcagagatggccaatataaggaggttagatggagtctcatcatcctcactgtctgctgatcgtcttgtggactacgggtacagactcttctacacttactgggcgctcaaagaggcgtatataaaaatgactggggaggccctcttagcaccgtggttacgggaactggaattcagtaatgtcgtcgccccggccgctgttgcggagagtggggattcggctggggatttcggggagccgtatacgggtgtcaggacgactttatataaaaatctcgttgaggatgtgaggattgaagttgctgctctgggcggtgattacctatttgcaacggctgcgaggggtggtgggattggagctagttctagaccaggaggtggtccagacggaagtggcatccgaagccaggatccctggaggcctttcaagaagttagatatagagcgagatatccagccctgtgcgactggggtgtgtaattgcctatcctaa

SEQ ID NO: 239 - DNA, 1035 bp, synthetic construct; identical to SEQ ID NO: 238
  source 1..1035: mol_type = other DNA

SEQ ID NO: 240 - protein, 412 aa, Hordeum vulgare
MPTTPRSRSQARTTRSWILSGMDFGDTRRKPNFAGKIAVAAALTVMCILVLKQSPGFGGTSVFSRHETGVTHVLVTGGAGYIGSHATLRLLTDNYRVTIVDNLSRGNMGAVRALQRLFPEPGRLQFIYTDLGDAKAVNKMFSENAFDAVMHFAAVAYVGESTQEPLRYYHNITSNTLTVLEAMAAHNVKTLIYSSTCATYGEPDTMPIVETTPQIPINPYGKAKKMSEDIILDFSKKSNMAVMILRYFNVIGSDPEGRLGEAPRPELREHGRISGACFDAASGIIPGLKVRGTDYPTADGTCVRDYIDVTDLVDAHVKALGKAEPNKVGIYNVGTGKGRSVKEFVEACKKATGVTIKVDYLDRRPGDYAEVYSNPPKIRDELNWTAQHTDLRESLATAWKWQKAHPGGYGSA

SEQ ID NO: 241 - DNA, 1239 bp, Hordeum vulgare
  source 1..1239: mol_type = genomic DNA
atgccaacaaccccccggagcagaagccaagctaggaccaccaggtcttggatcttgtcaggcatggacttcggcgacacgagacgcaagcccaatttcgccggaaagatcgcggtggctgccgccctcactgtcatgtgcatacttgtgttgaaacagtctcctggttttggcggtactagcgtgttctctcgccatgaaactggggtcactcatgtgctagtgactggaggtgctggatacattggctcacatgctactcttcgtcttctcacggacaactaccgagttaccattgtggataacctttctagagggaacatgggagctgtcagagctcttcagaggctgttcccagagcctgggaggcttcagtttatatacactgatttaggagacgcaaaagctgtgaacaaaatgttttcggagaacgcgtttgatgctgttatgcactttgccgcggttgcttatgttggtgagagcacacaggagccactaaggtactaccacaacataacatcaaatacgttgacagtgcttgaggcaatggcagcacataatgtaaaaactctgatttactcgagtacttgtgcaacatatggtgaacctgacacaatgcctattgtcgaaacaactcctcagatacctatcaatccatatggcaaggcaaaaaagatgtccgaggacatcattcttgatttctcaaagaaatcgaacatggctgtgatgatcttaagatacttcaatgtgattgggtcggaccctgagggacgccttggggaagctccaaggcctgaactgcgtgagcatggaaggatttctggtgcgtgttttgacgcagcatcaggaatcattccagggctaaaggttcgaggaacggactaccctactgctgatggaacatgcgtaagggactacatagatgtcactgatcttgttgatgctcatgtcaaagctcttggtaaagctgagcctaacaaagttgggatctacaacgtcggcacggggaaaggtaggtcagtgaaggagtttgtagaagcgtgcaagaaggcgaccggagtgaccatcaaggtcgactacctcgaccggagacccggggactacgccgaagtgtacagcaacccgcccaagatccgcgacgagctgaactggacagcccaacacacggacctccgcgagagcctcgcgacggcctggaaatggcagaaggcgcacccgggcggatacgggtcggcctga

SEQ ID NO: 242 - protein, 421 aa, Hordeum vulgare
MLPAGRSRPQPRPAPRSWSFSDMDFSDPKRKSRYLSKIIMVALLTAMCVVMLTQPPCHRKAPSVFSIHELGVTHVLVTGGAGYIGSHAALRLLNDSFRVTIVDNLSRGNIGAVKVLQNLFPDPGRLQFIHADLGDAKAVNRIFAENAFDAVMHFAAVAYVGESTLEPLRYYHNITANTLVVLEAMAMHNVKTLIYSSTCATYGEPEKMPITEETPQFPINPYGKAKKMAEDIILDFSKSRKSDMSVMILRYFNVIGSDPEGRLGEAPPPELREHGRISGACFDAALGIIPGLKVKGTDYETPDGTCVRDYIDVTDLVDAHVKALNKAERGKVGIYNVGTGRGRSVKEFVEACKKATGVDIKVHYFPRRPGDYAEVYSNPARINRELNWTAQHTELQESLRVAWTWQKKHRSGYADSGRYIL

SEQ ID NO: 243 - DNA, 1266 bp, Hordeum vulgare
  source 1..1266: mol_type = genomic DNA
atgctgccggccggcaggagcaggccgcagcccaggcctgcgccgagatcttggtccttctcagacatggacttctcggacccgaagcgcaagtccaggtacctcagcaagatcatcatggtggcgctcctcacggccatgtgcgtcgtcatgctcacccagccaccctgccacaggaaggctcccagtgtgttctctatccatgaactcggagtaactcatgtgttggtgacgggtggcgccggctacataggatcgcatgcggctctacgactgctcaatgactctttcagagtcaccattgtggataatctctcaagaggaaatattggggcggtcaaggttcttcagaacttgttccctgatcctgggcgattgcagttcatacatgctgatctaggcgatgcaaaagctgttaacagaatattcgcagaaaatgcatttgatgctgtcatgcactttgctgccgttgcttatgtgggcgagagcacgcttgagcctctgaggtactatcataacatcactgcaaatactctggtcgtgctagaagccatggcgatgcacaatgttaaaaccctgatctactctagcacatgtgccacctacggggaacccgagaagatgcctatcactgaagaaactccccagtttcccataaacccgtatggcaaggccaagaaaatggcagaggatatcattttggacttctccaagtccaggaaatcagacatgtcagttatgatcctgagatacttcaacgtcatcggttctgacccagaaggaagactgggtgaggctccaccacctgaattgcgcgagcatggccgtatatctggcgcatgcttcgacgcagcactaggaataatcccaggtttgaaggtcaaaggtaccgactacgaaactcccgacgggacttgcgtgagagattacatcgatgtcaccgacctggtagatgcccacgtgaaagcgctcaacaaggcggagagaggcaaagttggcatatacaatgttggcaccggaagaggtaggtcagtgaaggagtttgtggaagcttgcaagaaggcgaccggagtcgacatcaaggtccactacttcccgcgccgccccggtgactatgcggaagtgtacagcaaccccgcaaggatcaaccgcgaactaaactggacagcccagcacaccgaactccaggagagcctcagggtcgcgtggacatggcagaagaagcaccggagcggctatgcagactcgggtcgatatattctttga